(19) United States
(12) Patent Application Publication
(10) Pub. No.: US 2024/0240182 A1
(43) Pub. Date: Jul. 18, 2024

(54) HUMAN CHROMOSOME 9 OPEN READING FRAME 72 (C9ORF72) iRNA AGENT COMPOSITIONS AND METHODS OF USE THEREOF

(71) Applicants: Alnylam Pharmaceuticals, Inc., Cambridge, MA (US); Regeneron Pharmaceuticals, Inc., Tarrytown, NY (US)

(72) Inventors: Lan Thi Hoang Dang, Arlington, MA (US); James D. McIninch, Burlington, MA (US); Tuyen M. Nguyen, Milton, MA (US); Aarti Sharma-Kanning, Tarrytown, NY (US); David Frendewey, Tarrytown, NY (US); Brittany Savage, Tarrytown, NY (US)

(21) Appl. No.: 18/525,924

(22) Filed: Dec. 1, 2023

Related U.S. Application Data
(63) Continuation of application No. PCT/US2022/031519, filed on May 31, 2022.
(60) Provisional application No. 63/196,791, filed on Jun. 4, 2021.

(51) Int. Cl. C12N 15/113

(52) U.S. Cl. CPC C12N 15/113; C12N 2310/14; C12N 2310/315; C12N 2310/321; C12N 2310/322; C12N 2310/3515

(57) ABSTRACT

The disclosure relates to double stranded ribonucleic acid (dsRNAi) agents and compositions targeting a human chromosome 9 open reading frame 72 (C9orf72) gene, as well as methods of inhibiting expression of a C9orf72 gene and methods of treating subjects having a C9orf72-associated disease or disorder, e.g., C9orf72 amyotrophic lateral sclerosis, frontotemporal dementia or Huntington-Like Syndrome Due To C9orf72 Expansions, using such dsRNAi agents and compositions.

### RELATED APPLICATIONS

This application is a 35 § U.S.C. 111(a) continuation application which claims the benefit of priority to PCT/US2022/031519, filed on May 31, 2022, which, in turn, claims the benefit of priority to U.S. Provisional Application No. 63/196,791, filed on Jun. 4, 2021. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Mar. 8, 2024, is named 121301_14502_SL.xml and is 8.544,874 bytes in size.

### BACKGROUND OF THE INVENTION

Human chromosome 9 open reading frame 72 (C9orf72) is a protein encoded by the c9orf72 gene. C9orf72 is found in many regions of the brain, such as the cerebral cortex, in the cytoplasm of astrocytes and neurons as well as in presynaptic terminals.

Differential use of transcription alternative start and termination sites generates three sense RNA transcripts from C9orf72 DNA. These encode two protein isoforms consisting of a long isoform (isoform A) of approximately 54 kDa derived from variants 2 (NM_018325.4) and 3 (NM_001256054.2), and a short isoform (isoform B) of approximately 24 kDa derived from variant 1 (NM_145005.6) (see, e.g., FIG. 1 of Barker, et al. (2017) Frontiers Cell Neurosci 11:1-15). In addition to the sense RNA transcripts from C9orf72 DNA, there are repeat-containing antisense RNA transcripts, which have been shown to be elevated in the brains of C9orf72 expansion-positive patients. There are also non-repeat-containing sense and antisense RNA transcripts depending on the location of the transcriptional start site.

The two alternatively used first exons of the C9orf72 gene are exons 1a and 1b (see, e.g., FIG. 1 of Barker, et al., supra). A large GGGGCC (G+C2) hexanucleotide repeat expansion (SEQ ID NO: 100) (from about 2-22 copies to 700-1600 copies) in the first intron of the C9orf72 gene between exons 1a and 1b has been shown to 1) interfere with the transcription of the non-repeat containing C9orf72 mRNA, thus decreasing the mRNA and protein levels of C9orf72, 2) generate toxic dipeptide repeat proteins through RAN-initiated translation as well as 3) generate nuclear and cytoplasmic RNA foci, both of which may be pathogenic and result in several neurodegenerative diseases with distinct clinical features but common pathological features and genetic causes (Ling, et al. (2013) Neuron 79:416-438). Furthermore, the repeat-containing antisense RNA transcripts have been shown to accumulate in nuclear and cytoplasmic RNA foci, as well as contribute to the expression of antisense toxic dipeptide repeat proteins through RAN-initiated translation. In particular, the presence of a hexanucleotide repeat expansion in the C9orf72 gene is the most common genetic cause of familial and sporadic Amyotrophic lateral sclerosis (ALS), a devastating degenerative disease of motor neurons in the brain and spinal cord. Indeed, C9orf72 mutation hexanucleotide repeat expansions are present in approximately 40% of familial ALS and 8-10% of sporadic ALS subjects. Hexanucleotide repeat expansion in the C9orf72 gene is also the most common familial cause of Frontotemporal Dementia (FTD), the second most common form of presenile dementia after Alzheimer's disease which is characterized by behavioral and language deficits and manifests pathologically by neuronal atrophy in the frontal and anterior temporal lobes in the brain. Huntington-Like Syndrome Due To C9orf72 Expansions, characterized by movement disorders, including dystonia, chorea, myoclonus, tremor and rigidity, cognitive and memory impairment, carly psychiatric disturbances and behavioral problems, is also associated with hexanucleotide repeat expansion in the C9orf72 gene.

Although the functions of the C9orf72 protein are still being investigated, C9orf72 has been shown to interact with and activate Rab proteins that are involved in regulating the cytoskeleton, autophagy and endocytic transport. In addition, numerous cellular pathways have been demonstrated to be misregulated in neurodegenerative diseases associated with C9orf72 hexanucleotide repeat expansion. For example, altered RNA processing has consistently appeared at the forefront of research into C9orf72 disease. This includes bidirectional transcription of the repeat sequence, accumulation of repeat RNA into nuclear foci sequestering specific RNA-binding proteins (RBPs) and translation of RNA repeats into dipeptide repeat proteins (DPRs) by repeat-associated non-AUG (RAN)-initiated translation. Additionally, disruptions in release of the C9orf72 RNA from RNA polymerase II, translation in the cytoplasm and degradation have been shown to be disrupted by C9orf72 hexanucleotide repeat expansion. Furthermore, several alterations have been identified in the processing of the C9orf72 RNA itself, in terms of its transcription, splicing and localization (see, e.g., Barker, et al., supra).

Irrespective of the mechanism, several groups have identified the presence of sense and antisense C9orf72-containing foci as well as the presence of aberrant dipeptide-repeat (DPR) proteins (poly(GA), poly(GR), poly(GP), poly(PA), and poly(PR)) produced from all reading frames of either sense or antisense repeat-containing C9orf72 RNAs through repeat-associated non-AUG-dependent (RAN) translation in several cell types in the nervous systems of subjects having a C9orf72-associated disease (Lagier-Tourenne, et al. (2013) Proc Natl Acad Sci USA doi/10.1073/pnas. 1318835110; Jiang, et al. (2016) Neuron 90:535-550). Furthermore, in mice with one allele of C9orf72 inactivated no disease was observed but, in mice with both C9orf72 alleles inactivated, splenomegaly, enlarged lymph nodes, and mild social interaction deficits, but no motor dysfunction was observed. In addition, in mice expressing human C9orf72 RNAs with up to 450 GGGGCC repeats (SEQ ID NO: 101) it was shown that hexanucleotide expansions caused age-, repeat-length-, and expression- level-dependent accumulation of sense and antisense RNA-containing foci and dipeptide-repeat proteins synthesized by AUG-independent translation, accompanied by loss of hippocampal neurons, increased anxiety, and impaired cognitive function (Jiang, et al. (2016) Neuron 90:535-550).

There is currently no cure for subjects having a C9orf72-associated disease, e.g., C9orf72 amyotrophic lateral sclerosis, C9orf72 frontotemporal dementia or Huntington's disease, e.g., Huntington-Like Syndrome Due To C9orf72 Expansions, parkinsonism, olivopontocerebellar degeneration, corticobasal syndrome, or Alzheimer's disease, and treatments are only aimed at alleviating the symptoms and improving the patient's quality of life as the disease progresses.

Accordingly, there is a need in the art for agents that can selectively and efficiently inhibit the expression of the C9orf72 gene, e.g., hexanucleotide-repeat-containing C9orf72 RNAs, for, e.g., the treatment of subjects having a C9orf72-associated disorder.

### BRIEF SUMMARY OF THE INVENTION

The present disclosure provides iRNA compositions, which effect the RNA-induced silencing complex (RISC)-mediated cleavage of RNA transcripts of a C9orf72 gene, such as a C9orf72 gene having an expanded GGGGCC (G+C2) repeat (SEQ ID NO: 100). The C9orf72 RNA transcript may be within a cell, e.g., a cell within a subject, such as a human. The use of these iRNAs enables the targeted degradation of one or more RNAs of the corresponding gene (C9orf72 gene) in mammals. The iRNAs of the invention have been designed to target a C9orf72 gene transcript, e.g., a C9orf72 gene transcript having an expanded GGGGCC hexanucleotide repeat (SEQ ID NO: 100) in an intron of the gene. The agents may target a mature C9orf72 mRNA (an mRNA having introns spliced out) or a sense or antisense C9orf72 RNA containing a hexanucleotide-repeat (e.g., an RNA containing C9orf72 intron 1A). The described iRNAs may have one or more nucleotide modifications or combination of nucleotide modifications that increase activity, delivery, and/or stability of the İRNAs.

The agents may target a sense strand of a mature C9orf72 mRNA (an mRNA having introns spliced out) or a sense or antisense strand of a C9orf72 RNA containing a hexanucleotide-repeat (an RNA containing C9orf72 intron 1A). In certain aspects of the invention, the RNAi agents of the disclosure may target a C9orf72 sense and/or antisense RNA transcript containing a hexanucleotide-repeat (an RNA containing C9orf72 intron 1A). Targeting a C9orf72 sense and/or antisense strand RNA containing a hexanucleotide-repeat can inhibit expression of, or reduce the presence of, aberrant dipeptide-repeat (DPR) proteins (poly(GA), poly(GR), poly(GP), poly(PA), and poly(PR)), which are produced from all reading frames of either sense or antisense repeat-containing C9orf72 RNAs through repeat-associated non-AUG-dependent (RAN) translation, in cells of the nervous systems of subjects having a C9orf72-associated disease. In some embodiments, a combination of an RNA agent targeting a C9orf72 sense strand RNA containing a hexanucleotide-repeat and an RNA agent targeting a C9orf72 antisense strand RNA containing a hexanucleotide-repeat are provided together.

The iRNAs of the invention may decrease the levels of C9orf72 mature mRNA less than they decrease the levels of C9orf72 RNA containing a hexanucleotide repeat. For example, the iRNAs of the invention may decrease the levels of the C9orf72 mature mRNA by no more than about 50%, and reduce the level of sense- and antisense-containing C9orf72 RNA foci, reduce the levels of one or more aberrant dipeptide-repeat (DPR) proteins (poly(GA), poly(GR), poly(GP), poly(PA), and poly(PR)), and/or decrease the levels of C9orf72 sense and/or antisense RNA containing a hexanucleotide-repeat by more than about 50%. Without intending to be limited by theory, it is believed that a combination or sub-combination of the foregoing properties and the specific target sites, or the specific modifications in these iRNAs confer to the iRNAs of the invention improved efficacy, stability, potency, durability, and safety.

In one aspect, the present invention provides double stranded ribonucleic acid (dsRNA) agents for knocking down a C9orf72 target RNA in a cell.

In one embodiment, the dsRNA agents target a region of a C9orf72 target RNA containing a hexanucleotide repeat, e.g., multiple contiguous copies of a GGGGCC (SEQ ID NO: 100) or CCCCGG hexanucleotide repeat. In some embodiments, the C9orf72 target RNA can be a sense C9orf72 RNA containing a hexanucleotide repeat, an antisense C9orf72 target RNA containing a hexanucleotide repeat, or a combination of a sense C9orf72 RNA containing a hexanucleotide repeat and an antisense C9orf72 target RNA containing a hexanucleotide repeat.

In one aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand comprises at least 15, e.g., 15, 16, 17, 18, 19, 20, 21, 22, or 23, contiguous nucleotides differing by no more than 3, e.g., 3, 2, 1, or 0, nucleotides from the nucleotide sequence of SEQ ID NO: 13 and the antisense strand comprises a nucleotide sequence comprising at least 15, e.g., 15, 16, 17, 18, 19, 20, 21, 22, or 23, contiguous nucleotides differing by no more than 3 nucleotides from the corresponding portion of the nucleotide sequence of SEQ ID NO: 14; and wherein the sense strand or the antisense strand or both the sense strand and the antisense strand comprises at least one modified nucleotide.

In one aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand comprises at least 15, e.g., 15, 16, 17, 18, 19, 20, 21, 22, or 23, contiguous nucleotides differing by no more than 3, e.g., 3, 2, 1, or 0, nucleotides from the nucleotide sequence of SEQ ID NO: 17, and the antisense strand comprises a nucleotide sequence comprising at least 15, e.g., 15, 16, 17, 18, 19, 20, 21, 22, or 23, contiguous nucleotides differing by no more than 3 nucleotides from the corresponding portion of the nucleotide sequence of SEQ ID NO: 18; and wherein the sense strand or the antisense strand or both the sense strand and the antisense strand comprises at least one modified nucleotide.

In one aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand comprises at least 15, e.g., 15, 16, 17, 18, 19, 20, 21, 22, or 23, contiguous nucleotides differing by no more than 3, e.g., 3, 2, 1, or 0, nucleotides from the nucleotide sequence of SEQ ID NO: 19, and the antisense strand comprises a nucleotide sequence comprising at least 15, e.g., 15, 16, 17, 18, 19, 20, 21, 22, or 23, contiguous nucleotides differing by no more than 3 nucleotides from the corresponding portion of the nucleotide sequence of SEQ ID NO:20; and wherein the sense strand or the antisense strand or both the sense strand and the antisense strand comprises at least one modified nucleotide.

In one embodiment, an RNAi agent of the disclosure targets a C9orf72 antisense RNA transcript in a region between the antisense RNA transcript start site and the 5′ end of exon 1B. In another embodiment, an RNAi agent of the disclosure targets a C9orf72 antisense RNA transcript in a region between the antisense RNA transcript start site and the hexanucleotide repeat. In another embodiment, an RNAi agent of the disclosure targets a C9orf72 antisense RNA transcript in a region between the antisense RNA transcript start site and the 3′ end of exon 1A. In another embodiment, an RNAi agent of the disclosure targets a C9orf72 antisense RNA transcript in a region between the antisense RNA transcript start site and the 5′ end of exon 1A. In another embodiment, an RNAi agent of the disclosure targets a C9orf72 antisense RNA transcript in a region between the antisense RNA transcript start site and 500 bases upstream of the 5′ end of exon 1A. In another embodiment, an RNAi agent of the disclosure targets a C9orf72 antisense RNA transcript in a region between the antisense RNA transcript start site and 1000 bases upstream of the 5′ end of exon 1A. In another embodiment, an RNAi agent of the disclosure targets a C9orf72 antisense RNA transcript in a region between the antisense RNA transcript start site and 1500 bases upstream of the 5′ end of exon 1A. In another embodiment, an RNAi agent of the disclosure targets a C9orf72 antisense RNA transcript in a region between the antisense RNA transcript start site and 2000 bases upstream of the 5′ end of exon 1A.

In another embodiment, an RNAi agent of the disclosure targets a C9orf72 antisense RNA transcript in a region between the 5′ end of exon 1B and the hexanucleotide repeat. In another embodiment, an RNAi agent of the disclosure targets a C9orf72 antisense RNA transcript in a region between the 5′ end of exon 1B and the 3′ end of exon 1A. In another embodiment, an RNAi agent of the disclosure targets a C9orf72 antisense RNA transcript in a region between the 5′ end of exon 1B and the 5′ end of exon 1A. In another embodiment, an RNAi agent of the disclosure targets a C9orf72 antisense RNA transcript in a region between the 5′ end of exon 1B and 500 bases upstream of the 5′ end of exon 1A. In another embodiment, an RNAi agent of the disclosure targets a C9orf72 antisense RNA transcript in a region between the 5′ end of exon 1B and 1000 bases upstream of the 5′ end of exon 1A. In another embodiment, an RNAi agent of the disclosure targets a C9orf72 antisense RNA transcript in a region between the 5′ end of exon 1B and 1500 bases upstream of the 5′ end of exon 1A. In another embodiment, an RNAi agent of the disclosure targets a C9orf72 antisense RNA transcript in a region between the 5′ end of exon 1B and 2000 bases upstream of the 5′ end of exon 1A.

In another embodiment, an RNAi agent of the disclosure targets a C9orf72 antisense RNA transcript in a region between the hexanucleotide repeat and the 3′ end of exon 1A. In another embodiment, an RNAi agent of the disclosure targets a C9orf72 antisense RNA transcript in a region between the hexanucleotide repeat and the 5′ end of exon 1A. In another embodiment, an RNAi agent of the disclosure targets a C9orf72 antisense RNA transcript in a region between the hexanucleotide repeat and 500 bases upstream of the 5′ end of exon 1A. In another embodiment, an RNAi agent of the disclosure targets a C9orf72 antisense RNA transcript in a region between the hexanucleotide repeat and 1000 bases upstream of the 5′ end of exon 1A. In another embodiment, an RNAi agent of the disclosure targets a C9orf72 antisense RNA transcript in a region between the hexanucleotide repeat and 1500 bases upstream of the 5′ end of exon 1A. In another embodiment, an RNAi agent of the disclosure targets a C9orf72 antisense RNA transcript in a region between the hexanucleotide repeat and 2000 bases upstream of the 5′ end of exon 1A.

In one aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand comprises a nucleotide sequence comprising at least 15, e.g., 15, 16, 17, 18, 19, 20, 21, 22, or 23, contiguous nucleotides differing by no more than 3 nucleotides from an mRNA target sequence of any one of Tables 4A-4G and 7A-7E; and the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the corresponding mRNA target sequence of any one of Tables 4A-4G and 7A-7E.

In certain embodiments, the sense strand or the antisense strand or both the sense strand and the antisense strand is conjugated to one or more lipophilic moieties.

In one aspect, the present invention provides a combination of:
a) a double stranded ribonucleic acid (dsRNA) agent for inhibiting expression of C9orf72. wherein the dsRNA agent comprises a sense strand and an antisense strand forming a double stranded region, wherein the sense strand comprises a nucleotide sequence comprising at least 15, e.g., 15, 16, 17, 18, 19, 20, 21, 22, or 23, contiguous nucleotides differing by no more than 3 nucleotides from an mRNA target sequence of any one of Tables 4A-4G; and the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the corresponding mRNA target sequence of any one of Tables 4A-4G; and
b) a double stranded ribonucleic acid (dsRNA) agent for inhibiting expression of C9orf72. wherein the dsRNA agent comprises a sense strand and an antisense strand forming a double stranded region, wherein the sense strand comprises a nucleotide sequence comprising at least 15, e.g., 15, 16, 17, 18, 19, 20, 21, 22, or 23, contiguous nucleotides differing by no more than 3 nucleotides from an mRNA target sequence of any one of Tables 7A-7E; and the antisense strand comprises at least 15. e.g., 15, 16, 17, 18, 19, 20, 21, 22, or 23, contiguous nucleotides differing by no more than 3, e.g., 3, 2, 1, or 0, nucleotides from the complement of the corresponding mRNA target sequence of any one of Tables 7A-7E.

In certain embodiments, the sense strand or the antisense strand or both the sense strand and the antisense strand is conjugated to one or more lipophilic moieties.

In one aspect, the present invention provides a combination of:
a) a dsRNA agent for inhibiting expression of a C9orf72 antisense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 21; and
b) a dsRNA agent for inhibiting expression of a C9orf72 sense strand transcript, wherein the dsRNA agent comprises a sense strand and an antisense strand forming a double stranded region. wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 7A-7E.

In one aspect, the present invention provides a combination of:
a) a dsRNA agent for inhibiting expression of a C9orf72 antisense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 22; and
b) a dsRNA agent for inhibiting expression of a C9orf72 sense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 7A-7E.

In one aspect, the present invention provides a combination of:
a) a dsRNA agent for inhibiting expression of a C9orf72 antisense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 23; and
b) a dsRNA agent for inhibiting expression of a C9orf72 sense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 7A-7E.

In one aspect, the present invention provides a combination of:
a) a dsRNA agent for inhibiting expression of a C9orf72 antisense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 24; and
b) a dsRNA agent for inhibiting expression of a C9orf72 sense strand transcript, wherein the dsRNA agent comprises a sense strand and an antisense strand forming a double stranded region. wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 7A-7E.

In one aspect, the present invention provides a combination of:
a) a dsRNA agent for inhibiting expression of a C9orf72 antisense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 25; and
b) a dsRNA agent for inhibiting expression of a C9orf72 sense strand transcript, wherein the dsRNA agent comprises a sense strand and an antisense strand forming a double stranded region. wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 7A-7E.

In one aspect, the present invention provides a combination of:
a) a dsRNA agent for inhibiting expression of a C9orf72 antisense strand transcript, wherein the dsRNA agent comprises a sense strand and an antisense strand forming a double stranded region. wherein the antisense strand comprises a nucleotide sequence comprising at least 15 contiguous nucleotides differing by no more than 3 nucleotides from the complement of SEQ ID NO: 26; and
b) a dsRNA agent for inhibiting expression of a C9orf72 sense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 7A-7E.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 51; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 52; and
b) a dsRNA agent for inhibiting expression of a C9orf72 antisense strand transcript, wherein the dsRNA agent comprises a sense strand and an antisense strand forming a double stranded region. wherein the antisense strand comprises at least 15, e.g., 15, 16, 17, 18, 19, 20, 21, 22, or 23, contiguous nucleotides differing by no more than 3, e.g., 3, 2. 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region. wherein the antisense strand comprises a nucleotide sequence comprising at least 15 contiguous nucleotides differing by no more than 3 nucleotides from the complement of SEQ ID NO: 53; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 54; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region. wherein the antisense strand comprises a nucleotide sequence comprising at least 15 contiguous nucleotides differing by no more than 3 nucleotides from the complement of SEQ ID NO: 55; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4a-4g.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 56; and
b) a dsRNA agent for inhibiting expression of a C9orf72 antisense strand transcript, wherein the dsRNA agent comprises a sense strand and an antisense strand forming a double stranded region. wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region. wherein the antisense strand comprises a nucleotide sequence comprising at least 15 contiguous nucleotides differing by no more than 3 nucleotides from the complement of SEQ ID NO: 57; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 58; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 59; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 60; and
b) a dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of:
a) a dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 61; and
b) dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2. 1, or 0, nucleotides from the complement of the any of the mRNA target sequence of any one of Tables 4A-4G.

In one aspect, the present invention provides a combination of:
a) dsRNA agent for inhibiting expression of a C9orf72 sense strand transcript, wherein the dsRNA agent comprises a sense strand and an antisense strand forming a double stranded region, wherein the antisense strand comprises a nucleotide sequence comprising at least 15 contiguous nucleotides differing by no more than 3 nucleotides from the complement of SEQ ID NO: 62; and
b) dsRNA agent for inhibiting expression of a C9orf72 antisense strand transcript, wherein the dsRNA agent comprises a sense strand and an antisense strand forming a double stranded region, wherein the antisense strand comprises at least 15, e.g., 15, 16, 17, 18, 19, 20, 21, 22, or 23, contiguous nucleotides differing by no more than 3, e.g., 3, 2, 1, or 0, nucleotides from the complement of the any of the mRNA target sequence of any one of Tables 4A-4G.

In one aspect, the present invention provides a combination of a first dsRNA agent targeting a C9orf72 antisense RNA transcript and a second dsRNA agent targeting a C9orf72 sense strand transcript wherein,
a) the first dsRNA agent comprises an antisense strand comprising a nucleotide sequence comprising at least 15 contiguous nucleotides differing by no more than 3 nucleotides from the antisense sequence of AD-1446213; and the second dsRNA agent comprises an antisense strand comprising a nucleotide sequence comprising at least 15 contiguous nucleotides differing by no more than 3 nucleotides from the antisense sequence of AD-1285238;
b) the first dsRNA agent comprises an antisense strand comprising a nucleotide sequence comprising at least 15 contiguous nucleotides differing by no more than 3 nucleotides from the antisense sequence of AD-1446213; and the second dsRNA agent comprises an antisense strand comprising a nucleotide sequence comprising at least 15 contiguous nucleotides differing by no more than 3 nucleotides from the antisense sequence of AD-1285234;
c) the first dsRNA agent comprises an antisense strand comprising a nucleotide sequence comprising at least 15 contiguous nucleotides differing by no more than 3 nucleotides from the antisense sequence of AD-1446246; and the second dsRNA agent comprises an antisense strand comprising a nucleotide sequence comprising at least 15 contiguous nucleotides differing by no more than 3 nucleotides from the antisense sequence of AD-1285238;
d) the first dsRNA agent comprises an antisense strand comprising a nucleotide sequence comprising at least 15 contiguous nucleotides differing by no more than 3 nucleotides from the antisense sequence of AD-1446246; and the second dsRNA agent comprises an antisense strand comprising a nucleotide sequence comprising at least 15 contiguous nucleotides differing by no more than 3 nucleotides from the antisense sequence of AD-1285234;
e) the first dsRNA agent comprises an antisense strand comprising a nucleotide sequence comprising at least 15 contiguous nucleotides differing by no more than 3 nucleotides from the antisense sequence of AD-1446268; and the second dsRNA agent comprises an antisense strand comprising a nucleotide sequence comprising at least 15 contiguous nucleotides differing by no more than 3 nucleotides from the antisense sequence of AD-1285238;
f) the first dsRNA agent comprises an antisense strand comprising a nucleotide sequence comprising at least 15 contiguous nucleotides differing by no more than 3 nucleotides from the antisense sequence of AD-1446268; and the second dsRNA agent comprises an antisense strand comprising a nucleotide sequence comprising at least 15 contiguous nucleotides differing by no more than 3 nucleotides from the antisense sequence of AD-1285234.

In another aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand or the antisense strand is a sense strand or an antisense strand selected from the group consisting of any of the sense strands and antisense strands in any one of Tables 2, 3, 10A, 10C, 11, and 12; and wherein the sense strand, the antisense strand, or both the sense strand and the antisense strand comprises at least one modified nucleotide.

In another aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand comprises at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13; and wherein the sense strand, the antisense strand, or both the sense strand and the antisense strand comprises at least one modified nucleotide.

In one embodiment, the sense strand or the antisense strand is a sense strand or an antisense strand selected from the sense strand or antisense strand of a duplex selected from the group consisting of AD-1446213.1; AD-1446217.1; AD-1446222.1; AD-1446234.1; AD-1446243.1; AD-1446246.1; AD-1446252.1; AD-1446259.1; AD-1446265.1; AD-1446268.1; AD-1446271.1; AD-1446279.1; AD-1446289.1; and AD-1446294.1.

In one embodiment, the sense strand or the antisense strand is a sense strand or an antisense strand selected from the sense strand or antisense strand of a duplex selected from the group consisting of AD-1446213.1; AD-1446246.1; and AD-1446268.1.

In one aspect, the present invention provides a double stranded ribonucleic acid (dsRNA) agent for inhibiting expression of C9orf72, wherein the dsRNA agent comprises a sense strand and an antisense strand forming a double stranded region, wherein the antisense strand comprises at least 15. e.g., 15, 16, 17, 18, 19, 20, 21, 22, or 23, contiguous nucleotides differing by no more than 3, e.g., 3, 2, 1, or 0, nucleotides from any one of the antisense nucleotide sequences in any one of Tables 5, 6, 10B, and 10D; and wherein the sense strand, the antisense strand, or both the sense strand and the antisense strand comprises at least one modified nucleotide.

In another aspect, the present invention provides a double stranded ribonucleic acid (dsRNA) for inhibiting expression of c9orf72, wherein said dsRNA comprises a sense strand and an antisense strand forming a double stranded region, wherein the sense strand comprises at least 15, e.g., 15, 16, 17, 18, 19, 20, or 21, contiguous nucleotides differing by no more than three, e.g., 3, 2, 1, or 0, nucleotides from any one of the nucleotide sequence of nucleotides 1-23; 15-37; 33-55; 37-59; 59-81; 62-84; or 69-91 of SEQ ID NO: 1, and the antisense strand comprises at least 15, e.g., 15, 16, 17, 18, 19, 20, 21, 22, or 23, contiguous nucleotides from the corresponding nucleotide sequence of SEQ ID NO:5; and wherein the sense strand, the antisense strand, or both the sense strand and the antisense strand comprises at least one modified nucleotide.

In one embodiment, the antisense strand comprises at least 15, e.g., 15, 16, 17, 18, 19, 20, 21, 22, or 23, contiguous nucleotides differing by no more than three, e.g., 3, 2, 1, or 0, nucleotides from any one of the antisense strand nucleotide sequences of a duplex selected from the group consisting of AD-1446073.1; AD-1446075.1; AD-1285246.2; AD-1446084.1; AD-1446087.1; AD-1446090.1; and AD-1446095.1.

In one aspect, the present invention provides a double stranded ribonucleic acid (dsRNA) for inhibiting expression of c9orf72, wherein said dsRNA comprises a sense strand and an antisense strand forming a double stranded region, wherein the sense strand comprises at least 15, e.g., 15, 16, 17, 18, 19, 20, or 21, contiguous nucleotides differing by no more than three, e.g., 3, 2, 1, or 0, nucleotides from any one of the nucleotide sequence of nucleotides 5197-5219; 5213-5235; 5223-5245; 5226-5248; 5227-5249; 5228-5250, 5229-5251, 5230-5252, 5231-5253, 5233-5255; 5235-5256, 5241-5263; 5245-5267; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and the antisense strand comprises at least 15, e.g., 15, 16, 17, 18, 19, 20, 21, 22, or 23, contiguous nucleotides from the corresponding nucleotide sequence of SEQ ID NO: 16; and wherein the sense strand, the antisense strand, or both the sense strand and the antisense strand comprises at least one modified nucleotide.

In one embodiment, the antisense strand comprises at least 15, e.g., 15, 16, 17, 18, 19, 20, 21, 22, or 23, contiguous nucleotides differing by no more than three, e.g., 3, 2, 1, or 0, nucleotides from any one of the antisense strand nucleotide sequences of a duplex selected from the group consisting of AD-1285231.1. AD-1285232.1. AD-1285233.1. AD-1285235.1. AD-1285237.1. AD-1285239.1. AD-1285240.1. AD-1285242.1, AD-1285244.1; AD-1285238.1; AD-1285243.1; AD-1285234.1; AD-1285241.1; AD-1285236.1; AD-1446111.1; AD-1446117.1; AD-1446147.1; AD-1446157.1; AD-1446168.1; AD-1446180.1; AD-1446189.1; AD-1446196.1; AD-1446202.1; AD-1446205.1.

In one embodiment, the antisense strand comprises at least 15, e.g., 15, 16, 17, 18, 19, 20, 21, 22, or 23, contiguous nucleotides differing by no more than three, e.g., 3, 2, 1, or 0, nucleotides from any one of the antisense strand nucleotide sequences of a duplex selected from the group consisting of AD-1285231.1. AD-1285232.1, AD-1285233.1. AD-1285234.1. AD-1285235.1, AD-1285236.1, AD-1285237.1. AD-1285239.1. AD-1285240.1, AD-1285241.1, AD-1285242.1, AD-1285243.1, AD-1446087.1, and AD-1446090.1.

In one embodiment, the antisense strand comprises at least 15, e.g., 15, 16, 17, 18, 19, 20, 21, 22, or 23, contiguous nucleotides differing by no more than three, e.g., 3, 2. 1, or 0, nucleotides from any one of the antisense strand nucleotide sequences of a duplex selected from the group consisting of AD-1285238.1 and AD-1285234.1.

In one aspect, the present invention provides a double stranded ribonucleic acid (dsRNA) for inhibiting expression of c9orf72, wherein the dsRNA comprises a sense strand and an antisense strand forming a double stranded region, wherein the sense strand comprises at least 15, e.g., 15, 16, 17, 18, 19, 20, or 21, contiguous nucleotides differing by no more than three, e.g., 3, 2, 1, or 0, nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and the antisense strand comprises at least 15, e.g., 15, 16, 17, 18, 19, 20, 21, 22, or 23, contiguous nucleotides from the corresponding nucleotide sequence of SEQ ID NO: 16; and wherein the sense strand, the antisense strand, or both the sense strand and the antisense strand comprises at least one modified nucleotide.

In one aspect, the present invention provides a double stranded ribonucleic acid (dsRNA) for inhibiting expression of c9orf72, wherein the dsRNA comprises a sense strand and an antisense strand forming a double stranded region, wherein the sense strand comprises at least 15, e.g., 15, 16, 17, 18, 19, 20, or 21, contiguous nucleotides differing by no more than three, e.g., 3, 2, 1, or 0, nucleotides from any one of the nucleotide sequence of nucleotides 15-52; 17-40; 32-59; 32-55; 35-59; 36-59; 58-87; 59-87; 59-84; or 64-87 of SEQ ID NO: 1, and the antisense strand comprises at least 15, e.g., 15, 16, 17, 18, 19, 20, 21, 22, or 23, contiguous nucleotides from the corresponding nucleotide sequence of SEQ ID NO:5; and wherein the sense strand, the antisense strand, or both the sense strand and the antisense strand comprises at least one modified nucleotide.

In one aspect, the present invention provides a double stranded ribonucleic acid (dsRNA) for inhibiting expression of c9orf72, wherein the dsRNA comprises a sense strand and an antisense strand forming a double stranded region, wherein the sense strand comprises at least 15, e.g., 15, 16, 17, 18, 19, 20, or 21, contiguous nucleotides differing by no more than three, e.g., 3, 2, 1, or 0, nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and the antisense strand comprises at least 15, e.g., 15, 16, 17, 18, 19, 20, 21, 22, or 23, contiguous nucleotides from the corresponding nucleotide sequence of SEQ ID NO: 14; and wherein the sense strand, the antisense strand, or both the sense strand and the antisense strand comprises at least one modified nucleotide.

In one aspect, the present invention provides a double stranded ribonucleic acid (dsRNA) agent for inhibiting expression of C9orf72, wherein the dsRNA agent comprises a sense strand and an antisense strand forming a double stranded region, wherein the sense strand or the antisense strand is a sense strand or an antisense strand selected from the group consisting of any of the sense strands and antisense strands in any one of Table 8 and 9; and wherein the sense strand, the antisense strand, or both the sense strand and the antisense strand comprises at least one modified nucleotide.

In one embodiment, the sense strand, the antisense strand, or both the sense strand and the antisense strand is conjugated to one or more lipophilic moieties.

In one embodiment, the lipophilic moiety is conjugated to one or more internal positions in the double stranded region of the dsRNA agent.

In one embodiment, the lipophilic moiety is conjugated via a linker or carrier.

In one embodiment, the lipophilicity of the lipophilic moiety, measured by logKow, exceeds 0.

In one embodiment, the hydrophobicity of the double-stranded RNAi agent, measured by the unbound fraction in a plasma protein binding assay of the double-stranded RNAi agent, exceeds 0.2.

In one embodiment, the plasma protein binding assay is an electrophoretic mobility shift assay using human serum albumin protein.

In some embodiments, the dsRNA agent comprises at least one modified nucleotide.

In one embodiment, no more than five of the sense strand nucleotides and no more than five of the nucleotides of the antisense strand are unmodified nucleotides

In one embodiment, all of the nucleotides of the sense strand are modified nucleotides. In one embodiment, all of the nucleotides of the antisense strand are modified nucleotides. In one embodiment, all of the nucleotides of the sense strand and all of the nucleotides of the antisense strand are modified nucleotides.

In one embodiment, at least one of the modified nucleotide is selected from the group consisting of: a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a 2′-O-hexadecyl modified nucleotide, a 2′-phosphate modified nucleotide, a 2′-5′-linked ribonucleotide (3′-RNA), a locked nucleotide, an unlocked nucleotide, a conformationally restricted nucleotide, a constrained ethyl nucleotide, an abasic nucleotide, an inverted abasic residue, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a 2′,3′-seco-modified nucleotide, a morpholino nucleotide, a phosphoramidate, a non-natural base comprising nucleotide, a tetrahydropyran modified nucleotide, a 1.5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a glycol modified nucleic acid (GNA), a nucleotide comprising glycol nucleic acid (GNA), a nucleotide comprising glycol nucleic acid S-Isomer (S-GNA), a nucleotide comprising 2-hydroxymethyl-tetrahydrofurane-5-phosphate, a nucleotide comprising 2′-deoxythymidine-3′-phosphate, a nucleotide comprising 2′-deoxyguanosine-3′-phosphate, and a terminal nucleotide linked to a cholesteryl derivative and a dodecanoic acid bisdecylamide group; and combinations thereof.

In one embodiment, the modified nucleotide is selected from the group consisting of a 2′-deoxy-2′-fluoro modified nucleotide, a 2′-deoxy-modified nucleotide, 3′-terminal deoxy-thymine nucleotides (dT), a 2′-O-hexadecyl modified nucleotide, a 2′-phosphate modified nucleotide, a glycol modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In one embodiment, the modified nucleotide comprises a short sequence of 3′-terminal deoxy-thymine nucleotides (dT).

In one embodiment, the modified nucleotides are independently selected from the group consisting of: 2′-O-methyl modified nucleotide, GNA modified nucleotides, and 2′fluoro modified nucleotides, 2′-phosphate modified nucleotide, 2′-O-hexadecyl modified nucleotide, and 2′-phosphate modified nucleotide.

In one embodiment, substantially all of the modified nucleotides of the sense strand are selected from the group consisting of 2′-O-methyl modified nucleotides and 2′-fluoro modified nucleotides. In some embodiments, all of the modified nucleotides of the sense strand are selected from the group consisting of 2′-O-methyl modified nucleotides and 2′-fluoro modified nucleotides.

In one embodiment, substantially all of the modified nucleotides of the antisense strand are selected from the group consisting of 2′-O-methyl modified nucleotides, 2′-phosphate modified nucleotides, glycol nucleic acid modified nucleotides and 2′-fluoro modified nucleotides. In some embodiments, all of the modified nucleotides of the antisense strand are selected from the group consisting of 2′-O-methyl modified nucleotides, 2′-phosphate modified nucleotides, glycol nucleic acid modified nucleotides and 2′-fluoro modified nucleotides.

In one embodiment, substantially all of the modified nucleotides of the sense strand are selected from the group consisting of 2′-O-methyl modified nucleotides, 2′-fluoro modified nucleotides, 2′-O-hexadecyl modified nucleotides, and a glycol nucleic acid (GNA) modified nucleotides. In some embodiments, all of the modified nucleotides of the sense strand are selected from the group consisting of 2′-O-methyl modified nucleotides, 2′-fluoro modified nucleotides, ′-O-hexadecyl modified nucleotides, and glycol nucleic acid (GNA) modified nucleotides.

In one embodiment, substantially all of the modified nucleotides of the antisense strand are selected from the group consisting of 2′-O-methyl modified nucleotides, 2′-fluoro modified nucleotides, 2′-phosphate modified nucleotides, and glycol nucleic acid (GNA) modified nucleotides. In some embodiments, all of the modified nucleotides of the antisense strand are selected from the group consisting of 2′-O-methyl modified nucleotides, 2′-fluoro modified nucleotides, 2′-phosphate modified nucleotides, and glycol nucleic acid (GNA) modified nucleotides.

In some embodiments, the dsRNA agent comprises at least one phosphorothioate internucleotide linkage.

In one embodiment, the dsRNA agent comprises 6-8 phosphorothioate internucleotide linkages.

In one embodiment, the sense strand comprises at least one phosphorothioate or methylphosphonate internucleotide linkage and the antisense strand comprises at least one phosphorothioate or methylphosphonate internucleotide linkage.

In one embodiment, the sense strand comprises at least two phosphorothioate or methylphosphonate internucleotide linkages.

In one embodiment, the antisense strand comprises at least two, at least three, or at least four phosphorothioate or methylphosphonate internucleotide linkages.

In one embodiment, the at least one phosphorothioate or methylphosphonate internucleotide linkage is at the 5′-terminus of one strand, at the 3′-terminus of one strand, or is at both the 5′-terminus and the 3′-terminus of one strand.

In one embodiment, the at least one phosphorothioate or methylphosphonate internucleotide linkage is at the 5′-terminus of the sense strand. In some embodiments, the sense strand comprises two phosphorothioate internucleotide linkages at the 5′-terminus. In some embodiments, the sense strand comprises one phosphorothioate internucleotide linkage at the 5′-terminus and one phosphorothioate internucleotide linkage at the 3′-terminus. In some embodiments, the sense strand comprises two phosphorothioate internucleotide linkages at the 5′-terminus and two phosphorothioate internucleotide linkages at the 3′-terminus.

In one embodiment, the at least one phosphorothioate or methylphosphonate internucleotide linkage is at both the 5′ terminus and the 3′ terminus of the antisense strand. In some embodiments, the antisense strand comprises two phosphorothioate internucleotide linkages at the 5′-terminus and two phosphorothioate internucleotide linkages at the 3′-terminus. In some embodiments, the antisense strand comprises two phosphorothioate internucleotide linkages at the 5′-terminus and 1 phosphorothioate internucleotide linkage at the 3′-terminus. In some embodiments, the antisense strand comprises three phosphorothioate internucleotide linkages at the 5′-terminus and one phosphorothioate internucleotide linkage at the 3′-terminus. In some embodiments, the antisense strand comprises three phosphorothioate internucleotide linkages at the 5′-terminus and two phosphorothioate internucleotide linkages at the 3′-terminus.

In one embodiment, all of the modified nucleotides of the sense strandare selected from the group consisting of 2′-O-methyl modified nucleotides, 2′-O-hexadecyl modified nucleotides, and 2′-fluoro modified nucleotides, all of the modified nucleotides of the antisense strand are selected from the group consisting of 2′-O-methyl modified nucleotides, 2′-phosphate modified nucleotides, glycol nucleic acid modified nucleotides, and 2′-fluoro modified nucleotides, the sense strand comprises two phosphorothioate internucleotide linkages at the 5′-terminus, and the antisense strand comprises two phosphorothioate internucleotide linkages at the 5′-terminus and two phosphorothioate internucleotide linkages or a vinyl-phosphonate at the 3′-terminus.

In one embodiment, the sense strand is no more than 30 nucleotides in length. In another embodiment, the antisense strand is no more than 30 nucleotides in length. In one embodiment, the sense strand and the antisense strand are each independently no more than 30 nucleotides in length.

In one embodiment, at least one strand comprises a 3′-overhang of at least 1 nucleotide. In another embodiment, at least one strand comprises a 3′-overhang of at least 2 nucleotides. In one embodiment, the antisense strand comprises the 3′-overhang.

The double stranded region may be 15-30 nucleotide pairs in length; 17-23 nucleotide pairs in length; 17-25 nucleotide pairs in length; 23-27 nucleotide pairs in length; 19-21 nucleotide pairs in length; 21-23 nucleotide pairs in length, or 17, 18, 19, 20, 21, 22, or 23 nucleotide pairs in length. In some embodiments, the double stranded region is 20 nucleotides in length. In some embodiments, the double stranded region is 21 nucleotides in length. The double stranded region may have 0, 1, 2, or 3 mismatches.

The sense strand and the antisense strand may each be independently 17-30 nucleotides, 17-25, 19-30 nucleotides; 19-25 nucleotides; 19-23 nucleotides; or 21-23 nucleotides in length, or 19, 20, 21, 22, or 23 nucleotides in length. In some embodiments, the sense strand is 20 nucleotides in length. In some embodiments, the antisense strand is 22 nucleotides in length. In some embodiments, the sense strand is 23 nucleotides in length. In some embodiments, the antisense strand is 21 nucleotides in length. In some embodiments, the sense strand is 23 nucleotides in length and the antisense strand is 21 nucleotides in length. In some embodiments the sense strand is 23 nucleotides in length and contains inverted abasic residues at the 3′ and 5′ terminal nucleotide positions.

In one embodiment, the region of complementarity is at least 17 nucleotides in length. In other embodiments, the region of complementarity is 19-30 nucleotides in length; 19-25 nucleotides in length; or 21-23 nucleotides in length.

In one embodiment, the region of complementarity is at least 85% complementary to a sequence between the start of exon 1A and the start of exon 2 of the C9orf72 gene. In some embodiments, the antisense strand comprises a sequence of 15-25 contiguous nucleotides having at least 85% complementarity to a sequence of 15-25 contiguous nucleotides present in the sequence between the start of exon 1A and the start of exon 2 of the C9orf72 target RNA. In other embodiments, the region of complementarity is at least 90% complementary to a sequence between the start of exon 1A and the start of exon 2 of the C9orf72 target RNA. In one embodiment, the region of complementarity is at least 95% complementary to a sequence between the start of exon 1A and the start of exon 2 of the C9orf72 target RNA. In some embodiments, the region of complementarity is 100% complementary to a sequence between the start of exon 1A and the start of exon 2 of the C9orf72 target RNA. In some embodiments, the region of complementarity is 100% complementary to a sequence between the end of exon 1A and the start of the hexanucleotide repeat region of the C9orf72 target RNA.

In one embodiment, the region of complementarity is at least 85% complementary to a sequence between the end of exon 1A and the start of hexanucleotide repeat in intron 1A of the C9orf72 gene. In some embodiments, the antisense strand comprises a sequence of 15-25 contiguous nucleotides having at least 85% complementarity to a sequence of 15-25 contiguous nucleotides present in the sequence between the end of exon 1A and the start of hexanucleotide repeat in intron 1A of the C9orf72 target RNA. In other embodiments, the region of complementarity is at least 90% complementary to a sequence between the end of exon 1A and the start of hexanucleotide repeat in intron 1A of the C9orf72 target RNA. In one embodiment, the region of complementarity is at least 95% complementary to a sequence between the end of exon 1A and the start of hexanucleotide repeat in intron 1A of the C9orf72 target RNA. In some embodiments, the region of complementarity is 100% complementary to a sequence between the end of exon 1A and the start of hexanucleotide repeat in intron 1A of the C9orf72 target RNA.

In some embodiments of the compositions and methods of the invention, an RNAi agent further comprises one or more lipophilic moieties. The lipophilic moiety conjugated RNAi agents are advantageous for the in vivo delivery of nucleic acids, as well as compositions suitable for in vivo therapeutic use, as described herein. In one embodiment, one or more lipophilic moieties are conjugated to one or more internal positions on at least one strand. The lipophilic moiety can be conjugated to the internal positions via a linker or carrier. In some embodiments, the lipophilic moiety facilitates or improves delivery of the RNAi agent to a neuronal cell or a cell in a neuronal tissue.

In one embodiment, the internal position can be any position except the terminal two positions from each end of the at least one strand.

In another embodiment, the internal position can be any position except the terminal three positions from each end of the at least one strand.

In one embodiment, the internal position excludes a cleavage site region of the sense strand.

In one embodiment, the internal position can be any position except positions 9-12, counting from the 5′-end of the sense strand.

In another embodiment, the internal position can be any position except positions 11-13, counting from the 3′-end of the sense strand.

In one embodiment, the internal position excludes a cleavage site region of the antisense strand.

In one embodiment, the internal position can be any position except positions 12-14, counting from the 5′-end of the antisense strand.

In one embodiment, the internal position can be any position except positions 11-13 on the sense strand, counting from the 3′-end, and positions 12-14 on the antisense strand, counting from the 5′-end.

In one embodiment, the one or more lipophilic moieties are conjugated to one or more of the internal positions selected from the group consisting of positions 4-8 and 13-18 on the sense strand, and positions 6-10 and 15-18 on the antisense strand, counting from the 5′ end of each strand.

In another embodiment, the one or more lipophilic moieties are conjugated to one or more of the internal positions selected from the group consisting of positions 5, 6, 7, 15, and 17 on the sense strand, and positions 15 and 17 on the antisense strand, counting from the 5′-end of each strand.

In one embodiment, the internal positions in the double stranded region exclude a cleavage site region of the sense strand.

In one embodiment, the sense strand is 21 nucleotides in length, the antisense strand is 23 nucleotides in length, and the lipophilic moiety is conjugated to position 21, position 20, position 15, position 1, position 7, position 6, or position 2 of the sense strand or position 16 of the antisense strand, counting from the 5′-end.

In one embodiment, the lipophilic moiety is conjugated to position 21, position 20, position 15, position 1, or position 7 of the sense strand.

In another embodiment, the lipophilic moiety is conjugated to position 21, position 20, or position 15 of the sense strand, counting from the 5′-end.

In yet another embodiment, the lipophilic moiety is conjugated to position 20 or position 15 of the sense strand, counting from the 5′-end.

In one embodiment, the lipophilic moiety is conjugated to position 16 of the antisense strand, counting from the 5′-end.

In one embodiment, the lipophilic moiety is an aliphatic, alicyclic, or polyalicyclic compound. In one embodiment, the lipophilic moiety is selected from the group consisting of lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1.3-propanediol, heptadecyl group, palmitic acid, myristic acid, 03-(oleoyl)lithocholic acid, O3-(oleoyl)cholenic acid, dimethoxytrityl, or phenoxazine.

In one embodiment, the lipophilic moiety contains a saturated or unsaturated C4-C30 hydrocarbon chain, and an optional functional group selected from the group consisting of hydroxyl, amine, carboxylic acid, sulfonate, phosphate, thiol, azide, alkenyl, and alkynyl. In one embodiment, the the lipophilic moiety contains a C6-C30 alkyl, a C6-C30 alkenyl, or a C6-C30 alkynyl.

In one embodiment, the lipophilic moiety contains a saturated or unsaturated C6-C18 hydrocarbon chain. In one embodiment, the lipophilic moiety contains a saturated or unsaturated C6, C7. C8, C9, C10, C11, C12, C13, C15, C15, C16, C17, or C18 hydrocarbon chain. An unsaturated C6-C18 can be a monounsaturated C6-C18 or a polyunsaturated C6-C18.

In one embodiment, the lipophilic moiety contains a saturated or unsaturated C16 hydrocarbon chain. In one embodiment, the lipophilic moiety contains a C16 alkyl, a C16 alkenyl, or a C16 alkynyl. An unsaturated C16 can be a monounsaturated C16 or a polyunsaturated C16.

In one embodiment, the saturated or unsaturated C16 hydrocarbon chain is conjugated to position 6, counting from the 5′-end of the strand.

In one embodiment, the lipophilic moiety is conjugated via a carrier that replaces one or more nucleotide(s) in the internal position(s) or the double stranded region.

In one embodiment, the carrier is a cyclic group selected from the group consisting of pyrrolidinyl, pyrazolinyl, pyrazolidinyl, imidazolinyl, imidazolidinyl, piperidinyl, piperazinyl, [1.3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone.

In one embodiment, the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction, or carbamate.

In one embodiment, the lipophilic moiety is conjugated to a nucleobase, sugar moiety, or internucleosidic linkage.

In one embodiment, the lipophilic moiety or targeting ligand is conjugated via a bio-cleavable linker selected from the group consisting of DNA, RNA, disulfide, amide, functionalized monosaccharides or oligosaccharides of galactosamine, glucosamine, glucose, galactose, mannose, and combinations thereof.

In one embodiment, the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the dsRNA agent further comprises a tareting ligand that targets a neuronal cell, a cell in a neuronal tissue, or a cell in a central nervous system tissue.

In one embodiment, the dsRNA agent further comprises a targeting ligand that targets a liver tissue.

In one embodiment, the targeting ligand is a GalNAc conjugate.

In one embodiment, the dsRNA agent further comprises a terminal, chiral modification occurring at the first internucleotide linkage at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp configuration or Sp configuration.

In another embodiment, the dsRNA agent further comprises a terminal, chiral modification occurring at the first and second internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In yet another embodiment, the dsRNA agent further comprises a terminal, chiral modification occurring at the first, second and third internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In another embodiment, the dsRNA agent further comprises a terminal, chiral modification occurring at the first, and second internucleotide linkages at the 3′ end of the antisense strand, having the linkage phosphorus atom in Sp configuration, a terminal, chiral modification occurring at the third internucleotide linkages at the 3′ end of the antisense strand, having the linkage phosphorus atom in Rp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In another embodiment, the dsRNA agent further comprises a terminal, chiral modification occurring at the first, and second internucleotide linkages at the 3′ end of the antisense strand, having the linkage phosphorus atom in Sp configuration, a terminal, chiral modification occurring at the first, and second internucleotide linkages at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In one embodiment, the dsRNA agent further comprises a phosphate or phosphate mimic at the 5′-end of the antisense strand.

In one embodiment, the phosphate mimic is a 5′-vinyl phosphonate (VP).

In one embodiment, the base pair at the 1 position of the 5′-end of the antisense strand of the duplex is an AU base pair.

In one embodiment, the sense strand has a total of 21 nucleotides and the antisense strand has a total of 23 nucleotides.

In one embodiment, the dsRNA agent inhibits expression of the C9orf72 target RNA comprising the hexanucleotide repeat by at least 10%, at least 20%, at least 30%, at least 40%, at least 50%, or at least 60% within 24-48 hours after administration to a cell expressing the C9orf72 target RNA comprising the hexanucleotide repeat.

In one embodiment, the dsRNA agent selectively inhibits expression of the C9orf72 target RNA comprising the hexanucleotide repeat relative to expression of a mature C9orf72 messenger RNA.

In one embodiment, the dsRNA agent inhibits expression of the mature C9orf72 messenger RNA by less than 50%, less than 40%, less than 30%, less than 20%, or less than 10% within 24-48 hours after administration to a cell expressing the mature C9orf72 messenger RNA.

In one embodiment, the dsRNA agent reduces (poly(GA), poly(GR), poly(GP), poly(PA), and poly(PR) dipeptide repeat protein synthesis within 24-48 hours after administration to a cell expressing the C9orf72 target RNA comprising the hexanucleotide repeat. In some embodiments, the dsRNA agent reduces dipeptide repeat protein synthesis by at least 10%, at least 20%, at least 30%, at least 40%, at least 50%, at least 60%, at least 70%, or at least 80% within 24-48 hours after administration to the cell.

The present invention also provides cells and pharmaceutical compositions for inhibiting expression of a gene encoding C9orf72 comprising the dsRNA agents of the invention, such.

In one embodiment, the dsRNA agent is in an unbuffered solution, such as saline or water.

In another embodiment, the dsRNA agent is in a buffer solution, such as a buffer solution comprising acetate, citrate, prolamine, carbonate, or phosphate or any combination thereof; or phosphate buffered saline (PBS).

The present invention further provides a composition comprising two or more, e.g., 2, 3, or 4, dsRNA agents for inhibiting expression of C9orf72.

In one embodiment, the composition comprises a first dsRNA agent targeting a sense strand of C9orf72 (an exon or intron of C9orf72) and a seond dsRNA agent targeting an antisense strand of C9orf72 (an exon or intron of C9orf72).

In some embodiments, suitable agents targeting a sense strand of C9orf72 for use in the compositions of the invention comprising two or more dsRNA agents comprise a sense strand and an antisense strand, forming a double stranded region, and selected from the group consisting of
a) a sense strand comprising at least 15 contiguous nucleotides differing by no more than 3 nucleotides from the nucleotide sequence of SEQ ID NO:1 and an antisense strand comprising a nucleotide sequence comprising at least 15 contiguous nucleotides differing by no more than 3 nucleotides from the corresponding portion of the nucleotide sequence of SEQ ID NO:5,
b) a sense strand comprising at least 15 contiguous nucleotides differing by no more than 3 nucleotides from the nucleotide sequence of SEQ ID NO:15 and an antisense strand comprising a nucleotide sequence comprising at least 15 contiguous nucleotides differing by no more than 3 nucleotides from the corresponding portion of the nucleotide sequence of SEQ ID NO:16,
c) an antisense strand comprising at least 15 contiguous nucleotides differing by no more than 3 nucleotides from any one of the antisense nucleotide sequences in any one of Tables 5, 6, 10B, and 10D; and wherein the sense strand, the antisense strand, or both the sense strand and the antisense strand is conjugated to one or more lipophilic moieties;
d) a sense strand comprising at least 15 contiguous nucleotides differing by no more than three nucleotides from any one of the nucleotide sequence of nucleotides 1-23; 15-37; 33-55; 37-59; 59-81; 62-84, or 69-91 of SEQ ID NO: 1, and an antisense strand comprising at least 15 contiguous nucleotides from the corresponding nucleotide sequence of SEQ ID NO:5;
e) a sense strand comprising at least 15 contiguous nucleotides differing by no more than three nucleotides from any one of the nucleotide sequence of nucleotides 5197-5219; 5213-5235; 5223-5245, 5226-5248; 5227-5249, 5228-5250, 5229-5251, 5230-5252, 5231-5253, 5233-5255; 5235-5256, 5241-5263; 5245-5267; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and an antisense strand comprising at least 15 contiguous nucleotides from the corresponding nucleotide sequence of SEQ ID NO:16;
f) a sense strand comprising at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and an antisense strand comprising at least 15 contiguous nucleotides from the corresponding nucleotide sequence of SEQ ID NO:16;
g) a sense strand comprising at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and an antisense strand comprising at least 15 contiguous nucleotides from the corresponding nucleotide sequence of SEQ ID NO:5; and
h) an antisense strand comprising at least 15 contiguous nucleotides differing by no more than 3 nucleotides from any one of the antisense nucleotide sequences in any one of Tables 8 and 9, wherein the sense strand, the antisense strand, or both the sense strand and the antisense strand comprises at least one modified nucleotide.

In certain embodiments, suitable agents targeting a sense strand of C9orf72, e.g., of a C9orf72 exon or intron sense sequence, for use in the compositions of the invention comprising two or more dsRNA agents such as those dsRNA agents disclosed in PCT Publication No. WO 2021/119226, the entire contents of which are incorporated herein by reference.

In certain embodiments, suitable agents targeting an antisense strand of C9orf72 for use in the compositions of the invention comprising two or more dsRNA agents comprise a sense strand and an antisense strand, forming a double stranded region, and selected from the group consisting of
a) a sense strand comprising at least 15 contiguous nucleotides differing by no more than 3 nucleotides from the nucleotide sequence of SEQ ID NO: 13 and an antisense strand comprising a nucleotide sequence comprising at least 15 contiguous nucleotides differing by no more than 3 nucleotides from the corresponding portion of the nucleotide sequence of SEQ ID NO:14,
b) a sense strand comprising at least 15 contiguous nucleotides differing by no more b) than 3 nucleotides from the nucleotide sequence of SEQ ID NO: 17 and an antisense strand comprising a nucleotide sequence comprising at least 15 contiguous nucleotides differing by no more than 3 nucleotides from the corresponding portion of the nucleotide sequence of SEQ ID NO:18,
c) a sense strand comprising at least 15 contiguous nucleotides differing by no more than 3 nucleotides from the nucleotide sequence of SEQ ID NO: 19 and an antisense strand comprising a nucleotide sequence comprising at least 15 contiguous nucleotides differing by no more than 3 nucleotides from the corresponding portion of the nucleotide sequence of SEQ ID NO:20,
d) an antisense comprising a nucleotide sequence selected from the group consisting of any of the antisense strand nucleotide sequences in any one of Tables 2, 3, 10A, 10C, 11, and 12; and
e) a sense strand comprising at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13;
f) a sense strand comprising at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and an antisense strand comprising at least 15 contiguous nucleotides from the corresponding nucleotide sequence of SEQ ID NO: 14,
wherein the sense strand, the antisense strand, or both the sense strand and the antisense strand comprises at least one modified nucleotide.

In one aspect, the present invention provides a composition comprising two or more double stranded ribonucleic acid (dsRNA) agents for inhibiting expression of C9orf72.
wherein each dsRNA agent independently comprises a sense strand and an antisense strand forming a double stranded region,
wherein a first dsRNA agent targeting the antisense strand of C9orf72 is selected from the group consisting of
a) a dsRNA agent comprising a sense strand comprising at least 15 contiguous nucleotides differing by no more than 3 nucleotides from the nucleotide sequence of SEQ ID NO: 13 and an antisense strand comprising a nucleotide sequence comprising at least 15 contiguous nucleotides differing by no more than 3 nucleotides from the corresponding portion of the nucleotide sequence of SEQ ID NO: 14,
b) a sense strand comprising at least 15 contiguous nucleotides differing by no more than 3 nucleotides from the nucleotide sequence of SEQ ID NO: 17 and an antisense strand comprising a nucleotide sequence comprising at least 15 contiguous nucleotides differing by no more than 3 nucleotides from the corresponding portion of the nucleotide sequence of SEQ ID NO: 18,
c) a sense strand comprising at least 15 contiguous nucleotides differing by no more than 3 nucleotides from the nucleotide sequence of SEQ ID NO: 19 and an antisense strand comprising a nucleotide sequence comprising at least 15 contiguous nucleotides differing by no more than 3 nucleotides from the corresponding portion of the nucleotide sequence of SEQ ID NO:20,
d) a dsRNA agent comprising an antisense strand comprising a nucleotide sequence selected from the group consisting of any of the antisense strand nucleotide sequences in any one of Tables 2, 3, 10A, 10C, 11, and 12;
e) a dsRNA agent comprising a sense strand comprising at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13; and
f) a dsRNA agent comprising a sense strand comprising at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and an antisense strand comprising at least 15 contiguous nucleotides from the corresponding nucleotide sequence of SEQ ID NO:14; and
wherein a second dsRNA agent targeting the sense strand of C9orf72 is selected from the group consisting of
a) a sense strand comprising at least 15 contiguous nucleotides differing by no more than 3 nucleotides from the nucleotide sequence of SEQ ID NO: 1 and an antisense strand comprising a nucleotide sequence comprising at least 15 contiguous nucleotides differing by no more than 3 nucleotides from the corresponding portion of the nucleotide sequence of SEQ ID NO:5,
b) a sense strand comprising at least 15 contiguous nucleotides differing by no more than 3 nucleotides from the nucleotide sequence of SEQ ID NO: 15 and an antisense strand comprising a nucleotide sequence comprising at least 15 contiguous nucleotides differing by no more than 3 nucleotides from the corresponding portion of the nucleotide sequence of SEQ ID NO:16,
c) a dsRNA agent comprising an antisense strand comprising at least 15 contiguous nucleotides differing by no more than 3 nucleotides from any one of the antisense nucleotide sequences in any one of Tables 5, 6, 10B, and 10D;
d) a dsRNA agent comprising a sense strand comprising at least 15 contiguous nucleotides differing by no more than three nucleotides from any one of the nucleotide sequence of nucleotides 1-23; 15-37; 33-55; 37-59; 59-81, 62-84, 69-91 of SEQ ID NO: 1, and an antisense strand comprising at least 15 contiguous nucleotides from the corresponding nucleotide sequence of SEQ ID NO:5;
e) a dsRNA agent comprising a sense strand comprising at least 15 contiguous nucleotides differing by no more than three nucleotides from any one of the nucleotide sequence of nucleotides 5197-5219; 5213-5235; 5223-5245, 5226-5248; 5227-5249, 5228-5250, 5229-5251, 5230-5252, 5231-5253, 5233-5255; 5235-5256, 5241-5263; 5245-5267; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and an antisense strand comprising at least 15 contiguous nucleotides from the corresponding nucleotide sequence of SEQ ID NO:16;
f) a dsRNA agent comprising a sense strand comprising at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and an antisense strand comprising at least 15 contiguous nucleotides from the corresponding nucleotide sequence of SEQ ID NO:16;
g) a dsRNA agent comprising a sense strand comprising at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and an antisense strand compriing at least 15 contiguous nucleotides from the corresponding nucleotide sequence of SEQ ID NO:5; and
h) a dsRNA agent comprising an antisense strand comprising at least 15 contiguous nucleotides differing by no more than 3 nucleotides from any one of the antisense nucleotide sequences in any one of Tables 8 and 9; and
wherein the sense strand of the first dsRNA, the antisense strand of the first dsRNA, both the sense strand and the antisense strand of the first dsRNA, the sense strand of the second dsRNA, the antisense strand of the second dsRNA, and/or both the sense strand and the antisense strand of the second dsRNA comprises at least one modified nucleotide.

In one embodiment, the sense strand or the antisense strand is a sense strand or an antisense strand selected from the sense strand or antisense strand of a duplex selected from the group consisting of AD-1446213.1; AD-1446217.1; AD-1446222.1; AD-1446234.1; AD-1446243.1; AD-1446246.1; AD-1446252.1; AD-1446259.1; AD-1446265.1; AD-1446268.1; AD-1446271.1; AD-1446279.1; AD-1446289.1; and AD-1446294.1.

In one embodiment, the sense strand or the antisense strand is a sense strand or an antisense strand selected from the sense strand or antisense strand of a duplex selected from the group consisting of AD-1446213.1; AD-1446246.1; and AD-1446268.1.

In one embodiment, the antisense strand comprises at least 15 contiguous nucleotides differing by no more than three, two or one nucleotides from any one of the antisense strand nucleotide sequences and/or the sense strand nucleotide sequences of a duplex selected from the group consisting of AD-1446073.1; AD-1446075.1; AD-1285246.2; AD-1446084.1; AD-1446087.1; AD-1446090.1, and AD1446095.1.

In one embodiment, the antisense strand comprises at least 15 contiguous nucleotides differing by no more than three, two or one nucleotides from any one of the antisense strand nucleotide sequences and/or the sense strand nucleotide sequences of a duplex selected from the group consisting of AD-1285231.1, AD-1285232.1. AD-1285233.1. AD-1285235.1. AD-1285237.1. AD-1285239.1. AD-1285240.1. AD-1285242.1. AD-1285244.1; AD-1285238.1; AD-1285234.1; AD-1285243.1; AD-1285241.1; AD-1285236.1; AD-1446111.1; AD-1446117.1; AD-1446147.1; AD-1446157.1; AD-1446168.1; AD-1446180.1; AD-1446189.1; AD-1446196.1; AD-1446202.1; AD-1446205.1.

In one embodiment, the antisense strand comprises at least 15 contiguous nucleotides differing by no more than three, two or one nucleotides from any one of the antisense strand nucleotide sequences and/or the sense strand nucleotide sequences of a duplex selected from the group consisting of AD-1285238.1 and AD-1285234.1.

In one embodiment, the antisense strand of the first dsRNA agent comprises at least 15 contiguous nucleotides differing by no more than three, two or one nucleotides from any one of the antisense strand nucleotide sequences and or the sense strand nucleotide sequences of a duplex selected from the group consisting of AD-1446213.1. AD-1446246.1, and AD-1446268.1; and the antisense strand of the second dsRNA agent comprises at least 15 contiguous nucleotides differing by no more than three, two or one nucleotides from any one of the antisense strand nucleotide sequences and/or the sense strand nucleotide sequences of a duplex selected from the group consisting of AD-1285238.1 and AD-1285234.1.

In one embodiment, a) the first dsRNA agent comprises an antisense strand comprising a nucleotide sequence comprising at least 15 contiguous nucleotides differing by no more than 3 nucleotides from the antisense sequence of AD-1446213; and the second dsRNA agent comprises an antisense strand comprising a nucleotide sequence comprising at least 15 contiguous nucleotides differing by no more than 3 nucleotides from the antisense sequence of AD-1285238;
b) the first dsRNA agent comprises an antisense strand comprising a nucleotide sequence comprising at least 15 contiguous nucleotides differing by no more than 3 nucleotides from the antisense sequence of AD-1446213; and the second dsRNA agent comprises an antisense strand comprising a nucleotide sequence comprising at least 15 contiguous nucleotides differing by no more than 3 nucleotides from the antisense sequence of AD-1285234;
c) the first dsRNA agent comprises an antisense strand comprising a nucleotide sequence comprising at least 15 contiguous nucleotides differing by no more than 3 nucleotides from the antisense sequence of AD-1446246; and the second dsRNA agent comprises an antisense strand comprising a nucleotide sequence comprising at least 15 contiguous nucleotides differing by no more than 3 nucleotides from the antisense sequence of AD-1285238;
d) the first dsRNA agent comprises an antisense strand comprising a nucleotide sequence comprising at least 15 contiguous nucleotides differing by no more than 3 nucleotides from the antisense sequence of AD-1446246; and the second dsRNA agent comprises an antisense strand comprising a nucleotide sequence comprising at least 15 contiguous nucleotides differing by no more than 3 nucleotides from the antisense sequence of AD-1285234;
e) the first dsRNA agent comprises an antisense strand comprising a nucleotide sequence comprising at least 15 contiguous nucleotides differing by no more than 3 nucleotides from the antisense sequence of AD-1446268; and the second dsRNA agent comprises an antisense strand comprising a nucleotide sequence comprising at least 15 contiguous nucleotides differing by no more than 3 nucleotides from the antisense sequence of AD-1285238;
f) the first dsRNA agent comprises an antisense strand comprising a nucleotide sequence comprising at least 15 contiguous nucleotides differing by no more than 3 nucleotides from the antisense sequence of AD-1446268; and the second dsRNA agent comprises an antisense strand comprising a nucleotide sequence comprising at least 15 contiguous nucleotides differing by no more than 3 nucleotides from the antisense sequence of AD-1285234.

In another embodiment, a) the first dsRNA agent comprises the antisense strand and/or the sense strand of AD-1446213 and the second dsRNA agent comprises the antisense strand and/or the sense strand of AD-1285238;
b) the first dsRNA agent comprises the antisense strand and/or the sense strand of AD-1446213 and the second dsRNA agent comprises the antisense strand and/or the sense strand of AD-1285234;
c) the first dsRNA agent comprises the antisense strand and/or the sense strand of AD-1446246 and the second dsRNA agent comprises the antisense strand and/or the sense strand of AD-1285238;
d) the first dsRNA agent comprises the antisense strand and/or the sense strand of AD-1446246 and the second dsRNA agent comprises the antisense strand and/or the sense strand of AD-1285234;
e) the first dsRNA agent comprises the antisense strand and/or the sense strand of AD-1446268 and the second dsRNA agent comprises the antisense strand and/or the sense strand of AD-1285238; or
f) the first dsRNA agent comprises the antisense strand and/or the sense strand of AD-1446268 and the second dsRNA agent comprises the antisense strand and/or the sense strand of AD-1285234.

In one embodiment, the sense strand, the antisenses strand, or both the sense strand and the antisense strand is conjugated to one or more lipophilic moieties.

In one embodiment, the lipophilic moiety is conjugated to one or more internal positions in the double stranded region of the first dsRNA agent, the second dsRNA agent or both the first and second dsRNA agent.

In one embodiment, the lipophilic moiety is conjugated to the first dsRNA agent, the second dsRNA agent or both the first and second dsRNA agent via a linker or carrier.

In one embodiment, lipophilicity of the lipophilic moiety, measured by logKow, conjugated to the first dsRNA agent, the second dsRNA agent or both the first and second dsRNA agent exceeds 0.

In one embodiment, the hydrophobicity of the first dsRNA agent, the second dsRNA agent or both the first and the second dsRNA agents, measured by the unbound fraction in a plasma protein binding assay of the dsRNA agent, exceeds 0.2.

In one embodiment, the plasma protein binding assay is an electrophoretic mobility shift assay using human serum albumin protein.

In one embodiment, the first dsRNA agent, the second dsRNA agent or both the first and second dsRNA agents comprise at least one modified nucleotide.

In one embodiment, no more than five of the sense strand nucleotides and no more than five of the antisense strand nucleotides of the first dsRNA agent, the second dsRNA agent or both the first and second dsRNA agent are unmodified nucleotides.

In one embodiment, all of the nucleotides of the sense strand and all of the nucleotides of the antisense strand of the first dsRNA agent, the second dsRNA agent or both the first and second dsRNA agent are modified nucleotides.

In one embodiment, at least one of the modified nucleotides is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a 2′-O-hexadecyl nucleotide, a 2′-phosphate nucleotide, a locked nucleotide, an unlocked nucleotide, a conformationally restricted nucleotide, a constrained ethyl nucleotide, an abasic nucleotide, an inverted abasic residue, a 2′-amino-modified nucleotide, a 2′-O-allyl-modified nucleotide, 2′-C-alkyl-modified nucleotide, 2′-hydroxy-modified nucleotide, a 2′-methoxyethyl modified nucleotide, a 2′-O-alkyl-modified nucleotide, 2′,3′-seco-modified nucleotide, a morpholino nucleotide, a phosphoramidate, a non-natural base comprising nucleotide, a tetrahydropyran modified nucleotide, a 1.5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a glycol modified nucleic acid (GNA), a nucleotide comprising adenosine-glycol nucleic acid (GNA), a nucleotide comprising thymidine-glycol nucleic acid (GNA)S-Isomer, a nucleotide comprising 2-hydroxymethyl-tetrahydrofuran-5-phosphate, a nucleotide comprising 2′-deoxythymidine-3′phosphate, a nucleotide comprising 2′-deoxyguanosine-3′-phosphate, and a terminal nucleotide linked to a cholesteryl derivative and a dodecanoic acid bisdecylamide group; and combinations thereof.

In one embodiment, the modified nucleotide is selected from the group consisting of a 2′-deoxy-2′-fluoro modified nucleotide, a 2′-deoxy-modified nucleotide, 3′-terminal deoxy-thymine nucleotides (dT), a locked nucleotide, 2′-O-hexadecyl nucleotide, a 2′-phosphate nucleotide, a glycol nucleotide, a vinyl-phosphonate nucleotide, an abasic nucleotide, a 2′-amino-modified nucleotide, a 2′-alkyl-modified nucleotide, a morpholino nucleotide, a phosphoramidate, and a non-natural base comprising nucleotide.

In one embodiment, the modified nucleotide comprises a short sequence of 3′-terminal deoxy-thymine nucleotides (dT).

In one embodiment, the modified nucleotides are independently selected from the group consisting of: 2′-O-methyl modified nucleotides, GNA modified nucleotides, 2′-O-hexadecyl modified nucleotides, 2′-phosphate modified nucleotides, vinyl-phosphonate modified nucleotides, and 2′fluoro modified nucleotides.

In one embodiment, the first dsRNA agent, the second dsRNA agent or both the first and second dsRNA agents comprise at least one phosphorothioate internucleotide linkage.

In one embodiment, the first dsRNA agent, the second dsRNA agent or both the first and second dsRNA agents comprise 6-8 phosphorothioate internucleotide linkages.

In one embodiment, each strand of the first dsRNA agent, the second dsRNA agent or both the first and second dsRNA agents is no more than 30 nucleotides in length.

In one embodiment, at least one strand of the first dsRNA agent, the second dsRNA agent or both the first and second dsRNA agents comprises a 3′ overhang of at least 1 nucleotide.

In one embodiment, at least one strand of the first dsRNA agent, the second dsRNA agent or both the first and second dsRNA agents comprise a 3′ overhang of at least 2 nucleotides.

In one embodiment, the double stranded region of the first dsRNA agent, the second dsRNA agent or both the first and second dsRNA agent is 15-30 nucleotide pairs in length.

In one embodiment, the double stranded region of the first dsRNA agent, the second dsRNA agent or both the first and second dsRNA agent is 17-23 nucleotide pairs in length.

In one embodiment, the double stranded region of the first dsRNA agent, the second dsRNA agent or both the first and second dsRNA agents is 17-25 nucleotide pairs in length.

In one embodiment, the double stranded region of the first dsRNA agent, the second dsRNA agent or both the first and second dsRNA agents is 23-27 nucleotide pairs in length.

In one embodiment, the double stranded region of the first dsRNA agent, the second dsRNA agent or both the first and second dsRNA agents is 19-21 nucleotide pairs in length.

In one embodiment, the double stranded region of the first dsRNA agent, the second dsRNA agent or both the first and second dsRNA agents is 21-23 nucleotide pairs in length.

In one embodiment, each strand of the first dsRNA agent, the second dsRNA agent or both the first and second dsRNA agents is 19-30 nucleotides in length.

In one embodiment, each strand of the first dsRNA agent, the second dsRNA agent or both the first and second dsRNA agents is 19-23 nucleotides in length.

In one embodiment, each strand of the first dsRNA agent, the second dsRNA agent or both the first and second dsRNA agents is 21-23 nucleotides in length.

In one embodiment, the one or more lipophilic moieties are conjugated to one or more internal positions on at least one strand of the first dsRNA agent, the second dsRNA agent or both the first and second dsRNA agents via a linker or carrier.

In one embodiment, the internal positions of the first dsRNA agent, the second dsRNA agent or both the first and second dsRNA agents include any positions except the terminal two positions from each end of the at least one strand.

In one embodiment, the internal positions of the first dsRNA agent, the second dsRNA agent or both the first and second dsRNA agents include any positions except the terminal three positions from each end of the at least one strand.

In one embodiment, the internal positions of the first dsRNA agent, the second dsRNA agent or both the first and second dsRNA agents exclude a cleavage site region of the sense strand.

In one embodiment, the internal positions of the first dsRNA agent, the second dsRNA agent or both the first and second dsRNA agents include any positions except positions 9-12, counting from the 5′-end of the sense strand.

In one embodiment, the internal positions of the first dsRNA agent, the second dsRNA agent or both the first and second dsRNA agents include any positions except positions 11-13, counting from the 3′-end of the sense strand.

In one embodiment, the internal positions of the first dsRNA agent, the second dsRNA agent or both the first and second dsRNA agents exclude a cleavage site region of the antisense strand.

In one embodiment, the internal positions of the first dsRNA agent, the second dsRNA agent or both the first and second dsRNA agents include any positions except positions 12-14, counting from the 5′-end of the antisense strand.

In one embodiment, the internal positions of the first dsRNA agent, the second dsRNA agent or both the first and second dsRNA agents include any positions except positions 11-13 on the sense strand, counting from the 3′-end, and positions 12-14 on the antisense strand, counting from the 5′-end.

In one embodiment, the one or more lipophilic moieties are conjugated to one or more of the internal positions of the first dsRNA agent, the second dsRNA agent or both the first and second dsRNA agents selected from the group consisting of positions 4-8 and 13-18 on the sense strand, and positions 6-10 and 15-18 on the antisense strand, counting from the 5′ end of each strand.

In one embodiment, the one or more lipophilic moieties are conjugated to one or more of the internal positions of the first dsRNA agent, the second dsRNA agent or both the first and second dsRNA agents selected from the group consisting of positions 5, 6, 7, 15, and 17 on the sense strand, and positions 15 and 17 on the antisense strand, counting from the 5′-end of each strand.

In one embodiment, the internal positions in the double stranded region of the first dsRNA agent, the second dsRNA agent or both the first and second dsRNA agents exclude a cleavage site region of the sense strand.

In one embodiment, the sense strand is 21 nucleotides in length, the antisense strand is 23 nucleotides in length, and the lipophilic moiety is conjugated to position 21, position 20, position 15, position 1, position 7, position 6, or position 2 of the sense strand or position 16 of the antisense strand, counting from the 5′-end.

In one embodiment, the lipophilic moiety is conjugated to the first dsRNA agent, the second dsRNA agent or both the first and second dsRNA agents at position 21, position 20, position 15, position 1, or position 7 of the sense strand, counting from the 5′-end.

In one embodiment, the lipophilic moiety is conjugated to the first dsRNA agent, the second dsRNA agent or both the first and second dsRNA agents at position 21, position 20, or position 15 of the sense strand, counting from the 5′-end.

In one embodiment, the lipophilic moiety is conjugated to the first dsRNA agent, the second dsRNA agent or both the first and second dsRNA agents at position 20 or position 15 of the sense strand, counting from the 5′-end.

In one embodiment, the lipophilic moiety is conjugated to the first dsRNA agent, the second dsRNA agent or both the first and second dsRNA agents at position 16 of the antisense strand, counting from the 5′-end.

In one embodiment, the lipophilic moiety conjugated to the first dsRNA agent, the second dsRNA agent or both the first and second dsRNA agents is an aliphatic, alicyclic, or polyalicyclic compound.

In one embodiment, the lipophilic moiety is selected from the group consisting of lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, 03-(oleoyl)lithocholic acid, O3-(oleoyl)cholenic acid, dimethoxytrityl, or phenoxazine.

In one embodiment, the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne.

In one embodiment, the lipophilic moiety contains a saturated or unsaturated C6-C18 hydrocarbon chain.

In one embodiment, the lipophilic moiety contains a saturated or unsaturated C16 hydrocarbon chain.

In one embodiment, the saturated or unsaturated C16 hydrocarbon chain is conjugated to position 6, counting from the 5′-end of the strand.

In one embodiment, the lipophilic moiety is conjugated to the first dsRNA agent, the second dsRNA agent or both the first and second dsRNA agents via a carrier that replaces one or more nucleotide(s) in the internal position(s) or the double stranded region.

In one embodiment, the carrier is a cyclic group selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone.

In one embodiment, the lipophilic moiety is conjugated to the double-stranded iRNA agent of the first dsRNA agent, the second dsRNA agent or both the first and second dsRNA agents via a linker containing an ether, thioether, urea, carbonate, amine, amide, maleimide-thioether, disulfide, phosphodiester, sulfonamide linkage, a product of a click reaction, or carbamate.

In one embodiment, the lipophilic moiety is conjugated to a nucleobase, sugar moiety, or internucleosidic linkage of the first dsRNA agent, the second dsRNA agent or both the first and second dsRNA agents.

In one embodiment, the lipophilic moiety or targeting ligand is conjugated to the first dsRNA agent, the second dsRNA agent or both the first and second dsRNA agents via a bio-cleavable linker selected from the group consisting of DNA, RNA, disulfide, amide, functionalized monosaccharides or oligosaccharides of galactosamine, glucosamine, glucose, galactose, mannose, and combinations thereof.

In one embodiment, the 3′ end of the sense strand the first dsRNA agent, the second dsRNA agent or both the first and second dsRNA agents is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the first dsRNA agent, the second dsRNA agent or both the first and second dsRNA agents further comprises a targeting ligand that targets a neuronal cell, a cell in a neuronal tissue, or a cell in a central nervous system tissue, or a liver tissue.

In one embodiment, the targeting ligand is a GalNAc conjugate.

In one embodiment, the first dsRNA agent, the second dsRNA agent or both the first and second dsRNA agents further comprises a terminal, chiral modification occurring at the first internucleotide linkage at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp configuration or Sp configuration.

In one embodiment, the first dsRNA agent, the second dsRNA agent or both the first and second dsRNA agents further comprises a terminal, chiral modification occurring at the first and second internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In one embodiment, the first dsRNA agent, the second dsRNA agent or both the first and second dsRNA agents further comprises a terminal, chiral modification occurring at the first, second and third internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In one embodiment, the first dsRNA agent, the second dsRNA agent or both the first and second dsRNA agents further comprises a terminal, chiral modification occurring at the first, and second internucleotide linkages at the 3′ end of the antisense strand, having the linkage phosphorus atom in Sp configuration, a terminal, chiral modification occurring at the third internucleotide linkages at the 3′ end of the antisense strand, having the linkage phosphorus atom in Rp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In one embodiment, the first dsRNA agent, the second dsRNA agent or both the first and second dsRNA agents further comprises a terminal, chiral modification occurring at the first, and second internucleotide linkages at the 3′ end of the antisense strand, having the linkage phosphorus atom in Sp configuration, a terminal, chiral modification occurring at the first, and second internucleotide linkages at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In one embodiment, the first dsRNA agent, the second dsRNA agent or both the first and second dsRNA agents further comprises a phosphate or phosphate mimic at the 5′-end of the antisense strand.

In one embodiment, the phosphate mimic is a 5′-vinyl phosphonate (VP).

In one embodiment, the base pair at the 1 position of the 5′-end of the antisense strand of the duplex of the first dsRNA agent, the second dsRNA agent or both the first and second dsRNA agents is an AU base pair.

In one embodiment, the sense strand is 21 nucleotides in length and the antisense strand is 23 nucleotides in length.

The present invention also provides cells comprising a composition of the invention.

In some embodiments, the compositions of the invention are pharmaceutical compositions and, in some embodiments, comprise a lipid formulation.

In one aspect, the present invention provides a method of reducing the level of one or more C9orf72 RNA transcripts, such as a C9orf72 RNA containing a hexanucleotide-repeat, such as a C9orf72 gene comprising multiple contiguous copies of a hexanucleotide repeat, in a cell, e.g., a neuron, such as a motor neuron, the method comprising contacting the cell with a dsRNA agent of the invention, two or more, e.g., 2, 3, or 4, dsRNA agents of the invention, a composition comprising two or more, e.g., 2, 3, or 4, dsRNA agents for inhibiting expression of one or more C9orf72 RNA transcripts, e.g., a first dsRNA agent targeting a C9orf72 sense transcript (an exon or intron of C9orf72) and a second dsRNA agent targeting an C9orf72 antisense transcript (an exon or intron of C9orf72) as described herein, or a pharmaceutical composition of the invention, thereby inhibiting expression of the C9orf72 gene in the cell.

In another aspect, the present invention provides methods of reducing dipeptide repeat protein synthesis or dipeptide repeat protein aggregates in a cell. The methods include introducing into the cell a dsRNA agent of the invention, two or more, e.g., 2, 3, or 4, dsRNA agents of the invention, a composition comprising two or more, e.g., 2, 3, or 4, dsRNA agents for inhibiting expression of one or more C9orf72 RNA transcripts, e.g., a first dsRNA agent targeting a C9orf72 sense transcript (an exon or intron of C9orf72) and a second dsRNA agent targeting a C9orf72 antisense transcript (an exon or intron of C9orf72) as described herein, or a pharmaceutical composition of the invention, thereby reducing dipeptide repeat protein synthesis or dipeptide repeat protein aggregates in the cell.

In another aspect, the present invention provides methods of reducing accumulation or aggregation of poly(glycine-alanine) peptides, poly(glycine-proline) peptides, poly(glycine-arginine) peptides, poly(alanine-proline) peptides, or poly(proline-arginine) peptides in a cell. The methods include introducing into the cell a dsRNA agent of the invention, two or more, e.g., 2, 3, or 4, dsRNA agents of the invention, a composition comprising two or more, e.g., 2, 3, or 4, dsRNA agents for inhibiting expression of C9orf72, e.g., a first dsRNA agent targeting a C9orf72 sense transcript (an exon or intron of C9orf72) and a second dsRNA agent targeting a C9orf72 antisense transcript (an exon or intron of C9orf72) as described herein, or a pharmaceutical composition of the invention, thereby reducing accumulation or aggregation of poly(glycine-alanine) peptides, poly(glycine-proline) peptides, poly(glycine-arginine) peptides, poly(alanine-proline) peptides, or poly(proline-arginine) peptides in the cell.

In another aspect, the present invention provides methods of reducing repeat-length-dependent formation of C9orf72 RNA foci in a cell. The methods include introducing into the cell a dsRNA agent of the invention, two or more, e.g., 2, 3, or 4, dsRNA agents of the invention, a composition comprising two or more, e.g., 2, 3, or 4, dsRNA agents for inhibiting expression of C9orf72, e.g., a first dsRNA agent targeting a C9orf72 sense transcript (an exon or intron of C9orf72) and a second dsRNA agent targeting a C9orf72 antisense transcript (an exon or intron of C9orf72) as described herein, or a pharmaceutical composition of the invention, thereby reducing repeat-length-dependent formation of C9orf72 RNA foci in the cell.

In another aspect, the present invention provides methods of reducing nuclear and/or cytoplasmic sense and/or antisense C9orf72 RNA foci in a cell. The methods include introducing into the cell a dsRNA agent of the invention, two or more, e.g., 2, 3, or 4, dsRNA agents of the invention, a composition comprising two or more, e.g., 2, 3, or 4, dsRNA agents for inhibiting expression of C9orf72, e.g., a first dsRNA agent targeting a C9orf72 sense transcript (an exon or intron of C9orf72) and a second dsRNA agent targeting a C9orf72 antisense transcript (an exon or intron of C9orf72) as described herein, or a pharmaceutical composition of the invention, thereby reducing nuclear and/or cytoplasmic sense and/or antisense C9orf72 RNA foci in the cell.

In one embodiment, cell is within a subject.

In one embodiment, the subject is a human.

In one embodiment, the subject has or is at risk of developing a C9orf72-associated disorder, such as a C9orf72-hexanucleotide-repeat-expansion-associated disease, condition, or disorder.

In one embodiment, the C9orf72-associated disorder is selected from the group consisting of C9orf72 amyotrophic lateral sclerosis, frontotemporal dementia, Hungtinton's disease Huntington-Like Syndrome Due To C9orf72 hexanucletoide repeat expansions, parkinsonism, olivopontocerebellar degeneration, corticobasal syndrome, and Alzheimer's disease.

In one embodiment, ontacting the cell with the dsRNA agent inhibits the levels of sense and/or antisense hexanucleotide-repeat-containing C9orf72 RNA transcripts by at least 50%, 60%, 70%, 80%, 90%, or 95%.

In one embodiment, inhibiting the levels of sense and/or antisense hexanucleotide-repeat-containing C9orf72 RNA transcripts decreases the level of one or more aberrant dipeptide-repeat (DPR) proteins selected from the group consisting of poly(glycine-alanine), poly(glycine-arginine), poly(glycine-proline), poly(proline-alanine), and poly(proline-arginine) by at least 50%, 60%, 70%, 80%, 90%, or 95%.

In one embodiment, contacting the cell with the dsRNA agent inhibits the expression of C9orf72 mRNA by no more than 50%, 40%, 30%, 20%, 10% or 5%.

In one embodiment, the dsRNA agent inhibits expression of a C9orf72 target mRNA comprising the hexanucleotide repeat by at least 10%, at least 20%, at least 30%, at least 40%, at least 50%, or at least 60% within 24-48 hours after administration to a cell expressing the C9orf72 target RNA comprising the hexanucleotide repeat.

In some embodiments, the dsRNA agent selectively inhibits expression of a C9orf72 target RNA comprising the hexanucleotide repeat relative to expression of a mature C9orf72 messenger RNA. In other embodiments, the dsRNA agent inhibits expression of a mature C9orf72 messenger RNA by less than 50%, less than 40%, less than 30%, less than 20%, or less than 10% within 24-48 hours after administration to a cell expressing the mature C9orf72 messenger RNA.

In some embodiments, the dsRNA agent reduces dipeptide repeat (poly(GA), poly(GR), poly(GP), poly(PA), and/or poly(PR)) protein synthesis or dipeptide repeat (poly(GA), poly(GR), poly(GP), poly(PA), and/or poly(PR)) protein aggregates in the cell.

In some embodiments, the dsRNA agent reduces nuclear and/or cytoplasmic sense and/or antisense C9orf72 RNA foci in the cell.

In one embodiment, inhibiting expression of C9orf72 decreases C9orf72 protein level in serum of the subject by no more than 50%, 40%, 30%, 20%, 10% or 5%.

In some embodiments, the dsRNA agent reduces dipeptide repeat (poly(GA), poly(GR), poly(GP), poly(PA), and/or poly(PR)) protein synthesis or dipeptide repeat (poly(GA), poly(GR), poly(GP), poly(PA), and/or poly(PR)) protein aggregates by at least 10%, at least 20%, at least 30%, at least 40%, at least 50%, at least 60%, at least 70%, or at least 80% within 24-48 hours after administration to the cell.

In one aspect, the present invention provides methods of treating a subject having a disorder that would benefit from knocking down a target C9orf72 RNA, such as a C9orf72-hexanucleotide-repeat-expansion-associated disease, condition, or disorder, comprising administering to the subject a therapeutically effective amount of a dsRNA agent of the invention, two or more, e.g., 2, 3, or 4, dsRNA agents of the invention, a composition comprising two or more, e.g., 2, 3, or 4, dsRNA agents for inhibiting expression of one or more C9orf72 RNAs, e.g., a first dsRNA agent targeting a C9orf72 sense strand transcript (an exon or intron of C9orf72) and a second dsRNA agent targeting a C9orf72 antisense strand transcript (an exon or intron of C9orf72) as described herein, or a pharmaceutical composition of the invention, thereby treating the subject having the disorder that would benefit from reduction in C9orf72 expression.

In another aspect, the present invention provides a method of preventing at least one symptom in a subject having a disorder that would benefit from reduction in expression of a C9orf72 RNA containing a hexanucleotide repeat expansion, such as a C9orf72-hexanucleotide-repeat-expansion-associated disease, condition, or disorder, comprising administering to the subject a prophylactically effective amount of a dsRNA agent of the invention, two or more, e.g., 2, 3, or 4, dsRNA agents of the invention, a composition comprising two or more, e.g., 2, 3, or 4, dsRNA agents for inhibiting expression of C9orf72, e.g., a first dsRNA agent targeting a C9orf72 sense strand transcript (an exon or intron of C9orf72) and a second dsRNA agent targeting a C9orf72 antisense strand transcript (an exon or intron of C9orf72) as described herein, or a pharmaceutical composition of the invention, thereby preventing at least one symptom in the subject having the disorder that would benefit from reduction in C9orf72 expression.

In one embodiment, the methods include administering a first dsRNA agent targeting a sense strand of C9orf72 (an exon or intron of C9orf72) and a second dsRNA agent targeting an antisense strand of C9orf72 (an exon or intron of C9orf72).

In some embodiments, suitable agents targeting a sense strand of C9orf72 for use in the methods of the invention comprising two or more dsRNA agents comprise a sense strand and an antisense strand forming a double stranded region selected from the group consisting of
a) a sense strand comprising at least 15 contiguous nucleotides differing by no more than 3 nucleotides from the nucleotide sequence of SEQ ID NO:1 and an antisense strand comprising a nucleotide sequence comprising at least 15 contiguous nucleotides differing by no more than 3 nucleotides from the corresponding portion of the nucleotide sequence of SEQ ID NO:5,
b) a sense strand comprising at least 15 contiguous nucleotides differing by no more than 3 nucleotides from the nucleotide sequence of SEQ ID NO: 15 and an antisense strand comprising a nucleotide sequence comprising at least 15 contiguous nucleotides differing by no more than 3 nucleotides from the corresponding portion of the nucleotide sequence of SEQ ID NO: 16,
c) an antisense strand comprising at least 15 contiguous nucleotides differing by no more than 3 nucleotides from any one of the antisense nucleotide sequences in any one of Tables 5, 6, 10B, and 10D;
d) a sense strand comprising at least 15 contiguous nucleotides differing by no more than three nucleotides from any one of the nucleotide sequence of nucleotides 1-23; 15-37; 33-55; 37-59; 59-81; 62-84, or 69-91 of SEQ ID NO: 1, and an antisense strand comprising at least 15 contiguous nucleotides from the corresponding nucleotide sequence of SEQ ID NO:5;
e) a sense strand comprising at least 15 contiguous nucleotides differing by no more than three nucleotides from any one of the nucleotide sequence of nucleotides 5197-5219; 5213-5235; 5223-5245; 5226-5248; 5227-5249; 5228-5250, 5229-5251, 5230-5252, 5231-5253, 5235-5256, 5241-5263; 5245-5267; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and an antisense strand comprising at least 15 contiguous nucleotides from the corresponding nucleotide sequence of SEQ ID NO:16;
f) a sense strand comprising at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and an antisense strand comprising at least 15 contiguous nucleotides from the corresponding nucleotide sequence of SEQ ID NO:16;
g) a sense strand comprising at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and an antisense strand comprising at least 15 contiguous nucleotides from the corresponding nucleotide sequence of SEQ ID NO:5; and
h) an antisense strand comprising at least 15 contiguous nucleotides differing by no more than 3 nucleotides from any one of the antisense nucleotide sequences in any one of Tables 8 and 9, wherein the sense strand, the antisense strand, or both the sense strand and the antisense strand comprises at least one modified nucleotide.

In certain embodiments, suitable agents targeting a sense strand of C9orf72, e.g, of a C9orf72 exon or intron sense sequence, for use in the methods of the invention comprising two or more dsRNA agents are those dsRNA agents disclosed in PCT Publication No. WO 2021/119226, the entire contents of which are incorporated herein by reference.

In certain embodiments, suitable agents targeting an antisense strand of C9orf72 for use in the methods of the invention comprising two or more dsRNA agents comprise a sense strand an an antisense strand forming a double stranded region selected from the group consisting of
a) a sense strand comprising at least 15 contiguous nucleotides differing by no more than 3 nucleotides from the nucleotide sequence of SEQ ID NO: 13 and an antisense strand comprising a nucleotide sequence comprising at least 15 contiguous nucleotides differing by no more than 3 nucleotides from the corresponding portion of the nucleotide sequence of SEQ ID NO: 14,
b) a sense strand comprising at least 15 contiguous nucleotides differing by no more than 3 nucleotides from the nucleotide sequence of SEQ ID NO: 17, and an antisense strand comprising a nucleotide sequence comprising at least 15 contiguous nucleotides differing by no more than 3 nucleotides from the corresponding portion of the nucleotide sequence of SEQ ID NO:18,
c) a sense strand comprising at least 15 contiguous nucleotides differing by no more than 3 nucleotides from the nucleotide sequence of SEQ ID NO: 19, and an antisense strand comprising a nucleotide sequence comprising at least 15 contiguous nucleotides differing by no more than 3 nucleotides from the corresponding portion of the nucleotide sequence of SEQ ID NO:20,
d) an antisense comprising a nucleotide sequence selected from the group consisting of any of the antisense strand nucleotide sequences in any one of Tables 2, 3, 10C, 10B, 11, and 12; and
e) a sense strand comprising at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13;
f) a sense strand comprising at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and an antisense strand comprising at least 15 contiguous nucleotides from the corresponding nucleotide sequence of SEQ ID NO: 14,
wherein the sense strand, the antisense strand, or both the sense strand and the antisense strand comprises at least one modified nucleotide.

In one embodiment, the sense strand, the antisense strand, or both the sense strand and the antisense strand is conjugated to one or more lipophilic moieties.

In one embodiment, the disorder is a C9orf72-associated disorder.

In one embodiment, the C9orf723-associated disorder is selected from the group consisting of C9orf72 amyotrophic lateral sclerosis, frontotemporal dementia, Huntington's disease Huntington-Like Syndrome Due To C9orf72 Expansions, parkinsonism, olivopontocerebellar degeneration, corticobasal syndrome, and Alzheimer's disease.

In one embodiment, the subject is human.

In one embodiment, the administration of the agent to the subject causes a decrease in C9orf72 protein accumulation.

In some embodiments, the method reduces dipeptide repeat protein synthesis or reduces dipeptide repeat protein aggregates in the subject. In some embodiments, the method decreases expression of a C9orf72 target RNA comprising a hexanucleotide repeat comprising multiple contiguous copies of SEQ ID NO: 1 in the subject.

In one embodiment, administration of the agent to the subject causes a decrease in the level of one or more dipeptide-repeat (DPR) proteins selected from the group consisting of poly(glycine-alanine), poly(glycine-arginine), poly(glycine-proline), poly(proline-alanine), and poly(proline-arginine).

In one embodiment, the level of one or more aberrant dipeptide-repeat (DPR) proteins is decreased by more than 50%, 60%, 70%, 80%, 90%, or 95%.

In one embodiment, the level of poly(glycine-alanine) and/or poly(glycine-proline) is decreased by more than 50%, 60%, 70%, 80%, 90%, or 95%.

In one embodiment, the dsRNA agent is administered to the subject at a dose of about 0.01 mg/kg to about 50 mg/kg.

In one embodiment, the dsRNA agent is administered to the subject subcutaneously.

In another embodiment, the dsRNA agent is administered to the subject intrathecally.
In yet another embodiment, the dsRNA agent is administered to the subject intracerebroventricularly.

In one embodiment, the methods of the invention further comprise determining the level of C9orf72 in a sample(s) from the subject.

In one embodiment, the level of C9orf72 in the subject sample(s) is a C9orf72 protein level in a blood, serum, or cerebrospinal fluid sample(s).

In one embodiment, the methods of the invention further comprise administering to the subject an additional therapeutic agent.

In one aspect, the present invention provides a kit comprising any one or more of the dsRNA agents of the invention, a composition of the invention, or a pharmaceutical composition of the invention.

In another aspect, the present invention provides a vial comprising any one or more of the dsRNA agents of the invention, a compostion of the invention, or a pharmaceutical composition of the invention.

In yet another aspect, the present invention provides a syringe comprising any one or more of the dsRNA agents of the invention, a composition of the invention, or a pharmaceutical composition of the invention.

In one embodiment, the RNAi agent is a pharmaceutically acceptable salt thereof. “Pharmaceutically acceptable salts” of each of RNAi agents herein include, but are not limited to, a sodium salt, a calcium salt, a lithium salt, a potassium salt, an ammonium salt, a magnesium salt, an mixtures thereof. One skilled in the art will appreciate that the RNAi agent, when provided as a polycationic salt having one cation per free acid group of the optionally modified phosophodiester backbone and/or any other acidic modifications (e.g., 5′-terminal phosphonate groups). For example, an oligonucleotide of “n” nucleotides in length contains n-1 optionally modified phosophodiesters, so that an oligonucleotide of 21 nt in length may be provided as a salt having up to 20 cations (e.g., 20 sodium cations). Similarly, an RNAi agents having a sense strand of 21 nt in length and an antisense strand of 23 nt in length may be provided as a salt having up to 42 cations (e.g., 42 sodium cations). In the preceding example, where the RNAi agent also includes a 5′-terminal phosphate or a 5′-terminal vinylphosphonate group, the RNAi agent may be provided as a salt having up to 44 cations (e.g., 44 sodium cations).

### BRIEF DESCRIPTION OF THE FIGURES

FIG. 1 is a graph showing the results of a single dose screen in Cos-7 cells of the indicated agents at 10 nM. 1 nM, or 0.1 nM final concentration.

FIG. 2 is a graph showing the results of a subset of the agents from FIG. 1 selected for further analysis based on the single dose screen in Cos-7 cells at 10 nM, 1 nM, or 0.1 nM final concentration.

FIG. 3 is a graph showing the results of a single dose screen in Cos-7 cells of the indicated agents at 10 nM. 1 nM, or 0.1 nM final concentration.

FIG. 4 is a graph showing the results of a subset of the agents from FIG. 3 selected for further analysis based on the single dose screen in Cos-7 cells at 10 nM, 1 nM, or 0.1 nM final concentration.

FIGS. 5A-5B are graphs depicting the effect of duplexes of interest on the accumulation of C9orf72 RNA. Embryonic stem cells carrying an approximately 300X G4C2 repeat expansion were electroporated with 1 μM of two different dsRNA agents targeting sense RNA (solid dark bars) or two different dsRNA agents targeting antisense RNA (white bars) transcribed from the region of the C9orf72 gene between exon 1A and the repeat expansion, or a combination of the sense RNA targeting siRNA-1 (AD1285238.1) and one of each antisense targeting siRNA (hatched bars). Knockdown of transcripts that contain sequences derived from the region of the C9orf72 gene between exon 1A and the repeat expansion (FIG. 5A) was assayed by RT-qPCR with an assay that detects sequence from this region. Note that this assay detects predominantly sense RNA because the antisense RNA level is one-eighth that of the sense RNA. C9orf72 spliced mRNA (FIG. 5B) was assayed by RT-qPCR with an assay that recognizes RNAs that contain sequences that span the junction of exons 2 and 3. Data were normalized to the average of two control samples (black bars) treated with the vehicle, artificial cerebral spinal fluid (aCSF).

FIG. 6A-6C are western slot blots (FIG. 6A) and graphs of the quantification of the blots (FIGS. 6B and 6C) depicting the effect of duplexes of interest on the levels of dipeptide repeat proteins. Embryonic stem cells carrying an approximately 300X G4C2 repeat expansion were electroporated with 1 μM of two different dsRNA agents targeting the sense RNA (solid dark bars, FIGS. 6B-6C), antisense RNA (white bars, FIGS. 6B-6C), or in combination as in FIG. 5 (hatched bars, FIGS. 6B-6C). Levels of dipeptide repeat proteins following knockdown were assayed with antibodies against poly(GlyAla) (right panel FIG. 6A) and poly(GlyPro) (left panel FIG. 6A). Relative proteins levels for poly(GlyPro) (FIG. 6B) and poly(GlyAla) (FIG. 6C) following siRNA treatment were quantitated and normalized to samples treated with aCSF. FIG. 6A discloses SEQ ID NO: 100.

FIG. 7 is a graph depicting the percent C9orf72 mRNA remaining following intrathecal administration of a single 3 mg/kg dose of the indicated duplexes or PBS.

FIG. 8 is a graph depicting the use of Nanostring probes for mapping of the transcription start site in C9orf72 antisense RNA. FIG. 8 discloses SEQ ID NO: 100.

### DETAILED DESCRIPTION OF THE INVENTION

The present disclosure provides RNAi compositions, which affect the RNA-induced silencing complex (RISC)-mediated cleavage of RNA transcripts of a C9orf72 gene, such as a C9orf72 gene having an expanded GGGGCC (G₄C₂) repeat (SEQ ID NO: 100). The C9orf72 gene may be within a cell. e.g., a cell within a subject, such as a human. The use of these iRNAs enables the targeted degradation of RNAs of the corresponding gene (C9orf72 gene) in mammals.

The iRNAs of the invention have been designed to target a C9orf72 target RNA. e.g., a C9orf72 target RNA having an expanded GGGGCC (SEQ ID NO: 100), hexanucleotide repeat in an intron of the gene. The agents may target a mature C9orf72 mRNA (an mRNA having the introns spliced out) or a C9orf7 mRNA precursor (an mRNA containing introns). In certain aspects of the invention, the RNAi agents of the disclosure may target a C9orf72 sense and/or antisense RNA transcript containing a hexanucleotide-repeat (an RNA containing C9orf72 intron 1A). Targeting a C9orf72 sense and/or antisense strand RNA containing a hexanucleotide-repeat can inhibit expression of or reduce the presence of aberrant dipeptide-repeat (DPR) proteins (poly(GA), poly(GR), poly(GP), poly(PA), and poly(PR)), which are produced from all reading frames of either sense or antisense repeat-containing C9orf72 RNAs through repeat-associated non-AUG-dependent (RAN) translation, in cells of the nervous systems of subjects having a C9orf72-associated disease. In some embodiments, a combination of an RNA agent targeting a C9orf72 sense strand RNA containing a hexanucleotide-repeat and an RNA agent targeting a C9orf72 antisense strand RNA containing a hexanucleotide-repeat are provided together.

The described iRNAs may have one or more nucleotide modifications or combination of nucleotide modifications that increase activity, delivery, and/or stability of the iRNAs.

In some embodiments, the iRNAs of the invention inhibit the expression of the C9orf72 gene (e.g., mature mRNA) by no more than about 50%, and reduce the level of sense- and antisense-containing C9orf72 RNA foci, reduce the level of one or more aberrant dipeptide-repeat (DPR) proteins (poly(GA), poly(GR), poly(GP), poly(PA), and poly(PR)), and/or decrease the expression of the C9orf72 sense and/or antisense RNA containing a hexanucleotide-repeat by more than about 50%. Without intending to be limited by theory, it is believed that a combination or sub-combination of the foregoing properties and the specific target sites, or the specific modifications in these iRNAs confer to the iRNAs of the invention improved efficacy, stability, potency, durability, and safety.

Accordingly, the present disclosure also provides methods of using the RNAi compositions of the disclosure, including, compositions comprising one or more, e.g., 2, 3, or 4, dsRNA agents of the invention, for knocking down or inhibiting the expression of one or more C9orf72 RNAs or for treating a subject having a disorder that would benefit from knocking down or inhibiting the expression of one or more C9orf72 RNAs, e.g., a C9orf72-associated disease, for example, a disease associated with an expanded GGGGCC hexanucleotide repeat (SEQ ID NO: 100) in an intron of the C9orf72 gene, such as C9orf72 amyotrophic lateral sclerosis, frontotemporal dementia, or Huntington's disease, e.g., Huntington-Like Syndrome Due To C9orf72 Expansions, parkinsonism, olivopontocerebellar degeneration, corticobasal syndrome, or Alzheimer's disease.

The RNAi agents of the disclosure include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, which region is substantially complementary to at least part of an target RNA transcript of a C9orf72 gene, e.g., a C9orf72 intron. In certain embodiments, the RNAi agents of the disclosure include an RNA strand (the antisense strand) having a region which is about 21-23 nucleotides in length, which region is substantially complementary to at least part of an target RNA transcript of a C9orf72 gene, e.g., a C9orf72 intron.

As the presence of sense and antisense C9orf72-containing foci as well as the presence of aberrant dipeptide-repeat (DPR) proteins (poly(GA), poly(GR), poly(GP), poly(PA), and poly(PR)) produced from all reading frames of either sense or antisense repeat-containing C9orf72 RNAs through repeat-associated non-AUG-dependent (RAN) translation have been identified in several cell types in the nervous systems of subjects having a C9orf72-associated disease (Lagier-Tourenne, et al. (2013) Proc Natl Acad Sci USA doi/10.1073/pnas.1318835110; Jiang, et al. (2016), in certain aspects of the invention, the RNAi agents of the disclosure include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, which region is substantially complementary to at least part of a target RNA transcript of a C9orf72 gene, e.g., a C9orf72 intron. In certain embodiments, the RNAi agents of the disclosure include an RNA strand (the antisense strand) having a region which is about 21-23 nucleotides in length, which region is substantially complementary to at least part of an target RNA transcript of a C9orf72 gene, e.g., a C9orf72 intron.

In certain embodiments, the RNAi agents of the disclosure include an RNA strand (the antisense strand) which can include longer lengths, for example up to 66 nucleotides, e.g., 36-66, 26-36, 25-36, 31-60, 22-43, 27-53 nucleotides in length with a region of at least 19 contiguous nucleotides that is substantially complementary to at least a part of an mRNA transcript of a C9orf72 gene. These RNAi agents with the longer length antisense strands preferably include a second RNA strand (the sense strand) of 20-60 nucleotides in length wherein the sense and antisense strands form a duplex of 18-30 contiguous nucleotides.

The use of these RNAi agents enables the targeted degradation of target RNAs of a C9orf72 gene in mammals. Thus, methods and compositions including these RNAi agents are useful for treating a subject who would benefit by knockdown of a target C9orf72 RNA, a reduction in normal C9orf72 protein and/or or a reduction of the pathogenic dipeptide repeat proteins that are generated from the pathogenic hexnucleotide repeat expansion, such as a subject having a C9orf72-associated disease, such as C9orf72 amyotrophic lateral sclerosis, frontotemporal dementia, Huntington's disease, e.g., Huntington-Like Syndrome Due To C9orf72 Expansions, parkinsonism, olivopontocerebellar degeneration, corticobasal syndrome, or Alzheimer's disease.

The following detailed description discloses how to make and use compositions containing RNAi agents to inhibit the expression of a C9orf72 gene, as well as compositions and methods for treating subjects having diseases and disorders that would benefit from inhibition or reduction of the expression of the genes.

### I. Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a chemical structure and a chemical name, the chemical structure takes precedence.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

The term “C9orf72” gene, also known as “C9orf72-SMCR8 Complex Subunit,” Guaninc Nucleotide Exchange C9orf72.” “Chromosome 9 Open Reading Frame 72, “Protein C9orf72,” “DENNL72,” “FTDALS1,” “ALSFTD”, and “FTDALS,” refers to the gene encoding the well-known protein involved in the regulation of endosomal trafficking, C9orf72. The C9orf72 protein has been shown to interact with Rab proteins that are involved in autophagy and endocytic transport. Expansion of a GGGGCC repeat (SEQ ID NO: 100) from about 2 to about 22 copies to about 700 to about 1600 copies in the intronic sequence between alternate 5′ exons in transcripts from this gene is associated with C9orf72 amyotrophic lateral sclerosis, frontotemporal dementia, Huntington's disease, e.g., Huntington-Like Syndrome Due To C9orf72 Expansions, parkinsonism, olivopontocerebellar degeneration, corticobasal syndrome, or Alzheimer's disease. Alternative splicing results in multiple transcript variants encoding different isoforms.

Exemplary nucleotide and amino acid sequences of C9orf72 can be found, for example, at GenBank Accession No. NM_001256054.2 (Homo sapiens C9orf72, SEQ ID NO: 1, reverse complement SEQ ID NO:5; GenBank Accession No.: XM_005581570.2 (Macaca fascicularis C9orf72, SEQ ID NO:2, reverse complement SEQ ID NO:6); GenBank Accession No. NM_001081343.2 (Mus musculus C9orf72, SEQ ID NO:3, reverse complement SEQ ID NO:7); and GenBank Accession No.: NM_001007702.1 (Rattus norvegicus C9orf72, SEQ ID NO:4, reverse complement SEQ ID NO:8).

Additional nucleotide and amino acid sequences of human C9orf72 can be found, for example, at GenBank Accession No. NM_145005.6, transcript variant 1 (SEQ ID NO:9, reverse complement SEQ ID NO: 10); and NM_018325.5, transcript variant 2 (SEQ ID NO:11, reverse complement SEQ ID NO: 12).

The nucleotide sequence of the genomic region of human chromosome 9 harboring the C9orf72 gene may be found in, for example, the Genome Reference Consortium Human Build 38 (also referred to as Human Genome build 38 or GRCh38) available at GenBank. The nucleotide sequence of the genomic region of human chromosome 9 harboring the C9orf72 gene may also be found at, for example, GenBank Accession No. NC_000009.12 (SEQ ID NO: 13 provides nucleotides 27546546, 27573866 of the assembly of chromosome 9, reverse complement SEQ ID NO:14),. The nucleotide sequence of the human C9orf72 gene may be found in, for example, GenBank Accession No. NG_031977.1 (SEQ ID NO:15, reverese complement, SEQ ID NO:16).

SEQ ID NO: 13 provides nucleotides 27546546, 27573866 of the assembly of chromosome 9 (NC_000009.12). It will be understood when a range for a target sequence within SEQ ID NO: 13 is provided, the nucleotide position range corresponds the nucleotide positions of the assembly of chromosome 9, e.g., nucleotides 27573086-27573106 of SEQ ID NO: 13 refers to the nucleotide positions within the assembly of human chromosome 9, for which SEQ ID NO: 13 provides nucleotides at positions 27546546, 27573866.

Further examples of C9orf72 sequences can be found in publicly available databases, for example, GenBank, OMIM, and UniProt.

Additional information on C9orf72 can be found, for example, at www.ncbi.nlm.nih.gov/gene/203228. The term C9orf72 as used herein also refers to variations of the C9orf72 gene including variants provided in the clinical variant database, for example, at www.ncbi.nlm.nih.gov/clinvar/?term=NM_001256054.2.

The entire contents of each of the foregoing GenBank Accession numbers and the Gene database numbers are incorporated herein by reference as of the date of filing this application.

As used herein, “target sequence” refers to a contiguous portion of the nucleotide sequence of an RNA molecule formed during the transcription of a C9orf72 gene, such as a sense or antisense C9orf72 RNA molecule, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of a C9orf72 gene. In one embodiment, the target sequence is within the protein coding region of a C9orf72 gene. In another embodiment, the target sequence is within an intron, e.g., the intron between exons 1A and 1B, of a C9orf72 gene. In one embodiment, the target sequence is a sense C9orf72 RNA molecule. In another embodiment, the target sequence is an antisense C9orf72 RNA molecule. In one embodiment, the target sequence comprises a transcription start site, e.g., a transcription start site for an antisense C9orf72 RNA molecule, e.g., about 171 bp downstream of the 3′ end of the exon 1B coding DNA, or approximately 270 bp downstream of the GGGGCC hexanucleotide repeat expansion (SEQ ID NO: 100), e.g., nucleotide 5607 of NG_031977 (SEQ ID NO:15). In some embodiments, the target sequence comprises a region between the transcription start site and exon 1A, e.g., nucleotides 5001-5607, 5026-5607, 5127-5607, or 5130-5607 of NG_031977 (SEQ ID NO: 15). Exons 1A and 1B correspond to positions 5001-5158 and 5386-5436 of NG_031977. In some embodiments, the target sequence comprises a region starting from the transcription start site, extending through the hexanucleotide repeat expansion region, and at least about 200 bp, about 500 bp, about 900 bp, about 1200 bp, or about 1500 bp, or about 2000 bp out into the 5′ flanking sequence of the C9orf72 gene. It is understood that if the nucleotide sequence of a target sequence is provided as, e.g., a cDNA or genomic sequence or the reverse complement of a cDNA or genomic sequence, e.g., SEQ ID NOs: 1-20, the “Ts” are “Us” in the corresponding mRNA sequence.

A C9orf72 mRNA (target C9orf72 RNA) is an RNA transcribed from a C9orf72 gene, either a sense strand or an antisense strand transcribed message. A C9orf72 RNA includes C9orf72 mature mRNA, a C9orf72 precursor RNA, or any portions thereof (e.g., spliced out intronic regions or alternatively spliced RNAs). C9orf72 mature mRNA is C9orf72 mRNA in which the introns have been removed (spliced out) and from which C9orf72 protein is translated. C9orf72 precursor RNA is C9orf72 RNA in which at least 1 intron, particularly the first intron (intron 1), has not been removed.

A C9orf72 protein includes any protein expressed from a C9orf72 RNA. A C9orf72 protein includes the protein expressed from C9orf72 mature RNA, as well as dipeptide repeat proteins (e.g., poly(glycine-alanine), poly(glycine-proline), poly(glycine-arginine), poly(alanine-proline), and poly(proline-arginine)) resulting from repeat-associated non-AUG (AUG) translation from C9orf72 RNAs containing hexanucleotide repeats.

A C9orf72 target RNA may include C9orf72 RNA having a hexanucleotide repeat expansion. The hexanucleotide repeat expansion includes, but is not limited to, multiple contiguous copies of SEQ ID NO: 1 or a sequence having at least 90% identity to multiple contiguous copies of SEQ ID NO: 1. The C9orf72 target RNA includes, but is not limited to, C9orf72 sense and antisense RNA transcripts having a hexanucleotide repeat expansion. The C9orf72 target RNA can be, for example, one with a pathogenic hexanucleotide repeat expansion (having, for example, at least about 30, at least about 35, at least about 40, at least about 50, at least about 60, at least about 70, at least about 80, at least about 100, at least about 200, at least about 300, at least about 400, or at least about 500 copies of the hexanucleotide repeat).

The target sequence may be about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G.” “C.” “A.” “T”, and “U” each generally stand for a nucleotide that contains guanine, cytosine, adenine, thymidine, and uracil as a base, respectively in the context of a modified or unmodified nucleotide. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, thymidine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

The terms “IRNA”, “RNAi agent.” “iRNA agent.” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi knocks down (i.e., reduces the amount of) or modulates (i.e., inhibits) the expression of C9orf72, a C9orf72-related transcript, or a C9orf72-related peptide (e.g., a dipeptide repeat) in a cell, e.g., a cell within a subject, such as a mammalian subject.

In one embodiment, an RNAi agent of the disclosure includes a single stranded RNAi that interacts with a target RNA sequence, e.g., a C9orf72 target mRNA sequence (either a sense or an antisense RNA transcript sequence), to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes these dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (ssRNA) (the antisense strand of a siRNA duplex) generated within a cell and which promotes the formation of a RISC complex to effect silencing of the target gene, i.e., a C9orf72 gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, a “RNAi agent” for use in the compositions and methods of the disclosure is a double stranded RNA and is referred to herein as a “double stranded RNAi agent.” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., either a sense strand of a C9orf72 gene or an antisense strand of a C9orf72 gene. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

The dsRNA agents described herein can differ from (i.e., do not include) antisense oligonucleotides (ASOs) or gapmer antisense oligonucleotides (ASOs).

In some embodiments, any of the disclosed antisense oligonucleotide sequences described herein can be used alone as an ASO, ribozyme. The ASO can comprise 16-20 contiguous nucleotides from any of the described antisense oligonucleotide sequences. In some embodiments, an ASO targets the same region of a target RNA as any of the described dsRNAs. An ASO can down regulate a target by inducing RNase H endonuclease cleavage of a target RNA, by steric hindrance of ribosomal activity, by inhibiting 5′ cap formation, or by altering splicing. The ASO can be a gapmer or a morpholino. A “Gapmer” is oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” A gapmer can have 5′ and 3′ wings each having 2-6 nucleotides and a gap having 7-12 nucleotides. A gapmer can have a 3-10-3 configuration or a 5-10-5 configuration. All of the nucleotides of a gapmer can have phosphorothioate linkages, optionally with one or more chiral mesyl-phosphoramidate or methylphosponate linked nucleotides. The wing nucleotides can be, but are not limited to 2′-O-methoxyethyl (2′-MOE) modified nucleotides, LNA modified nucleotides, cET modified nucleotides or combinations thereof. The gap nucleotides can be deoxyribonucleotides. Any cytosine nucleotides in an ASO may be methyl-cytosines.

In general, a dsRNA molecule can include ribonucleotides, but as described in detail herein, each or both strands can also include one or more non-ribonucleotides, e.g., a deoxyribonucleotide, a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims.

In certain embodiments of the instant disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 15-36 base pairs in length, for example, about 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not, but can be covalently connected. In certain embodiments where the two strands are connected covalently by means other than an uninterrupted chain of nucleotides between the 3′-end of one strand and the 5′-end of the respective other strand forming the duplex structure, the connecting structure is referred to as a “linker” (though it is noted that certain other structures defined elsewhere herein can also be referred to as a “linker”). The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, an RNAi agent of the disclosure is a dsRNA, each strand of which independently comprises 19-23 nucleotides, that interacts with a target RNA sequence, e.g., a C9orf72 target mRNA sequence, to direct the cleavage of the target RNA.

In some embodiments, an iRNA of the invention is a dsRNA of 24-30 nucleotides that interacts with a target RNA sequence, e.g., a C9orf72 target mRNA sequence, to direct the cleavage of the target RNA.

As used herein, the term “nucleotide overhang” refers to at least one unpaired nucleotide that protrudes from the duplex structure of an RNAi agent, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the overhang on the sense strand or the antisense strand, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′ end of the antisense strand of the duplex. In certain embodiments, an extended overhang is present on the 5′ end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

In certain embodiments, at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, the entire contents of each of which are incorporated by reference herein). Such structures may include single-stranded extensions (on one or both sides of the molecule)as well as double-stranded extensions.

In certain embodiments, the 3′ end of the sense strand and the 5′ end of the antisense strand are joined by a polynucleotide sequence comprising ribonucleotides, deoxyribonucleotides or both, optionally wherein the polynucleotide sequence comprises a tetraloop sequence. In certain embodiments, the sense strand is 25-35 nucleotides in length.

A tetraloop may contain ribonucleotides, deoxyribonucleotides, modified nucleotides, and combinations thereof. Typically, a tetraloop has 4 to 5 nucleotides. In some embodiments, the loop comprises a sequence set forth as GAAA. In some embodiments, at least one of the nucleotide of the loop (GAAA) comprises a nucleotide modification. In some embodiments, the modified nucleotide comprises a 2′-modification. In some embodiments, the 2 ‘-modification is a modification selected from the group consisting of 2’-aminoethyl, 2′-fluoro, 2′-O-methyl, 2′-O-methoxyethyl, 2′-aminodiethoxymethanol, 2′-adem, and 2′-deoxy-2′-fhioro- -d-arabinonucleic acid. In some embodiments, all of the nucleotides of the loop are modified. In some embodiments, the G in the GAAA sequence comprises a 2′-OH. In some embodiments, each of the nucleotides in the GAAA sequence comprises a 2′-O-methyl modification. In some embodiments, each of the A in the GAAA sequence comprises a 2′-OH and the G in the GAAA sequence comprises a 2′-O-methyl modification. In preferred embodiments, In some embodiments, each of the A in the GAAA sequence comprises a 2′-O-methoxyethyl (MOE) modification and the G in the GAAA sequence comprises a 2′-O-methyl modification; or each of the A in the GAAA sequence comprises a 2′-adem modification and the G in the GAAA sequence comprises a 2′-O-methyl modification. See, e.g., PCT Publication No. WO 2020/206350, the entire contents of which are incorporated herein by reference.

An exemplary 2′ adem modified nucleotide is shown below:

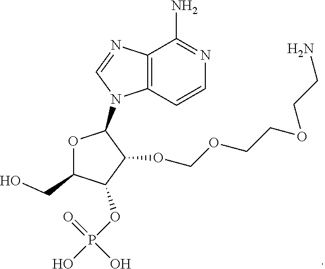 [SMILES: NCCOCCOCO[C@@H]1[C@H](OP(=O)(O)O)[C@@H](CO)O[C@H]1n1cnc2c(N)nccc21]

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” of an RNAi agent refers to the strand of the RNAi agent, e.g., a dsRNA, which includes a region that is substantially complementary to a target sequence, e.g., a C9orf72 mRNA.

As used herein, the term “region of complementarity” refers to the region on the antisense strand that is substantially complementary to a sequence, for example a target sequence, e.g., a C9orf72 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent. In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a C9orf72 gene, generally does not contain any mismatch within the central 13 nucleotides. The methods described herein or methods known in the art can be used to determine whether an RNAi agent containing a mismatch to a target sequence is effective in inhibiting the expression of a C9orf72 gene. Consideration of the efficacy of RNAi agents with mismatches in inhibiting expression of a C9orf72 gene is important, especially if the particular region of complementarity in a C9orf72 gene is known to have polymorphic sequence variation within the population. In some embodiments, the RNAi agent contains a single nucleotide mismatch with the target sequence wherein the mismatch occurs that the 3′ or 5′ terminus of the RNAi agent. The mismatch can be in the antisense strand, the sense strand or both the sense strand and the antisense strand. For an RNAi agent having a 3′ or 5′ terminal mismatch with the target RNA in both the sense strand and the antisense strand, the terminal nucleotides of the sense and antisense strand can for a base pair. Thus, for any of the described antisense or sense sequences disclosed herein, a 5′ or 3′ nucleotide may be substituted for a nucleotide that forms a mismatch with the target RNA.

As used herein, “substantially all of the nucleotides are modified” are largely but not wholly modified and can include not more than 5, 4, 3, 2, or 1 unmodified nucleotides.

The term “sense strand” or “passenger strand” of an RNAi agent refers to the strand of the RNAi agent that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary.” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° ° C. or 70ºC for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary.” “fully complementary” and “substantially complementary” herein can be used with respect to the base matching between the sense strand and the antisense strand of a dsRNA, or between the antisense strand of an RNAi agent and a target sequence, as will be understood from the context of their use.

As used herein, a polynucleotide that is “substantially complementary to at least part of” an RNA transcript refers to a polynucleotide that is substantially complementary to a contiguous portion of the RNA transcript of interest (e.g., a C9orf72 RNA, either sense strand or antisense strand). For example, a polynucleotide is complementary to at least a part of a C9orf72 RNA if the sequence is substantially complementary to a non-interrupted portion of an RNA.

Accordingly, in some embodiments, the antisense polynucleotides disclosed herein are fully complementary to the target C9orf72 sequence. In other embodiments, the antisense polynucleotides disclosed herein are substantially complementary to the target C9orf72 sequence and comprise a contiguous nucleotide sequence which is at least 80% complementary over its entire length to the equivalent region of the nucleotide sequence of any one of SEQ ID NOs: 1-4, 9, 11, 13, 15, 17 and 19 such as about 85%, about 90%, about 91%, about 92%, about 93%, about 94%, about 95%, about 96%, about 97%, about 98%, or about 99% complementary.

As described above, the large GGGGCC (G4C2) hexanucleotide repeat expansion (SEQ ID NO: 100) in the first intron of the C9orf72 gene between exons 1a and 1b and to be pathogenic can be bidirectionally transcribed. Accordingly, in some embodiments, antisense polynucleotides are disclosed herein that are complementary to the either strand of the C9orf72 gene. In other embodiments, the antisense polynucleotides disclosed herein are substantially complementary to the target C9orf72 sequence and comprise a contiguous nucleotide sequence which is at least 80% complementary over its entire length to the equivalent region of the nucleotide sequence of any one of SEQ ID NOs: 5-8, 10, 12, 14, 16, 18 or 20, such as about 85%, about 90%, about 91%, about 92%, about 93%, about 94%, about 95%, about 96%, about 97%, about 98%, or about 99% complementary. In some embodiments, the antisense polynucleotides disclosed herein are substantially complementary to a fragment of a target C9orf72 sequence and comprise a contiguous nucleotide sequence which is at least 80% complementary over its entire length to a fragment of SEQ ID NO:13 selected from the group of nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; and 27573717-27573739 of SEQ ID NO:13, such as about 85%, about 90%, about 91%, about 92%, about 93%, about 94%, about 95%, about 96%, about 97%, about 98%, or about 99% complementary.

In some embodiments, the antisense polynucleotides disclosed herein are substantially complementary to a fragment of a target C9orf72 sequence and comprise a contiguous nucleotide sequence which is at least 80% complementary over its entire length to a fragment of SEQ ID NO:1, such as nucleotides 1-23; 15-37; 33-55; 37-59; 62-84, or 69-91 of SEQ ID NO:1, such as about 85%, about 90%, about 91%, about 92%, about 93%, about 94%, about 95%, about 96%, about 97%, about 98%, or about 99% complementary. Ranges intermediate to the above recited ranges are also contemplated to be part of the disclosure.

In some embodiments, the antisense polynucleotides disclosed herein are substantially complementary to a fragment of a target C9orf72 sequence and comprise a contiguous nucleotide sequence which is at least 80% complementary over its entire length to a fragment of SEQ ID NO:15, such as nucleotides 5197-5219; 5223-5245; 5226-5248; 5227-5249; 5233-5255; 5248-5270; 5539-5561; 5547-5569; 5917-5939; 5936-5958; 5954-5976; 6008-6030; 6021-6043; 6036-6058; 6043-6065; and 6048-6070 of SEQ ID NO: 15, such as about 85%, about 90%, about 91%, about 92%, about 93%, about 94%, about 95%, about 96%, about 97%, about 98%, or about 99% complementary. Ranges intermediate to the above recited ranges are also contemplated to be part of the disclosure.

In some embodiments, the antisense polynucleotides disclosed herein are substantially complementary to a fragment of a target C9orf72 sequence and comprise a contiguous nucleotide sequence which is at least 80% complementary over its entire length to a fragment of SEQ ID NO:15, such as nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, such as about 85%, about 90%, about 91%, about 92%, about 93%, about 94%, about 95%, about 96%, about 97%, about 98%, or about 99% complementary. Ranges intermediate to the above recited ranges are also contemplated to be part of the disclosure.

In some embodiments, the antisense polynucleotides disclosed herein are substantially complementary to a fragment of a target C9orf72 sequence and comprise a contiguous nucleotide sequence which is at least 80% complementary over its entire length to a fragment of SEQ ID NO:1, such as nucleotides 15-52; 17-40; 32-59; 32-55; 35-59; 36-59; 58-87; 59-87; 59-84; or 64-87 of SEQ ID NO:1, such as about 85%, about 90%, about 91%, about 92%, about 93%, about 94%, about 95%, about 96%, about 97%, about 98%, or about 99% complementary. Ranges intermediate to the above recited ranges are also contemplated to be part of the disclosure.

In some embodiments, the antisense polynucleotides disclosed herein are substantially complementary to a fragment of a target C9orf72 sequence and comprise a contiguous nucleotide sequence which is at least 80% complementary over its entire length to a fragment of SEQ ID NO:13, such as nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, such as about 85%, about 90%, about 91%, about 92%, about 93%, about 94%, about 95%, about 96%, about 97%, about 98%, or about 99% complementary. Ranges intermediate to the above recited ranges are also contemplated to be part of the disclosure.

In other embodiments, the sense polynucleotides disclosed herein are substantially complementary to the target C9orf72 sequence and comprise a contiguous nucleotide sequence which is at least about 80% complementary over its entire length to any one of the sense strand nucleotide sequences in any one of any one of Tables 2, 3, 10A, 10C, 11, or 12, or a fragment of any one of the sense strand nucleotide sequences in any one of Tables 2, 3, 10A, 10C, 11, or 12 such as about 85%, about 90%, about 91%, about 92%, about 93%, about 94%, about 95%, about 96%, about 97%, about 98%, about 99%, or 100% complementary.

In other embodiments, the antisense polynucleotides disclosed herein are substantially complementary to the target C9orf72 sequence and comprise a contiguous nucleotide sequence which is at least about 80% complementary over its entire length to any one of the sense strand nucleotide sequences in any one of any one of Tables 5, 6, 10B, or 10D or a fragment of any one of the sense strand nucleotide sequences in any one of Tables 5, 6, 10B, or 10D such as about 85%, about 90%, about 91%, about 92%, about 93%, about 94%, about 95%, about 96%, about 97%, about 98%, about 99%, or 100% complementary.

In certain embodiments, the sense and antisense strands are selected from any one of duplexes AD-1446213.1; AD-1446217.1; AD-1446222.1; AD-1446234.1; AD-1446243.1; AD-1446246.1; AD-1446252.1; AD-1446259.1; AD-1446265.1; AD-1446268.1; AD-1446271.1; AD-1446279.1; AD-1446289.1; and AD-1446294.1.

In certain embodiments, the sense and antisense strands are selected from any one of duplexes AD-1446213.1; AD-1446246.1; and AD-1446268.1.

In certain embodiments, the sense and antisense strands are selected from any one of duplexes AD-1446073.1; AD-1446075.1; AD-1285246.2; AD-1446084.1; AD-1446087.1; AD-1446090.1, and AD-1446095.1.

In certain embodiments, the sense and antisense strands are selected from any one of duplexes AD-1446087.1 and AD-1446090.1.

In certain embodiments, the sense and antisense strands are selected from any one of duplexes AD-1285238.1; and AD-1285234.1.

In certain embodiments, the sense and antisense strands are selected from any one of duplexes AD-1285231.1. AD-1285232.1. AD-1285233.1. AD-1285235.1, AD-1285237.1. AD-1285239.1. AD-1285240.1. AD-1285242.1, AD-1285244.1; AD-1285243.1; AD-1285241.1; AD-1285236.1; AD-1446111.1; AD-1446117.1; AD-1446147.1; AD-1446157.1; AD-1446168.1; AD-1446180.1; AD-1446189.1; AD-1446196.1; AD-1446202.1; AD-1446205.1.

In certain embodiments, the sense and antisense strands are selected from any one of duplexes AD-1285231.1. AD-1285232.1. AD-1285233.1. AD-1285234.1, AD-1285235.1, AD-1285236.1, AD-1285237.1, AD-1285239.1, AD-1285240.1, AD-1285241.1, AD-1285242.1, and AD-1285243.1.

In other embodiments, the antisense polynucleotides disclosed herein are substantially complementary to the target C9orf72 sequence and comprise a contiguous nucleotide sequence which is at least about 80% complementary over its entire length to any one of the sense strand nucleotide sequences in any one of any one of Tables 8 or 9, or a fragment of any one of the sense strand nucleotide sequences in any one of Tables 8 or 9, such as about 85%, about 90%, about 91%, about 92%, about 93%, about 94%, about 95%, about 96%, about 97%, about 98%, about 99%, or 100% complementary.

As used herein, the phrase “inhibiting expression of C9orf72.” includes inhibiting the expression of a mature C9orf72 mRNA, knocking down or inhibiting the expression or reducing the level of a C9orf72 RNA containing a hexanucleotide-repeat in an intron, knocking down or inhibiting the expression or reducing the level of an antisense strand of a C9orf72 RNA containing a hexanucleotide-repeat. Knocking down or inhibiting the expression or reducing the level of a C9orf72 RNA containing a hexanucleotide-repeat includes inhibiting production of sense and antisense C9orf72-containing foci and/or inhibiting production of aberrant dipeptide-repeat (DPR) proteins (e.g., poly(glycine-alanine) or poly(GA) peptides, poly(glycine-proline) or poly(GP) peptides, poly(glycine-arginine) or poly(GR) peptides, poly(alanine-proline) or poly(PA) peptides, or poly(proline-arginine) or poly(PR) peptides). In some embodiments, the repeat-length-dependent formation of RNA foci, the sequestration of specific RNA-binding proteins, or the accumulation or aggregation of poly(glycine-alanine) peptides, poly(glycine-proline) peptides, poly(glycine-arginine) peptides, poly(alanine-proline) peptides, or poly(proline-arginine) peptides is inhibited or decreased by more than 50%, e.g., more than 55%, more than 60%, more than 65%, more than 70%, more than 75%, more than 80%, more than 85%, more than 90%, or more than 95%, and the expression of C9orf72 mature RNA is inhibited by less than 50%, e.g., less than 45%, less than 40%, less than 35%, less than 30%, less than 25%, less than 20%, less than 15%, less than 10% or less than 5%.

In one embodiment, at least partial suppression of the expression of a C9orf72 gene, is assessed by a reduction of the amount of a C9orf72 RNA, e.g., sense RNA transcript, antisense RNA transcript, total C9orf72 RNA transript, sense C9orf72 repeat-containing RNA transcript, and/or antisense C9orf72 repeat-containing RNA transcript, which can be isolated from or detected in a first cell or group of cells in which a C9orf72 gene is transcribed and which has or have been treated such that the expression of a C9orf72 gene is inhibited, as compared to a second cell or group of cells substantially identical to the first cell or group of cells but which has or have not been so treated (control cells). The degree of inhibition may be expressed in terms of:

$$\frac{( RNA in control cells ) - ( RNA in treated cells )}{RNA in control cells} \times 100 \%$$

The phrase “contacting a cell with an RNAi agent.” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the central nervous system (CNS), optionally via intrathecal, intravitreal or other injection, or to the bloodstream or the subcutaneous space, such that the agent will subsequently reach the tissue where the cell to be contacted is located. For example, the RNAi agent may contain or be coupled to a ligand, e.g., a lipophilic moiety or moieties as described below and further detailed, e.g., in PCT/US2019/031170, which is incorporated herein by reference, that directs or otherwise stabilizes the RNAi agent at a site of interest, e.g., the CNS. Combinations of in vitro and in vivo methods of contacting are also possible. For example, a cell may also be contacted in vitro with an RNAi agent and subsequently transplanted into a subject.

In one embodiment, contacting a cell with an RNAi agent includes “introducing” or “delivering the RNAi agent into the cell” by facilitating or effecting uptake or absorption into the cell. Absorption or uptake of an RNAi agent can occur through unaided diffusive or active cellular processes, or by auxiliary agents or devices. Introducing an RNAi agent into a cell may be in vitro or in vivo. For example, for in vivo introduction, an RNAi agent can be injected into a tissue site or administered systemically. In vitro introduction into a cell includes methods known in the art such as electroporation and lipofection. Further approaches are described herein below or are known in the art.

The term “lipophile” or “lipophilic moiety” broadly refers to any compound or chemical moiety having an affinity for lipids. One way to characterize the lipophilicity of the lipophilic moiety is by the octanol-water partition coefficient, logKow, where Kow is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its logKow exceeds 0. Typically, the lipophilic moiety possesses a logKow exceeding 1, exceeding 1.5, exceeding 2, exceeding 3, exceeding 4, exceeding 5, or exceeding 10. For instance, the logKow of 6-amino hexanol, for instance, is predicted to be approximately 0.7. Using the same method, the logKow of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., logKow) value of the lipophilic moiety.

Alternatively, the hydrophobicity of the double-stranded RNAi agent, conjugated to one or more lipophilic moieties, can be measured by its protein binding characteristics. For instance, in certain embodiments, the unbound fraction in the plasma protein binding assay of the double-stranded RNAi agent could be determined to positively correlate to the relative hydrophobicity of the double-stranded RNAi agent, which could then positively correlate to the silencing activity of the double-stranded RNAi agent.

In one embodiment, the plasma protein binding assay determined is an electrophoretic mobility shift assay (EMSA) using human serum albumin protein. An exemplary protocol of this binding assay is illustrated in detail in, e.g., PCT/US2019/031170. The hydrophobicity of the double-stranded RNAi agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded RNAi agent provides optimal hydrophobicity for the enhanced in vivo delivery of siRNA. In some embodiments, the lipophilic moiety facilitates or improves delivery of the RNAi agent to a neuronal cell, or a cell in a neuronal tissue, or a cell in a central nervous system tissue.

The term “lipid nanoparticle” or “LNP” is a vesicle comprising a lipid layer encapsulating a pharmaceutically active molecule, such as a nucleic acid molecule, e.g., an RNAi agent or a plasmid from which an RNAi agent is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a rat, or a mouse). In a preferred embodiment, the subject is a human, such as a human being treated or assessed for a disease, disorder, or condition that would benefit from reduction in levels of target C9orf72 RNA; a human at risk for a disease, disorder, or condition that would benefit from reduction in levels of target C9orf72 RNA; a human having a disease, disorder, or condition that would benefit from reduction in C9orf72 expression; or human being treated for a disease, disorder, or condition that would benefit from reduction in C9orf72 expression as described herein. In some embodiments, the subject is a female human. In other embodiments, the subject is a male human. In one embodiment, the subject is an adult subject. In one embodiment, the subject is a pediatric subject. In another embodiment, the subject is a juvenile subject, i.e., a subject below 20 years of age.

As used herein, the terms “treating” or “treatment” refer to a beneficial or desired result including, but not limited to, alleviation or amelioration of one or more signs or symptoms associated with expression of a C9orf72 hexanucleotide repeat expansion transcript or a dipeptide repeat product thereof, e.g., C9orf72-associated diseases, such as C9orf72-associated disease. “Treatment” can also mean prolonging survival as compared to expected survival in the absence of treatment.

The term “lower” in the context of the level of C9orf72 in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., the level of sense- or antisense-containing foci and/or the level of aberrant dipeptide repeat protein, e.g., a decrease of 50%, 55%, 60%, 65%, 70%, 75%, 80%, 85%, 90%, 95%, or more. In some embodiments, a decrease is no more than 50% for C9orf72 protein and/or C9orf72 mRNA level, e.g., no more than 50%, 45%, 40%, 35%, 30%, 25%. 20%, 15%, 10%, or 5%. “Lower” in the context of the level of C9orf72 in a subject is preferably down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in bodyweight between an obese individual and an individual having a weight accepted within the range of normal.

As used herein, “prevention” or “preventing.” when used in reference to a disease, disorder, or condition thereof, that would benefit from a reduction in expression of a C9orf72 hexanucleotide repeat expansion transcript or a dipeptide product thereof, refers to a reduction in the likelihood that a subject will develop a symptom associated with such a disease, disorder, or condition, e.g., a symptom of a C9orf72-associated disease. The failure to develop a disease, disorder, or condition, or the reduction in the development of a symptom associated with such a disease, disorder, or condition (e.g., by at least about 10% on a clinically accepted scale for that disease or disorder), or the exhibition of delayed symptoms delayed (e.g., by days, weeks, months or years) is considered effective prevention.

As used herein, the term “C9orf72-associated disease” or “C9orf72-associated disorder” includes any disease or disorder that would benefit from reduction in the expression and/or activity of C9orf72 hexanucleotide repeat expansion transcript. Exemplary C9orf72-associated diseases include those diseases in which subjects carry a hexanucleotide repeat (GGGGCC (SEQ ID NO: 100)) expansion in the intron between exons 1a and 1b in the C9orf72 gene, e.g., amyotrophic lateral sclerosis, frontotemporal dementia, Huntington's disease, e.g., Huntington-Like Syndrome Due To C9orf72 Expansions, parkinsonism, olivopontocerebellar degeneration, corticobasal syndrome, or Alzheimer's disease.

Normal G4C2 repeats are ˜25 units or less, and high penetrance disease alleles are typically greater than ˜60 repeat units, ranging up to more than 4,000 units; rarely, repeats between 47 and 60 segregate with disease in families. A repeat-primed PCR assay is typically used to detect smaller expansions (<80), but accurately sizing larger repeats requires other techniques (e.g. Southern blot hybridization) that provides an estimate of length.

Subjects having a GGGGCC (or G4C2) hexanucleotide expansion (SEQ ID NO: 100) in an intron of the C9orf72 gene can present as amyotrophic lateral sclerosis (ALS) or frontotemporal dementia (FTD) even in the same family and, therefore, the neurodegeneration associated with this expansion is referred to herein as “C9orf72 Amyotrophic lateral sclerosis/frontotemporal dementia” or C9orf72 ALS/FTD.” It is an autosomal dominant disease and is the most common form of familial ALS, accounting for about a third of ALS families and 5-10% of sporadic cases in an ALS clinic. It is also a common cause of FTD, explaining about one fourth of familial FTD. Age of symptom onset ranges from 30 to 70 years of age with a mean onset in the late 50s. C9orf72-mediated ALS most often resembles typical ALS, can be bulbar or limb onset, can progress rapidly (though not always) and can be associated with later cognitive symptoms. Thus, C9orf72-mediated ALS is evaluated and treated just as in any ALS patient. The pattern of C9orf72-mediated FTD most commonly is behavioral variant FTD, with the full range of behavioral and cognitive symptoms including disinhibition, apathy and executive dysfunction. Less commonly, C9orf72-mediated FTD presents semantic variant primary progressive aphasia (PPA) or nonfluent variant PPA, and, very rarely, can resemble corticobasal syndrome, progressive supranuclear palsy or an HD-like syndrome. Occasionally parkinsonian features are seen in C9orf72-mediated ALS or FTD.

Subjects may exhibit frontotemporal lobar degeneration (FTLD) characterized by progressive changes in behavior, executive dysfunction, and/or language impairment. Of the three FTLD clinical syndromes, behavioral variant FTD (bvFTD) is most often, but not exclusively, present. It is characterized by progressive behavioral impairment and a decline in executive function with predominant frontal lobe atrophy on brain MRI. Motor neuron disease, including upper or lower motor neuron dysfunction (or both) that may or may not fulfill criteria for the full ALS phenotype may also be present. Some degree of parkinsonism, which is present in many individuals with C9orf72-associated bvFTD, is typically of the akinetic-rigid type without tremor, and is levodopa unresponsive.

Huntington's disease-like syndromes (HD-like syndromes, or HDL syndromes) are a family of inherited neurodegenerative diseases that closely resemble Huntington's disease (HD) in that they typically produce a combination of chorea, cognitive decline or dementia and behavioral or psychiatric problems.

Subjects having Huntington disease-like syndrome due to C9orf72 expansions are characterized as having movement disorders, including dystonia, chorea, myoclonus, tremor and rigidity. Associated features are also cognitive and memory impairment, early psychiatric disturbances and behavioral problems. The mean age at onset is about 43 years (range 8-60). Early psychiatric and behavioral problems (including depression, apathy, obsessive behavior, and psychosis) are common. Cognitive symptoms present as executive dysfunction. Movement disorders are prominent: Parkinsonian features and pyramidal features may also be present. “Therapeutically effective amount,” as used herein, is intended to include the amount of an RNAi agent that, when administered to a subject having a C9orf72-associated disease, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount.” as used herein, is intended to include the amount of an RNAi agent that, when administered to a subject having a C9orf72-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. An RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials (including salts), compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations.

The term “sample.” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the brain (e.g., whole brain or certain segments of brain, e.g., striatum, or certain types of cells in the brain, such as, e.g., neurons and glial cells (astrocytes, oligodendrocytes, microglial cells)). In some embodiments, a “sample derived from a subject” refers to blood drawn from the subject or plasma or serum derived therefrom. In further embodiments, a “sample derived from a subject” refers to brain tissue (or subcomponents thereof) or retinal tissue (or subcomponents thereof) derived from the subject

### II. RNAi Agents of the Disclosure

As described elsewhere herein, mutations in C9orf72 have been linked to familial frontotemporal dementia (FTD) and amyotrophic lateral sclerosis (ALS). The mutations are the result of expansion of G4C2 (SEQ ID NO: 100) hexanucleotide repeats located within the intron between exon 1A and exon 1B of the C9orf72 gene. The hexanucleotide repeats may be translated through a non-AUG-initiated mechanism. Accumulation of the repeat expansion-containing RNA (target RNA) or translation of the repeat sequences may cause or contribute to FTD and/or ALS or disease symptoms associated with FTD and/or ALS.

Accordingly, the present invention provides dsRNA agents that selectively and efficiently decrease expression of C9orf72-related expression products, RNA and/or translated polypeptides, associated with the hexanucleotide repeat expansions. In some embodiments, the dsRNA agents target (e.g., selectively target) the hexanucleotide-repeat-containing RNA (target RNA) and knock down the target RNA and polypeptides expressed from the hexanucleotide-repeat-containing RNA. The dsRNA agents may be used in methods for therapeutic treatment and/or prevention of signs or symptoms associated with FTD and/or ALS, including, but not limited to, repeat-length-dependent formation of RNA foci, sequestration of specific RNA-binding proteins, and accumulation and aggregation of dipeptide repeat proteins (e.g., poly(glycine-alanine), poly(glycine-proline), poly(glycine-arginine), poly(alanine-proline), and poly(proline-arginine)) resulting from repeat-associated non-AUG (AUG) translation in neurons. The dsRNA agents may be used in methods for therapeutic treatment and/or prevention of signs or symptoms associated with FTD and/or ALS, including, but not limited to, signs and symptoms of motor neuron disease and signs and symptoms of dementia. Signs and symptoms of motor neuron disease can include, for example, tripping, dropping things, abnormal fatigue of the arms and/or legs, slurred speech, muscle cramps and twitches, uncontrollable periods of laughing or crying, and trouble breathing. Signs and symptoms of dementia can include, for example, behavioral changes, personality changes, speech and language problems, and movement-related problems. Such methods comprise administration of one or more dsRNA agents as described herein to a subject (e.g., a human or animal subject).

The dsRNA agents described herein may stop or reduce the accumulation of repeat-containing C9orf72 RNA (e.g., assayed as RNA foci) and thereby prevent the synthesis of dipeptide repeat proteins by RAN translation.

In some embodiments, the dsRNA agents of the invention target mature C9orf72 mRNAs (i.e., mRNAs in which introns have been spliced out). In other embodiments, the dsRNA agents of the invention target C9orf72 RNAs containing an intron, such as intron 1A (i.e., sense or antisense RNAs in which introns have not been spliced out, RNA regions spliced out of a precursor mRNA, or alternatively spliced RNAs).

A dsRNA includes two RNA strands that are complementary and hybridize to form a duplex structure under conditions in which the dsRNA will be used. In some embodiments, one strand of a dsRNA (the antisense strand) includes a region of complementarity that is substantially complementary, and generally fully complementary, to a target sequence. The target sequence can be derived from the sequence of an RNA formed during the expression of a C9orf72 gene. The other strand (the sense strand) includes a region that is complementary to the antisense strand, such that the two strands hybridize and form a duplex structure when combined under suitable conditions. In some embodiments, one strand of a dsRNA (the sense strand) includes a region of complementarity that is substantially complementary, and generally fully complementary, to a target sequence derived from the antisense sequence of an RNA formed during the expression of a C9orf72 gene. The other strand (the antisense strand) includes a region that is complementary to the sense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain preferred embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 base pairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is 15 to 23 nucleotides in length, 19 to 23 nucleotides in length, or 25 to 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an RNAi agent useful to target C9orf72 expression is not generated in the target cell by cleavage of a larger dsRNA.

A dsRNA as described herein can further include one or more single-stranded nucleotide overhangs e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

Regardless of the method of synthesis, the siRNA preparation can be prepared in a solution (e.g., an aqueous or organic solution) that is appropriate for formulation. For example, the siRNA preparation can be precipitated and redissolved in pure double-distilled water, and lyophilized. The dried siRNA can then be resuspended in a solution appropriate for the intended formulation process.

In certain embodiments, the dsRNA agents of the invention target a C9orf72 target RNA comprising a hexanucleotide repeat comprising multiple contiguous copies, for example, a C9orf72 target RNA with a pathogenic hexanucleotide repeat expansion (having, for example, at least about 30, at least about 35, at least about 40, at least about 50, at least about 60, at least about 70, at least about 80, at least about 100, at least about 200, at least about 300, at least about 400, or at least about 500 copies of the hexanucleotide repeat).

In one aspect, a dsRNA of the disclosure includes at least two nucleotide sequences, a sense sequence and an antisense sequence. The sense strand sequence for C9orf72 may be selected from the group of sequences provided in any one of Tables 2, 3, 5, 6, 8, 9, 10A, 10B, 10C, 10D, 11, and 12 and the corresponding nucleotide sequence of the antisense strand of the sense strand may be selected from the group of sequences of any one of Tables 2, 3, 5, 6, 8, 9, 10A, 10B, 10C, 10D, 11, and 12. In this aspect, one of the two sequences is complementary to the other of the two sequences, with one of the sequences being substantially complementary to a sequence of an RNA generated in the expression of a C9orf72 gene locus. As such, in this aspect, a dsRNA will include two oligonucleotides, where one oligonucleotide is described as the sense strand (passenger strand) in any one of Tables 2, 3, 5, 6, 8, 9, 10A, 10B, 10C, 10D, 11, and 12 and the second oligonucleotide is described as the corresponding antisense strand (guide strand) of the sense strand in any one of Tables 2, 3, 5, 6, 8, 9, 10A, 10B, 10C, 10D, 11, and 12.

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences in Tables 2 and 5 are described as modified or conjugated sequences, the RNA of the RNAi agent of the disclosure e.g., a dsRNA of the disclosure, may comprise any one of the sequences set forth in any one of Tables 2, 3, 5, 6, 8, 9, 10A, 10B, 10C, 10D, 11, and 12 that is un-modified, un-conjugated, or modified or conjugated differently than described therein.

The skilled person is well aware that dsRNAs having a duplex structure of about 20 to 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of a C9orf72 gene by not more than 10, 15, 20, 25, or 30% inhibition from a dsRNA comprising the full sequence using the in vitro assay with, e.g., Bc(2)c cells and a 10 nM concentration of the RNA agent and the PCR assay as provided in the examples herein, are contemplated to be within the scope of the present disclosure.

In addition, the RNAs described herein identify a site(s) in a C9orf72 transcript that is susceptible to RISC-mediated cleavage. As such, the present disclosure further features RNAi agents that target within this site(s). As used herein, an RNAi agent is said to target within a particular site of an RNA transcript if the RNAi agent promotes cleavage of the transcript anywhere within that particular site. Such an RNAi agent will generally include at least about 15 contiguous nucleotides, preferably at least 19 nucleotides, from one of the sequences provided herein coupled to additional nucleotide sequences taken from the region contiguous to the selected sequence in a C9orf72 gene.

The dsRNA agents disclosed herein inhibit expression of the C9orf72 target RNA comprising the hexanucleotide repeat. Inhibiting expression includes any level of inhibition (e.g., partial inhibition of expression). For example, the dsRNA agents may inhibit expression of the C9orf72 target RNA comprising the hexanucleotide repeat by at least about 10%, at least about 20%, at least about 30%, at least about 40%, at least about 50%, at least about 60%, at least about 70%, at least about 80%, or at least about 90% (or to a point where the C9orf72 target RNA is undetectable). For example, these levels of inhibition can be within 24-48 hours after administration to a cell expressing the C9orf72 target RNA comprising the hexanucleotide repeat. The decrease can be, for example, relative to the cell before treatment with dsRNA agent or relative to a control cell that was not treated with the dsRNA agent.

The dsRNA agents disclosed herein may also, for example, selectively reduce the level of or inhibit expression of the C9orf72 target RNA comprising the intronic hexanucleotide repeat relative to expression of a mature C9orf72 messenger RNA. A mature C9orf72 messenger RNA in this context is a C9orf72 RNA transcript that has been spliced and processed. A mature C9orf72 messenger RNA consists exclusively of exons and has all introns removed. A dsRNA agent may selectively inhibit expression of the C9orf72 target RNA comprising the intronic hexanucleotide repeat relative to expression of a mature C9orf72 messenger RNA if the relative decrease in expression of the C9orf72 target RNA is greater than the relative decrease in expression of a mature C9orf72 messenger RNA after administration of the dsRNA agent to a cell expressing the C9orf72 target RNA. For example, dsRNA agents may inhibit expression of the mature C9orf72 messenger RNA by less than about 50%, less than about 40%, less than about 30%, less than about 20%, less than about 10%, or less than about 5% (or, for example, does not have any statistically significant or functionally significant effect on expression). For example, these levels of inhibition can be within 24-48 hours after administration to a cell expressing the mature C9orf72 messenger RNA.

The dsRNA agents disclosed herein can also, for example, reduce dipeptide repeat protein synthesis or dipeptide repeat protein levels in a cell (e.g., within 24-48 hours after administration to the cell). For example, the dsRNA agent may reduce dipeptide repeat protein synthesis or dipeptide repeat protein levels by at least about 10%, at least about 20%, at least about 30%, at least about 40%, at least about 50%, at least about 60%, at least about 70%, at least about 80%, or at least about 90%. The decrease can be, for example, relative to the cell before treatment with dsRNA agent or relative to a control cell that was not treated with the dsRNA agent.

According to certain aspects of the invention, an iRNA agent may be designed to target a hotspot region of any of the target RNAs described herein, including any identified portions of a target RNA (e.g., a particular exon). As used herein, a hotspot region may refer to an approximately 19-200, 19-150, 19-100, 19-75, 19-50, 21-200, 21-150, 21-100, 21-75, 21-50, 50-200, 50-150, 50-100, 50-75, 75-200, 75-150, 75-100, 100-200, or 100-150 nucleotide region of a target RNA sequence for which targeting using RNAi agents provides an observably higher probability of efficacious silencing relative to targeting other regions of the same target RNA. According to certain aspects of the invention, a hotspot region may comprise a limited region of the target RNA, and in some cases, a substantially limited region of the target, including for example, less than half of the length of the target RNA, such as about 5%, 10%, 15%, 20%, 25%, or 30% of the length of the target RNA. Conversely, the other regions against which a hotspot is compared may cumulatively comprise at least a majority of the length of the target RNA. For example, the other regions may cumulatively comprise at least about 60%, or at least about 70%, or at least about 80%, or at least about 90%, or at least about 95% of the length of the target RNA.

Compared regions of the target RNA may be empirically evaluated for identification of hotspots using efficacy data obtained from in vitro or in vivo screening assays. For example, RNAi agents targeting various regions that span a target RNA may be compared for frequency of efficacious iRNA agents (e.g., the amount by which target gene expression is inhibited, such as measured by mRNA expression or protein expression) that bind each region. In general, a hotspot can be recognized by observing clustering of multiple efficacious RNAi agents that bind to a limited region of the RNA target. A hotspot may be sufficiently characterized as such by observing efficacy of iRNA agents which cumulatively span at least about 60% of the target region identified as a hotspot, such as about 70%, about 80%, about 90%, or about 95% or more of the length of the region, including both ends of the region (i.e. at least about 60%, 70%, 80%, 90%, or 95% or more of the nucleotides within the region, including the nucleotides at each end of the region, were targeted by an iRNA agent). According to some aspects of the invention, an iRNA agent which demonstrates at least about 50%, 55%, 60%, 65%, 70%, 75%, 80%, 85%, 90%, or 95% inhibition over the region (e.g., no more than about 50%, 45%, 40%, 35%, 30%, 25%, 20%, 15%, 10%, or 5% mRNA remaining) may be identified as efficacious.

Amenibility to targeting of RNA regions may also be assessed using quantitative comparison of inhibition measurements across different regions of a defined size (e.g. 25, 30, 40, 50, 60, 70, 80, 90, or 100, 110, 120, 130, 140, 150, 160, 170, 180, 190, or 200 nts). For example, an average level of inhibition may be determined for each region and the averages of each region may be compared. The average level of inhibition within a hotspot region may be substantially higher than the average of averages for all evaluated regions. According to some aspects, the average level of inhibition in a hotspot region may be at least about 10%, 20%, 30%, 40%, or 50% higher than the average of averages. According to some aspects, the average level of inhibition in a hotspot region may be at least about 1.0, 1.1, 1.2, 1.3, 1.4, 1.5 1.6, 1.7, 1.8, 1.9, or 2.0 standard deviations above the average of averages. The average level of inhibition may be higher by a statistically significant (e.g., p<0.05) amount. According to some aspects, each inhibition measurement within a hotspot region may be above a threshold amount (e.g., at or below a threshold amount of mRNA remaining). According to some aspects, each inhibition measurement within the region may be substantially higher than an average of all inhibition measurements across all the measured regions. For example, each inhibition measurement in a hotspot region may be at least about 10%, 20%, 30%, 40%, or 50% higher than the average of all inhibition measurements. According to some aspects, each inhibition measurement may be at least about 1.0, 1.1, 1.2, 1.3, 1.4, 1.5 1.6, 1.7, 1.8, 1.9, or 2.0 standard deviations above the average of all inhibition measurements. Each inhibition measurement may be higher by a statistically significant (e.g., p<0.05) amount than the average of all inhibition measurements. A standard for evaluating a hotspot may comprise various combinations of the above standards where compatible (e.g., an average level of inhibition of at least about a first amount and having no inhibition measurements below a threshold level of a second amount, lesser than the first amount).

It is therefore expressly contemplated that any iRNA agent, including the specific exemplary iRNA agents described herein, which targets a hotspot region of a target RNA, may be preferably selected for inducing RNA interference of the target mRNA as targeting such a hotspot region is likely to exhibit a robust inhibitory response relative to targeting a region which is not a hotspot region. RNAi agents targeting target sequences that substantially overlap (e.g., by at least about 70%, 75%, 80%, 85%, 90%, 95% of the target sequence length) or, preferably, that reside fully within the hotspot region may be considered to target the hotspot region. Hotspot regions of the RNA target(s) of the instant invention may include any region for which the data disclosed herein demonstrates higher frequency of targeting by efficacious RNAi agents, including by any of the standards described elsewhere herein, whether or not the range(s) of such hotspot region(s) are explicitly specified.

In various embodiments, a dsRNA agent of the present invention targets a hotspot region. In one embodiment, the hotspot region comprises the nucleotide sequence of any one of the sequences selected from SEQ ID Nos. 21-47 and 51-93. In another embodiment, the hotspot region comprises nucleotides 220-256, 220-266, 200-290 of SEQ ID NO: 13.

### III. Modified RNAi Agents of the Disclosure

In one embodiment, the nucleotide of the RNAi agent of the disclosure e.g., a dsRNA, is un-modified, and does not comprise, e.g., chemical modifications or conjugations known in the art and described herein. In preferred embodiments, the nucleotide of an RNAi agent of the disclosure, e.g., a dsRNA, is chemically modified to enhance stability or other beneficial characteristics. In certain embodiments of the disclosure, substantially all of the nucleotides of an RNAi agent of the disclosure are modified. In other embodiments of the disclosure, all of the nucleotides of an RNAi agent of the disclosure are modified. RNAi agents of the disclosure in which “substantially all of the nucleotides arc modified” are largely but not wholly modified and can include not more than 5, 4, 3, 2, or unmodified nucleotides. In still other embodiments of the disclosure, RNAi agents of the disclosure can include not more than 5, 4, 3, 2 or 1 modified nucleotides.

The nucleic acids featured in the disclosure can be synthesized or modified by methods well established in the art, such as those described in “Current protocols in nucleic acid chemistry.” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to. RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages. 2′-5′-linked analogs of these, and those having inverted polarity wherein the adjacent pairs of nucleoside units are linked 3′-5′ to 5′-3′ or 2′-5′ to 5′-2′.

Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothioate groups present in the agent. Agents in which substantially all of the phosphodiester and/or phosphorothioate linkages have a sodium counterion include not more than 5, 4. 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothioate groups present in the agent.

Representative U.S. patents that teach the preparation of the above phosphorus-containing linkages include, but are not limited to, U.S. Pat. Nos. 3.687.808; 4.469.863; 4.476.301; 5.023,243; 5.177.195; 5.188.897; 5.264.423; 5.276.019; 5.278.302; 5.286.717; 5.321.131; 5.399.676; 5.405.939; 5.453.496; 5.455.233; 5.466.677; 5.476.925; 5.519.126; 5.536.821; 5.541.316; 5.550.111; 5.563.253; 5.571.799; 5.587.361; 5.625.050; 6.028.188; 6.124.445; 6.160.109; 6.169.170; 6.172.209; 6, 239.265; 6.277.603; 6.326.199; 6.346.614; 6.444.423; 6.531.590; 6.534.639; 6.608.035; 6.683.167; 6.858.715; 6.867.294; 6.878.805; 7.015.315; 7.041.816; 7.273.933; 7.321.029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methylencimino and methylenchydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034.506; 5,166.315; 5,185,444; 5,214,134; 5,216,141; 5.235,033; 5,64.562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677.437; and, 5,677,439, the entire contents of each of which are hereby incorporated herein by reference.

In other embodiments, suitable RNA mimetics are contemplated for use in RNAi agents, in which both the sugar and the internucleoside linkage, i.e., the backbone, of the nucleotide units are replaced with novel groups. The base units are maintained for hybridization with an appropriate nucleic acid target compound. One such oligomeric compound, a RNA mimetic that has been shown to have excellent hybridization properties, is referred to as a peptide nucleic acid (PNA). In PNA compounds, the sugar backbone of an RNA is replaced with an amide containing backbone, in particular an aminocthylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5.714,331; and 5,719,262, the entire contents of each of which are hereby incorporated herein by reference. Additional PNA compounds suitable for use in the RNAi agents of the disclosure are described in, for example, in Nielsen et al., Science, 1991, 254, 1497-1500.

Some embodiments featured in the disclosure include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The RNAi agents, e.g., dsRNAs, featured herein can include one of the following at the 2′-position: OH; F; O-. S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]_{mC}H₃, O(CH₂).ₙOCH₃, O(CH₂), NH₂, O(CH₂)ₙ CH₃, O(CH₂), ONH₂, and O(CH₂)ON[(CH₂), CH₃)]2, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCH₃, SOCH₃, SO_{2C}H₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent, or a group for improving the pharmacodynamic properties of an RNAi agent, and other substituents having similar properties. In some embodiments, the modification includes a 2′-methoxyethoxy (2′-O—CH_{2C}H₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminocthoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′—OCH₃), 2′-aminopropoxy (2′—OCH_{2C}H_{2C}H₂NH₂), 2′-O-hexadecyl, and 2′-fluoro (2′-F). Similar modifications can also be made at other positions on the RNA of an RNAi agent, particularly the 3′ position of the sugar on the 3′ terminal nucleotide or in 2′-5′ linked dsRNAs and the 5′ position of 5′ terminal nucleotide. RNAi agents can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5.319,080; 5.359,044; 5,393,878; 5,446,137; 5,466,786; 5.514,785; 5,519,134; 5,567,811; 5,576,427; 5,591,722; 5,597,909; 5,610,300; 5,627,053; 5,639,873; 5,646.265; 5,658,873; 5,670,633; and 5,700,920, certain of which are commonly owned with the instant application. The entire contents of each of the foregoing are hereby incorporated herein by reference.

An RNAi agent of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P, ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° ° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7.045,610; 7,427,672; and 7,495,088, the entire contents of each of which are hereby incorporated herein by reference.

An RNAi agent of the disclosure can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

An RNAi agent of the disclosure can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (sec, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative US Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and ß-D-ribofuranose (see WO 99/14226).

An RNAi agent of the disclosure can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge. In one embodiment, a constrained ethyl nucleotide is in the S conformation referred to herein as “S-cEt.”

An RNAi agent of the disclosure may also include one or more “conformationally restricted nucleotides” (“CRN”). CRN are nucleotide analogs with a linker connecting the C₂′ and C₄′ carbons of ribose or the C₃ and -C₅′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US 2013/0190383; and WO 2013/036868, the entire contents of each of which are hereby incorporated herein by reference.

In some embodiments, an RNAi agent of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C₁′-C₄′ have been removed (i.e. the covalent carbon-oxygen-carbon bond between the C₁′ and C₄′ carbons). In another example, the C₂′-C₃′ bond (i.e. the covalent carbon-carbon bond between the C₂′ and C₃′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C₆-NHAc), N-(caproyl-4-hydroxyprolinol (Hyp-C₆), N-(acetyl-4-hydroxyprolinol (Hyp-NHAc), thymidine-2′-O-deoxythymidine (ether), N-(aminocaproyl)-4-hydroxyprolinol (Hyp-C₆-amino), 2-docosanoyl-uridine-3′-phosphate, inverted 2′-deoxy-modified ribonucleotide, such as inverted dT(idT), inverted dA (idA), and inverted abasic 2′-deoxyribonucleotide (iAb) and others. Disclosure of this modification can be found in WO 2011/005861.

In one example, the 3′ or 5′ terminal end of a oligonucleotide is linked to an inverted 2′-deoxy-modified ribonucleotide, such as inverted dT(idT), inverted dA (idA), or a inverted abasic 2′-deoxyribonucleotide (iAb). In one particular example, the inverted 2′-deoxy-modified ribonucleotide is linked to the 3′end of an oligonucleotide, such as the 3′-end of a sense strand described herein, where the linking is via a 3′-3′ phosphodiester linkage or a 3′-3′-phosphorothioate linkage.

In another example, the 3′-end of a sense strand is linked via a 3′-3′-phosphorothioate linkage to an inverted abasic ribonucleotide (iAb). In another example, the 3′-end of a sense strand is linked via a 3′-3′-phosphorothioate linkage to an inverted dA (idA).

In another example, the 5′-end of a sense strand is linked via a 3′-3′-phosphorothioate linkage to an inverted abasic ribonucleotide (iAb). In another example, the 5′-end of a sense strand is linked via a 3′-3′-phosphorothioate linkage to an inverted dA (idA).

In another example, the 3′- and 5′-ends of a sense strand are linked via a 3′-3′-phosphorothioate linkages to inverted abasic ribonucleotides (iAb). In another example, the 3′- and 5′-ends of a sense strand are linked via a 3′-3′-phosphorothioate linkages to inverted dAs (idA).

In one particular example, the inverted 2′-deoxy-modified ribonucleotide is linked to the 3′end of an oligonucleotide, such as the 3′-end of a sense strand described herein, where the linking is via a 3′-3′ phosphodiester linkage or a 3′-3′-phosphorothioate linkage.

In another example, the 3′-terminal nucleotides of a sense strand is an inverted dA (idA) and is linked to the preceding nucleotide via a 3′-3′-linkage (e.g., 3′-3′-phosphorothioate linkage).

Other modifications of an RNAi agent of the disclosure include a 5′ phosphate or 5′ phosphate mimic, e.g., a 5′-terminal phosphate or phosphate mimic on the antisense strand of an RNAi agent. Suitable phosphate mimics are disclosed in, for example US 2012/0157511, the entire contents of which are incorporated herein by reference.

A. Modified RNAi agents Comprising Motifs of the Disclosure

In certain aspects of the disclosure, the double-stranded RNAi agents of the disclosure include agents with chemical modifications as disclosed, for example, in WO 2013/075035, the entire contents of which are incorporated herein by reference. As shown herein and in WO 2013/075035, a superior result may be obtained by introducing one or more motifs of three identical modifications on three consecutive nucleotides into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally conjugated with a lipophilic ligand, e.g., a C16 ligand, for instance on the sense strand. The RNAi agent may be optionally modified with a (S)-glycol nucleic acid (GNA) modification, for instance on one or more residues of the antisense strand. The resulting RNAi agents present superior gene silencing activity.

Accordingly, the disclosure provides double stranded RNAi agents capable of inhibiting the expression of a target gene (i.e., a C9orf72 gene) in vivo. The RNAi agent comprises a sense strand and an antisense strand. Each strand of the RNAi agent may be 15-30 nucleotides in length. For example, each strand may be 16-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length. 19-23 nucleotides in length, 19-21 nucleotides in length, 21-25 nucleotides in length, or 21-23 nucleotides in length. In certain embodiments, each strand is 19-23 nucleotides in length.

The sense strand and antisense strand typically form a duplex double stranded RNA (“dsRNA”), also referred to herein as an “RNAi agent.” The duplex region of an RNAi agent may be 15-30 nucleotide pairs in length. For example, the duplex region can be 16-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length. In preferred embodiments, the duplex region is 19-21 nucleotide pairs in length.

In one embodiment, the RNAi agent may contain one or more overhang regions or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In preferred embodiments, the nucleotide overhang region is 2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F. 2′-O-methyl, thymidine (T), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′ end. The antisense strand contains at least one motif of three 2′-O-methyl modifications on three consecutive nucleotides at positions 11, 12, 13 from the 5′ end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′ end. The antisense strand contains at least one motif of three 2′-O-methyl modifications on three consecutive nucleotides at positions 11, 12, 13 from the 5′ end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′ end; the antisense strand contains at least one motif of three 2′-O-methyl modifications on three consecutive nucleotides at positions 11, 12, 13 from the 5′ end, wherein one end of the RNAi agent is blunt, while the other end comprises a 2 nucleotide overhang. Preferably, the 2 nucleotide overhang is at the 3′-end of the antisense strand. When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (e.g., a lipophilic ligand, optionally a C16 ligand).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3 ‘ terminal nucleotide of antisense strand is unpaired with sense strand, and up to 6 consecutive 3’ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1˜4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two, or three nucleotides in the duplex region.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U. G:U. I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense or antisense strand.

In one embodiment, the sense strand sequence may be represented by formula (I):

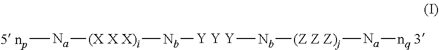

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and nq independently represent an overhang nucleotide;
wherein N_{b} and Y do not have the same modification; and
XXX, YYY and ZZZ each independently represent one motif of three identical modifications on three consecutive nucleotides. Preferably YYY is all 2′-F modified nucleotides.

In one embodiment, the Nₐ or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11,12 or 11, 12, 13) of—the sense strand, the count starting from the 1st nucleotide, from the 5′-end; or optionally, the count starting at the 1st paired nucleotide within the duplex region, from the 5′-end.

In one embodiment, i is 1 and j is 0, or i is 0 and j is 1, or both i and j are 1. The sense strand can therefore be represented by the following formulas:

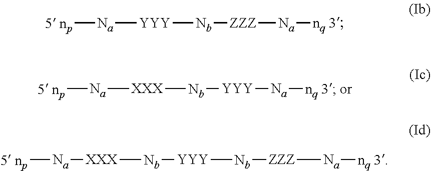

When the sense strand is represented by formula (Ib), N_{b} represents an oligonucleotide sequence comprising 0-10, 0-7, 0-5, 0-4, 0-2 or 0 modified nucleotides.

Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

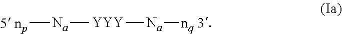

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

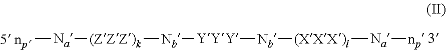

wherein:
k and I are each independently 0 or 1;
p′ and q′ are each independently 0-6;
   each Nₐ′ independently represents an oligonucleotide sequence comprising 0-25 modified nucleotides, each sequence comprising at least two differently modified nucleotides;
   each N_{b}′ independently represents an oligonucleotide sequence comprising 0-10 modified nucleotides;
   each nₚ′ and nq′ independently represent an overhang nucleotide;
   wherein No′ and Y′ do not have the same modification;
   and X′X′X′, Y′Y′Y′ and Z′Z′Z′ each independently represent one motif of three identical modifications on three consecutive nucleotides.
   In one embodiment, the Nₐ′ or N_{b}′ comprise modifications of alternating pattern.

The Y′Y′Y′ motif occurs at or near the cleavage site of the antisense strand. For example, when the RNAi agent has a duplex region of 17-23nucleotidein length, the Y′Y′Y′ motif can occur at positions 9, 10, 11; 10, 11, 12; 11, 12, 13; 12, 13, 14; or 13, 14, 15 of the antisense strand, with the count starting from the 1st nucleotide, from the 5′-end; or optionally, the count starting at the 1st paired nucleotide within the duplex region, from the 5′-end. Preferably, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and I are 1.

The antisense strand can therefore be represented by the following formulas:

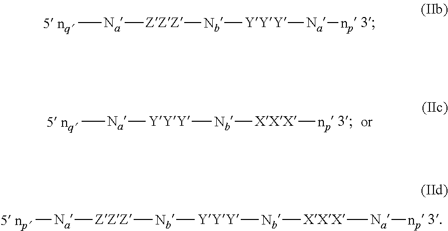

When the antisense strand is represented by formula (IIb), No′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and 1 is 0 and the antisense strand may be represented by the formula:

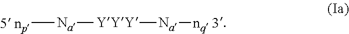

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, 1,5-anhydrohexitol (HNA), cyclohexenyl (CeNA), 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1st nucleotide from the 5′-end, or optionally, the count starting at the 1st paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1st nucleotide from the 5′-end, or optionally, the count starting at the 1st paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the RNAi agents for use in the methods of the disclosure may comprise a sense strand and an antisense strand, each strand having 14 to 30 nucleotides, the RNAi duplex represented by formula (III):

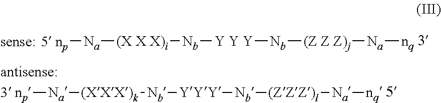

wherein:
i, j, k, and I are each independently 0 or 1;
p. p′, q, and q′ are each independently 0-6;
each Nₐ and Nₐ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein
each nₚ′, nₚ, nq′, and nq, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and I are 0; or both k and I are 1.

Exemplary combinations of the sense strand and antisense strand forming an RNAi duplex include the formulas below:

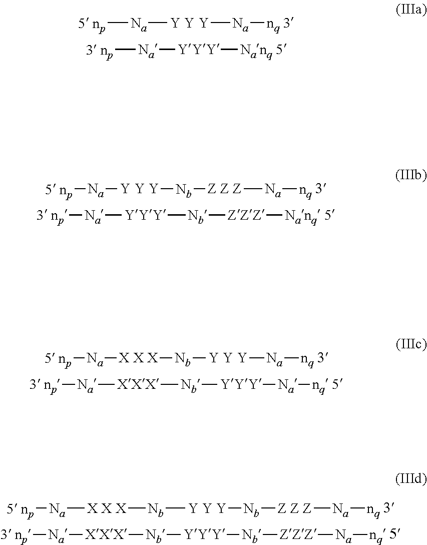

When the RNAi agent is represented by formula (IIIa), each Nₐ independently represents an oligonucleotide sequence comprising 2-20, 2-15, or 2-10 modified nucleotides.

When the RNAi agent is represented by formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as formula (IIIc), each No, N_{b}′ independently represents an oligonucleotide sequence comprising 0-10, 0-7, 0-10, 0-7, 0-5, 0-4, 0-2 or Omodified nucleotides. Each Nₐ independently represents an oligonucleotide sequence comprising 2-20, 2-15, or 2-10 modified nucleotides.

When the RNAi agent is represented as formula (IIId), each No, N_{b}′ independently represents an oligonucleotide sequence comprising 0-10, 0-7, 0-10, 0-7, 0-5, 0-4, 0-2 or 0 modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern.

In one embodiment, when the RNAi agent is represented by formula (IIId), the Nₐ modifications are 2′-O-methyl or 2′-fluoro modifications. In another embodiment, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet another embodiment, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more C16 (or related) moieties attached through a bivalent or trivalent branched linker (described below). In another embodiment, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more lipophilic, e.g., C16 (or related) moictics, optionally attached through a bivalent or trivalent branched linker.

In one embodiment, when the RNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more lipophilic, e.g., C16 (or related) moieties attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to a ligand. Each of the agents can target the same gene or two different genes; or each of the agents can target same gene at two different target sites.

Various publications describe multimeric RNAi agents that can be used in the methods of the disclosure. Such publications include WO2007/091269, WO2010/141511, WO2007/117686, WO2009/014887, and WO2011/031520; and U.S. Pat. No. 7,858,769, the entire contents of each of which are hereby incorporated herein by reference.

In certain embodiments, the compositions and methods of the disclosure include a vinyl phosphonate (VP) modification of an RNAi agent as described herein. In exemplary embodiments, a vinyl phosphonate of the disclosure has the following structure:

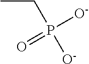

In exemplary embodiments, a 5′ vinyl phosphonate modified nucleotide of the disclosure has the structure:

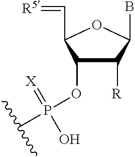

wherein X is O or S;
R is hydrogen, hydroxy, fluoro, or C₁₋₂₀alkoxy (e.g., methoxy or n-hexadecyloxy);
R⁵′ is ═C(H)—P(O)(OH), and the double bond between the C5′ carbon and R⁵′ is in the E or Z orientation (e.g., E orientation); and
B is a nucleobase or a modified nucleobase, optionally where B is adenine, guanine, cytosine, thymine, or uracil.

In one embodiment, R⁵′ is ═C(H)—P(O)(OH), and the double bond between the C5′ carbon and R⁵′ is in the E orientation. In another embodiment, R is methoxy and R⁵′ is ═C(H)—P(O)(OH)2 and the double bond between the C5′ carbon and R⁵′ is in the E orientation. In another embodiment, X is S, R is methoxy, and RS' is ═C(H)—P(O)(OH); and the double bond between the C5′ carbon and R⁵′ is in the E orientation.

A vinyl phosphonate of the instant disclosure may be attached to either the antisense or the sense strand of a dsRNA of the disclosure. In certain preferred embodiments, a vinyl phosphonate of the instant disclosure is attached to the antisense strand of a dsRNA, optionally at the 5′ end of the antisense strand of the dsRNA.

Vinyl phosphate modifications are also contemplated for the compositions and methods of the instant disclosure. An exemplary vinyl phosphate structure is:

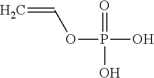

Another exemplary vinyl phosphate structure includes the preceding structure, where R⁵′ is ═C(H)-OP(O)(OH)2 and the double bond between the C5′ carbon and R⁵′ is in the E or Z orientation (e.g., E orientation).
i. Thermally Destabilizing Modifications

In certain embodiments, a dsRNA molecule can be optimized for RNA interference by incorporating thermally destabilizing modifications in the seed region of the antisense strand (i.e., at positions 2-9 of the 5′-end of the antisense strand) to reduce or inhibit off-target gene silencing. It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, or preferably positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s). In some embodiments, the thermally destabilizing modification of the duplex is located at position 2, 3, 4, 5 or 9 from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA; and 2′-5′-linked ribonucleotides (“3′-RNA”)).

Exemplified abasic modifications include, but are not limited to the following:

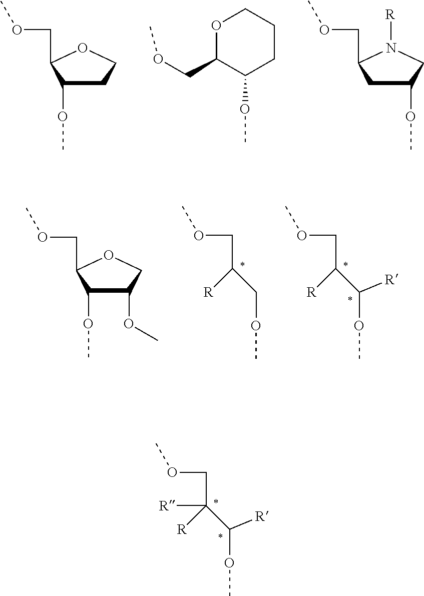

### Wherein R═H, Me, Et or OMe; R′═H, Me, Et or OMe; R″═H, Me, Et or OMe

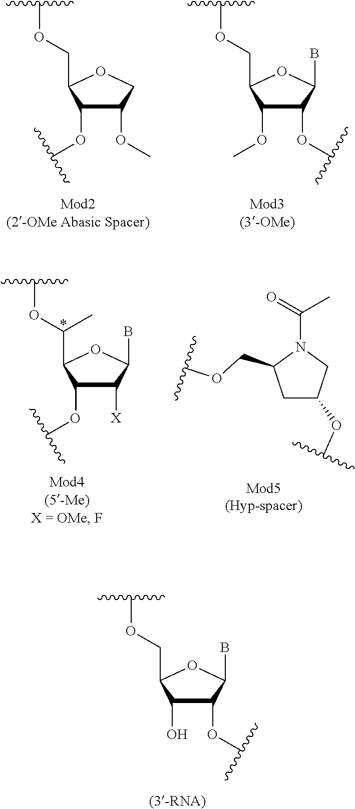

wherein B is a modified or unmodified nucleobase.

Exemplified sugar modifications include, but are not limited to the following:

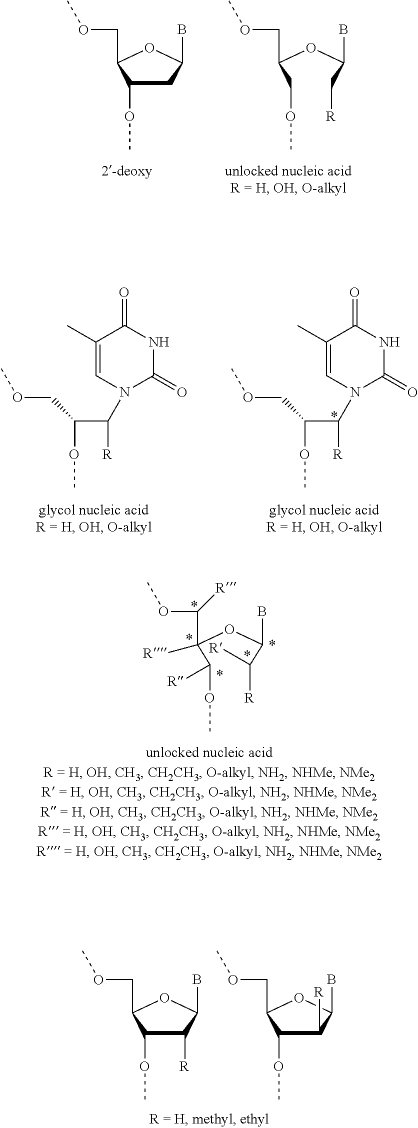

wherein B is a modified or unmodified nucleobase.

In some embodiments the thermally destabilizing modification of the duplex is selected from the group consisting of:

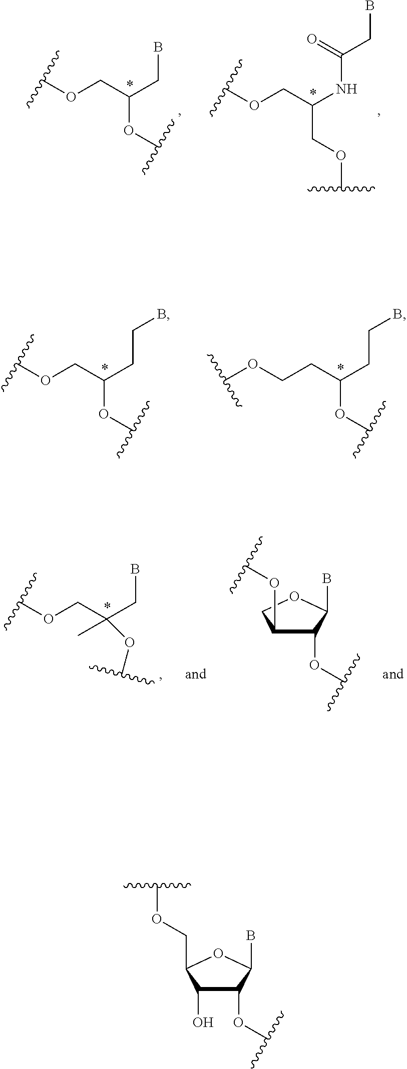

wherein B is a modified or unmodified nucleobase and the asterisk on each structure represents either R, S or racemic.

In some embodiments the thermally destabilizing modification of the duplex is selected from the group consisting of:

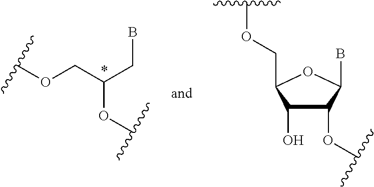

wherein B is a modified or unmodified nucleobase and the asterisk represents either R, S or racemic (e.g. S).

The term “acyclic nucleotide” refers to any nucleotide having an acyclic ribose sugar, for example, where any of bonds between the ribose carbons (e.g., C1′-C2′, C2′-C3′, C3′-C4′, C4′-04′, or C1′-04′) is absent or at least one of ribose carbons or oxygen (e.g., C1′, C2′, C3′, C4′ or 04′) are independently or in combination absent from the nucleotide. In some embodiments, acyclic nucleotide is

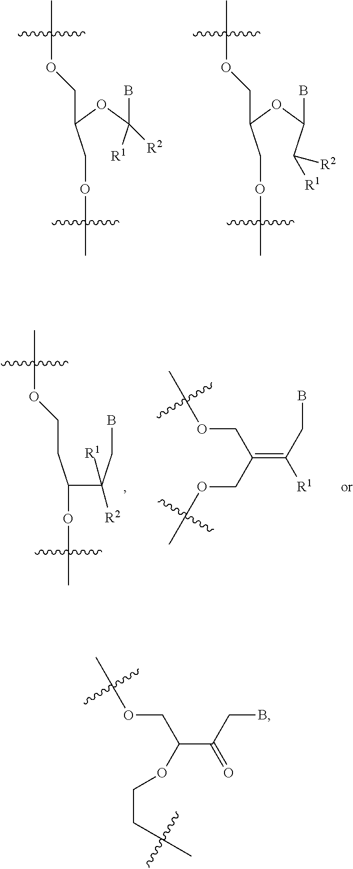

wherein B is a modified or unmodified nucleobase, R′ and R² independently are H, halogen, OR3, or alkyl; and R³ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

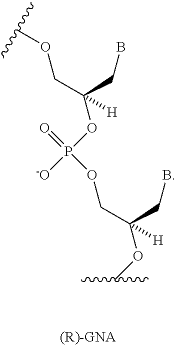

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W-C H-bonding to complementary base on the target mRNA, such as:

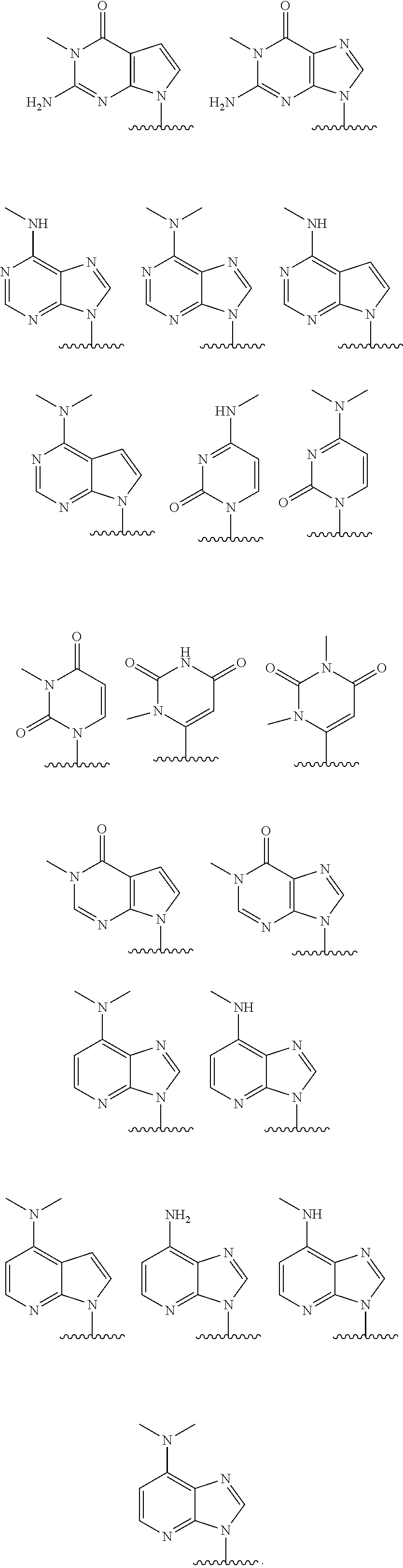

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

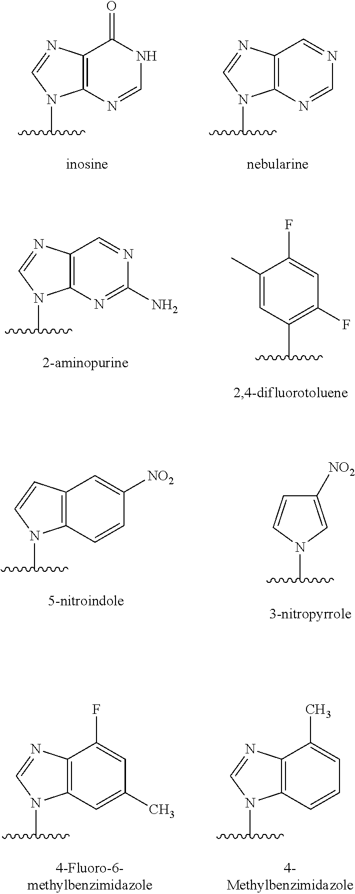

In some embodiments, the thermally destabilizing modification of the duplex in the seed region of the antisense strand includes one or more α-nucleotide complementary to the base on the target mRNA, such as:

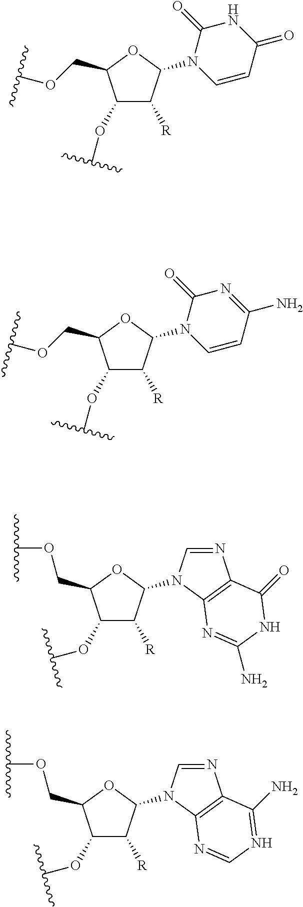

wherein R is H, OH, OCH₃, F. NH₂, NHMe, NMe2 or O-alkyl.

Exemplary phosphate modifications known to decrease the thermal stability of dsRNA duplexes compared to natural phosphodiester linkages are:

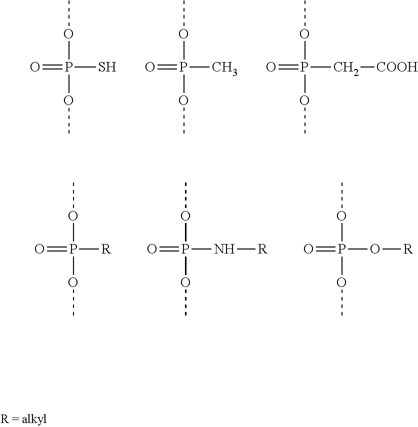

The alkyl for the R group can be a C₁-C₆alkyl. Specific alkyls for the R group include, but are not limited to methyl, ethyl, propyl, isopropyl, butyl, pentyl and hexyl.

As the skilled artisan will recognize, in view of the functional role of nucleobases is defining specificity of an RNAi agent of the disclosure, while nucleobase modifications can be performed in the various manners as described herein, e.g., to introduce destabilizing modifications into an RNAi agent of the disclosure, e.g., for purpose of enhancing on-target effect relative to off-target effect, the range of modifications available and, in general, present upon RNAi agents of the disclosure tends to be much greater for non-nucleobase modifications, e.g., modifications to sugar groups or phosphate backbones of polyribonucleotides. Such modifications are described in greater detail in other sections of the instant disclosure and are expressly contemplated for RNAi agents of the disclosure, either possessing native nucleobases or modified nucleobases as described above or elsewhere herein.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14, and 16 from the 5′-end. In some other embodiments, the antisense comprises stabilizing modifications at positions 2, 6, 14, and 16 from the 5′-end. In still some other embodiments, the antisense comprises stabilizing modifications at positions 2, 14, and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10, and 11 from the 5′-end. In some other embodiments, the sense strand comprises stabilizing modifications at positions 7, 9, 10, and 11 from the 5′-end. In some embodiments, the sense strand comprises stabilizing modifications at positions opposite or complimentary to positions 11, 12, and 15 of the antisense strand, counting from the 5′-end of the antisense strand. In some other embodiments, the sense strand comprises stabilizing modifications at positions opposite or complimentary to positions 11, 12, 13, and 15 of the antisense strand, counting from the 5′-end of the antisense strand. In some embodiments, the sense strand comprises a block of two, three, or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to, 2′-fluoro modifications. Other thermally stabilizing modifications include, but are not limited to, LNA.

In some embodiments, the dsRNA of the disclosure comprises at least four (e.g., four, five, six, seven, eight, nine, ten, or more) 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten, or more) 2′-fluoro nucleotides. Without limitations, a 2′-fluoro modification in the antisense strand can be present at any positions. In some embodiments, the antisense comprises 2′-fluoro nucleotides at positions 2, 6, 8, 9, 14, and 16 from the 5′-end. In some other embodiments, the antisense comprises 2′-fluoro nucleotides at positions 2, 6, 14, and 16 from the 5′-end. In still some other embodiments, the antisense comprises 2′-fluoro nucleotides at positions 2, 14, and 16 from the 5′-end.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) 2′-fluoro nucleotides. Without limitations, a 2′-fluoro modification in the sense strand can be present at any positions. In some embodiments, the antisense comprises 2′-fluoro nucleotides at positions 7, 10, and 11 from the 5′-end. In some other embodiments, the sense strand comprises 2′-fluoro nucleotides at positions 7, 9, 10, and 11 from the 5′-end. In some embodiments, the sense strand comprises 2′-fluoro nucleotides at positions opposite or complimentary to positions 11, 12, and 15 of the antisense strand, counting from the 5′-end of the antisense strand. In some other embodiments, the sense strand comprises 2′-fluoro nucleotides at positions opposite or complimentary to positions 11, 12, 13, and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to the thermally destabilizing modification of the duplex in the antisense strand.

In some embodiments, the dsRNA molecule of the disclosure comprises a 21 nucleotides (nt) sense strand and a 23 nucleotides (nt) antisense, wherein the antisense strand contains at least one thermally destabilizing nucleotide, where the at least one thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; and (vii) the dsRNA comprises a blunt end at 5′-end of the antisense strand. Preferably, the 2 nt overhang is at the 3′-end of the antisense.

In some embodiments, the dsRNA molecule of the disclosure comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand). For example, the thermally destabilizing nucleotide occurs between positions opposite or complimentary to positions 14-17 of the 5′-end of the sense strand, and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2. 3, 4, or 5 phosphorothioate internucleotide linkages; (iii) the sense strand is conjugated with a ligand; (iv) the sense strand comprises 2, 3. 4, or 5 2′-fluoro modifications; (v) the sense strand comprises 1, 2. 3, 4, or 5 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA molecule of the disclosure comprises a sense and antisense strands, wherein said dsRNA molecule comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′ end, wherein the 3′ end of said sense strand and the 5′ end of said antisense strand form a blunt end and said antisense strand is 1˜4 nucleotides longer at its 3′ end than the sense strand, wherein the duplex region which is at least 25 nucleotides in length, and said antisense strand is sufficiently complementary to a target mRNA along at least 19 nt of said antisense strand length to reduce target gene expression when said dsRNA molecule is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2. 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA has a duplex region of 12-29 nucleotide pairs in length.

In some embodiments, every nucleotide in the sense strand and antisense strand of the dsRNA molecule may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy. 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxy-2′-fluoro nucleotide, 2′-O-N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand

In some embodiments, the dsRNA molecule of the disclosure comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . .” “AABBAABBAABB . . . .” “AABAABAABAAB . . . .” “AAABAAABAAAB . . . .” “AAABBBAAABBB . . . .” or “ABCABCABCABC . . . ,” etc. The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . “, “ACACAC . . . ” “BDBDBD . . . ” or “CDCDCD . . . .” etc.

In some embodiments, the dsRNA molecule of the disclosure comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In one particular example, the alternating motif in the sense strand is “ABABAB” from 5′-3′ of the strand, where each A is an unmodified ribonucleotide and each B is a 2′-Omethyl modified nucleotide.

In one particular example, the alternating motif in the sense strand is “ABABAB” from 5′-3′ of the strand, where each A is an 2′-deoxy-2′-fluoro modified nucleotide and each B is a 2′-Omethyl modified nucleotide.

In another particular example, the alternating motif in the antisense strand is “BABABA” from 3′-5′ of the strand, where each A is a 2′-deoxy-2′-fluoro modified nucleotide and each B is a 2′-Omethyl modified nucleotide.

In one particular example, the alternating motif in the sense strand is “ABABAB” from 5′-3′ of the strand and the alternating motif in the antisense strand is “BABABA” from 3′-5′ of the strand, where each A is an unmodified ribonucleotide and each B is a 2′-Omethyl modified nucleotide.

In one particular example, the alternating motif in the sense strand is “ABABAB” sfrom 5′-3′ of the strand and the alternating motif in the antisense strand is “BABABA” from 3′-5′ of the strand, where each A is a 2′-deoxy-2′-fluoro modified nucleotide and each B is a 2′-Omethyl modified nucleotide.

The dsRNA molecule of the disclosure may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13, or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11, or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9, or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7, or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5, or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3, or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the disclosure further comprises one or more phosphorothioate or methylphosphonate internucleotide linkage modification within 1-10 of the termini position(s) of the sense or antisense strand. For example, at least 2, 3, 4, 5, 6, 7, 8, 9, or 10 nucleotides may be linked through phosphorothioate or methylphosphonate internucleotide linkage at one end or both ends of the sense or antisense strand.

In some embodiments, the dsRNA molecule of the disclosure further comprises one or more phosphorothioate or methylphosphonate internucleotide linkage modification within 1-10 of the internal region of the duplex of each of the sense or antisense strand. For example, at least 2, 3, 4, 5, 6, 7, 8, 9, or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the disclosure further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA molecule of the disclosure further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, compound of the disclosure comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the disclosure comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the disclosure comprises a 5′-block is an Sp block wherein each sugar moiety comprises a 2′-F modification. In some embodiments, a 5′-block is an Sp block wherein each of internucleotidic linkage is a modified internucleotidic linkage and each sugar moiety comprises a 2′-F modification. In some embodiments, a 5′-block is an Sp block wherein each of internucleotidic linkage is a phosphorothioate linkage and each sugar moiety comprises a 2′-F modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-F modification. In some embodiments, a 3′-block is an Sp block wherein each of internucleotidic linkage is a modified internucleotidic linkage and each sugar moiety comprises a 2′-F modification. In some embodiments, a 3′-block is an Sp block wherein each of internucleotidic linkage is a phosphorothioate linkage and each sugar moiety comprises a 2′-F modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the disclosure comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments. A is followed by Sp. In some embodiments. A is followed by Rp. In some embodiments. A is followed by natural phosphate linkage (PO). In some embodiments. U is followed by Sp. In some embodiments. U is followed by Rp. In some embodiments. U is followed by natural phosphate linkage (PO). In some embodiments. C is followed by Sp. In some embodiments. C is followed by Rp. In some embodiments. C is followed by natural phosphate linkage (PO). In some embodiments. G is followed by Sp. In some embodiments, G is followed by Rp. In some embodiments. G is followed by natural phosphate linkage (PO). In some embodiments. C and U are followed by Sp. In some embodiments. C and U are followed by Rp. In some embodiments. C and U are followed by natural phosphate linkage (PO). In some embodiments. A and G are followed by Sp. In some embodiments. A and G are followed by Rp.

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3. 4, 5 or 6 2′-fluoro modifications; (ii) the antisense comprises 3, 4 or 5 phosphorothioate internucleotide linkages; (iii) the sense strand is conjugated with a ligand; (iv) the sense strand comprises 2, 3. 4 or 5 2′-fluoro modifications; (v) the sense strand comprises 1, 2. 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3. 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2. 3, 4 or 5 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand. In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3, 4 or 5 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In some embodiments, the dsRNA molecule of the disclosure comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA molecule of the disclosure comprises at least one of the first 1, 2, 3, 4, or 5 base pairs within the duplex regions from the 5′-end of the antisense strand can be chosen independently from the group of: A:U. G:U. I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

It was found that introducing 4′-modified or 5′-modified nucleotide to the 3′-end of a phosphodiester (PO), phosphorothioate (PS), or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of single stranded or double stranded siRNA. For instance, a 5′-alkylated nucleoside may be introduced at the 3′-end of a dinucleotide at any position of single stranded or double stranded siRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, 4′-modified nucleoside is introduced at the 3′-end of a dinucleotide at any position of single stranded or double stranded siRNA. For instance, a 4′-alkylated nucleoside may be introduced at the 3′-end of a dinucleotide at any position of single stranded or double stranded siRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, 5′-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or Sisomer. In some embodiments, 4′-alkylated nucleoside is introduced at any position on the sense strand or antisense strand of a dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, 4′-O-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or Sisomer.

In some embodiments, the dsRNA molecule of the disclosure can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA molecule of the disclosure can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC.

Various publications describe multimeric siRNA which can all be used with the dsRNA of the disclosure. Such publications include WO2007/091269, U.S. Pat. No. 7,858,769, WO2010/141511. WO2007/117686, WO2009/014887, and WO2011/031520 which are hereby incorporated by their entirely.

As described in more detail below, the RNAi agent that contains conjugations of one or more carbohydrate moieties to an RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds. The ligand may be attached to the polynucleotide via a carrier. The carriers include (i) at least one “backbone attachment point.” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be. e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

In certain specific embodiments, the RNAi agent for use in the methods of the disclosure is an agent selected from the group of agents listed in any one of Tables 2, 3, 5, 6, 8, 9, 10A, 10B, 10C, 10D, 11, and 12. These agents may further comprise a ligand.

IV. iRNAs Conjugated to Ligands

Another modification of the RNA of an iRNA of the invention involves chemically linking to the iRNA one or more ligands, moieties or conjugates that enhance the activity, cellular distribution or cellular uptake of the iRNA, e.g., into a cell. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thiocther, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J. 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting or lifetime of an iRNA agent into which it is incorporated. In some embodiments, a ligand provides an enhanced affinity for a selected target, e.g., molecule, cell or cell type, compartment, e.g., a cellular or organ compartment, tissue, organ or region of the body, as, e.g., compared to a species absent such a ligand. Typical ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include; polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an a helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone. 1.3-Bis-O(hexadecyl)glycerol, geranyloxyhexyl group, hexadecylglycerol, borneol, menthol, 1.3-propanediol, heptadecyl group, palmitic acid, myristic acid, O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. Such a lipid or lipid-based molecule can typically bind a serum protein, such as human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid-based ligand can be used to modulate, e.g., control (e.g., inhibit) the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid-based ligand binds HSA. For example, the ligand can bind HSA with a sufficient affinity such that distribution of the conjugate to a non-kidney tissue is enhanced. However, the affinity is typically not so strong that the HSA-ligand binding cannot be reversed.

In certain embodiments, the lipid-based ligand binds HSA weakly or not at all, such that distribution of the conjugate to the kidney is enhanced. Other moieties that target to kidney cells can also be used in place of or in addition to the lipid-based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

### B. Cell Permeation Agents

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is typically an α-helical agent and can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 104). An RFGF analogue (e.g., amino acid sequence AALLPVLLAAP (SEQ ID NO: 105)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 106)) and the Drosophila Antennapedia protein (RQIKIWFQNRRMKWKK (SEQ ID NO: 107)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Typically, the peptide or peptidomimetic tethered to a dsRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

An RGD peptide moiety can be used to target a particular cell type, e.g., a tumor cell, such as an endothelial tumor cell or a breast cancer tumor cell (Zitzmann et al., Cancer Res., 62:5139-43, 2002). An RGD peptide can facilitate targeting of an dsRNA agent to tumors of a variety of other tissues, including the lung, kidney, spleen, or liver (Aoki et al., Cancer Gene Therapy 8:783-787, 2001). Typically, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing avß; (Haubner et al., Jour. Nucl. Med., 42:326-336, 2001).

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simconi et al., Nucl. Acids Res. 31:2717-2724, 2003).

### C. Carbohydrate Conjugates

In some embodiments of the compositions and methods of the invention, an iRNA further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and tri-saccharides include sugars having two or three monosaccharide units (e.g., C5, C6, C7, or C8).

In certain embodiments, a carbohydrate conjugate comprises a monosaccharide.

In certain embodiments, the monosaccharide is an N-acetylgalactosamine (GalNAc). GalNAc conjugates, which comprise one or more N-acetylgalactosamine (GalNAc) derivatives, are described, for example, in U.S. Pat. No. 8,106,022, the entire content of which is hereby incorporated herein by reference. In some embodiments, the GalNAc conjugate serves as a ligand that targets the iRNA to particular cells. In some embodiments, the GalNAc conjugate targets the iRNA to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the iRNA agent (e.g., to the 3′ end of the sense strand) via a linker, e.g., a linker as described herein. In some embodiments the GalNAc conjugate is conjugated to the 5′ end of the sense strand. In some embodiments, the GalNAc conjugate is conjugated to the iRNA agent (e.g., to the 5′ end of the sense strand) via a linker, e.g., a linker as described herein.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker. In other embodiments of the invention, the GalNAc or GalNAc derivative is attached to an iRNA agent of the invention via a tetravalent linker.

In certain embodiments, the double stranded RNAi agents of the invention comprise one GalNAc or GalNAc derivative attached to the iRNA agent. In certain embodiments, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, the GalNAc conjugate is

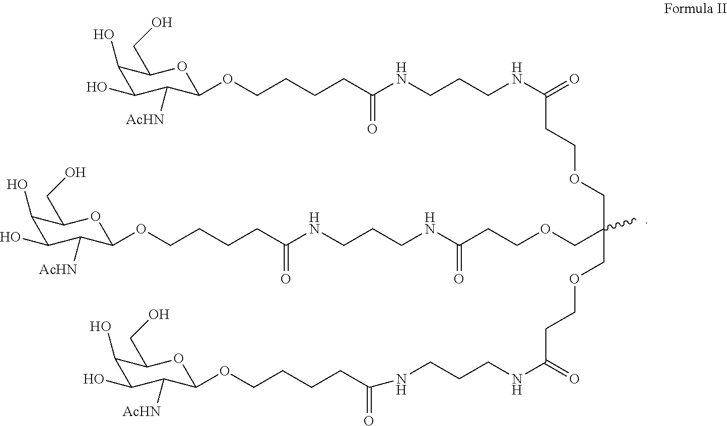 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent is attached to the carbohydrate conjugate via a linker as shown in the following schematic, wherein X is O or S

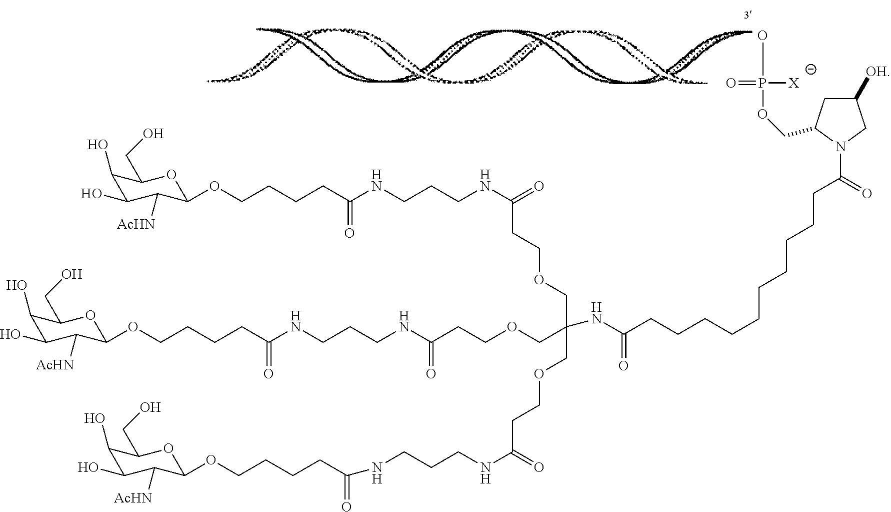

In some embodiments, the RNAi agent is conjugated to L96 as defined in Table 1 and shown below:

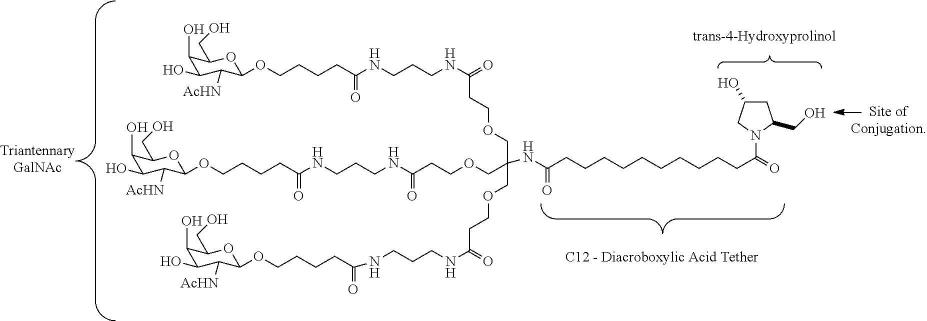

In certain embodiments, a carbohydrate conjugate for use in the compositions and methods of the invention is selected from the group consisting of:

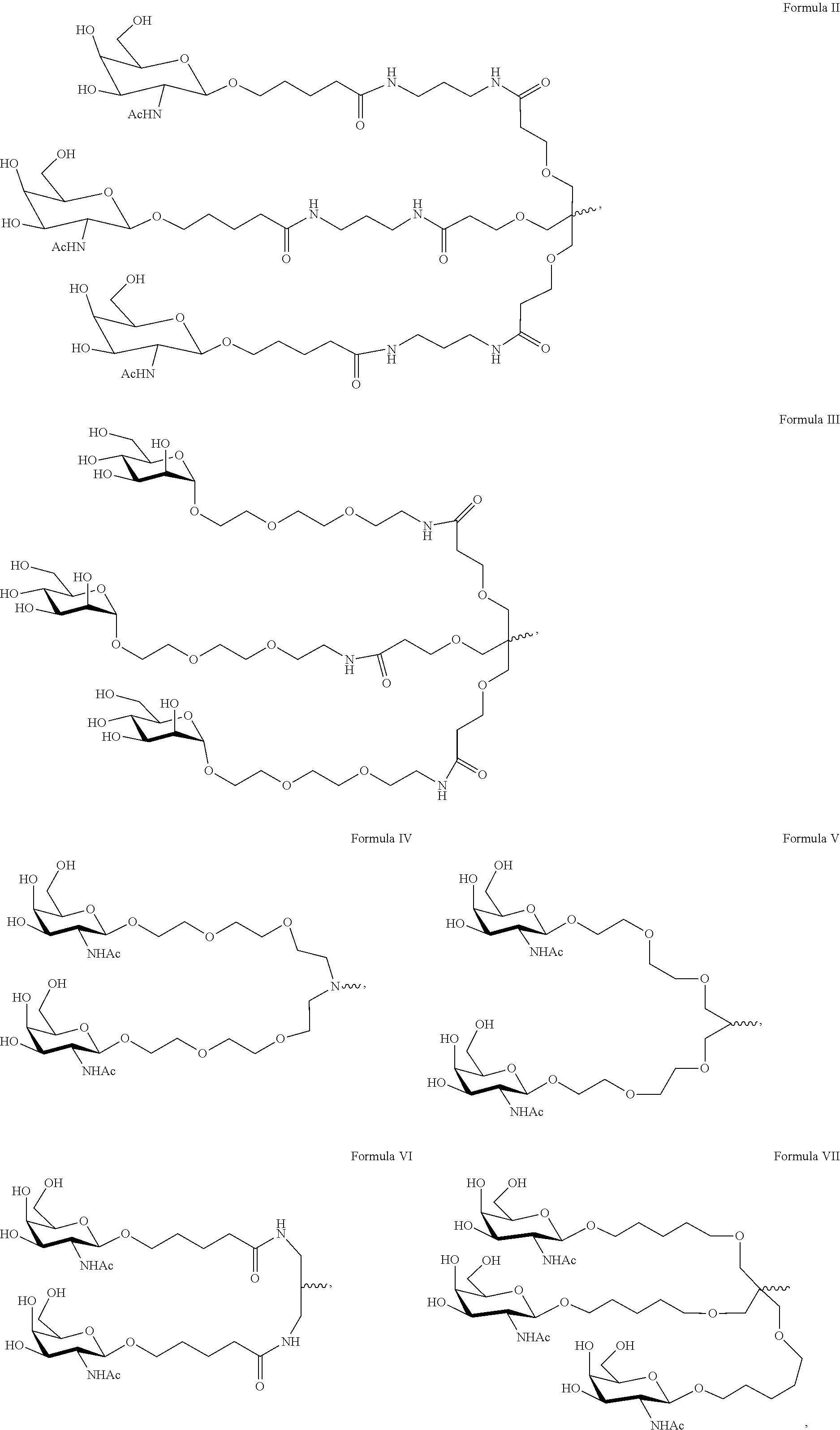

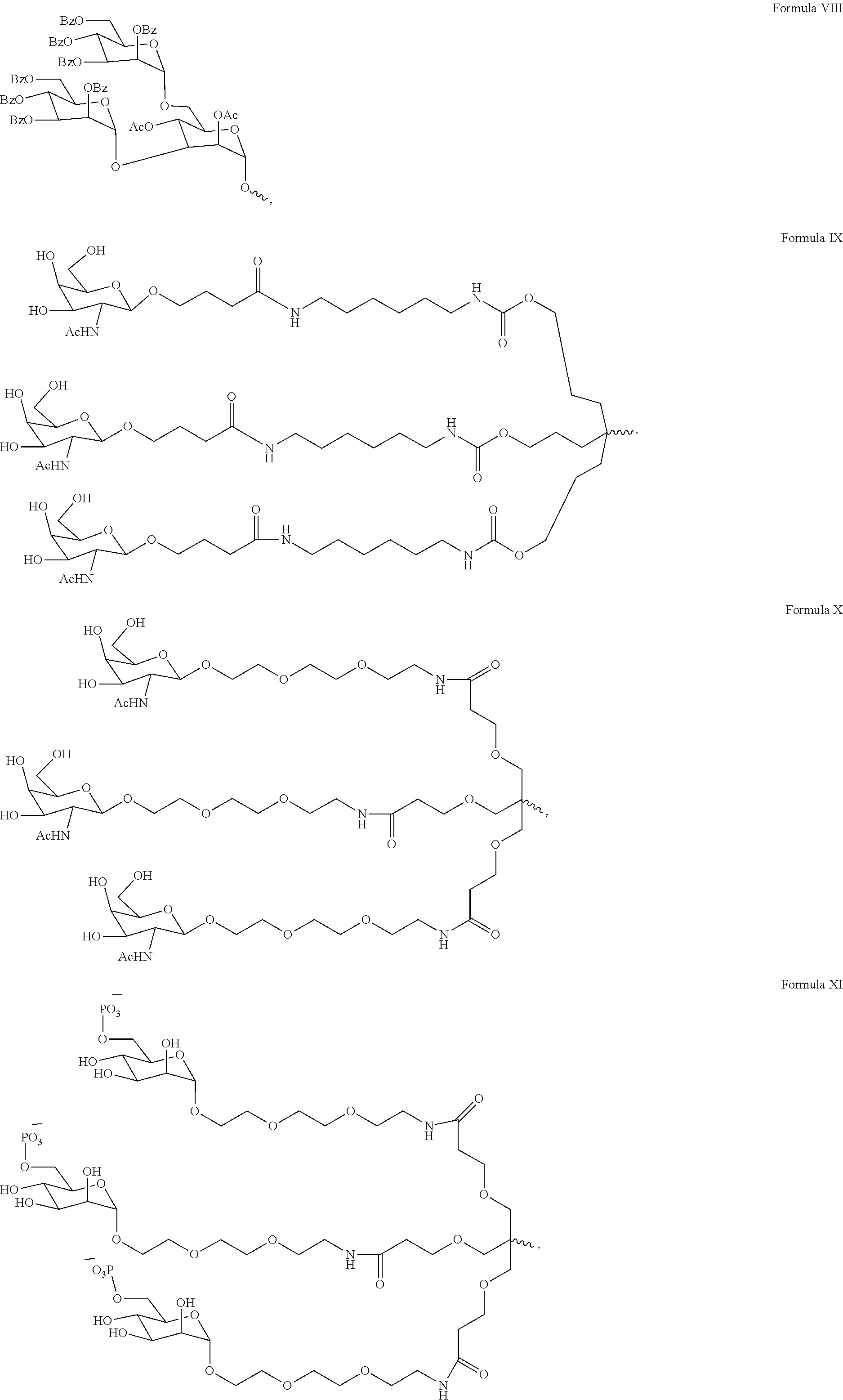

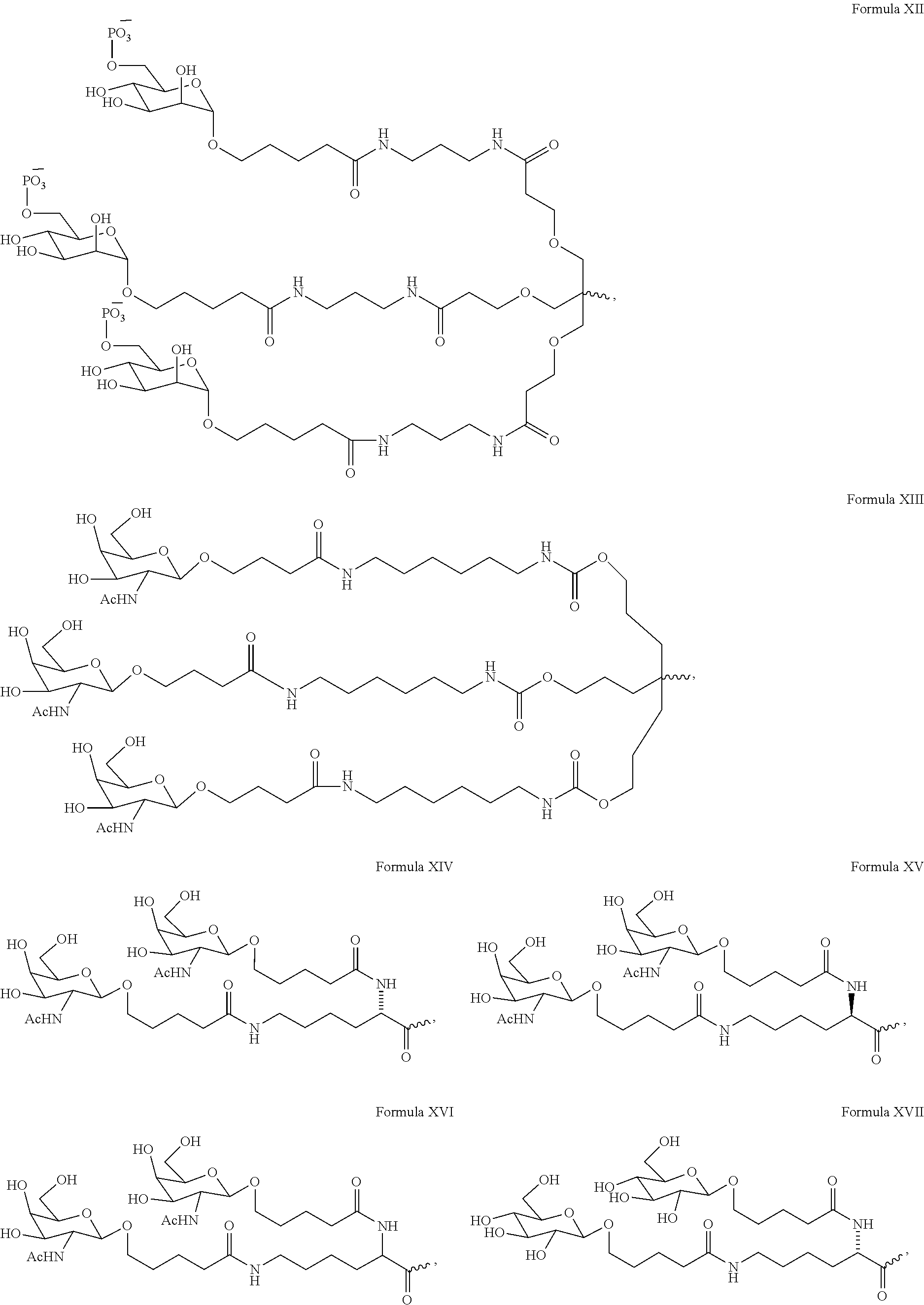

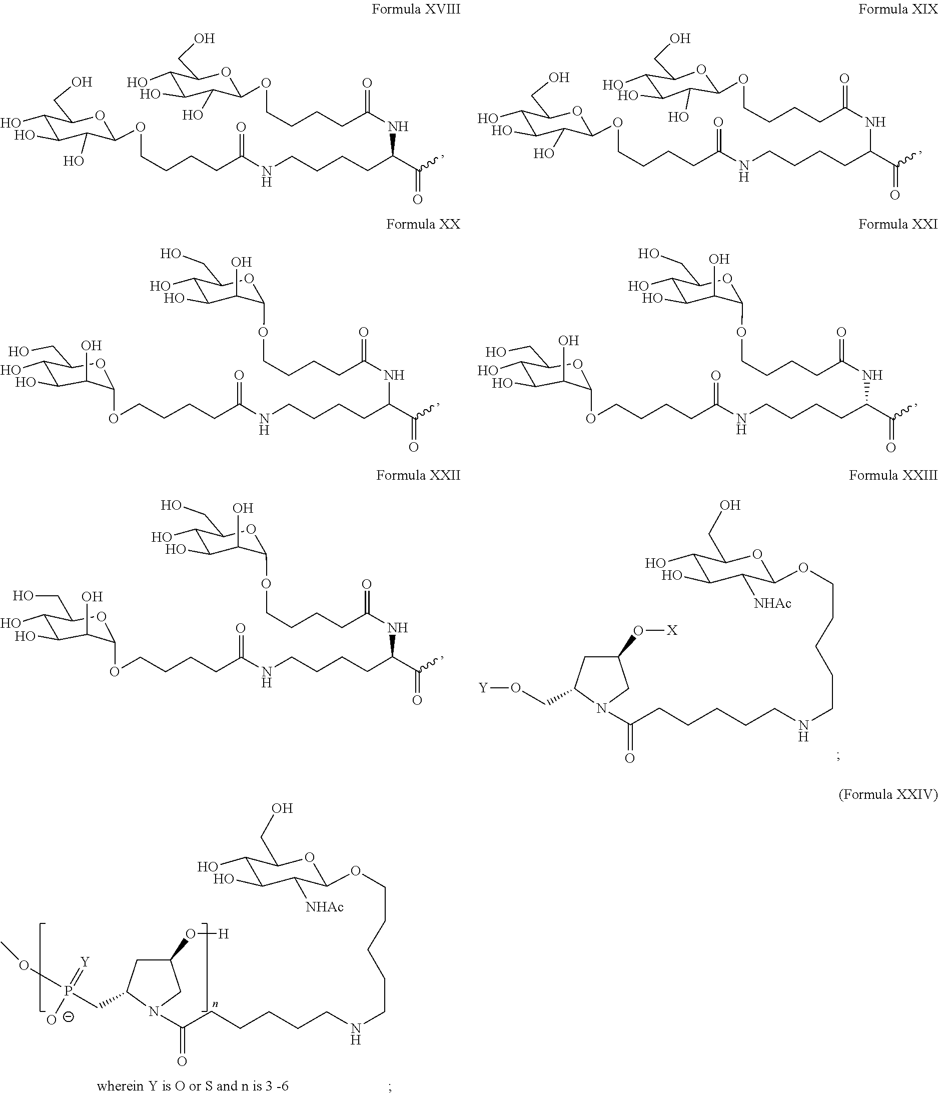

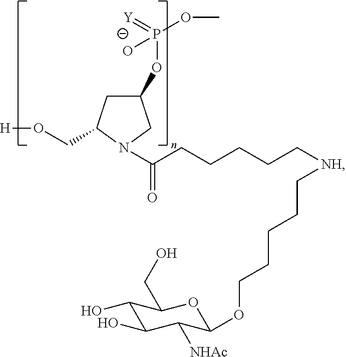

wherein Y is O or S and n is 3-6 (Formula XXV);

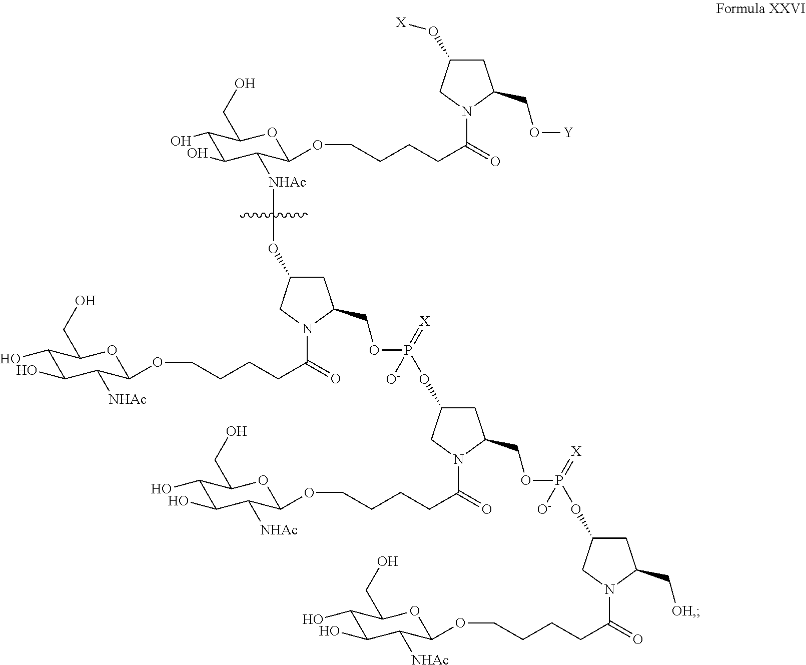

wherein X is O or S (Formula XXVII);

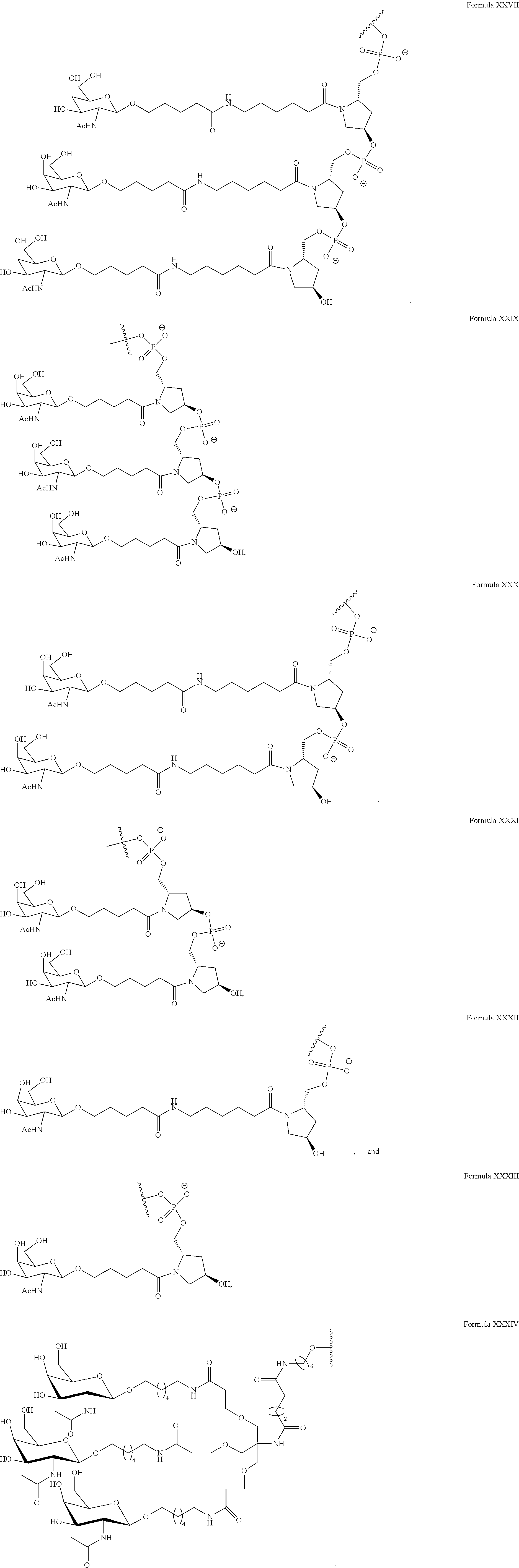

In certain embodiments, a carbohydrate conjugate for use in the compositions and methods of the invention is a monosaccharide. In certain embodiments, the monosaccharide is an N-acetylgalactosamine, such as

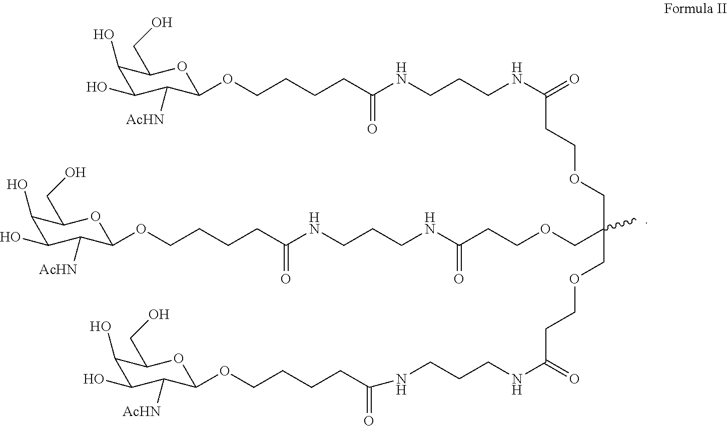 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to.

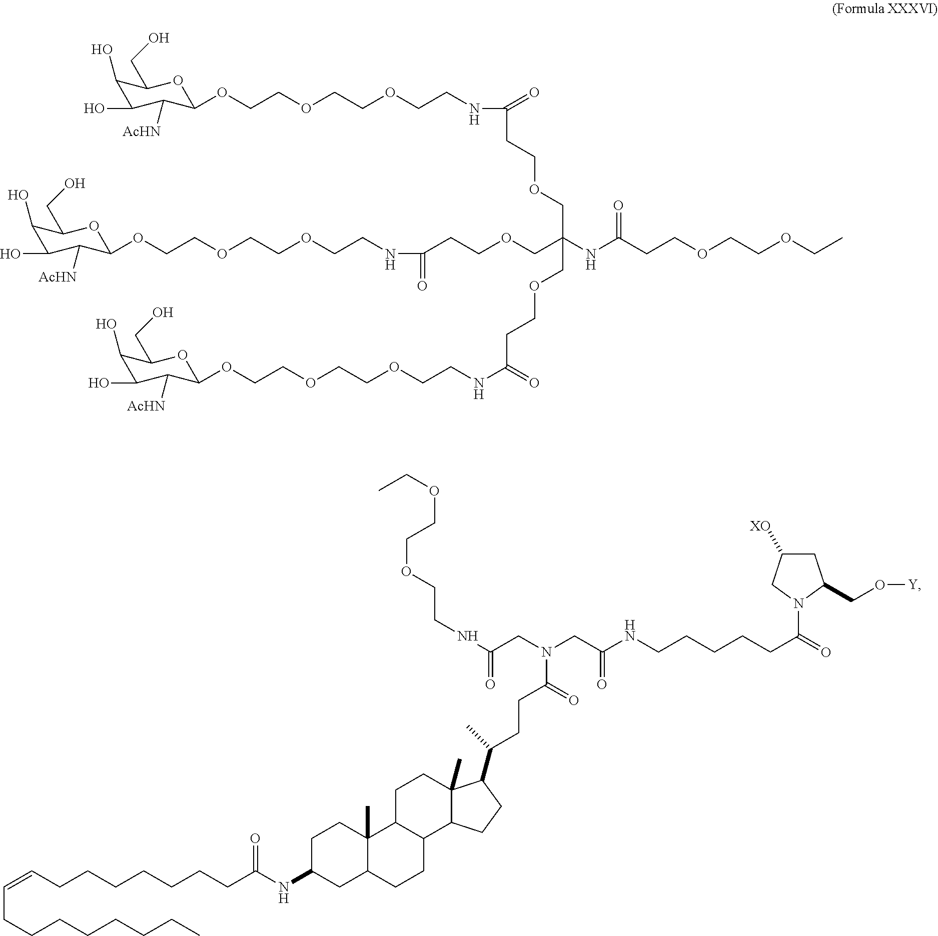

when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below:

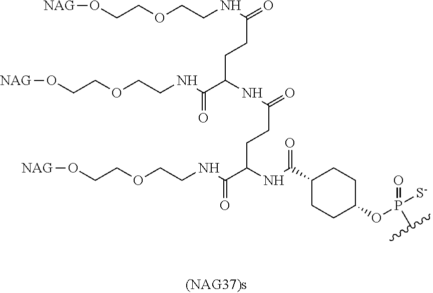

In certain embodiments, the RNAi agents of the disclosure may include GalNAc ligands, even if such GalNAc ligands are currently projected to be of limited value for the preferred intrathecal/CNS delivery route(s) of the instant disclosure.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker.

In one embodiment, the double stranded RNAi agents of the invention comprise one or more GalNAc or GalNAc derivative attached to the iRNA agent. The GalNAc may be attached to any nucleotide via a linker on the sense strand or antsisense strand. The GalNac may be attached to the 5′-end of the sense strand, the 3′ end of the sense strand, the 5′-end of the antisense strand, or the 3′-end of the antisense strand. In one embodiment, the GalNAc is attached to the 3′ end of the sense strand, e.g., via a trivalent linker.

In other embodiments, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of linkers, e.g., monovalent linkers.

In some embodiments, for example, when the two strands of an iRNA agent of the invention is part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O. S. S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In certain embodiments, the linker is between about 1-24 atoms, 2-24, 3-24, 4-24, 5-24, 6-24, 6-18, 7-18, 8-18 atoms, 7-17, 8-17, 6-16, 7-16, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4. 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group.” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### ii Phosphate-Based Cleavable Linking Groups

In certain embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)—O—, —O—P(S)(ORk)—O—, —O—P(S)(SRk)—O—, —S—P(O)(ORk)—O—, —O—P(O)(ORk)—S—, —S—P(O)(ORk)—S—, —O—P(S)(ORk)—S—, —S—P(S)(ORk)—O—, —O—P(O)(Rk)—O—, —O—P(S)(Rk)—O—, —S—P(O)(Rk)—O—, —S—P(S)(Rk)—O—, —S—P(O)(Rk)—S—, —O—P(S)(Rk)—S—. Preferred embodiments are —O—P(O)(OH)—O—, —O—P(S)(OH)—O—, —O—preferred embodiment is —O—P(O)(OH)—O—. These candidates can be evaluated using methods analogous to those described above.

iii. Acid Cleavable Linking Groups

In certain embodiments, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Cleavable Linking Groups

In certain embodiments, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

v. Peptide-Based Cleavable Linking Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula—NHCHRAC(O)NHCHRBC(O)—, where RA and RB are the R groups of the two adjacent amino acids. These candidates can be evaluated using methods analogous to those described above.

In some embodiments, an iRNA of the invention is conjugated to a carbohydrate through a linker. Non-limiting examples of iRNA carbohydrate conjugates with linkers of the compositions and methods of the invention include, but are not limited to.

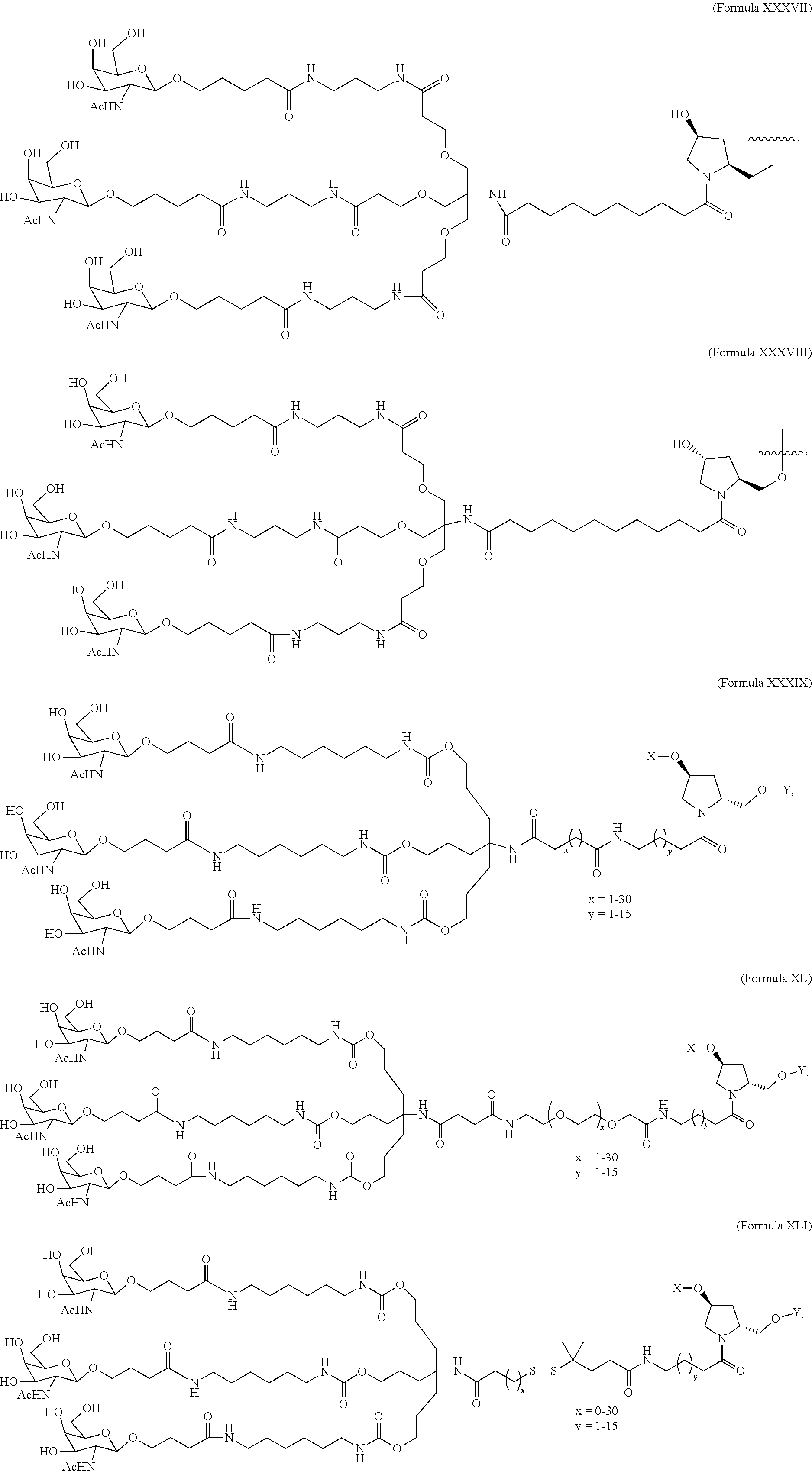

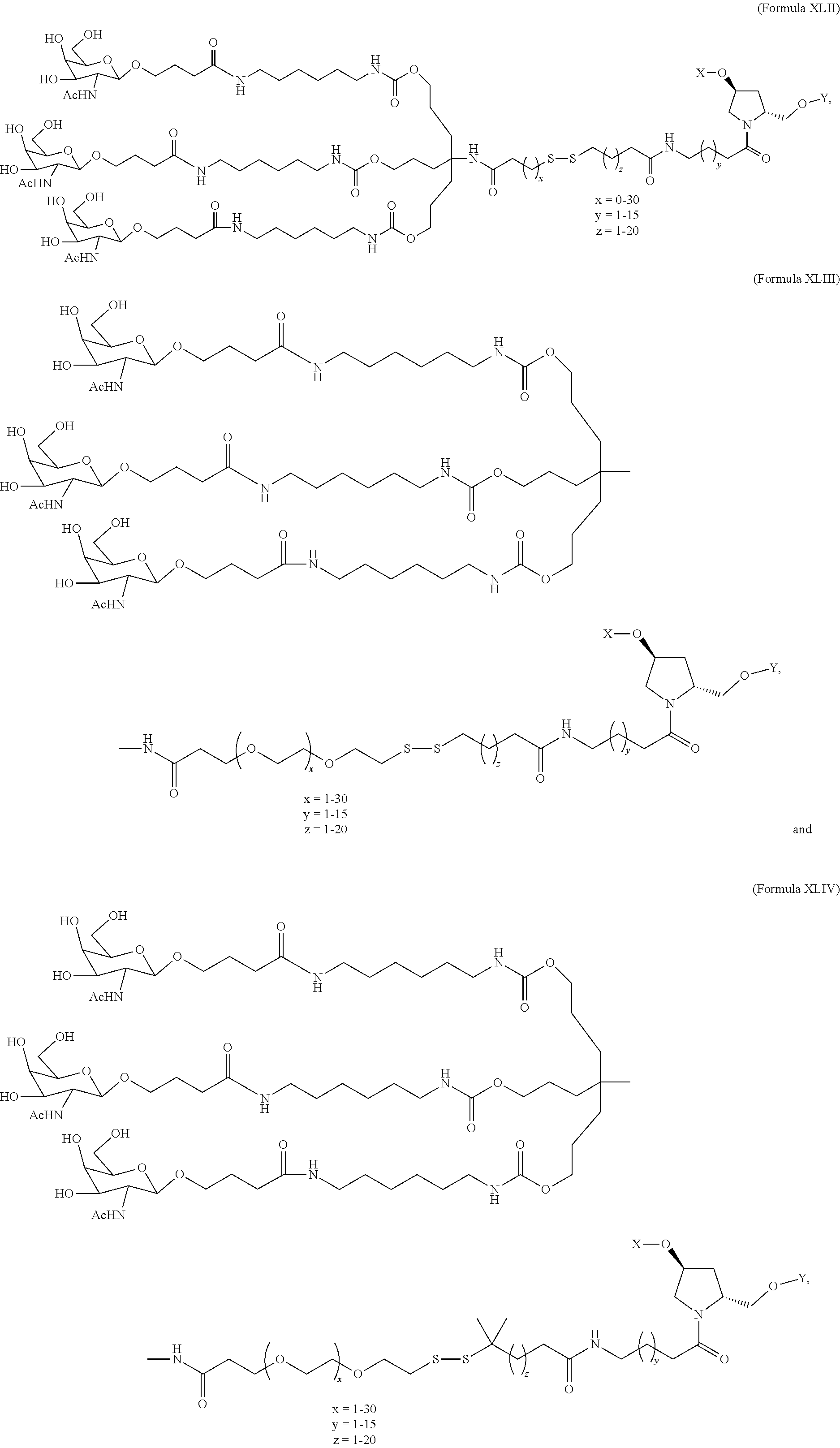

when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In certain embodiments, a deRNA of the invention is conjugated to a bivalent or trivalent branched linker selected from the group of structures shown in any of formula (XLV)-(XLVI):

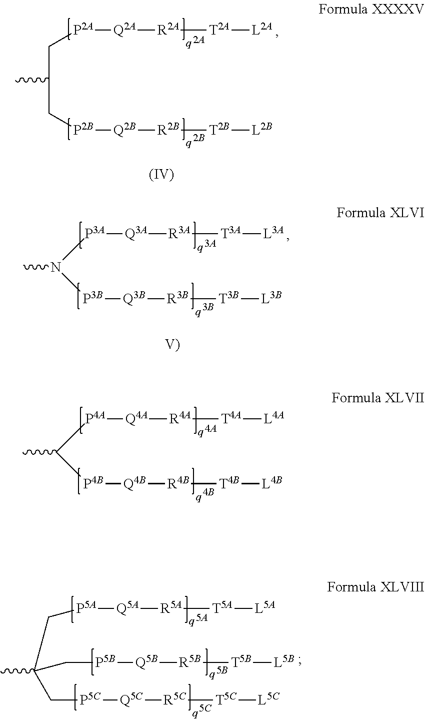

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(RN), C(R′)═C(R″), C═C or C(O);
R^{2A}, R^{2B}, R^{3A}, R^{3B}, R^{4A}, R^{4B}, R^{5A}, R^{5B}, R^{5C} are each independently for each occurrence absent,
   NH, O, S, CH₂, C(O)O, C(O)NH, NHCH(R^{a})C(O), —C(O)—CH(Ra)—NH—, CO, CH═N—O,

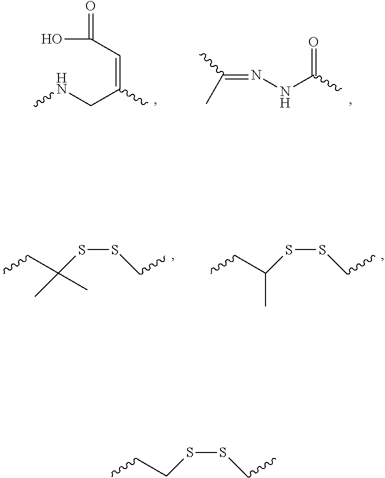

or heterocyclyl;

L^{2A}, L^{2B}, L^{3A}, L^{3B}, L^{4A}, L^{4B}, L^{5A}, L⁵B and L⁵° C. represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

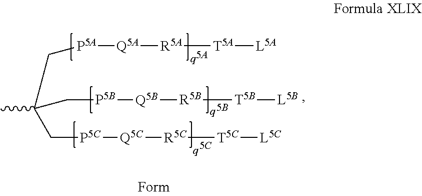

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. Patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNA agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA: RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### V. Delivery of an RNAi Agent of the Disclosure

The delivery of an RNAi agent of the disclosure to a cell e.g., a cell within a subject, such as a human subject (e.g., a subject in need thereof, such as a subject having a C9orf72-associated disorder, e.g., C9orf72-associated disease, can be achieved in a number of different ways. For example, delivery may be performed by contacting a cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with an RNAi agent of the disclosure (see e.g., Akhtar S. and Julian RL., (1992) Trends Cell. Biol. 2(5): 139-144 and WO94/02595, which are incorporated herein by reference in their entireties). For in vivo delivery, factors to consider in order to deliver an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, intraocular delivery of a VEGF dsRNA by intravitreal injection in cynomolgus monkeys (Tolentino, M J. et al., (2004) Retina 24:132-138) and subretinal injections in mice (Reich, S J. et al. (2003) Mol. Vis. 9:210-216) were both shown to prevent neovascularization in an experimental model of age-related macular degeneration. In addition, direct intratumoral injection of a dsRNA in mice reduces tumor volume (Pille. J. et al. (2005) Mol. Ther. 11:267-274) and can prolong survival of tumor-bearing mice (Kim. W J. et al., (2006) Mol. Ther. 14:343-350; Li. S. et al., (2007) Mol. Ther. 15:515-523). RNA interference has also shown success with local delivery to the CNS by direct injection (Dorn. G. et al., (2004) Nucleic Acids 32:c49; Tan. PH. et al. (2005) Gene Ther. 12:59-66; Makimura, H. et al. (2002) BMC Neurosci. 3:18; Shishkina. GT., et al. (2004) Neuroscience 129:521-528; Thakker, E R., et al. (2004) Proc. Natl. Acad. Sci. U.S.A. 101:17270-17275; Akancya. Y., et al. (2005) J. Neurophysiol. 93:594-602) and to the lungs by intranasal administration (Howard, K A. et al., (2006) Mol. Ther. 14:476-484; Zhang. X. et al., (2004) J. Biol. Chem. 279:10677-10684; Bitko. V. et al., (2005) Nat. Med. 11:50-55). For administering an RNAi agent systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). RNAi agents can be modified by chemical conjugation to lipophilic groups such as cholesterol to enhance cellular uptake and prevent degradation. For example, an RNAi agent directed against ApoB conjugated to a lipophilic cholesterol moiety was injected systemically into mice and resulted in knockdown of apoB mRNA in both the liver and jejunum (Soutschek. J. et al., (2004) Nature 432:173-178). Conjugation of an RNAi agent to an aptamer has been shown to inhibit tumor growth and mediate tumor regression in a mouse model of prostate cancer (McNamara, J O. et al., (2006) Nat. Biotechnol. 24:1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent, or induced to form a vesicle or micelle (see e.g., Kim SH. et al., (2008) Journal of Controlled Release 129(2): 107-116) that encases an RNAi agent. The formation of vesicles or micelles further prevents degradation of the RNAi agent when administered systemically. Methods for making and administering cationic-RNAi agent complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of RNAi agents include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet ME. et al., (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an RNAi agent forms a complex with cyclodextrin for systemic administration. Methods for administration and pharmaceutical compositions of RNAi agents and cyclodextrins can be found in U.S. Pat. No. 7, 427, 605, which is herein incorporated by reference in its entirety.

Certain aspects of the instant disclosure relate to a method of reducing the expression of a C9orf72 target gene in a cell, comprising contacting said cell with the double-stranded RNAi agent of the disclosure. In one embodiment, the cell is an extraheptic cell, optionally a CNS cell.

Another aspect of the disclosure relates to a method of reducing the expression of a C9orf72 target gene in a subject, comprising administering to the subject the double-stranded RNAi agent of the disclosure.

Another aspect of the disclosure relates to a method of treating a subject having a CNS disorder, comprising administering to the subject a therapeutically effective amount of the double-stranded C9orf72-targeting RNAi agent of the disclosure, thereby treating the subject. Exemplary CNS disorders that can be treated by the method of the disclosure include C9orf72-associated disease.

In one embodiment, the double-stranded RNAi agent is administered intrathecally. By intrathecal administration of the double-stranded RNAi agent, the method can reduce the expression of a C9orf72 target gene in a brain (e.g., striatum) or spine tissue, for instance, cortex, cerebellum, cervical spine, lumbar spine, and thoracic spine.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure. A composition that includes an RNAi agent can be delivered to a subject by a variety of routes. Exemplary routes include: intrathecal, intravenous, topical, rectal, anal, vaginal, nasal, pulmonary, and ocular.

The RNAi agents of the disclosure can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular or intracerebroventricular administration.

The route and site of administration may be chosen to enhance targeting. For example, to target muscle cells, intramuscular injection into the muscles of interest would be a logical choice. Lung cells might be targeted by administering the RNAi agent in aerosol form. The vascular endothelial cells could be targeted by coating a balloon catheter with the RNAi agent and mechanically introducing the RNA.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for intrathecal or intraventricular administration may include sterile aqueous solutions which may also contain buffers, diluents, and other suitable additives.

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the siRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracerebroventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The medication can be provided in measured doses or in a dispenser which delivers a metered dose. Selected modes of delivery are discussed in more detail below.

### A. Intrathecal Administration.

In one embodiment, the double-stranded RNAi agent is delivered by intrathecal injection (i.e., injection into the spinal fluid which bathes the brain and spinal cord tissue). Intrathecal injection of RNAi agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal chord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in WO 2015/116658, which is incorporated by reference in its entirety.

The amount of intrathecally injected RNAi agents may vary from one target gene to another target gene and the appropriate amount that has to be applied may have to be determined individually for each target gene. Typically, this amount ranges from 10 μg to 2 mg, preferably 50 μg to 1500 μg, more preferably 100 μg to 1000 μg.

B. Vector encoded RNAi agents of the Disclosure

RNAi agents targeting the C9orf72 gene can be expressed from transcription units inserted into DNA or RNA vectors (see, e.g., Couture, A, et al., TIG. (1996), 12:5-10; WO 00/22113, WO 00/22114, and U.S. Pat. No. 6,054,299). Expression is preferablysustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of an RNAi agent can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNAi agent expression vectors are generally DNA plasmids or viral vectors. Expression vectors compatible with eukaryotic cells, preferably those compatible with vertebrate cells, can be used to produce recombinant constructs for the expression of an RNAi agent as described herein. Delivery of RNAi agent expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an RNAi agent will generally require regulatory elements, e.g., promoters, enhancers, etc., to ensure the expression of the RNAi agent in target cells. Other aspects to consider for vectors and constructs are known in the art.

### VI. Compositions of the Invention

The present disclosure also includes compositions, including pharmaceutical compositions and formulations which include the RNAi agents of the disclosure.

For example, in one embodiment, the present invention provides compositions comprising two or more, e.g., 2, 3, or 4, dsRNA agents,

In another embodiment, provided herein are pharmaceutical compositions containing an RNAi agent, as described herein, and a pharmaceutically acceptable carrier. The pharmaceutical compositions containing the RNAi agent are useful for treating a disease or disorder associated with the expression or activity of C9orf72. e.g., C9orf72-associated disease.

In some embodiments, the pharmaceutical compositions of the invention are sterile. In another embodiment, the pharmaceutical compositions of the invention are pyrogen free.

Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery. Another example is compositions that are formulated for direct delivery into the CNS, e.g., by intrathecal or intravitreal or intraventricular or intracerebroventricular administration, or by intraroutes of injection, optionally by infusion into the brain (e.g., striatum), such as by continuous pump infusion.

The pharmaceutical compositions of the disclosure may be administered in dosages sufficient to inhibit expression of a C9orf72 gene. In general, a suitable dose of an RNAi agent of the disclosure will be in the range of about 0.001 to about 200.0 milligrams per kilogram body weight of the recipient per day, generally in the range of about 1 to 50 mg per kilogram body weight per day.

A repeat-dose regimen may include administration of a therapeutic amount of an RNAi agent on a regular basis, such as monthly to once every six months. In certain embodiments, the RNAi agent is administered about once per quarter (i.e., about once every three months) to about twice per year.

After an initial treatment regimen (e.g., loading dose), the treatments can be administered on a less frequent basis.

In other embodiments, a single dose of the pharmaceutical compositions can be long lasting, such that subsequent doses are administered at not more than 1, 2, 3, or 4 or more month intervals. In some embodiments of the disclosure, a single dose of the pharmaceutical compositions of the disclosure is administered once per month. In other embodiments of the disclosure, a single dose of the pharmaceutical compositions of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a composition can include a single treatment or a series of treatments.

Advances in mouse genetics have generated a number of mouse models for the study of various human diseases, such as ALS and FTD that would benefit from reduction in the expression of repeat-containing C9orf72. Such models can be used for in vivo testing of RNAi agents, as well as for determining a therapeutically effective dose. Suitable rodent models are known in the art and include, for example, those described in, for example, Cepeda, et al. (ASN Neuro (2010) 2(2):c00033) and Pouladi, et al. (Nat Reviews (2013) 14:708).

The pharmaceutical compositions of the present disclosure can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal, intraventricular, or intracerebroventricular administration.

The RNAi agents can be delivered in a manner to target a particular tissue, such as the CNS (e.g., neuronal, glial or vascular tissue of the brain) or cell type (e.g., neuron).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the RNAi agents featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). RNAi agents featured in the disclosure can be encapsulated within liposomes or can form complexes thereto, in particular to cationic liposomes. Alternatively, RNAi agents can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

### A. RNAi Agent Formulations Comprising Membranous Molecular Assemblies

An RNAi agent for use in the compositions and methods of the disclosure can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the RNAi agent composition. The lipophilic material isolates the aqueous interior from an aqueous exterior, which typically does not include the RNAi agent composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the RNAi agent are delivered into the cell where the RNAi agent can specifically bind to a target RNA and can mediate RNAi. In some cases the liposomes are also specifically targeted, e.g., to direct the RNAi agent to particular cell types.

A liposome containing an RNAi agent can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The RNAi agent preparation is then added to the micelles that include the lipid component. The cationic groups on the lipid interact with the RNAi agent and condense around the RNAi agent to form a liposome. After condensation, the detergent is removed, e.g., by dialysis, to yield a liposomal preparation of RNAi agent.

If necessary a carrier compound that assists in condensation can be added during the condensation reaction, e.g., by controlled addition. For example, the carrier compound can be a polymer other than a nucleic acid (e.g., spermine or spermidine). pH can also adjusted to favor condensation.

Methods for producing stable polynucleotide delivery vehicles, which incorporate a polynucleotide/cationic lipid complex as structural components of the delivery vehicle, are further described in, e.g., WO 96/37194, the entire contents of which are incorporated herein by reference. Liposome formation can also include one or more aspects of exemplary methods described in Felgner. P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging RNAi agent preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid or phosphatidylcholine or cholesterol.

Examples of other methods to introduce liposomes into cells in vitro and in vivo include U.S. Pat. Nos. 5,283,185; 5,171,678; WO 94/00569; WO 93/24640; WO 91/16024; Felgner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{MI}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglioside GMI, galactocerebroside sulfate and phosphatidylinositol to improve blood half-lives of liposomes. These findings were expounded upon by Gabizon et al. (Proc. Natl. Acad. Sci. U.S.A., (1988), 85,:6949). U.S. Pat. No. 4,837,028 and WO 88/04924, both to Allen et al., disclose liposomes comprising (1) sphingomyelin and (2) the ganglioside Gi or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver RNAi agents to macrophages.

Further advantages of liposomes include: liposomes obtained from natural phospholipids are biocompatible and biodegradable; liposomes can incorporate a wide range of water and lipid soluble drugs; liposomes can protect encapsulated RNAi agents in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of RNAi agent (see, e.g., Felgner, P. L. et al., (1987) Proc. Natl. Acad. Sci. USA 8:7413-7417, and U.S. Pat. No. 4,897,355 for a description of DOTMA and its use with DNA).

A DOTMA analogue. 1,2-bis(olcoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(olcoyloxy)-3.3-(trimethylammonia)propane (“DOTAP”) (Bochringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (sec, e.g., U.S. Pat. No. 5,171,678).

Another cationic lipid conjugate includes derivatization of the lipid with cholesterol (“DC-Chol”) which has been formulated into liposomes in combination with DOPE (Sec, Gao, X. and Huang. L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer RNAi agent into the skin. In some implementations, liposomes are used for delivering RNAi agent to epidermal cells and also to enhance the penetration of RNAi agent into dermal tissues, e.g., into skin. For example, the liposomes can be applied topically. Topical delivery of drugs formulated as liposomes to the skin has been documented (sec, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang. C. Y. and Huang. L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with RNAi agent are useful for treating a dermatological disorder.

Liposomes that include RNAi agents can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include RNAi agent can be delivered, for example, subcutaneously by infection in order to deliver RNAi agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

Other formulations amenable to the present disclosure are described in U.S. provisional application Ser. No. 61/018,616, filed Jan. 2, 2008; 61/018,611, filed Jan. 2, 2008; 61/039.748, filed Mar. 26, 2008; 61/047,087, filed Apr. 22, 2008 and 61/051,528, filed May 8, 2008. PCT application number PCT/US2007/080331, filed Oct. 3, 2007, also describes formulations that are amenable to the present disclosure.

Transfersomes, yet another type of liposomes, are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin. Surfactants find wide application in formulations such as those described herein, particularlay in emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in Pharmaceutical Dosage Forms, Marcel Dekker, Inc., New York, N.Y., 1988, p. 285).

The RNAi agent for use in the methods of the disclosure can also be provided as micellar formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C22 alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol or m-cresol may be added to the mixed micellar composition to stabilize the formulation and protect against bacterial growth. Alternatively, phenol or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### B. Lipid Particles

RNAi agents, e.g., dsRNAs of in the disclosure may be fully encapsulated in a lipid formulation, e.g., a LNP, or other nucleic acid-lipid particle.

As used herein, the term “LNP” refers to a stable nucleic acid-lipid particle. LNPs typically contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; United States Patent publication No. 2010/0324120 and WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the disclosure.

Certain specific LNP formulations for delivery of RNAi agents have been described in the art, including, e.g., “LNP01” formulations as described in, e.g., WO 2008/042973, which is hereby incorporated by reference.

Additional exemplary lipid-dsRNA formulations are identified in the below.

| | | cationic lipid/non-cationic |
|---|---|---|
| | | lipid/cholesterol/PEG-lipid conjugate |
| | Ionizable/Cationic Lipid | Lipid:siRNA ratio |
| SNALP-1 | 1,2-Dilinolenyloxy-N,N- | DLinDMA/DPPC/holesterol/PEG- |
| | dimethylaminopropane (DLinDMA) | cDMA |
| | | (57.1/7.1/34.4/1.4) |
| | | lipid:siRNA ~7:1 |
| 2-XTC | 2,2-Dilinoleyl-4-dimethylaminoethyl-[1,3]- | XTC/DPPC/Cholesterol/PEG-cDMA |
| | dioxolane (XTC) | 57.1/7.1/34.4/1.4 |
| | | lipid:siRNA ~7:1 |
| LNP05 | 2,2-Dilinoleyl-4-dimethylaminoethyl-[1,3]- | XTC/DSPC/Cholesterol/PEG-DMG |
| | dioxolane (XTC) | 57.5/7.5/31.5/3.5 |
| | | lipid:siRNA ~6:1 |
| LNP06 | 2,2-Dilinoleyl-4-dimethylaminoethyl-[1,3]- | XTC/DSPC/Cholesterol/PEG-DMG |
| | dioxolane (XTC) | 57.5/7.5/31.5/3.5 |
| | | lipid:siRNA ~11:1 |
| LNP07 | 2,2-Dilinoleyl-4-dimethylaminoethyl-[1,3]- | XTC/DSPC/Cholesterol/PEG-DMG |
| | dioxolane (XTC) | 60/7.5/31/1.5, |
| | | lipid:siRNA ~6:1 |
| LNP08 | 2,2-Dilinoleyl-4-dimethylaminoethyl-[1,3]- | XTC/DSPC/Cholesterol/PEG-DMG |
| | dioxolane (XTC) | 60/7.5/31/1.5, |
| | | lipid:siRNA ~11:1 |
| LNP09 | 2,2-Dilinoleyl-4-dimethylaminoethyl-[1,3]- | XTC/DSPC/Cholesterol/PEG-DMG |
| | dioxolane (XTC) | 50/10/38.5/1.5 |
| | | Lipid:siRNA 10:1 |
| LNP10 | (3aR,5s,6aS)-N,N-dimethyl-2,2- | ALN100/DSPC/Cholesterol/PEG- |
| | di((9Z,12Z)-octadeca-9,12- | DMG |
| | dienyl)tetrahydro-3aH- | 50/10/38.5/1.5 |
| | cyclopenta[d][1,3]dioxol-5-amine | Lipid:siRNA 10:1 |
| | (ALN100) | |
| LNP11 | (6Z,9Z,28Z,31Z)-heptatriaconta-6,9,28,31- | MC-3/DSPC/Cholesterol/PEG-DMG |
| | tetraen-19-yl 4-(dimethylamino)butanoate | 50/10/38.5/1.5 |
| | (MC3) | Lipid:siRNA 10:1 |
| LNP12 | 1,1′-(2-(4-(2-((2-(bis(2- | Tech G1/DSPC/Cholesterol/PEG- |
| | hydroxydodecyl)amino)ethyl)(2- | DMG |
| | hydroxydodecyl)amino)ethyl)piperazin-1- | 50/10/38.5/1.5 |
| | yl)ethylazanediyl)didodecan-2-ol (Tech | Lipid:siRNA 10:1 |
| | G1) | |
| LNP13 | XTC | XTC/DSPC/Chol/PEG-DMG |
| | | 50/10/38.5/1.5 |
| | | Lipid:siRNA: 33:1 |
| LNP14 | MC3 | MC3/DSPC/Chol/PEG-DMG |
| | | 40/15/40/5 |
| | | Lipid:siRNA: 11:1 |
| LNP15 | MC3 | MC3/DSPC/Chol/PEG-DSG/GalNAc- |
| | | PEG-DSG |
| | | 50/10/35/4.5/0.5 |
| | | Lipid:siRNA: 11:1 |
| LNP16 | MC3 | MC3/DSPC/Chol/PEG-DMG |
| | | 50/10/38.5/1.5 |
| | | Lipid:siRNA: 7:1 |
| LNP17 | MC3 | MC3/DSPC/Chol/PEG-DSG |
| | | 50/10/38.5/1.5 |
| | | Lipid:siRNA: 10:1 |
| LNP18 | MC3 | MC3/DSPC/Chol/PEG-DMG |
| | | 50/10/38.5/1.5 |
| | | Lipid:siRNA: 12:1 |
| LNP19 | MC3 | MC3/DSPC/Chol/PEG-DMG |
| | | 50/10/35/5 |
| | | Lipid:siRNA: 8:1 |
| LNP20 | MC3 | MC3/DSPC/Chol/PEG-DPG |
| | | 50/10/38.5/1.5 |
| | | Lipid:siRNA: 10:1 |
| LNP21 | C12-200 | C12-200/DSPC/Chol/PEG-DSG |
| | | 50/10/38.5/1.5 |
| | | Lipid:siRNA: 7:1 |
| LNP22 | XTC | XTC/DSPC/Chol/PEG-DSG |
| | | 50/10/38.5/1.5 |
| | | Lipid:siRNA: 10:1 |

DSPC: distearoylphosphatidylcholine
DPPC: dipalmitoylphosphatidylcholine
PEG-DMG: PEG-didimyristoyl glycerol (C14-PEG, or PEG-C14) (PEG with avg mol wt of 2000)
PEG-DSG: PEG-distyryl glycerol (C18-PEG, or PEG-C18) (PEG with avg mol wt of 2000)
PEG-cDMA: PEG-carbamoyl-1,2-dimyristyloxypropylamine (PEG with avg mol wt of 2000)
SNALP (1,2-Dilinolenyloxy-N,N-dimethylaminopropane (DLinDMA)) comprising formulations are described in WO 2009/127060, which is hereby incorporated by reference.
XTC comprising formulations are described in WO 2010/088537, the entire contents of which are hereby incorporated herein by reference.
MC3 comprising formulations are described, e.g., in United States Patent Publication No. 2010/0324120, the entire contents of which are hereby incorporated by reference.
ALNY-100 comprising formulations are described in WO 2010/054406, the entire contents of which are hereby incorporated herein by reference.
C12-200 comprising formulations are described in WO 2010/129709, the entire contents of which are hereby incorporated herein by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the disclosure are administered in conjunction with one or more penetration enhancer surfactants and chelators. Suitable surfactants include fatty acids or esters or salts thereof, bile acids or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the disclosure can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, U.S. 2003/0027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions can be generated from a variety of components that include, but are not limited to, preformed liquids, self-emulsifying solids and self-emulsifying semisolids. Particularly preferred are formulations that target the brain when treating APP-associated diseases or disorders.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

### C. Additional Formulations

i. Emulsions

The compositions of the present disclosure can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in cither aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N. Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N. Y., volume 1, p. 199). Emulsion formulations for oral delivery have been very widely used because of case of formulation, as well as efficacy from an absorption and bioavailability standpoint (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present disclosure, the compositions of RNAi agents and nucleic acids are formulated as microemulsions. A microemulsion can be defined as a system of water, oil and amphiphile which is a single optically isotropic and thermodynamically stable liquid solution (sec e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used, and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, case of preparation, case of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or RNAi agents. Microemulsions have also been effective in the transdermal delivery of active components in both cosmetic and pharmaceutical applications. It is expected that the microemulsion compositions and formulations of the present disclosure will facilitate the increased systemic absorption of RNAi agents and nucleic acids from the gastrointestinal tract, as well as improve the local cellular uptake of RNAi agents and nucleic acids.

Microemulsions of the present disclosure can also contain additional components and additives such as sorbitan monostearate (Grill 3), Labrasol, and penetration enhancers to improve the properties of the formulation and to enhance the absorption of the RNAi agents and nucleic acids of the present disclosure. Penetration enhancers used in the microemulsions of the present disclosure can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

An RNAi agent of the disclosure may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present disclosure employs various penetration enhancers to effect the efficient delivery of nucleic acids, particularly RNAi agents, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p.92). Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of RNAi agents through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, NY. 2002; Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C1 20 alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, M A, 2006; Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, NY, 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, NY, 2002; Lec et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present disclosure, can be defined as compounds that remove metallic ions from solution by forming complexes therewith, with the result that absorption of RNAi agents through the mucosa is enhanced. With regards to their use as penetration enhancers in the present disclosure, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, M A, 2006; Lec et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of RNAi agents through the alimentary mucosa (sce e.g., Muranishi, Critical Reviews in Therapeutic Drug Carrier Systems, 1990, 7, 1-33). This class of penetration enhancers includes, for example, unsaturated cyclic urcas, 1-alkyl- and 1-alkenylazacyclo-alkanone derivatives (Lec et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of RNAi agents at the cellular level can also be added to the pharmaceutical and other compositions of the present disclosure. For example, cationic lipids, such as lipofectin (Junichi et al, U.S. Pat. No. 5,705,188), cationic glycerol derivatives, and polycationic molecules, such as polylysine (WO 97/30731), are also known to enhance the cellular uptake of dsRNAs.

Other agents can be utilized to enhance the penetration of the administered nucleic acids, including glycols such as ethylene glycol and propylene glycol, pyrrols such as 2-pyrrol, azones, and terpenes such as limonene and menthone.

vi. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

vii. Other Components

The compositions of the present disclosure can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings or aromatic substances and the like which do not deleteriously interact with the nucleic acid(s) of the formulation.

Aqueous suspensions can contain substances which increase the viscosity of the suspension including, for example, sodium carboxymethylcellulose, sorbitol or dextran. The suspension can also contain stabilizers.

In some embodiments, pharmaceutical compositions featured in the disclosure include (a) one or more RNAi agents and (b) one or more agents which function by a non-RNAi mechanism and which are useful in treating a C9orf72-associated disorder. Examples of such agents include, but are not limited to, monoamine inhibitors, reserpine, anticonvulsants, antipsychotic agents, and antidepressants.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agents featured in the disclosure can be administered in combination with other known agents effective in treatment of pathological processes mediated by nucleotide repeat expression. In any event, the administering physician can adjust the amount and timing of RNAi agent administration on the basis of results observed using standard measures of efficacy known in the art or described herein.

### VII. Kits

In certain aspects, the instant disclosure provides kits that include a suitable container containing a pharmaceutical formulation of a siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof).

Such kits include one or more dsRNA agent(s) and instructions for use, e.g., instructions for administering a prophylactically or therapeutically effective amount of a dsRNA agent(s). The dsRNA agent may be in a vial or a pre-filled syringe. The kits may optionally further comprise means for administering the dsRNA agent (e.g., an injection device, such as a pre-filled syringe), or means for measuring the inhibition of C9orf72 (e.g., means for measuring the inhibition of C9orf72 mRNA, C9orf72 protein, and/or C9orf72 activity). Such means for measuring the inhibition of C9orf72 may comprise a means for obtaining a sample from a subject, such as, e.g., a CSF and/or plasma sample. The kits of the invention may optionally further comprise means for determining the therapeutically effective or prophylactically effective amount.

In certain embodiments the individual components of the pharmaceutical formulation may be provided in one container. Alternatively, it may be desirable to provide the components of the pharmaceutical formulation separately in two or more containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### VII. Methods for Inhibiting C9orf72 Expression

The present disclosure also provides methods of inhibiting the expression or reducing the level of a C9orf72 gene or a transcript associated with the C9orf72 locus in a cell. The methods include contacting a cell with an RNAi agent, e.g., double stranded RNAi agent, in an amount effective to inhibit the expression or reducing the level of C9orf72 or a transcript associated with the C9orf72 locus in the cell, thereby inhibiting the expression or reducing the level of C9orf72 or reducing the amount of the transcript associated with the C9orf72 locus in the cell. In certain embodiments of the disclosure, C9orf72 is inhibited preferentially in CNS (e.g., brain) cells.

In some embodiments, the methods include contacting a cell with two or more dsRNA agents targeting C9orf72. In certain embodiments of the methods including two or more dsRNA agents, the two or more dsRNA agents may be present in the same composition, in separate compositions, or any combination thereof.

In one embodiment of the methods which include contacting a cell with two or more dsRNA agents targeting C9orf72, at least one dsRNA agent targets an antisense strand of C9orf72 and at least one dsRNA agent targets a sense strand of C9orf72.

In some embodiments, suitable agents targeting a sense strand of C9orf72 for use in the methods of the invention comprising two or more dsRNA agents comprise a sense strand an antisense strand forming a double stranded region selected from the group consisting of
a) an antisense strand comprising at least 15 contiguous nucleotides differing by no more than 3 nucleotides from any one of the antisense nucleotide sequences in any one of Tables 5, 6, 10B, and 10D;
b) a sense strand comprising at least 15 contiguous nucleotides differing by no more than three nucleotides from any one of the nucleotide sequence of nucleotides 1-23; 15-37; 33-55; 37-59; or 62-84 of SEQ ID NO: 1, and an antisense strand comprising at least 15 contiguous nucleotides from the corresponding nucleotide sequence of SEQ ID NO:5;
c) a sense strand comprising at least 15 contiguous nucleotides differing by no more than three nucleotides from any one of the nucleotide sequence of nucleotides 5197-5219; 5226-5248; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and an antisense strand comprising at least 15 contiguous nucleotides from the corresponding nucleotide sequence of SEQ ID NO:16;
d) a sense strand comprising at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and an antisense strand comprising at least 15 contiguous nucleotides from the corresponding nucleotide sequence of SEQ ID NO:16;
e) a sense strand comprising at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and an antisense strand comprising at least 15 contiguous nucleotides from the corresponding nucleotide sequence of SEQ ID NO:5; and
f) an antisense strand comprising at least 15 contiguous nucleotides differing by no more than 3 nucleotides from any one of the antisense nucleotide sequences in any one of Tables 8, 9, 10B, and 10D,
wherein the sense strand, the antisense strand, or both the sense strand and the antisense strand is conjugated to one or more lipophilic moieties.

In certain embodiments, suitable agents targeting a sense strand of C9orf72, e.g., of a C9orf72 exon or intron sense sequence, for use in the methods of the invention comprising two or more dsRNA agents are those dsRNA agents disclosed in PCT Publication No. WO 2021/119226, the entire contents of which are incorporated herein by reference.

In certain embodiments, suitable agents targeting an antisense strand of C9orf72 for use in the methods of the invention comprising two or more dsRNA agents comprise a sense strand an an antisense strand forming a double stranded region selected from the group consisting of
a) a sense strand comprising at least 15 contiguous nucleotides differing by no more than 3 nucleotides from the nucleotide sequence of SEQ ID NO:13 and an antisense strand comprising a nucleotide sequence comprising at least 15 contiguous nucleotides differing by no more than 3 nucleotides from the corresponding portion of the nucleotide sequence of SEQ ID NO:14,
b) an antisense comprising a nucleotide sequence selected from the group consisting of any of the antisense strand nucleotide sequences in any one of Tables 2, 3, 10A, 10C. 11, and 12; and
c) an antisense strand comprising at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13;
d) a sense strand comprising at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and an antisense strand comprising at least 15 contiguous nucleotides from the corresponding nucleotide sequence of SEQ ID NO: 14,
wherein the sense strand, the antisense strand, or both the sense strand and the antisense strand is conjugated to one or more lipophilic moieties.

In some embodiments, the methods of the invention include contacting a cell with a composition comprising two or more, e.g., 2, 3, or 4, dsRNA agents of the invention, e.g., any two or more of the dsRNA agents selected from the group of dsRNA agents in Tables 2, 3, 5, 6, 8, 9, 10A, 10B, 10C, 10D, 11, and 12.

In some embodiments of the methods of the invention which include contacting a cell with two or more dsRNA agents, as described herein, e.g., any two or more, e.g., 2, 3, or 4, of the dsRNA agents selected from the group of dsRNA agents in Tables 2, 3, 5, 6, 8, 9, 10A, 10B, 10C, 10D, 11, and 12, the cell may be contacted with a first agent (or a composition comprising a first agent) at a first time, a second agent (or a composition comprising a second agent) at a second time, a third agent (or a composition comprising a third agent) at a third time, and a fourth agent (or a composition comprising a fourth agent) at a fourth time; or the cell may be contacted with all of the agents (or a composition comprising all of the agents) at the same time. Alternatively, the cell may be contacted with a first agent (or a composition comprising a first agent) at a first time and a second, third, and/or fourth agent (or a composition comprising a second, third, and/or fourth agent) at a second time. Other combinations of contacting the cell with two or more agents (or compositions comprising two or more dsRNA agents) of the invention are also contemplated.

Contacting of a cell with an RNAi agent, e.g., a double stranded RNAi agent, may be done in vitro or in vivo. Contacting a cell in vivo with the RNAi agent includes contacting a cell or group of cells within a subject, e.g., a human subject, with the RNAi agent. Combinations of in vitro and in vivo methods of contacting a cell are also possible.

Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc ligand, or any other ligand that directs the RNAi agent to a site of interest.

The term “inhibiting.” as used herein, is used interchangeably with “reducing.” “silencing.” “downregulating.” “suppressing” and other similar terms, and includes any level of inhibition. In certain embodiments, a level of inhibition, e.g., for an RNAi agent of the instant disclosure, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., about 50%, can thereby be identified as indicative of “inhibiting” or “reducing”, “downregulating” or “suppressing”, etc. having occurred. It is expressly contemplated that assessment of targeted mRNA or encoded protein levels (and therefore an extent of “inhibiting”, etc. caused by an RNAi agent of the disclosure) can also be assessed in in vivo systems for the RNAi agents of the instant disclosure, under properly controlled conditions as described in the art.

The phrase “inhibiting expression of a C9orf72 gene” or “inhibiting expression of C9orf72.” as used herein, includes inhibition of the expression, or reducing the level, of any C9orf72 gene (such as, e.g., a mouse C9orf72 gene, a rat C9orf72 gene, a monkey C9orf72 gene, or a human C9orf72 gene) as well as variants or mutants of a C9orf72 gene that encode a C9orf72 protein, e.g., a C9orf72 gene having an expanded hexanucleotide repeat in an intron of the gene. Thus, the C9orf72 gene may be a wild-type C9orf72 gene, a mutant C9orf72 gene, or a transgenic C9orf72 gene in the context of a genetically manipulated cell, group of cells, or organism.

The phrase “reducing the level or amount of the transcript associated with the C9orf72 locus” or “knocking down a transcript associated with the C9orf72 locus” includes inhibition of expression of or reducing the level or amount in a cell of an antisense strand of C9orf72 or a sense strand of C9orf72 (such as, e.g., a C9orf72 sense strand or antisense strand transcript containing a hexanucleotide repeat expansion).

“Inhibiting expression of a C9orf72 gene” includes any level of inhibition of a C9orf72 gene, e.g., at least partial suppression of the expression of a C9orf72 gene, such as an inhibition by at least 20%. In certain embodiments, inhibition is by at least 30%, at least 40%, or preferably, by at least 50%. In other embodiments, inhibition is no more than 50%, e.g., no more than 50%, 45%, 40%, 35%, 30%, 25%, 20%, 15%, 10%, or 5%.

The expression of a C9orf72 gene may be assessed based on the level of any variable associated with C9orf72 gene expression, e.g., C9orf72 mRNA level (e.g., sense mRNA, antisense mRNA, total C9orf72 mRNA, sense C9orf72 repeat-containing mRNA, and/or antisense C9orf72 repeat-containing mRNA) or C9orf72 protein level (e.g., total C9orf72 protein, wild-type C9orf72 protein, or expanded repeat-containing protein), or, for example, the level of sense- or antisense-containing foci and/or the level of aberrant dipeptide repeat protein.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

For example, in some embodiments of the methods of the disclosure, expression of a C9orf72 gene (e.g., as assessed by sense- or antisense-containing foci and/or aberrant dipeptide repeat protein level) is inhibited by at least 20%, 30%, 40%, 50%, 60%, 70%, 80%, 85%, 90%, or 95%, or to below the level of detection of the assay. In other embodiments of the methods of the disclosure, expression of a C9orf72 gene (e.g., as assessed by mRNA or protein expression level) is inhibited by no more than 50%, 45%, 40%, 35%, 30%, 25%, 20%, 15%, 10%, or 5%. In certain embodiments, the methods include a clinically relevant inhibition of expression of C9orf72, e.g. as demonstrated by a clinically relevant outcome after treatment of a subject with an agent to reduce the expression of C9orf72.

Inhibition of the expression of a C9orf72 gene may be manifested by a reduction of the amount of mRNA expressed by a first cell or group of cells (such cells may be present, for example, in a sample derived from a subject) in which a C9orf72 gene is transcribed and which has or have been treated (e.g., by contacting the cell or cells with an RNAi agent of the disclosure, or by administering an RNAi agent of the disclosure to a subject in which the cells are or were present) such that the expression of a C9orf72 gene is inhibited, as compared to a second cell or group of cells substantially identical to the first cell or group of cells but which has not or have not been so treated (control cell(s) not treated with an RNAi agent or not treated with an RNAi agent targeted to the gene of interest). The degree of inhibition may be expressed in terms of:

$$\frac{( RNA in control cells ) - ( RNA in treated cells )}{RNA in control cells} \times 100 \%$$

In other embodiments, inhibition of the expression of a C9orf72 gene may be assessed in terms of a reduction of a parameter that is functionally linked to a C9orf72 gene expression, e.g., C9orf72 protein expression, sense- or antisense-containing foci and/or the level of aberrant dipeptide repeat protein. C9orf72 gene silencing may be determined in any cell expressing C9orf72, either endogenous or heterologous from an expression construct, and by any assay known in the art.

Inhibition of the expression of a C9orf72 protein may be manifested by a reduction in the level of the C9orf72 protein (or functional parameter, e.g., as described herein) that is expressed by a cell or group of cells (e.g., the level of protein expressed in a sample derived from a subject). As explained above, for the assessment of mRNA suppression, the inhibiton of protein expression levels in a treated cell or group of cells may similarly be expressed as a percentage of the level of protein in a control cell or group of cells.

A control cell or group of cells that may be used to assess the inhibition of the expression of a C9orf72 gene includes a cell or group of cells that has not yet been contacted with an RNAi agent of the disclosure. For example, the control cell or group of cells may be derived from an individual subject (e.g., a human or animal subject) prior to treatment of the subject with an RNAi agent.

The level of C9orf72 mRNA that is expressed by a cell or group of cells may be determined using any method known in the art for assessing mRNA expression. In one embodiment, the level of expression of C9orf72 in a sample is determined by detecting a transcribed polynucleotide, or portion thereof, e.g., mRNA of the C9orf72 gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Strand specific C9orf72 mRNAs may be detected using the quantitative RT-PCR and, or droplet digital PCR methods described in, for example, Jiang, et al. supra, Lagier-Tourenne, et al., supra and Jiang, et al., supra. Circulating C9orf72 mRNA may be detected using methods the described in WO2012/177906, the entire contents of which are hereby incorporated herein by reference.

In some embodiments, the level of expression of C9orf72 is determined using a nucleic acid probe. The term “probe”, as used herein, refers to any molecule that is capable of selectively binding to a specific C9orf72 nucleic acid or protein, or fragment thereof. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to C9orf72 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of C9orf72 mRNA.

An alternative method for determining the level of expression of C9orf72 in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of C9orf72 is determined by quantitative fluorogenic RT-PCR (i.e., the TaqMan™ System), by a Dual-Glo® Luciferase assay, or by other art-recognized method for measurement of C9orf72 expression or mRNA level.

The expression level of C9orf72 mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of C9orf72 expression level may also comprise using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of C9orf72 nucleic acids.

The level of C9orf72 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of C9orf72 proteins.

The level of sense- or antisense-containing foci and the level of aberrant dipeptide repeat protein may be assessed using methods well-known to one of ordinary skill in the art, including, for example, fluorescent in situ hybridization (FISH), immunohistochemistry and immunoassay (see, e.g., Jiang, et al. supra). In some embodiments, the efficacy of the methods of the disclosure in the treatment of a C9orf72-associated disease is assessed by a decrease in C9orf72 mRNA level (e.g. by assessment of a CSF sample and/or plasma sample for C9orf72 level, by brain biopsy, or otherwise).

In some embodiments of the methods of the disclosure, the RNAi agent is administered to a subject such that the RNAi agent is delivered to a specific site within the subject. The inhibition of expression of C9orf72 may be assessed using measurements of the level or change in the level of C9orf72 mRNA (e.g., sense mRNA, antisense mRNA, total C9orf72 mRNA, sense C9orf72 repeat-containing mRNA, and/or antisense C9orf72 repeat-containing mRNA), C9orf72 protein(e.g., total C9orf72 protein, wild-type C9orf72 protein, or expanded repeat-containing protein), sense-containing foci, antisense-containing foci, aberrant dipeptide repeat protein in a sample derived from a specific site within the subject, e.g., CNS cells. In certain embodiments, the methods include a clinically relevant inhibition of expression of C9orf72, e.g. as demonstrated by a clinically relevant outcome after treatment of a subject with an agent to reduce the expression of C9orf72, suchas, for example, stabilization or inhibition of caudate atrophy (e.g., as assessed by volumetric MRI (vMRI)), a stabilization or reduction in neurofilament light chain (Nfl) levels in a CSF sample from a subject, a reduction in mutant C9orf72 mRNA or a cleaved mutant C9orf72 protein, e.g., one or both of full-length mutant C9orf72 mRNA or protein and a cleaved mutant C9orf72 mRNA or protein, and a stabilization or improvement in Unified C9orf72-associated disease Rating Scale (UHDRS) score.

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

### IX. Methods of Treating or Preventing C9orf72-Associated Diseases

The methods disclosed herein provide for the therapeutic reduction in the synthesis of dipeptide repeat proteins, a principle pathogenic component of C9orf72 repeat expansion discasc, while sparing the C9orf72 mRNA, thereby avoiding possible adverse effects of reduction of C9orf72 protein, as could occur with therapeutic strategies, such as the use of antisense oligonucleotides, that target the primary C9orf72 transcript in the nucleus.

Some of the methods disclosed herein are for inhibiting expression or reducing the level of a C9orf72 target RNA comprising a hexanucleotide repeat comprising multiple contiguous copies of the hexanucleotide repeat in a cell. The C9orf72 target RNA can be, for example, one with a pathogenic hexanucleotide repeat expansion (having, for example, at least about 30, at least about 35, at least about 40, at least about 50, at least about 60, at least about 70, at least about 80, at least about 100, at least about 200, at least about 300, at least about 400, or at least about 500 copies of the hexanucleotide repeat). Such methods can comprise introducing into the cell any of the dsRNA agents disclosed herein, thereby inhibiting expression of the C9orf72 target RNA in the cell.

Thus, the present disclosure also provides methods of using an RNAi agent of the disclosure or a composition (such as a pharmaceutical composition) containing an RNAi agent of the disclosure to reduce the level of one or more C9orf72 RNA transcripts in a cell. The methods include contacting the cell with a dsRNA, two or more dsRNA agents, e.g., 2, 3, or 4, of the disclosure, a composition (such as a pharmaceutical compostion) comprising two or more, e.g., 2, 3, or 4, dsRNA agent of the disclosure, or two or more, e.g., 2, 3, or 4, compositions (such as pharmaceutical compositions), each independently comprising a dsRNA agent of the invention, and maintaining the cell for a time sufficient to obtain degradation of the mRNA transcript of a C9orf72 gene, thereby reducing the level of one or more the C9orf72 RNA transcripts in the cell.

In addition, the present disclosure also provides methods of using an RNAi agent of the disclosure or a composition (such as a pharmaceutical compostion) containing an RNAi agent of the disclosure to reduce the level and/or inhibit formation of sense- and antisense-containing foci in a cell. The methods include contacting the cell with a dsRNA of the disclosure, two or more dsRNAs, e.g., 2, 3, or 4, of the disclosure, a composition (such as a pharmaceutical compostion) comprising two or more, e.g., 2, 3, or 4, dsRNAs of the disclosure, or two or more, e.g., 2, 3, or 4, compositions (such as pharmaceutical compositions), each independently comprising a dsRNA agent of the invention, thereby reducing the level of the C9orf72 sense- and antisense-containing foci in the cell.

The present disclosure also provides methods of using an RNAi agent of the disclosure or a composition (such as a pharmaceutical compostion) containing an RNAi agent of the disclosure to reduce the level and/or inhibit formation of aberrant dipeptide repeat protein in a cell. The methods include contacting the cell with a dsRNA of the disclosure, two or more dsRNA, e.g., 2, 3, or 4, of the disclosure, a composition (such as a pharmaceutical compostion) comprising two or more, e.g., 2, 3, or 4, dsRNA of the disclosure, or two or more, e.g., 2, 3, or 4, compositions (such as pharmaceutical compositions), each independently comprising a dsRNA agent of the invention, thereby reducing the level of the aberrant dipeptide repeat protein in the cell.

Such methods can further comprise assessing expression of the C9orf72 target RNA in the cell and/or assessing expression of a mature C9orf72 mRNA in the cell. The assessing can be done, for example, by reverse-transcription quantitative polymerase chain reactions to detect the C9orf72 target RNA. However, any other suitable method may be used.

In the methods of the disclosure the cell may be contacted in vitro or in vivo, i.e., the cell may be within a subject.

In some embodiments, the methods include contacting a cell with two or more dsRNA agents targeting C9orf72. In certain embodiments of the methods including two or more dsRNA agents, the two or more dsRNA agents may be present in the same composition, in separate compositions, or any combination thereof.

In one embodiment of the methods which include contacting a cell with two or more dsRNA agents targeting C9orf72, at least one dsRNA agent which targets an antisense strand of C9orf72 and at least one dsRNA agent which targets a sense strand of C9orf72.

In some embodiments, suitable agents targeting a sense strand of C9orf72 for use in the methods of the invention comprising two or more dsRNA agents comprise a sense strand and an antisense strand forming a double stranded region selected from the group consisting of
a) a sense strand comprising at least 15 contiguous nucleotides differing by no more than 3 nucleotides from the nucleotide sequence of SEQ ID NO:1 and an antisense strand comprising a nucleotide sequence comprising at least 15 contiguous nucleotides differing by no more than 3 nucleotides from the corresponding portion of the nucleotide sequence of SEQ ID NO:5,
b) a sense strand comprising at least 15 contiguous nucleotides differing by no more than 3 nucleotides from the nucleotide sequence of SEQ ID NO:15 and an antisense strand comprising a nucleotide sequence comprising at least 15 contiguous nucleotides differing by no more than 3 nucleotides from the corresponding portion of the nucleotide sequence of SEQ ID NO:16,
c) an antisense strand comprising at least 15 contiguous nucleotides differing by no more than 3 nucleotides from any one of the antisense nucleotide sequences in any one of Tables 5, 6, 10B, and 10D;
d) a sense strand comprising at least 15 contiguous nucleotides differing by no more than three nucleotides from any one of the nucleotide sequence of nucleotides 1-23; 15-37; 33-55; 37-59; 62-84, or 69-91 of SEQ ID NO: 1, and an antisense strand comprising at least 15 contiguous nucleotides from the corresponding nucleotide sequence of SEQ ID NO:5;
c) a sense strand comprising at least 15 contiguous nucleotides differing by no more than three nucleotides from any one of the nucleotide sequence of nucleotides 5197-5219; 5223-5245; 5226-5248; 5227-5249; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and an antisense strand comprising at least 15 contiguous nucleotides from the corresponding nucleotide sequence of SEQ ID NO:16;
f) a sense strand comprising at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and an antisense strand comprising at least 15 contiguous nucleotides from the corresponding nucleotide sequence of SEQ ID NO:16;
g) a sense strand comprising at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and an antisense strand comprising at least 15 contiguous nucleotides from the corresponding nucleotide sequence of SEQ ID NO:5; and
h) an antisense strand comprising at least 15 contiguous nucleotides differing by no more than 3 nucleotides from any one of the antisense nucleotide sequences in any one of Tables 8 and 9,
wherein the sense strand, the antisense strand, or both the sense strand and the antisense strand comprises at least one modified nucleotide.

In some embodiments, the sense strand, the antisense strand, or both the sense and the antisense strand is conjugated to one or more lipophilic moieties.

In certain embodiments, suitable agents targeting a sense strand of C9orf72, e.g, of a C9orf72 exon or intron sense sequence, for use in the methods of the invention comprising two or more dsRNA agents are those dsRNA agents disclosed in PCT Publication No. WO 2021/119226, the entire contents of which are incorporated herein by reference.

In certain embodiments, suitable agents targeting an antisense strand of C9orf72 for use in the methods of the invention comprising two or more dsRNA agents comprise a sense strand an an antisense strand forming a double stranded region selected from the group consisting of
a) a sense strand comprising at least 15 contiguous nucleotides differing by no more than 3 nucleotides from the nucleotide sequence of SEQ ID NO:13 and an antisense strand comprising a nucleotide sequence comprising at least 15 contiguous nucleotides differing by no more than 3 nucleotides from the corresponding portion of the nucleotide sequence of SEQ ID NO: 14,
b) a sense strand comprising at least 15 contiguous nucleotides differing by no more than 3 nucleotides from the nucleotide sequence of SEQ ID NO: 17 and an antisense strand comprising a nucleotide sequence comprising at least 15 contiguous nucleotides differing by no more than 3 nucleotides from the corresponding portion of the nucleotide sequence of SEQ ID NO:18,
c) a sense strand comprising at least 15 contiguous nucleotides differing by no more than 3 nucleotides from the nucleotide sequence of SEQ ID NO:19 and an antisense strand comprising a nucleotide sequence comprising at least 15 contiguous nucleotides differing by no more than 3 nucleotides from the corresponding portion of the nucleotide sequence of SEQ ID NO:20,
d) an antisense comprising a nucleotide sequence selected from the group consisting of any of the antisense strand nucleotide sequences in any one of Tables 2, 3, 10A, 10C, 11, and 12; and
e) a sense strand comprising at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13;
f) a sense strand comprising at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and an antisense strand comprising at least 15 contiguous nucleotides from the corresponding nucleotide sequence of SEQ ID NO:14,
wherein the sense strand, the antisense strand, or both the sense strand and the antisense strand comprises at least one modified nucleotide.

In some embodiments, the methods of the invention include contacting a cell with a two or more, e.g., 2, 3, or 4, dsRNA agents of the invention, e.g., any two or more of the dsRNA agents selected from the group of dsRNA agents in Tables 2, 3, 5, 6, 8, 9, 10A, 10B, 10C, 10D, 11, and 12.

In some embodiments, the sense strand, the antisense strand, or both the sense and the antisense strand is conjugated to one or more lipophilic moieties.

A cell suitable for treatment using the methods of the disclosure may be any cell that expresses a C9orf72 gene or a cell that expresses a C9orf72 gene having an expanded hexanucleotides (e.g., GGGGCC) repeat (SEQ ID NO: 100). A cell suitable for use in the methods of the disclosure may be a mammalian cell, e.g., a primate cell (such as a human cell or a non-human primate cell, e.g., a monkey cell or a chimpanzee cell), a non-primate cell (such as a a rat cell, or a mouse cell). In one embodiment, the cell is a human cell, e.g., a human CNS cell. In some embodiments, the cell is a non-human animal one-cell stage embryos, non-human animal embryonic stem cells, embryonic stem-cell derived motor neurons, brain cells, cortical cells, neuronal cells, muscle cells, heart cells, or germ cells.

In some embodiments, the cell can comprise a C9orf72 locus comprising a pathogenic hexanucleotide repeat expansion. A pathogenic hexanucleotide repeat expansion is an expansion consisting of a number of repeats of GGGGCC (SEQ ID NO: 100) in an intervening sequence separating two putative first, non-coding exons (exons 1A and 1B) in the gene C9orf72 that is associated with one or both of the following pathological readouts: (1) sense and antisense repeat-containing RNA can be visualized as distinct foci in neurons and other cells; and (2) dipeptide repeat proteins-poly(glycine-alanine), poly(glycine-proline), poly(glycine-arginine), poly(alanine-proline), and poly(proline-arginine)-synthesized by repeat-associated non-AUG-dependent translation from the sense and antisense repeat-containing RNA can be detected in cells. The number of repeats can be a higher number of repeats than is normally seen in a locus from someone that does not have C9orf72 ALS or C9orf72 FTD. Alternatively, a pathogenic hexanucleotide repeat expansion can be an expansion (i.e., number of repeats) in a C9orf72 locus from a subject having C9orf72 ALS or C9orf72 FTD. A pathogenic hexanucleotide repeat expansion has a plurality of repeats of GGGGCC (SEQ ID NO: 100). For example, a pathogenic hexanucleotide repeat expansion can have, for example, at least about 30, at least about 35, at least about 40, at least about 50, at least about 60, at least about 70, at least about 80, at least about 100, at least about 200, at least about 300, at least about 400, or at least about 500 copies of the hexanucleotide repeat.

The cell can be a cell (e.g. a neuron or a motor neuron) from a subject having, or at risk for developing, a C9orf72-hexanucleotide-repeat-expansion associated disease including, for example, C9orf72 ALS or C9orf72 FTD.

The cells in the methods disclosed herein can be any type of cell comprising a C9orf72 locus. The C9orf72 locus can comprise a hexanucleotide repeat expansion sequence or a pathogenic hexanucleotide repeat expansion sequence as described elsewhere herein. The hexanucleotide repeat expansion sequence may comprise more than 100 repeats of the hexanucleotide sequence set forth as SEQ ID NO: 100.

A C9orf72 hexanucleotide repeat expansion sequence is generally a nucleotide sequence comprising at least two instances (i.e., two repeats) of the hexanucleotide sequence GGGGCC set forth as SEQ ID NO: 100. In some hexanucleotide repeat expansion sequences, the repeats are contiguous (adjacent to each other without intervening sequence). The repeat expansion sequence can be located, for example, between the first non-coding endogenous exon and exon 2 of the endogenous C9orf72 locus.

The hexanucleotide repeat expansion sequence can have any number of repeats. For example, the repeat expansion sequence can comprise more than about 95 repeats, more than about 96 repeats, more than about 97 repeats, more than about 98 repeats, more than about 99 repeats, more than about 100 repeats, more than about 101 repeats, more than about 102 repeats, more than about 103 repeats, more than about 104 repeats, more than about 105 repeats, more than about 150 repeats, more than about 200 repeats, more than about 250 repeats, more than about 295 repeats, more than about 296 repeats, more than about 297 repeats, more than about 298 repeats, more than about 299 repeats, more than about 300 repeats, more than about 301 repeats, more than about 302 repeats, more than about 303 repeats, more than about 304 repeats, more than about 305 repeats, more than about 350 repeats, more than about 400 repeats, more than about 450 repeats, more than about 500 repeats, more than about 550 repeats, more than about 595 repeats, more than about 596 repeats, more than about 597 repeats, more than about 598 repeats, more than about 599 repeats, more than about 600 repeats, more than about 601 repeats, more than about 602 repeats, more than about 603 repeats, more than about 604 repeats, or more than about 605 repeats. Alternatively, the repeat expansion sequence can comprise at least about 95 repeats, at least about 96 repeats, at least about 97 repeats, at least about 98 repeats, at least about 99 repeats, at least about 100 repeats, at least about 101 repeats, at least about 102 repeats, at least about 103 repeats, at least about 104 repeats, at least about 105 repeats, at least about 150 repeats, at least about 200 repeats, at least about 250 repeats, at least about 295 repeats, at least about 296 repeats, at least about 297 repeats, at least about 298 repeats, at least about 299 repeats, at least about 300 repeats, at least about 301 repeats, at least about 302 repeats, at least about 303 repeats, at least about 304 repeats, at least about 305 repeats, at least about 350 repeats, at least about 400 repeats, at least about 450 repeats, at least about 500 repeats, at least about 550 repeats, at least about 595 repeats, at least about 596 repeats, at least about 597 repeats, at least about 598 repeats, at least about 599 repeats, at least about 600 repeats, at least about 601 repeats, at least about 602 repeats, at least about 603 repeats, at least about 604 repeats, or at least about 605 repeats. In a specific example, the hexanucleotide repeat expansion sequence comprises more than about 100 repeats, more than about 300 repeats, more than about 600 repeats, at least about 100 repeats, at least about 300 repeats, or at least about 600 repeats.

The cells can be in vitro, ex vivo, or in vivo. For example, the cells can be in vivo within an animal. The cells or animals can be male or female. The cells or animals can be heterozygous or homozygous for the hexanucleotide repeat expansion sequence inserted at the endogenous C9orf72 locus. A diploid organism has two alleles at each genetic locus. Each pair of alleles represents the genotype of a specific genetic locus. Genotypes are described as homozygous if there are two identical alleles at a particular locus and as heterozygous if the two alleles differ. The non-human animals can comprise the heterologous hexanucleotide repeat expansion sequence inserted at the endogenous C9orf72 locus in their germline genome.

C9orf72 expression (e.g., as assessed by sense mRNA, antisense mRNA, total C9orf72 mRNA, sense C9orf72 repeat-containing mRNA, antisense C9orf72 repeat-containing mRNA level, total C9orf72 protein, and/or C9orf72 repeat-containing protein) is inhibited in the cell by about 20, 25, 30, 35, 40, 45, or 50%. In preferred embodiments, C9orf72 expression is inhibited by no more than 50%, e.g., no more than 50%, 45%, 40%, 35%, 30%, 25%, 20%, 15%, 10%, or 5%.

The decrease in expression in the C9orf72 target RNA can be by any amount. Inhibition, as assessed by sense- or antisense-containing foci and/or aberrant dipeptide repeat protein level) is inhibited in the cell by at least 20%, 30%, 40%, preferably at least 50%, 60%, 70%, 80%, 85%, 90%, or 95%, or to below the level of detection of the assay.

In some embodiments, the dsRNA agent may inhibit expression of the C9orf72 target RNA, such as a C9orf72 target RNA comprising a hexanucleotide repeat, by at least about 10%, at least about 20%, at least about 30%, at least about 40%, at least about 50%, at least about 60%, at least about 70%, at least about 80%, or at least about 90% (or to a point where the C9orf72 target RNA is undetectable). For example, these levels of inhibition can be within about 1 day, within about 2 days, within about 3 days, within about 4 days, within about 5 days, within about 6 days, within about a week, or within about 24 to about 48 hours after administration to a cell expressing the C9orf72 target RNA comprising the hexanucleotide repeat. The decrease can be, for example, relative to the cell before treatment with dsRNA agent or relative to a control cell that was not treated with the dsRNA agent.

In some of the methods, the dsRNA agents of the invention selectively inhibit expression of the C9orf72 target RNA, such as a C9orf72 target RNA comprising a hexanucleotide repeat, relative to expression of a mature C9orf72 messenger RNA. A mature C9orf72 messenger RNA in this context is a C9orf72 RNA transcript that has been spliced and processed. A mature C9orf72 messenger RNA consists exclusively of exons and has all introns removed. A dsRNA agent selectively inhibits expression of the C9orf72 target RNA comprising the intronic hexanucleotide repeat relative to expression of a mature C9orf72 messenger RNA if the relative decrease in expression of the C9orf72 target RNA is greater than the relative decrease in expression of a mature C9orf72 messenger RNA after administration of the dsRNA agent to a cell expressing the C9orf72 target RNA. For example, in certain embodiments, dsRNA agents of the invention inhibit expression of the mature C9orf72 messenger RNA by less than about 50%, less than about 40%, less than about 30%, less than about 20%, less than about 10%, or less than about 5% (or, for example, does not have any statistically significant or functionally significant effect on expression). For example, these levels of inhibition can be within about 1 day, within about 2 days, within about 3 days, within about 4 days, within about 5 days, within about 6 days, within about a week, or within about 24 to about 48 hours after administration to a cell expressing the C9orf72 target RNA comprising the hexanucleotide repeat. The decrease can be, for example, relative to the cell before treatment with the dsRNA agent or relative to a control cell that was not treated with the dsRNA agent.

Some of the methods disclosed herein are for reducing dipeptide repeat protein synthesis or dipeptide repeat protein aggregates in a cell. Such methods can comprise introducing into the cell any of the dsRNA agents disclosed herein, two or more dsRNA, e.g., 2, 3, or 4, of the disclosure, a composition (such as a pharmaceutical compostion) comprising two or more, e.g., 2, 3, or 4, dsRNA agents of the disclosure, or two or more, e.g., 2, 3, or 4, compositions (such as pharmaceutical compositions), each independently comprising a dsRNA agent of the invention, thereby reducing dipeptide repeat protein synthesis or dipeptide repeat protein aggregates in the cell.

Such methods can further comprise assessing the presence of dipeptide repeat protein aggregates (e.g., poly(glycine-alanine), poly(glycine-proline), poly(glycine-arginine), poly(alanine-proline), and poly(proline-arginine)) in the cell. In a specific example, the dipeptide repeat protein can be poly(glycine-alanine) and/or poly(glycine-proline). The assessing can be done, for example, by immunohistochemistry or western blot analysis to detect the dipeptide repeat protein aggregates. However, any other suitable method may be used.

The decrease in dipeptide repeat protein synthesis or dipeptide repeat protein aggregates can be by any amount. For example, the dsRNA agent can reduce dipeptide repeat protein synthesis or dipeptide repeat protein aggregates by at least about 10%, at least about 20%, at least about 30%, at least about 40%, at least about 50%, at least about 60%, at least about 70%, at least about 80%, or at least about 90% (or to a point where the dipeptide repeat protein aggregates are undetectable). For example, these levels of inhibition can be within about 1 day, within about 2 days, within about 3 days, within about 4 days, within about 5 days, within about 6 days, within about a week, or within about 24 to about 48 hours after administration to a cell expressing the C9orf72 target RNA comprising the hexanucleotide repeat. The decrease can be, for example, relative to the cell before treatment with dsRNA agent or relative to a control cell that was not treated with the dsRNA agent.

Such methods can further comprise assessing the presence of nuclear and/or cytoplasmic sense and/or antisense C9orf72 RNA foci in the cell.

The decrease in the presence of nuclear and/or cytoplasmic sense and/or antisense C9orf72 RNA foci can be by any amount. For example, the dsRNA agent can reduce the presence of nuclear and/or cytoplasmic sense and/or antisense C9orf72 RNA foci by at least about 10%, at least about 20%, at least about 30%, at least about 40%, at least about 50%, at least about 60%, at least about 70%, at least about 80%, or at least about 90% (or to a point where the nuclear and/or cytoplasmic sense and/or antisense C9orf72 RNA foci are undetectable). For example, these levels of inhibition can be within about 1 day, within about 2 days, within about 3 days, within about 4 days, within about 5 days, within about 6 days, within about a week, or within about 24 to about 48 hours after administration to a cell expressing the C9orf72 target RNA comprising the hexanucleotide repeat. The decrease can be, for example, relative to the cell before treatment with dsRNA agent or relative to a control cell that was not treated with the dsRNA agent.

The in vivo methods of the disclosure may include administering to a subject a composition containing an RNAi agent, where the RNAi agent includes a nucleotide sequence that is complementary to at least a part of an RNA transcript of the C9orf72 gene of the mammal to be treated. In some embodiments, the subject is administered two or more, e.g., 2, 3, or 4, compositions, each independently comprising an RNAi agent of the invention. The compositions may be the same or different. In other embodiments, the subject is administered a composition comprising two or more, e.g., 2, 3, or 4, dsRNA agents, each independently targeting a portion of a C9orf72 gene.

When the organism to be treated is a mammal such as a human, the composition can be administered by any means known in the art including, but not limited to oral, intraperitoneal, or parenteral routes, including intracranial (e.g., intraventricular, intraparenchymal, and intrathecal), intravenous, intramuscular, intravitreal, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intrathecal injection.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of C9orf72, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intracranial, intravenous, subcutaneous, arterial, or epidural infusions. In preferred embodiments, the infusion pump is a subcutaneous infusion pump. In other embodiments, the pump is a surgically implanted pump that delivers the RNAi agent to the CNS.

The mode of administration may be chosen based upon whether local or systemic treatment is desired and based upon the area to be treated. The route and site of administration may be chosen to enhance targeting.

In one aspect, the present disclosure also provides methods for inhibiting the expression of a C9orf72 gene in a mammal. The methods include administering to the mammal a composition comprising a dsRNA that targets a C9orf72 gene in a cell of the mammal, thereby inhibiting expression of the C9orf72 gene in the cell. In some embodiments, the dsRNA is present in a composition, such as a pharmaceutical composition. In some embodiments, the mammal is administered two or more, e.g., 2, 3, or 4, dsRNA agents of the invention. In some embodiments, each dsRNA agent administered to the subject is independently present in a composition. In other embodiments, the mammal is administered a composition comprising two or more, e.g., 2, 3, or 4, dsRNAs of the invention.

Reduction in gene expression can be assessed by any methods known it the art and by methods, e.g. qRT-PCR, described herein.

Reduction in protein production can be assessed by any methods known it the art and by methods, e.g. ELISA, described herein. In one embodiment, a CNS biopsy sample or a cerebrospinal fluid (CSF) sample serves as the tissue material for monitoring the reduction in C9orf72 gene or protein expression (or of a proxy therefore).

The present disclosure further provides methods of treatment of a subject in need thereof. The treatment methods of the disclosure include administering an RNAi agent of the disclosure to a subject, e.g., a subject that would benefit from inhibition of C9orf72 expression, such as a subject having a GGGGCC expanded nucleotide repeat (SEQ ID NO: 100) in an intron of the C9orf72 gene, in a therapeutically effective amount of an RNAi agent targeting a C9orf72 gene or a pharmaceutical composition comprising an RNAi agent targeting a C9orf72 gene. In some embodiments, the subject is administered a therapeutically effective amount of two or more, e.g., 2, 3, or 4, dsRNA agents of the invention. In some embodiments, each dsRNA agent administered to the subject is independently present in a composition. In other embodiments, the subject is administered a composition comprising two or more, e.g., 2, 3, or 4, dsRNAs of the invention.

In addition, the present disclosure provides methods of preventing, treating or inhibiting the progression of a C9orf72-associated disease or disorder (e.g., a C9orf72-associated disorder), in a subject. The methods include administering to the subject a therapeutically effective amount of any of the RNAi agent, e.g., dsRNA agents, or the pharmaceutical composition provided herein, thereby preventing, treating or inhibiting the progression of a C9orf72-associated disease or disorder in the subject. In some embodiments, the subject is administered a therapeutically effective amount of two or more, e.g., 2, 3, or 4, dsRNA agents of the invention. In some embodiments, each dsRNA agent administered to the subject is independently present in a composition. In other embodiments, the subject is administered a composition comprising two or more, e.g., 2, 3, or 4, dsRNAs of the invention.

In some embodiments, the methods are for treating a subject suffering from a C9orf72-hexanucleotide-repeat-expansion-associated disease, condition, or disorder. Such methods can also be for preventing or ameliorating at least one symptom in a subject having a disease, disorder, or condition that would benefit from reduction in expression of a C9orf72 target RNA comprising a hexanucleotide repeat comprising multiple contiguous copies of SEQ ID NO: 100 (e.g., a subject having or at risk of developing a C9orf72-hexanucleotide-repeat-expansion-associated disease, condition, or disorder). The C9orf72 target RNA can be, for example, one with a pathogenic hexanucleotide repeat expansion (having, for example, at least about 30, at least about 35, at least about 40, at least about 50, at least about 60, at least about 70, at least about 80, at least about 100, at least about 200, at least about 300, at least about 400, or at least about 500 copies of the hexanucleotide repeat). A C9orf72-hexanucleotide-repeat-expansion-associated disease, condition, or disorder is one in which caused by or associated with an expansion of a hexanucleotide repeat (GGGGCC; SEQ ID NO: 100) in the 5′ non-coding part of the C9orf72 gene. Examples include amyotrophic lateral sclerosis (ALS) and frontotemporal dementia (FTD). Signs or symptoms associated with FTD and/or ALS, include, but are not limited to, repeat-length-dependent formation of RNA foci, sequestration of specific RNA-binding proteins, and accumulation and aggregation of dipeptide repeat proteins (e.g., poly(glycine-alanine), poly(glycine-proline), poly(glycine-arginine), poly(alanine-proline), and poly(proline-arginine)) resulting from repeat-associated non-AUG (AUG) translation in neurons. The dsRNA agents of the invention may be used in methods for therapeutic treatment and/or prevention of signs or symptoms associated with FTD and/or ALS, including, but not limited to, signs and symptoms of motor neuron disease and signs and symptoms of dementia. Signs and symptoms of motor neuron disease can include, for example, tripping, dropping things, abnormal fatigue of the arms and/or legs, slurred speech, muscle cramps and twitches, uncontrollable periods of laughing or crying, and trouble breathing. Signs and symptoms of dementia can include, for example, behavioral changes, personality changes, speech and language problems, and movement-related problems.

In some embodiments of the methods of the invention which include administering two or more dsRNA agents, as described herein, e.g., any two or more, e.g., 2, 3, or 4, of the dsRNA agents selected from the group of dsRNA agents in Tables 2, 3, 5, 6, 8, 9, 10A, 10B, 10C, 10D, 11, and 12 the subject may be administered a first agent (or a composition comprising a first agent) at a first time, a second agent (or a composition comprising a second agent) at a second time, a third agent (or a compositions comprising a third agent) at a third time, and a fourth agent (or a composition comprising a fourth agent) at a fourth time; or the subject may be administered all of the agents (or a composition comprising all of the agents at the same time. Alternatively, the subject may be administered a first agent (or a composition comprising a first agent) at a first time and a second, third, and/or fourth agent (or a compostion comprising a second, third and.or fourth agent) at a second time. Other combinations of contacting the cell with two or more agents of the invention are also contemplated.

An RNAi agent of the disclosure may be administered as a “free RNAi agent.” A free RNAi agent is administered in the absence of a pharmaceutical composition. The naked RNAi agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject.

Alternatively, an RNAi agent of the disclosure may be administered as a pharmaceutical composition, such as a dsRNA liposomal formulation.

Subjects that would benefit from a reduction or inhibition of C9orf72 gene expression are those having a C9orf72-associated disease, e.g., C9orf72-associated disease. Exemplary C9orf72-associated diseases include, but are not limited to, ALS, FTD, C9ALS/FTD and Huntington-Like Syndrome Due To C9orf72 Expansions, parkinsonism, olivopontocerebellar degeneration, corticobasal syndrome, or Alzheimer's disease, e.g., subjects having an expanded GGGGCC hexanucleotide repeat (SEQ ID NO: 100) in an intron of the C9orf72 gene.

The disclosure further provides methods for the use of an RNAi agent or a pharmaceutical composition thereof, e.g., for treating a subject that would benefit from reduction or inhibition of C9orf72 expression, e.g., a subject having a C9orf72-associated disorder, in combination with other pharmaceuticals or other therapeutic methods, e.g., with known pharmaceuticals or known therapeutic methods, such as, for example, those which are currently employed for treating these disorders. For example, in certain embodiments, an RNAi agent targeting C9orf72 is administered in combination with, e.g., an agent useful in treating a C9orf72-associated disorder as described elsewhere herein or as otherwise known in the art. For example, additional agents suitable for treating a subject that would benefit from reduction in C9orf72 expression, e.g., a subject having a C9orf72-associated disorder, may include agents currently used to treat symptoms of C9orf72-associated diseases. The RNAi agent and additional therapeutic agents may be administered at the same time or in the same combination, e.g., intrathecally, or the additional therapeutic agent can be administered as part of a separate composition or at separate times or by another method known in the art or described herein.

Exemplary additional therapeutics include, for example, a monoamine inhibitor, e.g., tetrabenazine (Xenazine), deutetrabenazine (Austedo), and reserpine, an anticonvulsant, e.g., valproic acid (Depakote, Depakene, Depacon), and clonazepam (Klonopin), an antipsychotic agent, e.g., risperidone (Risperdal), and haloperidol (Haldol), and an antidepressant, e.g., paroxetine (Paxil).

In one embodiment, the method includes administering a composition featured herein such that expression of the target C9orf72 gene is decreased, for at least one month. In preferred embodiments, expression is decreased for at least 2 months, 3 months, or 6 months.

Preferably, the RNAi agents useful for the methods and compositions featured herein specifically target RNAs (primary or processed) of the target C9orf72 gene. Compositions and methods for inhibiting the expression of these genes using RNAi agents can be prepared and performed as described herein.

Administration of the dsRNA according to the methods of the disclosure may result in a reduction of the severity, signs, symptoms, or markers of such diseases or disorders in a patient with a C9orf72-associated disorder. By “reduction” in this context is meant a statistically significant or clinically significant decrease in such level. The reduction can be, for example, at least 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of a C9orf72-associated disorder may be assessed, for example, by periodic monitoring of a subject's. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi agent targeting C9orf72 or pharmaceutical composition thereof, “effective against” a C9orf72-associated disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating C9orf72-associated disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an RNAi agent or RNAi agent formulation as described herein.

Subjects can be administered a therapeutic amount of dsRNA, such as about 0.01 mg/kg to about 200 mg/kg.

The RNAi agent can be administered intrathecally, via intravitreal injection, or by intravenous infusion over a period of time, on a regular basis. In certain embodiments, after an initial treatment regimen, the treatments can be administered on a less frequent basis. Administration of the RNAi agent can reduce C9orf72 levels, e.g., in a cell, tissue, blood, CSF sample or other compartment of the patient. In one embodiment, administration of the RNAi agent can reduce C9orf72 levels, e.g., in a cell, tissue, blood, CSF sample or other compartment of the patient by no more than 50%.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the RNAi agent can be administered subcutaneously, i.e., by subcutaneous injection. One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimine may include administration of a therapeutic amount of RNAi agent on a regular basis, such as monthly or extending to once a quarter, twice per year, once per year. In certain embodiments, the RNAi agent is administered about once per month to about once per quarter (i.e., about once every three months).

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. Hexanucleotide Repeat Expansion at the C9orf72 Gene Locus

Amyotrophic lateral sclerosis (ALS) and frontotemporal dementia (FTD) are devastating neurodegenerative diseases that cause motor neuron disease in the case of ALS and dementia in the case of FTD. Both are invariably fatal. ALS and FTD can present as either a spontaneous or familial (i.e., genetic) disease. The most common genetic cause of ALS and FTD is an expansion of a hexanucleotide repeat (GGGGCC; SEQ ID NO: 100) in the 5′ non-coding part of the C9orf72 gene, which encodes a protein whose function is not fully understood. Unaffected people usually have between a few and a few dozen hexanucleotide repeats in their C9orf72 genes, while those that develop ALS and FTD inherit a repeat expansion of hundreds to thousands of copies of the hexanucleotide repeat from only one of their parents. Genetic observations suggest that C9orf72 ALS and FTD are dominant genetic diseases and result from a gain of pathological function.

It is not known how the C9orf72 hexanucleotide repeat expansion causes motor neuron disease and dementia, but two universal postmortem pathological findings in C9orf72 ALS and FTLD patients are associated with the repeat expansion: (1) sense and antisense repeat-containing RNA can be visualized as distinct foci in neurons and other cells by fluorescent in situ hybridization; and (2) dipeptide repeat proteins-poly(glycine-alanine), poly(glycine-proline), poly(glycine-arginine), poly(alanine-proline), and poly(proline-arginine)-synthesized by repeat-associated non-AUG-dependent translation from the sense and antisense repeat-containing RNAs-can be detected in cells by immunohistochemistry. One disease hypothesis proposes that the repeat-containing RNAs, visualized as foci, disrupt cellular RNA metabolism by sequestering RNA binding proteins. A second disease hypothesis posits that the dipeptide repeat proteins exert wide-spread toxic effects on RNA metabolism, proteostasis, and nucleocytoplasmic transport. Both pathogenic mechanisms could contribute to disease. If C9orf72 repeat-containing RNA transcripts, either on their own or as templates for translation of dipeptide repeat proteins, promote pathogenesis in ALS and FTLD, then a general therapeutic strategy would be to destroy GGGGCC repeat-containing RNA (SEQ ID NO: 100) (sense repeat-containing RNA) and/or GGCCCC repeat-containing RNA (antisense repeat-containing RNA) or abolish its ability to be translated into sense and/or antisense dipeptide repeat protein.

The C9orf72 gene produces transcripts from two transcription initiation sites. The upstream site initiates transcription with alternative non-coding exon 1A, while the downstream site initiates transcription with alternative exon 1B. Both exons 1A and 1B can be spliced to exon 2, which contains the start of the protein-coding sequence. The pathogenic hexanucleotide repeat expansion is located between exons 1A and 1B. Therefore, transcription initiated from exon 1A can produce repeat-containing RNAs, while initiation from exon 1B cannot, unless going in the antisense direction.

As described in PCT Application No.: PCT/US2020/064159, filed on Dec. 10, 2020, in order to model C9orf72 repeat expansion disease in mice, an allelic series was constructed in mouse embryonic stem (ES) cells in which a fragment from the human C9orf72 gene, including part of exon 1A, the intron sequence between 1A and 1B, all of exon 1B and part of the downstream intron, was placed precisely at its homologous position in one allele of the mouse C9orf72 gene. Sec, e.g., US 2018/0094267 and WO 2018/064600, each of which is herein incorporated by reference in its entirety for all purposes. A series of hexanucleotide repeat expansions were placed at the position found in the human gene that ranged from the normal three repeats up to the pathological 600 repeats.

Mouse ES cell clones carrying the different repeat expansions were differentiated into motor neurons in culture to study the effects of the expansions on a cell type relevant to ALS. In examining the transcripts produced from the genetically modified humanized C9orf72 alleles it was found that there was a switch from exon 1B spliced transcripts, which predominate in the three repeat normal control, to increased appearance of exon 1A spliced transcripts in the alleles with longer repeat expansions. It was also observed the accumulation of unspliced intron-containing transcripts whose abundance was directly correlated with the length of the hexanucleotide repeat expansion, suggesting a selfish feed-forward loop in which the longer the repeat expansion, the more repeat-containing transcripts are produced from the C9orf72 gene. Targeting the repeat-containing intronic transcripts for destruction or inactivation as templates for dipeptide repeat protein synthesis while sparing synthesis of the normal C9orf72 mRNA and protein would be expected to be a safe and effective therapeutic strategy for C9orf72 repeat expansion disease.

One possible approach to reducing C9orf72 repeat-containing RNAs is through the natural process of RNA interference, in which siRNAs direct cleavage of the target RNAs by the RNA-induced silencing complex followed by degradation of the RNA cleavage fragments by cellular nucleases. RNA interference is, however, a predominantly cytoplasmic process that would not be expected to act on RNAs retained in the nucleus. Intron-containing RNAs are usually short-lived, either as mRNA precursors, which are rapidly spliced into mature mRNAs, or as spliced-out introns, which are rapidly degraded in the nucleus. It is reasonable, therefore, to expect that intron-containing RNAs would not be available for targeting by RNA interference.

However, it has been demonstrated that siRNAs that targeted intron sequences adjacent to the GGGGCC repeat expansion (SEQ ID NO: 100) promoted reduced accumulation of intron-containing C9orf72 RNAs while having little to no effect on the C9orf72 mature mRNA. The intron-targeting siRNAs also reduced production of dipeptide repeat proteins. These unexpected experimental results indicate that the intron-containing RNAs that accumulate in cells with a C9orf72 hexanucleotide repeat expansion are susceptible to RNA interference. The results show that a significant fraction of the intron-containing C9orf72 RNAs responsible for dipeptide repeat protein synthesis resides in the cytoplasm. In contrast, siRNAs that targeted the C9orf72 mRNA protein coding sequence produced a strong knock down of the mRNA but had no effect on the intron-containing transcripts and did not appreciably reduce dipeptide repeat protein synthesis. The divergenee in results between the intron-targeting and mRNA-targeting siRNAs suggests that the two classes of targeted sequences are present on separate RNAs that are not covalently linked.

The methods and compositions disclosed herein provide for the therapeutic reduction in the synthesis of dipeptide repeat proteins, a principle pathogenic component of C9orf72 repeat expansion disease, while sparing the C9orf72 mRNA, thereby avoiding possible adverse effects of reduction of C9orf72 protein, as could occur with therapeutic strategies, such as the use of antisense oligonucleotides, that target the primary C9orf72 transcript in the nucleus.

### Example 2. RNAi Agent Design, Synthesis, Selection, and In Vitro Evaluation

This Example describes methods for the design, synthesis, selection, and in vitro evaluation of C9orf72 RNAi agents.

### Source of Reagents

Where the source of a reagent is not specifically given herein, such reagent can be obtained from any supplier of reagents for molecular biology at a quality/purity standard for application in molecular biology.

### Bioinformatics

siRNAs targeting the antisense strand of the intron between Exons 1A and 1B in the human C9orf72 gene (GenBank Accession Number NC_000009.12) were designed using custom R and Python scripts. Detailed lists of the unmodified C9orf72 sense and antisense strand nucleotide sequences are shown in Table 2. Detailed lists of the modified C9orf72 sense and antisense strand nucleotide sequences are shown in Table 3.

siRNAs targeting the sense strand of Exon 1A of the human C9orf72 gene (GenBank Accession Number NM_001256054.2) were designed using custom R and Python scripts, siRNAs targeting the 3′-end of the intronic repeat in the sense strand of the intron between Exons 1A and 1B in the human C9orf72 gene (GenBank Accession Number NG_031977.2) were also designed using custom R and Python scripts. Detailed lists of the unmodified C9orf72 sense and antisense strand nucleotide sequences are shown in Table 5. Detailed lists of the modified C9orf72 sense and antisense strand nucleotide sequences are shown in Table 6.

It is to be understood that, throughout the application, a duplex name without a decimal is equivalent to a duplex name with a decimal which merely references the batch number of the duplex. For example, AD-347430 is equivalent to AD-347430.1.

In vitro Cos-7 (Dual-Luciferase psiCHECK2 vector), BE(2)-C, and Neuro-2a screening

### Cell Culture and Transfections:

Cos-7 (ATCC) were transfected by adding 5 μl of 2 ng/μl, diluted in Opti-MEM, C9orf72 intron 1 psiCHECK2 vector (Blue Heron Biotechnology), 4.9 μl of Opti-MEM plus 0.1 μl of Lipofectamine 2000 per well (Invitrogen, Carlsbad CA, cat #11668-019) to 5 μl of siRNA duplexes per well, with 4 replicates of each siRNA duplex, into a 384-well plate, and incubated at room temperature for 15 minutes. Thirty-five μl of Dulbecco's Modified Eagle Medium (ThermoFisher) containing ˜1×10³ cells were then added to the siRNA mixture. Cells were incubated for 48 hours followed by Firefly (transfection control) and Renilla (fused to target sequence) luciferase measurements. Three dose experiments were performed at 10 nM, InM, and 0.1 nM.

Total RNA isolation using DYNABEADS mRNA Isolation Kit:

RNA was isolated using an automated protocol on a BioTek-EL406 platform using DYNABEADs (Invitrogen, cat #61012). Briefly, 70 μl of Lysis/Binding Buffer and 10 μl of lysis buffer containing 3 μl of magnetic beads were added to the plate with cells. Plates were incubated on an electromagnetic shaker for 10 minutes at room temperature and then magnetic beads were captured and the supernatant was removed. Bead-bound RNA was then washed 2 times with 150 μl Wash Buffer A and once with Wash Buffer B. Beads were then washed with 150 μl Elution Buffer, re-captured and supernatant removed.

cDNA synthesis using ABI High capacity cDNA reverse transcription kit (Applied Biosystems, Foster City, CA, Cat #4368813):

Ten μl of a master mix containing 1 μl 10X Buffer, 0.4 μl 25X dNTPs, 1 μl 10x Random primers, 0.5 μl Reverse Transcriptase, 0.5 μl RNase inhibitor and 6.6 μl of H₂O per reaction was added to RNA isolated above. Plates were sealed, mixed, and incubated on an electromagnetic shaker for 10 minutes at room temperature, followed by 2 h 37° C.

Real time PCR:

Two μl of cDNA and 5 μl Lightcycler 480 probe master mix (Roche Cat #04887301001) were added to either 0.5 μl of Human GAPDH TaqMan Probe (4326317E) and 0.5 μl C9orf72 Human probe (Hs00376619_m1, Thermo) or 0.5 μl Mouse GAPDH TaqMan Probe (4352339E) and 0.5 μl C9orf72 Mouse probe (Mm01216837_m1. Thermo) per well in a 384 well plates (Roche cat #04887301001). Real time PCR was done in a LightCycler480 Real Time PCR system (Roche). Each duplex was tested at least two times and data were normalized to cells transfected with a non-targeting control siRNA. To calculate relative fold change, real time data were analyzed using the AACt method and normalized to assays performed with cells transfected with a non-targeting control siRNA.

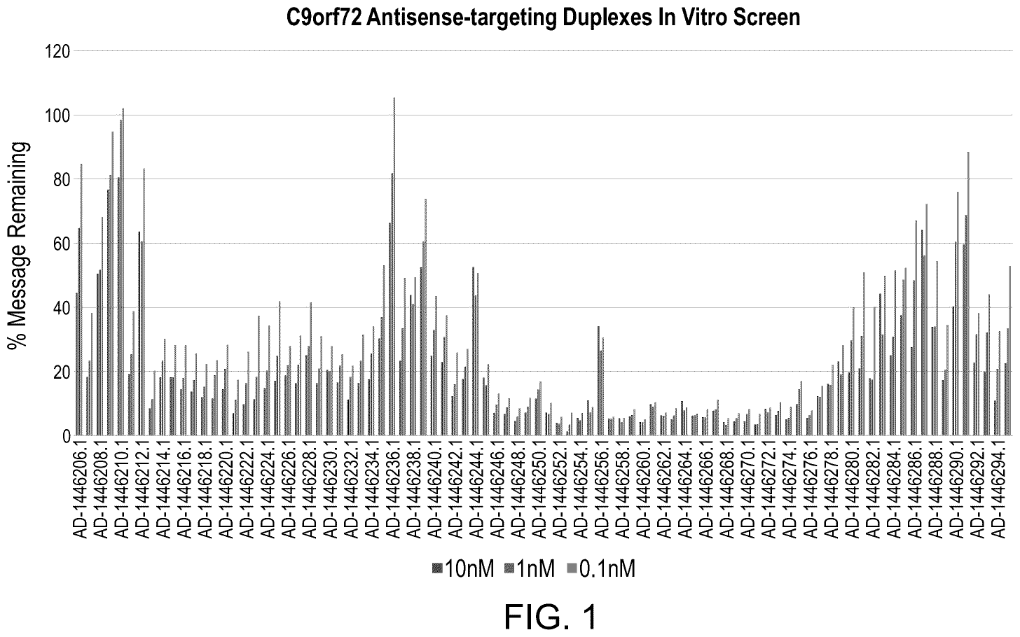

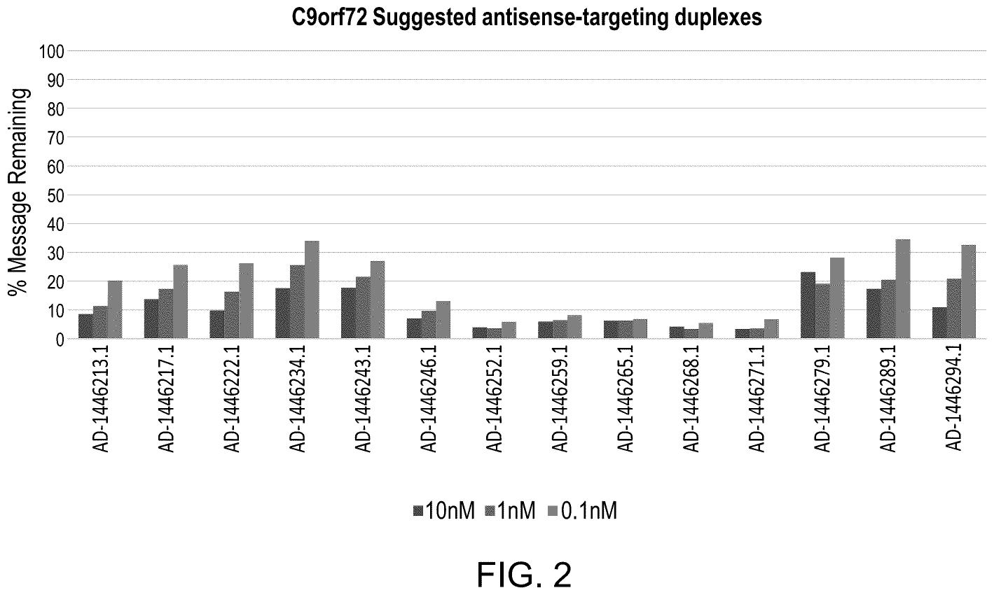

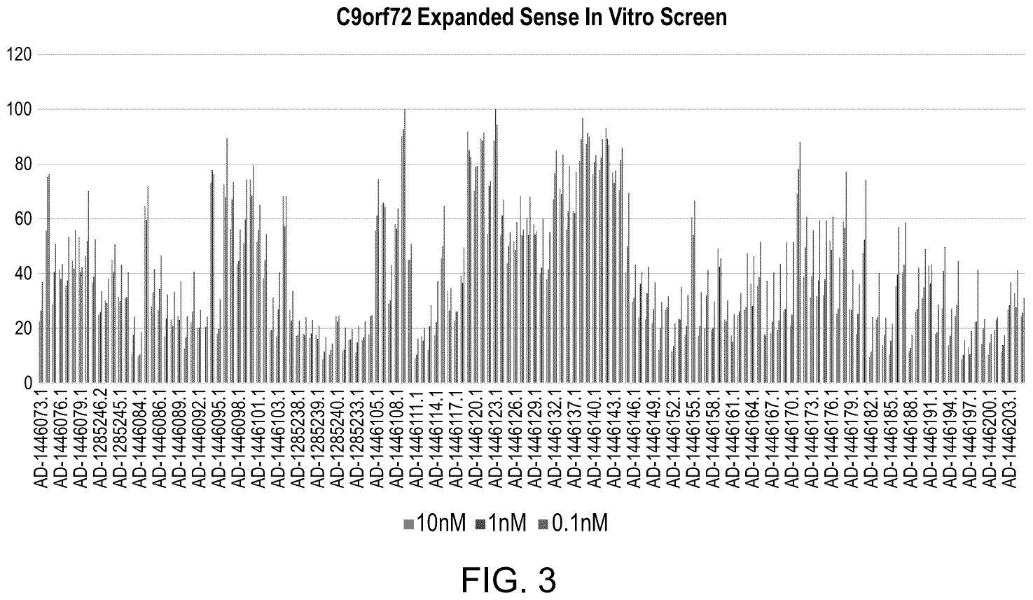

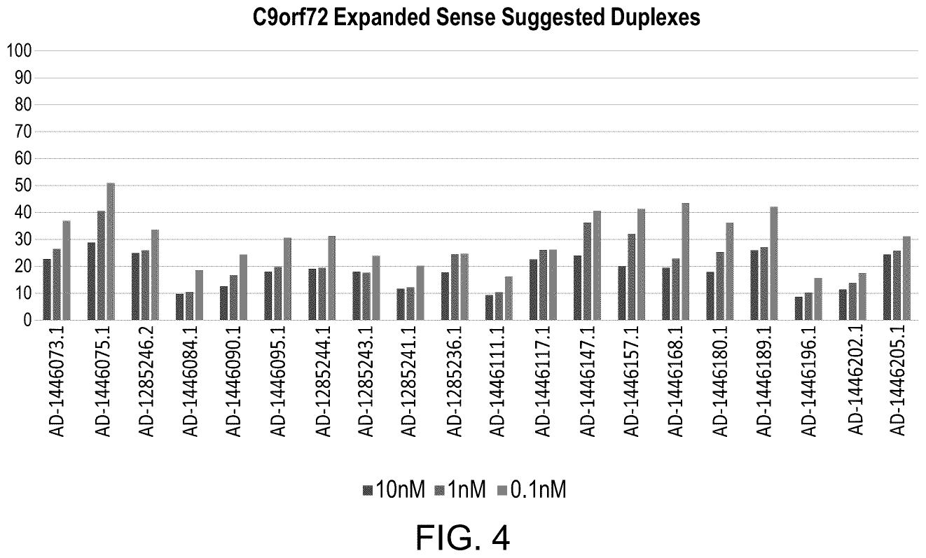

The results of the screening of the dsRNA agents listed in Tables 2 and 3 in Cos-7 cells are shown in Table 4 and FIGS. 1 and 2. The results of the screening of the dsRNA agents listed in Tables 5 and 6 in Cos-7 cells are shown in Table 7 and FIGS. 3 and 4.

TABLE 1
Abbreviations of nucleotide monomers used in nucleic acid sequence representation. It will
be understood that these monomers, when present in an oligonucleotide, are mutually
linked by 5′-3′-phosphodiester bonds.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine -3′-phosphorothioate |
| Us | uridine -3′-phosphorothioate |
| N | any nucleotide, modified or unmodified |
| a | 2′-O-methyladenosine-3′-phosphate |
| as | 2′-O-methyladenosine-3′- phosphorothioate |
| c | 2′-O-methylcytidine-3′-phosphate |
| cs | 2′-O-methylcytidine-3′- phosphorothioate |
| g | 2′-O-methylguanosine-3′-phosphate |
| gs | 2′-O-methylguanosine-3′- phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol Hyp-(GalNAc-alkyl)3 |
| | 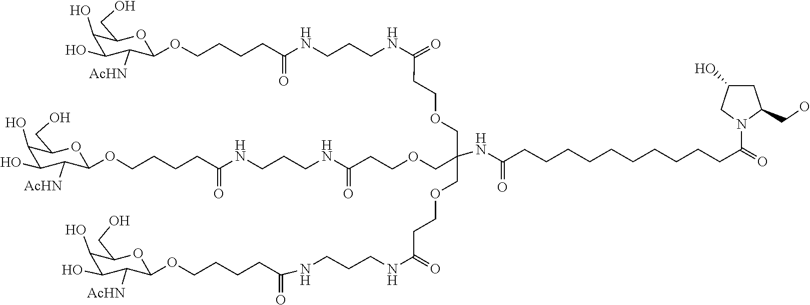 |
| Y34 | 2-hydroxymethyl-tetrahydrofuran-4-methoxy-3-phosphate (abasic 2′-OMe furanose) |
| | 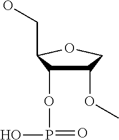 |
| Y44 | inverted abasic DNA (2-hydroxymethyl-tetrahydrofuran-5-phosphate) |
| | 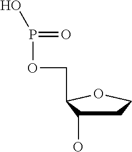 |
| L10 | N-(cholesterylcarboxamidocaproyl)-4-hydroxyprolinol (Hyp-C6-Chol) |
| | 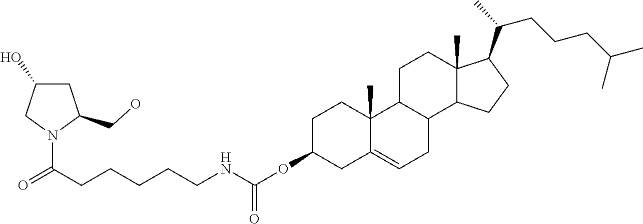 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |

TABLE 2
Unmodified Sense and Antisense Strand Sequences of dsRNA Agents Targeting the Antisense Strand of Intron 1a of Human C9orf72

| | | SEQ | | Range in | | SEQ | | |
|---|---|---|---|---|---|---|---|---|
| Duplex | Sense Sequence | ID | Range in | NC_0000 | Antisense Sequence | ID | Range in | Range in |
| Name | 5′ to 3′ | NO: | NG_31977.2 | 09.12 | 5′ to 3′ | NO: | NG_31977.2 | NC_000009.12 |
| AD- | CCGAGGCUCCCUUUUCUCG | 109 | 5761-5781 | 27573086- | UUCGAGAAAAGGGAGCCUCG | 199 | 5761-5783 | 27573084- |
| 1446206.1 | AA | | | 27573106 | GGU | | | 27573106 |
| AD- | AGGCAAUUCCACCAGUCGC | 110 | 5591-5611 | 27573256- | UAGCGACUGGUGGAAUUGCC | 200 | 5591-5613 | 27573254- |
| 1446207.1 | UA | | | 27573276 | UGC | | | 27573276 |
| AD- | CACCAGUCGCUAGAGGCG | 111 | 5582-5602 | 27573265- | UUUCGCCUCUAGCGACUGGU | 1408 | 5582-5604 | 27573263- |
| 1446208.1 | AAA | | | 27573285 | GGA | | | 27573285 |
| AD- | ACCAGUCGCUAGAGGCGA | 112 | 5581-5601 | 27573266- | UUUUCGCCUCUAGCGACUGG | 202 | 5581-5603 | 27573264- |
| 1446209.1 | AAA | | | 27573286 | UGG | | | 27573286 |
| AD- | CACCCAGCUUCGGUCAGAG | 113 | 5555-5575 | 27573292- | UUCUCUGACCGAAGCUGGGU | 1409 | 5555-5577 | 27573290- |
| 1446210.1 | AA | | | 27573312 | GUC | | | 27573312 |
| AD- | CCCAGCUUCGGUCAGAGA | 114 | 5553-5573 | 27573294- | UUUUCUCUGACCGAAGCUGG | 204 | 5553-5575 | 27573292- |
| 1446211.1 | AAA | | | 27573314 | GUG | | | 27573314 |
| AD- | CAGCUUCGGUCAGAGAAA | 115 | 5551-5571 | 27573296- | UCAUUUCUCUGACCGAAGCU | 205 | 5551-5573 | 27573294- |
| 1446212.1 | UGA | | | 27573316 | GGG | | | 27573316 |
| AD- | GCUUCGGUCAGAGAAAUG | 116 | 5549-5569 | 27573298- | UCUCAUUUCUCUGACCGAAG | 1410 | 5549-5571 | 27573296- |
| 1446213.1 | AGA | | | 27573318 | CUG | | | 27573318 |
| AD- | UCGGUCAGAGAAAUGAGA | 117 | 5546-5566 | 27573301- | UCCUCUCAUUUCUCUGACCG | 207 | 5546-5568 | 27573299- |
| 1446214.1 | GGA | | | 27573321 | AAG | | | 27573321 |
| AD- | GGUCAGAGAAAUGAGAGG | 118 | 5544-5564 | 27573303- | UUCCCUCUCAUUUCUCUGACC | 1411 | 5544-5566 | 27573301- |
| 1446215.1 | GAA | | | 27573323 | GA | | | 27573323 |
| AD- | UCAGAGAAAUGAGAGGGA | 119 | 5542-5562 | 27573305- | UUUUCCCUCUCAUUUCUCUG | 209 | 5542-5564 | 27573303- |
| 1446216.1 | AAA | | | 27573325 | ACC | | | 27573325 |
| AD- | AGAGGGAAAGUAAAAAUG | 120 | 5531-5551 | 27573316- | UCGCAUUUUUACUUUCCCUC | 210 | 5531-5553 | 27573314- |
| 1446217.1 | CGA | | | 27573336 | UCA | | | 27573336 |
| AD- | GAGGGAAAGUAAAAAUGC | 121 | 5530-5550 | 27573317- | UACGCAUUUUUACUUUCCCU | 211 | 5530-5552 | 27573315- |
| 1446218.1 | GUA | | | 27573337 | CUC | | | 27573337 |
| AD- | AGGGAAAGUAAAAAUGCG | 122 | 5529-5549 | 27573318- | UGACGCAUUUUUACUUUCCC | 212 | 5529-5551 | 27573316- |
| 1446219.1 | UCA | | | 27573338 | UCU | | | 27573338 |
| AD- | GGGAAAGUAAAAAUGCGU | 123 | 5528-5548 | 27573319- | UCGACGCAUUUUUACUUUCC | 213 | 5528-5550 | 27573317- |
| 1446220.1 | CGA | | | 27573339 | CUC | | | 27573339 |
| AD- | GGAAAGUAAAAAUGCGUC | 124 | 5527-5547 | 27573320- | UUCGACGCAUUUUUACUUUC | 214 | 5527-5549 | 27573318- |
| 1446221.1 | GAA | | | 27573340 | CCU | | | 27573340 |
| AD- | GAAAGUAAAAAUGCGUCG | 125 | 5526-5546 | 27573321- | UCUCGACGCAUUUUUACUUU | 215 | 5526-5548 | 27573319- |
| 1446222.1 | AGA | | | 27573341 | CCC | | | 27573341 |
| AD- | AAAGUAAAAAUGCGUCGA | 126 | 5525-5545 | 27573322- | UGCUCGACGCAUUUUUACUU | 216 | 5525-5547 | 27573320- |
| 1446223.1 | GCA | | | 27573342 | UCC | | | 27573342 |
| AD- | AAGUAAAAAUGCGUCGAG | 127 | 5524-5544 | 27573323- | UAGCUCGACGCAUUUUUACU | 217 | 5524-5546 | 27573321- |
| 1446224.1 | CUA | | | 27573343 | UUC | | | 27573343 |
| AD- | AGUAAAAAUGCGUCGAGC | 128 | 5523-5543 | 27573324- | UGAGCUCGACGCAUUUUUAC | 218 | 5523-5545 | 27573322- |
| 1446225.1 | UCA | | | 27573344 | UUU | | | 27573344 |
| AD- | CGACUCCUGAGUUCCAGA | 129 | 5293-5313 | 27573554- | UGCUCUGGAACUCAGGAGUC | 219 | 5293-5315 | 27573552- |
| 1446226.1 | GCA | | | 27573574 | GCG | | | 27573574 |
| AD- | GACUCCUGAGUUCCAGAG | 130 | 5292-5312 | 27573555- | UAGCUCTGGAACUCAGGAGU | 220 | 5292-5314 | 27573553- |
| 1446227.1 | CUA | | | 27573575 | CGC | | | 27573575 |
| AD- | ACUCCUGAGUUCCAGAGC | 131 | 5291-5311 | 27573556- | UAAGCUCUGGAACUCAGGAG | 221 | 5291-5313 | 27573554- |
| 1446228.1 | UUA | | | 27573576 | UCG | | | 27573576 |
| AD- | CUCCUGAGUUCCAGAGCU | 132 | 5290-5310 | 27573557- | UCAAGCTCUGGAACUCAGGA | 222 | 5290-5312 | 27573555- |
| 1446229.1 | UGA | | | 27573577 | GUC | | | 27573577 |
| AD- | UCCUGAGUUCCAGAGCUU | 133 | 5289-5309 | 27573558- | UGCAAGCUCUGGAACUCAGG | 223 | 5289-5311 | 27573556- |
| 1446230.1 | GCA | | | 27573578 | AGU | | | 27573578 |
| AD- | CCUGAGUUCCAGAGCUUG | 134 | 5288-5308 | 27573559- | UAGCAAGCUCUGGAACUCAG | 224 | 5288-5310 | 27573557- |
| 1446231.1 | CUA | | | 27573579 | GAG | | | 27573579 |
| AD- | GAGUUCCAGAGCUUGCUA | 135 | 5285-5305 | 27573562- | UUGUAGCAAGCUCUGGAACU | 225 | 5285-5307 | 27573560- |
| 1446232.1 | CAA | | | 27573582 | CAG | | | 27573582 |
| AD- | AGUUCCAGAGCUUGCUAC | 136 | 5284-5304 | 27573563- | UCUGUAGCAAGCUCUGGAAC | 226 | 5284-5306 | 27573561- |
| 1446233.1 | AGA | | | 27573583 | UCA | | | 27573583 |
| AD- | GUUCCAGAGCUUGCUACA | 137 | 5283-5303 | 27573564- | UCCUGUAGCAAGCUCUGGAA | 227 | 5283-5305 | 27573562- |
| 1446234.1 | GGA | | | 27573584 | CUC | | | 27573584 |
| AD- | UUCCAGAGCUUGCUACAG | 138 | 5282-5302 | 27573565- | UGCCUGTAGCAAGCUCUGGA | 228 | 5282-5304 | 27573563- |
| 1446235.1 | GCA | | | 27573585 | ACU | | | 27573585 |
| AD- | UCCAGAGCUUGCUACAGG | 139 | 5281-5301 | 27573566- | UAGCCUGUAGCAAGCUCUGG | 229 | 5281-5303 | 27573564- |
| 1446236.1 | CUA | | | 27573586 | AAC | | | 27573586 |
| AD- | CCAGAGCUUGCUACAGGC | 140 | 5280-5300 | 27573567- | UCAGCCTGUAGCAAGCUCUG | 230 | 5280-5302 | 27573565- |
| 1446237.1 | UGA | | | 27573587 | GAA | | | 27573587 |
| AD- | CAGAGCUUGCUACAGGCU | 141 | 5279-5299 | 27573568- | UGCAGCCUGUAGCAAGCUCU | 231 | 5279-5301 | 27573566- |
| 1446238.1 | GCA | | | 27573588 | GGA | | | 27573588 |
| AD- | UACAGGCUGCGGUUGUUU | 142 | 5269-5289 | 27573578- | UGGAAACAACCGCAGCCUGU | 232 | 5269-5291 | 27573576- |
| 1446239.1 | CCA | | | 27573598 | AGC | | | 27573598 |
| AD- | GCUGCGGUUGUUUCCCUCC | 143 | 5264-5284 | 27573583- | UAGGAGGGAAACAACCGCAG | 233 | 5264-5286 | 27573581- |
| 1446240.1 | UA | | | 27573603 | CCU | | | 27573603 |
| AD- | CUGCGGUUGUUUCCCUCCU | 144 | 5263-5283 | 27573584- | UAAGGAGGGAAACAACCGCA | 234 | 5263-5285 | 27573582- |
| 1446241.1 | UA | | | 27573604 | GCC | | | 27573604 |
| AD- | GCGGUUGUUUCCCUCCUU | 145 | 5261-5281 | 27573586- | UACAAGGAGGGAAACAACCG | 235 | 5261-5283 | 27573584- |
| 1446242.1 | GUA | | | 27573606 | CAG | | | 27573606 |
| AD- | CGGUUGUUUCCCUCCUUG | 146 | 5260-5280 | 27573587- | UAACAAGGAGGGAAACAACC | 236 | 5260-5282 | 27573585- |
| 1446243.1 | UUA | | | 27573607 | GCA | | | 27573607 |
| AD- | GUUGUUUCCCUCCUUGUU | 147 | 5258-5278 | 27573589- | UAAAACAAGGAGGGAAACAA | 237 | 5258-5280 | 27573587- |
| 1446244.1 | UUA | | | 27573609 | CCG | | | 27573609 |
| AD- | UUGUUUCCCUCCUUGUUU | 148 | 5257-5277 | 27573590- | UGAAAACAAGGAGGGAAACA | 238 | 5257-5279 | 27573588- |
| 1446245.1 | UCA | | | 27573610 | ACC | | | 27573610 |
| AD- | UUCCCUCCUUGUUUUCUUC | 149 | 5253-5273 | 27573594- | UAGAAGAAAACAAGGAGGGA | 239 | 5253-5275 | 27573592- |
| 1446246.1 | UA | | | 27573614 | AAC | | | 27573614 |
| AD- | UCCCUCCUUGUUUUCUUCU | 150 | 5252-5272 | 27573595- | UCAGAAGAAAACAAGGAGGG | 240 | 5252-5274 | 27573593- |
| 1446247.1 | GA | | | 27573615 | AAA | | | 27573615 |
| AD- | CCCUCCUUGUUUUCUUCUG | 151 | 5251-5271 | 27573596- | UCCAGAAGAAAACAAGGAGG | 241 | 5251-5273 | 27573594- |
| 1446248.1 | GA | | | 27573616 | GAA | | | 27573616 |
| AD- | CCUCCUUGUUUUCUUCUG | 152 | 5250-5270 | 27573597- | UACCAGAAGAAAACAAGGAG | 242 | 5250-5272 | 27573595- |
| 1446249.1 | GUA | | | 27573617 | GGA | | | 27573617 |
| AD- | CUCCUUGUUUUCUUCUGG | 153 | 5249-5269 | 27573598- | UAACCAGAAGAAAACAAGGA | 243 | 5249-5271 | 27573596- |
| 1446250.1 | UUA | | | 27573618 | GGG | | | 27573618 |
| AD- | CCUUGUUUUCUUCUGGUU | 154 | 5247-5267 | 27573600- | UUUAACCAGAAGAAAACAAG | 244 | 5247-5269 | 27573598- |
| 1446251.1 | AAA | | | 27573620 | GAG | | | 27573620 |
| AD- | CUUGUUUUCUUCUGGUUA | 155 | 5246-5266 | 27573601- | UAUUAACCAGAAGAAAACAA | 245 | 5246-5268 | 27573599- |
| 1446252.1 | AUA | | | 27573621 | GGA | | | 27573621 |
| AD- | UUGUUUUCUUCUGGUUAA | 156 | 5245-5265 | 27573602- | UGAUUAACCAGAAGAAAACA | 246 | 5245-5267 | 27573600- |
| 1446253.1 | UCA | | | 27573622 | AGG | | | 27573622 |
| AD- | UGUUUUCUUCUGGUUAAU | 157 | 5244-5264 | 27573603- | UAGAUUAACCAGAAGAAAAC | 247 | 5244-5266 | 27573601- |
| 1446254.1 | CUA | | | 27573623 | AAG | | | 27573623 |
| AD- | GUUUUCUUCUGGUUAAUC | 158 | 5243-5263 | 27573604- | UAAGAUUAACCAGAAGAAAA | 248 | 5243-5265 | 27573602- |
| 1446255.1 | UUA | | | 27573624 | CAA | | | 27573624 |
| AD- | UUCUUCUGGUUAAUCUUU | 159 | 5240-5260 | 27573607- | UAUAAAGAUUAACCAGAAGA | 249 | 5240-5262 | 27573605- |
| 1446256.1 | AUA | | | 27573627 | AAA | | | 27573627 |
| AD- | UCUUCUGGUUAAUCUUUA | 160 | 5239-5259 | 27573608- | UGAUAAAGAUUAACCAGAAG | 250 | 5239-5261 | 27573606- |
| 1446257.1 | UCA | | | 27573628 | AAA | | | 27573628 |
| AD- | CUUCUGGUUAAUCUUUAU | 161 | 5238-5258 | 27573609- | UUGAUAAAGAUUAACCAGAA | 251 | 5238-5260 | 27573607- |
| 1446258.1 | CAA | | | 27573629 | GAA | | | 27573629 |
| AD- | UUCUGGUUAAUCUUUAUC | 162 | 5237-5257 | 27573610- | UCUGAUAAAGAUUAACCAGA | 252 | 5237-5259 | 27573608- |
| 1446259.1 | AGA | | | 27573630 | AGA | | | 27573630 |
| AD- | UCUGGUUAAUCUUUAUCA | 163 | 5236-5256 | 27573611- | UCCUGAUAAAGAUUAACCAG | 253 | 5236-5258 | 27573609- |
| 1446260.1 | GGA | | | 27573631 | AAG | | | 27573631 |
| AD- | CUGGUUAAUCUUUAUCAG | 164 | 5235-5255 | 27573612- | UACCUGAUAAAGAUUAACCA | 254 | 5235-5257 | 27573610- |
| 1446261.1 | GUA | | | 27573632 | GAA | | | 27573632 |
| AD- | UGGUUAAUCUUUAUCAGG | 165 | 5234-5254 | 27573613- | UGACCUGAUAAAGAUUAACC | 255 | 5234-5256 | 27573611- |
| 1446262.1 | UCA | | | 27573633 | AGA | | | 27573633 |
| AD- | GUUAAUCUUUAUCAGGUC | 166 | 5232-5252 | 27573615- | UAAGACCUGAUAAAGAUUAA | 256 | 5232-5254 | 27573613- |
| 1446263.1 | UUA | | | 27573635 | CCA | | | 27573635 |
| AD- | UUAAUCUUUAUCAGGUCU | 167 | 5231-5251 | 27573616- | UAAAGACCUGAUAAAGAUUA | 257 | 5231-5253 | 27573614- |
| 1446264.1 | UUA | | | 27573636 | ACC | | | 27573636 |
| AD- | AAUCUUUAUCAGGUCUUU | 168 | 5229-5249 | 27573618- | UGAAAAGACCUGAUAAAGAU | 258 | 5229-5251 | 27573616- |
| 1446265.1 | UCA | | | 27573638 | UAA | | | 27573638 |
| AD- | AUCUUUAUCAGGUCUUUU | 169 | 5228-5248 | 27573619- | UAGAAAAGACCUGAUAAAGA | 259 | 5228-5250 | 27573617- |
| 1446266.1 | CUA | | | 27573639 | UUA | | | 27573639 |
| AD- | UCUUUAUCAGGUCUUUUC | 170 | 5227-5247 | 27573620- | UAAGAAAAGACCUGAUAAAG | 260 | 5227-5249 | 27573618- |
| 1446267.1 | UUA | | | 27573640 | AUU | | | 27573640 |
| AD- | CUUUAUCAGGUCUUUUCU | 171 | 5226-5246 | 27573621- | UCAAGAAAAGACCUGAUAAA | 261 | 5226-5248 | 27573619- |
| 1446268.1 | UGA | | | 27573641 | GAU | | | 27573641 |
| AD- | UUUAUCAGGUCUUUUCUU | 172 | 5225-5245 | 27573622- | UACAAGAAAAGACCUGAUAA | 262 | 5225-5247 | 27573620- |
| 1446269.1 | GUA | | | 27573642 | AGA | | | 27573642 |
| AD- | UUAUCAGGUCUUUUCUUG | 173 | 5224-5244 | 27573623- | UAACAAGAAAAGACCUGAUA | 263 | 5224-5246 | 27573621- |
| 1446270.1 | UUA | | | 27573643 | AAG | | | 27573643 |
| AD- | UAUCAGGUCUUUUCUUGU | 174 | 5223-5243 | 27573624- | UGAACAAGAAAAGACCUGAU | 1412 | 5223-5245 | 27573622- |
| 1446271.1 | UCA | | | 27573644 | AAA | | | 27573644 |
| AD- | AUCAGGUCUUUUCUUGUU | 175 | 5222-5242 | 27573625- | UUGAACAAGAAAAGACCUGA | 265 | 5222-5244 | 27573623- |
| 1446272.1 | CAA | | | 27573645 | UAA | | | 27573645 |
| AD- | UCAGGUCUUUUCUUGUUC | 176 | 5221-5241 | 27573626- | UGUGAACAAGAAAAGACCUG | 266 | 5221-5243 | 27573624- |
| 1446273.1 | ACA | | | 27573646 | AUA | | | 27573646 |
| AD- | CAGGUCUUUUCUUGUUCA | 177 | 5220-5240 | 27573627- | UGGUGAACAAGAAAAGACCU | 267 | 5220-5242 | 27573625- |
| 1446274.1 | CCA | | | 27573647 | GAU | | | 27573647 |
| AD- | GGUCUUUUCUUGUUCACC | 178 | 5218-5238 | 27573629- | UAGGGUGAACAAGAAAAGAC | 268 | 5218-5240 | 27573627- |
| 1446275.1 | CUA | | | 27573649 | CUG | | | 27573649 |
| AD- | UCUUUUCUUGUUCACCCUC | 179 | 5216-5236 | 27573631- | UUGAGGGUGAACAAGAAAAG | 269 | 5216-5238 | 27573629- |
| 1446276.1 | AA | | | 27573651 | ACC | | | 27573651 |
| AD- | CUUUUCUUGUUCACCCUCA | 180 | 5215-5235 | 27573632- | UCUGAGGGUGAACAAGAAAA | 270 | 5215-5237 | 27573630- |
| 1446277.1 | GA | | | 27573652 | GAC | | | 27573652 |
| AD- | UUUUCUUGUUCACCCUCA | 181 | 5214-5234 | 27573633- | UGCUGAGGGUGAACAAGAAA | 271 | 5214-5236 | 27573631- |
| 1446278.1 | GCA | | | 27573653 | AGA | | | 27573653 |
| AD- | UUCUUGUUCACCCUCAGCG | 182 | 5212-5232 | 27573635- | UUCGCUGAGGGUGAACAAGA | 272 | 5212-5234 | 27573633- |
| 1446279.1 | AA | | | 27573655 | AAA | | | 27573655 |
| AD- | CCUCAGCGAGUACUGUGA | 183 | 5201-5221 | 27573646- | UUCUCACAGUACUCGCUGAG | 273 | 5201-5223 | 27573644- |
| 1446280.1 | GAA | | | 27573666 | GGU | | | 27573666 |
| AD- | UCAGCGAGUACUGUGAGA | 184 | 5199-5219 | 27573648- | UGCUCUCACAGUACUCGCUG | 274 | 5199-5221 | 27573646- |
| 1446281.1 | GCA | | | 27573668 | AGG | | | 27573668 |
| AD- | CAGCGAGUACUGUGAGAG | 185 | 5198-5218 | 27573649- | UUGCUCTCACAGUACUCGCUG | 275 | 5198-5220 | 27573647- |
| 1446282.1 | CAA | | | 27573669 | AG | | | 27573669 |
| AD- | AGCGAGUACUGUGAGAGC | 186 | 5197-5217 | 27573650- | UUUGCUCUCACAGUACUCGC | 276 | 5197-5219 | 27573648- |
| 1446283.1 | AAA | | | 27573670 | UGA | | | 27573670 |
| AD- | GCGAGUACUGUGAGAGCA | 187 | 5196-5216 | 27573651- | UCUUGCTCUCACAGUACUCGC | 277 | 5196-5218 | 27573649- |
| 1446284.1 | AGA | | | 27573671 | UG | | | 27573671 |
| AD- | CGAGUACUGUGAGAGCAA | 188 | 5195-5215 | 27573652- | UACUUGCUCUCACAGUACUC | 278 | 5195-5217 | 27573650- |
| 1446285.1 | GUA | | | 27573672 | GCU | | | 27573672 |
| AD- | GAGUACUGUGAGAGCAAG | 189 | 5194-5214 | 27573653- | UUACUUGCUCUCACAGUACU | 279 | 5194-5216 | 27573651- |
| 1446286.1 | UAA | | | 27573673 | CGC | | | 27573673 |
| AD- | AGUACUGUGAGAGCAAGU | 190 | 5193-5213 | 27573654- | UCUACUUGCUCUCACAGUAC | 280 | 5193-5215 | 27573652- |
| 1446287.1 | AGA | | | 27573674 | UCG | | | 27573674 |
| AD- | UACUGUGAGAGCAAGUAG | 191 | 5191-5211 | 27573656- | UCACUACUUGCUCUCACAGU | 281 | 5191-5213 | 27573654- |
| 1446288.1 | UGA | | | 27573676 | ACU | | | 27573676 |
| AD- | AAAACAAAAACACACACC | 192 | 5155-5175 | 27573692- | UGAGGUGUGUGUUUUUGUUU | 282 | 5155-5177 | 27573690- |
| 1446289.1 | UCA | | | 27573712 | UUC | | | 27573712 |
| AD- | ACACCUCCUAAACCCACAC | 193 | 5142-5162 | 27573705- | UGGUGUGGGUUUAGGAGGUG | 283 | 5142-5164 | 27573703- |
| 1446290.1 | CA | | | 27573725 | UGU | | | 27573725 |
| AD- | ACCUCCUAAACCCACACCU | 194 | 5140-5160 | 27573707- | UCAGGUGUGGGUUUAGGAGG | 284 | 5140-5162 | 27573705- |
| 1446291.1 | GA | | | 27573727 | UGU | | | 27573727 |
| AD- | CUCCUAAACCCACACCUGC | 195 | 5138-5158 | 27573709- | UAGCAGGUGUGGGUUUAGGA | 285 | 5138-5160 | 27573707- |
| 1446292.1 | UA | | | 27573729 | GGU | | | 27573729 |
| AD- | CCACACCUGCUCUUGCUAG | 196 | 5129-5149 | 27573718- | UUCUAGCAAGAGCAGGUGUG | 286 | 5129-5151 | 27573716- |
| 1446293.1 | AA | | | 27573738 | GGU | | | 27573738 |
| AD- | CACACCUGCUCUUGCUAGA | 197 | 5128-5148 | 27573719- | UGUCUAGCAAGAGCAGGUGU | 287 | 5128-5150 | 27573717- |
| 1446294.1 | CA | | | 27573739 | GGG | | | 27573739 |
| AD- | ACACCUGCUCUUGCUAGAC | 198 | 5127-5147 | 27573720- | UGGUCUAGCAAGAGCAGGUG | 288 | 5127-5149 | 27573718- |
| 1446295.1 | CA | | | 27573740 | UGG | | | 27573740 |

TABLE 3
Modified Sense and Antisense Strand Sequences of dsRNA Agents Targeting the Antisense Strand of Intron la of Human C9orf72

| | | SEQ | | SEQ | | SEQ |
|---|---|---|---|---|---|---|
| Duplex | | ID | | ID | | ID |
| Name | Sense Sequence 5′ to 3′ | NO: | Antisense Sequence 5′ to 3′ | NO: | mRNA Target Sequence 5′ to 3′ | NO: |
| AD- | cscsgag(Ghd)CfuCfCfCfuuuucucgsasa | 289 | VPusUfscgaGfaAfAfagggAfgCfcucggsgsu | 379 | ACCCGAGGCUCCCUUUUCUCGAG | 469 |
| 1446206.1 | | | | | | |
| AD- | asgsgca(Ahd)UfuCfCfAfccagucgcsusa | 290 | VPusAfsgcgAfcUfGfguggAfaUfugccusgsc | 380 | GCAGGCAAUUCCACCAGUCGCUA | 470 |
| 1446207.1 | | | | | | |
| AD- | csascca(Ghd)UfcGfCfUfagaggcgasasa | 291 | VPusUfsucgc(C2p)ucuagcGfaCfuggugsgsa | 381 | UCCACCAGUCGCUAGAGGCGAAA | 471 |
| 1446208.1 | | | | | | |
| AD- | ascscag(Uhd)CfgCfUfAfgaggcgaasasa | 292 | VPusUfsuucg(C2p)cucuagCfgAfcuggusgsg | 382 | CCACCAGUCGCUAGAGGCGAAAG | 472 |
| 1446209.1 | | | | | | |
| AD- | csasccc(Ahd)GfcUfUfCfggucagagsasa | 293 | VPusUfscucu(G2p)accgaaGfcUfgggugsusc | 383 | GACACCCAGCUUCGGUCAGAGAA | 473 |
| 1446210.1 | | | | | | |
| AD- | cscscag(Chd)UfuCfGfGfucagagaasasa | 294 | VPusUfsuucu(C2p)ugaccgAfaGfcugggsusg | 384 | CACCCAGCUUCGGUCAGAGAAAU | 474 |
| 1446211.1 | | | | | | |
| AD- | csasgcu(Uhd)CfgGfUfCfagagaaausgsa | 295 | VPusCfsauuu(C2p)ucugacCfgAfagcugsgsg | 385 | CCCAGCUUCGGUCAGAGAAAUGA | 475 |
| 1446212.1 | | | | | | |
| AD- | gscsuuc(Ghd)GfuCfAfGfagaaaugasgsa | 296 | VPusCfsucaUfuUfCfucugAfcCfgaagcsusg | 386 | CAGCUUCGGUCAGAGAAAUGAGA | 476 |
| 1446213.1 | | | | | | |
| AD- | uscsggu(Chd)AfgAfGfAfaaugagagsgsa | 297 | VPusCfscucu(C2p)auuucuCfuGfaccgasasg | 387 | CUUCGGUCAGAGAAAUGAGAGGG | 477 |
| 1446214.1 | | | | | | |
| AD- | gsgsuca(Ghd)AfgAfAfAfugagagggsasa | 298 | VPusUfscccu(C2p)ucauuuCfuCfugaccsgsa | 388 | UCGGUCAGAGAAAUGAGAGGGAA | 478 |
| 1446215.1 | | | | | | |
| AD- | uscsaga(Ghd)AfaAfUfGfagagggaasasa | 299 | VPusUfsuucc(C2p)ucucauUfuCfucugascsc | 389 | GGUCAGAGAAAUGAGAGGGAAAG | 479 |
| 1446216.1 | | | | | | |
| AD- | asgsagg(Ghd)AfaAfGfUfaaaaaugcsgsa | 300 | VPusCfsgcaUfuUfUfuacuUfuCfccucuscsa | 390 | UGAGAGGGAAAGUAAAAAUGCGU | 480 |
| 1446217.1 | | | | | | |
| AD- | gsasggg(Ahd)AfaGfUfAfaaaaugcgsusa | 301 | VPusAfscgcAfuUfUfuuacUfuUfcccucsusc | 391 | GAGAGGGAAAGUAAAAAUGCGUC | 481 |
| 1446218.1 | | | | | | |
| AD- | asgsgga(Ahd)AfgUfAfAfaaaugcguscsa | 302 | VPusGfsacgCfaUfUfuuuaCfuUfucccuscsu | 392 | AGAGGGAAAGUAAAAAUGCGUCG | 482 |
| 1446219.1 | | | | | | |
| AD- | gsgsgaa(Ahd)GfuAfAfAfaaugcgucsgsa | 303 | VPusCfsgacGfcAfUfuuuuAfcUfuucccsusc | 393 | GAGGGAAAGUAAAAAUGCGUCGA | 483 |
| 1446220.1 | | | | | | |
| AD- | gsgsaaa(Ghd)UfaAfAfAfaugcgucgsasa | 304 | VPusUfscgaCfgCfAfuuuuUfaCfuuuccscsu | 394 | AGGGAAAGUAAAAAUGCGUCGAG | 484 |
| 1446221.1 | | | | | | |
| AD- | gsasaag(Uhd)AfaAfAfAfugcgucgasgsa | 305 | VPusCfsucgAfcGfCfauuuUfuAfcuuucscsc | 395 | GGGAAAGUAAAAAUGCGUCGAGC | 485 |
| 1446222.1 | | | | | | |
| AD- | asasagu(Ahd)AfaAfAfUfgcgucgagscsa | 306 | VPusGfscucGfaCfGfcauuUfuUfacuuuscsc | 396 | GGAAAGUAAAAAUGCGUCGAGCU | 486 |
| 1446223.1 | | | | | | |
| AD- | asasgua(Ahd)AfaAfUfGfcgucgagcsusa | 307 | VPusAfsgcuCfgAfCfgcauUfuUfuacuususc | 397 | GAAAGUAAAAAUGCGUCGAGCUC | 487 |
| 1446224.1 | | | | | | |
| AD- | asgsuaa(Ahd)AfaUfGfCfgucgagcuscsa | 308 | VPusGfsagcu(C2p)gacgcaUfuUfuuacususu | 398 | AAAGUAAAAAUGCGUCGAGCUCU | 488 |
| 1446225.1 | | | | | | |
| AD- | csgsacu(Chd)CfuGfAfGfuuccagagscsa | 309 | VPusGfscucu(G2p)gaacucAfgGfagucgscsg | 399 | CGCGACUCCUGAGUUCCAGAGCU | 489 |
| 1446226.1 | | | | | | |
| AD- | gsascuc(Chd)UfgAfGfUfuccagagcsusa | 310 | VPusAfsgcuc(Tgn)ggaacuCfaGfgagucsgsc | 400 | GCGACUCCUGAGUUCCAGAGCUU | 490 |
| 1446227.1 | | | | | | |
| AD- | ascsucc(Uhd)GfaGfUfUfccagagcususa | 311 | VPusAfsagcu(C2p)uggaacUfcAfggaguscsg | 401 | CGACUCCUGAGUUCCAGAGCUUG | 491 |
| 1446228.1 | | | | | | |
| AD- | csusccu(Ghd)AfgUfUfCfcagagcuusgsa | 312 | VPusCfsaagc(Tgn)cuggaaCfuCfaggagsusc | 402 | GACUCCUGAGUUCCAGAGCUUGC | 492 |
| 1446229.1 | | | | | | |
| AD- | uscscug(Ahd)GfuUfCfCfagagcuugscsa | 313 | VPusGfscaag(C2p)ucuggaAfcUfcaggasgsu | 403 | ACUCCUGAGUUCCAGAGCUUGCU | 493 |
| 1446230.1 | | | | | | |
| AD- | cscsuga(Ghd)UfuCfCfAfgagcuugcsusa | 314 | VPusAfsgcaa(G2p)cucuggAfaCfucaggsasg | 404 | CUCCUGAGUUCCAGAGCUUGCUA | 494 |
| 1446231.1 | | | | | | |
| AD- | gsasguu(Chd)CfaGfAfGfcuugcuacsasa | 315 | VPusUfsguag(C2p)aagcucUfgGfaacucsasg | 405 | CUGAGUUCCAGAGCUUGCUACAG | 495 |
| 1446232.1 | | | | | | |
| AD- | asgsuuc(Chd)AfgAfGfCfuugcuacasgsa | 316 | VPusCfsugua(G2p)caagcuCfuGfgaacuscsa | 406 | UGAGUUCCAGAGCUUGCUACAGG | 496 |
| 1446233.1 | | | | | | |
| AD- | gsusucc(Ahd)GfaGfCfUfugcuacagsgsa | 317 | VPusCfscugUfaGfCfaagcUfcUfggaacsusc | 407 | GAGUUCCAGAGCUUGCUACAGGC | 497 |
| 1446234.1 | | | | | | |
| AD- | ususcca(Ghd)AfgCfUfUfgcuacaggscsa | 318 | VPusGfsccug(Tgn)agcaagCfuCfuggaascsu | 408 | AGUUCCAGAGCUUGCUACAGGCU | 498 |
| 1446235.1 | | | | | | |
| AD- | uscscag(Ahd)GfcUfUfGfcuacaggcsusa | 319 | VPusAfsgccu(G2p)uagcaaGfcUfcuggasasc | 409 | GUUCCAGAGCUUGCUACAGGCUG | 499 |
| 1446236.1 | | | | | | |
| AD- | cscsaga(Ghd)CfuUfGfCfuacaggcusgsa | 320 | VPusCfsagcc(Tgn)guagcaAfgCfucuggsasa | 410 | UUCCAGAGCUUGCUACAGGCUGC | 500 |
| 1446237.1 | | | | | | |
| AD- | csasgag(Chd)UfuGfCfUfacaggcugscsa | 321 | VPusGfscagc(C2p)uguagcAfaGfcucugsgsa | 411 | UCCAGAGCUUGCUACAGGCUGCG | 501 |
| 1446238.1 | | | | | | |
| AD- | usascag(Ghd)CfuGfCfGfguuguuucscsa | 322 | VPusGfsgaaa(C2p)aaccgcAfgCfcuguasgsc | 412 | GCUACAGGCUGCGGUUGUUUCCC | 502 |
| 1446239.1 | | | | | | |
| AD- | gscsugc(Ghd)GfuUfGfUfuucccuccsusa | 323 | VPusAfsggag(G2p)gaaacaAfcCfgcagcscsu | 413 | AGGCUGCGGUUGUUUCCCUCCUU | 503 |
| 1446240.1 | | | | | | |
| AD- | csusgcg(Ghd)UfuGfUfUfucccuccususa | 324 | VPusAfsagga(G2p)ggaaacAfaCfcgcagscsc | 414 | GGCUGCGGUUGUUUCCCUCCUUG | 504 |
| 1446241.1 | | | | | | |
| AD- | gscsggu(Uhd)GfuUfUfCfccuccuugsusa | 325 | VPusAfscaag(G2p)agggaaAfcAfaccgcsasg | 415 | CUGCGGUUGUUUCCCUCCUUGUU | 505 |
| 1446242.1 | | | | | | |
| AD- | csgsguu(Ghd)UfuUfCfCfcuccuugususa | 326 | VPusAfsacaAfgGfAfgggaAfaCfaaccgscsa | 416 | UGCGGUUGUUUCCCUCCUUGUUU | 506 |
| 1446243.1 | | | | | | |
| AD- | gsusugu(Uhd)UfcCfCfUfccuuguuususa | 327 | VPusAfsaaaCfaAfGfgaggGfaAfacaacscsg | 417 | CGGUUGUUUCCCUCCUUGUUUUC | 507 |
| 1446244.1 | | | | | | |
| AD- | ususguu(Uhd)CfcCfUfCfcuuguuuuscsa | 328 | VPusGfsaaaAfcAfAfggagGfgAfaacaascsc | 418 | GGUUGUUUCCCUCCUUGUUUUCU | 508 |
| 1446245.1 | | | | | | |
| AD- | ususccc(Uhd)CfcUfUfGfuuuucuucsusa | 329 | VPusAfsgaaGfaAfAfacaaGfgAfgggaasasc | 419 | GUUUCCCUCCUUGUUUUCUUCUG | 509 |
| 1446246.1 | | | | | | |
| AD- | uscsccu(Chd)CfuUfGfUfuuucuucusgsa | 330 | VPusCfsagaAfgAfAfaacaAfgGfagggasasa | 420 | UUUCCCUCCUUGUUUUCUUCUGG | 510 |
| 1446247.1 | | | | | | |
| AD- | cscscuc(Chd)UfuGfUfUfuucuucugsgsa | 331 | VPusCfscagAfaGfAfaaacAfaGfgagggsasa | 421 | UUCCCUCCUUGUUUUCUUCUGGU | 511 |
| 1446248.1 | | | | | | |
| AD- | cscsucc(Uhd)UfgUfUfUfucuucuggsusa | 332 | VPusAfsccaGfaAfGfaaaaCfaAfggaggsgsa | 422 | UCCCUCCUUGUUUUCUUCUGGUU | 512 |
| 1446249.1 | | | | | | |
| AD- | csusccu(Uhd)GfuUfUfUfcuucuggususa | 333 | VPusAfsaccAfgAfAfgaaaAfcAfaggagsgsg | 423 | CCCUCCUUGUUUUCUUCUGGUUA | 513 |
| 1446250.1 | | | | | | |
| AD- | cscsuug(Uhd)UfuUfCfUfucugguuasasa | 334 | VPusUfsuaac(C2p)agaagaAfaAfcaaggsasg | 424 | CUCCUUGUUUUCUUCUGGUUAAU | 514 |
| 1446251.1 | | | | | | |
| AD- | csusugu(Uhd)UfuCfUfUfcugguuaasusa | 335 | VPusAfsuuaAfcCfAfgaagAfaAfacaagsgsa | 425 | UCCUUGUUUUCUUCUGGUUAAUC | 515 |
| 1446252.1 | | | | | | |
| AD- | ususguu(Uhd)UfcUfUfCfugguuaauscsa | 336 | VPusGfsauuAfaCfCfagaaGfaAfaacaasgsg | 426 | CCUUGUUUUCUUCUGGUUAAUCU | 516 |
| 1446253.1 | | | | | | |
| AD- | usgsuuu(Uhd)CfuUfCfUfgguuaaucsusa | 337 | VPusAfsgauUfaAfCfcagaAfgAfaaacasasg | 427 | CUUGUUUUCUUCUGGUUAAUCUU | 517 |
| 1446254.1 | | | | | | |
| AD- | gsusuuu(Chd)UfuCfUfGfguuaaucususa | 338 | VPusAfsagaUfuAfAfccagAfaGfaaaacsasa | 428 | UUGUUUUCUUCUGGUUAAUCUUU | 518 |
| 1446255.1 | | | | | | |
| AD- | ususcuu(Chd)UfgGfUfUfaaucuuuasusa | 339 | VPusAfsuaaAfgAfUfuaacCfaGfaagaasasa | 429 | UUUUCUUCUGGUUAAUCUUUAUC | 519 |
| 1446256.1 | | | | | | |
| AD- | uscsuuc(Uhd)GfgUfUfAfaucuuuauscsa | 340 | VPusGfsauaAfaGfAfuuaaCfcAfgaagasasa | 430 | UUUCUUCUGGUUAAUCUUUAUCA | 520 |
| 1446257.1 | | | | | | |
| AD- | csusucu(Ghd)GfuUfAfAfucuuuaucsasa | 341 | VPusUfsgauAfaAfGfauuaAfcCfagaagsasa | 431 | UUCUUCUGGUUAAUCUUUAUCAG | 521 |
| 1446258.1 | | | | | | |
| AD- | ususcug(Ghd)UfuAfAfUfcuuuaucasgsa | 342 | VPusCfsugaUfaAfAfgauuAfaCfcagaasgsa | 432 | UCUUCUGGUUAAUCUUUAUCAGG | 522 |
| 1446259.1 | | | | | | |
| AD- | uscsugg(Uhd)UfaAfUfCfuuuaucagsgsa | 343 | VPusCfscugAfuAfAfagauUfaAfccagasasg | 433 | CUUCUGGUUAAUCUUUAUCAGGU | 523 |
| 1446260.1 | | | | | | |
| AD- | csusggu(Uhd)AfaUfCfUfuuaucaggsusa | 344 | VPusAfsccug(Agn)uaaagaUfuAfaccagsasa | 434 | UUCUGGUUAAUCUUUAUCAGGUC | 524 |
| 1446261.1 | | | | | | |
| AD- | usgsguu(Ahd)AfuCfUfUfuaucagguscsa | 345 | VPusGfsaccu(G2p)auaaagAfuUfaaccasgsa | 435 | UCUGGUUAAUCUUUAUCAGGUCU | 525 |
| 1446262.1 | | | | | | |
| AD- | gsusuaa(Uhd)CfuUfUfAfucaggucususa | 346 | VPusAfsagac(C2p)ugauaaAfgAfuuaacscsa | 436 | UGGUUAAUCUUUAUCAGGUCUUU | 526 |
| 1446263.1 | | | | | | |
| AD- | ususaau(Chd)UfuUfAfUfcaggucuususa | 347 | VPusAfsaaga(C2p)cugauaAfaGfauuaascsc | 437 | GGUUAAUCUUUAUCAGGUCUUUU | 527 |
| 1446264.1 | | | | | | |
| AD- | asasucu(Uhd)UfaUfCfAfggucuuuuscsa | 348 | VPusGfsaaaAfgAfCfcugaUfaAfagauusasa | 438 | UUAAUCUUUAUCAGGUCUUUUCU | 528 |
| 1446265.1 | | | | | | |
| AD- | asuscuu(Uhd)AfuCfAfGfgucuuuucsusa | 349 | VPusAfsgaaAfaGfAfccugAfuAfaagaususa | 439 | UAAUCUUUAUCAGGUCUUUUCUU | 529 |
| 1446266.1 | | | | | | |
| AD- | uscsuuu(Ahd)UfcAfGfGfucuuuucususa | 350 | VPusAfsagaAfaAfGfaccuGfaUfaaagasusu | 440 | AAUCUUUAUCAGGUCUUUUCUUG | 530 |
| 1446267.1 | | | | | | |
| AD- | csusuua(Uhd)CfaGfGfUfcuuuucuusgsa | 351 | VPusCfsaagAfaAfAfgaccUfgAfuaaagsasu | 441 | AUCUUUAUCAGGUCUUUUCUUGU | 531 |
| 1446268.1 | | | | | | |
| AD- | ususuau(Chd)AfgGfUfCfuuuucuugsusa | 352 | VPusAfscaaGfaAfAfagacCfuGfauaaasgsa | 442 | UCUUUAUCAGGUCUUUUCUUGUU | 532 |
| 1446269.1 | | | | | | |
| AD- | ususauc(Ahd)GfgUfCfUfuuucuugususa | 353 | VPusAfsacaAfgAfAfaagaCfcUfgauaasasg | 443 | CUUUAUCAGGUCUUUUCUUGUUC | 533 |
| 1446270.1 | | | | | | |
| AD- | usasuca(Ghd)GfuCfUfUfuucuuguuscsa | 354 | VPusGfsaacAfaGfAfaaagAfcCfugauasasa | 444 | UUUAUCAGGUCUUUUCUUGUUCA | 534 |
| 1446271.1 | | | | | | |
| AD- | asuscag(Ghd)UfcUfUfUfucuuguucsasa | 355 | VPusUfsgaaCfaAfGfaaaaGfaCfcugausasa | 445 | UUAUCAGGUCUUUUCUUGUUCAC | 535 |
| 1446272.1 | | | | | | |
| AD- | uscsagg(Uhd)CfuUfUfUfcuuguucascsa | 356 | VPusGfsugaa(C2p)aagaaaAfgAfccugasusa | 446 | UAUCAGGUCUUUUCUUGUUCACC | 536 |
| 1446273.1 | | | | | | |
| AD- | csasggu(Chd)UfuUfUfCfuuguucacscsa | 357 | VPusGfsguga(Agn)caagaaAfaGfaccugsasu | 447 | AUCAGGUCUUUUCUUGUUCACCC | 537 |
| 1446274.1 | | | | | | |
| AD- | gsgsucu(Uhd)UfuCfUfUfguucacccsusa | 358 | VPusAfsgggu(G2p)aacaagAfaAfagaccsusg | 448 | CAGGUCUUUUCUUGUUCACCCUC | 538 |
| 1446275.1 | | | | | | |
| AD- | uscsuuu(Uhd)CfuUfGfUfucacccucsasa | 359 | VPusUfsgagg(G2p)ugaacaAfgAfaaagascsc | 449 | GGUCUUUUCUUGUUCACCCUCAG | 539 |
| 1446276.1 | | | | | | |
| AD- | csusuuu(Chd)UfuGfUfUfcacccucasgsa | 360 | VPusCfsugag(G2p)gugaacAfaGfaaaagsasc | 450 | GUCUUUUCUUGUUCACCCUCAGC | 540 |
| 1446277.1 | | | | | | |
| AD- | ususuuc(Uhd)UfgUfUfCfacccucagscsa | 361 | VPusGfscuga(G2p)ggugaaCfaAfgaaaasgsa | 451 | UCUUUUCUUGUUCACCCUCAGCG | 541 |
| 1446278.1 | | | | | | |
| AD- | ususcuu(Ghd)UfuCfAfCfccucagcgsasa | 362 | VPusUfscgcu(G2p)agggugAfaCfaagaasasa | 452 | UUUUCUUGUUCACCCUCAGCGAG | 542 |
| 1446279.1 | | | | | | |
| AD- | cscsuca(Ghd)CfgAfGfUfacugugagsasa | 363 | VPusUfscuca(C2p)aguacuCfgCfugaggsgsu | 453 | ACCCUCAGCGAGUACUGUGAGAG | 543 |
| 1446280.1 | | | | | | |
| AD- | uscsagc(Ghd)AfgUfAfCfugugagagscsa | 364 | VPusGfscucu(C2p)acaguaCfuCfgcugasgsg | 454 | CCUCAGCGAGUACUGUGAGAGCA | 544 |
| 1446281.1 | | | | | | |
| AD- | csasgcg(Ahd)GfuAfCfUfgugagagcsasa | 365 | VPusUfsgcuc(Tgn)cacaguAfcUfcgcugsasg | 455 | CUCAGCGAGUACUGUGAGAGCAA | 545 |
| 1446282.1 | | | | | | |
| AD- | asgscga(Ghd)UfaCfUfGfugagagcasasa | 366 | VPusUfsugcu(C2p)ucacagUfaCfucgcusgsa | 456 | UCAGCGAGUACUGUGAGAGCAAG | 546 |
| 1446283.1 | | | | | | |
| AD- | gscsgag(Uhd)AfcUfGfUfgagagcaasgsa | 367 | VPusCfsuugc(Tgn)cucacaGfuAfcucgcsusg | 457 | CAGCGAGUACUGUGAGAGCAAGU | 547 |
| 1446284.1 | | | | | | |
| AD- | csgsagu(Ahd)CfuGfUfGfagagcaagsusa | 368 | VPusAfscuug(C2p)ucucacAfgUfacucgscsu | 458 | AGCGAGUACUGUGAGAGCAAGUA | 548 |
| 1446285.1 | | | | | | |
| AD- | gsasgua(Chd)UfgUfGfAfgagcaagusasa | 369 | VPusUfsacuu(G2p)cucucaCfaGfuacucsgsc | 459 | GCGAGUACUGUGAGAGCAAGUAG | 549 |
| 1446286.1 | | | | | | |
| AD- | asgsuac(Uhd)GfuGfAfGfagcaaguasgsa | 370 | VPusCfsuacUfuGfCfucucAfcAfguacuscsg | 460 | CGAGUACUGUGAGAGCAAGUAGU | 550 |
| 1446287.1 | | | | | | |
| AD- | usascug(Uhd)GfaGfAfGfcaaguagusgsa | 371 | VPusCfsacuAfcUfUfgcucUfcAfcaguascsu | 461 | AGUACUGUGAGAGCAAGUAGUGG | 551 |
| 1446288.1 | | | | | | |
| AD- | asasaac(Ahd)AfaAfAfCfacacaccuscsa | 372 | VPusGfsaggu(G2p)uguguuUfuUfguuuususc | 462 | GAAAAACAAAAACACACACCUCC | 552 |
| 1446289.1 | | | | | | |
| AD- | ascsacc(Uhd)CfcUfAfAfacccacacscsa | 373 | VPusGfsgugu(G2p)gguuuaGfgAfggugusgsu | 463 | ACACACCUCCUAAACCCACACCU | 553 |
| 1446290.1 | | | | | | |
| AD- | ascscuc(Chd)UfaAfAfCfccacaccusgsa | 374 | VPusCfsaggu(G2p)uggguuUfaGfgaggusgsu | 464 | ACACCUCCUAAACCCACACCUGC | 554 |
| 1446291.1 | | | | | | |
| AD- | csusccu(Ahd)AfaCfCfCfacaccugcsusa | 375 | VPusAfsgcag(G2p)ugugggUfuUfaggagsgsu | 465 | ACCUCCUAAACCCACACCUGCUC | 555 |
| 1446292.1 | | | | | | |
| AD- | cscsaca(Chd)CfuGfCfUfcuugcuagsasa | 376 | VPusUfscuag(C2p)aagagcAfgGfuguggsgsu | 466 | ACCCACACCUGCUCUUGCUAGAC | 556 |
| 1446293.1 | | | | | | |
| AD- | csascac(Chd)UfgCfUfCfuugcuagascsa | 377 | VPusGfsucua(G2p)caagagCfaGfgugugsgsg | 467 | CCCACACCUGCUCUUGCUAGACC | 557 |
| 1446294.1 | | | | | | |
| AD- | ascsacc(Uhd)GfcUfCfUfugcuagacscsa | 378 | VPusGfsgucu(Agn)gcaagaGfcAfggugusgsg | 468 | CCACACCUGCUCUUGCUAGACCC | 558 |
| 1446295.1 | | | | | | |

TABLE 4
Single Dose Screen of dsRNA Agents Targeting the Antisense
Strand of Intron 1a of Human C9orf72 in Cos-7 Cells

| | 10 nM % | | 1 nM % | | 0.1 nM % | |
|---|---|---|---|---|---|---|
| | Message* | | Message* | | Message* | |
| Duplex | Remaining | STDEV | Remaining | STDEV | Remaining | STDEV |
| AD-1446206.1 | 45 | 9 | 65 | 12 | 85 | 18 |
| AD-1446207.1 | 18 | 4 | 23 | 3 | 38 | 4 |
| AD-1446208.1 | 51 | 5 | 52 | 8 | 68 | 15 |
| AD-1446209.1 | 77 | 11 | 81 | 15 | 95 | 9 |
| AD-1446210.1 | 81 | 24 | 98 | 10 | 102 | 10 |
| AD-1446211.1 | 19 | 3 | 25 | 2 | 39 | 7 |
| AD-1446212.1 | 64 | 11 | 61 | 6 | 83 | 7 |
| AD-1446213.1 | 9 | 2 | 11 | 1 | 20 | 2 |
| AD-1446214.1 | 18 | 1 | 23 | 3 | 30 | 5 |
| AD-1446215.1 | 18 | 5 | 18 | 4 | 28 | 9 |
| AD-1446216.1 | 15 | 3 | 18 | 4 | 28 | 5 |
| AD-1446217.1 | 14 | 3 | 17 | 2 | 26 | 2 |
| AD-1446218.1 | 12 | 1 | 15 | 1 | 22 | 7 |
| AD-1446219.1 | 12 | 3 | 19 | 4 | 24 | 5 |
| AD-1446220.1 | 15 | 4 | 21 | 3 | 28 | 5 |
| AD-1446221.1 | 7 | 0 | 11 | 2 | 17 | 5 |
| AD-1446222.1 | 10 | 2 | 16 | 4 | 26 | 6 |
| AD-1446223.1 | 11 | 1 | 18 | 6 | 37 | 4 |
| AD-1446224.1 | 15 | 2 | 20 | 3 | 34 | 7 |
| AD-1446225.1 | 17 | 3 | 25 | 2 | 42 | 5 |
| AD-1446226.1 | 19 | 4 | 22 | 3 | 28 | 2 |
| AD-1446227.1 | 16 | 7 | 22 | 5 | 31 | 7 |
| AD-1446228.1 | 25 | 8 | 28 | 6 | 42 | 9 |
| AD-1446229.1 | 16 | 3 | 21 | 2 | 31 | 2 |
| AD-1446230.1 | 21 | 2 | 20 | 6 | 28 | 3 |
| AD-1446231.1 | 17 | 1 | 22 | 4 | 25 | 4 |
| AD-1446232.1 | 11 | 2 | 18 | 4 | 22 | 2 |
| AD-1446233.1 | 16 | 1 | 23 | 3 | 32 | 6 |
| AD-1446234.1 | 18 | 3 | 26 | 3 | 34 | 7 |
| AD-1446235.1 | 30 | 9 | 37 | 5 | 53 | 8 |
| AD-1446236.1 | 66 | 12 | 82 | 11 | 105 | 25 |
| AD-1446237.1 | 23 | 4 | 34 | 5 | 49 | 5 |
| AD-1446238.1 | 44 | 8 | 41 | 12 | 49 | 7 |
| AD-1446239.1 | 53 | 9 | 61 | 8 | 74 | 7 |
| AD-1446240.1 | 25 | 5 | 33 | 8 | 44 | 4 |
| AD-1446241.1 | 23 | 4 | 31 | 7 | 38 | 6 |
| AD-1446242.1 | 12 | 2 | 16 | 4 | 26 | 3 |
| AD-1446243.1 | 18 | 1 | 22 | 2 | 27 | 2 |
| AD-1446244.1 | 53 | 7 | 44 | 7 | 51 | 16 |
| AD-1446245.1 | 18 | 2 | 16 | 1 | 22 | 2 |
| AD-1446246.1 | 7 | 1 | 10 | 1 | 13 | 3 |
| AD-1446247.1 | 7 | 1 | 9 | 1 | 12 | 2 |
| AD-1446248.1 | 5 | 1 | 6 | 1 | 9 | 3 |
| AD-1446249.1 | 7 | 1 | 9 | 2 | 12 | 2 |
| AD-1446250.1 | 12 | 1 | 14 | 2 | 17 | 4 |
| AD-1446251.1 | 7 | 2 | 7 | 0 | 10 | 2 |
| AD-1446252.1 | 4 | 1 | 4 | 1 | 6 | 4 |
| AD-1446253.1 | 1 | 1 | 4 | 1 | 7 | 3 |
| AD-1446254.1 | 6 | 3 | 5 | 0 | 7 | 3 |
| AD-1446255.1 | 11 | 2 | 7 | 1 | 9 | 1 |
| AD-1446256.1 | 34 | 7 | 27 | 4 | 31 | 2 |
| AD-1446257.1 | 5 | 1 | 5 | 0 | 6 | 1 |
| AD-1446258.1 | 5 | 2 | 4 | 1 | 6 | 1 |
| AD-1446259.1 | 6 | 2 | 7 | 1 | 8 | 1 |
| AD-1446260.1 | 4 | 1 | 4 | 1 | 5 | 1 |
| AD-1446261.1 | 10 | 4 | 9 | 1 | 10 | 2 |
| AD-1446262.1 | 6 | 1 | 6 | 0 | 7 | 1 |
| AD-1446263.1 | 5 | 1 | 6 | 1 | 9 | 2 |
| AD-1446264.1 | 11 | 4 | 8 | 1 | 9 | 1 |
| AD-1446265.1 | 6 | 1 | 6 | 1 | 7 | 1 |
| AD-1446266.1 | 6 | 1 | 6 | 0 | 8 | 2 |
| AD-1446267.1 | 8 | 4 | 8 | 3 | 11 | 1 |
| AD-1446268.1 | 4 | 1 | 3 | 1 | 6 | 1 |
| AD-1446269.1 | 5 | 1 | 6 | 0 | 7 | 2 |
| AD-1446270.1 | 5 | 1 | 7 | 3 | 8 | 2 |
| AD-1446271.1 | 3 | 1 | 4 | 2 | 7 | 1 |
| AD-1446272.1 | 8 | 4 | 7 | 1 | 9 | 2 |
| AD-1446273.1 | 6 | 2 | 8 | 2 | 10 | 1 |
| AD-1446274.1 | 5 | 2 | 6 | 1 | 9 | 2 |
| AD-1446275.1 | 10 | 3 | 15 | 5 | 17 | 3 |
| AD-1446276.1 | 6 | 1 | 6 | 1 | 8 | 2 |
| AD-1446277.1 | 12 | 1 | 12 | 1 | 16 | 4 |
| AD-1446278.1 | 16 | 2 | 16 | 2 | 22 | 7 |
| AD-1446279.1 | 23 | 3 | 19 | 3 | 28 | 4 |
| AD-1446280.1 | 20 | 8 | 30 | 12 | 40 | 14 |
| AD-1446281.1 | 21 | 4 | 31 | 3 | 51 | 14 |
| AD-1446282.1 | 18 | 5 | 17 | 4 | 40 | 9 |
| AD-1446283.1 | 44 | 6 | 32 | 7 | 50 | 14 |
| AD-1446284.1 | 25 | 8 | 31 | 3 | 52 | 13 |
| AD-1446285.1 | 38 | 9 | 49 | 4 | 52 | 11 |
| AD-1446286.1 | 28 | 6 | 48 | 8 | 67 | 15 |
| AD-1446287.1 | 64 | 19 | 56 | 10 | 72 | 13 |
| AD-1446288.1 | 34 | 5 | 34 | 7 | 54 | 10 |
| AD-1446289.1 | 17 | 1 | 21 | 3 | 35 | 9 |
| AD-1446290.1 | 40 | 2 | 61 | 10 | 76 | 12 |
| AD-1446291.1 | 60 | 9 | 69 | 5 | 89 | 16 |
| AD-1446292.1 | 23 | 2 | 32 | 5 | 38 | 6 |
| AD-1446293.1 | 20 | 6 | 32 | 4 | 44 | 4 |
| AD-1446294.1 | 11 | 2 | 21 | 2 | 33 | 4 |
| AD-1446295.1 | 23 | 5 | 34 | 6 | 53 | 6 |

*“message” for this example is an antisense transcript

TABLE 4A
C90RF72 INTRON-1A Antisense RNA target sequences having ≤50% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5523 | 5571 | CAGCTTCGGTCAGAGAAATGAGAGGGAAAGTAA | 21 |
| | | AAATGCGTCGAGCTCT | |
| 5283 | 5315 | CGCGACTCCTGAGTTCCAGAGCTTGCTACAGGC | 22 |
| 5260 | 5286 | AGGCTGCGGTTGTTTCCCTCCTTGTTT | 23 |
| 5201 | 5279 | GGTTGTTTCCCTCCTTGTTTTCTTCTGGTTAATCTT | 24 |
| | | TATCAGGTCTTTTCTTGTTCACCCTCAGCGAGTAC | |
| | | TGTGAGAG | |
| 5197 | 5220 | CTCAGCGAGTACTGTGAGAGCAAG | 25 |
| 5128 | 5160 | ACCTCCTAAACCCACACCTGCTCTTGCTAGACC | 26 |

TABLE 4B
C9ORF72 INTRON-1A Antisense RNA target sequences having ≤40% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5524 | 5571 | CAGCTTCGGTCAGAGAAATGAGAGGGAAAGTAA | 27 |
| | | AAATGCGTCGAGCTC | |
| 5292 | 5315 | CGCGACTCCTGAGTTCCAGAGCTT | 28 |
| 5283 | 5312 | GACTCCTGAGTTCCAGAGCTTGCTACAGGC | 29 |
| 5260 | 5285 | GGCTGCGGTTGTTTCCCTCCTTGTTT | 30 |
| 5201 | 5279 | GGTTGTTTCCCTCCTTGTTTTCTTCTGGTTAATCTT | 31 |
| | | TATCAGGTCTTTTCTTGTTCACCCTCAGCGAGTAC | |
| | | TGTGAGAG | |

TABLE 4C
C9ORF72 INTRON-1A Antisense RNA target sequences having ≤30% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5526 | 5571 | CAGCTTCGGTCAGAGAAATGAGAGGGAAAGTAA | 32 |
| | | AAATGCGTCGAGC | |
| 5285 | 5311 | ACTCCTGAGTTCCAGAGCTTGCTACAG | 33 |
| 5260 | 5283 | CTGCGGTTGTTTCCCTCCTTGTTT | 34 |
| 5243 | 5279 | GGTTGTTTCCCTCCTTGTTTTCTTCTGGTTAATCT | 35 |
| | | TT | |
| 5212 | 5261 | TTTCTTCTGGTTAATCTTTATCAGGTCTTTTCTTG | 36 |
| | | TTCACCCTCAGCGAG | |

TABLE 4D
C9ORF72 INTRON-1A Antisense RNA target sequences having ≤25% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5529 | 5552 | GAGAGGGAAAGTAAAAATGCGTCG | 37 |
| 5243 | 5279 | GGTTGTTTCCCTCCTTGTTTTCTTCTGGTTAATCTT | 38 |
| | | T | |
| 5214 | 5261 | TTTCTTCTGGTTAATCTTTATCAGGTCTTTTCTTGT | 39 |
| | | TCACCCTCAGCG | |

TABLE 4E
C9ORF72 INTRON-1A Antisense RNA target sequences having ≤20% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5243 | 5275 | GTTTCCCTCCTTGTTTTCTTCTGGTTAATCTTT | 40 |
| 5215 | 5261 | TTTCTTCTGGTTAATCTTTATCAGGTCTTTTCTT | 41 |
| | | GTTCACCCTCAGC | |

TABLE 4F
C9ORF72 INTRON-1A Antisense RNA target sequences having ≤15% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5250 | 5275 | GTTTCCCTCCTTGTTTTCTTCTGGTT | 42 |
| 5243 | 5269 | CTCCTTGTTTTCTTCTGGTTAATCTTT | 43 |
| 5220 | 5261 | TTTCTTCTGGTTAATCTTTATCAGGTCTTTTCTT | 44 |
| | | GTTCACCC | |

TABLE 4G
C9ORF72 INTRON-1A Antisense RNA target sequences having ≤10% antisense
transcript remaining for dosing at 0.1 nM as measured in Table 4.

| | | | SEQ |
|---|---|---|---|
| Target | Target | RNA Target Sequence | ID |
| Start | End | (Reverse Complement of NG_031977.2) | NO.: |
| 5243 | 5268 | TCCTTGTTTTCTTCTGGTTAATCTTT | 45 |
| 5228 | 5261 | TTTCTTCTGGTTAATCTTTATCAGGTCTTTTCTT | 46 |
| 5220 | 5248 | ATCTTTATCAGGTCTTTTCTTGTTCACCC | 47 |

TABLE 5
Unmodified Sense and Antisense Strand Sequences of dsRNA Agents Targeting the Sense Strand of Either Exon 1A or Downstream of
the Intronic Repeat Between Exons 1A and 1B¹.

| | | SEQ | | | | SEQ | | |
|---|---|---|---|---|---|---|---|---|
| Duplex | Sense Sequence | ID | Range | Range | | ID | Range | Range |
| Name | 5′ to 3′ | NO: | (NM_001256054.2) | (NG_031977.2) | Antisense Sequence 5′ to 3′ | NO: | (NM_001256054.2) | (NG_031977.2) |
| AD- | GUAACCUACGGUGUCC | 559 | 3 to 23 | 5003-5023 | UAGCGGGACACCGUAG | 707 | 1 to 23 | 5001-5023 |
| 1446073.1 | CGCUA | | | | GUUACGU | | | |
| AD- | GUCCCGCUAGGAAAGA | 560 | 15-35 | 5015-5035 | UCCUCUCUUUCCUAGCG | 708 | 13-35 | 5013-5035 |
| 1446074.1 | GAGGA | | | | GGACAC | | | |
| AD- | CCCGCUAGGAAAGAGA | 561 | 17-37 | 5017-5037 | UCACCUCUCUUUCCUAG | 709 | 15-37 | 5015-5037 |
| 1446075.1 | GGUGA | | | | CGGGAC | | | |
| AD- | CGCUAGGAAAGAGAG | 562 | 19-39 | 5019-5039 | UCGCACCUCUCUUUCCU | 710 | 17-39 | 5017-5039 |
| 1446076.1 | GUGCGA | | | | AGCGGG | | | |
| AD- | GCUAGGAAAGAGAGG | 563 | 20-40 | 5020-5040 | UACGCACCUCUCUUUCC | 711 | 18-40 | 5018-5040 |
| 1446077.1 | UGCGUA | | | | UAGCGG | | | |
| AD- | UAGGAAAGAGAGGUG | 564 | 22-42 | 5022-5042 | UUGACGCACCUCUCUUU | 712 | 20-42 | 5020-5042 |
| 1446078.1 | CGUCAA | | | | CCUAGC | | | |
| AD- | AGGUGCGUCAAACAGC | 565 | 32-52 | 5032-5052 | UUGUCGCUGUUUGACG | 713 | 30-52 | 5030-5052 |
| 1446079.1 | GACAA | | | | CACCUCU | | | |
| AD- | GGUGCGUCAAACAGCG | 566 | 33-53 | 5033-5053 | UUUGUCGCUGUUUGAC | 714 | 31-53 | 5031-5053 |
| 1446080.1 | ACAAA | | | | GCACCUC | | | |
| AD- | GUGCGUCAAACAGCGA | 567 | 34-54 | 5034-5054 | UCUUGUCGCUGUUUGA | 715 | 32-54 | 5032-5054 |
| 1446081.1 | CAAGA | | | | CGCACCU | | | |
| AD- | UGCGUCAAACAGCGAC | 568 | 35-55 | 5035-5055 | UACUUGTCGCUGUUUG | 716 | 33-55 | 5033-5055 |
| 1285246.2 | AAGUA | | | | ACGCACC | | | |
| AD- | UGCGUCAAACAGCGAC | 568 | 35-55 | 5035-5055 | UACUUGTCGCUGUUUG | 716 | 33-55 | 5033-5055 |
| 1285246.1 | AAGUA | | | | ACGCACC | | | |
| AD- | GCGUCAAACAGCGACA | 569 | 36-56 | 5036-5056 | UAACUUGUCGCUGUUU | 717 | 34-56 | 5034-5056 |
| 1446082.1 | AGUUA | | | | GACGCAC | | | |
| AD- | CGUCAAACAGCGACAA | 570 | 37-57 | 5037-5057 | UGAACUTGUCGCUGUU | 718 | 35-57 | 5035-5057 |
| 1285245.2 | GUUCA | | | | UGACGCA | | | |
| AD- | CGUCAAACAGCGACAA | 570 | 37-57 | 5037-5057 | UGAACUTGUCGCUGUU | 718 | 35-57 | 5035-5057 |
| 1285245.1 | GUUCA | | | | UGACGCA | | | |
| AD- | GUCAAACAGCGACAAG | 571 | 38-58 | 5038-5058 | UGGAACTUGUCGCUGU | 719 | 36-58 | 5036-5058 |
| 1446083.1 | UUCCA | | | | UUGACGC | | | |
| AD- | UCAAACAGCGACAAGU | 572 | 39-59 | 5039-5059 | UCGGAACUUGUCGCUG | 720 | 37-59 | 5037-5059 |
| 1446084.1 | UCCGA | | | | UUUGACG | | | |
| AD- | CCGCCCACGUAAAAGA | 573 | 56-76 | 5056-5076 | UGUCAUCUUUUACGUG | 721 | 54-76 | 5054-5076 |
| 1446085.1 | UGACA | | | | GGCGGAA | | | |
| AD- | CCACGUAAAAGAUGAC | 574 | 60-80 | 5060-5080 | UAAGCGUCAUCUUUUA | 722 | 58-80 | 5058-5080 |
| 1446086.1 | GCUUA | | | | CGUGGGC | | | |
| AD- | CCACGUAAAAGAUGAC | 574 | 60-80 | 5060-5080 | UAAGCGTCAUCUUUUAC | 723 | 58-80 | 5058-5080 |
| 1285247.1 | GCUUA | | | | GUGGGC | | | |
| AD- | CACGUAAAAGAUGACG | 575 | 61-81 | 5061-5081 | UCAAGCGUCAUCUUUU | 724 | 59-81 | 5059-5081 |
| 1446087.1 | CUUGA | | | | ACGUGGG | | | |
| AD- | ACGUAAAAGAUGACGC | 576 | 62-82 | 5062-5082 | UCCAAGCGUCAUCUUU | 725 | 60-82 | 5060-5082 |
| 1446088.1 | UUGGA | | | | UACGUGG | | | |
| AD- | CGUAAAAGAUGACGCU | 577 | 63-83 | 5063-5083 | UACCAAGCGUCAUCUU | 726 | 61-83 | 5061-5083 |
| 1446089.1 | UGGUA | | | | UUACGUG | | | |
| AD- | GUAAAAGAUGACGCU | 578 | 64-84 | 5064-5084 | UCACCAAGCGUCAUCUU | 727 | 62-84 | 5062-5084 |
| 1446090.1 | UGGUGA | | | | UUACGU | | | |
| AD- | UAAAAGAUGACGCUU | 579 | 65-85 | 5065-5085 | UACACCAAGCGUCAUCU | 728 | 63-85 | 5063-5085 |
| 1446091.1 | GGUGUA | | | | UUUACG | | | |
| AD- | AAAAGAUGACGCUUG | 580 | 66-86 | 5066-5086 | UCACACCAAGCGUCAUC | 729 | 64-86 | 5064-5086 |
| 1446092.1 | GUGUGA | | | | UUUUAC | | | |
| AD- | AAAGAUGACGCUUGG | 581 | 67-87 | 5067-5087 | UACACACCAAGCGUCAU | 730 | 65-87 | 5065-5087 |
| 1446093.1 | UGUGUA | | | | CUUUUA | | | |
| AD- | AGAUGACGCUUGGUG | 582 | 69-89 | 5069-5089 | UUGACACACCAAGCGUC | 731 | 67-89 | 5067-5089 |
| 1446094.1 | UGUCAA | | | | AUCUUU | | | |
| AD- | AUGACGCUUGGUGUG | 583 | 71-91 | 5071-5091 | UGCUGACACACCAAGCG | 732 | 69-91 | 5069-5091 |
| 1446095.1 | UCAGCA | | | | UCAUCU | | | |
| AD- | GACGCUUGGUGUGUCA | 584 | 73-93 | 5073-5093 | UCGGCUGACACACCAAG | 733 | 71-93 | 5071-5093 |
| 1446096.1 | GCCGA | | | | CGUCAU | | | |
| AD- | GCUGCCCGGUUGCUUC | 585 | 98-118 | 5098-5118 | UAAGAGAAGCAACCGG | 734 | 96-118 | 5096-5118 |
| 1446097.1 | UCUUA | | | | GCAGCAG | | | |
| AD- | GUCUAGCAAGAGCAGG | 586 | 129-149 | 5129-5149 | UCACACCUGCUCUUGCU | 735 | 127-149 | 5127-5149 |
| 1446098.1 | UGUGA | | | | AGACCC | | | |
| AD- | GCAGGUGUGGGUUUA | 587 | 140-160 | 5140-5160 | UCCUCCUAAACCCACAC | 736 | 138-160 | 5138-5160 |
| 1446099.1 | GGAGGA | | | | CUGCUC | | | |
| AD- | CAGGUGUGGGUUUAG | 588 | 141-161 | 5141-5161 | UACCUCCUAAACCCACA | 737 | 139-161 | 5139-5161 |
| 1446100.1 | GAGGUA | | | | CCUGCU | | | |
| AD- | AGGUGUGGGUUUAGG | 589 | 142-162 | 5142-5162 | UCACCUCCUAAACCCAC | 738 | 140-162 | 5140-5162 |
| 1446101.1 | AGGUGA | | | | ACCUGC | | | |
| AD- | GUGUGGGUUUAGGAG | 590 | 144-164 | 5144-5164 | UCACACCUCCUAAACCC | 739 | 142-164 | 5142-5164 |
| 1446102.1 | GUGUGA | | | | ACACCU | | | |
| AD- | UGCUCUCACAGUACUC | 591 | | 5199-5219 | UCAGCGAGUACUGUGA | 1292 | | 5197-5219 |
| 1285244.1 | GCUGA | | | | GAGCAAG | | | |
| AD- | UCUCACAGUACUCGCU | 592 | | 5202-5222 | UCCUCAGCGAGUACUG | 740 | | 5200-5222 |
| 1446103.1 | GAGGA | | | | UGAGAGC | | | |
| AD- | UCACAGUACUCGCUGA | 593 | | 5204-5224 | UACCCUCAGCGAGUACU | 741 | | 5202-5224 |
| 1446104.1 | GGGUA | | | | GUGAGA | | | |
| AD- | GCUGAGGGUGAACAA | 594 | | 5215-5235 | UUUUUCUUGUUCACCC | 742 | | 5213-5235 |
| 1285235.1 | GAAAAA | | | | UCAGCGA | | | |
| AD- | AACAAGAAAAGACCUG | 595 | | 5225-5245 | UUUAUCAGGUCUUUUC | 1422 | | 5223-5245 |
| 1285238.1 | AUAAA | | | | UUGUUCA | | | |
| AD- | AAGAAAAGACCUGAU | 596 | | 5228-5248 | UUCUUUAUCAGGUCUU | 743 | | 5226-5248 |
| 1285243.1 | AAAGAA | | | | UUCUUGU | | | |
| AD- | AGAAAAGACCUGAUA | 597 | | 5229-5249 | UAUCUUUAUCAGGUCU | 744 | | 5227-5249 |
| 1285234.1 | AAGAUA | | | | UUUCUUG | | | |
| AD- | GAAAAGACCUGAUAA | 598 | | 5230-5250 | UAAUCUUUAUCAGGUC | 1420 | | 5228-5250 |
| 1285239.1 | AGAUUA | | | | UUUUCUU | | | |
| AD- | AAAAGACCUGAUAAA | 599 | | 5231-5251 | UUAAUCUUUAUCAGGU | 1419 | | 5229-5251 |
| 1285232.1 | GAUUAA | | | | CUUUUCU | | | |
| AD- | AAAGACCUGAUAAAG | 600 | | 5232-5252 | UUUAAUCUUUAUCAGG | 745 | | 5230-5252 |
| 1285231.1 | AUUAAA | | | | UCUUUUC | | | |
| AD- | AAGACCUGAUAAAGA | 601 | | 5233-5253 | UGUUAAUCUUUAUCAG | 746 | | 5231-5253 |
| 1285240.1 | UUAACA | | | | GUCUUUU | | | |
| AD- | GACCUGAUAAAGAUU | 602 | | 5235-5255 | UUGGUUAAUCUUUAUC | 747 | | 5233-5255 |
| 1285241.1 | AACCAA | | | | AGGUCUU | | | |
| AD- | ACCUGAUAAAGAUUA | 603 | | 5236-5256 | UCUGGUUAAUCUUUAU | 1418 | | 5234-5256 |
| 1285242.1 | ACCAGA | | | | CAGGUCU | | | |
| AD- | AAAGAUUAACCAGAA | 604 | | 5243-5263 | UUUUUCUUCUGGUUAA | 748 | | 5241-5263 |
| 1285233.1 | GAAAAA | | | | UCUUUAU | | | |
| AD- | AUUAACCAGAAGAAA | 605 | | 5247-5267 | UCUUGUUUUCUUCUGG | 749 | | 5245-5267 |
| 1285237.1 | ACAAGA | | | | UUAAUCU | | | |
| AD- | AACCAGAAGAAAACAA | 606 | | 5250-5270 | UCUCCUUGUUUUCUUC | 750 | | 5248-5270 |
| 1285236.1 | GGAGA | | | | UGGUUAA | | | |
| AD- | GGAGGGAAACAACCGC | 607 | | 5266-5286 | UGGCUGCGGUUGUUUC | 751 | | 5264-5286 |
| 1446105.1 | AGCCA | | | | CCUCCUU | | | |
| AD- | GAGGGAAACAACCGCA | 608 | | 5267-5287 | UAGGCUGCGGUUGUUU | 752 | | 5265-5287 |
| 1446106.1 | GCCUA | | | | CCCUCCU | | | |
| AD- | CAGCCUGUAGCAAGCU | 609 | | 5281-5301 | UCCAGAGCUUGCUACA | 1415 | | 5279-5301 |
| 1446107.1 | CUGGA | | | | GGCUGCG | | | |
| AD- | CUCUGGAACUCAGGAG | 610 | | 5295-5315 | UGCGACTCCUGAGUUCC | 753 | | 5293-5315 |
| 1446108.1 | UCGCA | | | | AGAGCU | | | |
| AD- | UCUCCUCAGAGCUCGA | 611 | | 5516-5536 | UUGCGUCGAGCUCUGA | 754 | | 5514-5536 |
| 1446109.1 | CGCAA | | | | GGAGAGC | | | |
| AD- | UCCUCAGAGCUCGACG | 612 | | 5518-5538 | UAAUGCGUCGAGCUCU | 755 | | 5516-5538 |
| 1446110.1 | CAUUA | | | | GAGGAGA | | | |
| AD- | ACUUUCCCUCUCAUUU | 613 | | 5541-5561 | UAGAGAAAUGAGAGGG | 756 | | 5539-5561 |
| 1446111.1 | CUCUA | | | | AAAGUAA | | | |
| AD- | CUUUCCCUCUCAUUUC | 614 | | 5542-5562 | UCAGAGAAAUGAGAGG | 757 | | 5540-5562 |
| 1446112.1 | UCUGA | | | | GAAAGUA | | | |
| AD- | UUUCCCUCUCAUUUCU | 615 | | 5543-5563 | UUCAGAGAAAUGAGAG | 758 | | 5541-5563 |
| 1446113.1 | CUGAA | | | | GGAAAGU | | | |
| AD- | UCCCUCUCAUUUCUCU | 616 | | 5545-5565 | UGGUCAGAGAAAUGAG | 759 | | 5543-5565 |
| 1446114.1 | GACCA | | | | AGGGAAA | | | |
| AD- | CCCUCUCAUUUCUCUG | 617 | | 5546-5566 | UCGGUCAGAGAAAUGA | 1414 | | 5544-5566 |
| 1446115.1 | ACCGA | | | | GAGGGAA | | | |
| AD- | CCUCUCAUUUCUCUGA | 618 | | 5547-5567 | UUCGGUCAGAGAAAUG | 760 | | 5545-5567 |
| 1446116.1 | CCGAA | | | | AGAGGGA | | | |
| AD- | UCUCAUUUCUCUGACC | 619 | | 5549-5569 | UCUUCGGUCAGAGAAA | 761 | | 5547-5569 |
| 1446117.1 | GAAGA | | | | UGAGAGG | | | |
| AD- | CUCAUUUCUCUGACCG | 620 | | 5550-5570 | UGCUUCGGUCAGAGAA | 762 | | 5548-5570 |
| 1446118.1 | AAGCA | | | | AUGAGAG | | | |
| AD- | UCAUUUCUCUGACCGA | 621 | | 5551-5571 | UAGCUUCGGUCAGAGA | 763 | | 5549-5571 |
| 1446119.1 | AGCUA | | | | AAUGAGA | | | |
| AD- | CUCUGACCGAAGCUGG | 622 | | 5557-5577 | UACACCCAGCUUCGGUC | 764 | | 5555-5577 |
| 1446120.1 | GUGUA | | | | AGAGAA | | | |
| AD- | GGUGUCGGGCUUUCGC | 623 | | 5572-5592 | UAGAGGCGAAAGCCCG | 765 | | 5570-5592 |
| 1446121.1 | CUCUA | | | | ACACCCA | | | |
| AD- | UCGGGCUUUCGCCUCU | 624 | | 5576-5596 | UCGCUAGAGGCGAAAG | 766 | | 5574-5596 |
| 1446122.1 | AGCGA | | | | CCCGACA | | | |
| AD- | CUUUCGCCUCUAGCGA | 625 | | 5581-5601 | UCCAGUCGCUAGAGGC | 767 | | 5579-5601 |
| 1446123.1 | CUGGA | | | | GAAAGCC | | | |
| AD- | UUCGCCUCUAGCGACU | 626 | | 5583-5603 | UCACCAGUCGCUAGAG | 768 | | 5581-5603 |
| 1446124.1 | GGUGA | | | | GCGAAAG | | | |
| AD- | UCGCCUCUAGCGACUG | 627 | | 5584-5604 | UCCACCAGUCGCUAGAG | 1413 | | 5582-5604 |
| 1446125.1 | GUGGA | | | | GCGAAA | | | |
| AD- | CGCCUCUAGCGACUGG | 628 | | 5585-5605 | UUCCACCAGUCGCUAGA | 769 | | 5583-5605 |
| 1446126.1 | UGGAA | | | | GGCGAA | | | |
| AD- | GCCUCUAGCGACUGGU | 629 | | 5586-5606 | UUUCCACCAGUCGCUAG | 770 | | 5584-5606 |
| 1446127.1 | GGAAA | | | | AGGCGA | | | |
| AD- | CUCUAGCGACUGGUGG | 630 | | 5588-5608 | UAAUUCCACCAGUCGCU | 771 | | 5586-5608 |
| 1446128.1 | AAUUA | | | | AGAGGC | | | |
| AD- | UCUAGCGACUGGUGGA | 631 | | 5589-5609 | UCAAUUCCACCAGUCGC | 772 | | 5587-5609 |
| 1446129.1 | AUUGA | | | | UAGAGG | | | |
| AD- | GACUGGUGGAAUUGCC | 632 | | 5595-5615 | UUGCAGGCAAUUCCACC | 773 | | 5593-5615 |
| 1446130.1 | UGCAA | | | | AGUCGC | | | |
| AD- | ACUGGUGGAAUUGCCU | 633 | | 5596-5616 | UAUGCAGGCAAUUCCA | 774 | | 5594-5616 |
| 1446131.1 | GCAUA | | | | CCAGUCG | | | |
| AD- | UGGUGGAAUUGCCUGC | 634 | | 5598-5618 | UGGAUGCAGGCAAUUC | 775 | | 5596-5618 |
| 1446132.1 | AUCCA | | | | CACCAGU | | | |
| AD- | UCUGGCCUCUUCCUUG | 635 | | 5678-5698 | UAAAGCAAGGAAGAGG | 776 | | 5676-5698 |
| 1446134.1 | CUUUA | | | | CCAGAUC | | | |
| AD- | CUGGCCUCUUCCUUGC | 636 | | 5679-5699 | UGAAAGCAAGGAAGAG | 777 | | 5677-5699 |
| 1446135.1 | UUUCA | | | | GCCAGAU | | | |
| AD- | UGGCCUCUUCCUUGCU | 637 | | 5680-5700 | UGGAAAGCAAGGAAGA | 778 | | 5678-5700 |
| 1446136.1 | UUCCA | | | | GGCCAGA | | | |
| AD- | GGCCUCUUCCUUGCUU | 638 | | 5681-5701 | UGGGAAAGCAAGGAAG | 779 | | 5679-5701 |
| 1446137.1 | UCCCA | | | | AGGCCAG | | | |
| AD- | GCCUCUUCCUUGCUUU | 639 | | 5682-5702 | UCGGGAAAGCAAGGAA | 780 | | 5680-5702 |
| 1446138.1 | CCCGA | | | | GAGGCCA | | | |
| AD- | UUCCUUGCUUUCCCGC | 640 | | 5687-5707 | UGAGGGCGGGAAAGCA | 781 | | 5685-5707 |
| 1446139.1 | CCUCA | | | | AGGAAGA | | | |
| AD- | UCCUUGCUUUCCCGCC | 641 | | 5688-5708 | UUGAGGGCGGGAAAGC | 782 | | 5686-5708 |
| 1446140.1 | CUCAA | | | | AAGGAAG | | | |
| AD- | CCUUGCUUUCCCGCCC | 642 | | 5689-5709 | UCUGAGGGCGGGAAAG | 783 | | 5687-5709 |
| 1446141.1 | UCAGA | | | | CAAGGAA | | | |
| AD- | CUUGCUUUCCCGCCCU | 643 | | 5690-5710 | UACUGAGGGCGGGAAA | 784 | | 5688-5710 |
| 1446142.1 | CAGUA | | | | GCAAGGA | | | |
| AD- | AGUACCCGAGCUGUCU | 644 | | 5707-5727 | UAAGGAGACAGCUCGG | 785 | | 5705-5727 |
| 1446143.1 | CCUUA | | | | GUACUGA | | | |
| AD- | UACCCGAGCUGUCUCC | 645 | | 5709-5729 | UGGAAGGAGACAGCUC | 786 | | 5707-5729 |
| 1446144.1 | UUCCA | | | | GGGUACU | | | |
| AD- | GAGGAGAUCAUGCGG | 646 | | 5885-5905 | UUCAUCCCGCAUGAUCU | 787 | | 5883-5905 |
| 1446145.1 | GAUGAA | | | | CCUCGC | | | |
| AD- | AGACGCCUGCACAAUU | 647 | | 5918-5938 | UCUGAAAUUGUGCAGG | 788 | | 5916-5938 |
| 1446146.1 | UCAGA | | | | CGUCUCC | | | |
| AD- | GACGCCUGCACAAUUU | 648 | | 5919-5939 | UGCUGAAAUUGUGCAG | 789 | | 5917-5939 |
| 1446147.1 | CAGCA | | | | GCGUCUC | | | |
| AD- | CGCCUGCACAAUUUCA | 649 | | 5921-5941 | UGGGCUGAAAUUGUGC | 790 | | 5919-5941 |
| 1446148.1 | GCCCA | | | | AGGCGUC | | | |
| AD- | CCUGCACAAUUUCAGC | 650 | | 5923-5943 | UUUGGGCUGAAAUUGU | 791 | | 5921-5943 |
| 1446149.1 | CCAAA | | | | GCAGGCG | | | |
| AD- | CUGCACAAUUUCAGCC | 651 | | 5924-5944 | UCUUGGGCUGAAAUUG | 792 | | 5922-5944 |
| 1446150.1 | CAAGA | | | | UGCAGGC | | | |
| AD- | UGCACAAUUUCAGCCC | 652 | | 5925-5945 | UGCUUGGGCUGAAAUU | 793 | | 5923-5945 |
| 1446151.1 | AAGCA | | | | GUGCAGG | | | |
| AD- | CAAUUUCAGCCCAAGC | 653 | | 5929-5949 | UAGAAGCUUGGGCUGA | 794 | | 5927-5949 |
| 1446152.1 | UUCUA | | | | AAUUGUG | | | |
| AD- | AAUUUCAGCCCAAGCU | 654 | | 5930-5950 | UUAGAAGCUUGGGCUG | 795 | | 5928-5950 |
| 1446153.1 | UCUAA | | | | AAAUUGU | | | |
| AD- | CAGCCCAAGCUUCUAG | 655 | | 5935-5955 | UCUCUCTAGAAGCUUGG | 796 | | 5933-5955 |
| 1446154.1 | AGAGA | | | | GCUGAA | | | |
| AD- | AGCCCAAGCUUCUAGA | 656 | | 5936-5956 | UACUCUCUAGAAGCUU | 797 | | 5934-5956 |
| 1446155.1 | GAGUA | | | | GGGCUGA | | | |
| AD- | GCCCAAGCUUCUAGAG | 657 | | 5937-5957 | UCACUCTCUAGAAGCUU | 798 | | 5935-5957 |
| 1446156.1 | AGUGA | | | | GGGCUG | | | |
| AD- | CCCAAGCUUCUAGAGA | 658 | | 5938-5958 | UCCACUCUCUAGAAGCU | 799 | | 5936-5958 |
| 1446157.1 | GUGGA | | | | UGGGCU | | | |
| AD- | CCAAGCUUCUAGAGAG | 659 | | 5939-5959 | UACCACTCUCUAGAAGC | 800 | | 5937-5959 |
| 1446158.1 | UGGUA | | | | UUGGGC | | | |
| AD- | CAAGCUUCUAGAGAGU | 660 | | 5940-5960 | UCACCACUCUCUAGAAG | 801 | | 5938-5960 |
| 1446159.1 | GGUGA | | | | CUUGGG | | | |
| AD- | AAGCUUCUAGAGAGU | 661 | | 5941-5961 | UUCACCACUCUCUAGAA | 802 | | 5939-5961 |
| 1446160.1 | GGUGAA | | | | GCUUGG | | | |
| AD- | AGCUUCUAGAGAGUG | 662 | | 5942-5962 | UAUCACCACUCUCUAGA | 803 | | 5940-5962 |
| 1446161.1 | GUGAUA | | | | AGCUUG | | | |
| AD- | GCUUCUAGAGAGUGG | 663 | | 5943-5963 | UCAUCACCACUCUCUAG | 804 | | 5941-5963 |
| 1446162.1 | UGAUGA | | | | AAGCUU | | | |
| AD- | UUCUAGAGAGUGGUG | 664 | | 5945-5965 | UGUCAUCACCACUCUCU | 805 | | 5943-5965 |
| 1446163.1 | AUGACA | | | | AGAAGC | | | |
| AD- | UAGAGAGUGGUGAUG | 665 | | 5948-5968 | UCAAGUCAUCACCACUC | 806 | | 5946-5968 |
| 1446164.1 | ACUUGA | | | | UCUAGA | | | |
| AD- | AGAGAGUGGUGAUGA | 666 | | 5949-5969 | UGCAAGTCAUCACCACU | 807 | | 5947-5969 |
| 1446165.1 | CUUGCA | | | | CUCUAG | | | |
| AD- | GAGAGUGGUGAUGAC | 667 | | 5950-5970 | UUGCAAGUCAUCACCAC | 808 | | 5948-5970 |
| 1446166.1 | UUGCAA | | | | UCUCUA | | | |
| AD- | AGUGGUGAUGACUUG | 668 | | 5953-5973 | UAUAUGCAAGUCAUCA | 809 | | 5951-5973 |
| 1446167.1 | CAUAUA | | | | CCACUCU | | | |
| AD- | GGUGAUGACUUGCAU | 669 | | 5956-5976 | UCUCAUAUGCAAGUCA | 810 | | 5954-5976 |
| 1446168.1 | AUGAGA | | | | UCACCAC | | | |
| AD- | GUGAUGACUUGCAUA | 670 | | 5957-5977 | UCCUCAUAUGCAAGUC | 811 | | 5955-5977 |
| 1446169.1 | UGAGGA | | | | AUCACCA | | | |
| AD- | UGAUGACUUGCAUAU | 671 | | 5958-5978 | UCCCUCAUAUGCAAGUC | 812 | | 5956-5978 |
| 1446170.1 | GAGGGA | | | | AUCACC | | | |
| AD- | AGGGCAGCAAUGCAAG | 672 | | 5974-5994 | UCCGACUUGCAUUGCU | 813 | | 5972-5994 |
| 1446171.1 | UCGGA | | | | GCCCUCA | | | |
| AD- | GGGCAGCAAUGCAAGU | 673 | | 5975-5995 | UACCGACUUGCAUUGC | 814 | | 5973-5995 |
| 1446172.1 | CGGUA | | | | UGCCCUC | | | |
| AD- | GGCAGCAAUGCAAGUC | 674 | | 5976-5996 | UCACCGACUUGCAUUGC | 815 | | 5974-5996 |
| 1446173.1 | GGUGA | | | | UGCCCU | | | |
| AD- | GCAGCAAUGCAAGUCG | 675 | | 5977-5997 | UACACCGACUUGCAUU | 816 | | 5975-5997 |
| 1446174.1 | GUGUA | | | | GCUGCCC | | | |
| AD- | CAGCAAUGCAAGUCGG | 676 | | 5978-5998 | UCACACCGACUUGCAUU | 817 | | 5976-5998 |
| 1446175.1 | UGUGA | | | | GCUGCC | | | |
| AD- | CAAUGCAAGUCGGUGU | 677 | | 5981-6001 | UGAGCACACCGACUUGC | 818 | | 5979-6001 |
| 1446176.1 | GCUCA | | | | AUUGCU | | | |
| AD- | CUGUGGGACAUGACCU | 678 | | 6007-6027 | UAACCAGGUCAUGUCCC | 819 | | 6005-6027 |
| 1446177.1 | GGUUA | | | | ACAGAA | | | |
| AD- | UGUGGGACAUGACCUG | 679 | | 6008-6028 | UCAACCAGGUCAUGUCC | 820 | | 6006-6028 |
| 1446178.1 | GUUGA | | | | CACAGA | | | |
| AD- | GUGGGACAUGACCUGG | 680 | | 6009-6029 | UGCAACCAGGUCAUGU | 821 | | 6007-6029 |
| 1446179.1 | UUGCA | | | | CCCACAG | | | |
| AD- | UGGGACAUGACCUGGU | 681 | | 6010-6030 | UAGCAACCAGGUCAUG | 822 | | 6008-6030 |
| 1446180.1 | UGCUA | | | | UCCCACA | | | |
| AD- | GACAUGACCUGGUUGC | 682 | | 6013-6033 | UUGAAGCAACCAGGUC | 823 | | 6011-6033 |
| 1446181.1 | UUCAA | | | | AUGUCCC | | | |
| AD- | ACAUGACCUGGUUGCU | 683 | | 6014-6034 | UGUGAAGCAACCAGGU | 824 | | 6012-6034 |
| 1446182.1 | UCACA | | | | CAUGUCC | | | |
| AD- | UGACCUGGUUGCUUCA | 684 | | 6017-6037 | UGCUGUGAAGCAACCA | 825 | | 6015-6037 |
| 1446183.1 | CAGCA | | | | GGUCAUG | | | |
| AD- | GACCUGGUUGCUUCAC | 685 | | 6018-6038 | UAGCUGTGAAGCAACCA | 826 | | 6016-6038 |
| 1446184.1 | AGCUA | | | | GGUCAU | | | |
| AD- | ACCUGGUUGCUUCACA | 686 | | 6019-6039 | UGAGCUGUGAAGCAAC | 827 | | 6017-6039 |
| 1446185.1 | GCUCA | | | | CAGGUCA | | | |
| AD- | CCUGGUUGCUUCACAG | 687 | | 6020-6040 | UGGAGCTGUGAAGCAA | 828 | | 6018-6040 |
| 1446186.1 | CUCCA | | | | CCAGGUC | | | |
| AD- | CUGGUUGCUUCACAGC | 688 | | 6021-6041 | UCGGAGCUGUGAAGCA | 829 | | 6019-6041 |
| 1446187.1 | UCCGA | | | | ACCAGGU | | | |
| AD- | UGGUUGCUUCACAGCU | 689 | | 6022-6042 | UUCGGAGCUGUGAAGC | 830 | | 6020-6042 |
| 1446188.1 | CCGAA | | | | AACCAGG | | | |
| AD- | GGUUGCUUCACAGCUC | 690 | | 6023-6043 | UCUCGGAGCUGUGAAG | 831 | | 6021-6043 |
| 1446189.1 | CGAGA | | | | CAACCAG | | | |
| AD- | GUUGCUUCACAGCUCC | 691 | | 6024-6044 | UUCUCGGAGCUGUGAA | 832 | | 6022-6044 |
| 1446190.1 | GAGAA | | | | GCAACCA | | | |
| AD- | UUGCUUCACAGCUCCG | 692 | | 6025-6045 | UAUCUCGGAGCUGUGA | 833 | | 6023-6045 |
| 1446191.1 | AGAUA | | | | AGCAACC | | | |
| AD- | CAGCUCCGAGAUGACA | 693 | | 6033-6053 | UUCUGUGUCAUCUCGG | 834 | | 6031-6053 |
| 1446192.1 | CAGAA | | | | AGCUGUG | | | |
| AD- | GCUCCGAGAUGACACA | 694 | | 6035-6055 | UAGUCUGUGUCAUCUC | 835 | | 6033-6055 |
| 1446193.1 | GACUA | | | | GGAGCUG | | | |
| AD- | CUCCGAGAUGACACAG | 695 | | 6036-6056 | UAAGUCTGUGUCAUCUC | 836 | | 6034-6056 |
| 1446194.1 | ACUUA | | | | GGAGCU | | | |
| AD- | UCCGAGAUGACACAGA | 696 | | 6037-6057 | UCAAGUCUGUGUCAUC | 837 | | 6035-6057 |
| 1446195.1 | CUUGA | | | | UCGGAGC | | | |
| AD- | CCGAGAUGACACAGAC | 697 | | 6038-6058 | UGCAAGTCUGUGUCAUC | 838 | | 6036-6058 |
| 1446196.1 | UUGCA | | | | UCGGAG | | | |
| AD- | CGAGAUGACACAGACU | 698 | | 6039-6059 | UAGCAAGUCUGUGUCA | 839 | | 6037-6059 |
| 1446197.1 | UGCUA | | | | UCUCGGA | | | |
| AD- | GAGAUGACACAGACUU | 699 | | 6040-6060 | UAAGCAAGUCUGUGUC | 840 | | 6038-6060 |
| 1446198.1 | GCUUA | | | | AUCUCGG | | | |
| AD- | GAUGACACAGACUUGC | 700 | | 6042-6062 | UUUAAGCAAGUCUGUG | 841 | | 6040-6062 |
| 1446199.1 | UUAAA | | | | UCAUCUC | | | |
| AD- | AUGACACAGACUUGCU | 701 | | 6043-6063 | UUUUAAGCAAGUCUGU | 842 | | 6041-6063 |
| 1446200.1 | UAAAA | | | | GUCAUCU | | | |
| AD- | UGACACAGACUUGCUU | 702 | | 6044-6064 | UCUUUAAGCAAGUCUG | 843 | | 6042-6064 |
| 1446201.1 | AAAGA | | | | UGUCAUC | | | |
| AD- | GACACAGACUUGCUUA | 703 | | 6045-6065 | UCCUUUAAGCAAGUCU | 844 | | 6043-6065 |
| 1446202.1 | AAGGA | | | | GUGUCAU | | | |
| AD- | ACACAGACUUGCUUAA | 704 | | 6046-6066 | UUCCUUUAAGCAAGUC | 845 | | 6044-6066 |
| 1446203.1 | AGGAA | | | | UGUGUCA | | | |
| AD- | CAGACUUGCUUAAAGG | 705 | | 6049-6069 | UACUUCCUUUAAGCAA | 846 | | 6047-6069 |
| 1446204.1 | AAGUA | | | | GUCUGUG | | | |
| AD- | AGACUUGCUUAAAGG | 706 | | 6050-6070 | UCACUUCCUUUAAGCA | 847 | | 6048-6070 |
| 1446205.1 | AAGUGA | | | | AGUCUGU | | | |

¹Exons 1a and 1b correspond to positions 5001-5158 and 5386-5436 of NG_031977.2.

TABLE 6
Modified Sense and Antisense Strand Sequences of dsRNA Agents Targeting the Sense Strand of Either Exon 1A or the 3′-side of the
Intronic Repeat Between Exons 1A and 1B

| | | SEQ | | SEQ | | SEQ | | SEQ |
|---|---|---|---|---|---|---|---|---|
| Duplex | | ID | | ID | mRNA target sequence | ID | mRNA target sequence | ID |
| Name | Sense Seuence 5′ to 3′ | NO: | Antisense Sequence 5′ to 3′ | NO: | (NM_001256054.2) | NO: | (NG_031977.2) | NO: |
| AD- | gsusaac(Chd)UfaCfGfGfuguc | 848 | VPusAfsgcgg(G2p)acaccgUfaGfg | 996 | ACGUAACCUACGGU | 1145 | ACGUAACCUACGGUGUC | 1145 |
| 1446073.1 | ccgcsusa | | uuacsgsu | | GUCCCGCUA | | CCGCUA | |
| AD- | gsusccc(Ghd)CfuAfGfGfaaag | 849 | VPusCfscucu(C2p)uuuccuAfgCfg | 997 | GUGUCCCGCUAGGA | 1146 | GUGUCCCGCUAGGAAAG | 1146 |
| 1446074.1 | agagsgsa | | ggacsasc | | AAGAGAGGU | | AGAGGU | |
| AD- | cscscgc(Uhd)AfgGfAfAfaga | 850 | VPusCfsaccu(C2p)ucuuucCfuAfg | 998 | GUCCCGCUAGGAAA | 1147 | GUCCCGCUAGGAAAGAG | 1147 |
| 1446075.1 | gaggusgsa | | cgggsasc | | GAGAGGUGC | | AGGUGC | |
| AD- | csgscua(Ghd)GfaAfAfGfaga | 851 | VPusCfsgcaCfcUfCfucuuUfcCfua | 999 | CCCGCUAGGAAAGA | 1148 | CCCGCUAGGAAAGAGAG | 1148 |
| 1446076.1 | ggugcsgsa | | gcgsgsg | | GAGGUGCGU | | GUGCGU | |
| AD- | gscsuag(Ghd)AfaAfGfAfgag | 852 | VPusAfscgca(C2p)cucucuUfuCfc | 1000 | CCGCUAGGAAAGAG | 1149 | CCGCUAGGAAAGAGAGG | 1149 |
| 1446077.1 | gugcgsusa | | uagcsgsg | | AGGUGCGUC | | UGCGUC | |
| AD- | usasgga(Ahd)AfgAfGfAfggu | 853 | VPusUfsgacg(C2p)accucuCfuUfu | 1001 | GCUAGGAAAGAGAG | 1150 | GCUAGGAAAGAGAGGUG | 1150 |
| 1446078.1 | gcgucsasa | | ccuasgsc | | GUGCGUCAA | | CGUCAA | |
| AD- | asgsgug(Chd)GfuCfAfAfaca | 854 | VPusUfsgucg(C2p)uguuugAfcGfc | 1002 | AGAGGUGCGUCAAA | 1151 | AGAGGUGCGUCAAACAG | 1151 |
| 1446079.1 | gcgacsasa | | accuscsu | | CAGCGACAA | | CGACAA | |
| AD- | gsgsugc(Ghd)UfcAfAfAfcag | 855 | VPusUfsuguc(G2p)cuguuuGfaCfg | 1003 | GAGGUGCGUCAAAC | 1152 | GAGGUGCGUCAAACAGC | 1152 |
| 1446080.1 | cgacasasa | | caccsusc | | AGCGACAAG | | GACAAG | |
| AD- | gsusgcg(Uhd)CfaAfAfCfagc | 856 | VPusCfsuugu(C2p)gcuguuUfgAfc | 1004 | AGGUGCGUCAAACA | 1153 | AGGUGCGUCAAACAGCG | 1153 |
| 1446081.1 | gacaasgsa | | gcacscsu | | GCGACAAGU | | ACAAGU | |
| AD- | usgscgu(Chd)AfaAfCfAfgcg | 857 | VPusAfscuug(Tgn)cgcuguUfuGfa | 1005 | GGUGCGUCAAACAG | 1154 | GGUGCGUCAAACAGCGA | 1154 |
| 1285246.2 | acaagsusa | | cgcascsc | | CGACAAGUU | | CAAGUU | |
| AD- | usgscgu(Chd)AfaAfCfAfgcg | 857 | VPusAfscuug(Tgn)cgcuguUfuGfa | 1005 | GGUGCGUCAAACAG | 1154 | GGUGCGUCAAACAGCGA | 1154 |
| 1285246.1 | acaagsusa | | cgcascsc | | CGACAAGUU | | CAAGUU | |
| AD- | gscsguc(Ahd)AfaCfAfGfcgac | 858 | VPusAfsacuu(G2p)ucgcugUfuUfg | 1006 | GUGCGUCAAACAGC | 1155 | GUGCGUCAAACAGCGAC | 1155 |
| 1446082.1 | aagususa | | acgcsasc | | GACAAGUUC | | AAGUUC | |
| AD- | csgsuca(Ahd)AfcAfGfCfgaca | 859 | VPusGfsaacu(Tgn)gucgcuGfuUfu | 1007 | UGCGUCAAACAGCG | 1156 | UGCGUCAAACAGCGACA | 1156 |
| 1285245.2 | aguuscsa | | gacgscsa | | ACAAGUUCC | | AGUUCC | |
| AD- | csgsuca(Ahd)AfcAfGfCfgaca | 859 | VPusGfsaacu(Tgn)gucgcuGfuUfu | 1007 | UGCGUCAAACAGCG | 1156 | UGCGUCAAACAGCGACA | 1156 |
| 1285245.1 | aguuscsa | | gacgscsa | | ACAAGUUCC | | AGUUCC | |
| AD- | gsuscaa(Ahd)CfaGfCfGfacaa | 860 | VPusGfsgaac(Tgn)ugucgcUfgUfu | 1008 | GCGUCAAACAGCGA | 1157 | GCGUCAAACAGCGACAA | 1157 |
| 1446083.1 | guucscsa | | ugacsgsc | | CAAGUUCCG | | GUUCCG | |
| AD- | uscsaaa(Chd)AfgCfGfAfcaag | 861 | VPusCfsggaAfcUfUfgucgCfuGfu | 1009 | CGUCAAACAGCGAC | 1158 | CGUCAAACAGCGACAAG | 1158 |
| 1446084.1 | uuccsgsa | | uugascsg | | AAGUUCCGC | | UUCCGC | |
| AD- | cscsgcc(Chd)AfcGfUfAfaaag | 862 | VPusGfsucau(C2p)uuuuacGfuGfg | 1010 | UUCCGCCCACGUAA | 1159 | UUCCGCCCACGUAAAAG | 1159 |
| 1446085.1 | augascsa | | gcggsasa | | AAGAUGACG | | AUGACG | |
| AD- | cscsacg(Uhd)AfaAfAfGfauga | 863 | VPusAfsagcGfuCfAfucuuUfuAfc | 1011 | GCCCACGUAAAAGA | 1160 | GCCCACGUAAAAGAUGA | 1160 |
| 1446086.1 | cgcususa | | guggsgsc | | UGACGCUUG | | CGCUUG | |
| AD- | cscsacg(Uhd)AfaAfAfGfauga | 863 | VPusAfsagcg(Tgn)caucuuUfuAfc | 1012 | GCCCACGUAAAAGA | 1160 | GCCCACGUAAAAGAUGA | 1160 |
| 1285247.1 | cgcususa | | guggsgsc | | UGACGCUUG | | CGCUUG | |
| AD- | csascgu(Ahd)AfaAfGfAfuga | 864 | VPusCfsaagCfgUfCfaucuUfuUfac | 1013 | CCCACGUAAAAGAU | 1161 | CCCACGUAAAAGAUGAC | 1161 |
| 1446087.1 | cgcuusgsa | | gugsgsg | | GACGCUUGG | | GCUUGG | |
| AD- | ascsgua(Ahd)AfaGfAfUfgac | 865 | VPusCfscaag(C2p)gucaucUfuUfu | 1014 | CCACGUAAAAGAUG | 1162 | CCACGUAAAAGAUGACG | 1162 |
| 1446088.1 | gcuugsgsa | | acgusgsg | | ACGCUUGGU | | CUUGGU | |
| AD- | csgsuaa(Ahd)AfgAfUfGfacg | 866 | VPusAfsccaa(G2p)cgucauCfuUfu | 1015 | CACGUAAAAGAUGA | 1163 | CACGUAAAAGAUGACGC | 1163 |
| 1446089.1 | cuuggsusa | | uacgsusg | | CGCUUGGUG | | UUGGUG | |
| AD- | gsusaaa(Ahd)GfaUfGfAfcgc | 867 | VPusCfsaccAfaGfCfgucaUfcUfuu | 1016 | ACGUAAAAGAUGAC | 1164 | ACGUAAAAGAUGACGCU | 1164 |
| 1446090.1 | uuggusgsa | | uacsgsu | | GCUUGGUGU | | UGGUGU | |
| AD- | usasaaa(Ghd)AfuGfAfCfgcu | 868 | VPusAfscacCfaAfGfcgucAfuCfu | 1017 | CGUAAAAGAUGACG | 1165 | CGUAAAAGAUGACGCUU | 1165 |
| 1446091.1 | uggugsusa | | uuuascsg | | CUUGGUGUG | | GGUGUG | |
| AD- | asasaag(Ahd)UfgAfCfGfcuu | 869 | VPusCfsacaCfcAfAfgcguCfaUfcu | 1018 | GUAAAAGAUGACGC | 1166 | GUAAAAGAUGACGCUUG | 1166 |
| 1446092.1 | ggugusgsa | | uuusasc | | UUGGUGUGU | | GUGUGU | |
| AD- | asasaga(Uhd)GfaCfGfCfuugg | 870 | VPusAfscacAfcCfAfagcgUfcAfuc | 1019 | UAAAAGAUGACGCU | 1167 | UAAAAGAUGACGCUUGG | 1167 |
| 1446093.1 | ugugsusa | | uuususa | | UGGUGUGUC | | UGUGUC | |
| AD- | asgsaug(Ahd)CfgCfUfUfggu | 871 | VPusUfsgaca(C2p)accaagCfgUfc | 1020 | AAAGAUGACGCUUG | 1168 | AAAGAUGACGCUUGGUG | 1168 |
| 1446094.1 | gugucsasa | | aucususu | | GUGUGUCAG | | UGUCAG | |
| AD- | asusgac(Ghd)CfuUfGfGfugu | 872 | VPusGfscuga(C2p)acaccaAfgCfg | 1021 | AGAUGACGCUUGGU | 1169 | AGAUGACGCUUGGUGUG | 1169 |
| 1446095.1 | gucagscsa | | ucauscsu | | GUGUCAGCC | | UCAGCC | |
| AD- | gsascgc(Uhd)UfgGfUfGfugu | 873 | VPusCfsggcu(G2p)acacacCfaAfg | 1022 | AUGACGCUUGGUGU | 1170 | AUGACGCUUGGUGUGUC | 1170 |
| 1446096.1 | cagccsgsa | | cgucsasu | | GUCAGCCGU | | AGCCGU | |
| AD- | gscsugc(Chd)CfgGfUfUfgcu | 874 | VPusAfsagaGfaAfGfcaacCfgGfgc | 1023 | CUGCUGCCCGGUUG | 1171 | CUGCUGCCCGGUUGCUU | 1171 |
| 1446097.1 | ucucususa | | agcsasg | | CUUCUCUUU | | CUCUUU | |
| AD- | gsuscua(Ghd)CfaAfGfAfgca | 875 | VPusCfsacaCfcUfGfcucuUfgCfua | 1024 | GGGUCUAGCAAGAG | 1172 | GGGUCUAGCAAGAGCAG | 1172 |
| 1446098.1 | ggugusgsa | | gacscsc | | CAGGUGUGG | | GUGUGG | |
| AD- | gscsagg(Uhd)GfuGfGfGfuuu | 876 | VPusCfscucCfuAfAfacccAfcAfcc | 1025 | GAGCAGGUGUGGGU | 1173 | GAGCAGGUGUGGGUUUA | 1177 |
| 1446099.1 | aggagsgsa | | ugcsusc | | UUAGGAGAU | | GGAGGU | |
| AD- | csasggu(Ghd)UfgGfGfUfuua | 877 | VPusAfsccuc(C2p)uaaaccCfaCfac | 1026 | AGCAGGUGUGGGUU | 1174 | AGCAGGUGUGGGUUUAG | 1178 |
| 1446100.1 | ggaggsusa | | cugscsu | | UAGGAGAUA | | GAGGUG | |
| AD- | asgsgug(Uhd)GfgGfUfUfuag | 878 | VPusCfsaccUfcCfUfaaacCfcAfca | 1027 | GCAGGUGUGGGUUU | 1175 | GCAGGUGUGGGUUUAGG | 1179 |
| 1446101.1 | gaggusgsa | | ccusgsc | | AGGAGAUAU | | AGGUGU | |
| AD- | gsusgug(Ghd)GfuUfUfAfgga | 879 | VPusCfsacaCfcUfCfcuaaAfcCfca | 1028 | AGGUGUGGGUUUAG | 1176 | AGGUGUGGGUUUAGGAG | 1180 |
| 1446102.1 | ggugusgsa | | cacscsu | | GAGAUAUCU | | GUGUGU | |
| AD- | usgscuc(Uhd)CfaCfAfGfuacu | 880 | VPusCfsagcGfaGfUfacugUfgAfg | 1029 | | | CUUGCUCUCACAGUACU | 1181 |
| 1285244.1 | cgcusgsa | | agcasasg | | | | CGCUGA | |
| AD- | uscsuca(Chd)AfgUfAfCfucgc | 881 | VPusCfscuca(G2p)cgaguaCfuGfu | 1030 | | | GCUCUCACAGUACUCGC | 1182 |
| 1446103.1 | ugagsgsa | | gagasgsc | | | | UGAGGG | |
| AD- | uscsaca(Ghd)UfaCfUfCfgcug | 882 | VPusAfscccu(C2p)agcgagUfaCfu | 1031 | | | UCUCACAGUACUCGCUG | 1183 |
| 1446104.1 | agggsusa | | gugasgsa | | | | AGGGUG | |
| AD- | gscsuga(Ghd)GfgUfGfAfaca | 883 | VPusUfsuuuCfuUfGfuucaCfcCfu | 1032 | | | UCGCUGAGGGUGAACAA | 1184 |
| 1285235.1 | agaaasasa | | cagcsgsa | | | | GAAAAG | |
| AD- | asascaa(Ghd)AfaAfAfGfaccu | 884 | VPusUfsuauCfaGfGfucuuUfuCfu | 1033 | | | UGAACAAGAAAAGACCU | 264 |
| 1285238.1 | gauasasa | | uguuscsa | | | | GAUAAA | |
| AD- | asasgaa(Ahd)AfgAfCfCfugau | 885 | VPusUfscuuUfaUfCfagguCfuUfu | 1034 | | | ACAAGAAAAGACCUGAU | 1185 |
| 1285243.1 | aaagsasa | | ucuusgsu | | | | AAAGAU | |
| AD- | asgsaaa(Ahd)GfaCfCfUfgaua | 886 | VPusAfsucuUfuAfUfcaggUfcUfu | 1035 | | | CAAGAAAAGACCUGAUA | 1186 |
| 1285234.1 | aagasusa | | uucususg | | | | AAGAUU | |
| AD- | gsasaaa(Ghd)AfcCfUfGfauaa | 887 | VPusAfsaucUfuUfAfucagGfuCfu | 1036 | | | AAGAAAAGACCUGAUAA | 1187 |
| 1285239.1 | agaususa | | uuucsusu | | | | AGAUUA | |
| AD- | asasaag(Ahd)CfcUfGfAfuaaa | 888 | VPusUfsaauCfuUfUfaucaGfgUfc | 1037 | | | AGAAAAGACCUGAUAAA | 1188 |
| 1285232.1 | gauusasa | | uuuuscsu | | | | GAUUAA | |
| AD- | asasaga(Chd)CfuGfAfUfaaag | 889 | VPusUfsuaaUfcUfUfuaucAfgGfu | 1038 | | | GAAAAGACCUGAUAAAG | 1189 |
| 1285231.1 | auuasasa | | cuuususc | | | | AUUAAC | |
| AD- | asasgac(Chd)UfgAfUfAfaaga | 890 | VPusGfsuuaAfuCfUfuuauCfaGfg | 1039 | | | AAAAGACCUGAUAAAGA | 1190 |
| 1285240.1 | uuaascsa | | ucuususu | | | | UUAACC | |
| AD- | gsasccu(Ghd)AfuAfAfAfgau | 891 | VPusUfsgguUfaAfUfcuuuAfuCfa | 1040 | | | AAGACCUGAUAAAGAUU | 1191 |
| 1285241.1 | uaaccsasa | | ggucsusu | | | | AACCAG | |
| AD- | ascscug(Ahd)UfaAfAfGfauu | 892 | VPusCfsuggUfuAfAfucuuUfaUfc | 1041 | | | AGACCUGAUAAAGAUUA | 1192 |
| 1285242.1 | aaccasgsa | | agguscsu | | | | ACCAGA | |
| AD- | asasaga(Uhd)UfaAfCfCfagaa | 893 | VPusUfsuuuCfuUfCfugguUfaAfu | 1042 | | | AUAAAGAUUAACCAGAA | 1193 |
| 1285233.1 | gaaasasa | | cuuusasu | | | | GAAAAC | |
| AD- | asusuaa(Chd)CfaGfAfAfgaaa | 894 | VPusCfsuugUfuUfUfcuucUfgGfu | 1043 | | | AGAUUAACCAGAAGAAA | 1194 |
| 1285237.1 | acaasgsa | | uaauscsu | | | | ACAAGG | |
| AD- | asascca(Ghd)AfaGfAfAfaaca | 895 | VPusCfsuccUfuGfUfuuucUfuCfu | 1044 | | | UUAACCAGAAGAAAACA | 1195 |
| 1285236.1 | aggasgsa | | gguusasa | | | | AGGAGG | |
| AD- | gsgsagg(Ghd)AfaAfCfAfacc | 896 | VPusGfsgcug(C2p)gguuguUfuCfc | 1045 | | | AAGGAGGGAAACAACCG | 1196 |
| 1446105.1 | gcagcscsa | | cuccsusu | | | | CAGCCU | |
| AD- | gsasggg(Ahd)AfaCfAfAfccg | 897 | VPusAfsggcu(G2p)cgguugUfuUf | 1046 | | | AGGAGGGAAACAACCGC | 1197 |
| 1446106.1 | cagccsusa | | cccucscsu | | | | AGCCUG | |
| AD- | csasgcc(Uhd)GfuAfGfCfaagc | 898 | VPusCfscaga(G2p)cuugcuAfcAfg | 1047 | | | CGCAGCCUGUAGCAAGC | 1198 |
| 1446107.1 | ucugsgsa | | gcugscsg | | | | UCUGGA | |
| AD- | csuscug(Ghd)AfaCfUfCfagga | 899 | VPusGfscgac(Tgn)ccugagUfuCfc | 1048 | | | AGCUCUGGAACUCAGGA | 1199 |
| 1446108.1 | gucgscsa | | agagscsu | | | | GUCGCG | |
| AD- | uscsucc(Uhd)CfaGfAfGfcucg | 900 | VPusUfsgcgu(C2p)gagcucUfgAfg | 1049 | | | GCUCUCCUCAGAGCUCG | 1200 |
| 1446109.1 | acgcsasa | | gagasgsc | | | | ACGCAU | |
| AD- | uscscuc(Ahd)GfaGfCfUfcgac | 901 | VPusAfsaugc(G2p)ucgagcUfcUfg | 1050 | | | UCUCCUCAGAGCUCGAC | 1201 |
| 1446110.1 | gcaususa | | aggasgsa | | | | GCAUUU | |
| AD- | ascsuuu(Chd)CfcUfCfUfcauu | 902 | VPusAfsgagAfaAfUfgagaGfgGfa | 1051 | | | UUACUUUCCCUCUCAUU | 1202 |
| 1446111.1 | ucucsusa | | aagusasa | | | | UCUCUG | |
| AD- | csusuuc(Chd)CfuCfUfCfauuu | 903 | VPusCfsagaGfaAfAfugagAfgGfg | 1052 | | | UACUUUCCCUCUCAUUU | 1203 |
| 1446112.1 | cucusgsa | | aaagsusa | | | | CUCUGA | |
| AD- | ususucc(Chd)UfcUfCfAfuuu | 904 | VPusUfscaga(G2p)aaaugaGfaGfg | 1053 | | | ACUUUCCCUCUCAUUUC | 1204 |
| 1446113.1 | cucugsasa | | gaaasgsu | | | | UCUGAC | |
| AD- | uscsccu(Chd)UfcAfUfUfucuc | 905 | VPusGfsguca(G2p)agaaauGfaGfa | 1054 | | | UUUCCCUCUCAUUUCUC | 1205 |
| 1446114.1 | ugacscsa | | gggasasa | | | | UGACCG | |
| AD- | cscscuc(Uhd)CfaUfUfUfcucu | 906 | VPusCfsgguc(Agn)gagaaaUfgAfg | 1055 | | | UUCCCUCUCAUUUCUCU | 208 |
| 1446115.1 | gaccsgsa | | agggsasa | | | | GACCGA | |
| AD- | cscsucu(Chd)AfuUfUfCfucu | 907 | VPusUfscggu(C2p)agagaaAfuGfa | 1056 | | | UCCCUCUCAUUUCUCUG | 1206 |
| 1446116.1 | gaccgsasa | | gaggsgsa | | | | ACCGAA | |
| AD- | uscsuca(Uhd)UfuCfUfCfugac | 908 | VPusCfsuucg(G2p)ucagagAfaAfu | 1057 | | | CCUCUCAUUUCUCUGAC | 1207 |
| 1446117.1 | cgaasgsa | | gagasgsg | | | | CGAAGC | |
| AD- | csuscau(Uhd)UfcUfCfUfgacc | 909 | VPusGfscuuc(G2p)gucagaGfaAfa | 1058 | | | CUCUCAUUUCUCUGACC | 1208 |
| 1446118.1 | gaagscsa | | ugagsasg | | | | GAAGCU | |
| AD- | uscsauu(Uhd)CfuCfUfGfaccg | 910 | VPusAfsgcuu(C2p)ggucagAfgAfa | 1059 | | | UCUCAUUUCUCUGACCG | 206 |
| 1446119.1 | aagcsusa | | augasgsa | | | | AAGCUG | |
| AD- | csuscug(Ahd)CfcGfAfAfgcu | 911 | VPusAfscacc(C2p)agcuucGfgUfc | 1060 | | | UUCUCUGACCGAAGCUG | 203 |
| 1446120.1 | gggugsusa | | agagsasa | | | | GGUGUC | |
| AD- | gsgsugu(Chd)GfgGfCfUfuuc | 912 | VPusAfsgagg(C2p)gaaagcCfcGfa | 1061 | | | UGGGUGUCGGGCUUUCG | 1209 |
| 1446121.1 | gccucsusa | | caccscsa | | | | CCUCUA | |
| AD- | uscsggg(Chd)UfuUfCfGfccu | 913 | VPusCfsgcuAfgAfGfgcgaAfaGfc | 1062 | | | UGUCGGGCUUUCGCCUC | 1210 |
| 1446122.1 | cuagcsgsa | | ccgascsa | | | | UAGCGA | |
| AD- | csusuuc(Ghd)CfcUfCfUfagcg | 914 | VPusCfscagu(C2p)gcuagaGfgCfg | 1063 | | | GGCUUUCGCCUCUAGCG | 1211 |
| 1446123.1 | acugsgsa | | aaagscsc | | | | ACUGGU | |
| AD- | ususcgc(Chd)UfcUfAfGfcgac | 915 | VPusCfsaccAfgUfCfgcuaGfaGfgc | 1064 | | | CUUUCGCCUCUAGCGAC | 1212 |
| 1446124.1 | uggusgsa | | gaasasg | | | | UGGUGG | |
| AD- | uscsgcc(Uhd)CfuAfGfCfgacu | 916 | VPusCfscacCfaGfUfcgcuAfgAfg | 1065 | | | UUUCGCCUCUAGCGACU | 201 |
| 1446125.1 | ggugsgsa | | gcgasasa | | | | GGUGGA | |
| AD- | csgsccu(Chd)UfaGfCfGfacug | 917 | VPusUfsccac(C2p)agucgcUfaGfa | 1066 | | | UUCGCCUCUAGCGACUG | 1213 |
| 1446126.1 | guggsasa | | ggcgsasa | | | | GUGGAA | |
| AD- | gscscuc(Uhd)AfgCfGfAfcug | 918 | VPusUfsucca(C2p)cagucgCfuAfg | 1067 | | | UCGCCUCUAGCGACUGG | 1214 |
| 1446127.1 | guggasasa | | aggcsgsa | | | | UGGAAU | |
| AD- | csuscua(Ghd)CfgAfCfUfggu | 919 | VPusAfsauuc(C2p)accaguCfgCfu | 1068 | | | GCCUCUAGCGACUGGUG | 1215 |
| 1446128.1 | ggaaususa | | agagsgsc | | | | GAAUUG | |
| AD- | uscsuag(Chd)GfaCfUfGfgug | 920 | VPusCfsaauu(C2p)caccagUfcGfc | 1069 | | | CCUCUAGCGACUGGUGG | 1216 |
| 1446129.1 | gaauusgsa | | uagasgsg | | | | AAUUGC | |
| AD- | gsascug(Ghd)UfgGfAfAfuug | 921 | VPusUfsgcag(G2p)caauucCfaCfc | 1070 | | | GCGACUGGUGGAAUUGC | 1217 |
| 1446130.1 | ccugcsasa | | agucsgsc | | | | CUGCAU | |
| AD- | ascsugg(Uhd)GfgAfAfUfugc | 922 | VPusAfsugca(G2p)gcaauuCfcAfc | 1071 | | | CGACUGGUGGAAUUGCC | 1218 |
| 1446131.1 | cugcasusa | | caguscsg | | | | UGCAUC | |
| AD- | usgsgug(Ghd)AfaUfUfGfccu | 923 | VPusGfsgaug(C2p)aggcaaUfuCfc | 1072 | | | ACUGGUGGAAUUGCCUG | 1219 |
| 1446132.1 | gcaucscsa | | accasgsu | | | | CAUCCG | |
| AD- | uscsugg(Chd)CfuCfUfUfccu | 924 | VPusAfsaagCfaAfGfgaagAfgGfc | 1073 | | | GAUCUGGCCUCUUCCUU | 1220 |
| 1446134.1 | ugcuususa | | cagasusc | | | | GCUUUC | |
| AD- | csusggc(Chd)UfcUfUfCfcuu | 925 | VPusGfsaaag(C2p)aaggaaGfaGfg | 1074 | | | AUCUGGCCUCUUCCUUG | 1221 |
| 1446135.1 | gcuuuscsa | | ccagsasu | | | | CUUUCC | |
| AD- | usgsgcc(Uhd)CfuUfCfCfuug | 926 | VPusGfsgaaa(G2p)caaggaAfgAfg | 1075 | | | UCUGGCCUCUUCCUUGC | 1222 |
| 1446136.1 | cuuucscsa | | gccasgsa | | | | UUUCCC | |
| AD- | gsgsccu(Chd)UfuCfCfUfugc | 927 | VPusGfsggaAfaGfCfaaggAfaGfa | 1076 | | | CUGGCCUCUUCCUUGCU | 1223 |
| 1446137.1 | uuuccscsa | | ggccsasg | | | | UUCCCG | |
| AD- | gscscuc(Uhd)UfcCfUfUfgcu | 928 | VPusCfsgggAfaAfGfcaagGfaAfg | 1077 | | | UGGCCUCUUCCUUGCUU | 1224 |
| 1446138.1 | uucccsgsa | | aggcscsa | | | | UCCCGC | |
| AD- | ususccu(Uhd)GfcUfUfUfccc | 929 | VPusGfsaggg(C2p)gggaaaGfcAfa | 1078 | | | UCUUCCUUGCUUUCCCG | 1225 |
| 1446139.1 | gcccuscsa | | ggaasgsa | | | | CCCUCA | |
| AD- | uscscuu(Ghd)CfuUfUfCfccgc | 930 | VPusUfsgagg(G2p)cgggaaAfgCfa | 1079 | | | CUUCCUUGCUUUCCCGC | 1226 |
| 1446140.1 | ccucsasa | | aggasasg | | | | CCUCAG | |
| AD- | cscsuug(Chd)UfuUfCfCfcgcc | 931 | VPusCfsugag(G2p)gcgggaAfaGfc | 1080 | | | UUCCUUGCUUUCCCGCC | 1227 |
| 1446141.1 | cucasgsa | | aaggsasa | | | | CUCAGU | |
| AD- | csusugc(Uhd)UfuCfCfCfgccc | 932 | VPusAfscuga(G2p)ggcgggAfaAfg | 1081 | | | UCCUUGCUUUCCCGCCC | 1228 |
| 1446142.1 | ucagsusa | | caagsgsa | | | | UCAGUA | |
| AD- | asgsuac(Chd)CfgAfGfCfuguc | 933 | VPusAfsagga(G2p)acagcuCfgGfg | 1082 | | | UCAGUACCCGAGCUGUC | 1229 |
| 1446143.1 | uccususa | | uacusgsa | | | | UCCUUC | |
| AD- | usasccc(Ghd)AfgCfUfGfucuc | 934 | VPusGfsgaag(G2p)agacagCfuCfg | 1083 | | | AGUACCCGAGCUGUCUC | 1230 |
| 1446144.1 | cuucscsa | | gguascsu | | | | CUUCCC | |
| AD- | gsasgga(Ghd)AfuCfAfUfgcg | 935 | VPusUfscauc(C2p)cgcaugAfuCfu | 1084 | | | GCGAGGAGAUCAUGCGG | 1231 |
| 1446145.1 | ggaugsasa | | ccucsgsc | | | | GAUGAG | |
| AD- | asgsacg(Chd)CfuGfCfAfcaau | 936 | VPusCfsugaAfaUfUfgugcAfgGfc | 1085 | | | GGAGACGCCUGCACAAU | 1232 |
| 1446146.1 | uucasgsa | | gucuscsc | | | | UUCAGC | |
| AD- | gsascgc(Chd)UfgCfAfCfaauu | 937 | VPusGfscugAfaAfUfugugCfaGfg | 1086 | | | GAGACGCCUGCACAAUU | 1233 |
| 1446147.1 | ucagscsa | | cgucsusc | | | | UCAGCC | |
| AD- | csgsccu(Ghd)CfaCfAfAfuuuc | 938 | VPusGfsggcu(G2p)aaauugUfgCfa | 1087 | | | GACGCCUGCACAAUUUC | 1234 |
| 1446148.1 | agccscsa | | ggcgsusc | | | | AGCCCA | |
| AD- | cscsugc(Ahd)CfaAfUfUfucag | 939 | VPusUfsuggg(C2p)ugaaauUfgUfg | 1088 | | | CGCCUGCACAAUUUCAG | 1235 |
| 1446149.1 | cccasasa | | caggscsg | | | | CCCAAG | |
| AD- | csusgca(Chd)AfaUfUfUfcagc | 940 | VPusCfsuugg(G2p)cugaaaUfuGfu | 1089 | | | GCCUGCACAAUUUCAGC | 1236 |
| 1446150.1 | ccaasgsa | | gcagsgsc | | | | CCAAGC | |
| AD- | usgscac(Ahd)AfuUfUfCfagcc | 941 | VPusGfscuug(G2p)gcugaaAfuUfg | 1090 | | | CCUGCACAAUUUCAGCC | 1237 |
| 1446151.1 | caagscsa | | ugcasgsg | | | | CAAGCU | |
| AD- | csasauu(Uhd)CfaGfCfCfcaag | 942 | VPusAfsgaag(C2p)uugggcUfgAfa | 1091 | | | CACAAUUUCAGCCCAAG | 1238 |
| 1446152.1 | cuucsusa | | auugsusg | | | | CUUCUA | |
| AD- | asasuuu(Chd)AfgCfCfCfaagc | 943 | VPusUfsagaa(G2p)cuugggCfuGfa | 1092 | | | ACAAUUUCAGCCCAAGC | 1239 |
| 1446153.1 | uucusasa | | aauusgsu | | | | UUCUAG | |
| AD- | csasgcc(Chd)AfaGfCfUfucua | 944 | VPusCfsucuc(Tgn)agaagcUfuGfg | 1093 | | | UUCAGCCCAAGCUUCUA | 1240 |
| 1446154.1 | gagasgsa | | gcugsasa | | | | GAGAGU | |
| AD- | asgsccc(Ahd)AfgCfUfUfcuag | 945 | VPusAfscucu(C2p)uagaagCfuUfg | 1094 | | | UCAGCCCAAGCUUCUAG | 1241 |
| 1446155.1 | agagsusa | | ggcusgsa | | | | AGAGUG | |
| AD- | gscscca(Ahd)GfcUfUfCfuaga | 946 | VPusCfsacuc(Tgn)cuagaaGfcUfu | 1095 | | | CAGCCCAAGCUUCUAGA | 1242 |
| 1446156.1 | gagusgsa | | gggcsusg | | | | GAGUGG | |
| AD- | cscscaa(Ghd)CfuUfCfUfagag | 947 | VPusCfscacUfcUfCfuagaAfgCfuu | 1096 | | | AGCCCAAGCUUCUAGAG | 1243 |
| 1446157.1 | agugsgsa | | gggscsu | | | | AGUGGU | |
| AD- | cscsaag(Chd)UfuCfUfAfgaga | 948 | VPusAfsccac(Tgn)cucuagAfaGfc | 1097 | | | GCCCAAGCUUCUAGAGA | 1244 |
| 1446158.1 | guggsusa | | uuggsgsc | | | | GUGGUG | |
| AD- | csasagc(Uhd)UfcUfAfGfagag | 949 | VPusCfsaccAfcUfCfucuaGfaAfgc | 1098 | | | CCCAAGCUUCUAGAGAG | 1245 |
| 1446159.1 | uggusgsa | | uugsgsg | | | | UGGUGA | |
| AD- | asasgcu(Uhd)CfuAfGfAfgag | 950 | VPusUfscacc(Agn)cucucuAfgAfa | 1099 | | | CCAAGCUUCUAGAGAGU | 1246 |
| 1446160.1 | uggugsasa | | gcuusgsg | | | | GGUGAU | |
| AD- | asgscuu(Chd)UfaGfAfGfagu | 951 | VPusAfsucac(C2p)acucucUfaGfa | 1100 | | | CAAGCUUCUAGAGAGUG | 1247 |
| 1446161.1 | ggugasusa | | agcususg | | | | GUGAUG | |
| AD- | gscsuuc(Uhd)AfgAfGfAfgug | 952 | VPusCfsauca(C2p)cacucuCfuAfg | 1101 | | | AAGCUUCUAGAGAGUGG | 1248 |
| 1446162.1 | gugausgsa | | aagcsusu | | | | UGAUGA | |
| AD- | ususcua(Ghd)AfgAfGfUfggu | 953 | VPusGfsucau(C2p)accacuCfuCfu | 1102 | | | GCUUCUAGAGAGUGGUG | 1249 |
| 1446163.1 | gaugascsa | | agaasgsc | | | | AUGACU | |
| AD- | usasgag(Ahd)GfuGfGfUfgau | 954 | VPusCfsaagu(C2p)aucaccAfcUfc | 1103 | | | UCUAGAGAGUGGUGAUG | 1250 |
| 1446164.1 | gacuusgsa | | ucuasgsa | | | | ACUUGC | |
| AD- | asgsaga(Ghd)UfgGfUfGfaug | 955 | VPusGfscaag(Tgn)caucacCfaCfuc | 1104 | | | CUAGAGAGUGGUGAUGA | 1251 |
| 1446165.1 | acuugscsa | | ucusasg | | | | CUUGCA | |
| AD- | gsasgag(Uhd)GfgUfGfAfuga | 956 | VPusUfsgcaa(G2p)ucaucaCfcAfc | 1105 | | | UAGAGAGUGGUGAUGAC | 1252 |
| 1446166.1 | cuugcsasa | | ucucsusa | | | | UUGCAU | |
| AD- | asgsugg(Uhd)GfaUfGfAfcuu | 957 | VPusAfsuaug(C2p)aagucaUfcAfc | 1106 | | | AGAGUGGUGAUGACUUG | 1253 |
| 1446167.1 | gcauasusa | | cacuscsu | | | | CAUAUG | |
| AD- | gsgsuga(Uhd)GfaCfUfUfgca | 958 | VPusCfsucaUfaUfGfcaagUfcAfuc | 1107 | | | GUGGUGAUGACUUGCAU | 1254 |
| 1446168.1 | uaugasgsa | | accsasc | | | | AUGAGG | |
| AD- | gsusgau(Ghd)AfcUfUfGfcau | 959 | VPusCfscucAfuAfUfgcaaGfuCfa | 1108 | | | UGGUGAUGACUUGCAUA | 1255 |
| 1446169.1 | augagsgsa | | ucacscsa | | | | UGAGGG | |
| AD- | usgsaug(Ahd)CfuUfGfCfaua | 960 | VPusCfsccuCfaUfAfugcaAfgUfca | 1109 | | | GGUGAUGACUUGCAUAU | 1256 |
| 1446170.1 | ugaggsgsa | | ucascsc | | | | GAGGGC | |
| AD- | asgsggc(Ahd)GfcAfAfUfgca | 961 | VPusCfscgaCfuUfGfcauuGfcUfg | 1110 | | | UGAGGGCAGCAAUGCAA | 1257 |
| 1446171.1 | agucgsgsa | | cccuscsa | | | | GUCGGU | |
| AD- | gsgsgca(Ghd)CfaAfUfGfcaag | 962 | VPusAfsccgAfcUfUfgcauUfgCfu | 1111 | | | GAGGGCAGCAAUGCAAG | 1258 |
| 1446172.1 | ucggsusa | | gcccsusc | | | | UCGGUG | |
| AD- | gsgscag(Chd)AfaUfGfCfaagu | 963 | VPusCfsaccGfaCfUfugcaUfuGfcu | 1112 | | | AGGGCAGCAAUGCAAGU | 1259 |
| 1446173.1 | cggusgsa | | gccscsu | | | | CGGUGU | |
| AD- | gscsagc(Ahd)AfuGfCfAfagu | 964 | VPusAfscacCfgAfCfuugcAfuUfg | 1113 | | | GGGCAGCAAUGCAAGUC | 1260 |
| 1446174.1 | cggugsusa | | cugcscsc | | | | GGUGUG | |
| AD- | csasgca(Ahd)UfgCfAfAfguc | 965 | VPusCfsacaCfcGfAfcuugCfaUfug | 1114 | | | GGCAGCAAUGCAAGUCG | 1261 |
| 1446175.1 | ggugusgsa | | cugscsc | | | | GUGUGC | |
| AD- | csasaug(Chd)AfaGfUfCfggu | 966 | VPusGfsagca(C2p)accgacUfuGfc | 1115 | | | AGCAAUGCAAGUCGGUG | 1262 |
| 1446176.1 | gugcuscsa | | auugscsu | | | | UGCUCC | |
| AD- | csusgug(Ghd)GfaCfAfUfgac | 967 | VPusAfsacca(G2p)gucaugUfcCfc | 1116 | | | UUCUGUGGGACAUGACC | 1263 |
| 1446177.1 | cuggususa | | acagsasa | | | | UGGUUG | |
| AD- | usgsugg(Ghd)AfcAfUfGfacc | 968 | VPusCfsaacCfaGfGfucauGfuCfcc | 1117 | | | UCUGUGGGACAUGACCU | 1264 |
| 1446178.1 | ugguusgsa | | acasgsa | | | | GGUUGC | |
| AD- | gsusggg(Ahd)CfaUfGfAfccu | 969 | VPusGfscaac(C2p)aggucaUfgUfc | 1118 | | | CUGUGGGACAUGACCUG | 1265 |
| 1446179.1 | gguugscsa | | ccacsasg | | | | GUUGCU | |
| AD- | usgsgga(Chd)AfuGfAfCfcug | 970 | VPusAfsgcaAfcCfAfggucAfuGfu | 1119 | | | UGUGGGACAUGACCUGG | 1266 |
| 1446180.1 | guugcsusa | | cccascsa | | | | UUGCUU | |
| AD- | gsascau(Ghd)AfcCfUfGfguu | 971 | VPusUfsgaag(C2p)aaccagGfuCfa | 1120 | | | GGGACAUGACCUGGUUG | 1267 |
| 1446181.1 | gcuucsasa | | ugucscsc | | | | CUUCAC | |
| AD- | ascsaug(Ahd)CfcUfGfGfuug | 972 | VPusGfsugaa(G2p)caaccaGfgUfc | 1121 | | | GGACAUGACCUGGUUGC | 1268 |
| 1446182.1 | cuucascsa | | auguscsc | | | | UUCACA | |
| AD- | usgsacc(Uhd)GfgUfUfGfcuu | 973 | VPusGfscugu(G2p)aagcaaCfcAfg | 1122 | | | CAUGACCUGGUUGCUUC | 1269 |
| 1446183.1 | cacagscsa | | gucasusg | | | | ACAGCU | |
| AD- | gsasccu(Ghd)GfuUfGfCfuuc | 974 | VPusAfsgcug(Tgn)gaagcaAfcCfa | 1123 | | | AUGACCUGGUUGCUUCA | 1270 |
| 1446184.1 | acagcsusa | | ggucsasu | | | | CAGCUC | |
| AD- | ascscug(Ghd)UfuGfCfUfucac | 975 | VPusGfsagcu(G2p)ugaagcAfaCfc | 1124 | | | UGACCUGGUUGCUUCAC | 1271 |
| 1446185.1 | agcuscsa | | agguscsa | | | | AGCUCC | |
| AD- | cscsugg(Uhd)UfgCfUfUfcaca | 976 | VPusGfsgagc(Tgn)gugaagCfaAfc | 1125 | | | GACCUGGUUGCUUCACA | 1272 |
| 1446186.1 | gcucscsa | | caggsusc | | | | GCUCCG | |
| AD- | csusggu(Uhd)GfcUfUfCfaca | 977 | VPusCfsggag(C2p)ugugaaGfcAfa | 1126 | | | ACCUGGUUGCUUCACAG | 1273 |
| 1446187.1 | gcuccsgsa | | ccagsgsu | | | | CUCCGA | |
| AD- | usgsguu(Ghd)CfuUfCfAfcag | 978 | VPusUfscgga(G2p)cugugaAfgCfa | 1127 | | | CCUGGUUGCUUCACAGC | 1274 |
| 1446188.1 | cuccgsasa | | accasgsg | | | | UCCGAG | |
| AD- | gsgsuug(Chd)UfuCfAfCfagc | 979 | VPusCfsucgGfaGfCfugugAfaGfc | 1128 | | | CUGGUUGCUUCACAGCU | 1275 |
| 1446189.1 | uccgasgsa | | aaccsasg | | | | CCGAGA | |
| AD- | gsusugc(Uhd)UfcAfCfAfgcu | 980 | VPusUfscucg(G2p)agcuguGfaAfg | 1129 | | | UGGUUGCUUCACAGCUC | 1276 |
| 1446190.1 | ccgagsasa | | caacscsa | | | | CGAGAU | |
| AD- | ususgcu(Uhd)CfaCfAfGfcucc | 981 | VPusAfsucuc(G2p)gagcugUfgAfa | 1130 | | | GGUUGCUUCACAGCUCC | 1277 |
| 1446191.1 | gagasusa | | gcaascsc | | | | GAGAUG | |
| AD- | csasgcu(Chd)CfgAfGfAfugac | 982 | VPusUfscugu(G2p)ucaucuCfgGfa | 1131 | | | CACAGCUCCGAGAUGAC | 1278 |
| 1446192.1 | acagsasa | | gcugsusg | | | | ACAGAC | |
| AD- | gscsucc(Ghd)AfgAfUfGfacac | 983 | VPusAfsgucu(G2p)ugucauCfuCfg | 1132 | | | CAGCUCCGAGAUGACAC | 1279 |
| 1446193.1 | agacsusa | | gagcsusg | | | | AGACUU | |
| AD- | csusccg(Ahd)GfaUfGfAfcaca | 984 | VPusAfsaguc(Tgn)gugucaUfcUfc | 1133 | | | AGCUCCGAGAUGACACA | 1280 |
| 1446194.1 | gacususa | | ggagscsu | | | | GACUUG | |
| AD- | uscscga(Ghd)AfuGfAfCfacag | 985 | VPusCfsaagu(C2p)ugugucAfuCfu | 1134 | | | GCUCCGAGAUGACACAG | 1281 |
| 1446195.1 | acuusgsa | | cggasgsc | | | | ACUUGC | |
| AD- | cscsgag(Ahd)UfgAfCfAfcaga | 986 | VPusGfscaag(Tgn)cuguguCfaUfc | 1135 | | | CUCCGAGAUGACACAGA | 1282 |
| 1446196.1 | cuugscsa | | ucggsasg | | | | CUUGCU | |
| AD- | csgsaga(Uhd)GfaCfAfCfagac | 987 | VPusAfsgcaa(G2p)ucugugUfcAfu | 1136 | | | UCCGAGAUGACACAGAC | 1283 |
| 1446197.1 | uugcsusa | | cucgsgsa | | | | UUGCUU | |
| AD- | gsasgau(Ghd)AfcAfCfAfgac | 988 | VPusAfsagca(Agn)gucuguGfuCfa | 1137 | | | CCGAGAUGACACAGACU | 1284 |
| 1446198.1 | uugcususa | | ucucsgsg | | | | UGCUUA | |
| AD- | gsasuga(Chd)AfcAfGfAfcuu | 989 | VPusUfsuaag(C2p)aagucuGfuGfu | 1138 | | | GAGAUGACACAGACUUG | 1285 |
| 1446199.1 | gcuuasasa | | caucsusc | | | | CUUAAA | |
| AD- | asusgac(Ahd)CfaGfAfCfuugc | 990 | VPusUfsuuaa(G2p)caagucUfgUfg | 1139 | | | AGAUGACACAGACUUGC | 1286 |
| 1446200.1 | uuaasasa | | ucauscsu | | | | UUAAAG | |
| AD- | usgsaca(Chd)AfgAfCfUfugc | 991 | VPusCfsuuuAfaGfCfaaguCfuGfu | 1140 | | | GAUGACACAGACUUGCU | 1287 |
| 1446201.1 | uuaaasgsa | | gucasusc | | | | UAAAGG | |
| AD- | gsascac(Ahd)GfaCfUfUfgcuu | 992 | VPusCfscuuUfaAfGfcaagUfcUfg | 1141 | | | AUGACACAGACUUGCUU | 1288 |
| 1446202.1 | aaagsgsa | | ugucsasu | | | | AAAGGA | |
| AD- | ascsaca(Ghd)AfcUfUfGfcuua | 993 | VPusUfsccuUfuAfAfgcaaGfuCfu | 1142 | | | UGACACAGACUUGCUUA | 1289 |
| 1446203.1 | aaggsasa | | guguscsa | | | | AAGGAA | |
| AD- | csasgac(Uhd)UfgCfUfUfaaag | 994 | VPusAfscuuc(C2p)uuuaagCfaAfg | 1143 | | | CACAGACUUGCUUAAAG | 1290 |
| 1446204.1 | gaagsusa | | ucugsusg | | | | GAAGUG | |
| AD- | asgsacu(Uhd)GfcUfUfAfaag | 995 | VPusCfsacuu(C2p)cuuuaaGfcAfa | 1144 | | | ACAGACUUGCUUAAAGG | 1291 |
| 1446205.1 | gaagusgsa | | gucusgsu | | | | AAGUGA | |

TABLE 7
Single Dose Screen of dsRNA Agents Targeting the Sense Strand of Either
Exon 1A or the 3′-side of the Intronic Repeat Between Exons 1A and 1B

| | 10 nM % | | 1 nM % | | 0.1 nM % | |
|---|---|---|---|---|---|---|
| | Message | | Message | | Message | |
| Sample Name | Remaining | STDEV | Remaining | STDEV | Remaining | STDEV |
| AD-1285232.1 | 9 | 1 | 11 | 1 | 17 | 3 |
| AD-1446196.1 | 9 | 1 | 10 | 1 | 16 | 3 |
| AD-1446111.1 | 9 | 2 | 10 | 2 | 16 | 1 |
| AD-1446182.1 | 10 | 1 | 12 | 4 | 24 | 1 |
| AD-1446084.1 | 10 | 2 | 10 | 1 | 19 | 4 |
| AD-1285231.1 | 10 | 2 | 12 | 1 | 15 | 1 |
| AD-1446185.1 | 10 | 2 | 15 | 1 | 22 | 3 |
| AD-1446200.1 | 10 | 1 | 15 | 3 | 18 | 2 |
| AD-1446083.1 | 11 | 1 | 18 | 6 | 24 | 2 |
| AD-1285233.1 | 11 | 3 | 15 | 3 | 21 | 3 |
| AD-1446202.1 | 11 | 2 | 14 | 2 | 18 | 4 |
| AD-1446152.1 | 12 | 1 | 14 | 2 | 22 | 4 |
| AD-1446188.1 | 12 | 1 | 13 | 1 | 18 | 1 |
| AD-1285241.1 | 12 | 2 | 12 | 4 | 20 | 5 |
| AD-1446113.1 | 12 | 2 | 21 | 4 | 29 | 8 |
| AD-1446150.1 | 12 | 2 | 20 | 1 | 30 | 3 |
| AD-1446090.1 | 13 | 2 | 17 | 4 | 24 | 3 |
| AD-1446197.1 | 13 | 3 | 11 | 2 | 19 | 2 |
| AD-1446194.1 | 14 | 3 | 17 | 1 | 27 | 2 |
| AD-1446184.1 | 14 | 1 | 17 | 4 | 24 | 3 |
| AD-1446199.1 | 14 | 2 | 20 | 4 | 23 | 6 |
| AD-1285237.1 | 16 | 5 | 17 | 1 | 23 | 3 |
| AD-1285242.1 | 16 | 2 | 16 | 2 | 20 | 5 |
| AD-1285234.1 | 17 | 3 | 18 | 2 | 23 | 4 |
| AD-1446087.1 | 17 | 1 | 24 | 3 | 32 | 6 |
| AD-1446112.1 | 17 | 3 | 15 | 7 | 20 | 7 |
| AD-1446103.1 | 17 | 2 | 27 | 7 | 40 | 5 |
| AD-1285238.1 | 17 | 3 | 18 | 3 | 23 | 6 |
| AD-1446156.1 | 17 | 3 | 21 | 2 | 33 | 4 |
| AD-1446161.1 | 17 | 1 | 15 | 5 | 25 | 4 |
| AD-1446114.1 | 18 | 5 | 22 | 3 | 37 | 8 |
| AD-1446154.1 | 18 | 2 | 21 | 4 | 32 | 5 |
| AD-1285239.1 | 18 | 7 | 16 | 2 | 21 | 1 |
| AD-1285236.1 | 18 | 4 | 25 | 2 | 25 | 7 |
| AD-1446166.1 | 18 | 1 | 17 | 2 | 37 | 5 |
| AD-1446180.1 | 18 | 5 | 25 | 4 | 36 | 9 |
| AD-1446192.1 | 18 | 2 | 19 | 2 | 29 | 4 |
| AD-1446095.1 | 18 | 4 | 20 | 1 | 31 | 1 |
| AD-1285243.1 | 18 | 4 | 18 | 4 | 24 | 2 |
| AD-1446167.1 | 18 | 2 | 22 | 4 | 40 | 7 |
| AD-1285244.1 | 19 | 3 | 19 | 3 | 31 | 3 |
| AD-1446158.1 | 19 | 3 | 20 | 2 | 30 | 1 |
| AD-1446201.1 | 19 | 4 | 23 | 0 | 24 | 2 |
| AD-1446168.1 | 19 | 3 | 23 | 3 | 44 | 9 |
| AD-1446092.1 | 20 | 5 | 20 | 3 | 27 | 4 |
| AD-1446157.1 | 20 | 3 | 32 | 3 | 41 | 9 |
| AD-1446170.1 | 21 | 3 | 25 | 2 | 52 | 3 |
| AD-1446149.1 | 22 | 2 | 27 | 6 | 37 | 4 |
| AD-1446091.1 | 22 | 5 | 26 | 6 | 41 | 4 |
| AD-1446198.1 | 22 | 3 | 23 | 2 | 41 | 7 |
| AD-1446117.1 | 23 | 4 | 26 | 3 | 26 | 4 |
| AD-1446073.1 | 23 | 3 | 27 | 2 | 37 | 3 |
| AD-1446148.1 | 23 | 3 | 33 | 2 | 42 | 5 |
| AD-1446088.1 | 23 | 3 | 21 | 5 | 33 | 4 |
| AD-1446160.1 | 23 | 4 | 23 | 3 | 30 | 6 |
| AD-1446183.1 | 23 | 6 | 24 | 1 | 40 | 7 |
| AD-1446153.1 | 24 | 4 | 23 | 1 | 35 | 3 |
| AD-1446147.1 | 24 | 2 | 36 | 6 | 41 | 3 |
| AD-1285240.1 | 24 | 3 | 22 | 1 | 25 | 3 |
| AD-1446089.1 | 24 | 1 | 23 | 5 | 37 | 6 |
| AD-1446205.1 | 24 | 10 | 26 | 5 | 31 | 3 |
| AD-1446195.1 | 25 | 3 | 28 | 5 | 45 | 4 |
| AD-1446162.1 | 25 | 1 | 26 | 2 | 33 | 6 |
| AD-1285246.2 | 25 | 8 | 26 | 4 | 34 | 6 |
| AD-1446177.1 | 25 | 3 | 27 | 6 | 46 | 4 |
| AD-1446189.1 | 26 | 5 | 27 | 5 | 42 | 4 |
| AD-1446169.1 | 26 | 5 | 27 | 2 | 52 | 1 |
| AD-1446086.1 | 27 | 5 | 34 | 4 | 47 | 7 |
| AD-1446151.1 | 27 | 7 | 28 | 5 | 32 | 7 |
| AD-1285235.1 | 27 | 5 | 23 | 2 | 34 | 7 |
| AD-1446203.1 | 27 | 7 | 28 | 3 | 37 | 3 |
| AD-1446163.1 | 27 | 4 | 28 | 6 | 47 | 3 |
| AD-1446179.1 | 27 | 2 | 27 | 1 | 41 | 5 |
| AD-1446193.1 | 27 | 3 | 41 | 4 | 50 | 9 |
| AD-1285247.1 | 28 | 5 | 33 | 2 | 42 | 11 |
| AD-1446075.1 | 29 | 4 | 41 | 4 | 51 | 4 |
| AD-1446107.1 | 29 | 3 | 30 | 1 | 43 | 10 |
| AD-1446146.1 | 30 | 4 | 31 | 7 | 43 | 4 |
| AD-1285246.1 | 30 | 6 | 29 | 3 | 38 | 5 |
| AD-1285245.2 | 31 | 1 | 31 | 4 | 40 | 6 |
| AD-1446190.1 | 31 | 6 | 35 | 2 | 49 | 10 |
| AD-1446173.1 | 31 | 4 | 39 | 4 | 56 | 4 |
| AD-1285245.1 | 32 | 3 | 30 | 7 | 43 | 6 |
| AD-1446174.1 | 32 | 4 | 38 | 7 | 59 | 8 |
| AD-1446175.1 | 32 | 3 | 38 | 7 | 59 | 5 |
| AD-1446204.1 | 33 | 4 | 28 | 4 | 41 | 7 |
| AD-1446116.1 | 34 | 5 | 27 | 4 | 35 | 4 |
| AD-1446186.1 | 35 | 10 | 40 | 3 | 57 | 8 |
| AD-1446165.1 | 36 | 5 | 39 | 8 | 52 | 5 |
| AD-1446077.1 | 36 | 2 | 37 | 2 | 53 | 8 |
| AD-1446164.1 | 36 | 2 | 28 | 4 | 46 | 9 |
| AD-1446081.1 | 37 | 3 | 39 | 4 | 52 | 4 |
| AD-1446131.1 | 38 | 5 | 42 | 4 | 55 | 5 |
| AD-1446102.1 | 38 | 7 | 45 | 3 | 54 | 11 |
| AD-1446172.1 | 39 | 5 | 49 | 12 | 61 | 9 |
| AD-1446118.1 | 39 | 10 | 37 | 10 | 50 | 11 |
| AD-1446130.1 | 40 | 10 | 42 | 3 | 60 | 4 |
| AD-1446187.1 | 40 | 5 | 43 | 9 | 59 | 2 |
| AD-1446145.1 | 40 | 6 | 50 | 9 | 69 | 14 |
| AD-1446076.1 | 41 | 4 | 38 | 7 | 43 | 5 |
| AD-1446191.1 | 43 | 4 | 36 | 8 | 43 | 8 |
| AD-1446098.1 | 43 | 12 | 45 | 7 | 56 | 7 |
| AD-1446125.1 | 44 | 7 | 50 | 3 | 55 | 7 |
| AD-1446078.1 | 45 | 10 | 42 | 4 | 56 | 6 |
| AD-1446110.1 | 45 | 9 | 45 | 4 | 51 | 7 |
| AD-1446082.1 | 45 | 6 | 40 | 5 | 51 | 7 |
| AD-1446115.1 | 46 | 6 | 50 | 11 | 65 | 5 |
| AD-1446080.1 | 46 | 8 | 52 | 10 | 70 | 14 |
| AD-1446181.1 | 48 | 2 | 52 | 5 | 74 | 11 |
| AD-1446159.1 | 49 | 3 | 43 | 5 | 46 | 4 |
| AD-1446099.1 | 51 | 7 | 60 | 5 | 74 | 11 |
| AD-1446101.1 | 51 | 1 | 56 | 4 | 65 | 13 |
| AD-1446126.1 | 52 | 8 | 49 | 8 | 59 | 11 |
| AD-1446176.1 | 52 | 9 | 49 | 4 | 61 | 4 |
| AD-1446079.1 | 53 | 3 | 41 | 2 | 42 | 3 |
| AD-1446124.1 | 54 | 3 | 61 | 5 | 67 | 4 |
| AD-1446122.1 | 54 | 3 | 72 | 14 | 74 | 20 |
| AD-1446105.1 | 56 | 14 | 61 | 9 | 74 | 9 |
| AD-1446074.1 | 56 | 7 | 75 | 6 | 76 | 8 |
| AD-1446136.1 | 56 | 9 | 63 | 10 | 79 | 6 |
| AD-1446097.1 | 56 | 9 | 67 | 9 | 74 | 16 |
| AD-1446129.1 | 58 | 6 | 54 | 6 | 55 | 3 |
| AD-1446108.1 | 58 | 7 | 56 | 7 | 64 | 9 |
| AD-1446178.1 | 59 | 6 | 57 | 6 | 77 | 4 |
| AD-1446128.1 | 60 | 10 | 54 | 2 | 68 | 5 |
| AD-1446155.1 | 61 | 7 | 54 | 3 | 67 | 9 |
| AD-1446137.1 | 63 | 9 | 62 | 8 | 77 | 8 |
| AD-1446085.1 | 65 | 5 | 60 | 11 | 72 | 6 |
| AD-1446106.1 | 65 | 5 | 66 | 2 | 64 | 13 |
| AD-1446132.1 | 67 | 1 | 77 | 10 | 85 | 8 |
| AD-1446104.1 | 68 | 17 | 57 | 10 | 68 | 12 |
| AD-1446127.1 | 68 | 6 | 54 | 10 | 56 | 3 |
| AD-1446171.1 | 69 | 10 | 78 | 7 | 88 | 9 |
| AD-1446120.1 | 70 | 14 | 79 | 9 | 79 | 20 |
| AD-1446144.1 | 71 | 7 | 81 | 2 | 86 | 2 |
| AD-1446135.1 | 71 | 6 | 69 | 8 | 84 | 15 |
| AD-1446096.1 | 73 | 7 | 68 | 9 | 89 | 8 |
| AD-1446094.1 | 73 | 10 | 78 | 8 | 76 | 19 |
| AD-1446100.1 | 74 | 8 | 68 | 3 | 79 | 4 |
| AD-1446140.1 | 76 | 13 | 81 | 10 | 83 | 11 |
| AD-1446143.1 | 77 | 5 | 73 | 3 | 78 | 12 |
| AD-1446141.1 | 78 | 9 | 82 | 4 | 89 | 16 |
| AD-1446138.1 | 81 | 8 | 89 | 6 | 97 | 16 |
| AD-1446134.1 | 85 | 14 | 81 | 4 | 89 | 15 |
| AD-1446139.1 | 87 | 12 | 91 | 14 | 90 | 19 |
| AD-1446123.1 | 89 | 28 | 100 | 8 | 94 | 14 |
| AD-1446121.1 | 89 | 3 | 89 | 3 | 91 | 8 |
| AD-1446109.1 | 90 | 8 | 93 | 15 | 100 | 10 |
| AD-1446119.1 | 92 | 5 | 85 | 14 | 83 | 5 |
| AD-1446142.1 | 93 | 19 | 89 | 8 | 87 | 10 |
| AD-1446093.1 | NA | 17 | 21 | 3 | 24 | 2 |

TABLE 7a
C9ORF72 RNA target sequences having ≤50% message
remaining for dosing at 0.1 nM as measured in
Table 7.

| Tar- | Tar- | | SEQ |
|---|---|---|---|
| get | get | | ID |
| Start | End | RNA Target Sequence (NG_031977.2) | NO.: |
| 5035 | 5059 | TGCGTCAAACAGCGACAAGTTCCGC | 51 |
| 5058 | 5087 | GCCCACGTAAAAGATGACGCTTGGTGTGTC | 52 |
| 5197 | 5222 | CTTGCTCTCACAGTACTCGCTGAGGG | 53 |
| 5213 | 5270 | TCGCTGAGGGTGAACAAGAAAAGACCTGATAAA | 54 |
| | | GATTAACCAGAAGAAAACAAGGAGG | |
| 5539 | 5565 | TTACTTTCCCTCTCATTTCTCTGACCG | 55 |
| 5545 | 5570 | TCCCTCTCATTTCTCTGACCGAAGCT | 56 |
| 5916 | 5955 | GGAGACGCCTGCACAATTTCAGCCCAAGCTTCTA | 57 |
| | | GAGAGT | |
| 5935 | 5968 | CAGCCCAAGCTTCTAGAGAGTGGTGATGACTTGC | 58 |
| 5948 | 5976 | TAGAGAGTGGTGATGACTTGCATATGAGG | 59 |
| 6007 | 6030 | CTGTGGGACATGACCTGGTTGCTT | 60 |
| 6012 | 6039 | GGACATGACCTGGTTGCTTCACAGCTCC | 61 |
| 6020 | 6070 | CCTGGTTGCTTCACAGCTCCGAGATGACACAGAC | 62 |
| | | TTGCTTAAAGGAAGTGA | |

TABLE 7b
C9ORF72 RNA target sequences having ≤40% message
remaining for dosing at 0.1 nM as measured in
Table 7.

| Tar- | Tar- | | SEQ |
|---|---|---|---|
| get | get | | ID |
| Start | End | RNA Target Sequence (NG_031977.2) | NO.: |
| 5035 | 5059 | TGCGTCAAACAGCGACAAGTTCCGC | 63 |
| 5059 | 5084 | CCCACGTAAAAGATGACGCTTGGTGT | 64 |
| 5064 | 5087 | GTAAAAGATGACGCTTGGTGTGTC | 65 |
| 5197 | 5222 | CTTGCTCTCACAGTACTCGCTGAGGG | 66 |
| 5213 | 5270 | TCGCTGAGGGTGAACAAGAAAAGACCTGATAAA | 67 |
| | | GATTAACCAGAAGAAAACAAGGAGG | |
| 5539 | 5565 | TTACTTTCCCTCTCATTTCTCTGACCG | 68 |
| 5545 | 5569 | TCCCTCTCATTTCTCTGACCGAAGC | 69 |
| 5921 | 5955 | CGCCTGCACAATTTCAGCCCAAGCTTCTAGAGAG | 70 |
| | | T | |
| 5939 | 5963 | CCAAGCTTCTAGAGAGTGGTGATGA | 71 |
| 5948 | 5973 | TAGAGAGTGGTGATGACTTGCATATG | 72 |
| 6012 | 6039 | GGACATGACCTGGTTGCTTCACAGCTCC | 73 |
| 6036 | 6059 | CTCCGAGATGACACAGACTTGCTT | 74 |
| 6040 | 6066 | GAGATGACACAGACTTGCTTAAAGGAA | 75 |

TABLE 7c
C9ORF72 RNA target sequences having ≤30% message
remaining for dosing at 0.1 nM as measured in
Table 7.

| Tar- | Tar- | | SEQ |
|---|---|---|---|
| get | get | | ID |
| Start | End | RNA Target Sequence (NG_031977.2) | NO.: |
| 5036 | 5059 | GCGTCAAACAGCGACAAGTTCCGC | 76 |
| 5064 | 5087 | GTAAAAGATGACGCTTGGTGTGTC | 77 |
| 5223 | 5270 | TGAACAAGAAAAGACCTGATAAAGATTAACCAG | 78 |
| | | AAGAAAACAAGGAGG | |
| 5539 | 5563 | TTACTTTCCCTCTCATTTCTCTGAC | 79 |
| 5939 | 5962 | CCAAGCTTCTAGAGAGTGGTGATG | 80 |
| 6016 | 6039 | ATGACCTGGTTGCTTCACAGCTCC | 81 |
| 6036 | 6059 | CTCCGAGATGACACAGACTTGCTT | 82 |
| 6040 | 6065 | GAGATGACACAGACTTGCTTAAAGGA | 83 |

TABLE 7d
C9ORF72 RNA target sequences having ≤25% message
remaining for dosing at 0.1 nM as measured in
Table 7.

| Tar- | Tar- | | SEQ |
|---|---|---|---|
| get | get | | ID |
| Start | End | RNA Target Sequence (NG_031977.2) | NO.: |
| 5036 | 5059 | GCGTCAAACAGCGACAAGTTCCGC | 84 |
| 5223 | 5270 | TGAACAAGAAAAGACCTGATAAAGATTAACCAG | 85 |
| | | AAGAAAACAAGGAGG | |
| 5539 | 5562 | TTACTTTCCCTCTCATTTCTCTGA | 86 |
| 6016 | 6039 | ATGACCTGGTTGCTTCACAGCTCC | 87 |
| 6036 | 6059 | CTCCGAGATGACACAGACTTGCTT | 88 |
| 6040 | 6065 | GAGATGACACAGACTTGCTTAAAGGA | 89 |

TABLE 7e
C9ORF72 RNA target sequences having ≤20% message
remaining for dosing at 0.1 nM as measured in
Table 7.

| | | | SEQ |
|---|---|---|---|
| Target | Target | RNA Target Sequence | ID |
| Start | End | (NG_031977.2) | NO.: |
| 5229 | 5252 | AGAAAAGACCTGATAAAGATTAAC | 90 |
| 5233 | 5256 | AAGACCTGATAAAGATTAACCAGA | 91 |
| 5539 | 5562 | TTACTTTCCCTCTCATTTCTCTGA | 92 |
| 6036 | 6059 | CTCCGAGATGACACAGACTTGCTT | 93 |

### Example 3. In Vivo Evaluation in Transgenic Mice

This Example describes methods for the evaluation of C9orf72 RNAi agents in an allelic series of genetically modified mice (U.S. Pat. No. 10,781,453, WO/2018/064600, US2020/0196581 and WO/2020/131632, the entire contents of each of the foregoing application are herein incorporated by reference). The allelic series comprises a set of mouse ES cells with in which a portion of the mouse C9orf72 gene that includes exons 1A and 1B and adjacent intron sequences is precisely replaced with the homologous fragment from the human C9orf72 gene carrying varying lengths of GGGGCC hexanucleotide repeat expansion (SEQ ID NO: 100) from the normal range of 3 to 30 repeats to over 500 repeats. ES cells of the allelic series are used to derive mice by standard methods, such as the VelociMouse method of 8-cell embryo injection (Poueymirou et al., 2007).

dsRNA agents designed and assayed in Example 5 are assessed for their ability to reduce the level of sense- or antisense GGGGCC repeat-containing (SEQ ID NO: 100) or intron-containing RNA or spliced RNA from exons 1A and 1B or total C9orf72 mRNA by, for example, in fluorescence situ hybridization (FISH) for RNA foci with strand-specific probes (Exiqon, Inc.), reverse transcription-coupled quantitative PCR (R-qPCR), or by hybridization to strand-specific probes with, for example, Nanostring®, QantiGene®, or Lucerna® assay technologies in mice of the allelic series.

Briefly, heterozygous or homozygous mice with up to or greater than 500 GGGGCC repeats are administered by intracerebroventrical, intrathecal, or subcutaneous injection a single dose of the dsRNA agents of interest, including duplexes AD-463858, AD-463860, AD-463862. AD-463863, AD-463869, AD-463871. AD-463872, AD-463873, AD-463877, or a placebo. Two to 10 weeks post-administration, animals are sacrificed, blood and tissue samples, including cerebral cortex, spinal cord, liver, spleen, and cervical lymph nodes, are collected, and RNA is purified from the tissue samples. Repeat- or intron-containing or normal RNA produced from the genetically modified C9orf72 gene is assayed by RNA FISH, RT-qPCR, or strand-specific detection methods. Results from mice carrying long GGGGCC repeat expansions up to or greater than 500 repeats are compared to control mice carrying normal repeat lengths of between 3 and 30 repeats.

In addition to RNA, protein is extracted from and tissues of the mice and assayed for the presence of pathogenic dipeptide repeat proteins, including poly(GlyPro), poly(GlyAla), poly(GlyArg), poly(ProAla), and poly(ProArg), produced by repeat-associated non-AUG and canonical translation of C9orf72 sense and antisense GGGGCC repeat (SEQ ID NO: 100) containing transcripts. Dipeptide repeat proteins and normal C9orf72 proteins are assayed in mouse tissues with available antibodies against the individual dipeptide repeat proteins by immunohistochemistry, western blotting, enzyme-linked immunosorbent assays, and MesoScale Discovery® assays. Results from cells and mice carrying long GGGGCC repeat expansions up to or greater than 500 repeats are compared to cells carrying normal repeat lengths of between 3 and 30 repeats.

The results demonstrate that administration of the dsRNA agents to mice of the allelic series inhibits the production of sense repeat- and intron-containing and antisense repeat-containing C9orf72 transcripts but has no impact on the level of C9orf72 total and exon 1B-containing mRNA levels. The results also demonstrate that administration of the dsRNA agents inhibits the production of dipeptide repeat proteins derived from the sense and antisense repeat-containing C9orf72 transcripts but has no impact on the level of normal C9orf72 proteins. The results demonstrate that administration of the dsRNA agents reduces the level of sense- and antisense repeat-containing RNA throughout the central nervous system, including the brain, brainstem, and spinal cord. The results demonstrate that maximal reduction of dipeptide repeat proteins produced by mice of the GGGGCC repeat expansion (SEQ ID NO: 100) allelic series is obtained by dsRNA agents that target both the C9orf72 sense and antisense GGGGCC repeat-containing transcripts (SEQ ID NO: 100).

| | | SEQ | | SEQ |
|---|---|---|---|---|
| Duplex | | ID | | ID |
| Name | Sense transSeq | NO: | Antisense transSeq | NO: |
| AD-463858 | ACAAGAAAAGACCUGAUAAAU | 1294 | AUUUAUCAGGUCUUUUCUUGUUC | 1302 |
| AD-463860 | AGAAAAGACCUGAUAAAGAUU | 1295 | AAUCUUUAUCAGGUCUUUUCUUG | 1421 |
| AD-463862 | AAAAGACCUGAUAAAGAUUAA | 599 | UUAAUCUUUAUCAGGUCUUUUCU | 1419 |
| AD-463863 | AAAGACCUGAUAAAGAUUAAU | 1296 | AUUAAUCUUUAUCAGGUCUUUUC | 1303 |
| AD-463869 | CUGAUAAAGAUUAACCAGAAU | 1297 | AUUCUGGUUAAUCUUUAUCAGGU | 1304 |
| AD-463871 | GAUAAAGAUUAACCAGAAGAA | 1298 | UUCUUCUGGUUAAUCUUUAUCAG | 1417 |
| AD-463872 | AUAAAGAUUAACCAGAAGAAA | 1299 | UUUCUUCUGGUUAAUCUUUAUCA | 1416 |
| AD-463873 | AAAGAUUAACCAGAAGAAAAU | 1300 | AUUUUCUUCUGGUUAAUCUUUAU | 1305 |
| AD-463877 | UAACCAGAAGAAAACAAGGAU | 1301 | AUCCUUGUUUUCUUCUGGUUAAU | 1306 |
| | | SEQ | | SEQ |
| Duplex | | ID | | ID |
| Name | Sense oligoSeq | NO: | Antisense oligoSeq | NO: |
| AD-463858 | ascsaagaAfaAfGfAfccugauaaauL96 | 1307 | asUfsuuaUfcAfGfgucuUfuUfcuugususc | 1316 |
| AD-463860 | asgsaaaaGfaCfCfUfgauaaagauuL96 | 1308 | asAfsucuUfuAfUfcaggUfcUfuuucususg | 1317 |
| AD-463862 | asasaagaCfcUfGfAfuaaagauuaaL96 | 1309 | usUfsaauCfuUfUfaucaGfgUfcuuuuscsu | 1318 |
| AD-463863 | asasagacCfuGfAfUfaaagauuaauL96 | 1310 | asUfsuaaUfcUfUfuaucAfgGfucuuususc | 1319 |
| AD-463869 | csusgauaAfaGfAfUfuaaccagaauL96 | 1311 | asUfsucuGfgUfUfaaucUfuUfaucagsgsu | 1320 |
| AD-463871 | gsasuaaaGfaUfUfAfaccagaagaaL96 | 1312 | usUfscuuCfuGfGfuuaaUfcUfuuaucsasg | 1321 |
| AD-463872 | asusaaagAfuUfAfAfccagaagaaaL96 | 1313 | usUfsucuUfcUfGfguuaAfuCfuuuauscsa | 1322 |
| AD-463873 | asasagauUfaAfCfCfagaagaaaauL96 | 1314 | asUfsuuuCfuUfCfugguUfaAfucuuusasu | 1323 |
| AD-463877 | usasaccaGfaAfGfAfaaacaaggauL96 | 1315 | asUfsccuUfgUfUfuucuUfcUfgguuasasu | 1324 |

### Example 4. Additional Agents Targeting C9orf72

Additional agents targeting C9orf72 were designed and synthesized as described above. The unmodified nucleotide sequences of these agents are provided in Table 8 and the modified nucleotide sequences of these agents are provided in Table 9.

TABLE 8
Unmodified Sense and Antisense Strand Sequences of dsRNA Agents Targeting C90rf72

| | | SEQ | | | SEQ | |
|---|---|---|---|---|---|---|
| Duplex | Sense Sequence | ID | Range in | Antisense Sequence | ID | Range in |
| Name | 5′ to 3′ | NO: | NM_001256054 | 5′ to 3′ | NO: | NM_001256054 |
| AD-1285248 | CAUAUGGACUAUCAAUUAUAA | 1325 | 1092-1112 | UUAUAATUGAUAGUCCAUAUGUG | 1341 | 526-548 |
| AD-1285249 | UGUUGCCAAGACAGAGAUUGA | 1326 | 375-395 | UCAAUCTCUGUCUUGGCAACAGC | 1342 | 233-255 |
| AD-1285250 | CAAGACAGAGAUUGCUUUAAA | 1327 | 2015-2035 | UUUAAAGCAAUCUCUGUCUUGGC | 1343 | 239-261 |
| AD-1285251 | UAAAUGGAGAAAUCCUUCGAA | 1328 | 1092-1112 | UUCGAAGGAUUUCUCCAUUUAGA | 1344 | 400-422 |
| AD-1285252 | UGUGUGUUGAUAGAUUAACAA | 1329 | 594-614 | UUGUUAAUCUAUCAACACACACU | 1345 | 589-611 |
| AD-1285253 | CAGAACUUAGUUUCUACCUCA | 1330 | 3227-3247 | UGAGGUAGAAACUAAGUUCUGUC | 1346 | 556-578 |
| AD-1285254 | ACAGAACUUAGUUUCUACCUA | 1331 | 3228-3248 | UAGGUAGAAACUAAGUUCUGUCU | 1347 | 555-577 |
| AD-1285255 | UGGACUAUCAAUUAUACUUCA | 1332 | 928-948 | UGAAGUAUAAUUGAUAGUCCAUA | 1348 | 530-552 |
| AD-1285256 | AGUGAUGUCGACUCUUUGCCA | 1333 | 760-780 | UGGCAAAGAGUCGACAUCACUGC | 1349 | 197-219 |
| AD-1285257 | AAGACAGAGAUUGCUUUAAGA | 1334 | 1539-1559 | UCUUAAAGCAAUCUCUGUCUUGG | 1350 | 240-262 |
| AD-1285258 | AAUAUUCUUGGUCCUAGAGUA | 1335 | 616-636 | UACUCUAGGACCAAGAAUAUUGU | 1351 | 303-325 |
| AD-1285259 | UGAUACAGUACUCAAUGAUGA | 1336 | 3089-3109 | UCAUCATUGAGUACUGUAUCAGC | 1352 | 806-828 |
| AD-1285260 | UAGCUGAUACAGUACUCAAUA | 1337 | 2131-2151 | UAUUGAGUACUGUAUCAGCUAUA | 1353 | 802-824 |
| AD-1285261 | CUGUCAUGAAGGCUUUCUUCA | 1338 | 373-393 | UGAAGAAAGCCUUCAUGACAGCU | 1354 | 842-864 |
| AD-1285262 | ACAUAUUUAUAAUCAGCGUAA | 1339 | 1006-1026 | UUACGCTGAUUAUAAAUAUGUUC | 1355 | 1169-1191 |
| AD-1285263 | GUCUUACACAGAGACACUCUA | 1340 | 1581-1601 | UAGAGUGUCUCUGUGUAAGACAU | 1356 | 1308-1330 |

TABLE 9
Modified Sense and Antisense Strand Sequences of dsRNA Agents Targeting C90rf72

| | | SEQ | | SEQ |
|---|---|---|---|---|
| Duplex | | ID | | ID |
| Name | Sense Sequence 5′ to 3′ | NO: | Antisense Sequence 5′ to 3′ | NO: |
| AD- | csasuau(Ghd)GfaCfUfAfucaauuausasa | 1357 | VPusUfsauaa(Tgn)ugauagUfcCfauaugsusg | 1373 |
| 1285248 | | | | |
| AD- | usgsuug(Chd)CfaAfGfAfcagagauusgsa | 1358 | VPusCfsaauc(Tgn)cugucuUfgGfcaacasgsc | 1374 |
| 1285249 | | | | |
| AD- | csasaga(Chd)AfgAfGfAfuugcuuuasasa | 1359 | VPusUfsuaaa(Ggn)caaucuCfuGfucuugsgsc | 1375 |
| 1285250 | | | | |
| AD- | usasaau(Ghd)GfaGfAfAfauccuucgsasa | 1360 | VPusUfscgaa(Ggn)gauuucUfcCfauuuasgsa | 1376 |
| 1285251 | | | | |
| AD- | usgsugu(Ghd)UfuGfAfUfagauuaacsasa | 1361 | VPusUfsguua(Agn)ucuaucAfaCfacacascsu | 1377 |
| 1285252 | | | | |
| AD- | csasgaa(Chd)UfuAfGfUfuucuaccuscsa | 1362 | VPusGfsaggu(Agn)gaaacuAfaGfuucugsus | 1378 |
| 1285253 | | | c | |
| AD- | ascsaga(Ahd)CfuUfAfGfuuucuaccsusa | 1363 | VPusAfsggua(Ggn)aaacuaAfgUfucuguscs | 1379 |
| 1285254 | | | u | |
| AD- | usgsgac(Uhd)AfuCfAfAfuuauacuuscsa | 1364 | VPusGfsaagu(Agn)uaauugAfuAfguccasus | 1380 |
| 1285255 | | | a | |
| AD- | asgsuga(Uhd)GfuCfGfAfcucuuugcscsa | 1365 | VPusGfsgcaa(Agn)gagucgAfcAfucacusgsc | 1381 |
| 1285256 | | | | |
| AD- | asasgac(Ahd)GfaGfAfUfugcuuuaasgsa | 1366 | VPusCfsuuaa(Agn)gcaaucUfcUfgucuusgsg | 1382 |
| 1285257 | | | | |
| AD- | asasuau(Uhd)CfuUfGfGfuccuagagsusa | 1367 | VPusAfscucu(Agn)ggaccaAfgAfauauusgs | 1383 |
| 1285258 | | | u | |
| AD- | usgsaua(Chd)AfgUfAfCfucaaugausgsa | 1368 | VPusCfsauca(Tgn)ugaguaCfuGfuaucasgsc | 1384 |
| 1285259 | | | | |
| AD- | usasgcu(Ghd)AfuAfCfAfguacucaasusa | 1369 | VPusAfsuuga(Ggn)uacuguAfuCfagcuasus | 1385 |
| 1285260 | | | a | |
| AD- | csusguc(Ahd)UfgAfAfGfgcuuucuuscs | 1370 | VPusGfsaaga(Agn)agccuuCfaUfgacagscsu | 1386 |
| 1285261 | a | | | |
| AD- | ascsaua(Uhd) UfuAfUfAfaucagcgusasa | 1371 | VPusUfsacgc(Tgn)gauuauAfaAfuaugususc | 1387 |
| 1285262 | | | | |
| AD- | gsuscuu(Ahd)CfaCfAfGfagacacucsusa | 1372 | VPusAfsgagu(Ggn)ucucugUfgUfaagacsas | 1388 |
| 1285263 | | | u | |
| | | | | SEQ |
| | | | | ID |
| | | | mRNA Target Sequence | NO: |
| | | | CACAUAUGGACUAUCAA | 1389 |
| | | | UUAUAC | |
| | | | GCUGUUGCCAAGACAGA | 1390 |
| | | | GAUUGC | |
| | | | GCCAAGACAGAGAUUGC | 1391 |
| | | | UUUAAG | |
| | | | UCUAAAUGGAGAAAUCC | 1392 |
| | | | UUCGAA | |
| | | | AGUGUGUGUUGAUAGA | 1393 |
| | | | U | |
| | | | UAACAC | |
| | | | GACAGAACUUAGUUUCU | 1394 |
| | | | ACCUCC | |
| | | | AGACAGAACUUAGUUUC | 1395 |
| | | | UACCUC | |
| | | | UAUGGACUAUCAAUUAU | 1396 |
| | | | ACUUCC | |
| | | | GCAGUGAUGUCGACUCU | 1397 |
| | | | UUGCCC | |
| | | | CCAAGACAGAGAUUGCU | 1398 |
| | | | UUAAGU | |
| | | | ACAAUAUUCUUGGUCCU | 1399 |
| | | | AGAGUA | |
| | | | GCUGAUACAGUACUCAA | 1400 |
| | | | UGAUGA | |
| | | | UAUAGCUGAUACAGUAC | 1401 |
| | | | UCAAUG | |
| | | | AGCUGUCAUGAAGGCUU | 1402 |
| | | | UCUUCU | |
| | | | GAACAUAUUUAUAAUCA | 1403 |
| | | | GCGUAG | |
| | | | AUGUCUUACACAGAGA | 1404 |
| | | | CACUCUA | |

### Example 5. In Vitro Evaluation of Compostions Comprising Two or More dsRNA Agents Targeting C9orf72

Sense and antisense repeat expansion RNA detected as cytoplasmic and nuclear foci by fluorescence in situ hybridization (FISH) may sequester RNA binding proteins, leading to cellular toxicity. In addition, dipeptide repeat (DPR) proteins are proposed to be produced from the G₄C₂ repeat expansion (SEQ ID NO: 100) sense and antisense RNA by a non-canonical process that has been termed repeat associated non-AUG (RAN) translation, and there is strong evidence that DPR proteins are cytotoxic. DPR proteins which can be translated from all sense and antisense reading frames. Sense DPR proteins include glycine-alanine, glycine-arginine, and glycine-proline DPR proteins. Antisense DPR proteins include proline-arginine, proline-alanine, and glycine-proline. Because G+C2 (SEQ ID NO: 100) repeat-containing RNAs, either on their own or as templates for dipeptide repeat protein translation, appear to be pathogenic, a general therapeutic strategy is to either inhibit their synthesis or promote their destruction. In the example below, it is demonstrated that siRNAs that target both C9orf72 sense and antisense RNAs are required to achieve maximum knockdown of dipeptide repeat proteins in humanized C9orf72 models.

RNA interference was explored as a modality for the destruction of C9orf72 G&C2 (SEQ ID NO: 100) repeat-containing RNA. Because the G+C2 repeat (SEQ ID NO: 100) itself and the GC-rich sequence immediately 3′-adjacent are not compatible with specific siRNA design and targeting sequences in exon 1B (E1B) and its adjacent intron could interfere with C9orf72 mRNA, siRNA designs were focused on sense and antisense RNAs carrying sequences derived from the region of the human C9orf72 gene between E1A and the start of the repeat expansion. Four siRNAs were tested, two targeting sense RNA and two targeting antisense transcripts, in mouse ES cells with the 300X repeat expansion allele. The unmodified and modified nucleotide sequences of the agents used are provided in Tables 10A-10D, below.

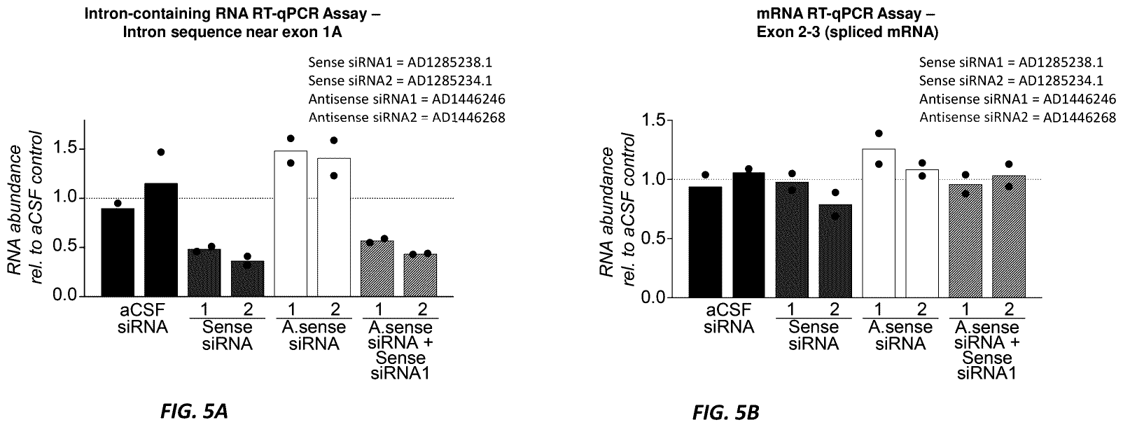

The siRNAs targeting sense RNA produced a 50-60% reduction of intron-containing transcripts (FIG. 5A) as determined by a RT-qPCR assay for intron sequence near E1A. The two antisense-targeting siRNAs produced a 40-50% increase in signal with the same assay (FIG. 5A). Combining one of the sense-targeting siRNAs with either of the two antisense-targeting siRNAs did not cause a further knockdown of intron-containing RNA than that produced by the sense-targeting siRNA alone (FIG. 5A). Neither the sense-nor antisense-targeting siRNAs had an appreciable effect on the C9orf72 mRNA (FIG. 5B).

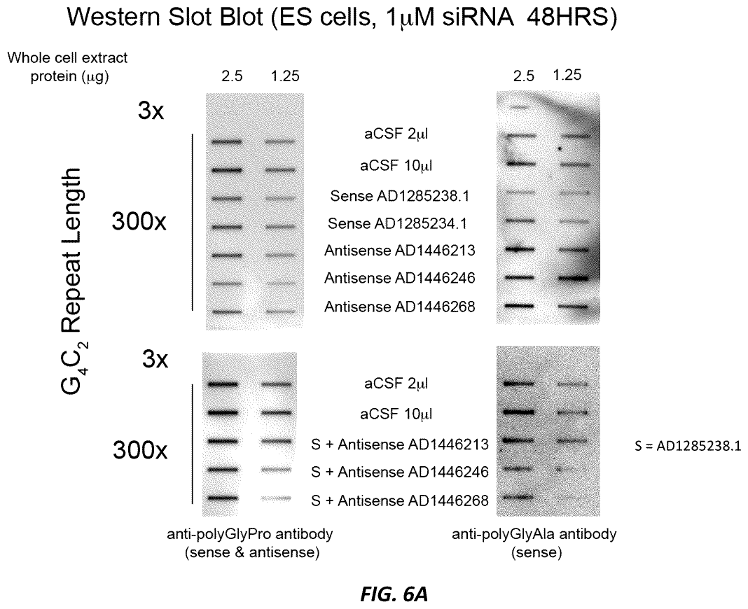

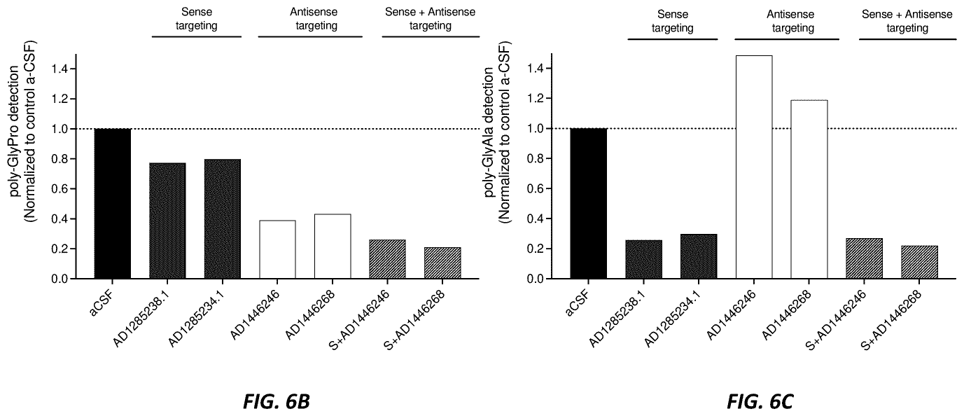

The effect of the siRNAs on DPR protein synthesis by western slot blotting was also assayed (FIG. 6A). Quantitative analysis of these assays revealed that relative to the vehicle control both sense-targeting siRNAs reduced poly(GlyAla) by approximately 75% (FIG. 6B), while the antisense-targeting siRNAs actually caused a slight increase in poly(Gly Ala) (FIG. 6B), consistent with the moderate increase in intron-containing RNA produced by these siRNAs (FIG. 5A). The combination of sense- and antisense-targeting siRNAs did not enhance the inhibition of poly(GlyAla) achieved by the sense-targeting siRNA alone. The sense-targeting siRNAs inhibited poly(GlyPro) synthesis by approximately 20%, while the antisense-targeting siRNAs produced a stronger 60-70% knockdown (FIG. 6C). Combining the sense- and antisense-targeting siRNAs further reduced poly(GlyPro) synthesis, achieving an approximately 80% knockdown (FIG. 6C). These results support the expectation that a sense RNA serves as the template for translation of poly(GlyAla), while poly(GlyPro) is synthesized from both sense and antisense RNA templates. The stronger inhibition of poly(GlyPro) synthesis achieved with the antisense-targeting siRNAs indicates that the majority of this DPR protein is produced from antisense transcripts in the 300X model. With poly(GlyAla) and poly(GlyPro) as the surrogates for sense (poly(GlyAla), poly(GlyPro), and poly(GlyArg)) and antisense (poly(GlyPro), poly(AlaPro), and poly(ProArg)) RNA DPR synthesis, respectively, the results demonstrate that therapeutic RNAi for C9orf72 ALS may require siRNAs that target both the sense and antisense transcripts to achieve maximal inhibition of DPR protein synthesis.

TABLE 10A
Unmodified Nucleotide Sequences of Antisense-Targeting RNAi Agents

| | | SEQ | | SEQ |
|---|---|---|---|---|
| Duplex | | ID | | ID |
| Name | Sense | NO: | Antisense | NO: |
| AD- | GCUUCGGUCAGAGAAAUGAGA | 116 | UCUCAUUUCUCUGACCGAAGCUG | 1410 |
| 1446213 | | | | |
| AD- | UUCCCUCCUUGUUUUCUUCUA | 149 | UAGAAGAAAACAAGGAGGGAAAC | 239 |
| 1446246 | | | | |
| AD- | CUUUAUCAGGUCUUUUCUUGA | 171 | UCAAGAAAAGACCUGAUAAAGAU | 261 |
| 1446268 | | | | |

TABLE 10B
Unmodified Nucleotide Sequences of Sense-Targeting RNAi Agents

| | | SEQ | Antisense | SEQ |
|---|---|---|---|---|
| Duplex | | ID | | ID |
| Name | Sense | NO: | | NO: |
| AD- | AACAAGAAAAGACCUGAUAAA | 595 | UUUAUCAGGUCUUUUCUUGUUCA | 1422 |
| 1285238 | | | | |
| AD- | AGAAAAGACCUGAUAAAGAUA | 597 | UAUCUUUAUCAGGUCUUUUCUUG | 744 |
| 1285234 | | | | |

TABLE 10C
Modified Nucleotide Sequences of Antisense-Targeting RNAi Agents

| | | SEQ | | SEQ |
|---|---|---|---|---|
| | | ID | | ID |
| Duplex Name | Sense | NO: | Antisense | NO: |
| AD- | gscsuuc(Ghd)GfuCfAfGfagaaaugasgsa | 296 | VPusCfsucaUfuUfCfucugAfcCfgaagcsusg | 386 |
| 1446213.1 | | | | |
| AD- | ususccc(Uhd)CfcUfUfGfuuuucuucsusa | 329 | VPusAfsgaaGfaAfAfacaaGfgAfgggaasasc | 419 |
| 1446246.1 | | | | |
| AD- | csusuua(Uhd)CfaGfGfUfcuuuucuusgsa | 351 | VPusCfsaagAfaAfAfgaccUfgAfuaaagsasu | 441 |
| 1446268.1 | | | | |

TABLE 10D
Modified Nucleotide Sequences of Sense-Targeting RNAi Agents

| | | SEQ | | SEQ |
|---|---|---|---|---|
| Duplex | | ID | | ID |
| Name | Sense | NO: | Antisense | NO: |
| AD- | asascaa(Ghd)AfaAfAfGfaccugauasasa | 884 | VPusUfsuauCfaGfGfucuuUfuCfuuguuscsa | 1033 |
| 1285238.1 | | | | |
| AD- | asgsaaa(Ahd)GfaCfCfUfgauaaagasusa | 886 | VPusAfsucuUfuAfUfcaggUfcUfuuucususg | 1035 |
| 1285234.1 | | | | |

### Example 6. In vivo screening of dsRNA Duplexes in Mice

Duplexes targeting the antisense strand of intron 1A of C9orf72 were evaluated in vivo.

Table 11 provides the unmodified sense and antisense strand nucleotide sequences of the agents targeting the antisense strand of C9orf72 intron 1A and Table 12 provides the modified sense and antisense strand nucleotide sequences of the agents targeting the antisense strand of C9orf72 intron 1A used in this study.

At pre-dose day-14 wild-type mice (C57BL/6) were transduced with either 2×10¹⁰ or 2×10¹¹ viral particles of an adeno-associated virus 8 (AAV8) vector including a region between exon 1A and the repeat expansion of human C9orf72, which includes a portion of intron 1A by intravenous administration. The antisense vector sequence is provided in SEQ ID NO: 94.

At day 0, groups of three mice were intrathecally administered a single 3 mg/kg dose of the agents of interest, a single 3 mg/kg or 10 mg/kg dose of a dsRNA agent targeting a gene other than C9orf72 as a positive control, or PBS control. At day 14 post-dose animals were sacrificed, brain samples were collected and snap-frozen in liquid nitrogen. Brain RNA was extracted and analyzed by the RT-QPCR method.

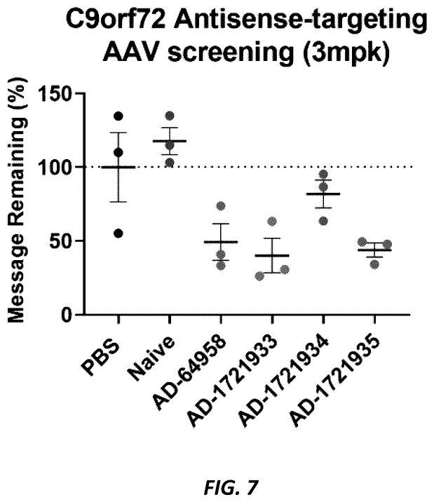

Intronic probes were used to detect the region within the intron. Antisense intron levels of human C9orf72 were compared to a housekeeping gene, GAPDH. The values were then normalized to the average of PBS vehicle control group. The data were expressed as percent of baseline value, and presented as mean plus standard deviation. The results, listed in Table 13 and shown in FIG. 7, demonstrate that the exemplary duplex agents tested that target the antisense strand of intron 1A of human C9orf72 potently reduce the level of the human C9orf92 antisense RNA in vivo.

TABLE 11
Unmodified Nucleotide Sequences of Intron 1A Antisense-Targeting RNAi Agents

| | | SEQ | | SEQ |
|---|---|---|---|---|
| | | ID | | ID |
| Duplex Name | Sense Strand 5′ to 3′ | NO: | Antisense Strand 5′ to 3′ | NO: |
| AD-1721933.1 | CUUUAUCAGGUCUUUUCUUGA | 171 | UCAAGAAAAGACCUGAUAAAGAU | 261 |
| AD-1721934.1 | UUCUGGUUAAUCUUUAUCAGA | 162 | UCUGAUAAAGAUUAACCAGAAGA | 252 |
| AD-1721935.1 | CUUGUUUUCUUCUGGUUAAUA | 155 | UAUUAACCAGAAGAAAACAAGGA | 245 |

TABLE 12
Modified Nucleotide Sequences of Intron 1A Antisense-Targeting RNAi Agents

| | | SEQ | | SEQ |
|---|---|---|---|---|
| Duplex | | ID | | ID |
| Name | Sense Strand 5′ to 3′ | NO: | Antisense Strand 5′ to 3′ | NO: |
| AD- | csusuua(Uhd)CfaGfGfUfcuuuucuugaL96 | 1405 | VPusCfsaagAfaAfAfgaccUfgAfuaaagsasu | 441 |
| 1721933.1 | | | | |
| AD- | ususcug(Ghd)UfuAfAfUfcuuuaucagaL96 | 1406 | VPusCfsugaUfaAfAfgauuAfaCfcagaasgsa | 432 |
| 1721934.1 | | | | |
| AD- | csusugu(Uhd)UfuCfUfUfcugguuaauaL96 | 1407 | VPusAfsuuaAfcCfAfgaagAfaAfacaagsgsa | 425 |
| 1721935.1 | | | | |

TABLE 13

| Group | Animal | | AAV | | | | | normalized | grp |
|---|---|---|---|---|---|---|---|---|---|
| # | # | Treatment | Titer | Dose | avg/mouse | grp avg | stdev | to 100 | average |
| 1 | 1 | PBS | 2.00E+10 | n/a | 145.4583443 | 110.0603 | 42.5617662 | 132.1624 | 100 |
| | 2 | | | | 121.8865945 | | | 110.7452 | |
| | 3 | | | | 62.8360996 | | | 57.09241 | |
| 2 | 4 | Naive | | n/a | 108.7414693 | 82.25354 | 55.868879 | 98.80168 | 74.73495 |
| | 5 | | | | 18.06722083 | | | 16.41574 | |
| | 6 | | | | 119.9519394 | | | 108.9874 | |
| 3 | 7 | AD-64958 | | 3 | 44.82748779 | 49.17021 | 3.84795464 | 40.72992 | 44.67568 |
| | 8 | (control) | | | 52.15540902 | | | 47.38801 | |
| | 9 | | | | 50.52772992 | | | 45.90911 | |
| 4 | 10 | AD-64958 | | 10 | 57.90374506 | 51.88677 | 19.5410098 | 52.61091 | 47.14393 |
| | 11 | (control) | | | 67.7117117 | | | 61.52235 | |
| | 12 | | | | 30.04485251 | | | 27.29853 | |
| 5 | 13 | AD-1721933.1 | | 3 | 31.8631398 | 68.36251 | 32.7786689 | 28.95061 | 62.11366 |
| | 14 | | | | 95.2890682 | | | 86.57893 | |
| | 15 | | | | 77.935321 | | | 70.81144 | |
| 6 | 16 | AD-1721934.1 | | 3 | 82.4652133 | 82.03793 | 10.6144606 | 74.92727 | 74.53904 |
| | 17 | | | | 71.21627551 | | | 64.70657 | |
| | 18 | | | | 92.43229251 | | | 83.98328 | |
| 7 | 19 | AD-1721935.1 | | 3 | 45.68231369 | 75.5266 | 29.5045999 | 41.50661 | 68.6229 |
| | 20 | | | | 76.21814549 | | | 69.25123 | |
| | 21 | | | | 104.6793558 | | | 95.11087 | |
| 8 | 22 | PBS | 2.00E+11 | n/a | 117.7590945 | 106.9963 | 43.5190329 | 110.059 | 100 |
| | 23 | | | | 59.10574588 | | | 55.24093 | |
| | 24 | | | | 144.1240461 | | | 134.7 | |
| 9 | 25 | Naive | | n/a | 122.9141465 | 125.8956 | 17.1442589 | 114.877 | 117.6635 |
| | 26 | | | | 110.4376191 | | | 103.2163 | |
| | 27 | | | | 144.3350411 | | | 134.8972 | |
| 10 | 28 | AD-64958 | | 3 | 78.98649992 | 52.80346 | 23.046316 | 73.82171 | 49.35074 |
| | 29 | (control) | | | 35.59259337 | | | 33.26526 | |
| | 30 | | | | 43.83128886 | | | 40.96524 | |
| 11 | 31 | AD-64958 | | 10 | 103.7821236 | 60.751 | 37.3784316 | 96.996 | 56.77861 |
| | 32 | (control) | | | 36.33902969 | | | 33.96289 | |
| | 33 | | | | 42.13185889 | | | 39.37693 | |
| 12 | 34 | AD-1721933.1 | | | 28.02191177 | 42.90744 | 21.6991653 | 26.18961 | 40.1018 |
| | 35 | | | 3 | 67.80499251 | | | 63.37135 | |
| | 36 | | | | 32.89542331 | | | 30.74445 | |
| 13 | 37 | AD-1721934.1 | | | 68.07513475 | 87.5624 | 17.4973685 | 63.62382 | 81.83686 |
| | 38 | | | 3 | 92.68622676 | | | 86.62564 | |
| | 39 | | | | 101.9258501 | | | 95.2611 | |
| 14 | 40 | AD-1721935.1 | | | 51.21572748 | 46.91163 | 8.93757275 | 47.86682 | 43.84416 |
| | 41 | | | 3 | 36.636385 | | | 34.2408 | |
| | 42 | | | | 52.88276877 | | | 49.42486 | |

| Duplex | | | SEQ ID | | SEQ ID |
|---|---|---|---|---|---|
| ID | Strand | Modified Sequence | NO: | Unmodified Sequence | NO: |
| AD- | sense | asascaguGfuUfCfUfugcucuauaaL96 | 1293 | AACAGUGUUCUUGCUCUAUAA | 103 |
| 64958 | antisense | usUfsauaGfagcaagaAfcAfcuguususu | 102 | UUAUAGAGCAAGAACACUGUUUU | 108 |
| (control) | | | | | |

### Example 7. Mapping of the C9orf72 antisense RNA transcription start site

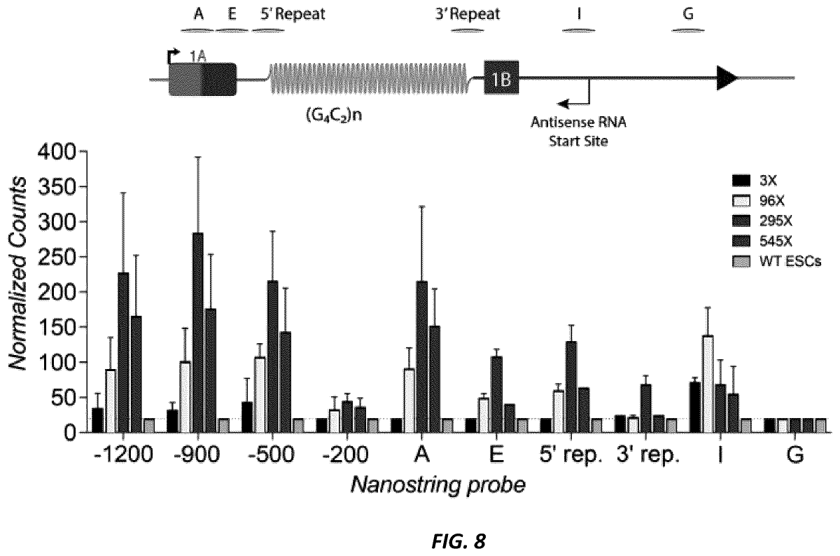

Mapping of the transcription start site (TSS) of the antisense transcripts produced by a humanized mouse C9orf72 alleles by 5′-RACE revealed that all of the cDNA clones shared the same sequence, which mapped a single TSS at an adenosine 171 bp downstream of the 3′ end of the exon 1B coding DNA, approximately 270 bp downstream of the GGGGCC hexanucleotide repeat (SEQ ID NO: 100) expansion. To confirm this mapping and measure the abundance of the antisense produced from the TSS, a collection of strand-specific Nanostring® probes was employed to quantify C9orf72 antisense RNA. The probes were designed to hybridize to antisense RNA derived from different regions of the humanized alleles, near the mapped start site (probe I) and both upstream (probe G) and downstream of the start site (probes 3′-rep. 5′-rep, E and A) (FIG. 8). We also designed probes to recognize antisense RNAs that might extend from 200-1200 nucleotides upstream of the start site of mouse sense RNA exon 1A.

The Nanostring results revealed that in ES cell-derived motor neurons (ESC-MNs) with 96 hexanucleotide repeats (96X) or greater (295X and 545X), antisense RNA was produced from the mapped initiation site (probe I) and extends through the repeat expansion (probes 3′-rep. 5′-rep, E and A) and at least 1500 bp out into the mouse gene's 5′ flanking sequence. Consistent with the mapped start site, no antisense transcripts with probe G was detected. Some antisense transcription was detected at the initiation site (probe I) in the 3X control ESC-MNs, but no significant accumulation of transcripts that elongated into the 3X repeat or beyond. Therefore, productive antisense RNA transcription, that is the accumulation of elongated transcripts, required longer repeat expansions. Accumulation of the extended antisense RNAs is dependent on the humanized allele and repeat expansion greater than 3X.

The mapping of the antisense RNA TSS and the extension of the elongated transcripts serves as a guide to direct targeting of the antisense RNA destruction by, for example, siRNA-directed RNA interference or antisense oligonucleotide directed RNase H degradation. As the TSS mapped within the human inserted sequence in the humanized mouse alleles, it is highly likely that it is the genuine site used in human cells. Targeting human sequences that span between probe I at the TSS and probe A in exon 1A (i.e., nucleotides 5026-5607 of NG_031977 (SEQ ID NO: 15)) and between probe I at the TSS and probe E in exon 1A (i.e., nucleotides 5130-5607 of NG_031977 (SEQ ID NO: 15)) would be most likely to yield effective therapeutic agents. The Nanostring quantitative probing indicated that the antisense RNAs extend far out into the mouse C9orf72 gene's 5′ flanking sequence. As it is likely that similar extension occurs in human cells, it could be productive to target homologous sequences associated with the human C9orf72 gene.

Informal Sequence Listing

SEQ ID NO: 1
>NM_001256054.2 Homo sapiens C9orf72-SMCR8 complex subunit (C9orf72),
transcript variant 3, mRNA
ACGTAACCTACGGTGTCCCGCTAGGAAAGAGAGGTGCGTCAAACAGCGACAAGTTCCGCCCACGTAAAAG
ATGACGCTTGGTGTGTCAGCCGTCCCTGCTGCCCGGTTGCTTCTCTTTTGGGGGCGGGGTCTAGCAAGAG
CAGGTGTGGGTTTAGGAGATATCTCCGGAGCATTTGGATAATGTGACAGTTGGAATGCAGTGATGTCGAC
TCTTTGCCCACCGCCATCTCCAGCTGTTGCCAAGACAGAGATTGCTTTAAGTGGCAAATCACCTTTATTA
GCAGCTACTTTTGCTTACTGGGACAATATTCTTGGTCCTAGAGTAAGGCACATTTGGGCTCCAAAGACAG
AACAGGTACTTCTCAGTGATGGAGAAATAACTTTTCTTGCCAACCACACTCTAAATGGAGAAATCCTTCG
AAATGCAGAGAGTGGTGCTATAGATGTAAAGTTTTTTGTCTTGTCTGAAAAGGGAGTGATTATTGTTTCA
TTAATCTTTGATGGAAACTGGAATGGGGATCGCAGCACATATGGACTATCAATTATACTTCCACAGACAG
AACTTAGTTTCTACCTCCCACTTCATAGAGTGTGTGTTGATAGATTAACACATATAATCCGGAAAGGAAG
AATATGGATGCATAAGGAAAGACAAGAAAATGTCCAGAAGATTATCTTAGAAGGCACAGAGAGAATGGAA
GATCAGGGTCAGAGTATTATTCCAATGCTTACTGGAGAAGTGATTCCTGTAATGGAACTGCTTTCATCTA
TGAAATCACACAGTGTTCCTGAAGAAATAGATATAGCTGATACAGTACTCAATGATGATGATATTGGTGA
CAGCTGTCATGAAGGCTTTCTTCTCAATGCCATCAGCTCACACTTGCAAACCTGTGGCTGTTCCGTTGTA
GTAGGTAGCAGTGCAGAGAAAGTAAATAAGATAGTCAGAACATTATGCCTTTTTCTGACTCCAGCAGAGA
GAAAATGCTCCAGGTTATGTGAAGCAGAATCATCATTTAAATATGAGTCAGGGCTCTTTGTACAAGGCCT
GCTAAAGGATTCAACTGGAAGCTTTGTGCTGCCTTTCCGGCAAGTCATGTATGCTCCATATCCCACCACA
CACATAGATGTGGATGTCAATACTGTGAAGCAGATGCCACCCTGTCATGAACATATTTATAATCAGCGTA
GATACATGAGATCCGAGCTGACAGCCTTCTGGAGAGCCACTTCAGAAGAAGACATGGCTCAGGATACGAT
CATCTACACTGACGAAAGCTTTACTCCTGATTTGAATATTTTTCAAGATGTCTTACACAGAGACACTCTA
GTGAAAGCCTTCCTGGATCAGGTCTTTCAGCTGAAACCTGGCTTATCTCTCAGAAGTACTTTCCTTGCAC
AGTTTCTACTTGTCCTTCACAGAAAAGCCTTGACACTAATAAAATATATAGAAGACGATACGCAGAAGGG
AAAAAAGCCCTTTAAATCTCTTCGGAACCTGAAGATAGACCTTGATTTAACAGCAGAGGGCGATCTTAAC
ATAATAATGGCTCTGGCTGAGAAAATTAAACCAGGCCTACACTCTTTTATCTTTGGAAGACCTTTCTACA
CTAGTGTGCAAGAACGAGATGTTCTAATGACTTTTTAAATGTGTAACTTAATAAGCCTATTCCATCACAA
TCATGATCGCTGGTAAAGTAGCTCAGTGGTGTGGGGAAACGTTCCCCTGGATCATACTCCAGAATTCTGC
TCTCAGCAATTGCAGTTAAGTAAGTTACACTACAGTTCTCACAAGAGCCTGTGAGGGGATGTCAGGTGCA
TCATTACATTGGGTGTCTCTTTTCCTAGATTTATGCTTTTGGGATACAGACCTATGTTTACAATATAATA
AATATTATTGCTATCTTTTAAAGATATAATAATAGGATGTAAACTTGACCACAACTACTGTTTTTTTGAA
ATACATGATTCATGGTTTACATGTGTCAAGGTGAAATCTGAGTTGGCTTTTACAGATAGTTGACTTTCTA
TCTTTTGGCATTCTTTGGTGTGTAGAATTACTGTAATACTTCTGCAATCAACTGAAAACTAGAGCCTTTA
AATGATTTCAATTCCACAGAAAGAAAGTGAGCTTGAACATAGGATGAGCTTTAGAAAGAAAATTGATCAA
GCAGATGTTTAATTGGAATTGATTATTAGATCCTACTTTGTGGATTTAGTCCCTGGGATTCAGTCTGTAG
AAATGTCTAATAGTTCTCTATAGTCCTTGTTCCTGGTGAACCACAGTTAGGGTGTTTTGTTTATTTTATT
GTTCTTGCTATTGTTGATATTCTATGTAGTTGAGCTCTGTAAAAGGAAATTGTATTTTATGTTTTAGTAA
TTGTTGCCAACTTTTTAAATTAATTTTCATTATTTTTGAGCCAAATTGAAATGTGCACCTCCTGTGCCTT
TTTTCTCCTTAGAAAATCTAATTACTTGGAACAAGTTCAGATTTCACTGGTCAGTCATTTTCATCTTGTT
TTCTTCTTGCTAAGTCTTACCATGTACCTGCTTTGGCAATCATTGCAACTCTGAGATTATAAAATGCCTT
AGAGAATATACTAACTAATAAGATCTTTTTTTCAGAAACAGAAAATAGTTCCTTGAGTACTTCCTTCTTG
CATTTCTGCCTATGTTTTTGAAGTTGTTGCTGTTTGCCTGCAATAGGCTATAAGGAATAGCAGGAGAAAT
TTTACTGAAGTGCTGTTTTCCTAGGTGCTACTTTGGCAGAGCTAAGTTATCTTTTGTTTTCTTAATGCGT
TTGGACCATTTTGCTGGCTATAAAATAACTGATTAATATAATTCTAACACAATGTTGACATTGTAGTTAC
ACAAACACAAATAAATATTTTATTTAAAATTCTGGAAGTAATATAAAAGGGAAAATATATTTATAAGAAA
GGGATAAAGGTAATAGAGCCCTTCTGCCCCCCACCCACCAAATTTACACAACAAAATGACATGTTCGAAT
GTGAAAGGTCATAATAGCTTTCCCATCATGAATCAGAAAGATGTGGACAGCTTGATGTTTTAGACAACCA
CTGAACTAGATGACTGTTGTACTGTAGCTCAGTCATTTAAAAAATATATAAATACTACCTTGTAGTGTCC
CATACTGTGTTTTTTACATGGTAGATTCTTATTTAAGTGCTAACTGGTTATTTTCTTTGGCTGGTTTATT
GTACTGTTATACAGAATGTAAGTTGTACAGTGAAATAAGTTATTAAAGCATGTGTAAACATTGTTATATA
TCTTTTCTCCTAAATGGAGAATTTTGAATAAAATATATTTGAAATTTTAAAAAAAAAAAAAAAAAA
SEQ ID NO: 2
>XM_005581570.2 PREDICTED: Macaca fascicularis chromosome 15 open reading
frame, huma C9orf72 (C15H9orf72), transcript variant X2, mRNA
ACGTAACCTACGGTGTCCCGCTAGGAAAGAGAGGCGCGTCAAACAGCGACAAGTTCCGCCCACGTAAAAG
ATGACGCTTGGTGCGTCAGCCGTCCCTGCTGCCCGGTTCCTTCTCTCTGGGGGCGGGGCCTGGCTAGAGC
AGGTGTGGGTTTAGGAGATATCTCAGGAGCATTTGGATAATGTGACAGTTGGAATGCAGTGATGTCGACT
CTTTGCCCACCGCCATCTCCAGCTGTTGCCAAGACAGAGATTGCTTTAAGTGGTGAATCACCTTTATTAG
CAGCTACTTTTGCTTACTGGGACAATATTCTTGGTCCTAGAGTAAGGCACATTTGGGCTCCAAAGACAGA
ACAGGTACTTCTCAGTGACGGAGAAATAACTTTTCTTGCCAACCACACTCTAAATGGAGAAATCCTTCGA
AATGCAGAGAGTGGTGCTATAGATGTAAAGTTTTTTGTCTTGTCTGAAAAGGGAGTGATTATTGTTTCAT
TAATCTTTGATGGAAACTGGAATGGGGATCGCAGCACATACGGACTATCAATTATACTTCCACAGACAGA
ACTTAGTTTCTACCTCCCACTTCATAGAGTGTGTGTTGATAGATTAACACATATAATCCGGAAAGGAAGA
ATATGGATGCATAAGGAAAGACAAGAAAATGTCCAGAAGATTATCTTAGAAGGCACAGAGAGAATGGAAG
ATCAGGGTCAGAGTATTATTCCAATGCTTACTGGAGAAGTGATTCCTGTAATGGAACTGCTTTCATCTAT
GAAATCACACAGTGTTCCTGAAGAAATAGATATAGCTGATACAGTACTCAATGATGATGATATTGGTGAC
AGTTGTCATGAAGGCTTTCTTCTCAATGCCATCAGCTCACACTTGCAAACCTGTGGCTGTTCCGTTGTAG
TAGGTAGCAGTGCAGAGAAAGTAAATAAGATAGTCAGAACATTATGCCTTTTTCTGACTGCAGCAGAGAG
AAAATGCTCCAGGTTATGTGAAGCAGAATCATCATTTAAATATGAGTCAGGGCTCTTTGTACAGGGCCTG
CTAAAGGATTCAACTGGAAGCTTTGTGCTGCCTTTCCGGCAAGTCATGTATGCTCCATATCCCACCACAC
ACATAGATGTGGATGTCAATACTGTGAAGCAGATGCCACCCTGTCATGAACATATTTATAATCAGCGTAG
ATACATGAGATCCGAGCTGACAGCCTTCTGGAGAGCCACTTCAGAAGAAGACATGGCTCAGGATACGATC
ATCTACACTGACGAAAGCTTTACTCCTGATTTGAATATTTTTCAAGATGTCTTACACAGAGACACTCTAG
TGAAAGCCTTCCTGGATCAGGTCTTTCAGCTGAAACCTGGCTTATCTCTCAGGAGTACTTTCCTTGCACA
GTTTTTACTTGTCCTTCACAGAAAAGCCTTGACACTAATAAAATATATAGAAGATGATACGCAGAAGGGA
AAAAAGCCCTTTAAATCTCTTCGGAACCTGAAGATAGACCTTGATTTAACAGCAGAGGGCGATCTTAACA
TAATAATGGCTCTGGCTGAGAAAATTAAACCAGGCCTACACTCTTTTATCTTTGGAAGACCTTTCTACAC
TAGTGTACAAGAACGAGATGTTCTAATGACTTTTTAAATGTGTAACTTAATAAGCCTATTCCATCACAAT
CGTGATCGCTGCTAAAGTAGCTCGGTGGTGTGGGGAAACATTCCCCTGGATCATACTCCAGAGCTCTGCT
CGGCAGTTGCAGTTAAGTTAGTTACACTACAGTTCTCACAAGAGTCTGTGAGGGGATGTCAGGTGCATCA
TTACATTGGATGTCTCTTTTCCTAGATTTATGCTTTTGGGATACAGACCTATGTTTACAATATAATAGGT
ATTATTGCTGTCTTTTAAATATATAATAATAGGATATAAACTTGACCACAACTGCTGTTTTTTTGAAATA
TATGATTCATGGTTTACATGTATTAAGGTGAAATCCGAGTTCGCTTTTACAGATATTAGTTGACTTTCTA
TCTTTTGGCATTCTTTGGTGTGTGGAATTACTGTAATACTTCTGCAATCAACTGAAAATTAGAGCCTTTA
AATGATTTCAGTTCCACAGAAAGAAAGTGAGCTTCAACATAGGATAAGCTTTAGAAAGAGAATTGATCAA
GCAGATGTTTAATTGGAATTGATTATTAGATCCTGCTTTGTGGATTTAGCCCTCGGGATTCAGTCTGTAG
AAATGTCTGATAGTTCTCTATAGTCCCTGCTCATGGTGAACCACAGTTAGGATGTTTTGTTTGTTTTATT
GTTGTTGCTATTGTTGATGTTCTATATAGTTGAGCTCTGTAAAAGGAAATTGTATTTTATGTTTTAGTAG
TTGTTGCCAACTTTTTAAATTAATTTTCATTATTTTTGAGCCAAATTGAAATGTGCACCTCCTGTGCCTT
TTTTTTCCTTGGAAAATCGAATTACTTGGAAGAAGTTCAGATTTCACTGGTCAGTCGTTTTCATCTTGTT
TTCTTCTTGCAGAGTCTTACCATGTACCTGCTTTGGCAATCATTGTAACTCTGAGATTATAAAATGCATT
AGAGAATATATTAACTAATAAGATCTTTTTTTTCAGGAACAGAAAATAGTTCCTTGAGTACTTCCTTCTT
ACATTTCTGCCCATGTTTTTGAAGTTGTTGCCATTTGCCTGCAATAGGCTATAAGGAATAGCAGGAGAAA
TTTTACTGAAGTGCTATTTTTCTAGGTGCTACTTTGGCAGAGCTAAGTGGTCTGTTTCTTTTGTTTCCTT
AATGCGTTTGGACCATTTTGCTGGCTGTAAAATAACTGATTAATATAATTCTAACACAATATTGACATTG
TAGTGTACACAAACACAAATATTTTATTTAAAACTGGAAGTAACATAAAAGGGAAAATATATTTATAAGA
AAGGAATAAAGGTAATAGAGCTCTTCTGTCCCCCAGCCACCAAATTTACACAACAAAATGATATGTTCTA
ATGTGAAAGGTCATAATAGCTTTCCCATCATTAATCAGAAAGATGTGGCAGCTTGATTTTTCAGACAACC
CCTGAACTAGATGACTGTTGTACTGTAGCTCAGTCATTTAAAAAATATATAAATACTATCTCGTAGTGTC
CCATACTATGTTTTTTACATGATAGATTCTTATTTAAGTGCTACCTGGTTATTTTCTTTGGCTGGTTTAT
TGTACTGTTATATAGAATGTAAGTTGTACAGTGAAATAAGTTATTAAAGCATGTGTAAACATTGTTATAT
ATCTTTTCTCCTAGATGGAGAATTTTGAATAAAATATATTTGAAATTTT
SEQ ID NO: 3
>NM_001081343.2 Mus musculus C9orf72, member of C9orf72-SMCR8 complex
(C9orf72), transcript variant 1, mRNA
GCGGTTGCGGTCCCTGCGCCGGCGGTGAAGGCGCAGCAGCGGCGAGTGGCTATTGCAAGCGTTCGGATAA
TGTGAGACCTGGAATGCAGTGAGACCTGGGATGCAGGGATGTCGACTATCTGCCCCCCACCATCTCCTGC
TGTTGCCAAGACAGAGATTGCTTTAAGTGGTGAATCACCCTTGTTGGCGGCTACCTTTGCTTACTGGGAT
AATATTCTTGGTCCTAGAGTAAGGCATATTTGGGCTCCAAAGACAGACCAAGTGCTTCTCAGTGATGGAG
AAATAACTTTTCTTGCCAACCACACTCTAAATGGAGAAATTCTTCGAAATGCAGAGAGTGGGGCTATAGA
TGTAAAATTTTTTGTCTTATCTGAAAAAGGGGTAATTATTGTTTCATTAATCTTCGACGGAAACTGGAAT
GGAGATCGGAGCACTTATGGACTATCAATTATACTGCCGCAGACAGAGCTGAGCTTCTACCTCCCACTTC
ACAGAGTGTGTGTTGACAGGCTAACACACATTATTCGAAAAGGAAGAATATGGATGCATAAGGAAAGACA
AGAAAATGTCCAGAAAATTGTCTTGGAAGGCACAGAGAGGATGGAAGATCAGGGTCAGAGTATCATTCCC
ATGCTTACTGGGGAAGTCATTCCTGTAATGGAGCTGCTTGCATCTATGAAATCCCACAGTGTTCCTGAAG
ACATTGATATAGCTGATACAGTGCTCAATGATGATGACATTGGTGACAGCTGTCACGAAGGCTTTCTTCT
CAATGCCATCAGCTCACACCTGCAGACCTGTGGCTGTTCCGTTGTAGTTGGCAGCAGTGCAGAGAAAGTA
AATAAGATAGTAAGAACGCTGTGCCTTTTTCTGACACCAGCAGAGAGGAAATGCTCCAGGCTGTGTGAAG
CAGAATCGTCCTTTAAGTACGAATCGGGACTCTTTGTGCAAGGCTTGCTAAAGGATGCAACAGGCAGTTT
TGTCCTACCCTTCCGGCAAGTTATGTATGCCCCGTACCCCACCACGCACATTGATGTGGATGTCAACACT
GTCAAGCAGATGCCACCGTGTCATGAACATATTTATAATCAACGCAGATACATGAGGTCAGAGCTGACAG
CCTTCTGGAGGGCAACTTCAGAAGAGGACATGGCGCAGGACACCATCATCTACACAGATGAGAGCTTCAC
TCCTGATTTGAATATTTTCCAAGATGTCTTACACAGAGACACTCTAGTGAAAGCCTTCCTGGATCAGGTC
TTCCATTTGAAGCCTGGCCTGTCTCTCAGGAGTACTTTCCTTGCACAGTTCCTCCTCATTCTTCACAGAA
AAGCCTTGACACTAATCAAGTACATCGAGGATGATACGCAGAAGGGGAAAAAGCCCTTTAAGTCTCTTCG
GAACCTGAAGATAGATCTTGATTTAACAGCAGAGGGCGATCTTAACATAATAATGGCTCTAGCTGAGAAA
ATTAAGCCAGGCCTACACTCTTTCATCTTTGGGAGACCTTTCTACACTAGTGTACAAGAACGTGATGTTC
TAATGACCTTTTGACCGTGTGGTTTGCTGTGTCTGTCTCTTCACAGTCACACCTGCTGTTACAGTGTCTC
AGCAGTGTGTGGGCACATCCTTCCTCCCGAGTCCTGCTGCAGGACAGGGTACACTACACTTGTCAGTAGA
AGTCTGTACCTGATGTCAGGTGCATCGTTACAGTGAATGACTCTTCCTAGAATAGATGTACTCTTTTAGG
GCCTTATGTTTACAATTATCCTAAGTACTATTGCTGTCTTTTAAAGATATGAATGATGGAATATACACTT
GACCATAACTGCTGATTGGTTTTTTGTTTTGTTTTGTTTGTTTTCTTGGAAACTTATGATTCCTGGTTTA
CATGTACCACACTGAAACCCTCGTTAGCTTTACAGATAAAGTGTGAGTTGACTTCCTGCCCCTCTGTGTT
CTGTGGTATGTCCGATTACTTCTGCCACAGCTAAACATTAGAGCATTTAAAGTTTGCAGTTCCTCAGAAA
GGAACTTAGTCTGACTACAGATTAGTTCTTGAGAGAAGACACTGATAGGGCAGAGCTGTAGGTGAAATCA
GTTGTTAGCCCTTCCTTTATAGACGTAGTCCTTCAGATTCGGTCTGTACAGAAATGCCGAGGGGTCATGC
ATGGGCCCTGAGTATCGTGACCTGTGACAAGTTTTTTGTTGGTTTATTGTAGTTCTGTCAAAGAAAGTGG
CATTTGTTTTTATAATTGTTGCCAACTTTTAAGGTTAATTTTCATTATTTTTGAGCCGAATTAAAATGCG
CACCTCCTGTGCCTTTCCCAATCTTGGAAAATATAATTTCTTGGCAGAGGGTCAGATTTCAGGGCCCAGT
CACTTTCATCTGACCACCCTTTGCACGGCTGCCGTGTGCCTGGCTTAGATTAGAAGTCCTTGTTAAGTAT
GTCAGAGTACATTCGCTGATAAGATCTTTGAAGAGCAGGGAAGCGTCTTGCCTCTTTCCTTTGGTTTCTG
CCTGTACTCTGGTGTTTCCCGTGTCACCTGCATCATAGGAACAGCAGAGAAATCTGACCCAGTGCTATTT
TTCTAGGTGCTACTATGGCAAACTCAAGTGGTCTGTTTCTGTTCCTGTAACGTTCGACTATCTCGCTAGC
TGTGAAGTACTGATTAGTGGAGTTCTGTGCAACAGCAGTGTAGGAGTATACACAAACACAAATATGTGTT
TCTATTTAAAACTGTGGACTTAGCATAAAAAGGGAGAATATATTTATTTTTTACAAAAGGGATAAAAATG
GGCCCCGTTCCTCACCCACCAGATTTAGCGAGAAAAAGCTTTCTATTCTGAAAGGTCACGGTGGCTTTGG
CATTACAAATCAGAACAACACACACTGACCATGATGGCTTGTGAACTAACTGCAAGGCACTCCGTCATGG
TAAGCGAGTAGGTCCCACCTCCTAGTGTGCCGCTCATTGCTTTACACAGTAGAATCTTATTTGAGTGCTA
ATTGTTGTCTTTGCTGCTTTACTGTGTTGTTATAGAAAATGTAAGCTGTACAGTGAATAAGTTATTGAAG
CATGTGTAAACACTGTTATATATCTTTTCTCCTAGATGGGGAATTTTGAATAAAATACCTTTGAAATTCT
G
SEQ ID NO: 4
>NM_001007702.1 Rattus norvegicus similar to RIKEN CDNA 3110043021
(RGD1359108), mRNA
CGTTTGTAGTGTCAGCCATCCCAATTGCCTGTTCCTTCTCTGTGGGAGTGGTGTCTAGACAGTCCAGGCA
GGGTATGCTAGGCAGGTGCGTTTTGGTTGCCTCAGATCGCAACTTGACTCCATAACGGTGACCAAAGACA
AAAGAAGGAAACCAGATTAAAAAGAACCGGACACAGACCCCTGCAGAATCTGGAGCGGCCGTGGTTGGGG
GCGGGGCTACGACGGGGCGGACTCGGGGGCGTGGGAGGGCGGGGCCGGGGCGGGGCCCGGAGCCGGCTGC
GGTTGCGGTCCCTGCGCCGGCGGTGAAGGCGCAGCGGCGGCGAGTGGCTATTGCAAGCGTTTGGATAATG
TGAGACCTGGGATGCAGGGATGTCGACTATCTGCCCCCCACCATCTCCTGCTGTTGCCAAGACAGAGATT
GCTTTAAGTGGTGAATCACCCTTGTTGGCGGCTACCTTTGCTTACTGGGATAATATTCTTGGTCCTAGAG
TAAGGCACATTTGGGCTCCAAAGACAGACCAAGTACTCCTCAGTGATGGAGAAATCACTTTTCTTGCCAA
CCACACTCTGAATGGAGAAATTCTTCGGAATGCGGAGAGTGGGGCAATAGATGTAAAGTTTTTTGTCTTA
TCTGAAAAGGGCGTCATTATTGTTTCATTAATCTTCGACGGGAACTGGAACGGAGATCGGAGCACTTACG
GACTATCAATTATACTGCCGCAGACGGAGCTGAGTTTCTACCTCCCACTGCACAGAGTGTGTGTTGACAG
GCTAACGCACATCATTCGAAAAGGAAGGATATGGATGCACAAGGAAAGACAAGAAAATGTCCAGAAAATT
GTCTTGGAAGGCACCGAGAGGATGGAAGATCAGGGTCAGAGTATCATCCCTATGCTTACTGGGGAGGTCA
TCCCTGTGATGGAGCTGCTTGCGTCTATGAGATCACACAGTGTTCCTGAAGACCTCGATATAGCTGATAC
AGTACTCAATGATGATGACATTGGTGACAGCTGTCATGAAGGCTTTCTTCTCAATGCCATCAGCTCACAT
CTGCAGACCTGCGGCTGTTCTGTGGTGGTAGGCAGCAGTGCAGAGAAAGTAAATAAGATAGTAAGAACAC
TGTGCCTTTTTCTGACACCAGCAGAGAGGAAGTGCTCCAGGCTGTGTGAAGCCGAATCGTCCTTTAAATA
CGAATCTGGACTCTTTGTACAAGGCTTGCTAAAGGATGCGACTGGCAGTTTTGTACTACCTTTCCGGCAA
GTTATGTATGCCCCTTATCCCACCACACACATCGATGTGGATGTCAACACTGTCAAGCAGATGCCACCGT
GTCATGAACATATTTATAATCAACGCAGATACATGAGGTCAGAGCTGACAGCCTTCTGGAGGGCAACTTC
AGAAGAGGACATGGCTCAGGACACCATCATCTACACAGATGAGAGCTTCACTCCTGATTTGAATATTTTC
CAAGATGTCTTACACAGAGACACTCTAGTGAAAGCCTTTCTGGATCAGGTCTTCCATTTGAAGCCTGGCC
TGTCTCTCAGGAGTACTTTCCTTGCACAGTTCCTCCTCATTCTTCACAGAAAAGCCTTGACACTAATCAA
GTACATAGAGGATGACACGCAGAAGGGGAAAAAGCCCTTTAAGTCTCTTCGGAACCTGAAGATAGATCTT
GATTTAACAGCAGAGGGCGACCTTAACATAATAATGGCTCTAGCTGAGAAAATTAAGCCAGGCCTACACT
CTTTCATCTTCGGGAGACCTTTCTACACTAGTGTCCAAGAACGTGATGTTCTAATGACTTTTTAAACATG
TGGTTTGCTCCGTGTGTCTCATGACAGTCACACTTGCTGTTACAGTGTCTCAGCGCTTTGGACACATCCT
TCCTCCAGGGTCCTGCCGCAGGACACGTTACACTACACTTGTCAGTAGAGGTCTGTACCAGATGTCAGGT
ACATCGTTGTAGTGAATGTCTCTTTTCCTAGACTAGATGTACCCTCGTAGGGACTTATGTTTACAACCCT
CCTAAGTACTAGTGCTGTCTTGTAAGGATACGAATGAAGGGATGTAAACTTCACCACAACTGCTGGTTGG
TTTTGTTGTTTTTGTTTTTTGAAACTTATAATTCATGGTTTACATGCATCACACTGAAACCCTAGTTAGC
TTTTTACAGGTAAGCTGTGAGTTGACTGCCTGTCCCTGTGTTCTCTGGCCTGTACGATCTGTGGCGTGTA
GGATCACTTTTGCAACAACTAAAAACTAAAGCACTTTGTTTGCAGTTCTACAGAAAGCAACTTAGTCTGT
CTGCAGATTCGTTTTTGAAAGAAGACATGAGAAAGCGGAGTTTTAGGTGAAGTCAGTTGTTGGATCTTCC
TTTATAGACTTAGTCCTTTAGATGTGGTCTGTATAGACATGCCCAACCATCATGCATGGGCACTGAATAT
CGTGAACTGTGGTATGCTTTTTGTTGGTTTATTGTACTTCTGTCAAAGAAAGTGGCATTGGTTTTTATAA
TTGTTGCCAAGTTTTAAGGTTAATTTTCATTATTTTTGAGCCAAATTAAAATGTGCACCTCCTGTGCCTT
TCCCAATCTTGGAAAATATAATTTCTTGGCAGAAGGTCAGATTTCAGGGCCCAGTCACTTTCGTCTGACT
TCCCTTTGCACAGTCCGCCATGGGCCTGGCTTAGAAGTTCTTGTAAACTATGCCAGAGAGTACATTCGCT
GATAAAATCTTCTTTGCAGAGCAGGAGAGCTTCTTGCCTCTTTCCTTTCATTTCTGCCTGGACTTTGGTG
TTCTCCACGTTCCCTGCATCCTAAGGACAGCAGGAGAACTCTGACCCCAGTGCTATTTCTCTAGGTGCTA
TTGTGGCAAACTCAAGCGGTCCGTCTCTGTCCCTGTAACGTTCGTACCTTGCTGGCTGTGAAGTACTGAC
TGGTAAAGCTCCGTGCTACAGCAGTGTAGGGTATACACAAACACAAGTAAGTGTTTTATTTAAAACTGTG
GACTTAGCATAAAAAGGGAGACTATATTTATTTTTTACAAAAGGGATAAAAATGGAACCCTTTCCTCACC
CACCAGATTTAGTCAGAAAAAAACATTCTATTCTGAAAGGTCACAGTGGTTTTGACATGACACATCAGAA
CAACGCACACTGTCCATGATGGCTTATGAACTCCAAGTCACTCCATCATGGTAAATGGGTAGATCCCTCC
TTCTAGTGTGCCACACCATTGCTTCCCACAGTAGAATCTTATTTAAGTGCTAAGTGTTGTCTCTGCTGGT
TTACTCTGTTGTTTTAGAGAATGTAAGTTGTATAGTGAATAAGTTATTGAAGCATGTGTAAACACTGTTA
TACATCTTTTCTCCTAGATGGGGAATTTGGAATAAAATACCTTTAAAATTCAAAAAAAAAAAAAAAAAAA
AAAAA
SEQ ID NO: 5
>Reverse Complement of SEQ ID NO: 1
TTTTTTTTTTTTTTTTTTAAAATTTCAAATATATTTTATTCAAAATTCTCCATTTAGGAGAAAAGATATATAACA
ATGTTTACACATGCTTTAATAACTTATTTCACTGTACAACTTACATTCTGTATAACAGTACAATAAACCAGCCAA
AGAAAATAACCAGTTAGCACTTAAATAAGAATCTACCATGTAAAAAACACAGTATGGGACACTACAAGGTAGTAT
TTATATATTTTTTAAATGACTGAGCTACAGTACAACAGTCATCTAGTTCAGTGGTTGTCTAAAACATCAAGCTGT
CCACATCTTTCTGATTCATGATGGGAAAGCTATTATGACCTTTCACATTCGAACATGTCATTTTGTTGTGTAAAT
TTGGTGGGTGGGGGGCAGAAGGGCTCTATTACCTTTATCCCTTTCTTATAAATATATTTTCCCTTTTATATTACT
TCCAGAATTTTAAATAAAATATTTATTTGTGTTTGTGTAACTACAATGTCAACATTGTGTTAGAATTATATTAAT
CAGTTATTTTATAGCCAGCAAAATGGTCCAAACGCATTAAGAAAACAAAAGATAACTTAGCTCTGCCAAAGTAGC
ACCTAGGAAAACAGCACTTCAGTAAAATTTCTCCTGCTATTCCTTATAGCCTATTGCAGGCAAACAGCAACAACT
TCAAAAACATAGGCAGAAATGCAAGAAGGAAGTACTCAAGGAACTATTTTCTGTTTCTGAAAAAAAGATCTTATT
AGTTAGTATATTCTCTAAGGCATTTTATAATCTCAGAGTTGCAATGATTGCCAAAGCAGGTACATGGTAAGACTT
AGCAAGAAGAAAACAAGATGAAAATGACTGACCAGTGAAATCTGAACTTGTTCCAAGTAATTAGATTTTCTAAGG
AGAAAAAAGGCACAGGAGGTGCACATTTCAATTTGGCTCAAAAATAATGAAAATTAATTTAAAAAGTTGGCAACA
ATTACTAAAACATAAAATACAATTTCCTTTTACAGAGCTCAACTACATAGAATATCAACAATAGCAAGAACAATA
AAATAAACAAAACACCCTAACTGTGGTTCACCAGGAACAAGGACTATAGAGAACTATTAGACATTTCTACAGACT
GAATCCCAGGGACTAAATCCACAAAGTAGGATCTAATAATCAATTCCAATTAAACATCTGCTTGATCAATTTTCT
TTCTAAAGCTCATCCTATGTTCAAGCTCACTTTCTTTCTGTGGAATTGAAATCATTTAAAGGCTCTAGTTTTCAG
TTGATTGCAGAAGTATTACAGTAATTCTACACACCAAAGAATGCCAAAAGATAGAAAGTCAACTATCTGTAAAAG
CCAACTCAGATTTCACCTTGACACATGTAAACCATGAATCATGTATTTCAAAAAAACAGTAGTTGTGGTCAAGTT
TACATCCTATTATTATATCTTTAAAAGATAGCAATAATATTTATTATATTGTAAACATAGGTCTGTATCCCAAAA
GCATAAATCTAGGAAAAGAGACACCCAATGTAATGATGCACCTGACATCCCCTCACAGGCTCTTGTGAGAACTGT
AGTGTAACTTACTTAACTGCAATTGCTGAGAGCAGAATTCTGGAGTATGATCCAGGGGAACGTTTCCCCACACCA
CTGAGCTACTTTACCAGCGATCATGATTGTGATGGAATAGGCTTATTAAGTTACACATTTAAAAAGTCATTAGAA
CATCTCGTTCTTGCACACTAGTGTAGAAAGGTCTTCCAAAGATAAAAGAGTGTAGGCCTGGTTTAATTTTCTCAG
CCAGAGCCATTATTATGTTAAGATCGCCCTCTGCTGTTAAATCAAGGTCTATCTTCAGGTTCCGAAGAGATTTAA
AGGGCTTTTTTCCCTTCTGCGTATCGTCTTCTATATATTTTATTAGTGTCAAGGCTTTTCTGTGAAGGACAAGTA
GAAACTGTGCAAGGAAAGTACTTCTGAGAGATAAGCCAGGTTTCAGCTGAAAGACCTGATCCAGGAAGGCTTTCA
CTAGAGTGTCTCTGTGTAAGACATCTTGAAAAATATTCAAATCAGGAGTAAAGCTTTCGTCAGTGTAGATGATCG
TATCCTGAGCCATGTCTTCTTCTGAAGTGGCTCTCCAGAAGGCTGTCAGCTCGGATCTCATGTATCTACGCTGAT
TATAAATATGTTCATGACAGGGTGGCATCTGCTTCACAGTATTGACATCCACATCTATGTGTGTGGTGGGATATG
GAGCATACATGACTTGCCGGAAAGGCAGCACAAAGCTTCCAGTTGAATCCTTTAGCAGGCCTTGTACAAAGAGCC
CTGACTCATATTTAAATGATGATTCTGCTTCACATAACCTGGAGCATTTTCTCTCTGCTGGAGTCAGAAAAAGGC
ATAATGTTCTGACTATCTTATTTACTTTCTCTGCACTGCTACCTACTACAACGGAACAGCCACAGGTTTGCAAGT
GTGAGCTGATGGCATTGAGAAGAAAGCCTTCATGACAGCTGTCACCAATATCATCATCATTGAGTACTGTATCAG
CTATATCTATTTCTTCAGGAACACTGTGTGATTTCATAGATGAAAGCAGTTCCATTACAGGAATCACTTCTCCAG
TAAGCATTGGAATAATACTCTGACCCTGATCTTCCATTCTCTCTGTGCCTTCTAAGATAATCTTCTGGACATTTT
CTTGTCTTTCCTTATGCATCCATATTCTTCCTTTCCGGATTATATGTGTTAATCTATCAACACACACTCTATGAA
GTGGGAGGTAGAAACTAAGTTCTGTCTGTGGAAGTATAATTGATAGTCCATATGTGCTGCGATCCCCATTCCAGT
TTCCATCAAAGATTAATGAAACAATAATCACTCCCTTTTCAGACAAGACAAAAAACTTTACATCTATAGCACCAC
TCTCTGCATTTCGAAGGATTTCTCCATTTAGAGTGTGGTTGGCAAGAAAAGTTATTTCTCCATCACTGAGAAGTA
CCTGTTCTGTCTTTGGAGCCCAAATGTGCCTTACTCTAGGACCAAGAATATTGTCCCAGTAAGCAAAAGTAGCTG
CTAATAAAGGTGATTTGCCACTTAAAGCAATCTCTGTCTTGGCAACAGCTGGAGATGGCGGTGGGCAAAGAGTCG
ACATCACTGCATTCCAACTGTCACATTATCCAAATGCTCCGGAGATATCTCCTAAACCCACACCTGCTCTTGCTA
GACCCCGCCCCCAAAAGAGAAGCAACCGGGCAGCAGGGACGGCTGACACACCAAGCGTCATCTTTTACGTGGGCG
GAACTTGTCGCTGTTTGACGCACCTCTCTTTCCTAGCGGGACACCGTAGGTTACGT
SEQ ID NO: 6
>Reverse Complement of SEQ ID NO: 2
AAAATTTCAAATATATTTTATTCAAAATTCTCCATCTAGGAGAAAAGATATATAACAATGTTTACACATGCTTTA
ATAACTTATTTCACTGTACAACTTACATTCTATATAACAGTACAATAAACCAGCCAAAGAAAATAACCAGGTAGC
ACTTAAATAAGAATCTATCATGTAAAAAACATAGTATGGGACACTACGAGATAGTATTTATATATTTTTTAAATG
ACTGAGCTACAGTACAACAGTCATCTAGTTCAGGGGTTGTCTGAAAAATCAAGCTGCCACATCTTTCTGATTAAT
GATGGGAAAGCTATTATGACCTTTCACATTAGAACATATCATTTTGTTGTGTAAATTTGGTGGCTGGGGGACAGA
AGAGCTCTATTACCTTTATTCCTTTCTTATAAATATATTTTCCCTTTTATGTTACTTCCAGTTTTAAATAAAATA
TTTGTGTTTGTGTACACTACAATGTCAATATTGTGTTAGAATTATATTAATCAGTTATTTTACAGCCAGCAAAAT
GGTCCAAACGCATTAAGGAAACAAAAGAAACAGACCACTTAGCTCTGCCAAAGTAGCACCTAGAAAAATAGCACT
TCAGTAAAATTTCTCCTGCTATTCCTTATAGCCTATTGCAGGCAAATGGCAACAACTTCAAAAACATGGGCAGAA
ATGTAAGAAGGAAGTACTCAAGGAACTATTTTCTGTTCCTGAAAAAAAAGATCTTATTAGTTAATATATTCTCTA
ATGCATTTTATAATCTCAGAGTTACAATGATTGCCAAAGCAGGTACATGGTAAGACTCTGCAAGAAGAAAACAAG
ATGAAAACGACTGACCAGTGAAATCTGAACTTCTTCCAAGTAATTCGATTTTCCAAGGAAAAAAAAGGCACAGGA
GGTGCACATTTCAATTTGGCTCAAAAATAATGAAAATTAATTTAAAAAGTTGGCAACAACTACTAAAACATAAAA
TACAATTTCCTTTTACAGAGCTCAACTATATAGAACATCAACAATAGCAACAACAATAAAACAAACAAAACATCC
TAACTGTGGTTCACCATGAGCAGGGACTATAGAGAACTATCAGACATTTCTACAGACTGAATCCCGAGGGCTAAA
TCCACAAAGCAGGATCTAATAATCAATTCCAATTAAACATCTGCTTGATCAATTCTCTTTCTAAAGCTTATCCTA
TGTTGAAGCTCACTTTCTTTCTGTGGAACTGAAATCATTTAAAGGCTCTAATTTTCAGTTGATTGCAGAAGTATT
ACAGTAATTCCACACACCAAAGAATGCCAAAAGATAGAAAGTCAACTAATATCTGTAAAAGCGAACTCGGATTTC
ACCTTAATACATGTAAACCATGAATCATATATTTCAAAAAAACAGCAGTTGTGGTCAAGTTTATATCCTATTATT
ATATATTTAAAAGACAGCAATAATACCTATTATATTGTAAACATAGGTCTGTATCCCAAAAGCATAAATCTAGGA
AAAGAGACATCCAATGTAATGATGCACCTGACATCCCCTCACAGACTCTTGTGAGAACTGTAGTGTAACTAACTT
AACTGCAACTGCCGAGCAGAGCTCTGGAGTATGATCCAGGGGAATGTTTCCCCACACCACCGAGCTACTTTAGCA
GCGATCACGATTGTGATGGAATAGGCTTATTAAGTTACACATTTAAAAAGTCATTAGAACATCTCGTTCTTGTAC
ACTAGTGTAGAAAGGTCTTCCAAAGATAAAAGAGTGTAGGCCTGGTTTAATTTTCTCAGCCAGAGCCATTATTAT
GTTAAGATCGCCCTCTGCTGTTAAATCAAGGTCTATCTTCAGGTTCCGAAGAGATTTAAAGGGCTTTTTTCCCTT
CTGCGTATCATCTTCTATATATTTTATTAGTGTCAAGGCTTTTCTGTGAAGGACAAGTAAAAACTGTGCAAGGAA
AGTACTCCTGAGAGATAAGCCAGGTTTCAGCTGAAAGACCTGATCCAGGAAGGCTTTCACTAGAGTGTCTCTGTG
TAAGACATCTTGAAAAATATTCAAATCAGGAGTAAAGCTTTCGTCAGTGTAGATGATCGTATCCTGAGCCATGTC
TTCTTCTGAAGTGGCTCTCCAGAAGGCTGTCAGCTCGGATCTCATGTATCTACGCTGATTATAAATATGTTCATG
ACAGGGTGGCATCTGCTTCACAGTATTGACATCCACATCTATGTGTGTGGTGGGATATGGAGCATACATGACTTG
CCGGAAAGGCAGCACAAAGCTTCCAGTTGAATCCTTTAGCAGGCCCTGTACAAAGAGCCCTGACTCATATTTAAA
TGATGATTCTGCTTCACATAACCTGGAGCATTTTCTCTCTGCTGCAGTCAGAAAAAGGCATAATGTTCTGACTAT
CTTATTTACTTTCTCTGCACTGCTACCTACTACAACGGAACAGCCACAGGTTTGCAAGTGTGAGCTGATGGCATT
GAGAAGAAAGCCTTCATGACAACTGTCACCAATATCATCATCATTGAGTACTGTATCAGCTATATCTATTTCTTC
AGGAACACTGTGTGATTTCATAGATGAAAGCAGTTCCATTACAGGAATCACTTCTCCAGTAAGCATTGGAATAAT
ACTCTGACCCTGATCTTCCATTCTCTCTGTGCCTTCTAAGATAATCTTCTGGACATTTTCTTGTCTTTCCTTATG
CATCCATATTCTTCCTTTCCGGATTATATGTGTTAATCTATCAACACACACTCTATGAAGTGGGAGGTAGAAACT
AAGTTCTGTCTGTGGAAGTATAATTGATAGTCCGTATGTGCTGCGATCCCCATTCCAGTTTCCATCAAAGATTAA
TGAAACAATAATCACTCCCTTTTCAGACAAGACAAAAAACTTTACATCTATAGCACCACTCTCTGCATTTCGAAG
GATTTCTCCATTTAGAGTGTGGTTGGCAAGAAAAGTTATTTCTCCGTCACTGAGAAGTACCTGTTCTGTCTTTGG
AGCCCAAATGTGCCTTACTCTAGGACCAAGAATATTGTCCCAGTAAGCAAAAGTAGCTGCTAATAAAGGTGATTC
ACCACTTAAAGCAATCTCTGTCTTGGCAACAGCTGGAGATGGCGGTGGGCAAAGAGTCGACATCACTGCATTCCA
ACTGTCACATTATCCAAATGCTCCTGAGATATCTCCTAAACCCACACCTGCTCTAGCCAGGCCCCGCCCCCAGAG
AGAAGGAACCGGGCAGCAGGGACGGCTGACGCACCAAGCGTCATCTTTTACGTGGGCGGAACTTGTCGCTGTTTG
ACGCGCCTCTCTTTCCTAGCGGGACACCGTAGGTTACGT
SEQ ID NO: 7
>Reverse Complement of SEQ ID NO: 123 SEQ ID NO: 3
CAGAATTTCAAAGGTATTTTATTCAAAATTCCCCATCTAGGAGAAAAGATATATAACAGTGTTTACACATGCTTC
AATAACTTATTCACTGTACAGCTTACATTTTCTATAACAACACAGTAAAGCAGCAAAGACAACAATTAGCACTCA
AATAAGATTCTACTGTGTAAAGCAATGAGCGGCACACTAGGAGGTGGGACCTACTCGCTTACCATGACGGAGTGC
CTTGCAGTTAGTTCACAAGCCATCATGGTCAGTGTGTGTTGTTCTGATTTGTAATGCCAAAGCCACCGTGACCTT
TCAGAATAGAAAGCTTTTTCTCGCTAAATCTGGTGGGTGAGGAACGGGGCCCATTTTTATCCCTTTTGTAAAAAA
TAAATATATTCTCCCTTTTTATGCTAAGTCCACAGTTTTAAATAGAAACACATATTTGTGTTTGTGTATACTCCT
ACACTGCTGTTGCACAGAACTCCACTAATCAGTACTTCACAGCTAGCGAGATAGTCGAACGTTACAGGAACAGAA
ACAGACCACTTGAGTTTGCCATAGTAGCACCTAGAAAAATAGCACTGGGTCAGATTTCTCTGCTGTTCCTATGAT
GCAGGTGACACGGGAAACACCAGAGTACAGGCAGAAACCAAAGGAAAGAGGCAAGACGCTTCCCTGCTCTTCAAA
GATCTTATCAGCGAATGTACTCTGACATACTTAACAAGGACTTCTAATCTAAGCCAGGCACACGGCAGCCGTGCA
AAGGGTGGTCAGATGAAAGTGACTGGGCCCTGAAATCTGACCCTCTGCCAAGAAATTATATTTTCCAAGATTGGG
AAAGGCACAGGAGGTGCGCATTTTAATTCGGCTCAAAAATAATGAAAATTAACCTTAAAAGTTGGCAACAATTAT
AAAAACAAATGCCACTTTCTTTGACAGAACTACAATAAACCAACAAAAAACTTGTCACAGGTCACGATACTCAGG
GCCCATGCATGACCCCTCGGCATTTCTGTACAGACCGAATCTGAAGGACTACGTCTATAAAGGAAGGGCTAACAA
CTGATTTCACCTACAGCTCTGCCCTATCAGTGTCTTCTCTCAAGAACTAATCTGTAGTCAGACTAAGTTCCTTTC
TGAGGAACTGCAAACTTTAAATGCTCTAATGTTTAGCTGTGGCAGAAGTAATCGGACATACCACAGAACACAGAG
GGGCAGGAAGTCAACTCACACTTTATCTGTAAAGCTAACGAGGGTTTCAGTGTGGTACATGTAAACCAGGAATCA
TAAGTTTCCAAGAAAACAAACAAAACAAAACAAAAAACCAATCAGCAGTTATGGTCAAGTGTATATTCCATCATT
CATATCTTTAAAAGACAGCAATAGTACTTAGGATAATTGTAAACATAAGGCCCTAAAAGAGTACATCTATTCTAG
GAAGAGTCATTCACTGTAACGATGCACCTGACATCAGGTACAGACTTCTACTGACAAGTGTAGTGTACCCTGTCC
TGCAGCAGGACTCGGGAGGAAGGATGTGCCCACACACTGCTGAGACACTGTAACAGCAGGTGTGACTGTGAAGAG
ACAGACACAGCAAACCACACGGTCAAAAGGTCATTAGAACATCACGTTCTTGTACACTAGTGTAGAAAGGTCTCC
CAAAGATGAAAGAGTGTAGGCCTGGCTTAATTTTCTCAGCTAGAGCCATTATTATGTTAAGATCGCCCTCTGCTG
TTAAATCAAGATCTATCTTCAGGTTCCGAAGAGACTTAAAGGGCTTTTTCCCCTTCTGCGTATCATCCTCGATGT
ACTTGATTAGTGTCAAGGCTTTTCTGTGAAGAATGAGGAGGAACTGTGCAAGGAAAGTACTCCTGAGAGACAGGC
CAGGCTTCAAATGGAAGACCTGATCCAGGAAGGCTTTCACTAGAGTGTCTCTGTGTAAGACATCTTGGAAAATAT
TCAAATCAGGAGTGAAGCTCTCATCTGTGTAGATGATGGTGTCCTGCGCCATGTCCTCTTCTGAAGTTGCCCTCC
AGAAGGCTGTCAGCTCTGACCTCATGTATCTGCGTTGATTATAAATATGTTCATGACACGGTGGCATCTGCTTGA
CAGTGTTGACATCCACATCAATGTGCGTGGTGGGGTACGGGGCATACATAACTTGCCGGAAGGGTAGGACAAAAC
TGCCTGTTGCATCCTTTAGCAAGCCTTGCACAAAGAGTCCCGATTCGTACTTAAAGGACGATTCTGCTTCACACA
GCCTGGAGCATTTCCTCTCTGCTGGTGTCAGAAAAAGGCACAGCGTTCTTACTATCTTATTTACTTTCTCTGCAC
TGCTGCCAACTACAACGGAACAGCCACAGGTCTGCAGGTGTGAGCTGATGGCATTGAGAAGAAAGCCTTCGTGAC
AGCTGTCACCAATGTCATCATCATTGAGCACTGTATCAGCTATATCAATGTCTTCAGGAACACTGTGGGATTTCA
TAGATGCAAGCAGCTCCATTACAGGAATGACTTCCCCAGTAAGCATGGGAATGATACTCTGACCCTGATCTTCCA
TCCTCTCTGTGCCTTCCAAGACAATTTTCTGGACATTTTCTTGTCTTTCCTTATGCATCCATATTCTTCCTTTTC
GAATAATGTGTGTTAGCCTGTCAACACACACTCTGTGAAGTGGGAGGTAGAAGCTCAGCTCTGTCTGCGGCAGTA
TAATTGATAGTCCATAAGTGCTCCGATCTCCATTCCAGTTTCCGTCGAAGATTAATGAAACAATAATTACCCCTT
TTTCAGATAAGACAAAAAATTTTACATCTATAGCCCCACTCTCTGCATTTCGAAGAATTTCTCCATTTAGAGTGT
GGTTGGCAAGAAAAGTTATTTCTCCATCACTGAGAAGCACTTGGTCTGTCTTTGGAGCCCAAATATGCCTTACTC
TAGGACCAAGAATATTATCCCAGTAAGCAAAGGTAGCCGCCAACAAGGGTGATTCACCACTTAAAGCAATCTCTG
TCTTGGCAACAGCAGGAGATGGTGGGGGGCAGATAGTCGACATCCCTGCATCCCAGGTCTCACTGCATTCCAGGT
CTCACATTATCCGAACGCTTGCAATAGCCACTCGCCGCTGCTGCGCCTTCACCGCCGGCGCAGGGACCGCAACCG
C
SEQ ID NO: 8
>Reverse Complement of SEQ ID NO: 4
TTTTTTTTTTTTTTTTTTTTTTTTGAATTTTAAAGGTATTTTATTCCAAATTCCCCATCTAGGAGAAAAGATGTA
TAACAGTGTTTACACATGCTTCAATAACTTATTCACTATACAACTTACATTCTCTAAAACAACAGAGTAAACCAG
CAGAGACAACACTTAGCACTTAAATAAGATTCTACTGTGGGAAGCAATGGTGTGGCACACTAGAAGGAGGGATCT
ACCCATTTACCATGATGGAGTGACTTGGAGTTCATAAGCCATCATGGACAGTGTGCGTTGTTCTGATGTGTCATG
TCAAAACCACTGTGACCTTTCAGAATAGAATGTTTTTTTCTGACTAAATCTGGTGGGTGAGGAAAGGGTTCCATT
TTTATCCCTTTTGTAAAAAATAAATATAGTCTCCCTTTTTATGCTAAGTCCACAGTTTTAAATAAAACACTTACT
TGTGTTTGTGTATACCCTACACTGCTGTAGCACGGAGCTTTACCAGTCAGTACTTCACAGCCAGCAAGGTACGAA
CGTTACAGGGACAGAGACGGACCGCTTGAGTTTGCCACAATAGCACCTAGAGAAATAGCACTGGGGTCAGAGTTC
TCCTGCTGTCCTTAGGATGCAGGGAACGTGGAGAACACCAAAGTCCAGGCAGAAATGAAAGGAAAGAGGCAAGAA
GCTCTCCTGCTCTGCAAAGAAGATTTTATCAGCGAATGTACTCTCTGGCATAGTTTACAAGAACTTCTAAGCCAG
GCCCATGGCGGACTGTGCAAAGGGAAGTCAGACGAAAGTGACTGGGCCCTGAAATCTGACCTTCTGCCAAGAAAT
TATATTTTCCAAGATTGGGAAAGGCACAGGAGGTGCACATTTTAATTTGGCTCAAAAATAATGAAAATTAACCTT
AAAACTTGGCAACAATTATAAAAACCAATGCCACTTTCTTTGACAGAAGTACAATAAACCAACAAAAAGCATACC
ACAGTTCACGATATTCAGTGCCCATGCATGATGGTTGGGCATGTCTATACAGACCACATCTAAAGGACTAAGTCT
ATAAAGGAAGATCCAACAACTGACTTCACCTAAAACTCCGCTTTCTCATGTCTTCTTTCAAAAACGAATCTGCAG
ACAGACTAAGTTGCTTTCTGTAGAACTGCAAACAAAGTGCTTTAGTTTTTAGTTGTTGCAAAAGTGATCCTACAC
GCCACAGATCGTACAGGCCAGAGAACACAGGGACAGGCAGTCAACTCACAGCTTACCTGTAAAAAGCTAACTAGG
GTTTCAGTGTGATGCATGTAAACCATGAATTATAAGTTTCAAAAAACAAAAACAACAAAACCAACCAGCAGTTGT
GGTGAAGTTTACATCCCTTCATTCGTATCCTTACAAGACAGCACTAGTACTTAGGAGGGTTGTAAACATAAGTCC
CTACGAGGGTACATCTAGTCTAGGAAAAGAGACATTCACTACAACGATGTACCTGACATCTGGTACAGACCTCTA
CTGACAAGTGTAGTGTAACGTGTCCTGCGGCAGGACCCTGGAGGAAGGATGTGTCCAAAGCGCTGAGACACTGTA
ACAGCAAGTGTGACTGTCATGAGACACACGGAGCAAACCACATGTTTAAAAAGTCATTAGAACATCACGTTCTTG
GACACTAGTGTAGAAAGGTCTCCCGAAGATGAAAGAGTGTAGGCCTGGCTTAATTTTCTCAGCTAGAGCCATTAT
TATGTTAAGGTCGCCCTCTGCTGTTAAATCAAGATCTATCTTCAGGTTCCGAAGAGACTTAAAGGGCTTTTTCCC
CTTCTGCGTGTCATCCTCTATGTACTTGATTAGTGTCAAGGCTTTTCTGTGAAGAATGAGGAGGAACTGTGCAAG
GAAAGTACTCCTGAGAGACAGGCCAGGCTTCAAATGGAAGACCTGATCCAGAAAGGCTTTCACTAGAGTGTCTCT
GTGTAAGACATCTTGGAAAATATTCAAATCAGGAGTGAAGCTCTCATCTGTGTAGATGATGGTGTCCTGAGCCAT
GTCCTCTTCTGAAGTTGCCCTCCAGAAGGCTGTCAGCTCTGACCTCATGTATCTGCGTTGATTATAAATATGTTC
ATGACACGGTGGCATCTGCTTGACAGTGTTGACATCCACATCGATGTGTGTGGTGGGATAAGGGGCATACATAAC
TTGCCGGAAAGGTAGTACAAAACTGCCAGTCGCATCCTTTAGCAAGCCTTGTACAAAGAGTCCAGATTCGTATTT
AAAGGACGATTCGGCTTCACACAGCCTGGAGCACTTCCTCTCTGCTGGTGTCAGAAAAAGGCACAGTGTTCTTAC
TATCTTATTTACTTTCTCTGCACTGCTGCCTACCACCACAGAACAGCCGCAGGTCTGCAGATGTGAGCTGATGGC
ATTGAGAAGAAAGCCTTCATGACAGCTGTCACCAATGTCATCATCATTGAGTACTGTATCAGCTATATCGAGGTC
TTCAGGAACACTGTGTGATCTCATAGACGCAAGCAGCTCCATCACAGGGATGACCTCCCCAGTAAGCATAGGGAT
GATACTCTGACCCTGATCTTCCATCCTCTCGGTGCCTTCCAAGACAATTTTCTGGACATTTTCTTGTCTTTCCTT
GTGCATCCATATCCTTCCTTTTCGAATGATGTGCGTTAGCCTGTCAACACACACTCTGTGCAGTGGGAGGTAGAA
ACTCAGCTCCGTCTGCGGCAGTATAATTGATAGTCCGTAAGTGCTCCGATCTCCGTTCCAGTTCCCGTCGAAGAT
TAATGAAACAATAATGACGCCCTTTTCAGATAAGACAAAAAACTTTACATCTATTGCCCCACTCTCCGCATTCCG
AAGAATTTCTCCATTCAGAGTGTGGTTGGCAAGAAAAGTGATTTCTCCATCACTGAGGAGTACTTGGTCTGTCTT
TGGAGCCCAAATGTGCCTTACTCTAGGACCAAGAATATTATCCCAGTAAGCAAAGGTAGCCGCCAACAAGGGTGA
TTCACCACTTAAAGCAATCTCTGTCTTGGCAACAGCAGGAGATGGTGGGGGGCAGATAGTCGACATCCCTGCATC
CCAGGTCTCACATTATCCAAACGCTTGCAATAGCCACTCGCCGCCGCTGCGCCTTCACCGCCGGCGCAGGGACCG
CAACCGCAGCCGGCTCCGGGCCCCGCCCCGGCCCCGCCCTCCCACGCCCCCGAGTCCGCCCCGTCGTAGCCCCGC
CCCCAACCACGGCCGCTCCAGATTCTGCAGGGGTCTGTGTCCGGTTCTTTTTAATCTGGTTTCCTTCTTTTGTCT
TTGGTCACCGTTATGGAGTCAAGTTGCGATCTGAGGCAACCAAAACGCACCTGCCTAGCATACCCTGCCTGGACT
GTCTAGACACCACTCCCACAGAGAAGGAACAGGCAATTGGGATGGCTGACACTACAAACG
SEQ ID NO: 9
>NM_145005.6 Homo sapiens C9orf72-SMCR8 complex subunit (C9orf72) ,
transcript variant 1, mRNA
ACGTAACCTACGGTGTCCCGCTAGGAAAGAGAGGTGCGTCAAACAGCGACAAGTTCCGCCCACGTAAAAG
ATGACGCTTGATATCTCCGGAGCATTTGGATAATGTGACAGTTGGAATGCAGTGATGTCGACTCTTTGCC
CACCGCCATCTCCAGCTGTTGCCAAGACAGAGATTGCTTTAAGTGGCAAATCACCTTTATTAGCAGCTAC
TTTTGCTTACTGGGACAATATTCTTGGTCCTAGAGTAAGGCACATTTGGGCTCCAAAGACAGAACAGGTA
CTTCTCAGTGATGGAGAAATAACTTTTCTTGCCAACCACACTCTAAATGGAGAAATCCTTCGAAATGCAG
AGAGTGGTGCTATAGATGTAAAGTTTTTTGTCTTGTCTGAAAAGGGAGTGATTATTGTTTCATTAATCTT
TGATGGAAACTGGAATGGGGATCGCAGCACATATGGACTATCAATTATACTTCCACAGACAGAACTTAGT
TTCTACCTCCCACTTCATAGAGTGTGTGTTGATAGATTAACACATATAATCCGGAAAGGAAGAATATGGA
TGCATAAGGAAAGACAAGAAAATGTCCAGAAGATTATCTTAGAAGGCACAGAGAGAATGGAAGATCAGGG
TCAGAGTATTATTCCAATGCTTACTGGAGAAGTGATTCCTGTAATGGAACTGCTTTCATCTATGAAATCA
CACAGTGTTCCTGAAGAAATAGATATAGCTGATACAGTACTCAATGATGATGATATTGGTGACAGCTGTC
ATGAAGGCTTTCTTCTCAAGTAAGAATTTTTCTTTTCATAAAAGCTGGATGAAGCAGATACCATCTTATG
CTCACCTATGACAAGATTTGGAAGAAAGAAAATAACAGACTGTCTACTTAGATTGTTCTAGGGACATTAC
GTATTTGAACTGTTGCTTAAATTTGTGTTATTTTTCACTCATTATATTTCTATATATATTTGGTGTTATT
CCATTTGCTATTTAAAGAAACCGAGTTTCCATCCCAGACAAGAAATCATGGCCCCTTGCTTGATTCTGGT
TTCTTGTTTTACTTCTCATTAAAGCTAACAGAATCCTTTCATATTAAGTTGTACTGTAGATGAACTTAAG
TTATTTAGGCGTAGAACAAAATTATTCATATTTATACTGATCTTTTTCCATCCAGCAGTGGAGTTTAGTA
CTTAAGAGTTTGTGCCCTTAAACCAGACTCCCTGGATTAATGCTGTGTACCCGTGGGCAAGGTGCCTGAA
TTCTCTATACACCTATTTCCTCATCTGTAAAATGGCAATAATAGTAATAGTACCTAATGTGTAGGGTTGT
TATAAGCATTGAGTAAGATAAATAATATAAAGCACTTAGAACAGTGCCTGGAACATAAAAACACTTAATA
ATAGCTCATAGCTAACATTTCCTATTTACATTTCTTCTAGAAATAGCCAGTATTTGTTGAGTGCCTACAT
GTTAGTTCCTTTACTAGTTGCTTTACATGTATTATCTTATATTCTGTTTTAAAGTTTCTTCACAGTTACA
GATTTTCATGAAATTTTACTTTTAATAAAAGAGAAGTAAAAGTATAAAGTATTCACTTTTATGTTCACAG
TCTTTTCCTTTAGGCTCATGATGGAGTATCAGAGGCATGAGTGTGTTTAACCTAAGAGCCTTAATGGCTT
GAATCAGAAGCACTTTAGTCCTGTATCTGTTCAGTGTCAGCCTTTCATACATCATTTTAAATCCCATTTG
ACTTTAAGTAAGTCACTTAATCTCTCTACATGTCAATTTCTTCAGCTATAAAATGATGGTATTTCAATAA
ATAAATACATTAATTAAATGATATTATACTGACTAATTGGGCTGTTTTAAGGCTCAATAAGAAAATTTCT
GTGAAAGGTCTCTAGAAAATGTAGGTTCCTATACAAATAAAAGATAACATTGTGCTTATAAAAAAAA
SEQ ID NO: 10
Severse Complement of SEQ ID NO: 9
TTTTTTTTATAAGCACAATGTTATCTTTTATTTGTATAGGAACCTACATTTTCTAGAGACCTTTCACAGAAATTT
TCTTATTGAGCCTTAAAACAGCCCAATTAGTCAGTATAATATCATTTAATTAATGTATTTATTTATTGAAATACC
ATCATTTTATAGCTGAAGAAATTGACATGTAGAGAGATTAAGTGACTTACTTAAAGTCAAATGGGATTTAAAATG
ATGTATGAAAGGCTGACACTGAACAGATACAGGACTAAAGTGCTTCTGATTCAAGCCATTAAGGCTCTTAGGTTA
AACACACTCATGCCTCTGATACTCCATCATGAGCCTAAAGGAAAAGACTGTGAACATAAAAGTGAATACTTTATA
CTTTTACTTCTCTTTTATTAAAAGTAAAATTTCATGAAAATCTGTAACTGTGAAGAAACTTTAAAACAGAATATA
AGATAATACATGTAAAGCAACTAGTAAAGGAACTAACATGTAGGCACTCAACAAATACTGGCTATTTCTAGAAGA
AATGTAAATAGGAAATGTTAGCTATGAGCTATTATTAAGTGTTTTTATGTTCCAGGCACTGTTCTAAGTGCTTTA
TATTATTTATCTTACTCAATGCTTATAACAACCCTACACATTAGGTACTATTACTATTATTGCCATTTTACAGAT
GAGGAAATAGGTGTATAGAGAATTCAGGCACCTTGCCCACGGGTACACAGCATTAATCCAGGGAGTCTGGTTTAA
GGGCACAAACTCTTAAGTACTAAACTCCACTGCTGGATGGAAAAAGATCAGTATAAATATGAATAATTTTGTTCT
ACGCCTAAATAACTTAAGTTCATCTACAGTACAACTTAATATGAAAGGATTCTGTTAGCTTTAATGAGAAGTAAA
ACAAGAAACCAGAATCAAGCAAGGGGCCATGATTTCTTGTCTGGGATGGAAACTCGGTTTCTTTAAATAGCAAAT
GGAATAACACCAAATATATATAGAAATATAATGAGTGAAAAATAACACAAATTTAAGCAACAGTTCAAATACGTA
ATGTCCCTAGAACAATCTAAGTAGACAGTCTGTTATTTTCTTTCTTCCAAATCTTGTCATAGGTGAGCATAAGAT
GGTATCTGCTTCATCCAGCTTTTATGAAAAGAAAAATTCTTACTTGAGAAGAAAGCCTTCATGACAGCTGTCACC
AATATCATCATCATTGAGTACTGTATCAGCTATATCTATTTCTTCAGGAACACTGTGTGATTTCATAGATGAAAG
CAGTTCCATTACAGGAATCACTTCTCCAGTAAGCATTGGAATAATACTCTGACCCTGATCTTCCATTCTCTCTGT
GCCTTCTAAGATAATCTTCTGGACATTTTCTTGTCTTTCCTTATGCATCCATATTCTTCCTTTCCGGATTATATG
TGTTAATCTATCAACACACACTCTATGAAGTGGGAGGTAGAAACTAAGTTCTGTCTGTGGAAGTATAATTGATAG
TCCATATGTGCTGCGATCCCCATTCCAGTTTCCATCAAAGATTAATGAAACAATAATCACTCCCTTTTCAGACAA
GACAAAAAACTTTACATCTATAGCACCACTCTCTGCATTTCGAAGGATTTCTCCATTTAGAGTGTGGTTGGCAAG
AAAAGTTATTTCTCCATCACTGAGAAGTACCTGTTCTGTCTTTGGAGCCCAAATGTGCCTTACTCTAGGACCAAG
AATATTGTCCCAGTAAGCAAAAGTAGCTGCTAATAAAGGTGATTTGCCACTTAAAGCAATCTCTGTCTTGGCAAC
AGCTGGAGATGGCGGTGGGCAAAGAGTCGACATCACTGCATTCCAACTGTCACATTATCCAAATGCTCCGGAGAT
ATCAAGCGTCATCTTTTACGTGGGCGGAACTTGTCGCTGTTTGACGCACCTCTCTTTCCTAGCGGGACACCGTAG
GTTACGT
SEQ ID NO: 11
>NM_018325.5 Homo sapiens C9orf72-SMCR8 complex subunit (C9orf72),
transcript variant 2, mRNA
GGTTGCGGTGCCTGCGCCCGCGGCGGCGGAGGCGCAGGCGGTGGCGAGTGGATATCTCCGGAGCATTTGG
ATAATGTGACAGTTGGAATGCAGTGATGTCGACTCTTTGCCCACCGCCATCTCCAGCTGTTGCCAAGACA
GAGATTGCTTTAAGTGGCAAATCACCTTTATTAGCAGCTACTTTTGCTTACTGGGACAATATTCTTGGTC
CTAGAGTAAGGCACATTTGGGCTCCAAAGACAGAACAGGTACTTCTCAGTGATGGAGAAATAACTTTTCT
TGCCAACCACACTCTAAATGGAGAAATCCTTCGAAATGCAGAGAGTGGTGCTATAGATGTAAAGTTTTTT
GTCTTGTCTGAAAAGGGAGTGATTATTGTTTCATTAATCTTTGATGGAAACTGGAATGGGGATCGCAGCA
CATATGGACTATCAATTATACTTCCACAGACAGAACTTAGTTTCTACCTCCCACTTCATAGAGTGTGTGT
TGATAGATTAACACATATAATCCGGAAAGGAAGAATATGGATGCATAAGGAAAGACAAGAAAATGTCCAG
AAGATTATCTTAGAAGGCACAGAGAGAATGGAAGATCAGGGTCAGAGTATTATTCCAATGCTTACTGGAG
AAGTGATTCCTGTAATGGAACTGCTTTCATCTATGAAATCACACAGTGTTCCTGAAGAAATAGATATAGC
TGATACAGTACTCAATGATGATGATATTGGTGACAGCTGTCATGAAGGCTTTCTTCTCAATGCCATCAGC
TCACACTTGCAAACCTGTGGCTGTTCCGTTGTAGTAGGTAGCAGTGCAGAGAAAGTAAATAAGATAGTCA
GAACATTATGCCTTTTTCTGACTCCAGCAGAGAGAAAATGCTCCAGGTTATGTGAAGCAGAATCATCATT
TAAATATGAGTCAGGGCTCTTTGTACAAGGCCTGCTAAAGGATTCAACTGGAAGCTTTGTGCTGCCTTTC
CGGCAAGTCATGTATGCTCCATATCCCACCACACACATAGATGTGGATGTCAATACTGTGAAGCAGATGC
CACCCTGTCATGAACATATTTATAATCAGCGTAGATACATGAGATCCGAGCTGACAGCCTTCTGGAGAGC
CACTTCAGAAGAAGACATGGCTCAGGATACGATCATCTACACTGACGAAAGCTTTACTCCTGATTTGAAT
ATTTTTCAAGATGTCTTACACAGAGACACTCTAGTGAAAGCCTTCCTGGATCAGGTCTTTCAGCTGAAAC
CTGGCTTATCTCTCAGAAGTACTTTCCTTGCACAGTTTCTACTTGTCCTTCACAGAAAAGCCTTGACACT
AATAAAATATATAGAAGACGATACGCAGAAGGGAAAAAAGCCCTTTAAATCTCTTCGGAACCTGAAGATA
GACCTTGATTTAACAGCAGAGGGCGATCTTAACATAATAATGGCTCTGGCTGAGAAAATTAAACCAGGCC
TACACTCTTTTATCTTTGGAAGACCTTTCTACACTAGTGTGCAAGAACGAGATGTTCTAATGACTTTTTA
AATGTGTAACTTAATAAGCCTATTCCATCACAATCATGATCGCTGGTAAAGTAGCTCAGTGGTGTGGGGA
AACGTTCCCCTGGATCATACTCCAGAATTCTGCTCTCAGCAATTGCAGTTAAGTAAGTTACACTACAGTT
CTCACAAGAGCCTGTGAGGGGATGTCAGGTGCATCATTACATTGGGTGTCTCTTTTCCTAGATTTATGCT
TTTGGGATACAGACCTATGTTTACAATATAATAAATATTATTGCTATCTTTTAAAGATATAATAATAGGA
TGTAAACTTGACCACAACTACTGTTTTTTTGAAATACATGATTCATGGTTTACATGTGTCAAGGTGAAAT
CTGAGTTGGCTTTTACAGATAGTTGACTTTCTATCTTTTGGCATTCTTTGGTGTGTAGAATTACTGTAAT
ACTTCTGCAATCAACTGAAAACTAGAGCCTTTAAATGATTTCAATTCCACAGAAAGAAAGTGAGCTTGAA
CATAGGATGAGCTTTAGAAAGAAAATTGATCAAGCAGATGTTTAATTGGAATTGATTATTAGATCCTACT
TTGTGGATTTAGTCCCTGGGATTCAGTCTGTAGAAATGTCTAATAGTTCTCTATAGTCCTTGTTCCTGGT
GAACCACAGTTAGGGTGTTTTGTTTATTTTATTGTTCTTGCTATTGTTGATATTCTATGTAGTTGAGCTC
TGTAAAAGGAAATTGTATTTTATGTTTTAGTAATTGTTGCCAACTTTTTAAATTAATTTTCATTATTTTT
GAGCCAAATTGAAATGTGCACCTCCTGTGCCTTTTTTCTCCTTAGAAAATCTAATTACTTGGAACAAGTT
CAGATTTCACTGGTCAGTCATTTTCATCTTGTTTTCTTCTTGCTAAGTCTTACCATGTACCTGCTTTGGC
AATCATTGCAACTCTGAGATTATAAAATGCCTTAGAGAATATACTAACTAATAAGATCTTTTTTTCAGAA
ACAGAAAATAGTTCCTTGAGTACTTCCTTCTTGCATTTCTGCCTATGTTTTTGAAGTTGTTGCTGTTTGC
CTGCAATAGGCTATAAGGAATAGCAGGAGAAATTTTACTGAAGTGCTGTTTTCCTAGGTGCTACTTTGGC
AGAGCTAAGTTATCTTTTGTTTTCTTAATGCGTTTGGACCATTTTGCTGGCTATAAAATAACTGATTAAT
ATAATTCTAACACAATGTTGACATTGTAGTTACACAAACACAAATAAATATTTTATTTAAAATTCTGGAA
GTAATATAAAAGGGAAAATATATTTATAAGAAAGGGATAAAGGTAATAGAGCCCTTCTGCCCCCCACCCA
CCAAATTTACACAACAAAATGACATGTTCGAATGTGAAAGGTCATAATAGCTTTCCCATCATGAATCAGA
AAGATGTGGACAGCTTGATGTTTTAGACAACCACTGAACTAGATGACTGTTGTACTGTAGCTCAGTCATT
TAAAAAATATATAAATACTACCTTGTAGTGTCCCATACTGTGTTTTTTACATGGTAGATTCTTATTTAAG
TGCTAACTGGTTATTTTCTTTGGCTGGTTTATTGTACTGTTATACAGAATGTAAGTTGTACAGTGAAATA
AGTTATTAAAGCATGTGTAAACATTGTTATATATCTTTTCTCCTAAATGGAGAATTTTGAATAAAATATA
TTTGAAATTTT
SEQ ID NO: 12
Reverse Complement of SEQ ID NO: 11
AAAATTTCAAATATATTTTATTCAAAATTCTCCATTTAGGAGAAAAGATATATAACAATGTTTACACATGCTTTA
ATAACTTATTTCACTGTACAACTTACATTCTGTATAACAGTACAATAAACCAGCCAAAGAAAATAACCAGTTAGC
ACTTAAATAAGAATCTACCATGTAAAAAACACAGTATGGGACACTACAAGGTAGTATTTATATATTTTTTAAATG
ACTGAGCTACAGTACAACAGTCATCTAGTTCAGTGGTTGTCTAAAACATCAAGCTGTCCACATCTTTCTGATTCA
TGATGGGAAAGCTATTATGACCTTTCACATTCGAACATGTCATTTTGTTGTGTAAATTTGGTGGGTGGGGGGCAG
AAGGGCTCTATTACCTTTATCCCTTTCTTATAAATATATTTTCCCTTTTATATTACTTCCAGAATTTTAAATAAA
ATATTTATTTGTGTTTGTGTAACTACAATGTCAACATTGTGTTAGAATTATATTAATCAGTTATTTTATAGCCAG
CAAAATGGTCCAAACGCATTAAGAAAACAAAAGATAACTTAGCTCTGCCAAAGTAGCACCTAGGAAAACAGCACT
TCAGTAAAATTTCTCCTGCTATTCCTTATAGCCTATTGCAGGCAAACAGCAACAACTTCAAAAACATAGGCAGAA
ATGCAAGAAGGAAGTACTCAAGGAACTATTTTCTGTTTCTGAAAAAAAGATCTTATTAGTTAGTATATTCTCTAA
GGCATTTTATAATCTCAGAGTTGCAATGATTGCCAAAGCAGGTACATGGTAAGACTTAGCAAGAAGAAAACAAGA
TGAAAATGACTGACCAGTGAAATCTGAACTTGTTCCAAGTAATTAGATTTTCTAAGGAGAAAAAAGGCACAGGAG
GTGCACATTTCAATTTGGCTCAAAAATAATGAAAATTAATTTAAAAAGTTGGCAACAATTACTAAAACATAAAAT
ACAATTTCCTTTTACAGAGCTCAACTACATAGAATATCAACAATAGCAAGAACAATAAAATAAACAAAACACCCT
AACTGTGGTTCACCAGGAACAAGGACTATAGAGAACTATTAGACATTTCTACAGACTGAATCCCAGGGACTAAAT
CCACAAAGTAGGATCTAATAATCAATTCCAATTAAACATCTGCTTGATCAATTTTCTTTCTAAAGCTCATCCTAT
GTTCAAGCTCACTTTCTTTCTGTGGAATTGAAATCATTTAAAGGCTCTAGTTTTCAGTTGATTGCAGAAGTATTA
CAGTAATTCTACACACCAAAGAATGCCAAAAGATAGAAAGTCAACTATCTGTAAAAGCCAACTCAGATTTCACCT
TGACACATGTAAACCATGAATCATGTATTTCAAAAAAACAGTAGTTGTGGTCAAGTTTACATCCTATTATTATAT
CTTTAAAAGATAGCAATAATATTTATTATATTGTAAACATAGGTCTGTATCCCAAAAGCATAAATCTAGGAAAAG
AGACACCCAATGTAATGATGCACCTGACATCCCCTCACAGGCTCTTGTGAGAACTGTAGTGTAACTTACTTAACT
GCAATTGCTGAGAGCAGAATTCTGGAGTATGATCCAGGGGAACGTTTCCCCACACCACTGAGCTACTTTACCAGC
GATCATGATTGTGATGGAATAGGCTTATTAAGTTACACATTTAAAAAGTCATTAGAACATCTCGTTCTTGCACAC
TAGTGTAGAAAGGTCTTCCAAAGATAAAAGAGTGTAGGCCTGGTTTAATTTTCTCAGCCAGAGCCATTATTATGT
TAAGATCGCCCTCTGCTGTTAAATCAAGGTCTATCTTCAGGTTCCGAAGAGATTTAAAGGGCTTTTTTCCCTTCT
GCGTATCGTCTTCTATATATTTTATTAGTGTCAAGGCTTTTCTGTGAAGGACAAGTAGAAACTGTGCAAGGAAAG
TACTTCTGAGAGATAAGCCAGGTTTCAGCTGAAAGACCTGATCCAGGAAGGCTTTCACTAGAGTGTCTCTGTGTA
AGACATCTTGAAAAATATTCAAATCAGGAGTAAAGCTTTCGTCAGTGTAGATGATCGTATCCTGAGCCATGTCTT
CTTCTGAAGTGGCTCTCCAGAAGGCTGTCAGCTCGGATCTCATGTATCTACGCTGATTATAAATATGTTCATGAC
AGGGTGGCATCTGCTTCACAGTATTGACATCCACATCTATGTGTGTGGTGGGATATGGAGCATACATGACTTGCC
GGAAAGGCAGCACAAAGCTTCCAGTTGAATCCTTTAGCAGGCCTTGTACAAAGAGCCCTGACTCATATTTAAATG
ATGATTCTGCTTCACATAACCTGGAGCATTTTCTCTCTGCTGGAGTCAGAAAAAGGCATAATGTTCTGACTATCT
TATTTACTTTCTCTGCACTGCTACCTACTACAACGGAACAGCCACAGGTTTGCAAGTGTGAGCTGATGGCATTGA
GAAGAAAGCCTTCATGACAGCTGTCACCAATATCATCATCATTGAGTACTGTATCAGCTATATCTATTTCTTCAG
GAACACTGTGTGATTTCATAGATGAAAGCAGTTCCATTACAGGAATCACTTCTCCAGTAAGCATTGGAATAATAC
TCTGACCCTGATCTTCCATTCTCTCTGTGCCTTCTAAGATAATCTTCTGGACATTTTCTTGTCTTTCCTTATGCA
TCCATATTCTTCCTTTCCGGATTATATGTGTTAATCTATCAACACACACTCTATGAAGTGGGAGGTAGAAACTAA
GTTCTGTCTGTGGAAGTATAATTGATAGTCCATATGTGCTGCGATCCCCATTCCAGTTTCCATCAAAGATTAATG
AAACAATAATCACTCCCTTTTCAGACAAGACAAAAAACTTTACATCTATAGCACCACTCTCTGCATTTCGAAGGA
TTTCTCCATTTAGAGTGTGGTTGGCAAGAAAAGTTATTTCTCCATCACTGAGAAGTACCTGTTCTGTCTTTGGAG
CCCAAATGTGCCTTACTCTAGGACCAAGAATATTGTCCCAGTAAGCAAAAGTAGCTGCTAATAAAGGTGATTTGC
CACTTAAAGCAATCTCTGTCTTGGCAACAGCTGGAGATGGCGGTGGGCAAAGAGTCGACATCACTGCATTCCAAC
TGTCACATTATCCAAATGCTCCGGAGATATCCACTCGCCACCGCCTGCGCCTCCGCCGCCGCGGGCGCAGGCACC
GCAACC
SEQ ID NO: 13
>NC_000009.12: c27573866-27546546 Homo sapiens chromosome 9, GRCh38.p13
Primary Assembly; portion of human chromosome 9 harboring the C9orf72 gene
(nucleotides 27546546 . . . 27573866 of the assembly of chromosome 9)
ACGTAACCTACGGTGTCCCGCTAGGAAAGAGAGGTGCGTCAAACAGCGACAAGTTCCGCCCACGTAAAAGATGAC
GCTTGGTGTGTCAGCCGTCCCTGCTGCCCGGTTGCTTCTCTTTTGGGGGCGGGGTCTAGCAAGAGCAGGTGTGGG
TTTAGGAGGTGTGTGTTTTTGTTTTTCCCACCCTCTCTCCCCACTACTTGCTCTCACAGTACTCGCTGAGGGTGA
ACAAGAAAAGACCTGATAAAGATTAACCAGAAGAAAACAAGGAGGGAAACAACCGCAGCCTGTAGCAAGCTCTGG
AACTCAGGAGTCGCGCGCTAGGGGCCGGGGCCGGGGCCGGGGCGTGGTCGGGGGGGGCCCGGGGGCGGGCCCGGG
GCGGGGCTGCGGTTGCGGTGCCTGCGCCCGCGGCGGCGGAGGCGCAGGCGGTGGCGAGTGGGTGAGTGAGGAGGC
GGCATCCTGGCGGGTGGCTGTTTGGGGTTCGGCTGCCGGGAAGAGGCGCGGGTAGAAGCGGGGGCTCTCCTCAGA
GCTCGACGCATTTTTACTTTCCCTCTCATTTCTCTGACCGAAGCTGGGTGTCGGGCTTTCGCCTCTAGCGACTGG
TGGAATTGCCTGCATCCGGGCCCCGGGCTTCCCGGCGGCGGCGGCGGCGGCGGCGGCGCAGGGACAAGGGATGGG
GATCTGGCCTCTTCCTTGCTTTCCCGCCCTCAGTACCCGAGCTGTCTCCTTCCCGGGGACCCGCTGGGAGCGCTG
CCGCTGCGGGCTCGAGAAAAGGGAGCCTCGGGTACTGAGAGGCCTCGCCTGGGGGAAGGCCGGAGGGTGGGCGGC
GCGCGGCTTCTGCGGACCAAGTCGGGGTTCGCTAGGAACCCGAGACGGTCCCTGCCGGCGAGGAGATCATGCGGG
ATGAGATGGGGGTGTGGAGACGCCTGCACAATTTCAGCCCAAGCTTCTAGAGAGTGGTGATGACTTGCATATGAG
GGCAGCAATGCAAGTCGGTGTGCTCCCCATTCTGTGGGACATGACCTGGTTGCTTCACAGCTCCGAGATGACACA
GACTTGCTTAAAGGAAGTGACTATTGTGACTTGGGCATCACTTGACTGATGGTAATCAGTTGTCTAAAGAAGTGC
ACAGATTACATGTCCGTGTGCTCATTGGGTCTATCTGGCCGCGTTGAACACCACCAGGCTTTGTATTCAGAAACA
GGAGGGAGGTCCTGCACTTTCCCAGGAGGGGTGGCCCTTTCAGATGCAATCGAGATTGTTAGGCTCTGGGAGAGT
AGTTGCCTGGTTGTGGCAGTTGGTAAATTTCTATTCAAACAGTTGCCATGCACCAGTTGTTCACAACAAGGGTAC
GTAATCTGTCTGGCATTACTTCTACTTTTGTACAAAGGATCAAAAAAAAAAAAGATACTGTTAAGATATGATTTT
TCTCAGACTTTGGGAAACTTTTAACATAATCTGTGAATATCACAGAAACAAGACTATCATATAGGGGATATTAAT
AACCTGGAGTCAGAATACTTGAAATACGGTGTCATTTGACACGGGCATTGTTGTCACCACCTCTGCCAAGGCCTG
CCACTTTAGGAAAACCCTGAATCAGTTGGAAACTGCTACATGCTGATAGTACATCTGAAACAAGAACGAGAGTAA
TTACCACATTCCAGATTGTTCACTAAGCCAGCATTTACCTGCTCCAGGAAAAAATTACAAGCACCTTATGAAGTT
GATAAAATATTTTGTTTGGCTATGTTGGCACTCCACAATTTGCTTTCAGAGAAACAAAGTAAACCAAGGAGGACT
TCTGTTTTTCAAGTCTGCCCTCGGGTTCTATTCTACGTTAATTAGATAGTTCCCAGGAGGACTAGGTTAGCCTAC
CTATTGTCTGAGAAACTTGGAACTGTGAGAAATGGCCAGATAGTGATATGAACTTCACCTTCCAGTCTTCCCTGA
TGTTGAAGATTGAGAAAGTGTTGTGAACTTTCTGGTACTGTAAACAGTTCACTGTCCTTGAAGTGGTCCTGGGCA
GCTCCTGTTGTGGAAAGTGGACGGTTTAGGATCCTGCTTCTCTTTGGGCTGGGAGAAAATAAACAGCATGGTTAC
AAGTATTGAGAGCCAGGTTGGAGAAGGTGGCTTACACCTGTAATGCCAGAGCTTTGGGAGGCGGAGGCAAGAGGA
TCACTTGAAGCCAGGAGTTCAAGCTCAACCTGGGCAACGTAGACCCTGTCTCTACAAAAAATTAAAAACTTAGCC
GGGCGTGGTGATGTGCACCTGTAGTCCTAGCTACTTGGGAGGCTGAGGCAGGAGGGTCATTTGAGCCCAAGAGTT
TGAAGTTACCGAGAGCTATGATCCTGCCAGTGCATTCCAGCCTGGATGACAAAACGAGACCCTGTCTCTAAAAAA
CAAGAAGTGAGGGCTTTATGATTGTAGAATTTTCACTACAATAGCAGTGGACCAACCACCTTTCTAAATACCAAT
CAGGGAAGAGATGGTTGATTTTTTAACAGACGTTTAAAGAAAAAGCAAAACCTCAAACTTAGCACTCTACTAACA
GTTTTAGCAGATGTTAATTAATGTAATCATGTCTGCATGTATGGGATTATTTCCAGAAAGTGTATTGGGAAACCT
CTCATGAACCCTGTGAGCAAGCCACCGTCTCACTCAATTTGAATCTTGGCTTCCCTCAAAAGACTGGCTAATGTT
TGGTAACTCTCTGGAGTAGACAGCACTACATGTACGTAAGATAGGTACATAAACAACTATTGGTTTTGAGCTGAT
TTTTTTCAGCTGCATTTGCATGTATGGATTTTTCTCACCAAAGACGATGACTTCAAGTATTAGTAAAATAATTGT
ACAGCTCTCCTGATTATACTTCTCTGTGACATTTCATTTCCCAGGCTATTTCTTTTGGTAGGATTTAAAACTAAG
CAATTCAGTATGATCTTTGTCCTTCATTTTCTTTCTTATTCTTTTTGTTTGTTTGTTTGTTTGTTTTTTTCTTGA
GGCAGAGTCTCTCTCTGTCGCCCAGGCTGGAGTGCAGTGGCGCCATCTCAGCTCATTGCAACCTCTGCCACCTCC
GGGTTCAAGAGATTCTCCTGCCTCAGCCTCCCGAGTAGCTGGGATTACAGGTGTCCACCACCACACCCGGCTAAT
TTTTTGTATTTTTAGTAGAGGTGGGGTTTCACCATGTTGGCCAGGCTGGTCTTGAGCTCCTGACCTCAGGTGATC
CACCTGCCTCGGCCTACCAAAGAGCTGGGATAACAGGTGTGACCCACCATGCCCGGCCCATTTTTTTTTTCTTAT
TCTGTTAGGAGTGAGAGTGTAACTAGCAGTATAATAGTTCAATTTTCACAACGTGGTAAAAGTTTCCCTATAATT
CAATCAGATTTTGCTCCAGGGTTCAGTTCTGTTTTAGGAAATACTTTTATTTTCAGTTTAATGATGAAATATTAG
AGTTGTAATATTGCCTTTATGATTATCCACCTTTTTAACCTAAAAGAATGAAAGAAAAATATGTTTGCAATATAA
TTTTATGGTTGTATGTTAACTTAATTCATTATGTTGGCCTCCAGTTTGCTGTTGTTAGTTATGACAGCAGTAGTG
TCATTACCATTTCAATTCAGATTACATTCCTATATTTGATCATTGTAAACTGACTGCTTACATTGTATTAAAAAC
AGTGGATATTTTAAAGAAGCTGTACGGCTTATATCTAGTGCTGTCTCTTAAGACTATTAAATTGATACAACATAT
TTAAAAGTAAATATTACCTAAATGAATTTTTGAAATTACAAATACACGTGTTAAAACTGTCGTTGTGTTCAACCA
TTTCTGTACATACTTAGAGTTAACTGTTTTGCCAGGCTCTGTATGCCTACTCATAATATGATAAAAGCACTCATC
TAATGCTCTGTAAATAGAAGTCAGTGCTTTCCATCAGACTGAACTCTCTTGACAAGATGTGGATGAAATTCTTTA
AGTAAAATTGTTTACTTTGTCATACATTTACAGATCAAATGTTAGCTCCCAAAGCAATCATATGGCAAAGATAGG
TATATCATAGTTTGCCTATTAGCTGCTTTGTATTGCTATTATTATAAATAGACTTCACAGTTTTAGACTTGCTTA
GGTGAAATTGCAATTCTTTTTACTTTCAGTCTTAGATAACAAGTCTTCAATTATAGTACAATCACACATTGCTTA
GGAATGCATCATTAGGCGATTTTGTCATTATGCAAACATCATAGAGTGTACTTACACAAACCTAGATAGTATAGC
CTTTATGTACCTAGGCCGTATGGTATAGTCTGTTGCTCCTAGGCCACAAACCTGTACAACTGTTACTGTACTGAA
TACTATAGACAGTTGTAACACAGTGGTAAATATTTATCTAAATATATGCAAACAGAGAAAAGGTACAGTAAAAGT
ATGGTATAAAAGATAATGGTATACCTGTGTAGGCCACTTACCACGAATGGAGCTTGCAGGACTAGAAGTTGCTCT
GGGTGAGTCAGTGAGTGAGTGGTGAATTAATGTGAAGGCCTAGAACACTGTACACCACTGTAGACTATAAACACA
GTACGCTGAAGCTACACCAAATTTATCTTAACAGTTTTTCTTCAATAAAAAATTATAACTTTTTAACTTTGTAAA
CTTTTTAATTTTTTAACTTTTAAAATACTTAGCTTGAAACACAAATACATTGTATAGCTATACAAAAATATTTTT
TCTTTGTATCCTTATTCTAGAAGCTTTTTTCTATTTTCTATTTTAAATTTTTTTTTTTACTTGTTAGTCGTTTTT
GTTAAAAACTAAAACACACACACTTTCACCTAGGCATAGACAGGATTAGGATCATCAGTATCACTCCCTTCCACC
TCACTGCCTTCCACCTCCACATCTTGTCCCACTGGAAGGTTTTTAGGGGCAATAACACACATGTAGCTGTCACCT
ATGATAACAGTGCTTTCTGTTGAATACCTCCTGAAGGACTTGCCTGAGGCTGTTTTACATTTAACTTAAAAAAAA
AAAAAGTAGAAGGAGTGCACTCTAAAATAACAATAAAAGGCATAGTATAGTGAATACATAAACCAGCAATGTAGT
AGTTTATTATCAAGTGTTGTACACTGTAATAATTGTATGTGCTATACTTTAAATAACTTGCAAAATAGTACTAAG
ACCTTATGATGGTTACAGTGTCACTAAGGCAATAGCATATTTTCAGGTCCATTGTAATCTAATGGGACTACCATC
ATATATGCAGTCTACCATTGACTGAAACGTTACATGGCACATAACTGTATTTGCAAGAATGATTTGTTTTACATT
AATATCACATAGGATGTACCTTTTTAGAGTGGTATGTTTATGTGGATTAAGATGTACAAGTTGAGCAAGGGGACC
AAGAGCCCTGGGTTCTGTCTTGGATGTGAGCGTTTATGTTCTTCTCCTCATGTCTGTTTTCTCATTAAATTCAAA
GGCTTGAACGGGCCCTATTTAGCCCTTCTGTTTTCTACGTGTTCTAAATAACTAAAGCTTTTAAATTCTAGCCAT
TTAGTGTAGAACTCTCTTTGCAGTGATGAAATGCTGTATTGGTTTCTTGGCTAGCATATTAAATATTTTTATCTT
TGTCTTGATACTTCAATGTCGTTTTAAACATCAGGATCGGGCTTCAGTATTCTCATAACCAGAGAGTTCACTGAG
GATACAGGACTGTTTGCCCATTTTTTGTTATGGCTCCAGACTTGTGGTATTTCCATGTCTTTTTTTTTTTTTTTT
TTTTTGACCTTTTAGCGGCTTTAAAGTATTTCTGTTGTTAGGTGTTGTATTACTTTTCTAAGATTACTTAACAAA
GCACCACAAACTGAGTGGCTTTAAACAACAGCAATTTATTCTCTCACAATTCTAGAAGCTAGAAGTCCGAAATCA
AAGTGTTGACAGGGGCATGATCTTCAAGAGAGAAGACTCTTTCCTTGCCTCTTCCTGGCTTCTGGTGGTTACCAG
CAATCCTGAGTGTTCCTTTCTTGCCTTGTAGTTTCAACAATCCAGTATCTGCCTTTTGTCTTCACATGGCTGTCT
ACCATTTGTCTCTGTGTCTCCAAATCTCTCTCCTTATAAACACAGCAGTTATTGGATTAGGCCCCACTCTAATCC
AGTATGACCCCATTTTAACATGATTACACTTATTTCTAGATAAGGTCACATTCACGTACACCAAGGGTTAGGAAT
TGAACATATCTTTTTGGGGGACACAATTCAACCCACAAGTGTCAGTCTCTAGCTGAGCCTTTCCCTTCCTGTTTT
TCTCCTTTTTAGTTGCTATGGGTTAGGGGCCAAATCTCCAGTCATACTAGAATTGCACATGGACTGGATATTTGG
GAATACTGCGGGTCTATTCTATGAGCTTTAGTATGTAACATTTAATATCAGTGTAAAGAAGCCCTTTTTTAAGTT
ATTTCTTTGAATTTCTAAATGTATGCCCTGAATATAAGTAACAAGTTACCATGTCTTGTAAAATGATCATATCAA
CAAACATTTAATGTGCACCTACTGTGCTAGTTGAATGTCTTTATCCTGATAGGAGATAACAGGATTCCACATCTT
TGACTTAAGAGGACAAACCAAATATGTCTAAATCATTTGGGGTTTTGATGGATATCTTTAAATTGCTGAACCTAA
TCATTGGTTTCATATGTCATTGTTTAGATATCTCCGGAGCATTTGGATAATGTGACAGTTGGAATGCAGTGATGT
CGACTCTTTGCCCACCGCCATCTCCAGCTGTTGCCAAGACAGAGATTGCTTTAAGTGGCAAATCACCTTTATTAG
CAGCTACTTTTGCTTACTGGGACAATATTCTTGGTCCTAGAGTAAGGCACATTTGGGCTCCAAAGACAGAACAGG
TACTTCTCAGTGATGGAGAAATAACTTTTCTTGCCAACCACACTCTAAATGGAGAAATCCTTCGAAATGCAGAGA
GTGGTGCTATAGATGTAAAGTTTTTTGTCTTGTCTGAAAAGGGAGTGATTATTGTTTCATTAATCTTTGATGGAA
ACTGGAATGGGGATCGCAGCACATATGGACTATCAATTATACTTCCACAGACAGAACTTAGTTTCTACCTCCCAC
TTCATAGAGTGTGTGTTGATAGATTAACACATATAATCCGGAAAGGAAGAATATGGATGCATAAGGTAAGTGATT
TTTCAGCTTATTAATCATGTTAACCTATCTGTTGAAAGCTTATTTTCTGGTACATATAAATCTTATTTTTTTAAT
TATATGCAGTGAACATCAAACAATAAATGTTATTTATTTTGCATTTACCCTATTAGATACAAATACATCTGGTCT
GATACCTGTCATCTTCATATTAACTGTGGAAGGTACGAAATGGTAGCTCCACATTATAGATGAAAAGCTAAAGCT
TAGACAAATAAAGAAACTTTTAGACCCTGGATTCTTCTTGGGAGCCTTTGACTCTAATACCTTTTGTTTCCCTTT
CATTGCACAATTCTGTCTTTTGCTTACTACTATGTGTAAGTATAACAGTTCAAAGTAATAGTTTCATAAGCTGTT
GGTCATGTAGCCTTTGGTCTCTTTAACCTCTTTGCCAAGTTCCCAGGTTCATAAAATGAGGAGGTTGAATGGAAT
GGTTCCCAAGAGAATTCCTTTTAATCTTACAGAAATTATTGTTTTCCTAAATCCTGTAGTTGAATATATAATGCT
ATTTACATTTCAGTATAGTTTTGATGTATCTAAAGAACACATTGAATTCTCCTTCCTGTGTTCCAGTTTGATACT
AACCTGAAAGTCCATTAAGCATTACCAGTTTTAAAAGGCTTTTGCCCAATAGTAAGGAAAAATAATATCTTTTAA
AAGAATAATTTTTTACTATGTTTGCAGGCTTACTTCCTTTTTTCTCACATTATGAAACTCTTAAAATCAGGAGAA
TCTTTTAAACAACATCATAATGTTTAATTTGAAAAGTGCAAGTCATTCTTTTCCTTTTTGAAACTATGCAGATGT
TACATTGACTGTTTTCTGTGAAGTTATCTTTTTTTCACTGCAGAATAAAGGTTGTTTTGATTTTATTTTGTATTG
TTTATGAGAACATGCATTTGTTGGGTTAATTTCCTACCCCTGCCCCCATTTTTTCCCTAAAGTAGAAAGTATTTT
TCTTGTGAACTAAATTACTACACAAGAACATGTCTATTGAAAAATAAGCAAGTATCAAAATGTTGTGGGTTGTTT
TTTTAAATAAATTTTCTCTTGCTCAGGAAAGACAAGAAAATGTCCAGAAGATTATCTTAGAAGGCACAGAGAGAA
TGGAAGATCAGGTATATGCAAATTGCATACTGTCAAATGTTTTTCTCACAGCATGTATCTGTATAAGGTTGATGG
CTACATTTGTCAAGGCCTTGGAGACATACGAATAAGCCTTTAATGGAGCTTTTATGGAGGTGTACAGAATAAACT
GGAGGAAGATTTCCATATCTTAAACCCAAAGAGTTAAATCAGTAAACAAAGGAAAATAGTAATTGCATCTACAAA
TTAATATTTGCTCCCTTTTTTTTTCTGTTTGCCCAGAATAAATTTTGGATAACTTGTTCATAGTAAAAATAAAAA
AAATTGTCTCTGATATGTTCTTTAAGGTACTACTTCTCGAACCTTTCCCTAGAAGTAGCTGTAACAGAAGGAGAG
CATATGTACCCCTGAGGTATCTGTCTGGGGTGTAGGCCCAGGTCCACACAATATTTCTTCTAAGTCTTATGTTGT
ATCGTTAAGACTCATGCAATTTACATTTTATTCCATAACTATTTTAGTATTAAAATTTGTCAGTGATATTTCTTA
CCCTCTCCTCTAGGAAAATGTGCCATGTTTATCCCTTGGCTTTGAATGCCCCTCAGGAACAGACACTAAGAGTTT
GAGAAGCATGGTTACAAGGGTGTGGCTTCCCCTGCGGAAACTAAGTACAGACTATTTCACTGTAAAGCAGAGAAG
TTCTTTTGAAGGAGAATCTCCAGTGAAGAAAGAGTTCTTCACTTTTACTTCCATTTCCTCTTGTGGGTGACCCTC
AATGCTCCTTGTAAAACTCCAATATTTTAAACATGGCTGTTTTGCCTTTCTTTGCTTCTTTTTAGCATGAATGAG
ACAGATGATACTTTAAAAAAGTAATTAAAAAAAAAAACTTGTGAAAATACATGGCCATAATACAGAACCCAATAC
AATGATCTCCTTTACCAAATTGTTATGTTTGTACTTTTGTAGATAGCTTTCCAATTCAGAGACAGTTATTCTGTG
TAAAGGTCTGACTTAACAAGAAAAGATTTCCCTTTACCCAAAGAATCCCAGTCCTTATTTGCTGGTCAATAAGCA
GGGTCCCCAGGAATGGGGTAACTTTCAGCACCCTCTAACCCACTAGTTATTAGTAGACTAATTAAGTAAACTTAT
CGCAAGTTGAGGAAACTTAGAACCAACTAAAATTCTGCTTTTACTGGGATTTTGTTTTTTCAAACCAGAAACCTT
TACTTAAGTTGACTACTATTAATGAATTTTGGTCTCTCTTTTAAGTGCTCTTCTTAAAAATGTTATCTTACTGCT
GAGAAGTTCAAGTTTGGGAAGTACAAGGAGGAATAGAAACTTAAGAGATTTTCTTTTAGAGCCTCTTCTGTATTT
AGCCCTGTAGGATTTTTTTTTTTTTTTTTTTTTTTGGTGTTGTTGAGCTTCAGTGAGGCTATTCATTCACTTATA
CTGATAATGTCTGAGATACTGTGAATGAAATACTATGTATGCTTAAACCTAAGAGGAAATATTTTCCCAAAATTA
TTCTTCCCGAAAAGGAGGAGTTGCCTTTTGATTGAGTTCTTGCAAATCTCACAACGACTTTATTTTGAACAATAC
TGTTTGGGGATGATGCATTAGTTTGAAACAACTTCAGTTGTAGCTGTCATCTGATAAAATTGCTTCACAGGGAAG
GAAATTTAACACGGATCTAGTCATTATTCTTGTTAGATTGAATGTGTGAATTGTAATTGTAAACAGGCATGATAA
TTATTACTTTAAAAACTAAAAACAGTGAATAGTTAGTTGTGGAGGTTACTAAAGGATGGTTTTTTTTTAAATAAA
ACTTTCAGCATTATGCAAATGGGCATATGGCTTAGGATAAAACTTCCAGAAGTAGCATCACATTTAAATTCTCAA
GCAACTTAATAATATGGGGCTCTGAAAAACTGGTTAAGGTTACTCCAAAAATGGCCCTGGGTCTGACAAAGATTC
TAACTTAAAGATGCTTATGAAGACTTTGAGTAAAATCATTTCATAAAATAAGTGAGGAAAAACAACTAGTATTAA
ATTCATCTTAAATAATGTATGATTTAAAAAATATGTTTAGCTAAAAATGCATAGTCATTTGACAATTTCATTTAT
ATCTCAAAAAATTTACTTAACCAAGTTGGTCACAAAACTGATGAGACTGGTGGTGGTAGTGAATAAATGAGGGAC
CATCCATATTTGAGACACTTTACATTTGTGATGTGTTATACTGAATTTTCAGTTTGATTCTATAGACTACAAATT
TCAAAATTACAATTTCAAGATGTAATAAGTAGTAATATCTTGAAATAGCTCTAAAGGGAATTTTTCTGTTTTATT
GATTCTTAAAATATATGTGCTGATTTTGATTTGCATTTGGGTAGATTATACTTTTATGAGTATGGAGGTTAGGTA
TTGATTCAAGTTTTCCTTACCTATTTGGTAAGGATTTCAAAGTCTTTTTGTGCTTGGTTTTCCTCATTTTTAAAT
ATGAAATATATTGATGACCTTTAACAAATTTTTTTTATCTCAAATTTTAAAGGAGATCTTTTCTAAAAGAGGCAT
GATGACTTAATCATTGCATGTAACAGTAAACGATAAACCAATGATTCCATACTCTCTAAAGAATAAAAGTGAGCT
TTAGGGCCGGGCATGGTCAGAAATTTGACACCAACCTGGCCAACATGGCGAAACCCCGTCTCTACTAAAAATACA
AAAATCAGCCGGGCATGGTGGCGGCACCTATAGTCCCAGCTACTTGGGAGGATGAGACAGGAGAGTCACTTGAAC
CTGGGAGGAGAGGTTGCAGTGAGCTGAGATCACGCCATTGCACTCCAGCCTGAGCAATGAAAGCAAAACTCCATC
TCAAAAAAAAAAAAAGAAAAGAAAGAATAAAAGTGAGCTTTGGATTGCATATAAATCCTTTAGACATGTAGTAGA
CTTGTTTGATACTGTGTTTGAACAAATTACGAAGTATTTTCATCAAAGAATGTTATTGTTTGATGTTATTTTTAT
TTTTTATTGCCCAGCTTCTCTCATATTACGTGATTTTCTTCACTTCATGTCACTTTATTGTGCAGGGTCAGAGTA
TTATTCCAATGCTTACTGGAGAAGTGATTCCTGTAATGGAACTGCTTTCATCTATGAAATCACACAGTGTTCCTG
AAGAAATAGATGTAAGTTTAAATGAGAGCAATTATACACTTTATGAGTTTTTTGGGGTTATAGTATTATTATGTA
TATTATTAATATTCTAATTTTAATAGTAAGGACTTTGTCATACATACTATTCACATACAGTATTAGCCACTTTAG
CAAATAAGCACACACAAAATCCTGGATTTTATGGCAAAACAGAGGCATTTTTGATCAGTGATGACAAAATTAAAT
TCATTTTGTTTATTTCATTACTTTTATAATTCCTAAAAGTGGGAGGATCCCAGCTCTTATAGGAGCAATTAATAT
TTAATGTAGTGTCTTTTGAAACAAAACTGTGTGCCAAAGTAGTAACCATTAATGGAAGTTTACTTGTAGTCACAA
ATTTAGTTTCCTTAATCATTTGTTGAGGACGTTTTGAATCACACACTATGAGTGTTAAGAGATACCTTTAGGAAA
CTATTCTTGTTGTTTTCTGATTTTGTCATTTAGGTTAGTCTCCTGATTCTGACAGCTCAGAAGAGGAAGTTGTTC
TTGTAAAAATTGTTTAACCTGCTTGACCAGCTTTCACATTTGTTCTTCTGAAGTTTATGGTAGTGCACAGAGATT
GTTTTTTGGGGAGTCTTGATTCTCGGAAATGAAGGCAGTGTGTTATATTGAATCCAGACTTCCGAAAACTTGTAT
ATTAAAAGTGTTATTTCAACACTATGTTACAGCCAGACTAATTTTTTTATTTTTTGATGCATTTTAGATAGCTGA
TACAGTACTCAATGATGATGATATTGGTGACAGCTGTCATGAAGGCTTTCTTCTCAAGTAAGAATTTTTCTTTTC
ATAAAAGCTGGATGAAGCAGATACCATCTTATGCTCACCTATGACAAGATTTGGAAGAAAGAAAATAACAGACTG
TCTACTTAGATTGTTCTAGGGACATTACGTATTTGAACTGTTGCTTAAATTTGTGTTATTTTTCACTCATTATAT
TTCTATATATATTTGGTGTTATTCCATTTGCTATTTAAAGAAACCGAGTTTCCATCCCAGACAAGAAATCATGGC
CCCTTGCTTGATTCTGGTTTCTTGTTTTACTTCTCATTAAAGCTAACAGAATCCTTTCATATTAAGTTGTACTGT
AGATGAACTTAAGTTATTTAGGCGTAGAACAAAATTATTCATATTTATACTGATCTTTTTCCATCCAGCAGTGGA
GTTTAGTACTTAAGAGTTTGTGCCCTTAAACCAGACTCCCTGGATTAATGCTGTGTACCCGTGGGCAAGGTGCCT
GAATTCTCTATACACCTATTTCCTCATCTGTAAAATGGCAATAATAGTAATAGTACCTAATGTGTAGGGTTGTTA
TAAGCATTGAGTAAGATAAATAATATAAAGCACTTAGAACAGTGCCTGGAACATAAAAACACTTAATAATAGCTC
ATAGCTAACATTTCCTATTTACATTTCTTCTAGAAATAGCCAGTATTTGTTGAGTGCCTACATGTTAGTTCCTTT
ACTAGTTGCTTTACATGTATTATCTTATATTCTGTTTTAAAGTTTCTTCACAGTTACAGATTTTCATGAAATTTT
ACTTTTAATAAAAGAGAAGTAAAAGTATAAAGTATTCACTTTTATGTTCACAGTCTTTTCCTTTAGGCTCATGAT
GGAGTATCAGAGGCATGAGTGTGTTTAACCTAAGAGCCTTAATGGCTTGAATCAGAAGCACTTTAGTCCTGTATC
TGTTCAGTGTCAGCCTTTCATACATCATTTTAAATCCCATTTGACTTTAAGTAAGTCACTTAATCTCTCTACATG
TCAATTTCTTCAGCTATAAAATGATGGTATTTCAATAAATAAATACATTAATTAAATGATATTATACTGACTAAT
TGGGCTGTTTTAAGGCTCAATAAGAAAATTTCTGTGAAAGGTCTCTAGAAAATGTAGGTTCCTATACAAATAAAA
GATAACATTGTGCTTATAGCTTCGGTGTTTATCATATAAAGCTATTCTGAGTTATTTGAAGAGCTCACCTACTTT
TTTTTGTTTTTAGTTTGTTAAATTGTTTTATAGGCAATGTTTTTAATCTGTTTTCTTTAACTTACAGTGCCATCA
GCTCACACTTGCAAACCTGTGGCTGTTCCGTTGTAGTAGGTAGCAGTGCAGAGAAAGTAAATAAGGTAGTTTATT
TTATAATCTAGCAAATGATTTGACTCTTTAAGACTGATGATATATCATGGATTGTCATTTAAATGGTAGGTTGCA
ATTAAAATGATCTAGTAGTATAAGGAGGCAATGTAATCTCATCAAATTGCTAAGACACCTTGTGGCAACAGTGAG
TTTGAAATAAACTGAGTAAGAATCATTTATCAGTTTATTTTGATAGCTCGGAAATACCAGTGTCAGTAGTGTATA
AATGGTTTTGAGAATATATTAAAATCAGATATATAAAAAAAATTACTCTTCTATTTCCCAATGTTATCTTTAACA
AATCTGAAGATAGTCATGTACTTTTGGTAGTAGTTCCAAAGAAATGTTATTTGTTTATTCATCTTGATTTCATTG
TCTTCGCTTTCCTTCTAAATCTGTCCCTTCTAGGGAGCTATTGGGATTAAGTGGTCATTGATTATTATACTTTAT
TCAGTAATGTTTCTGACCCTTTCCTTCAGTGCTACTTGAGTTAATTAAGGATTAATGAACAGTTACATTTCCAAG
CATTAGCTAATAAACTAAAGGATTTTGCACTTTTCTTCACTGACCATTAGTTAGAAAGAGTTCAGAGATAAGTAT
GTGTATCTTTCAATTTCAGCAAACCTAATTTTTTAAAAAAAGTTTTACATAGGAAATATGTTGGAAATGATACTT
TACAAAGATATTCATAATTTTTTTTTGTAATCAGCTACTTTGTATATTTACATGAGCCTTAATTTATATTTCTCA
TATAACCATTTATGAGAGCTTAGTATACCTGTGTCATTATATTGCATCTACGAACTAGTGACCTTATTCCTTCTG
TTACCTCAAACAGGTGGCTTTCCATCTGTGATCTCCAAAGCCTTAGGTTGCACAGAGTGACTGCCGAGCTGCTTT
ATGAAGGGAGAAAGGCTCCATAGTTGGAGTGTTTTTTTTTTTTTTTTTAAACATTTTTCCCATCCTCCATCCTCT
TGAGGGAGAATAGCTTACCTTTTATCTTGTTTTAATTTGAGAAAGAAGTTGCCACCACTCTAGGTTGAAAACCAC
TCCTTTAACATAATAACTGTGGATATGGTTTGAATTTCAAGATAGTTACATGCCTTTTTATTTTTCCTAATAGAG
CTGTAGGTCAAATATTATTAGAATCAGATTTCTAAATCCCACCCAATGACCTGCTTATTTTAAATCAAATTCAAT
AATTAATTCTCTTCTTTTTGGAGGATCTGGACATTCTTTGATATTTCTTACAACGAATTTCATGTGTAGACCCAC
TAAACAGAAGCTATAAAAGTTGCATGGTCAAATAAGTCTGAGAAAGTCTGCAGATGATATAATTCACCTGAAGAG
TCACAGTATGTAGCCAAATGTTAAAGGTTTTGAGATGCCATACAGTAAATTTACCAAGCATTTTCTAAATTTATT
TGACCACAGAATCCCTATTTTAAGCAACAACTGTTACATCCCATGGATTCCAGGTGACTAAAGAATACTTATTTC
TTAGGATATGTTTTATTGATAATAACAATTAAAATTTCAGATATCTTTCATAAGCAAATCAGTGGTCTTTTTACT
TCATGTTTTAATGCTAAAATATTTTCTTTTATAGATAGTCAGAACATTATGCCTTTTTCTGACTCCAGCAGAGAG
AAAATGCTCCAGGTTATGTGAAGCAGAATCATCATTTAAATATGAGTCAGGGCTCTTTGTACAAGGCCTGCTAAA
GGTATAGTTTCTAGTTATCACAAGTGAAACCACTTTTCTAAAATCATTTTTGAGACTCTTTATAGACAAATCTTA
AATATTAGCATTTAATGTATCTCATATTGACATGCCCAGAGACTGACTTCCTTTACACAGTTCTGCACATAGACT
ATATGTCTTATGGATTTATAGTTAGTATCATCAGTGAAACACCATAGAATACCCTTTGTGTTCCAGGTGGGTCCC
TGTTCCTACATGTCTAGCCTCAGGACTTTTTTTTTTTTAACACATGCTTAAATCAGGTTGCACATCAAAAATAAG
ATCATTTCTTTTTAACTAAATAGATTTGAATTTTATTGAAAAAAAATTTTAAACATCTTTAAGAAGCTTATAGGA
TTTAAGCAATTCCTATGTATGTGTACTAAAATATATATATTTCTATATATAATATATATTAGAAAAAAATTGTAT
TTTTCTTTTATTTGAGTCTACTGTCAAGGAGCAAAACAGAGAAATGTAAATTAGCAATTATTTATAATACTTAAA
GGGAAGAAAGTTGTTCACCTTGTTGAATCTATTATTGTTATTTCAATTATAGTCCCAAGACGTGAAGAAATAGCT
TTCCTAATGGTTATGTGATTGTCTCATAGTGACTACTTTCTTGAGGATGTAGCCACGGCAAAATGAAATAAAAAA
ATTTAAAAATTGTTGCAAATACAAGTTATATTAGGCTTTTGTGCATTTTCAATAATGTGCTGCTATGAACTCAGA
ATGATAGTATTTAAATATAGAAACTAGTTAAAGGAAACGTAGTTTCTATTTGAGTTATACATATCTGTAAATTAG
AACTTCTCCTGTTAAAGGCATAATAAAGTGCTTAATACTTTTGTTTCCTCAGCACCCTCTCATTTAATTATATAA
TTTTAGTTCTGAAAGGGACCTATACCAGATGCCTAGAGGAAATTTCAAAACTATGATCTAATGAAAAAATATTTA
ATAGTTCTCCATGCAAATACAAATCATATAGTTTTCCAGAAAATACCTTTGACATTATACAAAGATGATTATCAC
AGCATTATAATAGTAAAAAAATGGAAATAGCCTCTTTCTTCTGTTCTGTTCATAGCACAGTGCCTCATACGCAGT
AGGTTATTATTACATGGTAACTGGCTACCCCAACTGATTAGGAAAGAAGTAAATTTGTTTTATAAAAATACATAC
TCATTGAGGTGCATAGAATAATTAAGAAATTAAAAGACACTTGTAATTTTGAATCCAGTGAATACCCACTGTTAA
TATTTGGTATATCTCTTTCTAGTCTTTTTTTCCCTTTTGCATGTATTTTCTTTAAGACTCCCACCCCCACTGGAT
CATCTCTGCATGTTCTAATCTGCTTTTTTCACAGCAGATTCTAAGCCTCTTTGAATATCAACACAAACTTCAACA
ACTTCATCTATAGATGCCAAATAATAAATTCATTTTTATTTACTTAACCACTTCCTTTGGATGCTTAGGTCATTC
TGATGTTTTGCTATTGAAACCAATGCTATACTGAACACTTCTGTCACTAAAACTTTGCACACACTCATGAATAGC
TTCTTAGGATAAATTTTTAGAGATGGATTTGCTAAATCAGAGACCATTTTTTAAAATTAAAAAACAATTATTCAT
ATCGTTTGGCATGTAAGACAGTAAATTTTCCTTTTATTTTGACAGGATTCAACTGGAAGCTTTGTGCTGCCTTTC
CGGCAAGTCATGTATGCTCCATATCCCACCACACACATAGATGTGGATGTCAATACTGTGAAGCAGATGCCACCC
TGTCATGAACATATTTATAATCAGCGTAGATACATGAGATCCGAGCTGACAGCCTTCTGGAGAGCCACTTCAGAA
GAAGACATGGCTCAGGATACGATCATCTACACTGACGAAAGCTTTACTCCTGATTTGTACGTAATGCTCTGCCTG
CTGGTACTGTAGTCAAGCAATATGAAATTGTGTCTTTTACGAATAAAAACAAAACAGAAGTTGCATTTAAAAAGA
AAGAAATATTACCAGCAGAATTATGCTTGAAGAAACATTTAATCAAGCATTTTTTTCTTAAATGTTCTTCTTTTT
CCATACAATTGTGTTTACCCTAAAATAGGTAAGATTAACCCTTAAAGTAAATATTTAACTATTTGTTTAATAAAT
ATATATTGAGCTCCTAGGCACTGTTCTAGGTACCGGGCTTAATAGTGGCCAACCAGACAGCCCCAGCCCCAGCCC
CTACATTGTGTATAGTCTATTATGTAACAGTTATTGAATGGACTTATTAACAAAACCAAAGAAGTAATTCTAAGT
CTTTTTTTTCTTGACATATGAATATAAAATACAGCAAAACTGTTAAAATATATTAATGGAACATTTTTTTACTTT
GCATTTTATATTGTTATTCACTTCTTATTTTTTTTTAAAAAAAAAAGCCTGAACAGTAAATTCAAAAGGAAAAGT
AATGATAATTAATTGTTGAGCATGGACCCAACTTGAAAAAAAAAATGATGATGATAAATCTATAATCCTAAAACC
CTAAGTAAACACTTAAAAGATGTTCTGAAATCAGGAAAAGAATTATAGTATACTTTTGTGTTTCTCTTTTATCAG
TTGAAAAAAGGCACAGTAGCTCATGCCTGTAAGAACAGAGCTTTGGGAGTGCAAGGCAGGCGGATCACTTGAGGC
CAGGAGTTCCAGACCAGCCTGGGCAACATAGTGAAACCCCATCTCTACAAAAAATAAAAAAGAATTATTGGAATG
TGTTTCTGTGTGCCTGTAATCCTAGCTATTCCGAAAGCTGAGGCAGGAGGATCTTTTGAGCCCAGGAGTTTGAGG
TTACAGGGAGTTATGATGTGCCAGTGTACTCCAGCCTGGGGAACACCGAGACTCTGTCTTATTTAAAAAAAAAAA
AAAAAAAATGCTTGCAATAATGCCTGGCACATAGAAGGTAACAGTAAGTGTTAACTGTAATAACCCAGGTCTAAG
TGTGTAAGGCAATAGAAAAATTGGGGCAAATAAGCCTGACCTATGTATCTACAGAATCAGTTTGAGCTTAGGTAA
CAGACCTGTGGAGCACCAGTAATTACACAGTAAGTGTTAACCAAAAGCATAGAATAGGAATATCTTGTTCAAGGG
ACCCCCAGCCTTATACATCTCAAGGTGCAGAAAGATGACTTAATATAGGACCCATTTTTTCCTAGTTCTCCAGAG
TTTTTATTGGTTCTTGAGAAAGTAGTAGGGGAATGTTTTAGAAAATGAATTGGTCCAACTGAAATTACATGTCAG
TAAGTTTTTATATATTGGTAAATTTTAGTAGACATGTAGAAGTTTTCTAATTAATCTGTGCCTTGAAACATTTTC
TTTTTTCCTAAAGTGCTTAGTATTTTTTCCGTTTTTTGATTGGTTACTTGGGAGCTTTTTTGAGGAAATTTAGTG
AACTGCAGAATGGGTTTGCAACCATTTGGTATTTTTGTTTTGTTTTTTAGAGGATGTATGTGTATTTTAACATTT
CTTAATCATTTTTAGCCAGCTATGTTTGTTTTGCTGATTTGACAAACTACAGTTAGACAGCTATTCTCATTTTGC
TGATCATGACAAAATAATATCCTGAATTTTTAAATTTTGCATCCAGCTCTAAATTTTCTAAACATAAAATTGTCC
AAAAAATAGTATTTTCAGCCACTAGATTGTGTGTTAAGTCTATTGTCACAGAGTCATTTTACTTTTAAGTATATG
TTTTTACATGTTAATTATGTTTGTTATTTTTAATTTTAACTTTTTAAAATAATTCCAGTCACTGCCAATACATGA
AAAATTGGTCACTGGAATTTTTTTTTTGACTTTTATTTTAGGTTCATGTGTACATGTGCAGGTGTGTTATACAGG
TAAATTGCGTGTCATGAGGGTTTGGTGTACAGGTGATTTCATTACCCAGGTAATAAGCATAGTACCCAATAGGTA
GTTTTTTGATCCTCACCCTTCTCCCACCCTCAAGTAGGCCCTGGTGTTGCTGTTTCCTTCTTTGTGTCCATGTAT
ACTCAGTGTTTAGCTCCCACTTAGAAGTGAGAACATGCGGTAGTTGGTTTTCTGTTCCTGGATTAGTTCACTTAG
GATAATGACCTCTAGCTCCATCTGGTTTTTATGGCTGCATAGTATTCCATGGTGTATATGTATCACATTTTCTTT
ATCCAGTCTACCATTGATAGGCATTTAGGTTGATTCCCTGTCTTTGTTATCATGAATAGTGCTGTGATGAACATA
CACATGCATGTGTCTTTATGGTAGAAAAATTTGTATTCCTTTAGGTACATATAGAATAATGGGGTTGCTAGGGTG
AATGGTAGTTCTATTTTCAGTTATTTGAGAAATCTTCAAACTGCTTTTCATAATAGCTAAACTAATTTACAGTCC
CGCCAGCAGTGTATAAGTGTTCCCTTTTCTCCACAACCTTGCCAACATCTGTGATTTTTTGACTTTTTAATAATA
GCCATTCCTAGAGAATTGATTTGCAATTCTCTATTAGTGATATTAAGCATTTTTTCATATGCTTTTTAGCTGTCT
GTATATATTCTTCTGAAAAATTTTCATGTCCTTTGCCCAGTTTGTAGTGGGGTGGGTTGTTTTTTGCTTGTTAAT
TAGTTTTAAGTTCCTTCCAGATTCTGCATATCCCTTTGTTGGATACATGGTTTGCAGATATTTTTCTCCCATTGT
GTAGGTTGTCTTTTACTCTGTTGATAGTTTCTTTTGCCATGCAGGAGCTCGTTAGGTCCCATTTGTGTTTGTTTT
TGTTGCAGTTGCTTTTGGCGTCTTCATCATAAAATCTGTGCCAGGGCCTATGTCCAGAATGGTATTTCCTAGGTT
GTCTTCCAGGGTTTTTACAATTTTAGATTTTACGTTTATGTCTTTAATCCATCTTGAGTTGATTTTTGTATATGG
CACAAGGAAGGGGTCCAGTTTCACTCCAATTCCTATGGCTAGCAATTATCCCAGCACCATTTATTGAATACGGAG
TCCTTTCCCCATTGCTTGTTTTTTGTCAACTTTGTTGAAGATCAGATGGTTGTAAGTGTGTGGCTTTATTTCTTG
GCTCTCTATTCTCCATTGGTCTATGTGTCTGTTTTTATAACAGTACCCTGCTGTTCAGGTTCCTATAGCCTTTTA
GTATAAAATCGGCTAATGTGATGCCTCCAGCTTTGTTCTTTTTGCTTAGGATTGCTTTGGCTATTTGGGCTCCTT
TTTGGGTCCATATTAATTTTAAAACAGTTTTTTCTGGTTTTGTGAAGGATATCATTGGTAGTTTATAGGAATAGC
ATTGAATCTGTAGATTGCTTTGGGCAGTATGGCCATTTTAACAATATTAATTCTTCCTATCTATGAATATGGAAT
GTTTTTCCATGTGTTTGTGTCATCTCTTTATACCTGATGTATAAAGAAAAGCTGGTATTATTCCTACTCAATCTG
TTCCAAAAAATTGAGGAGGAGGAACTCTTCCCTAATGAGGCCAGCATCATTCTGATACCAAAACCTGGCAGAGAC
ACAACAGAAAAAAGAAAACTTCAGGCCAATATCCTTGATGAATATAGATGCAAAAATCCTCAACAAAATACTAGC
AAACCAAATCCAGCAGCACATCAAAAAGCTGATCTACTTTGATCAAGTAGGCTTTATCCCTGGGATGCAAGGTTG
GTTCAACATACACAAATCAATAAGTGTGATTCATCACATAAACAGAGCTAAAAACAAAAACCACAAGATTATCTC
AATAGGTAGAGAAAAGGTTGTCAATAAAATTTAACATCCTCCATGTTAAAAACCTTCAGTAGGTCAGGTGTAGTG
ACTCACACCTGTAATCCCAGCACTTTGGGAGGCCAAGGCGGGCATATCTCTTAAGCCCAGGAGTTCAAGACGAGC
CTAGGCAGCATGGTGAAACCCCATCTCTACAAAAAAAAAAAAAAAAAAAAATTAGCTTGGTATGGTGACATGCAC
CTATAGTCCCAGCTATTCAGGAGGTTGAGGTGGGAGGATTGTTTGAGCCCGGGAGGCAGAGGTTGGCAGCGAGCT
GAGATCATGCCACCGCACTCCAGCCTGGGCAACGGAGTGAGACCCTGTCTCAAAAAAGAAAAATCACAAACAATC
CTAAACAAACTAGGCATTGAAGGAACATGCCTCAAAAAAATAAGAACCATCTATGACAGACCCATAGCCAATATC
TTACCAAATGGGCAAAAGCTGGAAGTATTCTCCTTGAGAACCGTAACAAGACAAGGATGTCCACTCTCACCACTC
CTTTTCAGCATAGTTCTGGAAGTCCTAGCCAGAGCAATCAGGAAAGAGAAAGAAAGAAAGACATTCAGATAGGAA
GAGAAGAAGTCAAACTATTTCTGTTTGCAGGCAGTATAATTCTGTACCTAGAAAATCTCATAGTCTCTGCCCAGA
AACTCCTAAATCTGTTAAAAATTTCAGCAAAGTTTTGGCATTCTCTATACTCCAACACCTTCCAAAGTGAGAGCA
AAATCAAGAACACAGTCCCATTCACAATAGCCGCAAAACGAATAAAATACCTAGGAATCCAGCTAACCAGGGAGG
TGAAAGATCTCTATGAGAATTACAAAACACTGCTGAAAGAAATCAGAGATGACACAAACAAATGGAAATGTTCTT
TTTTAACACCTTGCTTTATCTAATTCACTTATGATGAAGATACTCATTCAGTGGAACAGGTATAATAAGTCCACT
CGATTAAATATAAGCCTTATTCTCTTTCCAGAGCCCAAGAAGGGGCACTATCAGTGCCCAGTCAATAATGACGAA
ATGCTAATATTTTTCCCCTTTACGGTTTCTTTCTTCTGTAGTGTGGTACACTCGTTTCTTAAGATAAGGAAACTT
GAACTACCTTCCTGTTTGCTTCTACACATACCCATTCTCTTTTTTTGCCACTCTGGTCAGGTATAGGATGATCCC
TACCACTTTCAGTTAAAAACTCCTCCTCTTACTAAATGTTCTCTTACCCTCTGGCCTGAGTAGAACCTAGGGAAA
ATGGAAGAGAAAAAGATGAAAGGGAGGTGGGGCCTGGGAAGGGAATAAGTAGTCCTGTTTGTTTGTGTGTTTGCT
TTAGCACCTGCTATATCCTAGGTGCTGTGTTAGGCACACATTATTTTAAGTGGCCATTATATTACTACTACTCAC
TCTGGTCGTTGCCAAGGTAGGTAGTACTTTCTTGGATAGTTGGTTCATGTTACTTACAGATGGTGGGCTTGTTGA
GGCAAACCCAGTGGATAATCATCGGAGTGTGTTCTCTAATCTCACTCAAATTTTTCTTCACATTTTTTGGTTTGT
TTTGGTTTTTGATGGTAGTGGCTTATTTTTGTTGCTGGTTTGTTTTTTGTTTTTTTTTGAGATGGCAAGAATTGG
TAGTTTTATTTATTAATTGCCTAAGGGTCTCTACTTTTTTTAAAAGATGAGAGTAGTAAAATAGATTGATAGATA
CATACATACCCTTACTGGGGACTGCTTATATTCTTTAGAGAAAAAATTACATATTAGCCTGACAAACACCAGTAA
AATGTAAATATATCCTTGAGTAAATAAATGAATGTATATTTTGTGTCTCCAAATATATATATCTATATTCTTACA
AATGTGTTTATATGTAATATCAATTTATAAGAACTTAAAATGTTGGCTCAAGTGAGGGATTGTGGAAGGTAGCAT
TATATGGCCATTTCAACATTTGAACTTTTTTCTTTTCTTCATTTTCTTCTTTTCTTCAGGAATATTTTTCAAGAT
GTCTTACACAGAGACACTCTAGTGAAAGCCTTCCTGGATCAGGTAAATGTTGAACTTGAGATTGTCAGAGTGAAT
GATATGACATGTTTTCTTTTTTAATATATCCTACAATGCCTGTTCTATATATTTATATTCCCCTGGATCATGCCC
CAGAGTTCTGCTCAGCAATTGCAGTTAAGTTAGTTACACTACAGTTCTCAGAAGAGTCTGTGAGGGCATGTCAAG
TGCATCATTACATTGGTTGCCTCTTGTCCTAGATTTATGCTTCGGGAATTCAGACCTTTGTTTACAATATAATAA
ATATTATTGCTATCTTTTAAAGATATAATAATAAGATATAAAGTTGACCACAACTACTGTTTTTTGAAACATAGA
ATTCCTGGTTTACATGTATCAAAGTGAAATCTGACTTAGCTTTTACAGATATAATATATACATATATATATCCTG
CAATGCTTGTACTATATATGTAGTACAAGTATATATATATGTTTGTGTGTGTATATATATATAGTACGAGCATAT
ATACATATTACCAGCATTGTAGGATATATATATGTTTATATATTAAAAAAAAGTTATAAACTTAAAACCCTATTA
TGTTATGTAGAGTATATGTTATATATGATATGTAAAATATATAACATATACTCTATGATAGAGTGTAATATATTT
TTTATATATATTTTAACATTTATAAAATGATAGAATTAAGAATTGAGTCCTAATCTGTTTTATTAGGTGCTTTTT
GTAGTGTCTGGTCTTTCTAAAGTGTCTAAATGATTTTTCCTTTTGACTTATTAATGGGGAAGAGCCTGTATATTA
ACAATTAAGAGTGCAGCATTCCATACGTCAAACAACAAACATTTTAATTCAAGCATTAACCTATAACAAGTAAGT
TTTTTTTTTTTTTTTGAGAAAGGGAGGTTGTTTATTTGCCTGAAATGACTCAAAAATATTTTTGAAACATAGTGT
ACTTATTTAAATAACATCTTTATTGTTTCATTCTTTTAAAAAATATCTACTTAATTACACAGTTGAAGGAAATCG
TAGATTATATGGAACTTATTTCTTAATATATTACAGTTTGTTATAATAACATTCTGGGGATCAGGCCAGGAAACT
GTGTCATAGATAAAGCTTTGAAATAATGAGATCCTTATGTTTACTAGAAATTTTGGATTGAGATCTATGAGGTCT
GTGACATATTGCGAAGTTCAAGGAAAATTCGTAGGCCTGGAATTTCATGCTTCTCAAGCTGACATAAAATCCCTC
CCACTCTCCACCTCATCATATGCACACATTCTACTCCTACCCACCCACTCCACCCCCTGCAAAAGTACAGGTATA
TGAATGTCTCAAAACCATAGGCTCATCTTCTAGGAGCTTCAATGTTATTTGAAGATTTGGGCAGAAAAAATTAAG
TAATACGAAATAACTTATGTATGAGTTTTAAAAGTGAAGTAAACATGGATGTATTCTGAAGTAGAATGCAAAATT
TGAATGCATTTTTAAAGATAAATTAGAAAACTTCTAAAAACTGTCAGATTGTCTGGGCCTGGTGGCTTATGCCTG
TAATCCCAGCACTTTGGGAGTCCGAGGTGGGTGGATCACAAGGTCAGGAGATCGAGACCATCCTGCCAACATGGT
GAAACCCCGTCTCTACTAAGTATACAAAAATTAGCTGGGCGTGGCAGCGTGTGCCTGTAATCCCAGCTACCTGGG
AGGCTGAGGCAGGAGAATCGCTTGAACCCAGGAGGTGTAGGTTGCAGTGAGTCAAGATCGCGCCACTGCACTTTA
GCCTGGTGACAGAGCTAGACTCCGTCTCAAAAAAAAAAAAAAATATCAGATTGTTCCTACACCTAGTGCTTCTAT
ACCACACTCCTGTTAGGGGGCATCAGTGGAAATGGTTAAGGAGATGTTTAGTGTGTATTGTCTGCCAAGCACTGT
CAACACTGTCATAGAAACTTCTGTACGAGTAGAATGTGAGCAAATTATGTGTTGAAATGGTTCCTCTCCCTGCAG
GTCTTTCAGCTGAAACCTGGCTTATCTCTCAGAAGTACTTTCCTTGCACAGTTTCTACTTGTCCTTCACAGAAAA
GCCTTGACACTAATAAAATATATAGAAGACGATACGTGAGTAAAACTCCTACACGGAAGAAAAACCTTTGTACAT
TGTTTTTTTGTTTTGTTTCCTTTGTACATTTTCTATATCATAATTTTTGCGCTTCTTTTTTTTTTTTTTTTTTTT
TTTTTTCCATTATTTTTAGGCAGAAGGGAAAAAAGCCCTTTAAATCTCTTCGGAACCTGAAGATAGACCTTGATT
TAACAGCAGAGGGCGATCTTAACATAATAATGGCTCTGGCTGAGAAAATTAAACCAGGCCTACACTCTTTTATCT
TTGGAAGACCTTTCTACACTAGTGTGCAAGAACGAGATGTTCTAATGACTTTTTAAATGTGTAACTTAATAAGCC
TATTCCATCACAATCATGATCGCTGGTAAAGTAGCTCAGTGGTGTGGGGAAACGTTCCCCTGGATCATACTCCAG
AATTCTGCTCTCAGCAATTGCAGTTAAGTAAGTTACACTACAGTTCTCACAAGAGCCTGTGAGGGGATGTCAGGT
GCATCATTACATTGGGTGTCTCTTTTCCTAGATTTATGCTTTTGGGATACAGACCTATGTTTACAATATAATAAA
TATTATTGCTATCTTTTAAAGATATAATAATAGGATGTAAACTTGACCACAACTACTGTTTTTTTGAAATACATG
ATTCATGGTTTACATGTGTCAAGGTGAAATCTGAGTTGGCTTTTACAGATAGTTGACTTTCTATCTTTTGGCATT
CTTTGGTGTGTAGAATTACTGTAATACTTCTGCAATCAACTGAAAACTAGAGCCTTTAAATGATTTCAATTCCAC
AGAAAGAAAGTGAGCTTGAACATAGGATGAGCTTTAGAAAGAAAATTGATCAAGCAGATGTTTAATTGGAATTGA
TTATTAGATCCTACTTTGTGGATTTAGTCCCTGGGATTCAGTCTGTAGAAATGTCTAATAGTTCTCTATAGTCCT
TGTTCCTGGTGAACCACAGTTAGGGTGTTTTGTTTATTTTATTGTTCTTGCTATTGTTGATATTCTATGTAGTTG
AGCTCTGTAAAAGGAAATTGTATTTTATGTTTTAGTAATTGTTGCCAACTTTTTAAATTAATTTTCATTATTTTT
GAGCCAAATTGAAATGTGCACCTCCTGTGCCTTTTTTCTCCTTAGAAAATCTAATTACTTGGAACAAGTTCAGAT
TTCACTGGTCAGTCATTTTCATCTTGTTTTCTTCTTGCTAAGTCTTACCATGTACCTGCTTTGGCAATCATTGCA
ACTCTGAGATTATAAAATGCCTTAGAGAATATACTAACTAATAAGATCTTTTTTTCAGAAACAGAAAATAGTTCC
TTGAGTACTTCCTTCTTGCATTTCTGCCTATGTTTTTGAAGTTGTTGCTGTTTGCCTGCAATAGGCTATAAGGAA
TAGCAGGAGAAATTTTACTGAAGTGCTGTTTTCCTAGGTGCTACTTTGGCAGAGCTAAGTTATCTTTTGTTTTCT
TAATGCGTTTGGACCATTTTGCTGGCTATAAAATAACTGATTAATATAATTCTAACACAATGTTGACATTGTAGT
TACACAAACACAAATAAATATTTTATTTAAAATTCTGGAAGTAATATAAAAGGGAAAATATATTTATAAGAAAGG
GATAAAGGTAATAGAGCCCTTCTGCCCCCCACCCACCAAATTTACACAACAAAATGACATGTTCGAATGTGAAAG
GTCATAATAGCTTTCCCATCATGAATCAGAAAGATGTGGACAGCTTGATGTTTTAGACAACCACTGAACTAGATG
ACTGTTGTACTGTAGCTCAGTCATTTAAAAAATATATAAATACTACCTTGTAGTGTCCCATACTGTGTTTTTTAC
ATGGTAGATTCTTATTTAAGTGCTAACTGGTTATTTTCTTTGGCTGGTTTATTGTACTGTTATACAGAATGTAAG
TTGTACAGTGAAATAAGTTATTAAAGCATGTGTAAACATTGTTATATATCTTTTCTCCTAAATGGAGAATTTTGA
ATAAAATATATTTGAAATTTT
SEQ ID NO: 14
Reverse Complement of SEQ ID NO: 13
AAAATTTCAAATATATTTTATTCAAAATTCTCCATTTAGGAGAAAAGATATATAACAATGTTTACACATGCTTTA
ATAACTTATTTCACTGTACAACTTACATTCTGTATAACAGTACAATAAACCAGCCAAAGAAAATAACCAGTTAGC
ACTTAAATAAGAATCTACCATGTAAAAAACACAGTATGGGACACTACAAGGTAGTATTTATATATTTTTTAAATG
ACTGAGCTACAGTACAACAGTCATCTAGTTCAGTGGTTGTCTAAAACATCAAGCTGTCCACATCTTTCTGATTCA
TGATGGGAAAGCTATTATGACCTTTCACATTCGAACATGTCATTTTGTTGTGTAAATTTGGTGGGTGGGGGGCAG
AAGGGCTCTATTACCTTTATCCCTTTCTTATAAATATATTTTCCCTTTTATATTACTTCCAGAATTTTAAATAAA
ATATTTATTTGTGTTTGTGTAACTACAATGTCAACATTGTGTTAGAATTATATTAATCAGTTATTTTATAGCCAG
CAAAATGGTCCAAACGCATTAAGAAAACAAAAGATAACTTAGCTCTGCCAAAGTAGCACCTAGGAAAACAGCACT
TCAGTAAAATTTCTCCTGCTATTCCTTATAGCCTATTGCAGGCAAACAGCAACAACTTCAAAAACATAGGCAGAA
ATGCAAGAAGGAAGTACTCAAGGAACTATTTTCTGTTTCTGAAAAAAAGATCTTATTAGTTAGTATATTCTCTAA
GGCATTTTATAATCTCAGAGTTGCAATGATTGCCAAAGCAGGTACATGGTAAGACTTAGCAAGAAGAAAACAAGA
TGAAAATGACTGACCAGTGAAATCTGAACTTGTTCCAAGTAATTAGATTTTCTAAGGAGAAAAAAGGCACAGGAG
GTGCACATTTCAATTTGGCTCAAAAATAATGAAAATTAATTTAAAAAGTTGGCAACAATTACTAAAACATAAAAT
ACAATTTCCTTTTACAGAGCTCAACTACATAGAATATCAACAATAGCAAGAACAATAAAATAAACAAAACACCCT
AACTGTGGTTCACCAGGAACAAGGACTATAGAGAACTATTAGACATTTCTACAGACTGAATCCCAGGGACTAAAT
CCACAAAGTAGGATCTAATAATCAATTCCAATTAAACATCTGCTTGATCAATTTTCTTTCTAAAGCTCATCCTAT
GTTCAAGCTCACTTTCTTTCTGTGGAATTGAAATCATTTAAAGGCTCTAGTTTTCAGTTGATTGCAGAAGTATTA
CAGTAATTCTACACACCAAAGAATGCCAAAAGATAGAAAGTCAACTATCTGTAAAAGCCAACTCAGATTTCACCT
TGACACATGTAAACCATGAATCATGTATTTCAAAAAAACAGTAGTTGTGGTCAAGTTTACATCCTATTATTATAT
CTTTAAAAGATAGCAATAATATTTATTATATTGTAAACATAGGTCTGTATCCCAAAAGCATAAATCTAGGAAAAG
AGACACCCAATGTAATGATGCACCTGACATCCCCTCACAGGCTCTTGTGAGAACTGTAGTGTAACTTACTTAACT
GCAATTGCTGAGAGCAGAATTCTGGAGTATGATCCAGGGGAACGTTTCCCCACACCACTGAGCTACTTTACCAGC
GATCATGATTGTGATGGAATAGGCTTATTAAGTTACACATTTAAAAAGTCATTAGAACATCTCGTTCTTGCACAC
TAGTGTAGAAAGGTCTTCCAAAGATAAAAGAGTGTAGGCCTGGTTTAATTTTCTCAGCCAGAGCCATTATTATGT
TAAGATCGCCCTCTGCTGTTAAATCAAGGTCTATCTTCAGGTTCCGAAGAGATTTAAAGGGCTTTTTTCCCTTCT
GCCTAAAAATAATGGAAAAAAAAAAAAAAAAAAAAAAAAAAGAAGCGCAAAAATTATGATATAGAAAATGTACAA
AGGAAACAAAACAAAAAAACAATGTACAAAGGTTTTTCTTCCGTGTAGGAGTTTTACTCACGTATCGTCTTCTAT
ATATTTTATTAGTGTCAAGGCTTTTCTGTGAAGGACAAGTAGAAACTGTGCAAGGAAAGTACTTCTGAGAGATAA
GCCAGGTTTCAGCTGAAAGACCTGCAGGGAGAGGAACCATTTCAACACATAATTTGCTCACATTCTACTCGTACA
GAAGTTTCTATGACAGTGTTGACAGTGCTTGGCAGACAATACACACTAAACATCTCCTTAACCATTTCCACTGAT
GCCCCCTAACAGGAGTGTGGTATAGAAGCACTAGGTGTAGGAACAATCTGATATTTTTTTTTTTTTTTGAGACGG
AGTCTAGCTCTGTCACCAGGCTAAAGTGCAGTGGCGCGATCTTGACTCACTGCAACCTACACCTCCTGGGTTCAA
GCGATTCTCCTGCCTCAGCCTCCCAGGTAGCTGGGATTACAGGCACACGCTGCCACGCCCAGCTAATTTTTGTAT
ACTTAGTAGAGACGGGGTTTCACCATGTTGGCAGGATGGTCTCGATCTCCTGACCTTGTGATCCACCCACCTCGG
ACTCCCAAAGTGCTGGGATTACAGGCATAAGCCACCAGGCCCAGACAATCTGACAGTTTTTAGAAGTTTTCTAAT
TTATCTTTAAAAATGCATTCAAATTTTGCATTCTACTTCAGAATACATCCATGTTTACTTCACTTTTAAAACTCA
TACATAAGTTATTTCGTATTACTTAATTTTTTCTGCCCAAATCTTCAAATAACATTGAAGCTCCTAGAAGATGAG
CCTATGGTTTTGAGACATTCATATACCTGTACTTTTGCAGGGGGTGGAGTGGGTGGGTAGGAGTAGAATGTGTGC
ATATGATGAGGTGGAGAGTGGGAGGGATTTTATGTCAGCTTGAGAAGCATGAAATTCCAGGCCTACGAATTTTCC
TTGAACTTCGCAATATGTCACAGACCTCATAGATCTCAATCCAAAATTTCTAGTAAACATAAGGATCTCATTATT
TCAAAGCTTTATCTATGACACAGTTTCCTGGCCTGATCCCCAGAATGTTATTATAACAAACTGTAATATATTAAG
AAATAAGTTCCATATAATCTACGATTTCCTTCAACTGTGTAATTAAGTAGATATTTTTTAAAAGAATGAAACAAT
AAAGATGTTATTTAAATAAGTACACTATGTTTCAAAAATATTTTTGAGTCATTTCAGGCAAATAAACAACCTCCC
TTTCTCAAAAAAAAAAAAAAAACTTACTTGTTATAGGTTAATGCTTGAATTAAAATGTTTGTTGTTTGACGTATG
GAATGCTGCACTCTTAATTGTTAATATACAGGCTCTTCCCCATTAATAAGTCAAAAGGAAAAATCATTTAGACAC
TTTAGAAAGACCAGACACTACAAAAAGCACCTAATAAAACAGATTAGGACTCAATTCTTAATTCTATCATTTTAT
AAATGTTAAAATATATATAAAAAATATATTACACTCTATCATAGAGTATATGTTATATATTTTACATATCATATA
TAACATATACTCTACATAACATAATAGGGTTTTAAGTTTATAACTTTTTTTTAATATATAAACATATATATATCC
TACAATGCTGGTAATATGTATATATGCTCGTACTATATATATATACACACACAAACATATATATATACTTGTACT
ACATATATAGTACAAGCATTGCAGGATATATATATGTATATATTATATCTGTAAAAGCTAAGTCAGATTTCACTT
TGATACATGTAAACCAGGAATTCTATGTTTCAAAAAACAGTAGTTGTGGTCAACTTTATATCTTATTATTATATC
TTTAAAAGATAGCAATAATATTTATTATATTGTAAACAAAGGTCTGAATTCCCGAAGCATAAATCTAGGACAAGA
GGCAACCAATGTAATGATGCACTTGACATGCCCTCACAGACTCTTCTGAGAACTGTAGTGTAACTAACTTAACTG
CAATTGCTGAGCAGAACTCTGGGGCATGATCCAGGGGAATATAAATATATAGAACAGGCATTGTAGGATATATTA
AAAAAGAAAACATGTCATATCATTCACTCTGACAATCTCAAGTTCAACATTTACCTGATCCAGGAAGGCTTTCAC
TAGAGTGTCTCTGTGTAAGACATCTTGAAAAATATTCCTGAAGAAAAGAAGAAAATGAAGAAAAGAAAAAAGTTC
AAATGTTGAAATGGCCATATAATGCTACCTTCCACAATCCCTCACTTGAGCCAACATTTTAAGTTCTTATAAATT
GATATTACATATAAACACATTTGTAAGAATATAGATATATATATTTGGAGACACAAAATATACATTCATTTATTT
ACTCAAGGATATATTTACATTTTACTGGTGTTTGTCAGGCTAATATGTAATTTTTTCTCTAAAGAATATAAGCAG
TCCCCAGTAAGGGTATGTATGTATCTATCAATCTATTTTACTACTCTCATCTTTTAAAAAAAGTAGAGACCCTTA
GGCAATTAATAAATAAAACTACCAATTCTTGCCATCTCAAAAAAAAACAAAAAACAAACCAGCAACAAAAATAAG
CCACTACCATCAAAAACCAAAACAAACCAAAAAATGTGAAGAAAAATTTGAGTGAGATTAGAGAACACACTCCGA
TGATTATCCACTGGGTTTGCCTCAACAAGCCCACCATCTGTAAGTAACATGAACCAACTATCCAAGAAAGTACTA
CCTACCTTGGCAACGACCAGAGTGAGTAGTAGTAATATAATGGCCACTTAAAATAATGTGTGCCTAACACAGCAC
CTAGGATATAGCAGGTGCTAAAGCAAACACACAAACAAACAGGACTACTTATTCCCTTCCCAGGCCCCACCTCCC
TTTCATCTTTTTCTCTTCCATTTTCCCTAGGTTCTACTCAGGCCAGAGGGTAAGAGAACATTTAGTAAGAGGAGG
AGTTTTTAACTGAAAGTGGTAGGGATCATCCTATACCTGACCAGAGTGGCAAAAAAAGAGAATGGGTATGTGTAG
AAGCAAACAGGAAGGTAGTTCAAGTTTCCTTATCTTAAGAAACGAGTGTACCACACTACAGAAGAAAGAAACCGT
AAAGGGGAAAAATATTAGCATTTCGTCATTATTGACTGGGCACTGATAGTGCCCCTTCTTGGGCTCTGGAAAGAG
AATAAGGCTTATATTTAATCGAGTGGACTTATTATACCTGTTCCACTGAATGAGTATCTTCATCATAAGTGAATT
AGATAAAGCAAGGTGTTAAAAAAGAACATTTCCATTTGTTTGTGTCATCTCTGATTTCTTTCAGCAGTGTTTTGT
AATTCTCATAGAGATCTTTCACCTCCCTGGTTAGCTGGATTCCTAGGTATTTTATTCGTTTTGCGGCTATTGTGA
ATGGGACTGTGTTCTTGATTTTGCTCTCACTTTGGAAGGTGTTGGAGTATAGAGAATGCCAAAACTTTGCTGAAA
TTTTTAACAGATTTAGGAGTTTCTGGGCAGAGACTATGAGATTTTCTAGGTACAGAATTATACTGCCTGCAAACA
GAAATAGTTTGACTTCTTCTCTTCCTATCTGAATGTCTTTCTTTCTTTCTCTTTCCTGATTGCTCTGGCTAGGAC
TTCCAGAACTATGCTGAAAAGGAGTGGTGAGAGTGGACATCCTTGTCTTGTTACGGTTCTCAAGGAGAATACTTC
CAGCTTTTGCCCATTTGGTAAGATATTGGCTATGGGTCTGTCATAGATGGTTCTTATTTTTTTGAGGCATGTTCC
TTCAATGCCTAGTTTGTTTAGGATTGTTTGTGATTTTTCTTTTTTGAGACAGGGTCTCACTCCGTTGCCCAGGCT
GGAGTGCGGTGGCATGATCTCAGCTCGCTGCCAACCTCTGCCTCCCGGGCTCAAACAATCCTCCCACCTCAACCT
CCTGAATAGCTGGGACTATAGGTGCATGTCACCATACCAAGCTAATTTTTTTTTTTTTTTTTTTTTGTAGAGATG
GGGTTTCACCATGCTGCCTAGGCTCGTCTTGAACTCCTGGGCTTAAGAGATATGCCCGCCTTGGCCTCCCAAAGT
GCTGGGATTACAGGTGTGAGTCACTACACCTGACCTACTGAAGGTTTTTAACATGGAGGATGTTAAATTTTATTG
ACAACCTTTTCTCTACCTATTGAGATAATCTTGTGGTTTTTGTTTTTAGCTCTGTTTATGTGATGAATCACACTT
ATTGATTTGTGTATGTTGAACCAACCTTGCATCCCAGGGATAAAGCCTACTTGATCAAAGTAGATCAGCTTTTTG
ATGTGCTGCTGGATTTGGTTTGCTAGTATTTTGTTGAGGATTTTTGCATCTATATTCATCAAGGATATTGGCCTG
AAGTTTTCTTTTTTCTGTTGTGTCTCTGCCAGGTTTTGGTATCAGAATGATGCTGGCCTCATTAGGGAAGAGTTC
CTCCTCCTCAATTTTTTGGAACAGATTGAGTAGGAATAATACCAGCTTTTCTTTATACATCAGGTATAAAGAGAT
GACACAAACACATGGAAAAACATTCCATATTCATAGATAGGAAGAATTAATATTGTTAAAATGGCCATACTGCCC
AAAGCAATCTACAGATTCAATGCTATTCCTATAAACTACCAATGATATCCTTCACAAAACCAGAAAAAACTGTTT
TAAAATTAATATGGACCCAAAAAGGAGCCCAAATAGCCAAAGCAATCCTAAGCAAAAAGAACAAAGCTGGAGGCA
TCACATTAGCCGATTTTATACTAAAAGGCTATAGGAACCTGAACAGCAGGGTACTGTTATAAAAACAGACACATA
GACCAATGGAGAATAGAGAGCCAAGAAATAAAGCCACACACTTACAACCATCTGATCTTCAACAAAGTTGACAAA
AAACAAGCAATGGGGAAAGGACTCCGTATTCAATAAATGGTGCTGGGATAATTGCTAGCCATAGGAATTGGAGTG
AAACTGGACCCCTTCCTTGTGCCATATACAAAAATCAACTCAAGATGGATTAAAGACATAAACGTAAAATCTAAA
ATTGTAAAAACCCTGGAAGACAACCTAGGAAATACCATTCTGGACATAGGCCCTGGCACAGATTTTATGATGAAG
ACGCCAAAAGCAACTGCAACAAAAACAAACACAAATGGGACCTAACGAGCTCCTGCATGGCAAAAGAAACTATCA
ACAGAGTAAAAGACAACCTACACAATGGGAGAAAAATATCTGCAAACCATGTATCCAACAAAGGGATATGCAGAA
TCTGGAAGGAACTTAAAACTAATTAACAAGCAAAAAACAACCCACCCCACTACAAACTGGGCAAAGGACATGAAA
ATTTTTCAGAAGAATATATACAGACAGCTAAAAAGCATATGAAAAAATGCTTAATATCACTAATAGAGAATTGCA
AATCAATTCTCTAGGAATGGCTATTATTAAAAAGTCAAAAAATCACAGATGTTGGCAAGGTTGTGGAGAAAAGGG
AACACTTATACACTGCTGGCGGGACTGTAAATTAGTTTAGCTATTATGAAAAGCAGTTTGAAGATTTCTCAAATA
ACTGAAAATAGAACTACCATTCACCCTAGCAACCCCATTATTCTATATGTACCTAAAGGAATACAAATTTTTCTA
CCATAAAGACACATGCATGTGTATGTTCATCACAGCACTATTCATGATAACAAAGACAGGGAATCAACCTAAATG
CCTATCAATGGTAGACTGGATAAAGAAAATGTGATACATATACACCATGGAATACTATGCAGCCATAAAAACCAG
ATGGAGCTAGAGGTCATTATCCTAAGTGAACTAATCCAGGAACAGAAAACCAACTACCGCATGTTCTCACTTCTA
AGTGGGAGCTAAACACTGAGTATACATGGACACAAAGAAGGAAACAGCAACACCAGGGCCTACTTGAGGGTGGGA
GAAGGGTGAGGATCAAAAAACTACCTATTGGGTACTATGCTTATTACCTGGGTAATGAAATCACCTGTACACCAA
ACCCTCATGACACGCAATTTACCTGTATAACACACCTGCACATGTACACATGAACCTAAAATAAAAGTCAAAAAA
AAAATTCCAGTGACCAATTTTTCATGTATTGGCAGTGACTGGAATTATTTTAAAAAGTTAAAATTAAAAATAACA
AACATAATTAACATGTAAAAACATATACTTAAAAGTAAAATGACTCTGTGACAATAGACTTAACACACAATCTAG
TGGCTGAAAATACTATTTTTTGGACAATTTTATGTTTAGAAAATTTAGAGCTGGATGCAAAATTTAAAAATTCAG
GATATTATTTTGTCATGATCAGCAAAATGAGAATAGCTGTCTAACTGTAGTTTGTCAAATCAGCAAAACAAACAT
AGCTGGCTAAAAATGATTAAGAAATGTTAAAATACACATACATCCTCTAAAAAACAAAACAAAAATACCAAATGG
TTGCAAACCCATTCTGCAGTTCACTAAATTTCCTCAAAAAAGCTCCCAAGTAACCAATCAAAAAACGGAAAAAAT
ACTAAGCACTTTAGGAAAAAAGAAAATGTTTCAAGGCACAGATTAATTAGAAAACTTCTACATGTCTACTAAAAT
TTACCAATATATAAAAACTTACTGACATGTAATTTCAGTTGGACCAATTCATTTTCTAAAACATTCCCCTACTAC
TTTCTCAAGAACCAATAAAAACTCTGGAGAACTAGGAAAAAATGGGTCCTATATTAAGTCATCTTTCTGCACCTT
GAGATGTATAAGGCTGGGGGTCCCTTGAACAAGATATTCCTATTCTATGCTTTTGGTTAACACTTACTGTGTAAT
TACTGGTGCTCCACAGGTCTGTTACCTAAGCTCAAACTGATTCTGTAGATACATAGGTCAGGCTTATTTGCCCCA
ATTTTTCTATTGCCTTACACACTTAGACCTGGGTTATTACAGTTAACACTTACTGTTACCTTCTATGTGCCAGGC
ATTATTGCAAGCATTTTTTTTTTTTTTTTTTTAAATAAGACAGAGTCTCGGTGTTCCCCAGGCTGGAGTACACTG
GCACATCATAACTCCCTGTAACCTCAAACTCCTGGGCTCAAAAGATCCTCCTGCCTCAGCTTTCGGAATAGCTAG
GATTACAGGCACACAGAAACACATTCCAATAATTCTTTTTTATTTTTTGTAGAGATGGGGTTTCACTATGTTGCC
CAGGCTGGTCTGGAACTCCTGGCCTCAAGTGATCCGCCTGCCTTGCACTCCCAAAGCTCTGTTCTTACAGGCATG
AGCTACTGTGCCTTTTTTCAACTGATAAAAGAGAAACACAAAAGTATACTATAATTCTTTTCCTGATTTCAGAAC
ATCTTTTAAGTGTTTACTTAGGGTTTTAGGATTATAGATTTATCATCATCATTTTTTTTTTCAAGTTGGGTCCAT
GCTCAACAATTAATTATCATTACTTTTCCTTTTGAATTTACTGTTCAGGCTTTTTTTTTTAAAAAAAAATAAGAA
GTGAATAACAATATAAAATGCAAAGTAAAAAAATGTTCCATTAATATATTTTAACAGTTTTGCTGTATTTTATAT
TCATATGTCAAGAAAAAAAAGACTTAGAATTACTTCTTTGGTTTTGTTAATAAGTCCATTCAATAACTGTTACAT
AATAGACTATACACAATGTAGGGGCTGGGGCTGGGGCTGTCTGGTTGGCCACTATTAAGCCCGGTACCTAGAACA
GTGCCTAGGAGCTCAATATATATTTATTAAACAAATAGTTAAATATTTACTTTAAGGGTTAATCTTACCTATTTT
AGGGTAAACACAATTGTATGGAAAAAGAAGAACATTTAAGAAAAAAATGCTTGATTAAATGTTTCTTCAAGCATA
ATTCTGCTGGTAATATTTCTTTCTTTTTAAATGCAACTTCTGTTTTGTTTTTATTCGTAAAAGACACAATTTCAT
ATTGCTTGACTACAGTACCAGCAGGCAGAGCATTACGTACAAATCAGGAGTAAAGCTTTCGTCAGTGTAGATGAT
CGTATCCTGAGCCATGTCTTCTTCTGAAGTGGCTCTCCAGAAGGCTGTCAGCTCGGATCTCATGTATCTACGCTG
ATTATAAATATGTTCATGACAGGGTGGCATCTGCTTCACAGTATTGACATCCACATCTATGTGTGTGGTGGGATA
TGGAGCATACATGACTTGCCGGAAAGGCAGCACAAAGCTTCCAGTTGAATCCTGTCAAAATAAAAGGAAAATTTA
CTGTCTTACATGCCAAACGATATGAATAATTGTTTTTTAATTTTAAAAAATGGTCTCTGATTTAGCAAATCCATC
TCTAAAAATTTATCCTAAGAAGCTATTCATGAGTGTGTGCAAAGTTTTAGTGACAGAAGTGTTCAGTATAGCATT
GGTTTCAATAGCAAAACATCAGAATGACCTAAGCATCCAAAGGAAGTGGTTAAGTAAATAAAAATGAATTTATTA
TTTGGCATCTATAGATGAAGTTGTTGAAGTTTGTGTTGATATTCAAAGAGGCTTAGAATCTGCTGTGAAAAAAGC
AGATTAGAACATGCAGAGATGATCCAGTGGGGGTGGGAGTCTTAAAGAAAATACATGCAAAAGGGAAAAAAAGAC
TAGAAAGAGATATACCAAATATTAACAGTGGGTATTCACTGGATTCAAAATTACAAGTGTCTTTTAATTTCTTAA
TTATTCTATGCACCTCAATGAGTATGTATTTTTATAAAACAAATTTACTTCTTTCCTAATCAGTTGGGGTAGCCA
GTTACCATGTAATAATAACCTACTGCGTATGAGGCACTGTGCTATGAACAGAACAGAAGAAAGAGGCTATTTCCA
TTTTTTTACTATTATAATGCTGTGATAATCATCTTTGTATAATGTCAAAGGTATTTTCTGGAAAACTATATGATT
TGTATTTGCATGGAGAACTATTAAATATTTTTTCATTAGATCATAGTTTTGAAATTTCCTCTAGGCATCTGGTAT
AGGTCCCTTTCAGAACTAAAATTATATAATTAAATGAGAGGGTGCTGAGGAAACAAAAGTATTAAGCACTTTATT
ATGCCTTTAACAGGAGAAGTTCTAATTTACAGATATGTATAACTCAAATAGAAACTACGTTTCCTTTAACTAGTT
TCTATATTTAAATACTATCATTCTGAGTTCATAGCAGCACATTATTGAAAATGCACAAAAGCCTAATATAACTTG
TATTTGCAACAATTTTTAAATTTTTTTATTTCATTTTGCCGTGGCTACATCCTCAAGAAAGTAGTCACTATGAGA
CAATCACATAACCATTAGGAAAGCTATTTCTTCACGTCTTGGGACTATAATTGAAATAACAATAATAGATTCAAC
AAGGTGAACAACTTTCTTCCCTTTAAGTATTATAAATAATTGCTAATTTACATTTCTCTGTTTTGCTCCTTGACA
GTAGACTCAAATAAAAGAAAAATACAATTTTTTTCTAATATATATTATATATAGAAATATATATATTTTAGTACA
CATACATAGGAATTGCTTAAATCCTATAAGCTTCTTAAAGATGTTTAAAATTTTTTTTCAATAAAATTCAAATCT
ATTTAGTTAAAAAGAAATGATCTTATTTTTGATGTGCAACCTGATTTAAGCATGTGTTAAAAAAAAAAAAGTCCT
GAGGCTAGACATGTAGGAACAGGGACCCACCTGGAACACAAAGGGTATTCTATGGTGTTTCACTGATGATACTAA
CTATAAATCCATAAGACATATAGTCTATGTGCAGAACTGTGTAAAGGAAGTCAGTCTCTGGGCATGTCAATATGA
GATACATTAAATGCTAATATTTAAGATTTGTCTATAAAGAGTCTCAAAAATGATTTTAGAAAAGTGGTTTCACTT
GTGATAACTAGAAACTATACCTTTAGCAGGCCTTGTACAAAGAGCCCTGACTCATATTTAAATGATGATTCTGCT
TCACATAACCTGGAGCATTTTCTCTCTGCTGGAGTCAGAAAAAGGCATAATGTTCTGACTATCTATAAAAGAAAA
TATTTTAGCATTAAAACATGAAGTAAAAAGACCACTGATTTGCTTATGAAAGATATCTGAAATTTTAATTGTTAT
TATCAATAAAACATATCCTAAGAAATAAGTATTCTTTAGTCACCTGGAATCCATGGGATGTAACAGTTGTTGCTT
AAAATAGGGATTCTGTGGTCAAATAAATTTAGAAAATGCTTGGTAAATTTACTGTATGGCATCTCAAAACCTTTA
ACATTTGGCTACATACTGTGACTCTTCAGGTGAATTATATCATCTGCAGACTTTCTCAGACTTATTTGACCATGC
AACTTTTATAGCTTCTGTTTAGTGGGTCTACACATGAAATTCGTTGTAAGAAATATCAAAGAATGTCCAGATCCT
CCAAAAAGAAGAGAATTAATTATTGAATTTGATTTAAAATAAGCAGGTCATTGGGTGGGATTTAGAAATCTGATT
CTAATAATATTTGACCTACAGCTCTATTAGGAAAAATAAAAAGGCATGTAACTATCTTGAAATTCAAACCATATC
CACAGTTATTATGTTAAAGGAGTGGTTTTCAACCTAGAGTGGTGGCAACTTCTTTCTCAAATTAAAACAAGATAA
AAGGTAAGCTATTCTCCCTCAAGAGGATGGAGGATGGGAAAAATGTTTAAAAAAAAAAAAAAAAACACTCCAACT
ATGGAGCCTTTCTCCCTTCATAAAGCAGCTCGGCAGTCACTCTGTGCAACCTAAGGCTTTGGAGATCACAGATGG
AAAGCCACCTGTTTGAGGTAACAGAAGGAATAAGGTCACTAGTTCGTAGATGCAATATAATGACACAGGTATACT
AAGCTCTCATAAATGGTTATATGAGAAATATAAATTAAGGCTCATGTAAATATACAAAGTAGCTGATTACAAAAA
AAAATTATGAATATCTTTGTAAAGTATCATTTCCAACATATTTCCTATGTAAAACTTTTTTTAAAAAATTAGGTT
TGCTGAAATTGAAAGATACACATACTTATCTCTGAACTCTTTCTAACTAATGGTCAGTGAAGAAAAGTGCAAAAT
CCTTTAGTTTATTAGCTAATGCTTGGAAATGTAACTGTTCATTAATCCTTAATTAACTCAAGTAGCACTGAAGGA
AAGGGTCAGAAACATTACTGAATAAAGTATAATAATCAATGACCACTTAATCCCAATAGCTCCCTAGAAGGGACA
GATTTAGAAGGAAAGCGAAGACAATGAAATCAAGATGAATAAACAAATAACATTTCTTTGGAACTACTACCAAAA
GTACATGACTATCTTCAGATTTGTTAAAGATAACATTGGGAAATAGAAGAGTAATTTTTTTTATATATCTGATTT
TAATATATTCTCAAAACCATTTATACACTACTGACACTGGTATTTCCGAGCTATCAAAATAAACTGATAAATGAT
TCTTACTCAGTTTATTTCAAACTCACTGTTGCCACAAGGTGTCTTAGCAATTTGATGAGATTACATTGCCTCCTT
ATACTACTAGATCATTTTAATTGCAACCTACCATTTAAATGACAATCCATGATATATCATCAGTCTTAAAGAGTC
AAATCATTTGCTAGATTATAAAATAAACTACCTTATTTACTTTCTCTGCACTGCTACCTACTACAACGGAACAGC
CACAGGTTTGCAAGTGTGAGCTGATGGCACTGTAAGTTAAAGAAAACAGATTAAAAACATTGCCTATAAAACAAT
TTAACAAACTAAAAACAAAAAAAAGTAGGTGAGCTCTTCAAATAACTCAGAATAGCTTTATATGATAAACACCGA
AGCTATAAGCACAATGTTATCTTTTATTTGTATAGGAACCTACATTTTCTAGAGACCTTTCACAGAAATTTTCTT
ATTGAGCCTTAAAACAGCCCAATTAGTCAGTATAATATCATTTAATTAATGTATTTATTTATTGAAATACCATCA
TTTTATAGCTGAAGAAATTGACATGTAGAGAGATTAAGTGACTTACTTAAAGTCAAATGGGATTTAAAATGATGT
ATGAAAGGCTGACACTGAACAGATACAGGACTAAAGTGCTTCTGATTCAAGCCATTAAGGCTCTTAGGTTAAACA
CACTCATGCCTCTGATACTCCATCATGAGCCTAAAGGAAAAGACTGTGAACATAAAAGTGAATACTTTATACTTT
TACTTCTCTTTTATTAAAAGTAAAATTTCATGAAAATCTGTAACTGTGAAGAAACTTTAAAACAGAATATAAGAT
AATACATGTAAAGCAACTAGTAAAGGAACTAACATGTAGGCACTCAACAAATACTGGCTATTTCTAGAAGAAATG
TAAATAGGAAATGTTAGCTATGAGCTATTATTAAGTGTTTTTATGTTCCAGGCACTGTTCTAAGTGCTTTATATT
ATTTATCTTACTCAATGCTTATAACAACCCTACACATTAGGTACTATTACTATTATTGCCATTTTACAGATGAGG
AAATAGGTGTATAGAGAATTCAGGCACCTTGCCCACGGGTACACAGCATTAATCCAGGGAGTCTGGTTTAAGGGC
ACAAACTCTTAAGTACTAAACTCCACTGCTGGATGGAAAAAGATCAGTATAAATATGAATAATTTTGTTCTACGC
CTAAATAACTTAAGTTCATCTACAGTACAACTTAATATGAAAGGATTCTGTTAGCTTTAATGAGAAGTAAAACAA
GAAACCAGAATCAAGCAAGGGGCCATGATTTCTTGTCTGGGATGGAAACTCGGTTTCTTTAAATAGCAAATGGAA
TAACACCAAATATATATAGAAATATAATGAGTGAAAAATAACACAAATTTAAGCAACAGTTCAAATACGTAATGT
CCCTAGAACAATCTAAGTAGACAGTCTGTTATTTTCTTTCTTCCAAATCTTGTCATAGGTGAGCATAAGATGGTA
TCTGCTTCATCCAGCTTTTATGAAAAGAAAAATTCTTACTTGAGAAGAAAGCCTTCATGACAGCTGTCACCAATA
TCATCATCATTGAGTACTGTATCAGCTATCTAAAATGCATCAAAAAATAAAAAAATTAGTCTGGCTGTAACATAG
TGTTGAAATAACACTTTTAATATACAAGTTTTCGGAAGTCTGGATTCAATATAACACACTGCCTTCATTTCCGAG
AATCAAGACTCCCCAAAAAACAATCTCTGTGCACTACCATAAACTTCAGAAGAACAAATGTGAAAGCTGGTCAAG
CAGGTTAAACAATTTTTACAAGAACAACTTCCTCTTCTGAGCTGTCAGAATCAGGAGACTAACCTAAATGACAAA
ATCAGAAAACAACAAGAATAGTTTCCTAAAGGTATCTCTTAACACTCATAGTGTGTGATTCAAAACGTCCTCAAC
AAATGATTAAGGAAACTAAATTTGTGACTACAAGTAAACTTCCATTAATGGTTACTACTTTGGCACACAGTTTTG
TTTCAAAAGACACTACATTAAATATTAATTGCTCCTATAAGAGCTGGGATCCTCCCACTTTTAGGAATTATAAAA
GTAATGAAATAAACAAAATGAATTTAATTTTGTCATCACTGATCAAAAATGCCTCTGTTTTGCCATAAAATCCAG
GATTTTGTGTGTGCTTATTTGCTAAAGTGGCTAATACTGTATGTGAATAGTATGTATGACAAAGTCCTTACTATT
AAAATTAGAATATTAATAATATACATAATAATACTATAACCCCAAAAAACTCATAAAGTGTATAATTGCTCTCAT
TTAAACTTACATCTATTTCTTCAGGAACACTGTGTGATTTCATAGATGAAAGCAGTTCCATTACAGGAATCACTT
CTCCAGTAAGCATTGGAATAATACTCTGACCCTGCACAATAAAGTGACATGAAGTGAAGAAAATCACGTAATATG
AGAGAAGCTGGGCAATAAAAAATAAAAATAACATCAAACAATAACATTCTTTGATGAAAATACTTCGTAATTTGT
TCAAACACAGTATCAAACAAGTCTACTACATGTCTAAAGGATTTATATGCAATCCAAAGCTCACTTTTATTCTTT
CTTTTCTTTTTTTTTTTTTGAGATGGAGTTTTGCTTTCATTGCTCAGGCTGGAGTGCAATGGCGTGATCTCAGCT
CACTGCAACCTCTCCTCCCAGGTTCAAGTGACTCTCCTGTCTCATCCTCCCAAGTAGCTGGGACTATAGGTGCCG
CCACCATGCCCGGCTGATTTTTGTATTTTTAGTAGAGACGGGGTTTCGCCATGTTGGCCAGGTTGGTGTCAAATT
TCTGACCATGCCCGGCCCTAAAGCTCACTTTTATTCTTTAGAGAGTATGGAATCATTGGTTTATCGTTTACTGTT
ACATGCAATGATTAAGTCATCATGCCTCTTTTAGAAAAGATCTCCTTTAAAATTTGAGATAAAAAAAATTTGTTA
AAGGTCATCAATATATTTCATATTTAAAAATGAGGAAAACCAAGCACAAAAAGACTTTGAAATCCTTACCAAATA
GGTAAGGAAAACTTGAATCAATACCTAACCTCCATACTCATAAAAGTATAATCTACCCAAATGCAAATCAAAATC
AGCACATATATTTTAAGAATCAATAAAACAGAAAAATTCCCTTTAGAGCTATTTCAAGATATTACTACTTATTAC
ATCTTGAAATTGTAATTTTGAAATTTGTAGTCTATAGAATCAAACTGAAAATTCAGTATAACACATCACAAATGT
AAAGTGTCTCAAATATGGATGGTCCCTCATTTATTCACTACCACCACCAGTCTCATCAGTTTTGTGACCAACTTG
GTTAAGTAAATTTTTTGAGATATAAATGAAATTGTCAAATGACTATGCATTTTTAGCTAAACATATTTTTTAAAT
CATACATTATTTAAGATGAATTTAATACTAGTTGTTTTTCCTCACTTATTTTATGAAATGATTTTACTCAAAGTC
TTCATAAGCATCTTTAAGTTAGAATCTTTGTCAGACCCAGGGCCATTTTTGGAGTAACCTTAACCAGTTTTTCAG
AGCCCCATATTATTAAGTTGCTTGAGAATTTAAATGTGATGCTACTTCTGGAAGTTTTATCCTAAGCCATATGCC
CATTTGCATAATGCTGAAAGTTTTATTTAAAAAAAAACCATCCTTTAGTAACCTCCACAACTAACTATTCACTGT
TTTTAGTTTTTAAAGTAATAATTATCATGCCTGTTTACAATTACAATTCACACATTCAATCTAACAAGAATAATG
ACTAGATCCGTGTTAAATTTCCTTCCCTGTGAAGCAATTTTATCAGATGACAGCTACAACTGAAGTTGTTTCAAA
CTAATGCATCATCCCCAAACAGTATTGTTCAAAATAAAGTCGTTGTGAGATTTGCAAGAACTCAATCAAAAGGCA
ACTCCTCCTTTTCGGGAAGAATAATTTTGGGAAAATATTTCCTCTTAGGTTTAAGCATACATAGTATTTCATTCA
CAGTATCTCAGACATTATCAGTATAAGTGAATGAATAGCCTCACTGAAGCTCAACAACACCAAAAAAAAAAAAAA
AAAAAAAAATCCTACAGGGCTAAATACAGAAGAGGCTCTAAAAGAAAATCTCTTAAGTTTCTATTCCTCCTTGTA
CTTCCCAAACTTGAACTTCTCAGCAGTAAGATAACATTTTTAAGAAGAGCACTTAAAAGAGAGACCAAAATTCAT
TAATAGTAGTCAACTTAAGTAAAGGTTTCTGGTTTGAAAAAACAAAATCCCAGTAAAAGCAGAATTTTAGTTGGT
TCTAAGTTTCCTCAACTTGCGATAAGTTTACTTAATTAGTCTACTAATAACTAGTGGGTTAGAGGGTGCTGAAAG
TTACCCCATTCCTGGGGACCCTGCTTATTGACCAGCAAATAAGGACTGGGATTCTTTGGGTAAAGGGAAATCTTT
TCTTGTTAAGTCAGACCTTTACACAGAATAACTGTCTCTGAATTGGAAAGCTATCTACAAAAGTACAAACATAAC
AATTTGGTAAAGGAGATCATTGTATTGGGTTCTGTATTATGGCCATGTATTTTCACAAGTTTTTTTTTTTAATTA
CTTTTTTAAAGTATCATCTGTCTCATTCATGCTAAAAAGAAGCAAAGAAAGGCAAAACAGCCATGTTTAAAATAT
TGGAGTTTTACAAGGAGCATTGAGGGTCACCCACAAGAGGAAATGGAAGTAAAAGTGAAGAACTCTTTCTTCACT
GGAGATTCTCCTTCAAAAGAACTTCTCTGCTTTACAGTGAAATAGTCTGTACTTAGTTTCCGCAGGGGAAGCCAC
ACCCTTGTAACCATGCTTCTCAAACTCTTAGTGTCTGTTCCTGAGGGGCATTCAAAGCCAAGGGATAAACATGGC
ACATTTTCCTAGAGGAGAGGGTAAGAAATATCACTGACAAATTTTAATACTAAAATAGTTATGGAATAAAATGTA
AATTGCATGAGTCTTAACGATACAACATAAGACTTAGAAGAAATATTGTGTGGACCTGGGCCTACACCCCAGACA
GATACCTCAGGGGTACATATGCTCTCCTTCTGTTACAGCTACTTCTAGGGAAAGGTTCGAGAAGTAGTACCTTAA
AGAACATATCAGAGACAATTTTTTTTATTTTTACTATGAACAAGTTATCCAAAATTTATTCTGGGCAAACAGAAA
AAAAAAGGGAGCAAATATTAATTTGTAGATGCAATTACTATTTTCCTTTGTTTACTGATTTAACTCTTTGGGTTT
AAGATATGGAAATCTTCCTCCAGTTTATTCTGTACACCTCCATAAAAGCTCCATTAAAGGCTTATTCGTATGTCT
CCAAGGCCTTGACAAATGTAGCCATCAACCTTATACAGATACATGCTGTGAGAAAAACATTTGACAGTATGCAAT
TTGCATATACCTGATCTTCCATTCTCTCTGTGCCTTCTAAGATAATCTTCTGGACATTTTCTTGTCTTTCCTGAG
CAAGAGAAAATTTATTTAAAAAAACAACCCACAACATTTTGATACTTGCTTATTTTTCAATAGACATGTTCTTGT
GTAGTAATTTAGTTCACAAGAAAAATACTTTCTACTTTAGGGAAAAAATGGGGGCAGGGGTAGGAAATTAACCCA
ACAAATGCATGTTCTCATAAACAATACAAAATAAAATCAAAACAACCTTTATTCTGCAGTGAAAAAAAGATAACT
TCACAGAAAACAGTCAATGTAACATCTGCATAGTTTCAAAAAGGAAAAGAATGACTTGCACTTTTCAAATTAAAC
ATTATGATGTTGTTTAAAAGATTCTCCTGATTTTAAGAGTTTCATAATGTGAGAAAAAAGGAAGTAAGCCTGCAA
ACATAGTAAAAAATTATTCTTTTAAAAGATATTATTTTTCCTTACTATTGGGCAAAAGCCTTTTAAAACTGGTAA
TGCTTAATGGACTTTCAGGTTAGTATCAAACTGGAACACAGGAAGGAGAATTCAATGTGTTCTTTAGATACATCA
AAACTATACTGAAATGTAAATAGCATTATATATTCAACTACAGGATTTAGGAAAACAATAATTTCTGTAAGATTA
AAAGGAATTCTCTTGGGAACCATTCCATTCAACCTCCTCATTTTATGAACCTGGGAACTTGGCAAAGAGGTTAAA
GAGACCAAAGGCTACATGACCAACAGCTTATGAAACTATTACTTTGAACTGTTATACTTACACATAGTAGTAAGC
AAAAGACAGAATTGTGCAATGAAAGGGAAACAAAAGGTATTAGAGTCAAAGGCTCCCAAGAAGAATCCAGGGTCT
AAAAGTTTCTTTATTTGTCTAAGCTTTAGCTTTTCATCTATAATGTGGAGCTACCATTTCGTACCTTCCACAGTT
AATATGAAGATGACAGGTATCAGACCAGATGTATTTGTATCTAATAGGGTAAATGCAAAATAAATAACATTTATT
GTTTGATGTTCACTGCATATAATTAAAAAAATAAGATTTATATGTACCAGAAAATAAGCTTTCAACAGATAGGTT
AACATGATTAATAAGCTGAAAAATCACTTACCTTATGCATCCATATTCTTCCTTTCCGGATTATATGTGTTAATC
TATCAACACACACTCTATGAAGTGGGAGGTAGAAACTAAGTTCTGTCTGTGGAAGTATAATTGATAGTCCATATG
TGCTGCGATCCCCATTCCAGTTTCCATCAAAGATTAATGAAACAATAATCACTCCCTTTTCAGACAAGACAAAAA
ACTTTACATCTATAGCACCACTCTCTGCATTTCGAAGGATTTCTCCATTTAGAGTGTGGTTGGCAAGAAAAGTTA
TTTCTCCATCACTGAGAAGTACCTGTTCTGTCTTTGGAGCCCAAATGTGCCTTACTCTAGGACCAAGAATATTGT
CCCAGTAAGCAAAAGTAGCTGCTAATAAAGGTGATTTGCCACTTAAAGCAATCTCTGTCTTGGCAACAGCTGGAG
ATGGCGGTGGGCAAAGAGTCGACATCACTGCATTCCAACTGTCACATTATCCAAATGCTCCGGAGATATCTAAAC
AATGACATATGAAACCAATGATTAGGTTCAGCAATTTAAAGATATCCATCAAAACCCCAAATGATTTAGACATAT
TTGGTTTGTCCTCTTAAGTCAAAGATGTGGAATCCTGTTATCTCCTATCAGGATAAAGACATTCAACTAGCACAG
TAGGTGCACATTAAATGTTTGTTGATATGATCATTTTACAAGACATGGTAACTTGTTACTTATATTCAGGGCATA
CATTTAGAAATTCAAAGAAATAACTTAAAAAAGGGCTTCTTTACACTGATATTAAATGTTACATACTAAAGCTCA
TAGAATAGACCCGCAGTATTCCCAAATATCCAGTCCATGTGCAATTCTAGTATGACTGGAGATTTGGCCCCTAAC
CCATAGCAACTAAAAAGGAGAAAAACAGGAAGGGAAAGGCTCAGCTAGAGACTGACACTTGTGGGTTGAATTGTG
TCCCCCAAAAAGATATGTTCAATTCCTAACCCTTGGTGTACGTGAATGTGACCTTATCTAGAAATAAGTGTAATC
ATGTTAAAATGGGGTCATACTGGATTAGAGTGGGGCCTAATCCAATAACTGCTGTGTTTATAAGGAGAGAGATTT
GGAGACACAGAGACAAATGGTAGACAGCCATGTGAAGACAAAAGGCAGATACTGGATTGTTGAAACTACAAGGCA
AGAAAGGAACACTCAGGATTGCTGGTAACCACCAGAAGCCAGGAAGAGGCAAGGAAAGAGTCTTCTCTCTTGAAG
ATCATGCCCCTGTCAACACTTTGATTTCGGACTTCTAGCTTCTAGAATTGTGAGAGAATAAATTGCTGTTGTTTA
AAGCCACTCAGTTTGTGGTGCTTTGTTAAGTAATCTTAGAAAAGTAATACAACACCTAACAACAGAAATACTTTA
AAGCCGCTAAAAGGTCAAAAAAAAAAAAAAAAAAAAAGACATGGAAATACCACAAGTCTGGAGCCATAACAAAAA
ATGGGCAAACAGTCCTGTATCCTCAGTGAACTCTCTGGTTATGAGAATACTGAAGCCCGATCCTGATGTTTAAAA
CGACATTGAAGTATCAAGACAAAGATAAAAATATTTAATATGCTAGCCAAGAAACCAATACAGCATTTCATCACT
GCAAAGAGAGTTCTACACTAAATGGCTAGAATTTAAAAGCTTTAGTTATTTAGAACACGTAGAAAACAGAAGGGC
TAAATAGGGCCCGTTCAAGCCTTTGAATTTAATGAGAAAACAGACATGAGGAGAAGAACATAAACGCTCACATCC
AAGACAGAACCCAGGGCTCTTGGTCCCCTTGCTCAACTTGTACATCTTAATCCACATAAACATACCACTCTAAAA
AGGTACATCCTATGTGATATTAATGTAAAACAAATCATTCTTGCAAATACAGTTATGTGCCATGTAACGTTTCAG
TCAATGGTAGACTGCATATATGATGGTAGTCCCATTAGATTACAATGGACCTGAAAATATGCTATTGCCTTAGTG
ACACTGTAACCATCATAAGGTCTTAGTACTATTTTGCAAGTTATTTAAAGTATAGCACATACAATTATTACAGTG
TACAACACTTGATAATAAACTACTACATTGCTGGTTTATGTATTCACTATACTATGCCTTTTATTGTTATTTTAG
AGTGCACTCCTTCTACTTTTTTTTTTTTTAAGTTAAATGTAAAACAGCCTCAGGCAAGTCCTTCAGGAGGTATTC
AACAGAAAGCACTGTTATCATAGGTGACAGCTACATGTGTGTTATTGCCCCTAAAAACCTTCCAGTGGGACAAGA
TGTGGAGGTGGAAGGCAGTGAGGTGGAAGGGAGTGATACTGATGATCCTAATCCTGTCTATGCCTAGGTGAAAGT
GTGTGTGTTTTAGTTTTTAACAAAAACGACTAACAAGTAAAAAAAAAAATTTAAAATAGAAAATAGAAAAAAGCT
TCTAGAATAAGGATACAAAGAAAAAATATTTTTGTATAGCTATACAATGTATTTGTGTTTCAAGCTAAGTATTTT
AAAAGTTAAAAAATTAAAAAGTTTACAAAGTTAAAAAGTTATAATTTTTTATTGAAGAAAAACTGTTAAGATAAA
TTTGGTGTAGCTTCAGCGTACTGTGTTTATAGTCTACAGTGGTGTACAGTGTTCTAGGCCTTCACATTAATTCAC
CACTCACTCACTGACTCACCCAGAGCAACTTCTAGTCCTGCAAGCTCCATTCGTGGTAAGTGGCCTACACAGGTA
TACCATTATCTTTTATACCATACTTTTACTGTACCTTTTCTCTGTTTGCATATATTTAGATAAATATTTACCACT
GTGTTACAACTGTCTATAGTATTCAGTACAGTAACAGTTGTACAGGTTTGTGGCCTAGGAGCAACAGACTATACC
ATACGGCCTAGGTACATAAAGGCTATACTATCTAGGTTTGTGTAAGTACACTCTATGATGTTTGCATAATGACAA
AATCGCCTAATGATGCATTCCTAAGCAATGTGTGATTGTACTATAATTGAAGACTTGTTATCTAAGACTGAAAGT
AAAAAGAATTGCAATTTCACCTAAGCAAGTCTAAAACTGTGAAGTCTATTTATAATAATAGCAATACAAAGCAGC
TAATAGGCAAACTATGATATACCTATCTTTGCCATATGATTGCTTTGGGAGCTAACATTTGATCTGTAAATGTAT
GACAAAGTAAACAATTTTACTTAAAGAATTTCATCCACATCTTGTCAAGAGAGTTCAGTCTGATGGAAAGCACTG
ACTTCTATTTACAGAGCATTAGATGAGTGCTTTTATCATATTATGAGTAGGCATACAGAGCCTGGCAAAACAGTT
AACTCTAAGTATGTACAGAAATGGTTGAACACAACGACAGTTTTAACACGTGTATTTGTAATTTCAAAAATTCAT
TTAGGTAATATTTACTTTTAAATATGTTGTATCAATTTAATAGTCTTAAGAGACAGCACTAGATATAAGCCGTAC
AGCTTCTTTAAAATATCCACTGTTTTTAATACAATGTAAGCAGTCAGTTTACAATGATCAAATATAGGAATGTAA
TCTGAATTGAAATGGTAATGACACTACTGCTGTCATAACTAACAACAGCAAACTGGAGGCCAACATAATGAATTA
AGTTAACATACAACCATAAAATTATATTGCAAACATATTTTTCTTTCATTCTTTTAGGTTAAAAAGGTGGATAAT
CATAAAGGCAATATTACAACTCTAATATTTCATCATTAAACTGAAAATAAAAGTATTTCCTAAAACAGAACTGAA
CCCTGGAGCAAAATCTGATTGAATTATAGGGAAACTTTTACCACGTTGTGAAAATTGAACTATTATACTGCTAGT
TACACTCTCACTCCTAACAGAATAAGAAAAAAAAAATGGGCCGGGCATGGTGGGTCACACCTGTTATCCCAGCTC
TTTGGTAGGCCGAGGCAGGTGGATCACCTGAGGTCAGGAGCTCAAGACCAGCCTGGCCAACATGGTGAAACCCCA
CCTCTACTAAAAATACAAAAAATTAGCCGGGTGTGGTGGTGGACACCTGTAATCCCAGCTACTCGGGAGGCTGAG
GCAGGAGAATCTCTTGAACCCGGAGGTGGCAGAGGTTGCAATGAGCTGAGATGGCGCCACTGCACTCCAGCCTGG
GCGACAGAGAGAGACTCTGCCTCAAGAAAAAAACAAACAAACAAACAAACAAAAAGAATAAGAAAGAAAATGAAG
GACAAAGATCATACTGAATTGCTTAGTTTTAAATCCTACCAAAAGAAATAGCCTGGGAAATGAAATGTCACAGAG
AAGTATAATCAGGAGAGCTGTACAATTATTTTACTAATACTTGAAGTCATCGTCTTTGGTGAGAAAAATCCATAC
ATGCAAATGCAGCTGAAAAAAATCAGCTCAAAACCAATAGTTGTTTATGTACCTATCTTACGTACATGTAGTGCT
GTCTACTCCAGAGAGTTACCAAACATTAGCCAGTCTTTTGAGGGAAGCCAAGATTCAAATTGAGTGAGACGGTGG
CTTGCTCACAGGGTTCATGAGAGGTTTCCCAATACACTTTCTGGAAATAATCCCATACATGCAGACATGATTACA
TTAATTAACATCTGCTAAAACTGTTAGTAGAGTGCTAAGTTTGAGGTTTTGCTTTTTCTTTAAACGTCTGTTAAA
AAATCAACCATCTCTTCCCTGATTGGTATTTAGAAAGGTGGTTGGTCCACTGCTATTGTAGTGAAAATTCTACAA
TCATAAAGCCCTCACTTCTTGTTTTTTAGAGACAGGGTCTCGTTTTGTCATCCAGGCTGGAATGCACTGGCAGGA
TCATAGCTCTCGGTAACTTCAAACTCTTGGGCTCAAATGACCCTCCTGCCTCAGCCTCCCAAGTAGCTAGGACTA
CAGGTGCACATCACCACGCCCGGCTAAGTTTTTAATTTTTTGTAGAGACAGGGTCTACGTTGCCCAGGTTGAGCT
TGAACTCCTGGCTTCAAGTGATCCTCTTGCCTCCGCCTCCCAAAGCTCTGGCATTACAGGTGTAAGCCACCTTCT
CCAACCTGGCTCTCAATACTTGTAACCATGCTGTTTATTTTCTCCCAGCCCAAAGAGAAGCAGGATCCTAAACCG
TCCACTTTCCACAACAGGAGCTGCCCAGGACCACTTCAAGGACAGTGAACTGTTTACAGTACCAGAAAGTTCACA
ACACTTTCTCAATCTTCAACATCAGGGAAGACTGGAAGGTGAAGTTCATATCACTATCTGGCCATTTCTCACAGT
TCCAAGTTTCTCAGACAATAGGTAGGCTAACCTAGTCCTCCTGGGAACTATCTAATTAACGTAGAATAGAACCCG
AGGGCAGACTTGAAAAACAGAAGTCCTCCTTGGTTTACTTTGTTTCTCTGAAAGCAAATTGTGGAGTGCCAACAT
AGCCAAACAAAATATTTTATCAACTTCATAAGGTGCTTGTAATTTTTTCCTGGAGCAGGTAAATGCTGGCTTAGT
GAACAATCTGGAATGTGGTAATTACTCTCGTTCTTGTTTCAGATGTACTATCAGCATGTAGCAGTTTCCAACTGA
TTCAGGGTTTTCCTAAAGTGGCAGGCCTTGGCAGAGGTGGTGACAACAATGCCCGTGTCAAATGACACCGTATTT
CAAGTATTCTGACTCCAGGTTATTAATATCCCCTATATGATAGTCTTGTTTCTGTGATATTCACAGATTATGTTA
AAAGTTTCCCAAAGTCTGAGAAAAATCATATCTTAACAGTATCTTTTTTTTTTTTGATCCTTTGTACAAAAGTAG
AAGTAATGCCAGACAGATTACGTACCCTTGTTGTGAACAACTGGTGCATGGCAACTGTTTGAATAGAAATTTACC
AACTGCCACAACCAGGCAACTACTCTCCCAGAGCCTAACAATCTCGATTGCATCTGAAAGGGCCACCCCTCCTGG
GAAAGTGCAGGACCTCCCTCCTGTTTCTGAATACAAAGCCTGGTGGTGTTCAACGCGGCCAGATAGACCCAATGA
GCACACGGACATGTAATCTGTGCACTTCTTTAGACAACTGATTACCATCAGTCAAGTGATGCCCAAGTCACAATA
GTCACTTCCTTTAAGCAAGTCTGTGTCATCTCGGAGCTGTGAAGCAACCAGGTCATGTCCCACAGAATGGGGAGC
ACACCGACTTGCATTGCTGCCCTCATATGCAAGTCATCACCACTCTCTAGAAGCTTGGGCTGAAATTGTGCAGGC
GTCTCCACACCCCCATCTCATCCCGCATGATCTCCTCGCCGGCAGGGACCGTCTCGGGTTCCTAGCGAACCCCGA
CTTGGTCCGCAGAAGCCGCGCGCCGCCCACCCTCCGGCCTTCCCCCAGGCGAGGCCTCTCAGTACCCGAGGCTCC
CTTTTCTCGAGCCCGCAGCGGCAGCGCTCCCAGCGGGTCCCCGGGAAGGAGACAGCTCGGGTACTGAGGGCGGGA
AAGCAAGGAAGAGGCCAGATCCCCATCCCTTGTCCCTGCGCCGCCGCCGCCGCCGCCGCCGCCGGGAAGCCCGGG
GCCCGGATGCAGGCAATTCCACCAGTCGCTAGAGGCGAAAGCCCGACACCCAGCTTCGGTCAGAGAAATGAGAGG
GAAAGTAAAAATGCGTCGAGCTCTGAGGAGAGCCCCCGCTTCTACCCGCGCCTCTTCCCGGCAGCCGAACCCCAA
ACAGCCACCCGCCAGGATGCCGCCTCCTCACTCACCCACTCGCCACCGCCTGCGCCTCCGCCGCCGCGGGCGCAG
GCACCGCAACCGCAGCCCCGCCCCGGGCCCGCCCCCGGGCCCGCCCCGACCACGCCCCGGCCCCGGCCCCGGCCC
CTAGCGCGCGACTCCTGAGTTCCAGAGCTTGCTACAGGCTGCGGTTGTTTCCCTCCTTGTTTTCTTCTGGTTAAT
CTTTATCAGGTCTTTTCTTGTTCACCCTCAGCGAGTACTGTGAGAGCAAGTAGTGGGGAGAGAGGGTGGGAAAAA
CAAAAACACACACCTCCTAAACCCACACCTGCTCTTGCTAGACCCCGCCCCCAAAAGAGAAGCAACCGGGCAGCA
GGGACGGCTGACACACCAAGCGTCATCTTTTACGTGGGCGGAACTTGTCGCTGTTTGACGCACCTCTCTTTCCTA
GCGGGACACCGTAGGTTACGT
SEQ ID NO: 15
>NG_031977.2 Homo sapiens C9orf72-SMCR8 complex subunit (C9orf72) ,
RefSeqGene (LRG_658) on chromosome 9
TTGTAAGTTCTCTGAGGCATCCCCAGAAGCTGATGCTGCCATGCTTCCTATACAGCCTGCAGAACCATGAGTCAA
TTAAACCTCTTTTCTTTGTAAATTACCCAGTCTCAAGTATTTCTTTATAGCAATGCAAGAATGGACTAATACAGA
AAATTGTTACTGAGAAGAAGGGCATTGCTATAAAGATACCTGAAAATGTAGAAGTGACTTTGGAACCGGCTAACA
GGCAGAAGTTGAAACATTTTAGAGGGCTCAGAAGAAGACAGAAAGATGAGAGAAAGTTTGGAACTCGCTAGGAAC
TTGTTGAGTGGTTGTAACCAAAATACTGATAGTGATATAGACAGTGAAGTCCAGGCTGAGGAGGTCTCAGATGGA
AATGAGAAATTTATTGGGAATGAGTAAAGGTCAGGTTTGCTATGCTTTAGCAAAGAGCTTAGCTGCATTGTTCCT
CTGTTCTAGGGATCTGTGAAATCTTAGACTTAAGAATGATGATTTAGGGTATCTGGCAGAAGAAATTTCTAAGCA
GCAGAGTGTTCAAGAAGTAACCTAGCTGCTTCTAATAGCCTATGCTCATAGGCATGAGCACAGAAATGACCTGAA
ATTGGAACTTACACTTAAAAGGGAAGCAGAGCATAAAAGTTTGTAAATTTTGCAGCCTGGCCATGTGGTAGTAAA
GAAAAGCTCGTTCTCAGGAGAGGAAGTCAAGCAGGCTGCATAAATTTGCATAACTAAAAGGAAGGCAAGGGCTGA
TAACCAAAACAATGGGGAGAAAGACTCATAGGACTAACAGGCATTTTATTTTATTTTATTTTTATTTTATTATTA
TTATACTTTAAGTTTTAGGGTACATGTGCACAATGTGCAGGTTAGTTGCATATGTATACATGTGCCATGCTGGTG
TGCTGCACCCATTAACTCGTCATTTAGCATTAGGTATATCTCCTAATGCTATCCCTCCCCCCTCCCCCACCCCAC
AACAGTCCCCAGAGTGTGATGTTCCCCTTCCTGTGTCCATGTGTTCTCATTGTTCAATTCCCACCTATGAGTGAG
AACATGTGGTGTTTGGTTTTTTGACCTTGCAATAGTTTACTGAGAATGACGATTTCCAATTTCATCCATGTCCCT
ACAAAGGACATGAACTCATCATTTTTTATGGCTGCATAGTATTCCATGGTGTATATGTGCCACATTTTCTTAATC
CAGTCTATCACTGTTGGACATTTGGGTTGGTTCCAAGTCTTTGCTATTGTGAATAGTGCCACAATAAACATAGTG
TGCATGTGTCTTTATAGCAGCAGGATTTATAGTCCTTTGGGTATATACCCAGTGATGGGATGGCTGGGTCAAATG
GTATTTCTAGTTCTAGATCCCTGAGGAATCGCCACACTGACTTCCACAATGGTTGAACTAGTTTACAGTCCCACC
AACAGTGTAAAAGTGTTCCTAATAGGCATTTTAGGCTTTCATGGTGGTCCCTCTCATCACAGGCCCCGAGGCCTA
GGAGGACTGAATCATTTCCTGGGCCAGGCCTAGGGCCCCTGCTCCCTCTTACAGCCTTGGGACTCTGCTCCCTGA
ATCCCAGCTGCTCAAAGGGGCCCAGGTACTGTTACAGTAGGTAGCTAATCAGGCATGAGTGGGGTAAGAGAGAAG
TCCCCACCACCCACCAGGAATGTCAGGCAACCATCAGATGATGGTCAGGCAGTTGTCATACTGCCTCTCTAAAAT
AGTAATTGGTTGCAGCCAGCACCAGGGAGAGGCAACTTCTCAATAGATAGAAACACCTGAAATTGGTAACTGGGC
GCTTCCAATAAGATCTCAGGAACTGAGAGAGTGGGCTTAACATGCACATTAAGAGGCAAAATGGTGAAGTATGAC
CTTTGGGGGCATTCCACCGGAAAAGGGAAGAAAGCCTCAGGTAAGCATGTATACAACTCCAGTAAACACACTGCA
CACGCTCACCTTCCAAGTGCAAGCAGGGCACCATGCATGCGGCAAGCTCACCCTTAGGGAAGGACCAAGGGAAAG
GGGCACAAGATGTCAGAAGTAGGCCAGTGTATAAGATCCTAGGTTCAAGGTCAAACAGGGCACTTGACCTCCAAG
GTGCCCACTTGGGCCTCTTCCAAATGTACTTTCCTTTCATTCCTGTTCTAAAGCTTTTTAATAAACTTTTACTCC
TGCTCTGAAACTTGTCGCAGTCTCTTTTTCTGCCTTATGCCTCTTGGTCAAATTCTTTCTTCTGAGGAGGCAAGA
ATTGAGGTTGCTGCAGACCCACATGGATTTGCAGCTGGTAACTCAGATAACTTTCACCAGTAAGAATACAGTTCA
GGCTGCTGCTTCACAGGGTGCCAGGCATAAGCCTTGGTGGCTTCCATAAGCTGTGAAGCCGGCGGGCGCACATAA
TGCAAGAGTTGAGGCTTAAGAAGCTCTGCCTAGATTTTAGAGGATGTATGAAAAAGCCTGGATGTCCAGACAGAA
GCCTGTTACTGGGGTGGAATCCTCATGGAGAACATCTACTAGGGAAGCAAGGAGAAGAAATGTGGGGTTGCAGCC
CCCACAGAGAGTCCCCTGGGGCACTGCCTAGCAGAGCTATGACAAGACAGCCACCGTCCTCCAGACCCCAGAATG
GTAGATCCACCAACAACTTGCACCCTGCAGCCTGGAAAAGCTGCAAGCACTCAATGCTAGCCCATGAGAGCAGCT
GTGGGAGATGAACCCTGGAAAACCACAGGGGTGGTTCTGCCCAAGGTTTTGGGAGCCCACTCATTGCATCAGTGT
TCCCTGGGTGTGAGTCAAAGGAGATTATTTCAGAGCTTTAACATTTAATGACTGCCCGGCTGGCTTTCAGACTTG
CAATGGGGCCCTATAGCCTCTTTCTTTTGGCAGATTTCTCCCTTTCGGAATGGCAGTATCTGCCCAATGCCTATA
CCCCCATTGTATCTTTGAAGCAATTACCTTGTTTTTGATTTTACAGGTTCATAGGTAGAAGGGACTAGCTTCGTC
TCAGGTGAGACTTGGGACTTTGGACTTTTGAATGAATGCTGGATCGAGTTAAGACTTTGGGGAACTGTTGGTAAG
GCACGACAGTATTTTGCAATATGAGAAGGACATTAGATTTGGGAGGGGCCAGAGTTGGAATAACATGGTTTGGAT
CTCTGTCCCCACCCAAATCTCATGTTCAACTGTAATCCCCAGTGTTGGAGGTTGGGCCTGGTGGGAGGTGAGTGG
ATTATGGGGTGGCTTCTAATGGTTTTGTACAGTCCCCTCTTGGTACTATATAGTGAGTTCTGACAAGATCTAGTT
GTTTAAACGTATGTAGCACCTCCCATTTCTCTCTTCCCCCAGTTCCTGCCATGTGAAGTCTGGGGTCTCCCTATG
CCTTCCATCATGATTTTAAGTTCCCTATGGCCTGCCCAGAAGCTGATCCAGCCATGCTTCTTGTACAGCCTGCAG
AACTGTGAGCCATTAAACTTTTCTTTATAAATTACCCAGTTTCAGTTATTTCTTTATAGCAGTGTAAGAATGGAC
TAACACAATTATTAACGCTAGTCCTCATGTTGTACATTAAATCTCTAGATGTATTAGACGTAACTGCAACTTTGT
ACCCTACCCTACAATTTTCTTTCCCCCCAAGCCCCCCAACCAAGGGTCTACTCTGTTTCTATAAATTCAGTTGTT
TTTTAATTCCACGTATAAGTGAAGTACAACTCAGTGTAGAAACTTGGTAAATGCTAGCTACTTGTTATAAGCTGT
CAGTCAAAATAAAAATACAGAGATGAATCTCTAAATTAAGTGATTTATTTGGGAAGAAAGAATTGCAATTAGGGC
ATACATGTAGATCAGATGGTCTTCGGTATATCCACACAACAAAGAAAAGGGGGAGGTTTTGTTAAAAAAGAGAAA
TGTTACATAGTGCTCTTTGAGAAAATTCATTGGCACTATTAAGGATCTGAGGAGCTGGTGAGTTTCAACTGGTGA
GTGATGGTGGTAGATAAAATTAGAGCTGCAGCAGGTCATTTTAGCAACTATTAGATAAAACTGGTCTCAGGTCAC
AACGGGCAGTTGCAGCAGCTGGACTTGGAGAGAATTACACTGTGGGAGCAGTGTCATTTGTCCTAAGTGCTTTTC
TACCCCCTACCCCCACTATTTTAGTTGGGTATAAAAAGAATGACCCAATTTGTATGATCAACTTTCACAAAGCAT
AGAACAGTAGGAAAAGGGTCTGTTTCTGCAGAAGGTGTAGACGTTGAGAGCCATTTTGTGTATTTATTCCTCCCT
TTCTTCCTCGGTGAATGATTAAAACGTTCTGTGTGATTTTTAGTGATGAAAAAGATTAAATGCTACTCACTGTAG
TAAGTGCCATCTCACACTTGCAGATCAAAAGGCACACAGTTTAAAAAACCTTTGTTTTTTTACACATCTGAGTGG
TGTAAATGCTACTCATCTGTAGTAAGTGGAATCTATACACCTGCAGACCAAAAGACGCAAGGTTTCAAAAATCTT
TGTGTTTTTTACACATCAAACAGAATGGTACGTTTTTCAAAAGTTAAAAAAAAACAACTCATCCACATATTGCAA
CTAGCAAAAATGACATTCCCCAGTGTGAAAATCATGCTTGAGAGAATTCTTACATGTAAAGGCAAAATTGCGATG
ACTTTGCAGGGGACCGTGGGATTCCCGCCCGCAGTGCCGGAGCTGTCCCCTACCAGGGTTTGCAGTGGAGTTTTG
AATGCACTTAACAGTGTCTTACGGTAAAAACAAAATTTCATCCACCAATTATGTGTTGAGCGCCCACTGCCTACC
AAGCACAAACAAAACCATTCAAAACCACGAAATCGTCTTCACTTTCTCCAGATCCAGCAGCCTCCCCTATTAAGG
TTCGCACACGCTATTGCGCCAACGCTCCTCCAGAGCGGGTCTTAAGATAAAAGAACAGGACAAGTTGCCCCGCCC
CATTTCGCTAGCCTCGTGAGAAAACGTCATCGCACATAGAAAACAGACAGACGTAACCTACGGTGTCCCGCTAGG
AAAGAGAGGTGCGTCAAACAGCGACAAGTTCCGCCCACGTAAAAGATGACGCTTGGTGTGTCAGCCGTCCCTGCT
GCCCGGTTGCTTCTCTTTTGGGGGCGGGGTCTAGCAAGAGCAGGTGTGGGTTTAGGAGGTGTGTGTTTTTGTTTT
TCCCACCCTCTCTCCCCACTACTTGCTCTCACAGTACTCGCTGAGGGTGAACAAGAAAAGACCTGATAAAGATTA
ACCAGAAGAAAACAAGGAGGGAAACAACCGCAGCCTGTAGCAAGCTCTGGAACTCAGGAGTCGCGCGCTAGGGGC
CGGGGCCGGGGCCGGGGCGTGGTCGGGGCGGGCCCGGGGGCGGGCCCGGGGCGGGGCTGCGGTTGCGGTGCCTGC
GCCCGCGGCGGCGGAGGCGCAGGCGGTGGCGAGTGGGTGAGTGAGGAGGCGGCATCCTGGCGGGTGGCTGTTTGG
GGTTCGGCTGCCGGGAAGAGGCGCGGGTAGAAGCGGGGGCTCTCCTCAGAGCTCGACGCATTTTTACTTTCCCTC
TCATTTCTCTGACCGAAGCTGGGTGTCGGGCTTTCGCCTCTAGCGACTGGTGGAATTGCCTGCATCCGGGCCCCG
GGCTTCCCGGCGGCGGCGGCGGCGGCGGCGGCGCAGGGACAAGGGATGGGGATCTGGCCTCTTCCTTGCTTTCCC
GCCCTCAGTACCCGAGCTGTCTCCTTCCCGGGGACCCGCTGGGAGCGCTGCCGCTGCGGGCTCGAGAAAAGGGAG
CCTCGGGTACTGAGAGGCCTCGCCTGGGGGAAGGCCGGAGGGTGGGCGGCGCGCGGCTTCTGCGGACCAAGTCGG
GGTTCGCTAGGAACCCGAGACGGTCCCTGCCGGCGAGGAGATCATGCGGGATGAGATGGGGGTGTGGAGACGCCT
GCACAATTTCAGCCCAAGCTTCTAGAGAGTGGTGATGACTTGCATATGAGGGCAGCAATGCAAGTCGGTGTGCTC
CCCATTCTGTGGGACATGACCTGGTTGCTTCACAGCTCCGAGATGACACAGACTTGCTTAAAGGAAGTGACTATT
GTGACTTGGGCATCACTTGACTGATGGTAATCAGTTGTCTAAAGAAGTGCACAGATTACATGTCCGTGTGCTCAT
TGGGTCTATCTGGCCGCGTTGAACACCACCAGGCTTTGTATTCAGAAACAGGAGGGAGGTCCTGCACTTTCCCAG
GAGGGGTGGCCCTTTCAGATGCAATCGAGATTGTTAGGCTCTGGGAGAGTAGTTGCCTGGTTGTGGCAGTTGGTA
AATTTCTATTCAAACAGTTGCCATGCACCAGTTGTTCACAACAAGGGTACGTAATCTGTCTGGCATTACTTCTAC
TTTTGTACAAAGGATCAAAAAAAAAAAAGATACTGTTAAGATATGATTTTTCTCAGACTTTGGGAAACTTTTAAC
ATAATCTGTGAATATCACAGAAACAAGACTATCATATAGGGGATATTAATAACCTGGAGTCAGAATACTTGAAAT
ACGGTGTCATTTGACACGGGCATTGTTGTCACCACCTCTGCCAAGGCCTGCCACTTTAGGAAAACCCTGAATCAG
TTGGAAACTGCTACATGCTGATAGTACATCTGAAACAAGAACGAGAGTAATTACCACATTCCAGATTGTTCACTA
AGCCAGCATTTACCTGCTCCAGGAAAAAATTACAAGCACCTTATGAAGTTGATAAAATATTTTGTTTGGCTATGT
TGGCACTCCACAATTTGCTTTCAGAGAAACAAAGTAAACCAAGGAGGACTTCTGTTTTTCAAGTCTGCCCTCGGG
TTCTATTCTACGTTAATTAGATAGTTCCCAGGAGGACTAGGTTAGCCTACCTATTGTCTGAGAAACTTGGAACTG
TGAGAAATGGCCAGATAGTGATATGAACTTCACCTTCCAGTCTTCCCTGATGTTGAAGATTGAGAAAGTGTTGTG
AACTTTCTGGTACTGTAAACAGTTCACTGTCCTTGAAGTGGTCCTGGGCAGCTCCTGTTGTGGAAAGTGGACGGT
TTAGGATCCTGCTTCTCTTTGGGCTGGGAGAAAATAAACAGCATGGTTACAAGTATTGAGAGCCAGGTTGGAGAA
GGTGGCTTACACCTGTAATGCCAGAGCTTTGGGAGGCGGAGGCAAGAGGATCACTTGAAGCCAGGAGTTCAAGCT
CAACCTGGGCAACGTAGACCCTGTCTCTACAAAAAATTAAAAACTTAGCCGGGCGTGGTGATGTGCACCTGTAGT
CCTAGCTACTTGGGAGGCTGAGGCAGGAGGGTCATTTGAGCCCAAGAGTTTGAAGTTACCGAGAGCTATGATCCT
GCCAGTGCATTCCAGCCTGGATGACAAAACGAGACCCTGTCTCTAAAAAACAAGAAGTGAGGGCTTTATGATTGT
AGAATTTTCACTACAATAGCAGTGGACCAACCACCTTTCTAAATACCAATCAGGGAAGAGATGGTTGATTTTTTA
ACAGACGTTTAAAGAAAAAGCAAAACCTCAAACTTAGCACTCTACTAACAGTTTTAGCAGATGTTAATTAATGTA
ATCATGTCTGCATGTATGGGATTATTTCCAGAAAGTGTATTGGGAAACCTCTCATGAACCCTGTGAGCAAGCCAC
CGTCTCACTCAATTTGAATCTTGGCTTCCCTCAAAAGACTGGCTAATGTTTGGTAACTCTCTGGAGTAGACAGCA
CTACATGTACGTAAGATAGGTACATAAACAACTATTGGTTTTGAGCTGATTTTTTTCAGCTGCATTTGCATGTAT
GGATTTTTCTCACCAAAGACGATGACTTCAAGTATTAGTAAAATAATTGTACAGCTCTCCTGATTATACTTCTCT
GTGACATTTCATTTCCCAGGCTATTTCTTTTGGTAGGATTTAAAACTAAGCAATTCAGTATGATCTTTGTCCTTC
ATTTTCTTTCTTATTCTTTTTGTTTGTTTGTTTGTTTGTTTTTTTCTTGAGGCAGAGTCTCTCTCTGTCGCCCAG
GCTGGAGTGCAGTGGCGCCATCTCAGCTCATTGCAACCTCTGCCACCTCCGGGTTCAAGAGATTCTCCTGCCTCA
GCCTCCCGAGTAGCTGGGATTACAGGTGTCCACCACCACACCCGGCTAATTTTTTGTATTTTTAGTAGAGGTGGG
GTTTCACCATGTTGGCCAGGCTGGTCTTGAGCTCCTGACCTCAGGTGATCCACCTGCCTCGGCCTACCAAAGAGC
TGGGATAACAGGTGTGACCCACCATGCCCGGCCCATTTTTTTTTTCTTATTCTGTTAGGAGTGAGAGTGTAACTA
GCAGTATAATAGTTCAATTTTCACAACGTGGTAAAAGTTTCCCTATAATTCAATCAGATTTTGCTCCAGGGTTCA
GTTCTGTTTTAGGAAATACTTTTATTTTCAGTTTAATGATGAAATATTAGAGTTGTAATATTGCCTTTATGATTA
TCCACCTTTTTAACCTAAAAGAATGAAAGAAAAATATGTTTGCAATATAATTTTATGGTTGTATGTTAACTTAAT
TCATTATGTTGGCCTCCAGTTTGCTGTTGTTAGTTATGACAGCAGTAGTGTCATTACCATTTCAATTCAGATTAC
ATTCCTATATTTGATCATTGTAAACTGACTGCTTACATTGTATTAAAAACAGTGGATATTTTAAAGAAGCTGTAC
GGCTTATATCTAGTGCTGTCTCTTAAGACTATTAAATTGATACAACATATTTAAAAGTAAATATTACCTAAATGA
ATTTTTGAAATTACAAATACACGTGTTAAAACTGTCGTTGTGTTCAACCATTTCTGTACATACTTAGAGTTAACT
GTTTTGCCAGGCTCTGTATGCCTACTCATAATATGATAAAAGCACTCATCTAATGCTCTGTAAATAGAAGTCAGT
GCTTTCCATCAGACTGAACTCTCTTGACAAGATGTGGATGAAATTCTTTAAGTAAAATTGTTTACTTTGTCATAC
ATTTACAGATCAAATGTTAGCTCCCAAAGCAATCATATGGCAAAGATAGGTATATCATAGTTTGCCTATTAGCTG
CTTTGTATTGCTATTATTATAAATAGACTTCACAGTTTTAGACTTGCTTAGGTGAAATTGCAATTCTTTTTACTT
TCAGTCTTAGATAACAAGTCTTCAATTATAGTACAATCACACATTGCTTAGGAATGCATCATTAGGCGATTTTGT
CATTATGCAAACATCATAGAGTGTACTTACACAAACCTAGATAGTATAGCCTTTATGTACCTAGGCCGTATGGTA
TAGTCTGTTGCTCCTAGGCCACAAACCTGTACAACTGTTACTGTACTGAATACTATAGACAGTTGTAACACAGTG
GTAAATATTTATCTAAATATATGCAAACAGAGAAAAGGTACAGTAAAAGTATGGTATAAAAGATAATGGTATACC
TGTGTAGGCCACTTACCACGAATGGAGCTTGCAGGACTAGAAGTTGCTCTGGGTGAGTCAGTGAGTGAGTGGTGA
ATTAATGTGAAGGCCTAGAACACTGTACACCACTGTAGACTATAAACACAGTACGCTGAAGCTACACCAAATTTA
TCTTAACAGTTTTTCTTCAATAAAAAATTATAACTTTTTAACTTTGTAAACTTTTTAATTTTTTAACTTTTAAAA
TACTTAGCTTGAAACACAAATACATTGTATAGCTATACAAAAATATTTTTTCTTTGTATCCTTATTCTAGAAGCT
TTTTTCTATTTTCTATTTTAAATTTTTTTTTTTACTTGTTAGTCGTTTTTGTTAAAAACTAAAACACACACACTT
TCACCTAGGCATAGACAGGATTAGGATCATCAGTATCACTCCCTTCCACCTCACTGCCTTCCACCTCCACATCTT
GTCCCACTGGAAGGTTTTTAGGGGCAATAACACACATGTAGCTGTCACCTATGATAACAGTGCTTTCTGTTGAAT
ACCTCCTGAAGGACTTGCCTGAGGCTGTTTTACATTTAACTTAAAAAAAAAAAAAGTAGAAGGAGTGCACTCTAA
AATAACAATAAAAGGCATAGTATAGTGAATACATAAACCAGCAATGTAGTAGTTTATTATCAAGTGTTGTACACT
GTAATAATTGTATGTGCTATACTTTAAATAACTTGCAAAATAGTACTAAGACCTTATGATGGTTACAGTGTCACT
AAGGCAATAGCATATTTTCAGGTCCATTGTAATCTAATGGGACTACCATCATATATGCAGTCTACCATTGACTGA
AACGTTACATGGCACATAACTGTATTTGCAAGAATGATTTGTTTTACATTAATATCACATAGGATGTACCTTTTT
AGAGTGGTATGTTTATGTGGATTAAGATGTACAAGTTGAGCAAGGGGACCAAGAGCCCTGGGTTCTGTCTTGGAT
GTGAGCGTTTATGTTCTTCTCCTCATGTCTGTTTTCTCATTAAATTCAAAGGCTTGAACGGGCCCTATTTAGCCC
TTCTGTTTTCTACGTGTTCTAAATAACTAAAGCTTTTAAATTCTAGCCATTTAGTGTAGAACTCTCTTTGCAGTG
ATGAAATGCTGTATTGGTTTCTTGGCTAGCATATTAAATATTTTTATCTTTGTCTTGATACTTCAATGTCGTTTT
AAACATCAGGATCGGGCTTCAGTATTCTCATAACCAGAGAGTTCACTGAGGATACAGGACTGTTTGCCCATTTTT
TGTTATGGCTCCAGACTTGTGGTATTTCCATGTCTTTTTTTTTTTTTTTTTTTTTGACCTTTTAGCGGCTTTAAA
GTATTTCTGTTGTTAGGTGTTGTATTACTTTTCTAAGATTACTTAACAAAGCACCACAAACTGAGTGGCTTTAAA
CAACAGCAATTTATTCTCTCACAATTCTAGAAGCTAGAAGTCCGAAATCAAAGTGTTGACAGGGGCATGATCTTC
AAGAGAGAAGACTCTTTCCTTGCCTCTTCCTGGCTTCTGGTGGTTACCAGCAATCCTGAGTGTTCCTTTCTTGCC
TTGTAGTTTCAACAATCCAGTATCTGCCTTTTGTCTTCACATGGCTGTCTACCATTTGTCTCTGTGTCTCCAAAT
CTCTCTCCTTATAAACACAGCAGTTATTGGATTAGGCCCCACTCTAATCCAGTATGACCCCATTTTAACATGATT
ACACTTATTTCTAGATAAGGTCACATTCACGTACACCAAGGGTTAGGAATTGAACATATCTTTTTGGGGGACACA
ATTCAACCCACAAGTGTCAGTCTCTAGCTGAGCCTTTCCCTTCCTGTTTTTCTCCTTTTTAGTTGCTATGGGTTA
GGGGCCAAATCTCCAGTCATACTAGAATTGCACATGGACTGGATATTTGGGAATACTGCGGGTCTATTCTATGAG
CTTTAGTATGTAACATTTAATATCAGTGTAAAGAAGCCCTTTTTTAAGTTATTTCTTTGAATTTCTAAATGTATG
CCCTGAATATAAGTAACAAGTTACCATGTCTTGTAAAATGATCATATCAACAAACATTTAATGTGCACCTACTGT
GCTAGTTGAATGTCTTTATCCTGATAGGAGATAACAGGATTCCACATCTTTGACTTAAGAGGACAAACCAAATAT
GTCTAAATCATTTGGGGTTTTGATGGATATCTTTAAATTGCTGAACCTAATCATTGGTTTCATATGTCATTGTTT
AGATATCTCCGGAGCATTTGGATAATGTGACAGTTGGAATGCAGTGATGTCGACTCTTTGCCCACCGCCATCTCC
AGCTGTTGCCAAGACAGAGATTGCTTTAAGTGGCAAATCACCTTTATTAGCAGCTACTTTTGCTTACTGGGACAA
TATTCTTGGTCCTAGAGTAAGGCACATTTGGGCTCCAAAGACAGAACAGGTACTTCTCAGTGATGGAGAAATAAC
TTTTCTTGCCAACCACACTCTAAATGGAGAAATCCTTCGAAATGCAGAGAGTGGTGCTATAGATGTAAAGTTTTT
TGTCTTGTCTGAAAAGGGAGTGATTATTGTTTCATTAATCTTTGATGGAAACTGGAATGGGGATCGCAGCACATA
TGGACTATCAATTATACTTCCACAGACAGAACTTAGTTTCTACCTCCCACTTCATAGAGTGTGTGTTGATAGATT
AACACATATAATCCGGAAAGGAAGAATATGGATGCATAAGGTAAGTGATTTTTCAGCTTATTAATCATGTTAACC
TATCTGTTGAAAGCTTATTTTCTGGTACATATAAATCTTATTTTTTTAATTATATGCAGTGAACATCAAACAATA
AATGTTATTTATTTTGCATTTACCCTATTAGATACAAATACATCTGGTCTGATACCTGTCATCTTCATATTAACT
GTGGAAGGTACGAAATGGTAGCTCCACATTATAGATGAAAAGCTAAAGCTTAGACAAATAAAGAAACTTTTAGAC
CCTGGATTCTTCTTGGGAGCCTTTGACTCTAATACCTTTTGTTTCCCTTTCATTGCACAATTCTGTCTTTTGCTT
ACTACTATGTGTAAGTATAACAGTTCAAAGTAATAGTTTCATAAGCTGTTGGTCATGTAGCCTTTGGTCTCTTTA
ACCTCTTTGCCAAGTTCCCAGGTTCATAAAATGAGGAGGTTGAATGGAATGGTTCCCAAGAGAATTCCTTTTAAT
CTTACAGAAATTATTGTTTTCCTAAATCCTGTAGTTGAATATATAATGCTATTTACATTTCAGTATAGTTTTGAT
GTATCTAAAGAACACATTGAATTCTCCTTCCTGTGTTCCAGTTTGATACTAACCTGAAAGTCCATTAAGCATTAC
CAGTTTTAAAAGGCTTTTGCCCAATAGTAAGGAAAAATAATATCTTTTAAAAGAATAATTTTTTACTATGTTTGC
AGGCTTACTTCCTTTTTTCTCACATTATGAAACTCTTAAAATCAGGAGAATCTTTTAAACAACATCATAATGTTT
AATTTGAAAAGTGCAAGTCATTCTTTTCCTTTTTGAAACTATGCAGATGTTACATTGACTGTTTTCTGTGAAGTT
ATCTTTTTTTCACTGCAGAATAAAGGTTGTTTTGATTTTATTTTGTATTGTTTATGAGAACATGCATTTGTTGGG
TTAATTTCCTACCCCTGCCCCCATTTTTTCCCTAAAGTAGAAAGTATTTTTCTTGTGAACTAAATTACTACACAA
GAACATGTCTATTGAAAAATAAGCAAGTATCAAAATGTTGTGGGTTGTTTTTTTAAATAAATTTTCTCTTGCTCA
GGAAAGACAAGAAAATGTCCAGAAGATTATCTTAGAAGGCACAGAGAGAATGGAAGATCAGGTATATGCAAATTG
CATACTGTCAAATGTTTTTCTCACAGCATGTATCTGTATAAGGTTGATGGCTACATTTGTCAAGGCCTTGGAGAC
ATACGAATAAGCCTTTAATGGAGCTTTTATGGAGGTGTACAGAATAAACTGGAGGAAGATTTCCATATCTTAAAC
CCAAAGAGTTAAATCAGTAAACAAAGGAAAATAGTAATTGCATCTACAAATTAATATTTGCTCCCTTTTTTTTTC
TGTTTGCCCAGAATAAATTTTGGATAACTTGTTCATAGTAAAAATAAAAAAAATTGTCTCTGATATGTTCTTTAA
GGTACTACTTCTCGAACCTTTCCCTAGAAGTAGCTGTAACAGAAGGAGAGCATATGTACCCCTGAGGTATCTGTC
TGGGGTGTAGGCCCAGGTCCACACAATATTTCTTCTAAGTCTTATGTTGTATCGTTAAGACTCATGCAATTTACA
TTTTATTCCATAACTATTTTAGTATTAAAATTTGTCAGTGATATTTCTTACCCTCTCCTCTAGGAAAATGTGCCA
TGTTTATCCCTTGGCTTTGAATGCCCCTCAGGAACAGACACTAAGAGTTTGAGAAGCATGGTTACAAGGGTGTGG
CTTCCCCTGCGGAAACTAAGTACAGACTATTTCACTGTAAAGCAGAGAAGTTCTTTTGAAGGAGAATCTCCAGTG
AAGAAAGAGTTCTTCACTTTTACTTCCATTTCCTCTTGTGGGTGACCCTCAATGCTCCTTGTAAAACTCCAATAT
TTTAAACATGGCTGTTTTGCCTTTCTTTGCTTCTTTTTAGCATGAATGAGACAGATGATACTTTAAAAAAGTAAT
TAAAAAAAAAAACTTGTGAAAATACATGGCCATAATACAGAACCCAATACAATGATCTCCTTTACCAAATTGTTA
TGTTTGTACTTTTGTAGATAGCTTTCCAATTCAGAGACAGTTATTCTGTGTAAAGGTCTGACTTAACAAGAAAAG
ATTTCCCTTTACCCAAAGAATCCCAGTCCTTATTTGCTGGTCAATAAGCAGGGTCCCCAGGAATGGGGTAACTTT
CAGCACCCTCTAACCCACTAGTTATTAGTAGACTAATTAAGTAAACTTATCGCAAGTTGAGGAAACTTAGAACCA
ACTAAAATTCTGCTTTTACTGGGATTTTGTTTTTTCAAACCAGAAACCTTTACTTAAGTTGACTACTATTAATGA
ATTTTGGTCTCTCTTTTAAGTGCTCTTCTTAAAAATGTTATCTTACTGCTGAGAAGTTCAAGTTTGGGAAGTACA
AGGAGGAATAGAAACTTAAGAGATTTTCTTTTAGAGCCTCTTCTGTATTTAGCCCTGTAGGATTTTTTTTTTTTT
TTTTTTTTTTGGTGTTGTTGAGCTTCAGTGAGGCTATTCATTCACTTATACTGATAATGTCTGAGATACTGTGAA
TGAAATACTATGTATGCTTAAACCTAAGAGGAAATATTTTCCCAAAATTATTCTTCCCGAAAAGGAGGAGTTGCC
TTTTGATTGAGTTCTTGCAAATCTCACAACGACTTTATTTTGAACAATACTGTTTGGGGATGATGCATTAGTTTG
AAACAACTTCAGTTGTAGCTGTCATCTGATAAAATTGCTTCACAGGGAAGGAAATTTAACACGGATCTAGTCATT
ATTCTTGTTAGATTGAATGTGTGAATTGTAATTGTAAACAGGCATGATAATTATTACTTTAAAAACTAAAAACAG
TGAATAGTTAGTTGTGGAGGTTACTAAAGGATGGTTTTTTTTTAAATAAAACTTTCAGCATTATGCAAATGGGCA
TATGGCTTAGGATAAAACTTCCAGAAGTAGCATCACATTTAAATTCTCAAGCAACTTAATAATATGGGGCTCTGA
AAAACTGGTTAAGGTTACTCCAAAAATGGCCCTGGGTCTGACAAAGATTCTAACTTAAAGATGCTTATGAAGACT
TTGAGTAAAATCATTTCATAAAATAAGTGAGGAAAAACAACTAGTATTAAATTCATCTTAAATAATGTATGATTT
AAAAAATATGTTTAGCTAAAAATGCATAGTCATTTGACAATTTCATTTATATCTCAAAAAATTTACTTAACCAAG
TTGGTCACAAAACTGATGAGACTGGTGGTGGTAGTGAATAAATGAGGGACCATCCATATTTGAGACACTTTACAT
TTGTGATGTGTTATACTGAATTTTCAGTTTGATTCTATAGACTACAAATTTCAAAATTACAATTTCAAGATGTAA
TAAGTAGTAATATCTTGAAATAGCTCTAAAGGGAATTTTTCTGTTTTATTGATTCTTAAAATATATGTGCTGATT
TTGATTTGCATTTGGGTAGATTATACTTTTATGAGTATGGAGGTTAGGTATTGATTCAAGTTTTCCTTACCTATT
TGGTAAGGATTTCAAAGTCTTTTTGTGCTTGGTTTTCCTCATTTTTAAATATGAAATATATTGATGACCTTTAAC
AAATTTTTTTTATCTCAAATTTTAAAGGAGATCTTTTCTAAAAGAGGCATGATGACTTAATCATTGCATGTAACA
GTAAACGATAAACCAATGATTCCATACTCTCTAAAGAATAAAAGTGAGCTTTAGGGCCGGGCATGGTCAGAAATT
TGACACCAACCTGGCCAACATGGCGAAACCCCGTCTCTACTAAAAATACAAAAATCAGCCGGGCATGGTGGCGGC
ACCTATAGTCCCAGCTACTTGGGAGGATGAGACAGGAGAGTCACTTGAACCTGGGAGGAGAGGTTGCAGTGAGCT
GAGATCACGCCATTGCACTCCAGCCTGAGCAATGAAAGCAAAACTCCATCTCAAAAAAAAAAAAAGAAAAGAAAG
AATAAAAGTGAGCTTTGGATTGCATATAAATCCTTTAGACATGTAGTAGACTTGTTTGATACTGTGTTTGAACAA
ATTACGAAGTATTTTCATCAAAGAATGTTATTGTTTGATGTTATTTTTATTTTTTATTGCCCAGCTTCTCTCATA
TTACGTGATTTTCTTCACTTCATGTCACTTTATTGTGCAGGGTCAGAGTATTATTCCAATGCTTACTGGAGAAGT
GATTCCTGTAATGGAACTGCTTTCATCTATGAAATCACACAGTGTTCCTGAAGAAATAGATGTAAGTTTAAATGA
GAGCAATTATACACTTTATGAGTTTTTTGGGGTTATAGTATTATTATGTATATTATTAATATTCTAATTTTAATA
GTAAGGACTTTGTCATACATACTATTCACATACAGTATTAGCCACTTTAGCAAATAAGCACACACAAAATCCTGG
ATTTTATGGCAAAACAGAGGCATTTTTGATCAGTGATGACAAAATTAAATTCATTTTGTTTATTTCATTACTTTT
ATAATTCCTAAAAGTGGGAGGATCCCAGCTCTTATAGGAGCAATTAATATTTAATGTAGTGTCTTTTGAAACAAA
ACTGTGTGCCAAAGTAGTAACCATTAATGGAAGTTTACTTGTAGTCACAAATTTAGTTTCCTTAATCATTTGTTG
AGGACGTTTTGAATCACACACTATGAGTGTTAAGAGATACCTTTAGGAAACTATTCTTGTTGTTTTCTGATTTTG
TCATTTAGGTTAGTCTCCTGATTCTGACAGCTCAGAAGAGGAAGTTGTTCTTGTAAAAATTGTTTAACCTGCTTG
ACCAGCTTTCACATTTGTTCTTCTGAAGTTTATGGTAGTGCACAGAGATTGTTTTTTGGGGAGTCTTGATTCTCG
GAAATGAAGGCAGTGTGTTATATTGAATCCAGACTTCCGAAAACTTGTATATTAAAAGTGTTATTTCAACACTAT
GTTACAGCCAGACTAATTTTTTTATTTTTTGATGCATTTTAGATAGCTGATACAGTACTCAATGATGATGATATT
GGTGACAGCTGTCATGAAGGCTTTCTTCTCAAGTAAGAATTTTTCTTTTCATAAAAGCTGGATGAAGCAGATACC
ATCTTATGCTCACCTATGACAAGATTTGGAAGAAAGAAAATAACAGACTGTCTACTTAGATTGTTCTAGGGACAT
TACGTATTTGAACTGTTGCTTAAATTTGTGTTATTTTTCACTCATTATATTTCTATATATATTTGGTGTTATTCC
ATTTGCTATTTAAAGAAACCGAGTTTCCATCCCAGACAAGAAATCATGGCCCCTTGCTTGATTCTGGTTTCTTGT
TTTACTTCTCATTAAAGCTAACAGAATCCTTTCATATTAAGTTGTACTGTAGATGAACTTAAGTTATTTAGGCGT
AGAACAAAATTATTCATATTTATACTGATCTTTTTCCATCCAGCAGTGGAGTTTAGTACTTAAGAGTTTGTGCCC
TTAAACCAGACTCCCTGGATTAATGCTGTGTACCCGTGGGCAAGGTGCCTGAATTCTCTATACACCTATTTCCTC
ATCTGTAAAATGGCAATAATAGTAATAGTACCTAATGTGTAGGGTTGTTATAAGCATTGAGTAAGATAAATAATA
TAAAGCACTTAGAACAGTGCCTGGAACATAAAAACACTTAATAATAGCTCATAGCTAACATTTCCTATTTACATT
TCTTCTAGAAATAGCCAGTATTTGTTGAGTGCCTACATGTTAGTTCCTTTACTAGTTGCTTTACATGTATTATCT
TATATTCTGTTTTAAAGTTTCTTCACAGTTACAGATTTTCATGAAATTTTACTTTTAATAAAAGAGAAGTAAAAG
TATAAAGTATTCACTTTTATGTTCACAGTCTTTTCCTTTAGGCTCATGATGGAGTATCAGAGGCATGAGTGTGTT
TAACCTAAGAGCCTTAATGGCTTGAATCAGAAGCACTTTAGTCCTGTATCTGTTCAGTGTCAGCCTTTCATACAT
CATTTTAAATCCCATTTGACTTTAAGTAAGTCACTTAATCTCTCTACATGTCAATTTCTTCAGCTATAAAATGAT
GGTATTTCAATAAATAAATACATTAATTAAATGATATTATACTGACTAATTGGGCTGTTTTAAGGCTCAATAAGA
AAATTTCTGTGAAAGGTCTCTAGAAAATGTAGGTTCCTATACAAATAAAAGATAACATTGTGCTTATAGCTTCGG
TGTTTATCATATAAAGCTATTCTGAGTTATTTGAAGAGCTCACCTACTTTTTTTTGTTTTTAGTTTGTTAAATTG
TTTTATAGGCAATGTTTTTAATCTGTTTTCTTTAACTTACAGTGCCATCAGCTCACACTTGCAAACCTGTGGCTG
TTCCGTTGTAGTAGGTAGCAGTGCAGAGAAAGTAAATAAGGTAGTTTATTTTATAATCTAGCAAATGATTTGACT
CTTTAAGACTGATGATATATCATGGATTGTCATTTAAATGGTAGGTTGCAATTAAAATGATCTAGTAGTATAAGG
AGGCAATGTAATCTCATCAAATTGCTAAGACACCTTGTGGCAACAGTGAGTTTGAAATAAACTGAGTAAGAATCA
TTTATCAGTTTATTTTGATAGCTCGGAAATACCAGTGTCAGTAGTGTATAAATGGTTTTGAGAATATATTAAAAT
CAGATATATAAAAAAAATTACTCTTCTATTTCCCAATGTTATCTTTAACAAATCTGAAGATAGTCATGTACTTTT
GGTAGTAGTTCCAAAGAAATGTTATTTGTTTATTCATCTTGATTTCATTGTCTTCGCTTTCCTTCTAAATCTGTC
CCTTCTAGGGAGCTATTGGGATTAAGTGGTCATTGATTATTATACTTTATTCAGTAATGTTTCTGACCCTTTCCT
TCAGTGCTACTTGAGTTAATTAAGGATTAATGAACAGTTACATTTCCAAGCATTAGCTAATAAACTAAAGGATTT
TGCACTTTTCTTCACTGACCATTAGTTAGAAAGAGTTCAGAGATAAGTATGTGTATCTTTCAATTTCAGCAAACC
TAATTTTTTAAAAAAAGTTTTACATAGGAAATATGTTGGAAATGATACTTTACAAAGATATTCATAATTTTTTTT
TGTAATCAGCTACTTTGTATATTTACATGAGCCTTAATTTATATTTCTCATATAACCATTTATGAGAGCTTAGTA
TACCTGTGTCATTATATTGCATCTACGAACTAGTGACCTTATTCCTTCTGTTACCTCAAACAGGTGGCTTTCCAT
CTGTGATCTCCAAAGCCTTAGGTTGCACAGAGTGACTGCCGAGCTGCTTTATGAAGGGAGAAAGGCTCCATAGTT
GGAGTGTTTTTTTTTTTTTTTTTAAACATTTTTCCCATCCTCCATCCTCTTGAGGGAGAATAGCTTACCTTTTAT
CTTGTTTTAATTTGAGAAAGAAGTTGCCACCACTCTAGGTTGAAAACCACTCCTTTAACATAATAACTGTGGATA
TGGTTTGAATTTCAAGATAGTTACATGCCTTTTTATTTTTCCTAATAGAGCTGTAGGTCAAATATTATTAGAATC
AGATTTCTAAATCCCACCCAATGACCTGCTTATTTTAAATCAAATTCAATAATTAATTCTCTTCTTTTTGGAGGA
TCTGGACATTCTTTGATATTTCTTACAACGAATTTCATGTGTAGACCCACTAAACAGAAGCTATAAAAGTTGCAT
GGTCAAATAAGTCTGAGAAAGTCTGCAGATGATATAATTCACCTGAAGAGTCACAGTATGTAGCCAAATGTTAAA
GGTTTTGAGATGCCATACAGTAAATTTACCAAGCATTTTCTAAATTTATTTGACCACAGAATCCCTATTTTAAGC
AACAACTGTTACATCCCATGGATTCCAGGTGACTAAAGAATACTTATTTCTTAGGATATGTTTTATTGATAATAA
CAATTAAAATTTCAGATATCTTTCATAAGCAAATCAGTGGTCTTTTTACTTCATGTTTTAATGCTAAAATATTTT
CTTTTATAGATAGTCAGAACATTATGCCTTTTTCTGACTCCAGCAGAGAGAAAATGCTCCAGGTTATGTGAAGCA
GAATCATCATTTAAATATGAGTCAGGGCTCTTTGTACAAGGCCTGCTAAAGGTATAGTTTCTAGTTATCACAAGT
GAAACCACTTTTCTAAAATCATTTTTGAGACTCTTTATAGACAAATCTTAAATATTAGCATTTAATGTATCTCAT
ATTGACATGCCCAGAGACTGACTTCCTTTACACAGTTCTGCACATAGACTATATGTCTTATGGATTTATAGTTAG
TATCATCAGTGAAACACCATAGAATACCCTTTGTGTTCCAGGTGGGTCCCTGTTCCTACATGTCTAGCCTCAGGA
CTTTTTTTTTTTTAACACATGCTTAAATCAGGTTGCACATCAAAAATAAGATCATTTCTTTTTAACTAAATAGAT
TTGAATTTTATTGAAAAAAAATTTTAAACATCTTTAAGAAGCTTATAGGATTTAAGCAATTCCTATGTATGTGTA
CTAAAATATATATATTTCTATATATAATATATATTAGAAAAAAATTGTATTTTTCTTTTATTTGAGTCTACTGTC
AAGGAGCAAAACAGAGAAATGTAAATTAGCAATTATTTATAATACTTAAAGGGAAGAAAGTTGTTCACCTTGTTG
AATCTATTATTGTTATTTCAATTATAGTCCCAAGACGTGAAGAAATAGCTTTCCTAATGGTTATGTGATTGTCTC
ATAGTGACTACTTTCTTGAGGATGTAGCCACGGCAAAATGAAATAAAAAAATTTAAAAATTGTTGCAAATACAAG
TTATATTAGGCTTTTGTGCATTTTCAATAATGTGCTGCTATGAACTCAGAATGATAGTATTTAAATATAGAAACT
AGTTAAAGGAAACGTAGTTTCTATTTGAGTTATACATATCTGTAAATTAGAACTTCTCCTGTTAAAGGCATAATA
AAGTGCTTAATACTTTTGTTTCCTCAGCACCCTCTCATTTAATTATATAATTTTAGTTCTGAAAGGGACCTATAC
CAGATGCCTAGAGGAAATTTCAAAACTATGATCTAATGAAAAAATATTTAATAGTTCTCCATGCAAATACAAATC
ATATAGTTTTCCAGAAAATACCTTTGACATTATACAAAGATGATTATCACAGCATTATAATAGTAAAAAAATGGA
AATAGCCTCTTTCTTCTGTTCTGTTCATAGCACAGTGCCTCATACGCAGTAGGTTATTATTACATGGTAACTGGC
TACCCCAACTGATTAGGAAAGAAGTAAATTTGTTTTATAAAAATACATACTCATTGAGGTGCATAGAATAATTAA
GAAATTAAAAGACACTTGTAATTTTGAATCCAGTGAATACCCACTGTTAATATTTGGTATATCTCTTTCTAGTCT
TTTTTTCCCTTTTGCATGTATTTTCTTTAAGACTCCCACCCCCACTGGATCATCTCTGCATGTTCTAATCTGCTT
TTTTCACAGCAGATTCTAAGCCTCTTTGAATATCAACACAAACTTCAACAACTTCATCTATAGATGCCAAATAAT
AAATTCATTTTTATTTACTTAACCACTTCCTTTGGATGCTTAGGTCATTCTGATGTTTTGCTATTGAAACCAATG
CTATACTGAACACTTCTGTCACTAAAACTTTGCACACACTCATGAATAGCTTCTTAGGATAAATTTTTAGAGATG
GATTTGCTAAATCAGAGACCATTTTTTAAAATTAAAAAACAATTATTCATATCGTTTGGCATGTAAGACAGTAAA
TTTTCCTTTTATTTTGACAGGATTCAACTGGAAGCTTTGTGCTGCCTTTCCGGCAAGTCATGTATGCTCCATATC
CCACCACACACATAGATGTGGATGTCAATACTGTGAAGCAGATGCCACCCTGTCATGAACATATTTATAATCAGC
GTAGATACATGAGATCCGAGCTGACAGCCTTCTGGAGAGCCACTTCAGAAGAAGACATGGCTCAGGATACGATCA
TCTACACTGACGAAAGCTTTACTCCTGATTTGTACGTAATGCTCTGCCTGCTGGTACTGTAGTCAAGCAATATGA
AATTGTGTCTTTTACGAATAAAAACAAAACAGAAGTTGCATTTAAAAAGAAAGAAATATTACCAGCAGAATTATG
CTTGAAGAAACATTTAATCAAGCATTTTTTTCTTAAATGTTCTTCTTTTTCCATACAATTGTGTTTACCCTAAAA
TAGGTAAGATTAACCCTTAAAGTAAATATTTAACTATTTGTTTAATAAATATATATTGAGCTCCTAGGCACTGTT
CTAGGTACCGGGCTTAATAGTGGCCAACCAGACAGCCCCAGCCCCAGCCCCTACATTGTGTATAGTCTATTATGT
AACAGTTATTGAATGGACTTATTAACAAAACCAAAGAAGTAATTCTAAGTCTTTTTTTTCTTGACATATGAATAT
AAAATACAGCAAAACTGTTAAAATATATTAATGGAACATTTTTTTACTTTGCATTTTATATTGTTATTCACTTCT
TATTTTTTTTTAAAAAAAAAAGCCTGAACAGTAAATTCAAAAGGAAAAGTAATGATAATTAATTGTTGAGCATGG
ACCCAACTTGAAAAAAAAAATGATGATGATAAATCTATAATCCTAAAACCCTAAGTAAACACTTAAAAGATGTTC
TGAAATCAGGAAAAGAATTATAGTATACTTTTGTGTTTCTCTTTTATCAGTTGAAAAAAGGCACAGTAGCTCATG
CCTGTAAGAACAGAGCTTTGGGAGTGCAAGGCAGGCGGATCACTTGAGGCCAGGAGTTCCAGACCAGCCTGGGCA
ACATAGTGAAACCCCATCTCTACAAAAAATAAAAAAGAATTATTGGAATGTGTTTCTGTGTGCCTGTAATCCTAG
CTATTCCGAAAGCTGAGGCAGGAGGATCTTTTGAGCCCAGGAGTTTGAGGTTACAGGGAGTTATGATGTGCCAGT
GTACTCCAGCCTGGGGAACACCGAGACTCTGTCTTATTTAAAAAAAAAAAAAAAAAAATGCTTGCAATAATGCCT
GGCACATAGAAGGTAACAGTAAGTGTTAACTGTAATAACCCAGGTCTAAGTGTGTAAGGCAATAGAAAAATTGGG
GCAAATAAGCCTGACCTATGTATCTACAGAATCAGTTTGAGCTTAGGTAACAGACCTGTGGAGCACCAGTAATTA
CACAGTAAGTGTTAACCAAAAGCATAGAATAGGAATATCTTGTTCAAGGGACCCCCAGCCTTATACATCTCAAGG
TGCAGAAAGATGACTTAATATAGGACCCATTTTTTCCTAGTTCTCCAGAGTTTTTATTGGTTCTTGAGAAAGTAG
TAGGGGAATGTTTTAGAAAATGAATTGGTCCAACTGAAATTACATGTCAGTAAGTTTTTATATATTGGTAAATTT
TAGTAGACATGTAGAAGTTTTCTAATTAATCTGTGCCTTGAAACATTTTCTTTTTTCCTAAAGTGCTTAGTATTT
TTTCCGTTTTTTGATTGGTTACTTGGGAGCTTTTTTGAGGAAATTTAGTGAACTGCAGAATGGGTTTGCAACCAT
TTGGTATTTTTGTTTTGTTTTTTAGAGGATGTATGTGTATTTTAACATTTCTTAATCATTTTTAGCCAGCTATGT
TTGTTTTGCTGATTTGACAAACTACAGTTAGACAGCTATTCTCATTTTGCTGATCATGACAAAATAATATCCTGA
ATTTTTAAATTTTGCATCCAGCTCTAAATTTTCTAAACATAAAATTGTCCAAAAAATAGTATTTTCAGCCACTAG
ATTGTGTGTTAAGTCTATTGTCACAGAGTCATTTTACTTTTAAGTATATGTTTTTACATGTTAATTATGTTTGTT
ATTTTTAATTTTAACTTTTTAAAATAATTCCAGTCACTGCCAATACATGAAAAATTGGTCACTGGAATTTTTTTT
TTGACTTTTATTTTAGGTTCATGTGTACATGTGCAGGTGTGTTATACAGGTAAATTGCGTGTCATGAGGGTTTGG
TGTACAGGTGATTTCATTACCCAGGTAATAAGCATAGTACCCAATAGGTAGTTTTTTGATCCTCACCCTTCTCCC
ACCCTCAAGTAGGCCCTGGTGTTGCTGTTTCCTTCTTTGTGTCCATGTATACTCAGTGTTTAGCTCCCACTTAGA
AGTGAGAACATGCGGTAGTTGGTTTTCTGTTCCTGGATTAGTTCACTTAGGATAATGACCTCTAGCTCCATCTGG
TTTTTATGGCTGCATAGTATTCCATGGTGTATATGTATCACATTTTCTTTATCCAGTCTACCATTGATAGGCATT
TAGGTTGATTCCCTGTCTTTGTTATCATGAATAGTGCTGTGATGAACATACACATGCATGTGTCTTTATGGTAGA
AAAATTTGTATTCCTTTAGGTACATATAGAATAATGGGGTTGCTAGGGTGAATGGTAGTTCTATTTTCAGTTATT
TGAGAAATCTTCAAACTGCTTTTCATAATAGCTAAACTAATTTACAGTCCCGCCAGCAGTGTATAAGTGTTCCCT
TTTCTCCACAACCTTGCCAACATCTGTGATTTTTTGACTTTTTAATAATAGCCATTCCTAGAGAATTGATTTGCA
ATTCTCTATTAGTGATATTAAGCATTTTTTCATATGCTTTTTAGCTGTCTGTATATATTCTTCTGAAAAATTTTC
ATGTCCTTTGCCCAGTTTGTAGTGGGGTGGGTTGTTTTTTGCTTGTTAATTAGTTTTAAGTTCCTTCCAGATTCT
GCATATCCCTTTGTTGGATACATGGTTTGCAGATATTTTTCTCCCATTGTGTAGGTTGTCTTTTACTCTGTTGAT
AGTTTCTTTTGCCATGCAGGAGCTCGTTAGGTCCCATTTGTGTTTGTTTTTGTTGCAGTTGCTTTTGGCGTCTTC
ATCATAAAATCTGTGCCAGGGCCTATGTCCAGAATGGTATTTCCTAGGTTGTCTTCCAGGGTTTTTACAATTTTA
GATTTTACGTTTATGTCTTTAATCCATCTTGAGTTGATTTTTGTATATGGCACAAGGAAGGGGTCCAGTTTCACT
CCAATTCCTATGGCTAGCAATTATCCCAGCACCATTTATTGAATACGGAGTCCTTTCCCCATTGCTTGTTTTTTG
TCAACTTTGTTGAAGATCAGATGGTTGTAAGTGTGTGGCTTTATTTCTTGGCTCTCTATTCTCCATTGGTCTATG
TGTCTGTTTTTATAACAGTACCCTGCTGTTCAGGTTCCTATAGCCTTTTAGTATAAAATCGGCTAATGTGATGCC
TCCAGCTTTGTTCTTTTTGCTTAGGATTGCTTTGGCTATTTGGGCTCCTTTTTGGGTCCATATTAATTTTAAAAC
AGTTTTTTCTGGTTTTGTGAAGGATATCATTGGTAGTTTATAGGAATAGCATTGAATCTGTAGATTGCTTTGGGC
AGTATGGCCATTTTAACAATATTAATTCTTCCTATCTATGAATATGGAATGTTTTTCCATGTGTTTGTGTCATCT
CTTTATACCTGATGTATAAAGAAAAGCTGGTATTATTCCTACTCAATCTGTTCCAAAAAATTGAGGAGGAGGAAC
TCTTCCCTAATGAGGCCAGCATCATTCTGATACCAAAACCTGGCAGAGACACAACAGAAAAAAGAAAACTTCAGG
CCAATATCCTTGATGAATATAGATGCAAAAATCCTCAACAAAATACTAGCAAACCAAATCCAGCAGCACATCAAA
AAGCTGATCTACTTTGATCAAGTAGGCTTTATCCCTGGGATGCAAGGTTGGTTCAACATACACAAATCAATAAGT
GTGATTCATCACATAAACAGAGCTAAAAACAAAAACCACAAGATTATCTCAATAGGTAGAGAAAAGGTTGTCAAT
AAAATTTAACATCCTCCATGTTAAAAACCTTCAGTAGGTCAGGTGTAGTGACTCACACCTGTAATCCCAGCACTT
TGGGAGGCCAAGGCGGGCATATCTCTTAAGCCCAGGAGTTCAAGACGAGCCTAGGCAGCATGGTGAAACCCCATC
TCTACAAAAAAAAAAAAAAAAAAAAATTAGCTTGGTATGGTGACATGCACCTATAGTCCCAGCTATTCAGGAGGT
TGAGGTGGGAGGATTGTTTGAGCCCGGGAGGCAGAGGTTGGCAGCGAGCTGAGATCATGCCACCGCACTCCAGCC
TGGGCAACGGAGTGAGACCCTGTCTCAAAAAAGAAAAATCACAAACAATCCTAAACAAACTAGGCATTGAAGGAA
CATGCCTCAAAAAAATAAGAACCATCTATGACAGACCCATAGCCAATATCTTACCAAATGGGCAAAAGCTGGAAG
TATTCTCCTTGAGAACCGTAACAAGACAAGGATGTCCACTCTCACCACTCCTTTTCAGCATAGTTCTGGAAGTCC
TAGCCAGAGCAATCAGGAAAGAGAAAGAAAGAAAGACATTCAGATAGGAAGAGAAGAAGTCAAACTATTTCTGTT
TGCAGGCAGTATAATTCTGTACCTAGAAAATCTCATAGTCTCTGCCCAGAAACTCCTAAATCTGTTAAAAATTTC
AGCAAAGTTTTGGCATTCTCTATACTCCAACACCTTCCAAAGTGAGAGCAAAATCAAGAACACAGTCCCATTCAC
AATAGCCGCAAAACGAATAAAATACCTAGGAATCCAGCTAACCAGGGAGGTGAAAGATCTCTATGAGAATTACAA
AACACTGCTGAAAGAAATCAGAGATGACACAAACAAATGGAAATGTTCTTTTTTAACACCTTGCTTTATCTAATT
CACTTATGATGAAGATACTCATTCAGTGGAACAGGTATAATAAGTCCACTCGATTAAATATAAGCCTTATTCTCT
TTCCAGAGCCCAAGAAGGGGCACTATCAGTGCCCAGTCAATAATGACGAAATGCTAATATTTTTCCCCTTTACGG
TTTCTTTCTTCTGTAGTGTGGTACACTCGTTTCTTAAGATAAGGAAACTTGAACTACCTTCCTGTTTGCTTCTAC
ACATACCCATTCTCTTTTTTTGCCACTCTGGTCAGGTATAGGATGATCCCTACCACTTTCAGTTAAAAACTCCTC
CTCTTACTAAATGTTCTCTTACCCTCTGGCCTGAGTAGAACCTAGGGAAAATGGAAGAGAAAAAGATGAAAGGGA
GGTGGGGCCTGGGAAGGGAATAAGTAGTCCTGTTTGTTTGTGTGTTTGCTTTAGCACCTGCTATATCCTAGGTGC
TGTGTTAGGCACACATTATTTTAAGTGGCCATTATATTACTACTACTCACTCTGGTCGTTGCCAAGGTAGGTAGT
ACTTTCTTGGATAGTTGGTTCATGTTACTTACAGATGGTGGGCTTGTTGAGGCAAACCCAGTGGATAATCATCGG
AGTGTGTTCTCTAATCTCACTCAAATTTTTCTTCACATTTTTTGGTTTGTTTTGGTTTTTGATGGTAGTGGCTTA
TTTTTGTTGCTGGTTTGTTTTTTGTTTTTTTTTGAGATGGCAAGAATTGGTAGTTTTATTTATTAATTGCCTAAG
GGTCTCTACTTTTTTTAAAAGATGAGAGTAGTAAAATAGATTGATAGATACATACATACCCTTACTGGGGACTGC
TTATATTCTTTAGAGAAAAAATTACATATTAGCCTGACAAACACCAGTAAAATGTAAATATATCCTTGAGTAAAT
AAATGAATGTATATTTTGTGTCTCCAAATATATATATCTATATTCTTACAAATGTGTTTATATGTAATATCAATT
TATAAGAACTTAAAATGTTGGCTCAAGTGAGGGATTGTGGAAGGTAGCATTATATGGCCATTTCAACATTTGAAC
TTTTTTCTTTTCTTCATTTTCTTCTTTTCTTCAGGAATATTTTTCAAGATGTCTTACACAGAGACACTCTAGTGA
AAGCCTTCCTGGATCAGGTAAATGTTGAACTTGAGATTGTCAGAGTGAATGATATGACATGTTTTCTTTTTTAAT
ATATCCTACAATGCCTGTTCTATATATTTATATTCCCCTGGATCATGCCCCAGAGTTCTGCTCAGCAATTGCAGT
TAAGTTAGTTACACTACAGTTCTCAGAAGAGTCTGTGAGGGCATGTCAAGTGCATCATTACATTGGTTGCCTCTT
GTCCTAGATTTATGCTTCGGGAATTCAGACCTTTGTTTACAATATAATAAATATTATTGCTATCTTTTAAAGATA
TAATAATAAGATATAAAGTTGACCACAACTACTGTTTTTTGAAACATAGAATTCCTGGTTTACATGTATCAAAGT
GAAATCTGACTTAGCTTTTACAGATATAATATATACATATATATATCCTGCAATGCTTGTACTATATATGTAGTA
CAAGTATATATATATGTTTGTGTGTGTATATATATATAGTACGAGCATATATACATATTACCAGCATTGTAGGAT
ATATATATGTTTATATATTAAAAAAAAGTTATAAACTTAAAACCCTATTATGTTATGTAGAGTATATGTTATATA
TGATATGTAAAATATATAACATATACTCTATGATAGAGTGTAATATATTTTTTATATATATTTTAACATTTATAA
AATGATAGAATTAAGAATTGAGTCCTAATCTGTTTTATTAGGTGCTTTTTGTAGTGTCTGGTCTTTCTAAAGTGT
CTAAATGATTTTTCCTTTTGACTTATTAATGGGGAAGAGCCTGTATATTAACAATTAAGAGTGCAGCATTCCATA
CGTCAAACAACAAACATTTTAATTCAAGCATTAACCTATAACAAGTAAGTTTTTTTTTTTTTTTTGAGAAAGGGA
GGTTGTTTATTTGCCTGAAATGACTCAAAAATATTTTTGAAACATAGTGTACTTATTTAAATAACATCTTTATTG
TTTCATTCTTTTAAAAAATATCTACTTAATTACACAGTTGAAGGAAATCGTAGATTATATGGAACTTATTTCTTA
ATATATTACAGTTTGTTATAATAACATTCTGGGGATCAGGCCAGGAAACTGTGTCATAGATAAAGCTTTGAAATA
ATGAGATCCTTATGTTTACTAGAAATTTTGGATTGAGATCTATGAGGTCTGTGACATATTGCGAAGTTCAAGGAA
AATTCGTAGGCCTGGAATTTCATGCTTCTCAAGCTGACATAAAATCCCTCCCACTCTCCACCTCATCATATGCAC
ACATTCTACTCCTACCCACCCACTCCACCCCCTGCAAAAGTACAGGTATATGAATGTCTCAAAACCATAGGCTCA
TCTTCTAGGAGCTTCAATGTTATTTGAAGATTTGGGCAGAAAAAATTAAGTAATACGAAATAACTTATGTATGAG
TTTTAAAAGTGAAGTAAACATGGATGTATTCTGAAGTAGAATGCAAAATTTGAATGCATTTTTAAAGATAAATTA
GAAAACTTCTAAAAACTGTCAGATTGTCTGGGCCTGGTGGCTTATGCCTGTAATCCCAGCACTTTGGGAGTCCGA
GGTGGGTGGATCACAAGGTCAGGAGATCGAGACCATCCTGCCAACATGGTGAAACCCCGTCTCTACTAAGTATAC
AAAAATTAGCTGGGCGTGGCAGCGTGTGCCTGTAATCCCAGCTACCTGGGAGGCTGAGGCAGGAGAATCGCTTGA
ACCCAGGAGGTGTAGGTTGCAGTGAGTCAAGATCGCGCCACTGCACTTTAGCCTGGTGACAGAGCTAGACTCCGT
CTCAAAAAAAAAAAAAAATATCAGATTGTTCCTACACCTAGTGCTTCTATACCACACTCCTGTTAGGGGGCATCA
GTGGAAATGGTTAAGGAGATGTTTAGTGTGTATTGTCTGCCAAGCACTGTCAACACTGTCATAGAAACTTCTGTA
CGAGTAGAATGTGAGCAAATTATGTGTTGAAATGGTTCCTCTCCCTGCAGGTCTTTCAGCTGAAACCTGGCTTAT
CTCTCAGAAGTACTTTCCTTGCACAGTTTCTACTTGTCCTTCACAGAAAAGCCTTGACACTAATAAAATATATAG
AAGACGATACGTGAGTAAAACTCCTACACGGAAGAAAAACCTTTGTACATTGTTTTTTTGTTTTGTTTCCTTTGT
ACATTTTCTATATCATAATTTTTGCGCTTCTTTTTTTTTTTTTTTTTTTTTTTTTTCCATTATTTTTAGGCAGAA
GGGAAAAAAGCCCTTTAAATCTCTTCGGAACCTGAAGATAGACCTTGATTTAACAGCAGAGGGCGATCTTAACAT
AATAATGGCTCTGGCTGAGAAAATTAAACCAGGCCTACACTCTTTTATCTTTGGAAGACCTTTCTACACTAGTGT
GCAAGAACGAGATGTTCTAATGACTTTTTAAATGTGTAACTTAATAAGCCTATTCCATCACAATCATGATCGCTG
GTAAAGTAGCTCAGTGGTGTGGGGAAACGTTCCCCTGGATCATACTCCAGAATTCTGCTCTCAGCAATTGCAGTT
AAGTAAGTTACACTACAGTTCTCACAAGAGCCTGTGAGGGGATGTCAGGTGCATCATTACATTGGGTGTCTCTTT
TCCTAGATTTATGCTTTTGGGATACAGACCTATGTTTACAATATAATAAATATTATTGCTATCTTTTAAAGATAT
AATAATAGGATGTAAACTTGACCACAACTACTGTTTTTTTGAAATACATGATTCATGGTTTACATGTGTCAAGGT
GAAATCTGAGTTGGCTTTTACAGATAGTTGACTTTCTATCTTTTGGCATTCTTTGGTGTGTAGAATTACTGTAAT
ACTTCTGCAATCAACTGAAAACTAGAGCCTTTAAATGATTTCAATTCCACAGAAAGAAAGTGAGCTTGAACATAG
GATGAGCTTTAGAAAGAAAATTGATCAAGCAGATGTTTAATTGGAATTGATTATTAGATCCTACTTTGTGGATTT
AGTCCCTGGGATTCAGTCTGTAGAAATGTCTAATAGTTCTCTATAGTCCTTGTTCCTGGTGAACCACAGTTAGGG
TGTTTTGTTTATTTTATTGTTCTTGCTATTGTTGATATTCTATGTAGTTGAGCTCTGTAAAAGGAAATTGTATTT
TATGTTTTAGTAATTGTTGCCAACTTTTTAAATTAATTTTCATTATTTTTGAGCCAAATTGAAATGTGCACCTCC
TGTGCCTTTTTTCTCCTTAGAAAATCTAATTACTTGGAACAAGTTCAGATTTCACTGGTCAGTCATTTTCATCTT
GTTTTCTTCTTGCTAAGTCTTACCATGTACCTGCTTTGGCAATCATTGCAACTCTGAGATTATAAAATGCCTTAG
AGAATATACTAACTAATAAGATCTTTTTTTCAGAAACAGAAAATAGTTCCTTGAGTACTTCCTTCTTGCATTTCT
GCCTATGTTTTTGAAGTTGTTGCTGTTTGCCTGCAATAGGCTATAAGGAATAGCAGGAGAAATTTTACTGAAGTG
CTGTTTTCCTAGGTGCTACTTTGGCAGAGCTAAGTTATCTTTTGTTTTCTTAATGCGTTTGGACCATTTTGCTGG
CTATAAAATAACTGATTAATATAATTCTAACACAATGTTGACATTGTAGTTACACAAACACAAATAAATATTTTA
TTTAAAATTCTGGAAGTAATATAAAAGGGAAAATATATTTATAAGAAAGGGATAAAGGTAATAGAGCCCTTCTGC
CCCCCACCCACCAAATTTACACAACAAAATGACATGTTCGAATGTGAAAGGTCATAATAGCTTTCCCATCATGAA
TCAGAAAGATGTGGACAGCTTGATGTTTTAGACAACCACTGAACTAGATGACTGTTGTACTGTAGCTCAGTCATT
TAAAAAATATATAAATACTACCTTGTAGTGTCCCATACTGTGTTTTTTACATGGTAGATTCTTATTTAAGTGCTA
ACTGGTTATTTTCTTTGGCTGGTTTATTGTACTGTTATACAGAATGTAAGTTGTACAGTGAAATAAGTTATTAAA
GCATGTGTAAACATTGTTATATATCTTTTCTCCTAAATGGAGAATTTTGAATAAAATATATTTGAAATTTTGCCT
CTTTCAGTTGTTCATTCAGAAAAAAATACTATGATATTTGAAGACTGATCAGCTTCTGTTCAGCTGACAGTCATG
CTGGATCTAAACTTTTTTTAAAATTAATTTTGTCTTTTCAAAGAAAAAATATTTAAAGAAGCTTTATAATATAAT
CTTATGTTAAAAAAACTTTCTGCTTAACTCTCTGGATTTCATTTTGATTTTTCAAATTATATATTAATATTTCAA
ATGTAAAATACTATTTAGATAAATTGTTTTTAAACATTCTTATTATTATAATATTAATATAACCTAAACTGAAGT
TATTCATCCCAGGTATCTAATACATGTATCCAAAGTAAAAATCCAAGGAATCTGAACACTTTCATCTGCAAAGCT
AGGAATAGGTTTGACATTTTCACTCCAAGAAAAAGTTTTTTTTTGAAAATAGAATAGTTGGGATGAGAGGTTTCT
TTAAAAGAAGACTAACTGATCACATTACTATGATTCTCAAAGAAGAAACCAAAACTTCATATAATACTATAAAGT
AAATATAAAATAGTTCCTTCTATAGTATATTTCTATAATGCTACAGTTTAAACAGATCACTCTTATATAATACTA
TTTTGATTTTGATGTAGAATTGCACAAATTGATATTTCTCCTATGATCTGCAGGGTATAGCTTAAAGTAACAAAA
ACAGTCAACCACCTCCATTTAACACACAGTAACACTATGGGACTAGTTTTATTACTTCCATTTTACAAATGAGGA
AACTAAAGCTTAAAGATGTGTAATACACCGCCCAAGGTCACACAGCTGGTAAAGGTGGATTTCATCCCAGACAGT
TACAGTCATTGCCATGGGCACAGCTCCTAACTTAGTAACTCCATGTAACTGGTACTCAGTGTAGCTGAATTGAAA
GGAGAGTAAGGAAGCAGGTTTTACAGGTCTACTTGCACTATTCAGAGCCCGAGTGTGAATCCCTGCTGTGCTGCT
TGGAGAAGTTACTTAACCTATGCAAGGTTCATTTTGTAAATATTGGAAATGGAGTGATAATACGTACTTCACCAG
AGGATTTAATGAGACCTTATACGATCCTTAGTTCAGTACCTGACTAGTGCTTCATAAATGCTTTTTCATCCAATC
TGACAATCTCCAGCTTGTAATTGGGGCATTTAGAACATTTAATATGATTATTGGCATGGTAGGTTAAAGCTGTCA
TCTTGCTGTTTTCTATTTGTTCTTTTTGTTTTCTCCTTACTTTTGGATTTTTTTATTCTACTATGTCTTTTCTAT
TGTCTTATTAACTATACTCTTTGATTTATTTTAGTGGTTGTTTTAGGGTTATACCTCTTTCTAATTTACCAGTTT
ATAACCAGTTTATATACTACTTGACATATAGCTTAAGAAACTTACTGTTGTTGTCTTTTTGCTGTTATGGTCTTA
ACGTTTTTATTTCTACAAACATTATAAACTCCACACTTTATTGTTTTTTAATTTTACTTATACAGTCAATTATCT
TTTAAAGATATTTAAATATAAACATTCAAAACACCCCAATTAAAAGTCAGAGATTGTTAATACCACATGATCTCA
CTTACACACAGAATTGAAAAACTTGGAACTCATAGAAGCAGAGAGTAAAAACATGGTTACCAGGTGCTGGGGAGA
GGCGGTGGGCTGGGGAGATGTTGGTCAAAGTTAGACAGGAGGAATAAGTTCAAGAGATCTATTGTACAACTTATT
CAGTTAGATAGGAGGAATAAGCTAAAGATCAAGAGATCTATTGTACAATGTGACTATAACCAACAACATATATTG
TACACTTGAAAATTGCTAACAGTATCTTTTAAGTGTTCTCTCTACAAATAAATATGTGAGGTAATGTATATATTA
ATTAACTGTAGTCATTTCACAATGTATACTTATTTCAAAACATCATATTGTATGCTATAAATATATACAACTTTT
ATTTTTCAATTTTAGAAATGTCCTTAAAAAATCAGATTTTCAGATCAGATAAAAAAGCAAGACCCAACTATATGC
TGCCAACAGGAAACACACCTTAAAAATAAAGGACGAACAAACAGATTAAAAGTAAAAGGATGGAGAAAAGATACA
TCATATTGGTAATTAGAAGAAAACTGGAGTGACAATATGAAACAAAATAGATTTCAGAGCAAAGAATATTACCAG
GGGTAAAAATGATCATTTTATAATGATAAAAGAGTCAGTTCAGCAAAAGGATATAACAGTCCTAAATGTTTTTTC
ACCTCATAGCTGTGTCAAAATAGATGAAGCAAAAACTGATAGAACTGTAAGAAGTAGACAAGTCCACAATTATGT
TTGGAGATTTTTTTTTTTTTTTTTTTTGTCGCCCAGGCTGGAGTGCAGTGGCAGGATCTCAGCTCACTGCAAGCT
CCGCCTCCCAGGTTCACGCCATTCTCCTGCTTCAGCCTCCCCAGTAGCTGGGACTACAGGCGGCCACCACCACGC
CTGGCTAATTTTTTTGTATTTTTAGTAGAGACGGGGTTTCACCGTGTTAGCCAGGATGGTCTCGATCTCCTGACC
TCGTGATCTGCCTGCCTCGGCCTCCCAAAGTGCTGGGATTACAGGCATGAGCCACTGCACGCAGCCTGGAGATTT
TAATATCCTTTCAATGTTTAGTAGAACAAGAATACACAAAATCAGTAAGGATATAGAAGATTAGAACAAGACTAT
CAAACAATTTGACTTAAATGACATTTGTAGAGCACAGCAGTCCCCAACAACAATAAATCACACATTCTTTCCAAG
AGTACATGAAACATGTACCAAGATAGACCGTATTTTGAGCCATGAAACAAATCTTGATAAATTTAAAAGGATTCA
AGTCATAGAAAATATGTTCTCTGACCACAATGGAATTAAATTATTAACCAATAACAAATATCTGGGAAAACCTCA
AAAACTTGGACACCAGCGCTTTTAAAAGACTAAATAATTTCTAAATTATCTGTGTTGGGGGGAAAAGAGAAATGG
ATTAGAGAGCAAAAAGGGTATCAGAGTGCTGTGGTACGATTTTTATGAAGAGTGGAACAGAATCTGCCTTTGGCG
TTTCCCCACTACAGCCCATTCTTCACATTGATAACAGCATGATCCTTCTAAAATTAAATCTAACGATCACTTCTG
CTTAATGGCTCTCCAACACTTACAGAATTAGGTCCAAAATTCTAGCACAGTTTCTGTTCATCTTTCTAACCTTTC
TTCCCACAGGTCTAGCTAGTACGTATTTCTTTTATTGCATTTATTACACTATTCCTTTGCTTATCTATCTCCCCA
CCTAGGCTAAAGAACAAGATTCTTGTCTTTTTCATTTTTGTGTCTCAGTGCCTAGCATGGTGCCAGGCACACAGC
ATGCTTCCAGTAAATGTTAGCTGGATGGATGTAATGAGTATATTAAATATTAATTTATTTGTTTTTCCCCAAAAA
GAATTATTTCCTGCAAATCAAGGAAATTGCTTTCTTTATATAATCAAAAACTTATTTTCCCAGAAGATTCTTCAT
TAAAAATTAAGCCTATGCACAACCTAGCTCTAAAGTTTCAAAGATTTTAGGCAGCAATTTTTCAATCTTTTTGAA
GTAATACATTTGAATCTTTTCAAATTTCTGTTTCTGCATTTGTGCCACACCATCTCATCTCTTGCTGAAATGTTT
TTGTTAAATTAATTGCTTGATAAATTGCTAAGTACTTTTCATCAGACCAATTAGGACAATAGTAAGTATCCATCT
GTGGAGCGCGGACATTCAAGAAATCTGATCCAGTATTTAGAAAGTCATTCCTGAGCTGAGTTGGCTCAAACTGGC
ACCTTCTGGCATTTGCTTGTGGGTGGGGAATGTGGAATGCTTTGAAAGCTGAATGAGTTTGTCAAGTTTTAAAAT
TCCCTTATGGCTAAAGGAAAACAACATTCATTGTTTAAAAACACCATTGTTTGTTTTTTCTGCTTTTTTGTTCTT
TGGAGCCTGAATCTGCAAAAACACTCACACCCAGCATTTTGCTTCATGTACCACTCCTAAGATGTTTTTAGAGAC
TTGAATAGTGTCTCCGCACTACTTTTTATTGTGATTGTTCAGAATGTTCATAACAAATGGTAAAAAGTCAGTTTT
AGTGCTCAAATTGAGTTTTATGGAGAAAGACCATAATTTATGTTTGTCATTGTAAATTGATAGGAGAATTTTTGG
AAGTTTGCGTCCTAGAACCAGATTTCCAAGGCTCAGATCCTTATTTTCTCACTTCCTAGCTGTGTGACCTTAGAC
AAGGTATTAAACCTGTCTGTGCTGCCTCAGTGTCCTCATCTATTCTTTAAGAGTAAGAATAGAACCTACCCGATA
GAGTCACTTGAAGATTAAGTGGGTTAGTAAATTCAGAATGCTTGGAACAGTAACTAGCACAGAATAAGTGTCCAA
TAAAATTGGGTTGCAGCTATTATCAGTATTATTCCTGTCATAATCATCATCACCATTAAGCAATTAAATGTAGAG
TTCCAAAATTTGATTATGAAACTACAGTTATACAGCCATGATTCCCGGTGATACCACGTCAGTAACAAGATTATT
TCCTTAGCTTGAGCCAGTCACTACCTCATTGCATGTGGCAGAGTGTGTTGCCGTAGGCAAATGTCATTGTAGGGA
ATGAAAAAAAAATTGCCTGTGAGCTGCTCTCCAGAGGCCTCATCCCATTTTCCCATCGTCCACTTTACTCCATCT
CCACTGCCACTATTAGGACCTTATCATTTCTTGTCTAGATTAATTCAACAGCTTCCTTCCTTCTAGTCTCCATGA
TTTCACCCACTAGCCATCCCCTCCCCTTTGCCCAATTTTCTCCATTTATGGTAGAGTGATCTTTCTAATAGGAAA
CTCCTGACTTGCCTTAAAAAGCCCTCATTGAGGCCGGACGTGGTGGCTCATGCCTGTAATCCCAGCACTTTGGGA
GGCCGAGGCAGGTGGATCACGAGGTCAAGAGATTGAGACCATCGTGACTAACACAGTGAAACCCCATCTGTACTA
AAAATACAAGAAATTAGCCAGGCGTGGTGGCGGGTGCCTGTAGTCGCAGCTACTTGGGAGGCTGAGGCAGGAGAA
TGGCGTGAACCCGGGAGGCAGAGCTTGCAGTGAGCCGAGATTGCGCCACTGCACTCCAGCCTGGGCGACAGAGTG
AGACTCCGTCTCAAAAAAAAAAAGCCCTCATTGACAACCTTCAACCCACAATCCATGGTGAAGCACAGGAGCCTT
GGGGATCTGCCCCCAGCACACCTCTCCACCCTTGTCTCTCACTGCTCCTGCCTTCATGGAGAGCCCTGATGAACT
ATTTGTAGTTTCCCCTGACTCACCTTGCTGTTACTGGGCCTGTGTGCGTGTTGCTCCCACTACCTGCAATACGCT
TACCCACTTCACCTGGGTGAACTTTACTTAGGATTCACCTTAGGTGGGCATCATGTTCTTCCAGGCCCCTCCTCT
AACTTTTAGTTGAGAGTATTCCAGACTTAAGGCTCCATGGGATAGGGATCTTGTCTATGCACCAGCTTATTCCCA
ACTGCCTGGCACGTAATGCATTTATTAAATATATATTGAATTGATTACCCTACTTGGGGCTCTTGTTTGCTTCTA
CACTTACAGTTCTAGCATAGCACTTAACTCATTATCATGCATCATTATTATGGGTTTGTTTTGTCTCCCATTAGA
CTGTGAGCTCCACAAGGCTGTGTCCTTGTCTTATACATCATTGTATTTCCAGCTTCCAACATAGTGCTTGCCATG
ACACAGGAAGTCAGTAAGCTCTGAATGAATGAATAGTATCTACATACCATTAATCTGAGGTTTAAAGTTTCCCCA
AATTCTGAAGCAAGGGGATTTACGGACTTCCCTGACAATTTTTGGATGTCATCCCAATGATACCACTAACATTTT
AAGGGACAGCTTGCATATATACATTTTTCTGGATGGCAGTTTTTTTTCCCACAGGCTTCATCAGATATTTCTCCA
TAGCCTTCCTCAGATTCTCAAAGGGGTCTCTGATTCCCCCAAAAGATAAGAAACTGTCATAAAAAATTATTTCTA
AATATCAATTGTTAAATAAAATGTTTGCAAAGCAGCCTGATGAATCATTTCAGGCCACTTGACCCCGATGAGTTA
GAGAGTTTGTGCTCTGCAATCTGACTGCTTCCAGCAGTCTCACTGCTGCTGGACTGTGGCACTTCCAATTGGCAG
CAGGGCAAGTTTCTTCTGGATGAATATTCTGTCATAGGGGTCCCCCTTCCACACATACCTGTAGGAGCAGTTTGA
AACTCATATGCATGGTCTTCCTGGTTCTAGGCACATGAGTCATTTAAGCTGCTGGAGCCAGGACCAGCTAGTATG
CTAGCCCGGCATTCAGAAAGTTAAAATTTGGGGTCAAAACTGAGAACCTTCTTTGATCCACCTTGGCCAGACATT
TTCTCTGGCTTCCATTAATAGCCTCAACATTTTTTTTTTTTCTGGCCTAGACCCACACAGGCAAGAGACCAGAGC
TTCTCTAAGGAGCTAAGGGAAAGCACATTTTAAAAATAACTTGAGCAAATGAATTCATCTGGCAAAAGCAACCCC
ACTACGTAAAATAAACCTTTTTAGTTTCGCAATAGCAGTTCCTGAAAATGTAAACAACCTCAGGGTCTACATGCA
CTGAATCATTTGCTGAACAGAAAGTCCCTGGTCCAAATTCTGCAAGAATAAACACCTTACAAAACTAGGGGTCAA
TGACCTTCATATGGGAACAAGGAGGGTGTGGGGGGCAGCAACCCACCCTGAGGACAATGAGAAAGTCTTGAGACT
TGATATTCAAAATGCTGGCTTTCTAAACCAAAAACTGGCATGAGTGGAGGGAGAAGGGGAGGGTGGGCACAGTCT
ATGCCTCAGGCTCTTGCTCAGACCCTACCAGGCCCCTGCCTTCCCTAGGGAAAGCGAGAGTCTACTCACTGTCAT
GAAGCCAGAGGAAGGCCCTGCAGGTTTCACTGTGTGTTCTGTTGACAAGATGATGGTTCCATTGAAACTGTAATA
ACATACTTGGCCAACTAAGCCCATACGATCGTAGTAACTTTGTACCCAGTCCTAGCTTTTCAAACATAATGATAA
TATGTTCTTTCTAATGTGGCCCATACTGTTCTAATGAACTTATGCTGAGTTTTTCTGAGTACTAGAATAATATTC
GCCATAAATAATAGATATAATTATTCTCATTTAATATTTGCGTAGCTCTTCTTTAAAGCAGAAAGTATTTTCTCA
TTCCTTACTAGAACCTTTCTGTGTGAGGAGCACTGAGCTAGAACCCATATCTTAGAATGGTCAGAATTTGGAGAA
ATTCAGGGAAAAGGCACTGGACTCATTTTTAAAGACTAGAAAATGCAACCTCCAGAAAAAGATTCAAGAGTTTTT
TACTCCCAGAGATGTAGGAAAGATTGGAGTAAATCTTAATATTATATTTCAGGTAAACAAAGGATCACTGTCAAA
ATAGCAGCATTTATTGAGTAATGGCTGTGTGCCAGGTACTTTACAGTTTCACATTTAACCCTCATAATAACCTTG
TAAAGTGGATATCCCCTCAGTACATGATGAGAACACTGAAGCTTAGGTTAAATGATTGTCCAAATCGGACAATCA
TTTTCAAAATCTCCCCCTTTTTTTCTCCTTTCTTATCTGCAAGGCAGATTGCCCTTTCCCTTTCAGTGAAACTTG
TGCATGACCACATGACTCTCTTTGGCCAATGAAACATGAACAAGCAGCGTTTATCACTTTCAGATGGAAGGCTTT
GCATGAGCTTTGCCTCCTTTTCACTCTGCCACAGTGGCCACTAACATTCCAGATAGTGGCGCTCTGCAGGCTAGG
TCCTATAGTGGGAGCTATGGGCAGAGCCCCCTTTCCCACCCCCATCAAGATGTGCATGCTGCATAAGCCATGCAT
TAATCTTTGCAGTTTTAAGCCACTAAGTTTTGGAGTTATATTAATCATTAATCATGGTTCTCAAGAGAAACAGAG
TGGGGGAGTGGTATTCATTATGGGAATTGGCTTACATGATTATGGAAGCTGAGTAGTCCCCCAGTCTGCTGTTTT
TGAGCTGGAGAACTAGAGGAGCCAGTGGTATAATTCAGCCCAAGCCTGAAGGCCTGAGAAATGGGATGGGGGAAT
TGGGAGGGTGGGTGTGCTAGGGTAGGATAAGTCCTGAAGTTCAAAGGCCAGCCAGAAGGTGGATGTTTCAGCACC
AGAAGAGAGAGCAAATTCGCTTTTCTTCTGCCTTTTTGTCCTCTCTGGGCCCTCAATGGATTGGATGATGCCCTC
CCACATTGGTAAGGGTGGATCTTCTATACTCAGTCTGCTAATTTCTTCCAGAAACATCTTCACAGACACATCCAG
AAATAATGTTTTACCAGCTATCTCGGTATCCCTTAGCCTAGTCCATATTTAAAAATTAATGATCACAAGCAGTTG
TTTGTTTCCACAGCAAAACCTGGGTGACAGACCAAGTGACCCAGATGACTAGAATTTGACCTTCTTTTGTTGCCC
ACACCATACTCTGAACTAACATGCTGTGCTGCCTTCCAAGTGGAGAATGATGGCTAAGTATCTTCTACCTAATTT
GAGTCACAGAAAAAAAAAAAAAAGGTTATTAACTGCAGTGACAAGAATTGTGATTCCCCAGGGGGCAGATCAAGA
CTGATAGATAAGAGAAGTGAGGAACATCTGGGGAATGTCCATTGAAAATTTACTCAGAAGAGAAGAATAATTAAT
ATAATAATATGATATATTGAATTATAATAAATAATATTTTGATGTATTTCCTTCCAGGCATGTTTAAGTTATAGA
CTTTGAGTATATTTTCTCAAAGGGGGTTCTATGTAAGAGACTATTTCTTAATATAGTTCCTAGCTTGGAATTGCT
CTTGCTGGTTTAAGCTGAGCTTATTTTATTACAGACTTCACAACAATAACGTTTTCCTTCACTAGTCAGTACACA
AGATGGTCTTCATTTCCAGTTTGGAATCCCACACTATCAGAGCCTGAGACAAGGACTAGTATGCAGTTAGTTTGT
TTGGGAGGTGATTCCAGGAAGTGGGAATGAGAGATCAGTCAGCCTGCAACACGAAGGAGGAAAAGTCAATATAAG
GATGAATTTGGCAATTGGCCGTTTCATGCAACTGGGGCTAAATTTTGCTTGGCTCTCTAAGAAATGTAAAGAATG
CCTCCCGTAATTGCTCACCTCAAGTATTTATTCATTGGCTCTCATGCTCCATTGGTTGTCCATGAGAACTTTAGC
CCTCCCTCGCTGCAGCACAGACACTGTGCTTTCTCCTAGGCTGAGCAAGCTCCTGCATCTGTGGAAACCGTCCCG
GGGCAGATAGTGAAATAATGACTGCTGCGTGCTTGAGATCTGGGAAAGAGGCCACATCATAAGTGCACTGAAATC
AGAGATGTGTCAAGAGATGTGACACAGGGCATCTGAGGTGTCTACTGCACCAGCTATAACTCCCTAAACGCTAAT
CTCAGTTCTTACAGAGGGGATGGATGCAAGGGAACAGTCATGATTGAGAGCACCGAAGAAGCTCTGTATGAACCT
TAGGCAAGTTTCCTAATCTCCAAAATGAAGGTAATAATACCCACCATCCAAGATCTTCGGGAGGAATAGATGAAC
TAATGTATGTGAAAATGTCCAGCACAGGTCCTAACCCATAGTAGGTGCTCACCAAATGTTAGTTCCCTGCCCTCC
ACGTTGTGTGTATCCGGAGCTGCACTAGATGCTGAGGCAAATGGTCTCAAATGTACTTTAACACTTAATGACTGA
GATTTTTTCTGAGCTGCCTACAGGTTATTGACTATATTCATTATTAATAATAATATATATGGCCACTTCAGGCAA
CTGGGGCTAAATTTTGCTTGGCTCTCTAAGAAATGTAAAGAATGCCTCCTGTAATTGCTCACCTCAAGTATTTAT
TCATTGGCTCTCGTGCTTTATTGGTTGTCCCTGAGGACTTTAGCCCTCTCTCACTGCAGCACAGACACTGTGCTT
TCTCCTAGTTTCTGTGGCAAGTGACAGGAGCCCACCTCAAACTAAAGCAAAAGGGACTTCATTGGCTCTTGTAGC
TAGGAATTCCAGGGTTGGCACTGGCTTTGGGCACTACTGGATGCAGGAATTCAAACAATGTCTTCAACTCTTTCT
TTTGGTGTTTCTCTCAGCTGTGCTTCTCTTGTCGTTTCTTTTTCCCATTTTACAGATAAGTTCATCCGTAACTGA
GAGAGGTGAAAAGGGGATGGCTGCAGAGAACTCTGGCTTATATCATCCTTGCTTGCTGACCTCAAGGTCCATGTA
TAAATTCTCAGAGAAGAAGCCCTCTGGTTGGTGATGCTTGGAACATGCCCTGGAGGGTGGGCCCCTTGAAGTGGA
GCTTGCTGGAACCACATGGGCTGGAGCAAGGCGCTAGGGCCAGAAGAGAGAGGTAGGCAGGGCTGCTGGCCAGGC
ACTCTTCACCAAGACAAGGCAAGAGGAGGGGCATGATTGAGGCAGTGATACAGAAAGCAGACAGTAGAGGTCGTG
GCAAGTGTGCCGTTACTTGCTACCTGTGGTTGATGGGAGAGTCACACCACATTTAGGAGGAGAGAATCCATTTGC
CACTTCTGACAATGCCACAAGAATCACATATTTCATCCAGAGGTTGAATTTGGCCCATGCTGAGCTTTAAAATAC
AGAGCTGTCTTGGAACAATGGCTCAGTACATTCATTTGGTGTCCAACAAAGCCTGCCTCTGTTGCCTTCCCTCTC
TCTGTGTGCCCTTCAAGATCTTCATTGTGCTTTGGGGAGAGAAAGAGAAAATGTCATATCAGGGTAGCTCACCCC
ATGTGTCCTGGACTCAGGAAAAGAGTATCTTATCACCTTACTCTTTTGTTATTATAAAAAATAAAGTTGAACGTC
TTCAAATAAAATAAAGAAGTATAGAAAAAATTTTAAATTAACCTGTTATGATTCTACCTAGAGAACCATTGTCAA
CATCTTGGTATATGTACTTCCAGATACTTTCCTATGAATATATACATTGTAGATTTTTTAATATTAAAAGGCTAT
CATGCTGCTTTGTATACAGGCTTTCTTTACTGATATGTAATATAATACACAGACAAATATACAAATCCTAAGCCA
TCAACTCATTGAATTTTTATTCATTGTTTTTAATACCTGCATTGTGTTCCATTGTTAGGCTATGTCACAACATAT
TTAATTAAGCCCCTATTGATGAATATTAATTTACTCTATTTGCCAGTTCATTCCAGTCCAACATTTATTGAGTGT
CTACTTACGGGCCAGGCACTCTTGTATTCATCAAGATCACCACATTATCTGTATCAGTTATTTATTGCCACAATA
AAACTGCATAACAAATCACTCCAAAATGTAGCACCTTAAAACTACAACTACTTATTATTTCTCAAGAGTCAATGG
GTCAGCTGAGCAGTTCTGCCGATAGGGGTCAAGGTCAACACATTTCAACTAGACTACTTGTAAAAAAGAATGAGT
GTCTGGGTAGGTGTGTTCTTCTAAAAATAAAACAAGGAATGAGGAAATTGCAGGTAGGATAAGAGGGGTGGTTGG
CAACCAAACCCCACAAAAGGCAGACAAATTTTAAGGAAACATAATGCCAGACTCCTATGTCATCATCCAAGTAGA
TGCAGTGAAGTATAACCTGGGGCGTAGTAGGGTAGGAGTGGGGAGAGCAGAGGAGAAGGAAGGGAGATTGCTTTT
CATCACTTTTGGATTCCCTAATAACAGACATGACTGCCAGTATTAAAATTTAACAAAGGATATCTGATCATTAAT
TTTCCTGTATAAGTCACTGGTGATCTTCAACATCTCTCCCTCCCTTCCTCCCTTCCTTCCTCCCACCCTCCCTTC
CTTCCTTCTTTCCTCTTTTGCTTTCAACTTCCTTTTCTCGTTTCCTTTTGCTTTCTTTCTCTTCTCCCTTTTTTC
TGTCACTCTGGGCGTATGTAGTAGTGTAAAAAGGTTGACAGAGAAATCAAATATAACAGGAGCAGGGCCCTGAGA
AAAGCACCTGGCATCCTGTAGGCAAACCATTGTTTCTAAAAGAAGGGACTGAGAGATTGAGGAGCTCAGGACATT
GCCAAATGAACAAGGCAAGCACATTTATTCAGTACCAAACAAACGGAAAACGGCCTTTCCAAATAACTGACCTAT
AAAACAGCCTTTTCACAAGAGTACCGTAATTACTGGCCAACAGCAACAATGAAAAACAACTCCCAAACAAAGAAA
TATTTCTGGATTAAAAGCCATGAGATCTGGATTCTAACAAGCTGTGCTCCTCAAACTACAAGTACAAAATCTGGC
TCTAAACTAACAAGCTATGAGCCTCAAACTGATGACTGGCATGTTTGGGTCTCCATCTCCTTCTTGGGGGTTGGG
GTCTTAGAGACCCTTTTCCACGCCCTGATTCTCTTACTAGTGTGTATGCTTTCCTTTTGACTTCTCATGCTGACC
GTCTGAGCAGGAGTGAGAAGCAATTTCAAAGGAAAACATCGTTTATCATCTGCTGAAAGAAACCAAAAAGAACAC
AGGAAAACAAAAAGACAAGGAAAGGGAATGAAAATGTAATTCATTTTATTAAAAAGAAGAATTATTCTTCTGGGA
CACTGGATAGAAACCTTAATGAGTTACCTAGCTATCATAAATCCTCTAACAGAGAAGAGAAGAGAAAGAAACAAA
GACGGAAGAGGGCAGGATAAAAGAAAGAAAAAAGGAAGGGAAAAATGAAGGAAGGAAGTTATCTATTCATTTCTA
CAGAGACTCTGCTGAGCAGTAGACAAGAAGACTTGGGAAAAATTTAACTGAAACTTTTCCAAAAATCTTTTCAGA
GG
SEQ ID NO: 16
Reverse Comlement of SEQ ID NO: 15
CCTCTGAAAAGATTTTTGGAAAAGTTTCAGTTAAATTTTTCCCAAGTCTTCTTGTCTACTGCTCAGCAGAGTCTC
TGTAGAAATGAATAGATAACTTCCTTCCTTCATTTTTCCCTTCCTTTTTTCTTTCTTTTATCCTGCCCTCTTCCG
TCTTTGTTTCTTTCTCTTCTCTTCTCTGTTAGAGGATTTATGATAGCTAGGTAACTCATTAAGGTTTCTATCCAG
TGTCCCAGAAGAATAATTCTTCTTTTTAATAAAATGAATTACATTTTCATTCCCTTTCCTTGTCTTTTTGTTTTC
CTGTGTTCTTTTTGGTTTCTTTCAGCAGATGATAAACGATGTTTTCCTTTGAAATTGCTTCTCACTCCTGCTCAG
ACGGTCAGCATGAGAAGTCAAAAGGAAAGCATACACACTAGTAAGAGAATCAGGGCGTGGAAAAGGGTCTCTAAG
ACCCCAACCCCCAAGAAGGAGATGGAGACCCAAACATGCCAGTCATCAGTTTGAGGCTCATAGCTTGTTAGTTTA
GAGCCAGATTTTGTACTTGTAGTTTGAGGAGCACAGCTTGTTAGAATCCAGATCTCATGGCTTTTAATCCAGAAA
TATTTCTTTGTTTGGGAGTTGTTTTTCATTGTTGCTGTTGGCCAGTAATTACGGTACTCTTGTGAAAAGGCTGTT
TTATAGGTCAGTTATTTGGAAAGGCCGTTTTCCGTTTGTTTGGTACTGAATAAATGTGCTTGCCTTGTTCATTTG
GCAATGTCCTGAGCTCCTCAATCTCTCAGTCCCTTCTTTTAGAAACAATGGTTTGCCTACAGGATGCCAGGTGCT
TTTCTCAGGGCCCTGCTCCTGTTATATTTGATTTCTCTGTCAACCTTTTTACACTACTACATACGCCCAGAGTGA
CAGAAAAAAGGGAGAAGAGAAAGAAAGCAAAAGGAAACGAGAAAAGGAAGTTGAAAGCAAAAGAGGAAAGAAGGA
AGGAAGGGAGGGTGGGAGGAAGGAAGGGAGGAAGGGAGGGAGAGATGTTGAAGATCACCAGTGACTTATACAGGA
AAATTAATGATCAGATATCCTTTGTTAAATTTTAATACTGGCAGTCATGTCTGTTATTAGGGAATCCAAAAGTGA
TGAAAAGCAATCTCCCTTCCTTCTCCTCTGCTCTCCCCACTCCTACCCTACTACGCCCCAGGTTATACTTCACTG
CATCTACTTGGATGATGACATAGGAGTCTGGCATTATGTTTCCTTAAAATTTGTCTGCCTTTTGTGGGGTTTGGT
TGCCAACCACCCCTCTTATCCTACCTGCAATTTCCTCATTCCTTGTTTTATTTTTAGAAGAACACACCTACCCAG
ACACTCATTCTTTTTTACAAGTAGTCTAGTTGAAATGTGTTGACCTTGACCCCTATCGGCAGAACTGCTCAGCTG
ACCCATTGACTCTTGAGAAATAATAAGTAGTTGTAGTTTTAAGGTGCTACATTTTGGAGTGATTTGTTATGCAGT
TTTATTGTGGCAATAAATAACTGATACAGATAATGTGGTGATCTTGATGAATACAAGAGTGCCTGGCCCGTAAGT
AGACACTCAATAAATGTTGGACTGGAATGAACTGGCAAATAGAGTAAATTAATATTCATCAATAGGGGCTTAATT
AAATATGTTGTGACATAGCCTAACAATGGAACACAATGCAGGTATTAAAAACAATGAATAAAAATTCAATGAGTT
GATGGCTTAGGATTTGTATATTTGTCTGTGTATTATATTACATATCAGTAAAGAAAGCCTGTATACAAAGCAGCA
TGATAGCCTTTTAATATTAAAAAATCTACAATGTATATATTCATAGGAAAGTATCTGGAAGTACATATACCAAGA
TGTTGACAATGGTTCTCTAGGTAGAATCATAACAGGTTAATTTAAAATTTTTTCTATACTTCTTTATTTTATTTG
AAGACGTTCAACTTTATTTTTTATAATAACAAAAGAGTAAGGTGATAAGATACTCTTTTCCTGAGTCCAGGACAC
ATGGGGTGAGCTACCCTGATATGACATTTTCTCTTTCTCTCCCCAAAGCACAATGAAGATCTTGAAGGGCACACA
GAGAGAGGGAAGGCAACAGAGGCAGGCTTTGTTGGACACCAAATGAATGTACTGAGCCATTGTTCCAAGACAGCT
CTGTATTTTAAAGCTCAGCATGGGCCAAATTCAACCTCTGGATGAAATATGTGATTCTTGTGGCATTGTCAGAAG
TGGCAAATGGATTCTCTCCTCCTAAATGTGGTGTGACTCTCCCATCAACCACAGGTAGCAAGTAACGGCACACTT
GCCACGACCTCTACTGTCTGCTTTCTGTATCACTGCCTCAATCATGCCCCTCCTCTTGCCTTGTCTTGGTGAAGA
GTGCCTGGCCAGCAGCCCTGCCTACCTCTCTCTTCTGGCCCTAGCGCCTTGCTCCAGCCCATGTGGTTCCAGCAA
GCTCCACTTCAAGGGGCCCACCCTCCAGGGCATGTTCCAAGCATCACCAACCAGAGGGCTTCTTCTCTGAGAATT
TATACATGGACCTTGAGGTCAGCAAGCAAGGATGATATAAGCCAGAGTTCTCTGCAGCCATCCCCTTTTCACCTC
TCTCAGTTACGGATGAACTTATCTGTAAAATGGGAAAAAGAAACGACAAGAGAAGCACAGCTGAGAGAAACACCA
AAAGAAAGAGTTGAAGACATTGTTTGAATTCCTGCATCCAGTAGTGCCCAAAGCCAGTGCCAACCCTGGAATTCC
TAGCTACAAGAGCCAATGAAGTCCCTTTTGCTTTAGTTTGAGGTGGGCTCCTGTCACTTGCCACAGAAACTAGGA
GAAAGCACAGTGTCTGTGCTGCAGTGAGAGAGGGCTAAAGTCCTCAGGGACAACCAATAAAGCACGAGAGCCAAT
GAATAAATACTTGAGGTGAGCAATTACAGGAGGCATTCTTTACATTTCTTAGAGAGCCAAGCAAAATTTAGCCCC
AGTTGCCTGAAGTGGCCATATATATTATTATTAATAATGAATATAGTCAATAACCTGTAGGCAGCTCAGAAAAAA
TCTCAGTCATTAAGTGTTAAAGTACATTTGAGACCATTTGCCTCAGCATCTAGTGCAGCTCCGGATACACACAAC
GTGGAGGGCAGGGAACTAACATTTGGTGAGCACCTACTATGGGTTAGGACCTGTGCTGGACATTTTCACATACAT
TAGTTCATCTATTCCTCCCGAAGATCTTGGATGGTGGGTATTATTACCTTCATTTTGGAGATTAGGAAACTTGCC
TAAGGTTCATACAGAGCTTCTTCGGTGCTCTCAATCATGACTGTTCCCTTGCATCCATCCCCTCTGTAAGAACTG
AGATTAGCGTTTAGGGAGTTATAGCTGGTGCAGTAGACACCTCAGATGCCCTGTGTCACATCTCTTGACACATCT
CTGATTTCAGTGCACTTATGATGTGGCCTCTTTCCCAGATCTCAAGCACGCAGCAGTCATTATTTCACTATCTGC
CCCGGGACGGTTTCCACAGATGCAGGAGCTTGCTCAGCCTAGGAGAAAGCACAGTGTCTGTGCTGCAGCGAGGGA
GGGCTAAAGTTCTCATGGACAACCAATGGAGCATGAGAGCCAATGAATAAATACTTGAGGTGAGCAATTACGGGA
GGCATTCTTTACATTTCTTAGAGAGCCAAGCAAAATTTAGCCCCAGTTGCATGAAACGGCCAATTGCCAAATTCA
TCCTTATATTGACTTTTCCTCCTTCGTGTTGCAGGCTGACTGATCTCTCATTCCCACTTCCTGGAATCACCTCCC
AAACAAACTAACTGCATACTAGTCCTTGTCTCAGGCTCTGATAGTGTGGGATTCCAAACTGGAAATGAAGACCAT
CTTGTGTACTGACTAGTGAAGGAAAACGTTATTGTTGTGAAGTCTGTAATAAAATAAGCTCAGCTTAAACCAGCA
AGAGCAATTCCAAGCTAGGAACTATATTAAGAAATAGTCTCTTACATAGAACCCCCTTTGAGAAAATATACTCAA
AGTCTATAACTTAAACATGCCTGGAAGGAAATACATCAAAATATTATTTATTATAATTCAATATATCATATTATT
ATATTAATTATTCTTCTCTTCTGAGTAAATTTTCAATGGACATTCCCCAGATGTTCCTCACTTCTCTTATCTATC
AGTCTTGATCTGCCCCCTGGGGAATCACAATTCTTGTCACTGCAGTTAATAACCTTTTTTTTTTTTTTCTGTGAC
TCAAATTAGGTAGAAGATACTTAGCCATCATTCTCCACTTGGAAGGCAGCACAGCATGTTAGTTCAGAGTATGGT
GTGGGCAACAAAAGAAGGTCAAATTCTAGTCATCTGGGTCACTTGGTCTGTCACCCAGGTTTTGCTGTGGAAACA
AACAACTGCTTGTGATCATTAATTTTTAAATATGGACTAGGCTAAGGGATACCGAGATAGCTGGTAAAACATTAT
TTCTGGATGTGTCTGTGAAGATGTTTCTGGAAGAAATTAGCAGACTGAGTATAGAAGATCCACCCTTACCAATGT
GGGAGGGCATCATCCAATCCATTGAGGGCCCAGAGAGGACAAAAAGGCAGAAGAAAAGCGAATTTGCTCTCTCTT
CTGGTGCTGAAACATCCACCTTCTGGCTGGCCTTTGAACTTCAGGACTTATCCTACCCTAGCACACCCACCCTCC
CAATTCCCCCATCCCATTTCTCAGGCCTTCAGGCTTGGGCTGAATTATACCACTGGCTCCTCTAGTTCTCCAGCT
CAAAAACAGCAGACTGGGGGACTACTCAGCTTCCATAATCATGTAAGCCAATTCCCATAATGAATACCACTCCCC
CACTCTGTTTCTCTTGAGAACCATGATTAATGATTAATATAACTCCAAAACTTAGTGGCTTAAAACTGCAAAGAT
TAATGCATGGCTTATGCAGCATGCACATCTTGATGGGGGTGGGAAAGGGGGCTCTGCCCATAGCTCCCACTATAG
GACCTAGCCTGCAGAGCGCCACTATCTGGAATGTTAGTGGCCACTGTGGCAGAGTGAAAAGGAGGCAAAGCTCAT
GCAAAGCCTTCCATCTGAAAGTGATAAACGCTGCTTGTTCATGTTTCATTGGCCAAAGAGAGTCATGTGGTCATG
CACAAGTTTCACTGAAAGGGAAAGGGCAATCTGCCTTGCAGATAAGAAAGGAGAAAAAAAGGGGGAGATTTTGAA
AATGATTGTCCGATTTGGACAATCATTTAACCTAAGCTTCAGTGTTCTCATCATGTACTGAGGGGATATCCACTT
TACAAGGTTATTATGAGGGTTAAATGTGAAACTGTAAAGTACCTGGCACACAGCCATTACTCAATAAATGCTGCT
ATTTTGACAGTGATCCTTTGTTTACCTGAAATATAATATTAAGATTTACTCCAATCTTTCCTACATCTCTGGGAG
TAAAAAACTCTTGAATCTTTTTCTGGAGGTTGCATTTTCTAGTCTTTAAAAATGAGTCCAGTGCCTTTTCCCTGA
ATTTCTCCAAATTCTGACCATTCTAAGATATGGGTTCTAGCTCAGTGCTCCTCACACAGAAAGGTTCTAGTAAGG
AATGAGAAAATACTTTCTGCTTTAAAGAAGAGCTACGCAAATATTAAATGAGAATAATTATATCTATTATTTATG
GCGAATATTATTCTAGTACTCAGAAAAACTCAGCATAAGTTCATTAGAACAGTATGGGCCACATTAGAAAGAACA
TATTATCATTATGTTTGAAAAGCTAGGACTGGGTACAAAGTTACTACGATCGTATGGGCTTAGTTGGCCAAGTAT
GTTATTACAGTTTCAATGGAACCATCATCTTGTCAACAGAACACACAGTGAAACCTGCAGGGCCTTCCTCTGGCT
TCATGACAGTGAGTAGACTCTCGCTTTCCCTAGGGAAGGCAGGGGCCTGGTAGGGTCTGAGCAAGAGCCTGAGGC
ATAGACTGTGCCCACCCTCCCCTTCTCCCTCCACTCATGCCAGTTTTTGGTTTAGAAAGCCAGCATTTTGAATAT
CAAGTCTCAAGACTTTCTCATTGTCCTCAGGGTGGGTTGCTGCCCCCCACACCCTCCTTGTTCCCATATGAAGGT
CATTGACCCCTAGTTTTGTAAGGTGTTTATTCTTGCAGAATTTGGACCAGGGACTTTCTGTTCAGCAAATGATTC
AGTGCATGTAGACCCTGAGGTTGTTTACATTTTCAGGAACTGCTATTGCGAAACTAAAAAGGTTTATTTTACGTA
GTGGGGTTGCTTTTGCCAGATGAATTCATTTGCTCAAGTTATTTTTAAAATGTGCTTTCCCTTAGCTCCTTAGAG
AAGCTCTGGTCTCTTGCCTGTGTGGGTCTAGGCCAGAAAAAAAAAAAATGTTGAGGCTATTAATGGAAGCCAGAG
AAAATGTCTGGCCAAGGTGGATCAAAGAAGGTTCTCAGTTTTGACCCCAAATTTTAACTTTCTGAATGCCGGGCT
AGCATACTAGCTGGTCCTGGCTCCAGCAGCTTAAATGACTCATGTGCCTAGAACCAGGAAGACCATGCATATGAG
TTTCAAACTGCTCCTACAGGTATGTGTGGAAGGGGGACCCCTATGACAGAATATTCATCCAGAAGAAACTTGCCC
TGCTGCCAATTGGAAGTGCCACAGTCCAGCAGCAGTGAGACTGCTGGAAGCAGTCAGATTGCAGAGCACAAACTC
TCTAACTCATCGGGGTCAAGTGGCCTGAAATGATTCATCAGGCTGCTTTGCAAACATTTTATTTAACAATTGATA
TTTAGAAATAATTTTTTATGACAGTTTCTTATCTTTTGGGGGAATCAGAGACCCCTTTGAGAATCTGAGGAAGGC
TATGGAGAAATATCTGATGAAGCCTGTGGGAAAAAAAACTGCCATCCAGAAAAATGTATATATGCAAGCTGTCCC
TTAAAATGTTAGTGGTATCATTGGGATGACATCCAAAAATTGTCAGGGAAGTCCGTAAATCCCCTTGCTTCAGAA
TTTGGGGAAACTTTAAACCTCAGATTAATGGTATGTAGATACTATTCATTCATTCAGAGCTTACTGACTTCCTGT
GTCATGGCAAGCACTATGTTGGAAGCTGGAAATACAATGATGTATAAGACAAGGACACAGCCTTGTGGAGCTCAC
AGTCTAATGGGAGACAAAACAAACCCATAATAATGATGCATGATAATGAGTTAAGTGCTATGCTAGAACTGTAAG
TGTAGAAGCAAACAAGAGCCCCAAGTAGGGTAATCAATTCAATATATATTTAATAAATGCATTACGTGCCAGGCA
GTTGGGAATAAGCTGGTGCATAGACAAGATCCCTATCCCATGGAGCCTTAAGTCTGGAATACTCTCAACTAAAAG
TTAGAGGAGGGGCCTGGAAGAACATGATGCCCACCTAAGGTGAATCCTAAGTAAAGTTCACCCAGGTGAAGTGGG
TAAGCGTATTGCAGGTAGTGGGAGCAACACGCACACAGGCCCAGTAACAGCAAGGTGAGTCAGGGGAAACTACAA
ATAGTTCATCAGGGCTCTCCATGAAGGCAGGAGCAGTGAGAGACAAGGGTGGAGAGGTGTGCTGGGGGCAGATCC
CCAAGGCTCCTGTGCTTCACCATGGATTGTGGGTTGAAGGTTGTCAATGAGGGCTTTTTTTTTTTGAGACGGAGT
CTCACTCTGTCGCCCAGGCTGGAGTGCAGTGGCGCAATCTCGGCTCACTGCAAGCTCTGCCTCCCGGGTTCACGC
CATTCTCCTGCCTCAGCCTCCCAAGTAGCTGCGACTACAGGCACCCGCCACCACGCCTGGCTAATTTCTTGTATT
TTTAGTACAGATGGGGTTTCACTGTGTTAGTCACGATGGTCTCAATCTCTTGACCTCGTGATCCACCTGCCTCGG
CCTCCCAAAGTGCTGGGATTACAGGCATGAGCCACCACGTCCGGCCTCAATGAGGGCTTTTTAAGGCAAGTCAGG
AGTTTCCTATTAGAAAGATCACTCTACCATAAATGGAGAAAATTGGGCAAAGGGGAGGGGATGGCTAGTGGGTGA
AATCATGGAGACTAGAAGGAAGGAAGCTGTTGAATTAATCTAGACAAGAAATGATAAGGTCCTAATAGTGGCAGT
GGAGATGGAGTAAAGTGGACGATGGGAAAATGGGATGAGGCCTCTGGAGAGCAGCTCACAGGCAATTTTTTTTTC
ATTCCCTACAATGACATTTGCCTACGGCAACACACTCTGCCACATGCAATGAGGTAGTGACTGGCTCAAGCTAAG
GAAATAATCTTGTTACTGACGTGGTATCACCGGGAATCATGGCTGTATAACTGTAGTTTCATAATCAAATTTTGG
AACTCTACATTTAATTGCTTAATGGTGATGATGATTATGACAGGAATAATACTGATAATAGCTGCAACCCAATTT
TATTGGACACTTATTCTGTGCTAGTTACTGTTCCAAGCATTCTGAATTTACTAACCCACTTAATCTTCAAGTGAC
TCTATCGGGTAGGTTCTATTCTTACTCTTAAAGAATAGATGAGGACACTGAGGCAGCACAGACAGGTTTAATACC
TTGTCTAAGGTCACACAGCTAGGAAGTGAGAAAATAAGGATCTGAGCCTTGGAAATCTGGTTCTAGGACGCAAAC
TTCCAAAAATTCTCCTATCAATTTACAATGACAAACATAAATTATGGTCTTTCTCCATAAAACTCAATTTGAGCA
CTAAAACTGACTTTTTACCATTTGTTATGAACATTCTGAACAATCACAATAAAAAGTAGTGCGGAGACACTATTC
AAGTCTCTAAAAACATCTTAGGAGTGGTACATGAAGCAAAATGCTGGGTGTGAGTGTTTTTGCAGATTCAGGCTC
CAAAGAACAAAAAAGCAGAAAAAACAAACAATGGTGTTTTTAAACAATGAATGTTGTTTTCCTTTAGCCATAAGG
GAATTTTAAAACTTGACAAACTCATTCAGCTTTCAAAGCATTCCACATTCCCCACCCACAAGCAAATGCCAGAAG
GTGCCAGTTTGAGCCAACTCAGCTCAGGAATGACTTTCTAAATACTGGATCAGATTTCTTGAATGTCCGCGCTCC
ACAGATGGATACTTACTATTGTCCTAATTGGTCTGATGAAAAGTACTTAGCAATTTATCAAGCAATTAATTTAAC
AAAAACATTTCAGCAAGAGATGAGATGGTGTGGCACAAATGCAGAAACAGAAATTTGAAAAGATTCAAATGTATT
ACTTCAAAAAGATTGAAAAATTGCTGCCTAAAATCTTTGAAACTTTAGAGCTAGGTTGTGCATAGGCTTAATTTT
TAATGAAGAATCTTCTGGGAAAATAAGTTTTTGATTATATAAAGAAAGCAATTTCCTTGATTTGCAGGAAATAAT
TCTTTTTGGGGAAAAACAAATAAATTAATATTTAATATACTCATTACATCCATCCAGCTAACATTTACTGGAAGC
ATGCTGTGTGCCTGGCACCATGCTAGGCACTGAGACACAAAAATGAAAAAGACAAGAATCTTGTTCTTTAGCCTA
GGTGGGGAGATAGATAAGCAAAGGAATAGTGTAATAAATGCAATAAAAGAAATACGTACTAGCTAGACCTGTGGG
AAGAAAGGTTAGAAAGATGAACAGAAACTGTGCTAGAATTTTGGACCTAATTCTGTAAGTGTTGGAGAGCCATTA
AGCAGAAGTGATCGTTAGATTTAATTTTAGAAGGATCATGCTGTTATCAATGTGAAGAATGGGCTGTAGTGGGGA
AACGCCAAAGGCAGATTCTGTTCCACTCTTCATAAAAATCGTACCACAGCACTCTGATACCCTTTTTGCTCTCTA
ATCCATTTCTCTTTTCCCCCCAACACAGATAATTTAGAAATTATTTAGTCTTTTAAAAGCGCTGGTGTCCAAGTT
TTTGAGGTTTTCCCAGATATTTGTTATTGGTTAATAATTTAATTCCATTGTGGTCAGAGAACATATTTTCTATGA
CTTGAATCCTTTTAAATTTATCAAGATTTGTTTCATGGCTCAAAATACGGTCTATCTTGGTACATGTTTCATGTA
CTCTTGGAAAGAATGTGTGATTTATTGTTGTTGGGGACTGCTGTGCTCTACAAATGTCATTTAAGTCAAATTGTT
TGATAGTCTTGTTCTAATCTTCTATATCCTTACTGATTTTGTGTATTCTTGTTCTACTAAACATTGAAAGGATAT
TAAAATCTCCAGGCTGCGTGCAGTGGCTCATGCCTGTAATCCCAGCACTTTGGGAGGCCGAGGCAGGCAGATCAC
GAGGTCAGGAGATCGAGACCATCCTGGCTAACACGGTGAAACCCCGTCTCTACTAAAAATACAAAAAAATTAGCC
AGGCGTGGTGGTGGCCGCCTGTAGTCCCAGCTACTGGGGAGGCTGAAGCAGGAGAATGGCGTGAACCTGGGAGGC
GGAGCTTGCAGTGAGCTGAGATCCTGCCACTGCACTCCAGCCTGGGCGACAAAAAAAAAAAAAAAAAAAATCTCC
AAACATAATTGTGGACTTGTCTACTTCTTACAGTTCTATCAGTTTTTGCTTCATCTATTTTGACACAGCTATGAG
GTGAAAAAACATTTAGGACTGTTATATCCTTTTGCTGAACTGACTCTTTTATCATTATAAAATGATCATTTTTAC
CCCTGGTAATATTCTTTGCTCTGAAATCTATTTTGTTTCATATTGTCACTCCAGTTTTCTTCTAATTACCAATAT
GATGTATCTTTTCTCCATCCTTTTACTTTTAATCTGTTTGTTCGTCCTTTATTTTTAAGGTGTGTTTCCTGTTGG
CAGCATATAGTTGGGTCTTGCTTTTTTATCTGATCTGAAAATCTGATTTTTTAAGGACATTTCTAAAATTGAAAA
ATAAAAGTTGTATATATTTATAGCATACAATATGATGTTTTGAAATAAGTATACATTGTGAAATGACTACAGTTA
ATTAATATATACATTACCTCACATATTTATTTGTAGAGAGAACACTTAAAAGATACTGTTAGCAATTTTCAAGTG
TACAATATATGTTGTTGGTTATAGTCACATTGTACAATAGATCTCTTGATCTTTAGCTTATTCCTCCTATCTAAC
TGAATAAGTTGTACAATAGATCTCTTGAACTTATTCCTCCTGTCTAACTTTGACCAACATCTCCCCAGCCCACCG
CCTCTCCCCAGCACCTGGTAACCATGTTTTTACTCTCTGCTTCTATGAGTTCCAAGTTTTTCAATTCTGTGTGTA
AGTGAGATCATGTGGTATTAACAATCTCTGACTTTTAATTGGGGTGTTTTGAATGTTTATATTTAAATATCTTTA
AAAGATAATTGACTGTATAAGTAAAATTAAAAAACAATAAAGTGTGGAGTTTATAATGTTTGTAGAAATAAAAAC
GTTAAGACCATAACAGCAAAAAGACAACAACAGTAAGTTTCTTAAGCTATATGTCAAGTAGTATATAAACTGGTT
ATAAACTGGTAAATTAGAAAGAGGTATAACCCTAAAACAACCACTAAAATAAATCAAAGAGTATAGTTAATAAGA
CAATAGAAAAGACATAGTAGAATAAAAAAATCCAAAAGTAAGGAGAAAACAAAAAGAACAAATAGAAAACAGCAA
GATGACAGCTTTAACCTACCATGCCAATAATCATATTAAATGTTCTAAATGCCCCAATTACAAGCTGGAGATTGT
CAGATTGGATGAAAAAGCATTTATGAAGCACTAGTCAGGTACTGAACTAAGGATCGTATAAGGTCTCATTAAATC
CTCTGGTGAAGTACGTATTATCACTCCATTTCCAATATTTACAAAATGAACCTTGCATAGGTTAAGTAACTTCTC
CAAGCAGCACAGCAGGGATTCACACTCGGGCTCTGAATAGTGCAAGTAGACCTGTAAAACCTGCTTCCTTACTCT
CCTTTCAATTCAGCTACACTGAGTACCAGTTACATGGAGTTACTAAGTTAGGAGCTGTGCCCATGGCAATGACTG
TAACTGTCTGGGATGAAATCCACCTTTACCAGCTGTGTGACCTTGGGCGGTGTATTACACATCTTTAAGCTTTAG
TTTCCTCATTTGTAAAATGGAAGTAATAAAACTAGTCCCATAGTGTTACTGTGTGTTAAATGGAGGTGGTTGACT
GTTTTTGTTACTTTAAGCTATACCCTGCAGATCATAGGAGAAATATCAATTTGTGCAATTCTACATCAAAATCAA
AATAGTATTATATAAGAGTGATCTGTTTAAACTGTAGCATTATAGAAATATACTATAGAAGGAACTATTTTATAT
TTACTTTATAGTATTATATGAAGTTTTGGTTTCTTCTTTGAGAATCATAGTAATGTGATCAGTTAGTCTTCTTTT
AAAGAAACCTCTCATCCCAACTATTCTATTTTCAAAAAAAAACTTTTTCTTGGAGTGAAAATGTCAAACCTATTC
CTAGCTTTGCAGATGAAAGTGTTCAGATTCCTTGGATTTTTACTTTGGATACATGTATTAGATACCTGGGATGAA
TAACTTCAGTTTAGGTTATATTAATATTATAATAATAAGAATGTTTAAAAACAATTTATCTAAATAGTATTTTAC
ATTTGAAATATTAATATATAATTTGAAAAATCAAAATGAAATCCAGAGAGTTAAGCAGAAAGTTTTTTTAACATA
AGATTATATTATAAAGCTTCTTTAAATATTTTTTCTTTGAAAAGACAAAATTAATTTTAAAAAAAGTTTAGATCC
AGCATGACTGTCAGCTGAACAGAAGCTGATCAGTCTTCAAATATCATAGTATTTTTTTCTGAATGAACAACTGAA
AGAGGCAAAATTTCAAATATATTTTATTCAAAATTCTCCATTTAGGAGAAAAGATATATAACAATGTTTACACAT
GCTTTAATAACTTATTTCACTGTACAACTTACATTCTGTATAACAGTACAATAAACCAGCCAAAGAAAATAACCA
GTTAGCACTTAAATAAGAATCTACCATGTAAAAAACACAGTATGGGACACTACAAGGTAGTATTTATATATTTTT
TAAATGACTGAGCTACAGTACAACAGTCATCTAGTTCAGTGGTTGTCTAAAACATCAAGCTGTCCACATCTTTCT
GATTCATGATGGGAAAGCTATTATGACCTTTCACATTCGAACATGTCATTTTGTTGTGTAAATTTGGTGGGTGGG
GGGCAGAAGGGCTCTATTACCTTTATCCCTTTCTTATAAATATATTTTCCCTTTTATATTACTTCCAGAATTTTA
AATAAAATATTTATTTGTGTTTGTGTAACTACAATGTCAACATTGTGTTAGAATTATATTAATCAGTTATTTTAT
AGCCAGCAAAATGGTCCAAACGCATTAAGAAAACAAAAGATAACTTAGCTCTGCCAAAGTAGCACCTAGGAAAAC
AGCACTTCAGTAAAATTTCTCCTGCTATTCCTTATAGCCTATTGCAGGCAAACAGCAACAACTTCAAAAACATAG
GCAGAAATGCAAGAAGGAAGTACTCAAGGAACTATTTTCTGTTTCTGAAAAAAAGATCTTATTAGTTAGTATATT
CTCTAAGGCATTTTATAATCTCAGAGTTGCAATGATTGCCAAAGCAGGTACATGGTAAGACTTAGCAAGAAGAAA
ACAAGATGAAAATGACTGACCAGTGAAATCTGAACTTGTTCCAAGTAATTAGATTTTCTAAGGAGAAAAAAGGCA
CAGGAGGTGCACATTTCAATTTGGCTCAAAAATAATGAAAATTAATTTAAAAAGTTGGCAACAATTACTAAAACA
TAAAATACAATTTCCTTTTACAGAGCTCAACTACATAGAATATCAACAATAGCAAGAACAATAAAATAAACAAAA
CACCCTAACTGTGGTTCACCAGGAACAAGGACTATAGAGAACTATTAGACATTTCTACAGACTGAATCCCAGGGA
CTAAATCCACAAAGTAGGATCTAATAATCAATTCCAATTAAACATCTGCTTGATCAATTTTCTTTCTAAAGCTCA
TCCTATGTTCAAGCTCACTTTCTTTCTGTGGAATTGAAATCATTTAAAGGCTCTAGTTTTCAGTTGATTGCAGAA
GTATTACAGTAATTCTACACACCAAAGAATGCCAAAAGATAGAAAGTCAACTATCTGTAAAAGCCAACTCAGATT
TCACCTTGACACATGTAAACCATGAATCATGTATTTCAAAAAAACAGTAGTTGTGGTCAAGTTTACATCCTATTA
TTATATCTTTAAAAGATAGCAATAATATTTATTATATTGTAAACATAGGTCTGTATCCCAAAAGCATAAATCTAG
GAAAAGAGACACCCAATGTAATGATGCACCTGACATCCCCTCACAGGCTCTTGTGAGAACTGTAGTGTAACTTAC
TTAACTGCAATTGCTGAGAGCAGAATTCTGGAGTATGATCCAGGGGAACGTTTCCCCACACCACTGAGCTACTTT
ACCAGCGATCATGATTGTGATGGAATAGGCTTATTAAGTTACACATTTAAAAAGTCATTAGAACATCTCGTTCTT
GCACACTAGTGTAGAAAGGTCTTCCAAAGATAAAAGAGTGTAGGCCTGGTTTAATTTTCTCAGCCAGAGCCATTA
TTATGTTAAGATCGCCCTCTGCTGTTAAATCAAGGTCTATCTTCAGGTTCCGAAGAGATTTAAAGGGCTTTTTTC
CCTTCTGCCTAAAAATAATGGAAAAAAAAAAAAAAAAAAAAAAAAAAGAAGCGCAAAAATTATGATATAGAAAAT
GTACAAAGGAAACAAAACAAAAAAACAATGTACAAAGGTTTTTCTTCCGTGTAGGAGTTTTACTCACGTATCGTC
TTCTATATATTTTATTAGTGTCAAGGCTTTTCTGTGAAGGACAAGTAGAAACTGTGCAAGGAAAGTACTTCTGAG
AGATAAGCCAGGTTTCAGCTGAAAGACCTGCAGGGAGAGGAACCATTTCAACACATAATTTGCTCACATTCTACT
CGTACAGAAGTTTCTATGACAGTGTTGACAGTGCTTGGCAGACAATACACACTAAACATCTCCTTAACCATTTCC
ACTGATGCCCCCTAACAGGAGTGTGGTATAGAAGCACTAGGTGTAGGAACAATCTGATATTTTTTTTTTTTTTTG
AGACGGAGTCTAGCTCTGTCACCAGGCTAAAGTGCAGTGGCGCGATCTTGACTCACTGCAACCTACACCTCCTGG
GTTCAAGCGATTCTCCTGCCTCAGCCTCCCAGGTAGCTGGGATTACAGGCACACGCTGCCACGCCCAGCTAATTT
TTGTATACTTAGTAGAGACGGGGTTTCACCATGTTGGCAGGATGGTCTCGATCTCCTGACCTTGTGATCCACCCA
CCTCGGACTCCCAAAGTGCTGGGATTACAGGCATAAGCCACCAGGCCCAGACAATCTGACAGTTTTTAGAAGTTT
TCTAATTTATCTTTAAAAATGCATTCAAATTTTGCATTCTACTTCAGAATACATCCATGTTTACTTCACTTTTAA
AACTCATACATAAGTTATTTCGTATTACTTAATTTTTTCTGCCCAAATCTTCAAATAACATTGAAGCTCCTAGAA
GATGAGCCTATGGTTTTGAGACATTCATATACCTGTACTTTTGCAGGGGGTGGAGTGGGTGGGTAGGAGTAGAAT
GTGTGCATATGATGAGGTGGAGAGTGGGAGGGATTTTATGTCAGCTTGAGAAGCATGAAATTCCAGGCCTACGAA
TTTTCCTTGAACTTCGCAATATGTCACAGACCTCATAGATCTCAATCCAAAATTTCTAGTAAACATAAGGATCTC
ATTATTTCAAAGCTTTATCTATGACACAGTTTCCTGGCCTGATCCCCAGAATGTTATTATAACAAACTGTAATAT
ATTAAGAAATAAGTTCCATATAATCTACGATTTCCTTCAACTGTGTAATTAAGTAGATATTTTTTAAAAGAATGA
AACAATAAAGATGTTATTTAAATAAGTACACTATGTTTCAAAAATATTTTTGAGTCATTTCAGGCAAATAAACAA
CCTCCCTTTCTCAAAAAAAAAAAAAAAACTTACTTGTTATAGGTTAATGCTTGAATTAAAATGTTTGTTGTTTGA
CGTATGGAATGCTGCACTCTTAATTGTTAATATACAGGCTCTTCCCCATTAATAAGTCAAAAGGAAAAATCATTT
AGACACTTTAGAAAGACCAGACACTACAAAAAGCACCTAATAAAACAGATTAGGACTCAATTCTTAATTCTATCA
TTTTATAAATGTTAAAATATATATAAAAAATATATTACACTCTATCATAGAGTATATGTTATATATTTTACATAT
CATATATAACATATACTCTACATAACATAATAGGGTTTTAAGTTTATAACTTTTTTTTAATATATAAACATATAT
ATATCCTACAATGCTGGTAATATGTATATATGCTCGTACTATATATATATACACACACAAACATATATATATACT
TGTACTACATATATAGTACAAGCATTGCAGGATATATATATGTATATATTATATCTGTAAAAGCTAAGTCAGATT
TCACTTTGATACATGTAAACCAGGAATTCTATGTTTCAAAAAACAGTAGTTGTGGTCAACTTTATATCTTATTAT
TATATCTTTAAAAGATAGCAATAATATTTATTATATTGTAAACAAAGGTCTGAATTCCCGAAGCATAAATCTAGG
ACAAGAGGCAACCAATGTAATGATGCACTTGACATGCCCTCACAGACTCTTCTGAGAACTGTAGTGTAACTAACT
TAACTGCAATTGCTGAGCAGAACTCTGGGGCATGATCCAGGGGAATATAAATATATAGAACAGGCATTGTAGGAT
ATATTAAAAAAGAAAACATGTCATATCATTCACTCTGACAATCTCAAGTTCAACATTTACCTGATCCAGGAAGGC
TTTCACTAGAGTGTCTCTGTGTAAGACATCTTGAAAAATATTCCTGAAGAAAAGAAGAAAATGAAGAAAAGAAAA
AAGTTCAAATGTTGAAATGGCCATATAATGCTACCTTCCACAATCCCTCACTTGAGCCAACATTTTAAGTTCTTA
TAAATTGATATTACATATAAACACATTTGTAAGAATATAGATATATATATTTGGAGACACAAAATATACATTCAT
TTATTTACTCAAGGATATATTTACATTTTACTGGTGTTTGTCAGGCTAATATGTAATTTTTTCTCTAAAGAATAT
AAGCAGTCCCCAGTAAGGGTATGTATGTATCTATCAATCTATTTTACTACTCTCATCTTTTAAAAAAAGTAGAGA
CCCTTAGGCAATTAATAAATAAAACTACCAATTCTTGCCATCTCAAAAAAAAACAAAAAACAAACCAGCAACAAA
AATAAGCCACTACCATCAAAAACCAAAACAAACCAAAAAATGTGAAGAAAAATTTGAGTGAGATTAGAGAACACA
CTCCGATGATTATCCACTGGGTTTGCCTCAACAAGCCCACCATCTGTAAGTAACATGAACCAACTATCCAAGAAA
GTACTACCTACCTTGGCAACGACCAGAGTGAGTAGTAGTAATATAATGGCCACTTAAAATAATGTGTGCCTAACA
CAGCACCTAGGATATAGCAGGTGCTAAAGCAAACACACAAACAAACAGGACTACTTATTCCCTTCCCAGGCCCCA
CCTCCCTTTCATCTTTTTCTCTTCCATTTTCCCTAGGTTCTACTCAGGCCAGAGGGTAAGAGAACATTTAGTAAG
AGGAGGAGTTTTTAACTGAAAGTGGTAGGGATCATCCTATACCTGACCAGAGTGGCAAAAAAAGAGAATGGGTAT
GTGTAGAAGCAAACAGGAAGGTAGTTCAAGTTTCCTTATCTTAAGAAACGAGTGTACCACACTACAGAAGAAAGA
AACCGTAAAGGGGAAAAATATTAGCATTTCGTCATTATTGACTGGGCACTGATAGTGCCCCTTCTTGGGCTCTGG
AAAGAGAATAAGGCTTATATTTAATCGAGTGGACTTATTATACCTGTTCCACTGAATGAGTATCTTCATCATAAG
TGAATTAGATAAAGCAAGGTGTTAAAAAAGAACATTTCCATTTGTTTGTGTCATCTCTGATTTCTTTCAGCAGTG
TTTTGTAATTCTCATAGAGATCTTTCACCTCCCTGGTTAGCTGGATTCCTAGGTATTTTATTCGTTTTGCGGCTA
TTGTGAATGGGACTGTGTTCTTGATTTTGCTCTCACTTTGGAAGGTGTTGGAGTATAGAGAATGCCAAAACTTTG
CTGAAATTTTTAACAGATTTAGGAGTTTCTGGGCAGAGACTATGAGATTTTCTAGGTACAGAATTATACTGCCTG
CAAACAGAAATAGTTTGACTTCTTCTCTTCCTATCTGAATGTCTTTCTTTCTTTCTCTTTCCTGATTGCTCTGGC
TAGGACTTCCAGAACTATGCTGAAAAGGAGTGGTGAGAGTGGACATCCTTGTCTTGTTACGGTTCTCAAGGAGAA
TACTTCCAGCTTTTGCCCATTTGGTAAGATATTGGCTATGGGTCTGTCATAGATGGTTCTTATTTTTTTGAGGCA
TGTTCCTTCAATGCCTAGTTTGTTTAGGATTGTTTGTGATTTTTCTTTTTTGAGACAGGGTCTCACTCCGTTGCC
CAGGCTGGAGTGCGGTGGCATGATCTCAGCTCGCTGCCAACCTCTGCCTCCCGGGCTCAAACAATCCTCCCACCT
CAACCTCCTGAATAGCTGGGACTATAGGTGCATGTCACCATACCAAGCTAATTTTTTTTTTTTTTTTTTTTTGTA
GAGATGGGGTTTCACCATGCTGCCTAGGCTCGTCTTGAACTCCTGGGCTTAAGAGATATGCCCGCCTTGGCCTCC
CAAAGTGCTGGGATTACAGGTGTGAGTCACTACACCTGACCTACTGAAGGTTTTTAACATGGAGGATGTTAAATT
TTATTGACAACCTTTTCTCTACCTATTGAGATAATCTTGTGGTTTTTGTTTTTAGCTCTGTTTATGTGATGAATC
ACACTTATTGATTTGTGTATGTTGAACCAACCTTGCATCCCAGGGATAAAGCCTACTTGATCAAAGTAGATCAGC
TTTTTGATGTGCTGCTGGATTTGGTTTGCTAGTATTTTGTTGAGGATTTTTGCATCTATATTCATCAAGGATATT
GGCCTGAAGTTTTCTTTTTTCTGTTGTGTCTCTGCCAGGTTTTGGTATCAGAATGATGCTGGCCTCATTAGGGAA
GAGTTCCTCCTCCTCAATTTTTTGGAACAGATTGAGTAGGAATAATACCAGCTTTTCTTTATACATCAGGTATAA
AGAGATGACACAAACACATGGAAAAACATTCCATATTCATAGATAGGAAGAATTAATATTGTTAAAATGGCCATA
CTGCCCAAAGCAATCTACAGATTCAATGCTATTCCTATAAACTACCAATGATATCCTTCACAAAACCAGAAAAAA
CTGTTTTAAAATTAATATGGACCCAAAAAGGAGCCCAAATAGCCAAAGCAATCCTAAGCAAAAAGAACAAAGCTG
GAGGCATCACATTAGCCGATTTTATACTAAAAGGCTATAGGAACCTGAACAGCAGGGTACTGTTATAAAAACAGA
CACATAGACCAATGGAGAATAGAGAGCCAAGAAATAAAGCCACACACTTACAACCATCTGATCTTCAACAAAGTT
GACAAAAAACAAGCAATGGGGAAAGGACTCCGTATTCAATAAATGGTGCTGGGATAATTGCTAGCCATAGGAATT
GGAGTGAAACTGGACCCCTTCCTTGTGCCATATACAAAAATCAACTCAAGATGGATTAAAGACATAAACGTAAAA
TCTAAAATTGTAAAAACCCTGGAAGACAACCTAGGAAATACCATTCTGGACATAGGCCCTGGCACAGATTTTATG
ATGAAGACGCCAAAAGCAACTGCAACAAAAACAAACACAAATGGGACCTAACGAGCTCCTGCATGGCAAAAGAAA
CTATCAACAGAGTAAAAGACAACCTACACAATGGGAGAAAAATATCTGCAAACCATGTATCCAACAAAGGGATAT
GCAGAATCTGGAAGGAACTTAAAACTAATTAACAAGCAAAAAACAACCCACCCCACTACAAACTGGGCAAAGGAC
ATGAAAATTTTTCAGAAGAATATATACAGACAGCTAAAAAGCATATGAAAAAATGCTTAATATCACTAATAGAGA
ATTGCAAATCAATTCTCTAGGAATGGCTATTATTAAAAAGTCAAAAAATCACAGATGTTGGCAAGGTTGTGGAGA
AAAGGGAACACTTATACACTGCTGGCGGGACTGTAAATTAGTTTAGCTATTATGAAAAGCAGTTTGAAGATTTCT
CAAATAACTGAAAATAGAACTACCATTCACCCTAGCAACCCCATTATTCTATATGTACCTAAAGGAATACAAATT
TTTCTACCATAAAGACACATGCATGTGTATGTTCATCACAGCACTATTCATGATAACAAAGACAGGGAATCAACC
TAAATGCCTATCAATGGTAGACTGGATAAAGAAAATGTGATACATATACACCATGGAATACTATGCAGCCATAAA
AACCAGATGGAGCTAGAGGTCATTATCCTAAGTGAACTAATCCAGGAACAGAAAACCAACTACCGCATGTTCTCA
CTTCTAAGTGGGAGCTAAACACTGAGTATACATGGACACAAAGAAGGAAACAGCAACACCAGGGCCTACTTGAGG
GTGGGAGAAGGGTGAGGATCAAAAAACTACCTATTGGGTACTATGCTTATTACCTGGGTAATGAAATCACCTGTA
CACCAAACCCTCATGACACGCAATTTACCTGTATAACACACCTGCACATGTACACATGAACCTAAAATAAAAGTC
AAAAAAAAAATTCCAGTGACCAATTTTTCATGTATTGGCAGTGACTGGAATTATTTTAAAAAGTTAAAATTAAAA
ATAACAAACATAATTAACATGTAAAAACATATACTTAAAAGTAAAATGACTCTGTGACAATAGACTTAACACACA
ATCTAGTGGCTGAAAATACTATTTTTTGGACAATTTTATGTTTAGAAAATTTAGAGCTGGATGCAAAATTTAAAA
ATTCAGGATATTATTTTGTCATGATCAGCAAAATGAGAATAGCTGTCTAACTGTAGTTTGTCAAATCAGCAAAAC
AAACATAGCTGGCTAAAAATGATTAAGAAATGTTAAAATACACATACATCCTCTAAAAAACAAAACAAAAATACC
AAATGGTTGCAAACCCATTCTGCAGTTCACTAAATTTCCTCAAAAAAGCTCCCAAGTAACCAATCAAAAAACGGA
AAAAATACTAAGCACTTTAGGAAAAAAGAAAATGTTTCAAGGCACAGATTAATTAGAAAACTTCTACATGTCTAC
TAAAATTTACCAATATATAAAAACTTACTGACATGTAATTTCAGTTGGACCAATTCATTTTCTAAAACATTCCCC
TACTACTTTCTCAAGAACCAATAAAAACTCTGGAGAACTAGGAAAAAATGGGTCCTATATTAAGTCATCTTTCTG
CACCTTGAGATGTATAAGGCTGGGGGTCCCTTGAACAAGATATTCCTATTCTATGCTTTTGGTTAACACTTACTG
TGTAATTACTGGTGCTCCACAGGTCTGTTACCTAAGCTCAAACTGATTCTGTAGATACATAGGTCAGGCTTATTT
GCCCCAATTTTTCTATTGCCTTACACACTTAGACCTGGGTTATTACAGTTAACACTTACTGTTACCTTCTATGTG
CCAGGCATTATTGCAAGCATTTTTTTTTTTTTTTTTTTAAATAAGACAGAGTCTCGGTGTTCCCCAGGCTGGAGT
ACACTGGCACATCATAACTCCCTGTAACCTCAAACTCCTGGGCTCAAAAGATCCTCCTGCCTCAGCTTTCGGAAT
AGCTAGGATTACAGGCACACAGAAACACATTCCAATAATTCTTTTTTATTTTTTGTAGAGATGGGGTTTCACTAT
GTTGCCCAGGCTGGTCTGGAACTCCTGGCCTCAAGTGATCCGCCTGCCTTGCACTCCCAAAGCTCTGTTCTTACA
GGCATGAGCTACTGTGCCTTTTTTCAACTGATAAAAGAGAAACACAAAAGTATACTATAATTCTTTTCCTGATTT
CAGAACATCTTTTAAGTGTTTACTTAGGGTTTTAGGATTATAGATTTATCATCATCATTTTTTTTTTCAAGTTGG
GTCCATGCTCAACAATTAATTATCATTACTTTTCCTTTTGAATTTACTGTTCAGGCTTTTTTTTTTAAAAAAAAA
TAAGAAGTGAATAACAATATAAAATGCAAAGTAAAAAAATGTTCCATTAATATATTTTAACAGTTTTGCTGTATT
TTATATTCATATGTCAAGAAAAAAAAGACTTAGAATTACTTCTTTGGTTTTGTTAATAAGTCCATTCAATAACTG
TTACATAATAGACTATACACAATGTAGGGGCTGGGGCTGGGGCTGTCTGGTTGGCCACTATTAAGCCCGGTACCT
AGAACAGTGCCTAGGAGCTCAATATATATTTATTAAACAAATAGTTAAATATTTACTTTAAGGGTTAATCTTACC
TATTTTAGGGTAAACACAATTGTATGGAAAAAGAAGAACATTTAAGAAAAAAATGCTTGATTAAATGTTTCTTCA
AGCATAATTCTGCTGGTAATATTTCTTTCTTTTTAAATGCAACTTCTGTTTTGTTTTTATTCGTAAAAGACACAA
TTTCATATTGCTTGACTACAGTACCAGCAGGCAGAGCATTACGTACAAATCAGGAGTAAAGCTTTCGTCAGTGTA
GATGATCGTATCCTGAGCCATGTCTTCTTCTGAAGTGGCTCTCCAGAAGGCTGTCAGCTCGGATCTCATGTATCT
ACGCTGATTATAAATATGTTCATGACAGGGTGGCATCTGCTTCACAGTATTGACATCCACATCTATGTGTGTGGT
GGGATATGGAGCATACATGACTTGCCGGAAAGGCAGCACAAAGCTTCCAGTTGAATCCTGTCAAAATAAAAGGAA
AATTTACTGTCTTACATGCCAAACGATATGAATAATTGTTTTTTAATTTTAAAAAATGGTCTCTGATTTAGCAAA
TCCATCTCTAAAAATTTATCCTAAGAAGCTATTCATGAGTGTGTGCAAAGTTTTAGTGACAGAAGTGTTCAGTAT
AGCATTGGTTTCAATAGCAAAACATCAGAATGACCTAAGCATCCAAAGGAAGTGGTTAAGTAAATAAAAATGAAT
TTATTATTTGGCATCTATAGATGAAGTTGTTGAAGTTTGTGTTGATATTCAAAGAGGCTTAGAATCTGCTGTGAA
AAAAGCAGATTAGAACATGCAGAGATGATCCAGTGGGGGTGGGAGTCTTAAAGAAAATACATGCAAAAGGGAAAA
AAAGACTAGAAAGAGATATACCAAATATTAACAGTGGGTATTCACTGGATTCAAAATTACAAGTGTCTTTTAATT
TCTTAATTATTCTATGCACCTCAATGAGTATGTATTTTTATAAAACAAATTTACTTCTTTCCTAATCAGTTGGGG
TAGCCAGTTACCATGTAATAATAACCTACTGCGTATGAGGCACTGTGCTATGAACAGAACAGAAGAAAGAGGCTA
TTTCCATTTTTTTACTATTATAATGCTGTGATAATCATCTTTGTATAATGTCAAAGGTATTTTCTGGAAAACTAT
ATGATTTGTATTTGCATGGAGAACTATTAAATATTTTTTCATTAGATCATAGTTTTGAAATTTCCTCTAGGCATC
TGGTATAGGTCCCTTTCAGAACTAAAATTATATAATTAAATGAGAGGGTGCTGAGGAAACAAAAGTATTAAGCAC
TTTATTATGCCTTTAACAGGAGAAGTTCTAATTTACAGATATGTATAACTCAAATAGAAACTACGTTTCCTTTAA
CTAGTTTCTATATTTAAATACTATCATTCTGAGTTCATAGCAGCACATTATTGAAAATGCACAAAAGCCTAATAT
AACTTGTATTTGCAACAATTTTTAAATTTTTTTATTTCATTTTGCCGTGGCTACATCCTCAAGAAAGTAGTCACT
ATGAGACAATCACATAACCATTAGGAAAGCTATTTCTTCACGTCTTGGGACTATAATTGAAATAACAATAATAGA
TTCAACAAGGTGAACAACTTTCTTCCCTTTAAGTATTATAAATAATTGCTAATTTACATTTCTCTGTTTTGCTCC
TTGACAGTAGACTCAAATAAAAGAAAAATACAATTTTTTTCTAATATATATTATATATAGAAATATATATATTTT
AGTACACATACATAGGAATTGCTTAAATCCTATAAGCTTCTTAAAGATGTTTAAAATTTTTTTTCAATAAAATTC
AAATCTATTTAGTTAAAAAGAAATGATCTTATTTTTGATGTGCAACCTGATTTAAGCATGTGTTAAAAAAAAAAA
AGTCCTGAGGCTAGACATGTAGGAACAGGGACCCACCTGGAACACAAAGGGTATTCTATGGTGTTTCACTGATGA
TACTAACTATAAATCCATAAGACATATAGTCTATGTGCAGAACTGTGTAAAGGAAGTCAGTCTCTGGGCATGTCA
ATATGAGATACATTAAATGCTAATATTTAAGATTTGTCTATAAAGAGTCTCAAAAATGATTTTAGAAAAGTGGTT
TCACTTGTGATAACTAGAAACTATACCTTTAGCAGGCCTTGTACAAAGAGCCCTGACTCATATTTAAATGATGAT
TCTGCTTCACATAACCTGGAGCATTTTCTCTCTGCTGGAGTCAGAAAAAGGCATAATGTTCTGACTATCTATAAA
AGAAAATATTTTAGCATTAAAACATGAAGTAAAAAGACCACTGATTTGCTTATGAAAGATATCTGAAATTTTAAT
TGTTATTATCAATAAAACATATCCTAAGAAATAAGTATTCTTTAGTCACCTGGAATCCATGGGATGTAACAGTTG
TTGCTTAAAATAGGGATTCTGTGGTCAAATAAATTTAGAAAATGCTTGGTAAATTTACTGTATGGCATCTCAAAA
CCTTTAACATTTGGCTACATACTGTGACTCTTCAGGTGAATTATATCATCTGCAGACTTTCTCAGACTTATTTGA
CCATGCAACTTTTATAGCTTCTGTTTAGTGGGTCTACACATGAAATTCGTTGTAAGAAATATCAAAGAATGTCCA
GATCCTCCAAAAAGAAGAGAATTAATTATTGAATTTGATTTAAAATAAGCAGGTCATTGGGTGGGATTTAGAAAT
CTGATTCTAATAATATTTGACCTACAGCTCTATTAGGAAAAATAAAAAGGCATGTAACTATCTTGAAATTCAAAC
CATATCCACAGTTATTATGTTAAAGGAGTGGTTTTCAACCTAGAGTGGTGGCAACTTCTTTCTCAAATTAAAACA
AGATAAAAGGTAAGCTATTCTCCCTCAAGAGGATGGAGGATGGGAAAAATGTTTAAAAAAAAAAAAAAAAACACT
CCAACTATGGAGCCTTTCTCCCTTCATAAAGCAGCTCGGCAGTCACTCTGTGCAACCTAAGGCTTTGGAGATCAC
AGATGGAAAGCCACCTGTTTGAGGTAACAGAAGGAATAAGGTCACTAGTTCGTAGATGCAATATAATGACACAGG
TATACTAAGCTCTCATAAATGGTTATATGAGAAATATAAATTAAGGCTCATGTAAATATACAAAGTAGCTGATTA
CAAAAAAAAATTATGAATATCTTTGTAAAGTATCATTTCCAACATATTTCCTATGTAAAACTTTTTTTAAAAAAT
TAGGTTTGCTGAAATTGAAAGATACACATACTTATCTCTGAACTCTTTCTAACTAATGGTCAGTGAAGAAAAGTG
CAAAATCCTTTAGTTTATTAGCTAATGCTTGGAAATGTAACTGTTCATTAATCCTTAATTAACTCAAGTAGCACT
GAAGGAAAGGGTCAGAAACATTACTGAATAAAGTATAATAATCAATGACCACTTAATCCCAATAGCTCCCTAGAA
GGGACAGATTTAGAAGGAAAGCGAAGACAATGAAATCAAGATGAATAAACAAATAACATTTCTTTGGAACTACTA
CCAAAAGTACATGACTATCTTCAGATTTGTTAAAGATAACATTGGGAAATAGAAGAGTAATTTTTTTTATATATC
TGATTTTAATATATTCTCAAAACCATTTATACACTACTGACACTGGTATTTCCGAGCTATCAAAATAAACTGATA
AATGATTCTTACTCAGTTTATTTCAAACTCACTGTTGCCACAAGGTGTCTTAGCAATTTGATGAGATTACATTGC
CTCCTTATACTACTAGATCATTTTAATTGCAACCTACCATTTAAATGACAATCCATGATATATCATCAGTCTTAA
AGAGTCAAATCATTTGCTAGATTATAAAATAAACTACCTTATTTACTTTCTCTGCACTGCTACCTACTACAACGG
AACAGCCACAGGTTTGCAAGTGTGAGCTGATGGCACTGTAAGTTAAAGAAAACAGATTAAAAACATTGCCTATAA
AACAATTTAACAAACTAAAAACAAAAAAAAGTAGGTGAGCTCTTCAAATAACTCAGAATAGCTTTATATGATAAA
CACCGAAGCTATAAGCACAATGTTATCTTTTATTTGTATAGGAACCTACATTTTCTAGAGACCTTTCACAGAAAT
TTTCTTATTGAGCCTTAAAACAGCCCAATTAGTCAGTATAATATCATTTAATTAATGTATTTATTTATTGAAATA
CCATCATTTTATAGCTGAAGAAATTGACATGTAGAGAGATTAAGTGACTTACTTAAAGTCAAATGGGATTTAAAA
TGATGTATGAAAGGCTGACACTGAACAGATACAGGACTAAAGTGCTTCTGATTCAAGCCATTAAGGCTCTTAGGT
TAAACACACTCATGCCTCTGATACTCCATCATGAGCCTAAAGGAAAAGACTGTGAACATAAAAGTGAATACTTTA
TACTTTTACTTCTCTTTTATTAAAAGTAAAATTTCATGAAAATCTGTAACTGTGAAGAAACTTTAAAACAGAATA
TAAGATAATACATGTAAAGCAACTAGTAAAGGAACTAACATGTAGGCACTCAACAAATACTGGCTATTTCTAGAA
GAAATGTAAATAGGAAATGTTAGCTATGAGCTATTATTAAGTGTTTTTATGTTCCAGGCACTGTTCTAAGTGCTT
TATATTATTTATCTTACTCAATGCTTATAACAACCCTACACATTAGGTACTATTACTATTATTGCCATTTTACAG
ATGAGGAAATAGGTGTATAGAGAATTCAGGCACCTTGCCCACGGGTACACAGCATTAATCCAGGGAGTCTGGTTT
AAGGGCACAAACTCTTAAGTACTAAACTCCACTGCTGGATGGAAAAAGATCAGTATAAATATGAATAATTTTGTT
CTACGCCTAAATAACTTAAGTTCATCTACAGTACAACTTAATATGAAAGGATTCTGTTAGCTTTAATGAGAAGTA
AAACAAGAAACCAGAATCAAGCAAGGGGCCATGATTTCTTGTCTGGGATGGAAACTCGGTTTCTTTAAATAGCAA
ATGGAATAACACCAAATATATATAGAAATATAATGAGTGAAAAATAACACAAATTTAAGCAACAGTTCAAATACG
TAATGTCCCTAGAACAATCTAAGTAGACAGTCTGTTATTTTCTTTCTTCCAAATCTTGTCATAGGTGAGCATAAG
ATGGTATCTGCTTCATCCAGCTTTTATGAAAAGAAAAATTCTTACTTGAGAAGAAAGCCTTCATGACAGCTGTCA
CCAATATCATCATCATTGAGTACTGTATCAGCTATCTAAAATGCATCAAAAAATAAAAAAATTAGTCTGGCTGTA
ACATAGTGTTGAAATAACACTTTTAATATACAAGTTTTCGGAAGTCTGGATTCAATATAACACACTGCCTTCATT
TCCGAGAATCAAGACTCCCCAAAAAACAATCTCTGTGCACTACCATAAACTTCAGAAGAACAAATGTGAAAGCTG
GTCAAGCAGGTTAAACAATTTTTACAAGAACAACTTCCTCTTCTGAGCTGTCAGAATCAGGAGACTAACCTAAAT
GACAAAATCAGAAAACAACAAGAATAGTTTCCTAAAGGTATCTCTTAACACTCATAGTGTGTGATTCAAAACGTC
CTCAACAAATGATTAAGGAAACTAAATTTGTGACTACAAGTAAACTTCCATTAATGGTTACTACTTTGGCACACA
GTTTTGTTTCAAAAGACACTACATTAAATATTAATTGCTCCTATAAGAGCTGGGATCCTCCCACTTTTAGGAATT
ATAAAAGTAATGAAATAAACAAAATGAATTTAATTTTGTCATCACTGATCAAAAATGCCTCTGTTTTGCCATAAA
ATCCAGGATTTTGTGTGTGCTTATTTGCTAAAGTGGCTAATACTGTATGTGAATAGTATGTATGACAAAGTCCTT
ACTATTAAAATTAGAATATTAATAATATACATAATAATACTATAACCCCAAAAAACTCATAAAGTGTATAATTGC
TCTCATTTAAACTTACATCTATTTCTTCAGGAACACTGTGTGATTTCATAGATGAAAGCAGTTCCATTACAGGAA
TCACTTCTCCAGTAAGCATTGGAATAATACTCTGACCCTGCACAATAAAGTGACATGAAGTGAAGAAAATCACGT
AATATGAGAGAAGCTGGGCAATAAAAAATAAAAATAACATCAAACAATAACATTCTTTGATGAAAATACTTCGTA
ATTTGTTCAAACACAGTATCAAACAAGTCTACTACATGTCTAAAGGATTTATATGCAATCCAAAGCTCACTTTTA
TTCTTTCTTTTCTTTTTTTTTTTTTGAGATGGAGTTTTGCTTTCATTGCTCAGGCTGGAGTGCAATGGCGTGATC
TCAGCTCACTGCAACCTCTCCTCCCAGGTTCAAGTGACTCTCCTGTCTCATCCTCCCAAGTAGCTGGGACTATAG
GTGCCGCCACCATGCCCGGCTGATTTTTGTATTTTTAGTAGAGACGGGGTTTCGCCATGTTGGCCAGGTTGGTGT
CAAATTTCTGACCATGCCCGGCCCTAAAGCTCACTTTTATTCTTTAGAGAGTATGGAATCATTGGTTTATCGTTT
ACTGTTACATGCAATGATTAAGTCATCATGCCTCTTTTAGAAAAGATCTCCTTTAAAATTTGAGATAAAAAAAAT
TTGTTAAAGGTCATCAATATATTTCATATTTAAAAATGAGGAAAACCAAGCACAAAAAGACTTTGAAATCCTTAC
CAAATAGGTAAGGAAAACTTGAATCAATACCTAACCTCCATACTCATAAAAGTATAATCTACCCAAATGCAAATC
AAAATCAGCACATATATTTTAAGAATCAATAAAACAGAAAAATTCCCTTTAGAGCTATTTCAAGATATTACTACT
TATTACATCTTGAAATTGTAATTTTGAAATTTGTAGTCTATAGAATCAAACTGAAAATTCAGTATAACACATCAC
AAATGTAAAGTGTCTCAAATATGGATGGTCCCTCATTTATTCACTACCACCACCAGTCTCATCAGTTTTGTGACC
AACTTGGTTAAGTAAATTTTTTGAGATATAAATGAAATTGTCAAATGACTATGCATTTTTAGCTAAACATATTTT
TTAAATCATACATTATTTAAGATGAATTTAATACTAGTTGTTTTTCCTCACTTATTTTATGAAATGATTTTACTC
AAAGTCTTCATAAGCATCTTTAAGTTAGAATCTTTGTCAGACCCAGGGCCATTTTTGGAGTAACCTTAACCAGTT
TTTCAGAGCCCCATATTATTAAGTTGCTTGAGAATTTAAATGTGATGCTACTTCTGGAAGTTTTATCCTAAGCCA
TATGCCCATTTGCATAATGCTGAAAGTTTTATTTAAAAAAAAACCATCCTTTAGTAACCTCCACAACTAACTATT
CACTGTTTTTAGTTTTTAAAGTAATAATTATCATGCCTGTTTACAATTACAATTCACACATTCAATCTAACAAGA
ATAATGACTAGATCCGTGTTAAATTTCCTTCCCTGTGAAGCAATTTTATCAGATGACAGCTACAACTGAAGTTGT
TTCAAACTAATGCATCATCCCCAAACAGTATTGTTCAAAATAAAGTCGTTGTGAGATTTGCAAGAACTCAATCAA
AAGGCAACTCCTCCTTTTCGGGAAGAATAATTTTGGGAAAATATTTCCTCTTAGGTTTAAGCATACATAGTATTT
CATTCACAGTATCTCAGACATTATCAGTATAAGTGAATGAATAGCCTCACTGAAGCTCAACAACACCAAAAAAAA
AAAAAAAAAAAAAAATCCTACAGGGCTAAATACAGAAGAGGCTCTAAAAGAAAATCTCTTAAGTTTCTATTCCTC
CTTGTACTTCCCAAACTTGAACTTCTCAGCAGTAAGATAACATTTTTAAGAAGAGCACTTAAAAGAGAGACCAAA
ATTCATTAATAGTAGTCAACTTAAGTAAAGGTTTCTGGTTTGAAAAAACAAAATCCCAGTAAAAGCAGAATTTTA
GTTGGTTCTAAGTTTCCTCAACTTGCGATAAGTTTACTTAATTAGTCTACTAATAACTAGTGGGTTAGAGGGTGC
TGAAAGTTACCCCATTCCTGGGGACCCTGCTTATTGACCAGCAAATAAGGACTGGGATTCTTTGGGTAAAGGGAA
ATCTTTTCTTGTTAAGTCAGACCTTTACACAGAATAACTGTCTCTGAATTGGAAAGCTATCTACAAAAGTACAAA
CATAACAATTTGGTAAAGGAGATCATTGTATTGGGTTCTGTATTATGGCCATGTATTTTCACAAGTTTTTTTTTT
TAATTACTTTTTTAAAGTATCATCTGTCTCATTCATGCTAAAAAGAAGCAAAGAAAGGCAAAACAGCCATGTTTA
AAATATTGGAGTTTTACAAGGAGCATTGAGGGTCACCCACAAGAGGAAATGGAAGTAAAAGTGAAGAACTCTTTC
TTCACTGGAGATTCTCCTTCAAAAGAACTTCTCTGCTTTACAGTGAAATAGTCTGTACTTAGTTTCCGCAGGGGA
AGCCACACCCTTGTAACCATGCTTCTCAAACTCTTAGTGTCTGTTCCTGAGGGGCATTCAAAGCCAAGGGATAAA
CATGGCACATTTTCCTAGAGGAGAGGGTAAGAAATATCACTGACAAATTTTAATACTAAAATAGTTATGGAATAA
AATGTAAATTGCATGAGTCTTAACGATACAACATAAGACTTAGAAGAAATATTGTGTGGACCTGGGCCTACACCC
CAGACAGATACCTCAGGGGTACATATGCTCTCCTTCTGTTACAGCTACTTCTAGGGAAAGGTTCGAGAAGTAGTA
CCTTAAAGAACATATCAGAGACAATTTTTTTTATTTTTACTATGAACAAGTTATCCAAAATTTATTCTGGGCAAA
CAGAAAAAAAAAGGGAGCAAATATTAATTTGTAGATGCAATTACTATTTTCCTTTGTTTACTGATTTAACTCTTT
GGGTTTAAGATATGGAAATCTTCCTCCAGTTTATTCTGTACACCTCCATAAAAGCTCCATTAAAGGCTTATTCGT
ATGTCTCCAAGGCCTTGACAAATGTAGCCATCAACCTTATACAGATACATGCTGTGAGAAAAACATTTGACAGTA
TGCAATTTGCATATACCTGATCTTCCATTCTCTCTGTGCCTTCTAAGATAATCTTCTGGACATTTTCTTGTCTTT
CCTGAGCAAGAGAAAATTTATTTAAAAAAACAACCCACAACATTTTGATACTTGCTTATTTTTCAATAGACATGT
TCTTGTGTAGTAATTTAGTTCACAAGAAAAATACTTTCTACTTTAGGGAAAAAATGGGGGCAGGGGTAGGAAATT
AACCCAACAAATGCATGTTCTCATAAACAATACAAAATAAAATCAAAACAACCTTTATTCTGCAGTGAAAAAAAG
ATAACTTCACAGAAAACAGTCAATGTAACATCTGCATAGTTTCAAAAAGGAAAAGAATGACTTGCACTTTTCAAA
TTAAACATTATGATGTTGTTTAAAAGATTCTCCTGATTTTAAGAGTTTCATAATGTGAGAAAAAAGGAAGTAAGC
CTGCAAACATAGTAAAAAATTATTCTTTTAAAAGATATTATTTTTCCTTACTATTGGGCAAAAGCCTTTTAAAAC
TGGTAATGCTTAATGGACTTTCAGGTTAGTATCAAACTGGAACACAGGAAGGAGAATTCAATGTGTTCTTTAGAT
ACATCAAAACTATACTGAAATGTAAATAGCATTATATATTCAACTACAGGATTTAGGAAAACAATAATTTCTGTA
AGATTAAAAGGAATTCTCTTGGGAACCATTCCATTCAACCTCCTCATTTTATGAACCTGGGAACTTGGCAAAGAG
GTTAAAGAGACCAAAGGCTACATGACCAACAGCTTATGAAACTATTACTTTGAACTGTTATACTTACACATAGTA
GTAAGCAAAAGACAGAATTGTGCAATGAAAGGGAAACAAAAGGTATTAGAGTCAAAGGCTCCCAAGAAGAATCCA
GGGTCTAAAAGTTTCTTTATTTGTCTAAGCTTTAGCTTTTCATCTATAATGTGGAGCTACCATTTCGTACCTTCC
ACAGTTAATATGAAGATGACAGGTATCAGACCAGATGTATTTGTATCTAATAGGGTAAATGCAAAATAAATAACA
TTTATTGTTTGATGTTCACTGCATATAATTAAAAAAATAAGATTTATATGTACCAGAAAATAAGCTTTCAACAGA
TAGGTTAACATGATTAATAAGCTGAAAAATCACTTACCTTATGCATCCATATTCTTCCTTTCCGGATTATATGTG
TTAATCTATCAACACACACTCTATGAAGTGGGAGGTAGAAACTAAGTTCTGTCTGTGGAAGTATAATTGATAGTC
CATATGTGCTGCGATCCCCATTCCAGTTTCCATCAAAGATTAATGAAACAATAATCACTCCCTTTTCAGACAAGA
CAAAAAACTTTACATCTATAGCACCACTCTCTGCATTTCGAAGGATTTCTCCATTTAGAGTGTGGTTGGCAAGAA
AAGTTATTTCTCCATCACTGAGAAGTACCTGTTCTGTCTTTGGAGCCCAAATGTGCCTTACTCTAGGACCAAGAA
TATTGTCCCAGTAAGCAAAAGTAGCTGCTAATAAAGGTGATTTGCCACTTAAAGCAATCTCTGTCTTGGCAACAG
CTGGAGATGGCGGTGGGCAAAGAGTCGACATCACTGCATTCCAACTGTCACATTATCCAAATGCTCCGGAGATAT
CTAAACAATGACATATGAAACCAATGATTAGGTTCAGCAATTTAAAGATATCCATCAAAACCCCAAATGATTTAG
ACATATTTGGTTTGTCCTCTTAAGTCAAAGATGTGGAATCCTGTTATCTCCTATCAGGATAAAGACATTCAACTA
GCACAGTAGGTGCACATTAAATGTTTGTTGATATGATCATTTTACAAGACATGGTAACTTGTTACTTATATTCAG
GGCATACATTTAGAAATTCAAAGAAATAACTTAAAAAAGGGCTTCTTTACACTGATATTAAATGTTACATACTAA
AGCTCATAGAATAGACCCGCAGTATTCCCAAATATCCAGTCCATGTGCAATTCTAGTATGACTGGAGATTTGGCC
CCTAACCCATAGCAACTAAAAAGGAGAAAAACAGGAAGGGAAAGGCTCAGCTAGAGACTGACACTTGTGGGTTGA
ATTGTGTCCCCCAAAAAGATATGTTCAATTCCTAACCCTTGGTGTACGTGAATGTGACCTTATCTAGAAATAAGT
GTAATCATGTTAAAATGGGGTCATACTGGATTAGAGTGGGGCCTAATCCAATAACTGCTGTGTTTATAAGGAGAG
AGATTTGGAGACACAGAGACAAATGGTAGACAGCCATGTGAAGACAAAAGGCAGATACTGGATTGTTGAAACTAC
AAGGCAAGAAAGGAACACTCAGGATTGCTGGTAACCACCAGAAGCCAGGAAGAGGCAAGGAAAGAGTCTTCTCTC
TTGAAGATCATGCCCCTGTCAACACTTTGATTTCGGACTTCTAGCTTCTAGAATTGTGAGAGAATAAATTGCTGT
TGTTTAAAGCCACTCAGTTTGTGGTGCTTTGTTAAGTAATCTTAGAAAAGTAATACAACACCTAACAACAGAAAT
ACTTTAAAGCCGCTAAAAGGTCAAAAAAAAAAAAAAAAAAAAAGACATGGAAATACCACAAGTCTGGAGCCATAA
CAAAAAATGGGCAAACAGTCCTGTATCCTCAGTGAACTCTCTGGTTATGAGAATACTGAAGCCCGATCCTGATGT
TTAAAACGACATTGAAGTATCAAGACAAAGATAAAAATATTTAATATGCTAGCCAAGAAACCAATACAGCATTTC
ATCACTGCAAAGAGAGTTCTACACTAAATGGCTAGAATTTAAAAGCTTTAGTTATTTAGAACACGTAGAAAACAG
AAGGGCTAAATAGGGCCCGTTCAAGCCTTTGAATTTAATGAGAAAACAGACATGAGGAGAAGAACATAAACGCTC
ACATCCAAGACAGAACCCAGGGCTCTTGGTCCCCTTGCTCAACTTGTACATCTTAATCCACATAAACATACCACT
CTAAAAAGGTACATCCTATGTGATATTAATGTAAAACAAATCATTCTTGCAAATACAGTTATGTGCCATGTAACG
TTTCAGTCAATGGTAGACTGCATATATGATGGTAGTCCCATTAGATTACAATGGACCTGAAAATATGCTATTGCC
TTAGTGACACTGTAACCATCATAAGGTCTTAGTACTATTTTGCAAGTTATTTAAAGTATAGCACATACAATTATT
ACAGTGTACAACACTTGATAATAAACTACTACATTGCTGGTTTATGTATTCACTATACTATGCCTTTTATTGTTA
TTTTAGAGTGCACTCCTTCTACTTTTTTTTTTTTTAAGTTAAATGTAAAACAGCCTCAGGCAAGTCCTTCAGGAG
GTATTCAACAGAAAGCACTGTTATCATAGGTGACAGCTACATGTGTGTTATTGCCCCTAAAAACCTTCCAGTGGG
ACAAGATGTGGAGGTGGAAGGCAGTGAGGTGGAAGGGAGTGATACTGATGATCCTAATCCTGTCTATGCCTAGGT
GAAAGTGTGTGTGTTTTAGTTTTTAACAAAAACGACTAACAAGTAAAAAAAAAAATTTAAAATAGAAAATAGAAA
AAAGCTTCTAGAATAAGGATACAAAGAAAAAATATTTTTGTATAGCTATACAATGTATTTGTGTTTCAAGCTAAG
TATTTTAAAAGTTAAAAAATTAAAAAGTTTACAAAGTTAAAAAGTTATAATTTTTTATTGAAGAAAAACTGTTAA
GATAAATTTGGTGTAGCTTCAGCGTACTGTGTTTATAGTCTACAGTGGTGTACAGTGTTCTAGGCCTTCACATTA
ATTCACCACTCACTCACTGACTCACCCAGAGCAACTTCTAGTCCTGCAAGCTCCATTCGTGGTAAGTGGCCTACA
CAGGTATACCATTATCTTTTATACCATACTTTTACTGTACCTTTTCTCTGTTTGCATATATTTAGATAAATATTT
ACCACTGTGTTACAACTGTCTATAGTATTCAGTACAGTAACAGTTGTACAGGTTTGTGGCCTAGGAGCAACAGAC
TATACCATACGGCCTAGGTACATAAAGGCTATACTATCTAGGTTTGTGTAAGTACACTCTATGATGTTTGCATAA
TGACAAAATCGCCTAATGATGCATTCCTAAGCAATGTGTGATTGTACTATAATTGAAGACTTGTTATCTAAGACT
GAAAGTAAAAAGAATTGCAATTTCACCTAAGCAAGTCTAAAACTGTGAAGTCTATTTATAATAATAGCAATACAA
AGCAGCTAATAGGCAAACTATGATATACCTATCTTTGCCATATGATTGCTTTGGGAGCTAACATTTGATCTGTAA
ATGTATGACAAAGTAAACAATTTTACTTAAAGAATTTCATCCACATCTTGTCAAGAGAGTTCAGTCTGATGGAAA
GCACTGACTTCTATTTACAGAGCATTAGATGAGTGCTTTTATCATATTATGAGTAGGCATACAGAGCCTGGCAAA
ACAGTTAACTCTAAGTATGTACAGAAATGGTTGAACACAACGACAGTTTTAACACGTGTATTTGTAATTTCAAAA
ATTCATTTAGGTAATATTTACTTTTAAATATGTTGTATCAATTTAATAGTCTTAAGAGACAGCACTAGATATAAG
CCGTACAGCTTCTTTAAAATATCCACTGTTTTTAATACAATGTAAGCAGTCAGTTTACAATGATCAAATATAGGA
ATGTAATCTGAATTGAAATGGTAATGACACTACTGCTGTCATAACTAACAACAGCAAACTGGAGGCCAACATAAT
GAATTAAGTTAACATACAACCATAAAATTATATTGCAAACATATTTTTCTTTCATTCTTTTAGGTTAAAAAGGTG
GATAATCATAAAGGCAATATTACAACTCTAATATTTCATCATTAAACTGAAAATAAAAGTATTTCCTAAAACAGA
ACTGAACCCTGGAGCAAAATCTGATTGAATTATAGGGAAACTTTTACCACGTTGTGAAAATTGAACTATTATACT
GCTAGTTACACTCTCACTCCTAACAGAATAAGAAAAAAAAAATGGGCCGGGCATGGTGGGTCACACCTGTTATCC
CAGCTCTTTGGTAGGCCGAGGCAGGTGGATCACCTGAGGTCAGGAGCTCAAGACCAGCCTGGCCAACATGGTGAA
ACCCCACCTCTACTAAAAATACAAAAAATTAGCCGGGTGTGGTGGTGGACACCTGTAATCCCAGCTACTCGGGAG
GCTGAGGCAGGAGAATCTCTTGAACCCGGAGGTGGCAGAGGTTGCAATGAGCTGAGATGGCGCCACTGCACTCCA
GCCTGGGCGACAGAGAGAGACTCTGCCTCAAGAAAAAAACAAACAAACAAACAAACAAAAAGAATAAGAAAGAAA
ATGAAGGACAAAGATCATACTGAATTGCTTAGTTTTAAATCCTACCAAAAGAAATAGCCTGGGAAATGAAATGTC
ACAGAGAAGTATAATCAGGAGAGCTGTACAATTATTTTACTAATACTTGAAGTCATCGTCTTTGGTGAGAAAAAT
CCATACATGCAAATGCAGCTGAAAAAAATCAGCTCAAAACCAATAGTTGTTTATGTACCTATCTTACGTACATGT
AGTGCTGTCTACTCCAGAGAGTTACCAAACATTAGCCAGTCTTTTGAGGGAAGCCAAGATTCAAATTGAGTGAGA
CGGTGGCTTGCTCACAGGGTTCATGAGAGGTTTCCCAATACACTTTCTGGAAATAATCCCATACATGCAGACATG
ATTACATTAATTAACATCTGCTAAAACTGTTAGTAGAGTGCTAAGTTTGAGGTTTTGCTTTTTCTTTAAACGTCT
GTTAAAAAATCAACCATCTCTTCCCTGATTGGTATTTAGAAAGGTGGTTGGTCCACTGCTATTGTAGTGAAAATT
CTACAATCATAAAGCCCTCACTTCTTGTTTTTTAGAGACAGGGTCTCGTTTTGTCATCCAGGCTGGAATGCACTG
GCAGGATCATAGCTCTCGGTAACTTCAAACTCTTGGGCTCAAATGACCCTCCTGCCTCAGCCTCCCAAGTAGCTA
GGACTACAGGTGCACATCACCACGCCCGGCTAAGTTTTTAATTTTTTGTAGAGACAGGGTCTACGTTGCCCAGGT
TGAGCTTGAACTCCTGGCTTCAAGTGATCCTCTTGCCTCCGCCTCCCAAAGCTCTGGCATTACAGGTGTAAGCCA
CCTTCTCCAACCTGGCTCTCAATACTTGTAACCATGCTGTTTATTTTCTCCCAGCCCAAAGAGAAGCAGGATCCT
AAACCGTCCACTTTCCACAACAGGAGCTGCCCAGGACCACTTCAAGGACAGTGAACTGTTTACAGTACCAGAAAG
TTCACAACACTTTCTCAATCTTCAACATCAGGGAAGACTGGAAGGTGAAGTTCATATCACTATCTGGCCATTTCT
CACAGTTCCAAGTTTCTCAGACAATAGGTAGGCTAACCTAGTCCTCCTGGGAACTATCTAATTAACGTAGAATAG
AACCCGAGGGCAGACTTGAAAAACAGAAGTCCTCCTTGGTTTACTTTGTTTCTCTGAAAGCAAATTGTGGAGTGC
CAACATAGCCAAACAAAATATTTTATCAACTTCATAAGGTGCTTGTAATTTTTTCCTGGAGCAGGTAAATGCTGG
CTTAGTGAACAATCTGGAATGTGGTAATTACTCTCGTTCTTGTTTCAGATGTACTATCAGCATGTAGCAGTTTCC
AACTGATTCAGGGTTTTCCTAAAGTGGCAGGCCTTGGCAGAGGTGGTGACAACAATGCCCGTGTCAAATGACACC
GTATTTCAAGTATTCTGACTCCAGGTTATTAATATCCCCTATATGATAGTCTTGTTTCTGTGATATTCACAGATT
ATGTTAAAAGTTTCCCAAAGTCTGAGAAAAATCATATCTTAACAGTATCTTTTTTTTTTTTGATCCTTTGTACAA
AAGTAGAAGTAATGCCAGACAGATTACGTACCCTTGTTGTGAACAACTGGTGCATGGCAACTGTTTGAATAGAAA
TTTACCAACTGCCACAACCAGGCAACTACTCTCCCAGAGCCTAACAATCTCGATTGCATCTGAAAGGGCCACCCC
TCCTGGGAAAGTGCAGGACCTCCCTCCTGTTTCTGAATACAAAGCCTGGTGGTGTTCAACGCGGCCAGATAGACC
CAATGAGCACACGGACATGTAATCTGTGCACTTCTTTAGACAACTGATTACCATCAGTCAAGTGATGCCCAAGTC
ACAATAGTCACTTCCTTTAAGCAAGTCTGTGTCATCTCGGAGCTGTGAAGCAACCAGGTCATGTCCCACAGAATG
GGGAGCACACCGACTTGCATTGCTGCCCTCATATGCAAGTCATCACCACTCTCTAGAAGCTTGGGCTGAAATTGT
GCAGGCGTCTCCACACCCCCATCTCATCCCGCATGATCTCCTCGCCGGCAGGGACCGTCTCGGGTTCCTAGCGAA
CCCCGACTTGGTCCGCAGAAGCCGCGCGCCGCCCACCCTCCGGCCTTCCCCCAGGCGAGGCCTCTCAGTACCCGA
GGCTCCCTTTTCTCGAGCCCGCAGCGGCAGCGCTCCCAGCGGGTCCCCGGGAAGGAGACAGCTCGGGTACTGAGG
GCGGGAAAGCAAGGAAGAGGCCAGATCCCCATCCCTTGTCCCTGCGCCGCCGCCGCCGCCGCCGCCGCCGGGAAG
CCCGGGGCCCGGATGCAGGCAATTCCACCAGTCGCTAGAGGCGAAAGCCCGACACCCAGCTTCGGTCAGAGAAAT
GAGAGGGAAAGTAAAAATGCGTCGAGCTCTGAGGAGAGCCCCCGCTTCTACCCGCGCCTCTTCCCGGCAGCCGAA
CCCCAAACAGCCACCCGCCAGGATGCCGCCTCCTCACTCACCCACTCGCCACCGCCTGCGCCTCCGCCGCCGCGG
GCGCAGGCACCGCAACCGCAGCCCCGCCCCGGGCCCGCCCCCGGGCCCGCCCCGACCACGCCCCGGCCCCGGCCC
CGGCCCCTAGCGCGCGACTCCTGAGTTCCAGAGCTTGCTACAGGCTGCGGTTGTTTCCCTCCTTGTTTTCTTCTG
GTTAATCTTTATCAGGTCTTTTCTTGTTCACCCTCAGCGAGTACTGTGAGAGCAAGTAGTGGGGAGAGAGGGTGG
GAAAAACAAAAACACACACCTCCTAAACCCACACCTGCTCTTGCTAGACCCCGCCCCCAAAAGAGAAGCAACCGG
GCAGCAGGGACGGCTGACACACCAAGCGTCATCTTTTACGTGGGCGGAACTTGTCGCTGTTTGACGCACCTCTCT
TTCCTAGCGGGACACCGTAGGTTACGTCTGTCTGTTTTCTATGTGCGATGACGTTTTCTCACGAGGCTAGCGAAA
TGGGGCGGGGCAACTTGTCCTGTTCTTTTATCTTAAGACCCGCTCTGGAGGAGCGTTGGCGCAATAGCGTGTGCG
AACCTTAATAGGGGAGGCTGCTGGATCTGGAGAAAGTGAAGACGATTTCGTGGTTTTGAATGGTTTTGTTTGTGC
TTGGTAGGCAGTGGGCGCTCAACACATAATTGGTGGATGAAATTTTGTTTTTACCGTAAGACACTGTTAAGTGCA
TTCAAAACTCCACTGCAAACCCTGGTAGGGGACAGCTCCGGCACTGCGGGCGGGAATCCCACGGTCCCCTGCAAA
GTCATCGCAATTTTGCCTTTACATGTAAGAATTCTCTCAAGCATGATTTTCACACTGGGGAATGTCATTTTTGCT
AGTTGCAATATGTGGATGAGTTGTTTTTTTTTAACTTTTGAAAAACGTACCATTCTGTTTGATGTGTAAAAAACA
CAAAGATTTTTGAAACCTTGCGTCTTTTGGTCTGCAGGTGTATAGATTCCACTTACTACAGATGAGTAGCATTTA
CACCACTCAGATGTGTAAAAAAACAAAGGTTTTTTAAACTGTGTGCCTTTTGATCTGCAAGTGTGAGATGGCACT
TACTACAGTGAGTAGCATTTAATCTTTTTCATCACTAAAAATCACACAGAACGTTTTAATCATTCACCGAGGAAG
AAAGGGAGGAATAAATACACAAAATGGCTCTCAACGTCTACACCTTCTGCAGAAACAGACCCTTTTCCTACTGTT
CTATGCTTTGTGAAAGTTGATCATACAAATTGGGTCATTCTTTTTATACCCAACTAAAATAGTGGGGGTAGGGGG
TAGAAAAGCACTTAGGACAAATGACACTGCTCCCACAGTGTAATTCTCTCCAAGTCCAGCTGCTGCAACTGCCCG
TTGTGACCTGAGACCAGTTTTATCTAATAGTTGCTAAAATGACCTGCTGCAGCTCTAATTTTATCTACCACCATC
ACTCACCAGTTGAAACTCACCAGCTCCTCAGATCCTTAATAGTGCCAATGAATTTTCTCAAAGAGCACTATGTAA
CATTTCTCTTTTTTAACAAAACCTCCCCCTTTTCTTTGTTGTGTGGATATACCGAAGACCATCTGATCTACATGT
ATGCCCTAATTGCAATTCTTTCTTCCCAAATAAATCACTTAATTTAGAGATTCATCTCTGTATTTTTATTTTGAC
TGACAGCTTATAACAAGTAGCTAGCATTTACCAAGTTTCTACACTGAGTTGTACTTCACTTATACGTGGAATTAA
AAAACAACTGAATTTATAGAAACAGAGTAGACCCTTGGTTGGGGGGCTTGGGGGGAAAGAAAATTGTAGGGTAGG
GTACAAAGTTGCAGTTACGTCTAATACATCTAGAGATTTAATGTACAACATGAGGACTAGCGTTAATAATTGTGT
TAGTCCATTCTTACACTGCTATAAAGAAATAACTGAAACTGGGTAATTTATAAAGAAAAGTTTAATGGCTCACAG
TTCTGCAGGCTGTACAAGAAGCATGGCTGGATCAGCTTCTGGGCAGGCCATAGGGAACTTAAAATCATGATGGAA
GGCATAGGGAGACCCCAGACTTCACATGGCAGGAACTGGGGGAAGAGAGAAATGGGAGGTGCTACATACGTTTAA
ACAACTAGATCTTGTCAGAACTCACTATATAGTACCAAGAGGGGACTGTACAAAACCATTAGAAGCCACCCCATA
ATCCACTCACCTCCCACCAGGCCCAACCTCCAACACTGGGGATTACAGTTGAACATGAGATTTGGGTGGGGACAG
AGATCCAAACCATGTTATTCCAACTCTGGCCCCTCCCAAATCTAATGTCCTTCTCATATTGCAAAATACTGTCGT
GCCTTACCAACAGTTCCCCAAAGTCTTAACTCGATCCAGCATTCATTCAAAAGTCCAAAGTCCCAAGTCTCACCT
GAGACGAAGCTAGTCCCTTCTACCTATGAACCTGTAAAATCAAAAACAAGGTAATTGCTTCAAAGATACAATGGG
GGTATAGGCATTGGGCAGATACTGCCATTCCGAAAGGGAGAAATCTGCCAAAAGAAAGAGGCTATAGGGCCCCAT
TGCAAGTCTGAAAGCCAGCCGGGCAGTCATTAAATGTTAAAGCTCTGAAATAATCTCCTTTGACTCACACCCAGG
GAACACTGATGCAATGAGTGGGCTCCCAAAACCTTGGGCAGAACCACCCCTGTGGTTTTCCAGGGTTCATCTCCC
ACAGCTGCTCTCATGGGCTAGCATTGAGTGCTTGCAGCTTTTCCAGGCTGCAGGGTGCAAGTTGTTGGTGGATCT
ACCATTCTGGGGTCTGGAGGACGGTGGCTGTCTTGTCATAGCTCTGCTAGGCAGTGCCCCAGGGGACTCTCTGTG
GGGGCTGCAACCCCACATTTCTTCTCCTTGCTTCCCTAGTAGATGTTCTCCATGAGGATTCCACCCCAGTAACAG
GCTTCTGTCTGGACATCCAGGCTTTTTCATACATCCTCTAAAATCTAGGCAGAGCTTCTTAAGCCTCAACTCTTG
CATTATGTGCGCCCGCCGGCTTCACAGCTTATGGAAGCCACCAAGGCTTATGCCTGGCACCCTGTGAAGCAGCAG
CCTGAACTGTATTCTTACTGGTGAAAGTTATCTGAGTTACCAGCTGCAAATCCATGTGGGTCTGCAGCAACCTCA
ATTCTTGCCTCCTCAGAAGAAAGAATTTGACCAAGAGGCATAAGGCAGAAAAAGAGACTGCGACAAGTTTCAGAG
CAGGAGTAAAAGTTTATTAAAAAGCTTTAGAACAGGAATGAAAGGAAAGTACATTTGGAAGAGGCCCAAGTGGGC
ACCTTGGAGGTCAAGTGCCCTGTTTGACCTTGAACCTAGGATCTTATACACTGGCCTACTTCTGACATCTTGTGC
CCCTTTCCCTTGGTCCTTCCCTAAGGGTGAGCTTGCCGCATGCATGGTGCCCTGCTTGCACTTGGAAGGTGAGCG
TGTGCAGTGTGTTTACTGGAGTTGTATACATGCTTACCTGAGGCTTTCTTCCCTTTTCCGGTGGAATGCCCCCAA
AGGTCATACTTCACCATTTTGCCTCTTAATGTGCATGTTAAGCCCACTCTCTCAGTTCCTGAGATCTTATTGGAA
GCGCCCAGTTACCAATTTCAGGTGTTTCTATCTATTGAGAAGTTGCCTCTCCCTGGTGCTGGCTGCAACCAATTA
CTATTTTAGAGAGGCAGTATGACAACTGCCTGACCATCATCTGATGGTTGCCTGACATTCCTGGTGGGTGGTGGG
GACTTCTCTCTTACCCCACTCATGCCTGATTAGCTACCTACTGTAACAGTACCTGGGCCCCTTTGAGCAGCTGGG
ATTCAGGGAGCAGAGTCCCAAGGCTGTAAGAGGGAGCAGGGGCCCTAGGCCTGGCCCAGGAAATGATTCAGTCCT
CCTAGGCCTCGGGGCCTGTGATGAGAGGGACCACCATGAAAGCCTAAAATGCCTATTAGGAACACTTTTACACTG
TTGGTGGGACTGTAAACTAGTTCAACCATTGTGGAAGTCAGTGTGGCGATTCCTCAGGGATCTAGAACTAGAAAT
ACCATTTGACCCAGCCATCCCATCACTGGGTATATACCCAAAGGACTATAAATCCTGCTGCTATAAAGACACATG
CACACTATGTTTATTGTGGCACTATTCACAATAGCAAAGACTTGGAACCAACCCAAATGTCCAACAGTGATAGAC
TGGATTAAGAAAATGTGGCACATATACACCATGGAATACTATGCAGCCATAAAAAATGATGAGTTCATGTCCTTT
GTAGGGACATGGATGAAATTGGAAATCGTCATTCTCAGTAAACTATTGCAAGGTCAAAAAACCAAACACCACATG
TTCTCACTCATAGGTGGGAATTGAACAATGAGAACACATGGACACAGGAAGGGGAACATCACACTCTGGGGACTG
TTGTGGGGTGGGGGAGGGGGGAGGGATAGCATTAGGAGATATACCTAATGCTAAATGACGAGTTAATGGGTGCAG
CACACCAGCATGGCACATGTATACATATGCAACTAACCTGCACATTGTGCACATGTACCCTAAAACTTAAAGTAT
AATAATAATAAAATAAAAATAAAATAAAATAAAATGCCTGTTAGTCCTATGAGTCTTTCTCCCCATTGTTTTGGT
TATCAGCCCTTGCCTTCCTTTTAGTTATGCAAATTTATGCAGCCTGCTTGACTTCCTCTCCTGAGAACGAGCTTT
TCTTTACTACCACATGGCCAGGCTGCAAAATTTACAAACTTTTATGCTCTGCTTCCCTTTTAAGTGTAAGTTCCA
ATTTCAGGTCATTTCTGTGCTCATGCCTATGAGCATAGGCTATTAGAAGCAGCTAGGTTACTTCTTGAACACTCT
GCTGCTTAGAAATTTCTTCTGCCAGATACCCTAAATCATCATTCTTAAGTCTAAGATTTCACAGATCCCTAGAAC
AGAGGAACAATGCAGCTAAGCTCTTTGCTAAAGCATAGCAAACCTGACCTTTACTCATTCCCAATAAATTTCTCA
TTTCCATCTGAGACCTCCTCAGCCTGGACTTCACTGTCTATATCACTATCAGTATTTTGGTTACAACCACTCAAC
AAGTTCCTAGCGAGTTCCAAACTTTCTCTCATCTTTCTGTCTTCTTCTGAGCCCTCTAAAATGTTTCAACTTCTG
CCTGTTAGCCGGTTCCAAAGTCACTTCTACATTTTCAGGTATCTTTATAGCAATGCCCTTCTTCTCAGTAACAAT
TTTCTGTATTAGTCCATTCTTGCATTGCTATAAAGAAATACTTGAGACTGGGTAATTTACAAAGAAAAGAGGTTT
AATTGACTCATGGTTCTGCAGGCTGTATAGGAAGCATGGCAGCATCAGCTTCTGGGGATGCCTCAGAGAACTTAC
AA
>SEQ ID NO: 17 nucleotides 3127-5607 of NG_031977.2
GAGTTGGAATAACATGGTTTGGATCTCTGTCCCCACCCAAATCTCATGTTCAACTGTAATCCCCAGTGTTGGAGG
TTGGGCCTGGTGGGAGGTGAGTGGATTATGGGGTGGCTTCTAATGGTTTTGTACAGTCCCCTCTTGGTACTATAT
AGTGAGTTCTGACAAGATCTAGTTGTTTAAACGTATGTAGCACCTCCCATTTCTCTCTTCCCCCAGTTCCTGCCA
TGTGAAGTCTGGGGTCTCCCTATGCCTTCCATCATGATTTTAAGTTCCCTATGGCCTGCCCAGAAGCTGATCCAG
CCATGCTTCTTGTACAGCCTGCAGAACTGTGAGCCATTAAACTTTTCTTTATAAATTACCCAGTTTCAGTTATTT
CTTTATAGCAGTGTAAGAATGGACTAACACAATTATTAACGCTAGTCCTCATGTTGTACATTAAATCTCTAGATG
TATTAGACGTAACTGCAACTTTGTACCCTACCCTACAATTTTCTTTCCCCCCAAGCCCCCCAACCAAGGGTCTAC
TCTGTTTCTATAAATTCAGTTGTTTTTTAATTCCACGTATAAGTGAAGTACAACTCAGTGTAGAAACTTGGTAAA
TGCTAGCTACTTGTTATAAGCTGTCAGTCAAAATAAAAATACAGAGATGAATCTCTAAATTAAGTGATTTATTTG
GGAAGAAAGAATTGCAATTAGGGCATACATGTAGATCAGATGGTCTTCGGTATATCCACACAACAAAGAAAAGGG
GGAGGTTTTGTTAAAAAAGAGAAATGTTACATAGTGCTCTTTGAGAAAATTCATTGGCACTATTAAGGATCTGAG
GAGCTGGTGAGTTTCAACTGGTGAGTGATGGTGGTAGATAAAATTAGAGCTGCAGCAGGTCATTTTAGCAACTAT
TAGATAAAACTGGTCTCAGGTCACAACGGGCAGTTGCAGCAGCTGGACTTGGAGAGAATTACACTGTGGGAGCAG
TGTCATTTGTCCTAAGTGCTTTTCTACCCCCTACCCCCACTATTTTAGTTGGGTATAAAAAGAATGACCCAATTT
GTATGATCAACTTTCACAAAGCATAGAACAGTAGGAAAAGGGTCTGTTTCTGCAGAAGGTGTAGACGTTGAGAGC
CATTTTGTGTATTTATTCCTCCCTTTCTTCCTCGGTGAATGATTAAAACGTTCTGTGTGATTTTTAGTGATGAAA
AAGATTAAATGCTACTCACTGTAGTAAGTGCCATCTCACACTTGCAGATCAAAAGGCACACAGTTTAAAAAACCT
TTGTTTTTTTACACATCTGAGTGGTGTAAATGCTACTCATCTGTAGTAAGTGGAATCTATACACCTGCAGACCAA
AAGACGCAAGGTTTCAAAAATCTTTGTGTTTTTTACACATCAAACAGAATGGTACGTTTTTCAAAAGTTAAAAAA
AAACAACTCATCCACATATTGCAACTAGCAAAAATGACATTCCCCAGTGTGAAAATCATGCTTGAGAGAATTCTT
ACATGTAAAGGCAAAATTGCGATGACTTTGCAGGGGACCGTGGGATTCCCGCCCGCAGTGCCGGAGCTGTCCCCT
ACCAGGGTTTGCAGTGGAGTTTTGAATGCACTTAACAGTGTCTTACGGTAAAAACAAAATTTCATCCACCAATTA
TGTGTTGAGCGCCCACTGCCTACCAAGCACAAACAAAACCATTCAAAACCACGAAATCGTCTTCACTTTCTCCAG
ATCCAGCAGCCTCCCCTATTAAGGTTCGCACACGCTATTGCGCCAACGCTCCTCCAGAGCGGGTCTTAAGATAAA
AGAACAGGACAAGTTGCCCCGCCCCATTTCGCTAGCCTCGTGAGAAAACGTCATCGCACATAGAAAACAGACAGA
CGTAACCTACGGTGTCCCGCTAGGAAAGAGAGGTGCGTCAAACAGCGACAAGTTCCGCCCACGTAAAAGATGACG
CTTGGTGTGTCAGCCGTCCCTGCTGCCCGGTTGCTTCTCTTTTGGGGGCGGGGTCTAGCAAGAGCAGGTGTGGGT
TTAGGAGGTGTGTGTTTTTGTTTTTCCCACCCTCTCTCCCCACTACTTGCTCTCACAGTACTCGCTGAGGGTGAA
CAAGAAAAGACCTGATAAAGATTAACCAGAAGAAAACAAGGAGGGAAACAACCGCAGCCTGTAGCAAGCTCTGGA
ACTCAGGAGTCGCGCGCTAGGGGCCGGGGCCGGGGCCGGGGCGTGGTCGGGGGGGGCCCGGGGGCGGGCCCGGGG
CGGGGCTGCGGTTGCGGTGCCTGCGCCCGCGGCGGCGGAGGCGCAGGCGGTGGCGAGTGGGTGAGTGAGGAGGCG
GCATCCTGGCGGGTGGCTGTTTGGGGTTCGGCTGCCGGGAAGAGGCGCGGGTAGAAGCGGGGGCTCTCCTCAGAG
CTCGACGCATTTTTACTTTCCCTCTCATTTCTCTGACCGAAGCTGGGTGTCGGGCTTTCGCCTCTAGCGACTGGT
GGAATT
>SEQ ID NO: 18 Reverse complement of SEQ ID NO: 17 (hg38_dna
range = chr9: 27573260-27575740)
AATTCCACCAGTCGCTAGAGGCGAAAGCCCGACACCCAGCTTCGGTCAGAGAAATGAGAGGGAAAGTAAAAATGC
GTCGAGCTCTGAGGAGAGCCCCCGCTTCTACCCGCGCCTCTTCCCGGCAGCCGAACCCCAAACAGCCACCCGCCA
GGATGCCGCCTCCTCACTCACCCACTCGCCACCGCCTGCGCCTCCGCCGCCGCGGGCGCAGGCACCGCAACCGCA
GCCCCGCCCCGGGCCCGCCCCCGGGCCCGCCCCGACCACGCCCCGGCCCCGGCCCCGGCCCCTAGCGCGCGACTC
CTGAGTTCCAGAGCTTGCTACAGGCTGCGGTTGTTTCCCTCCTTGTTTTCTTCTGGTTAATCTTTATCAGGTCTT
TTCTTGTTCACCCTCAGCGAGTACTGTGAGAGCAAGTAGTGGGGAGAGAGGGTGGGAAAAACAAAAACACACACC
TCCTAAACCCACACCTGCTCTTGCTAGACCCCGCCCCCAAAAGAGAAGCAACCGGGCAGCAGGGACGGCTGACAC
ACCAAGCGTCATCTTTTACGTGGGCGGAACTTGTCGCTGTTTGACGCACCTCTCTTTCCTAGCGGGACACCGTAG
GTTACGTCTGTCTGTTTTCTATGTGCGATGACGTTTTCTCACGAGGCTAGCGAAATGGGGCGGGGCAACTTGTCC
TGTTCTTTTATCTTAAGACCCGCTCTGGAGGAGCGTTGGCGCAATAGCGTGTGCGAACCTTAATAGGGGAGGCTG
CTGGATCTGGAGAAAGTGAAGACGATTTCGTGGTTTTGAATGGTTTTGTTTGTGCTTGGTAGGCAGTGGGCGCTC
AACACATAATTGGTGGATGAAATTTTGTTTTTACCGTAAGACACTGTTAAGTGCATTCAAAACTCCACTGCAAAC
CCTGGTAGGGGACAGCTCCGGCACTGCGGGCGGGAATCCCACGGTCCCCTGCAAAGTCATCGCAATTTTGCCTTT
ACATGTAAGAATTCTCTCAAGCATGATTTTCACACTGGGGAATGTCATTTTTGCTAGTTGCAATATGTGGATGAG
TTGTTTTTTTTTAACTTTTGAAAAACGTACCATTCTGTTTGATGTGTAAAAAACACAAAGATTTTTGAAACCTTG
CGTCTTTTGGTCTGCAGGTGTATAGATTCCACTTACTACAGATGAGTAGCATTTACACCACTCAGATGTGTAAAA
AAACAAAGGTTTTTTAAACTGTGTGCCTTTTGATCTGCAAGTGTGAGATGGCACTTACTACAGTGAGTAGCATTT
AATCTTTTTCATCACTAAAAATCACACAGAACGTTTTAATCATTCACCGAGGAAGAAAGGGAGGAATAAATACAC
AAAATGGCTCTCAACGTCTACACCTTCTGCAGAAACAGACCCTTTTCCTACTGTTCTATGCTTTGTGAAAGTTGA
TCATACAAATTGGGTCATTCTTTTTATACCCAACTAAAATAGTGGGGGTAGGGGGTAGAAAAGCACTTAGGACAA
ATGACACTGCTCCCACAGTGTAATTCTCTCCAAGTCCAGCTGCTGCAACTGCCCGTTGTGACCTGAGACCAGTTT
TATCTAATAGTTGCTAAAATGACCTGCTGCAGCTCTAATTTTATCTACCACCATCACTCACCAGTTGAAACTCAC
CAGCTCCTCAGATCCTTAATAGTGCCAATGAATTTTCTCAAAGAGCACTATGTAACATTTCTCTTTTTTAACAAA
ACCTCCCCCTTTTCTTTGTTGTGTGGATATACCGAAGACCATCTGATCTACATGTATGCCCTAATTGCAATTCTT
TCTTCCCAAATAAATCACTTAATTTAGAGATTCATCTCTGTATTTTTATTTTGACTGACAGCTTATAACAAGTAG
CTAGCATTTACCAAGTTTCTACACTGAGTTGTACTTCACTTATACGTGGAATTAAAAAACAACTGAATTTATAGA
AACAGAGTAGACCCTTGGTTGGGGGGCTTGGGGGGAAAGAAAATTGTAGGGTAGGGTACAAAGTTGCAGTTACGT
CTAATACATCTAGAGATTTAATGTACAACATGAGGACTAGCGTTAATAATTGTGTTAGTCCATTCTTACACTGCT
ATAAAGAAATAACTGAAACTGGGTAATTTATAAAGAAAAGTTTAATGGCTCACAGTTCTGCAGGCTGTACAAGAA
GCATGGCTGGATCAGCTTCTGGGCAGGCCATAGGGAACTTAAAATCATGATGGAAGGCATAGGGAGACCCCAGAC
TTCACATGGCAGGAACTGGGGGAAGAGAGAAATGGGAGGTGCTACATACGTTTAAACAACTAGATCTTGTCAGAA
CTCACTATATAGTACCAAGAGGGGACTGTACAAAACCATTAGAAGCCACCCCATAATCCACTCACCTCCCACCAG
GCCCAACCTCCAACACTGGGGATTACAGTTGAACATGAGATTTGGGTGGGGACAGAGATCCAAACCATGTTATTC
CAACTC
>SEQ ID NO: 19 nucleotides 5127-5607 of NG_031977.2
GGGTCTAGCAAGAGCAGGTGTGGGTTTAGGAGGTGTGTGTTTTTGTTTTTCCCACCCTCTCTCCCCACTACTTGC
TCTCACAGTACTCGCTGAGGGTGAACAAGAAAAGACCTGATAAAGATTAACCAGAAGAAAACAAGGAGGGAAACA
ACCGCAGCCTGTAGCAAGCTCTGGAACTCAGGAGTCGCGCGCTAGGGGCCGGGGCCGGGGCCGGGGCGTGGTCGG
GGCGGGCCCGGGGGCGGGCCCGGGGCGGGGCTGCGGTTGCGGTGCCTGCGCCCGCGGCGGCGGAGGCGCAGGCGG
TGGCGAGTGGGTGAGTGAGGAGGCGGCATCCTGGCGGGTGGCTGTTTGGGGTTCGGCTGCCGGGAAGAGGCGCGG
GTAGAAGCGGGGGCTCTCCTCAGAGCTCGACGCATTTTTACTTTCCCTCTCATTTCTCTGACCGAAGCTGGGTGT
CGGGCTTTCGCCTCTAGCGACTGGTGGAATT
>SEQ ID NO: 20 Reverse complement of SEQ ID NO: 19 (hg38_dna
range = chr9: 27573260-27573740)
AATTCCACCAGTCGCTAGAGGCGAAAGCCCGACACCCAGCTTCGGTCAGAGAAATGAGAGGGAAAGTAAAAATGC
GTCGAGCTCTGAGGAGAGCCCCCGCTTCTACCCGCGCCTCTTCCCGGCAGCCGAACCCCAAACAGCCACCCGCCA
GGATGCCGCCTCCTCACTCACCCACTCGCCACCGCCTGCGCCTCCGCCGCCGCGGGCGCAGGCACCGCAACCGCA
GCCCCGCCCCGGGCCCGCCCCCGGGCCCGCCCCGACCACGCCCCGGCCCCGGCCCCGGCCCCTAGCGCGCGACTC
CTGAGTTCCAGAGCTTGCTACAGGCTGCGGTTGTTTCCCTCCTTGTTTTCTTCTGGTTAATCTTTATCAGGTCTT
TTCTTGTTCACCCTCAGCGAGTACTGTGAGAGCAAGTAGTGGGGAGAGAGGGTGGGAAAAACAAAAACACACACC
TCCTAAACCCACACCTGCTCTTGCTAGACCC
SEQ ID NO. 21
CAGCTTCGGTCAGAGAAATGAGAGGGAAAGTAAAAATGCGTCGAGCTCT
SEQ ID NO. 22
CGCGACTCCTGAGTTCCAGAGCTTGCTACAGGC
SEQ ID NO. 23
AGGCTGCGGTTGTTTCCCTCCTTGTTT
SEQ ID NO. 24
GGTTGTTTCCCTCCTTGTTTTCTTCTGGTTAATCTTTATCAGGTCTTTTCTTGTTCACCCTCAGCGAGTACTGTG
AGAG
SEQ ID NO. 25
CTCAGCGAGTACTGTGAGAGCAAG
SEQ ID NO. 26
ACCTCCTAAACCCACACCTGCTCTTGCTAGACC
SEQ ID NO.27
CAGCTTCGGTCAGAGAAATGAGAGGGAAAGTAAAAATGCGTCGAGCTC
SEQ ID NO. 28
CGCGACTCCTGAGTTCCAGAGCTT
SEQ ID NO. 29
GACTCCTGAGTTCCAGAGCTTGCTACAGGC
SEQ ID NO. 30
GGCTGCGGTTGTTTCCCTCCTTGTTT
SEQ ID NO. 31
GGTTGTTTCCCTCCTTGTTTTCTTCTGGTTAATCTTTATCAGGTCTTTTCTTGTTCACCCTCAGCGAGTACTGTG
AGAG
SEQ ID NO. 32
CAGCTTCGGTCAGAGAAATGAGAGGGAAAGTAAAAATGCGTCGAGC
SEQ ID NO. 33
ACTCCTGAGTTCCAGAGCTTGCTACAG
SEQ ID NO. 34
CTGCGGTTGTTTCCCTCCTTGTTT
SEQ ID NO. 35
GGTTGTTTCCCTCCTTGTTTTCTTCTGGTTAATCTTT
SEQ ID NO. 36
TTTCTTCTGGTTAATCTTTATCAGGTCTTTTCTTGTTCACCCTCAGCGAG
SEQ ID NO. 37
GAGAGGGAAAGTAAAAATGCGTCG
SEQ ID NO. 38
GGTTGTTTCCCTCCTTGTTTTCTTCTGGTTAATCTTT
SEQ ID NO. 39
TTTCTTCTGGTTAATCTTTATCAGGTCTTTTCTTGTTCACCCTCAGCG
SEQ ID NO. 40
GTTTCCCTCCTTGTTTTCTTCTGGTTAATCTTT
SEQ ID NO. 41
TTTCTTCTGGTTAATCTTTATCAGGTCTTTTCTTGTTCACCCTCAGC
SEQ ID NO. 42
GTTTCCCTCCTTGTTTTCTTCTGGTT
SEQ ID NO. 43
CTCCTTGTTTTCTTCTGGTTAATCTTT
SEQ ID NO. 44
TTTCTTCTGGTTAATCTTTATCAGGTCTTTTCTTGTTCACCC
SEQ ID NO. 45
TCCTTGTTTTCTTCTGGTTAATCTTT
SEQ ID NO. 46
TTTCTTCTGGTTAATCTTTATCAGGTCTTTTCTT
SEQ ID NO. 47
ATCTTTATCAGGTCTTTTCTTGTTCACCC
SEQ ID NO. 51
TGCGTCAAACAGCGACAAGTTCCGC
SEQ ID NO. 52
GCCCACGTAAAAGATGACGCTTGGTGTGTC
SEQ ID NO. 53
CTTGCTCTCACAGTACTCGCTGAGGG
SEQ ID NO. 54
TCGCTGAGGGTGAACAAGAAAAGACCTGATAAAGATTAACCAGAAGAAAACAAGGAGG
SEQ ID NO. 55
TTACTTTCCCTCTCATTTCTCTGACCG
SEQ ID NO. 56
TCCCTCTCATTTCTCTGACCGAAGCT
SEQ ID NO. 57
GGAGACGCCTGCACAATTTCAGCCCAAGCTTCTAGAGAGT
SEQ ID NO. 58
CAGCCCAAGCTTCTAGAGAGTGGTGATGACTTGC
SEQ ID NO. 59
TAGAGAGTGGTGATGACTTGCATATGAGG
SEQ ID NO. 60
CTGTGGGACATGACCTGGTTGCTT
SEQ ID NO. 61
GGACATGACCTGGTTGCTTCACAGCTCC
SEQ ID NO. 62
CCTGGTTGCTTCACAGCTCCGAGATGACACAGACTTGCTTAAAGGAAGTGA
SEQ ID NO. 63
TGCGTCAAACAGCGACAAGTTCCGC
SEQ ID NO. 64
CCCACGTAAAAGATGACGCTTGGTGT
SEQ ID NO. 65
GTAAAAGATGACGCTTGGTGTGTC
SEQ ID NO. 66
CTTGCTCTCACAGTACTCGCTGAGGG
SEQ ID NO. 67
TCGCTGAGGGTGAACAAGAAAAGACCTGATAAAGATTAACCAGAAGAAAACAAGGAGG
SEQ ID NO. 68
TTACTTTCCCTCTCATTTCTCTGACCG
SEQ ID NO. 69
TCCCTCTCATTTCTCTGACCGAAGC
SEQ ID NO. 70
CGCCTGCACAATTTCAGCCCAAGCTTCTAGAGAGT
SEQ ID NO. 71
CCAAGCTTCTAGAGAGTGGTGATGA
SEQ ID NO. 72
TAGAGAGTGGTGATGACTTGCATATG
SEQ ID NO. 73
GGACATGACCTGGTTGCTTCACAGCTCC
SEQ ID NO. 74
CTCCGAGATGACACAGACTTGCTT
SEQ ID NO. 75
GAGATGACACAGACTTGCTTAAAGGAA
SEQ ID NO. 76
GCGTCAAACAGCGACAAGTTCCGC
SEQ ID NO. 77
GTAAAAGATGACGCTTGGTGTGTC
SEQ ID NO. 78
TGAACAAGAAAAGACCTGATAAAGATTAACCAGAAGAAAACAAGGAGG
SEQ ID NO. 79
TTACTTTCCCTCTCATTTCTCTGAC
SEQ ID NO. 80
CCAAGCTTCTAGAGAGTGGTGATG
SEQ ID NO. 81
ATGACCTGGTTGCTTCACAGCTCC
SEQ ID NO. 82
CTCCGAGATGACACAGACTTGCTT
SEQ ID NO. 83
GAGATGACACAGACTTGCTTAAAGGA
SEQ ID NO. 84
GCGTCAAACAGCGACAAGTTCCGC
SEQ ID NO. 85
TGAACAAGAAAAGACCTGATAAAGATTAACCAGAAGAAAACAAGGAGG
SEQ ID NO. 86
TTACTTTCCCTCTCATTTCTCTGA
SEQ ID NO. 87
ATGACCTGGTTGCTTCACAGCTCC
SEQ ID NO. 88
CTCCGAGATGACACAGACTTGCTT
SEQ ID NO. 89
GAGATGACACAGACTTGCTTAAAGGA
SEQ ID NO. 90
AGAAAAGACCTGATAAAGATTAAC
SEQ ID NO. 91
AAGACCTGATAAAGATTAACCAGA
SEQ ID NO. 92
TTACTTTCCCTCTCATTTCTCTGA
SEQ ID NO. 93
CTCCGAGATGACACAGACTTGCTT
SEQ ID NO: 94
>hg38_dna
gtttaaacTCCCCCAGGCGAGGCCTCTCAGTACCCGAGGCTCCCTTTTCTCGAGCCCGCAGCGGCAGCGCTCCCA
GCGGGTCCCCGGGAAGGAGACAGCTCGGGTACTGAGGGCGGGAAAGCAAGGAAGAGGCCAGATCCCCATCCCTTG
TCCCTGCGCCGCCGCCGCCGCCGCCGCCGCCGGGAAGCCCGGGGCCCGGATGCAGGCAATTCCACCAGTCGCTAG
AGGCGAAAGCCCGACACCCAGCTTCGGTCAGAGAAATGAGAGGGAAAGTAAAAATGCGTCGAGCTCTGAGGAGAG
CCCCCGCTTCTACCCGCGCCTCTTCCCGGCAGCCGAACCCCAAACAGCCACCCGCCAGGATGCCGCCTCCTCACT
CACCCACTCGCCACCGCCTGCGCCTCCGCCGCCGCGGGCGCAGGCACCGCAACCGCAGCCCCGCCCCGGGCCCGC
CCCCGGGCCCGCCCCGACCACGCCCCGGCCCCGGCCCCGGCCCCTAGCGCGCGACTCCTGAGTTCCAGAGCTTGC
TACAGGCTGCGGTTGTTTCCCTCCTTGTTTTCTTCTGGTTAATCTTTATCAGGTCTTTTCTTGTTCACCCTCAGC
GAGTACTGTGAGAGCAAGTAGTGGGGAGAGAGGGTGGGAAAAACAAAAACACACACCTCCTAAACCCACACCTGC
TCTTGCTAGACCCGCGGCCGC
SEQ ID NO: 100
GGGGCC

1. A double stranded ribonucleic acid (dsRNA) agent for reducing the level of a C9orf72 antisense RNA transcript,
a) wherein the dsRNA agent comprises a sense strand and an antisense strand forming a double stranded region,
wherein the sense strand comprises at least 15 contiguous nucleotides differing by no more than 3 nucleotides from the nucleotide sequence of SEQ ID NO: 17, and the antisense strand comprises a nucleotide sequence comprising at least 15 contiguous nucleotides differing by no more than 3 nucleotides from the corresponding portion of the nucleotide sequence of SEQ ID NO:18; and
wherein the sense strand, the antisense strand, or both the sense strand and the antisense strand comprises at least one modified nucleotide;
b) wherein the dsRNA agent comprises a sense strand and an antisense strand forming a double stranded region,
wherein the sense strand comprises at least 15 contiguous nucleotides differing by no more than 3 nucleotides from the nucleotide sequence of SEQ ID NO:19, and the antisense strand comprises a nucleotide sequence comprising at least 15 contiguous nucleotides differing by no more than 3 nucleotides from the corresponding portion of the nucleotide sequence of SEQ ID NO:20; and
wherein the sense strand, the antisense strand, or both the sense strand and the antisense strand comprises at least one modified nucleotide;
c) wherein the dsRNA agent comprises a sense strand and an antisense strand forming a double stranded region,
wherein the sense strand or the antisense strand is a sense strand or an antisense strand selected from the group consisting of any of the sense strands and antisense strands in any one of Tables 2, 3, 10A, 10C, 11, and 12; and
wherein the sense strand, the antisense strand, or both the sense strand and the antisense strand comprises at least one modified nucleotide;
d) wherein the dsRNA agent comprises a sense strand and an antisense strand forming a double stranded region,
wherein the sense strand comprises at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13; and
wherein the sense strand, the antisense strand, or both the sense strand and the antisense strand comprises at least one modified nucleotide;
e) wherein the dsRNA agent comprises a sense strand and an antisense strand forming a double stranded region,
wherein the antisense strand comprises at least 15 contiguous nucleotides differing by no more than 3 nucleotides from any one of the antisense nucleotide sequences in any one of Tables 5, 6, 10B, and 10D; and
wherein the sense strand, the antisense strand, or both the sense strand and the antisense strand comprises at least one modified nucleotide;
f) wherein said dsRNA comprises a sense strand and an antisense strand forming a double stranded region,
wherein the sense strand comprises at least 15 contiguous nucleotides differing by no more than three nucleotides from any one of the nucleotide sequence of nucleotides 1-23; 15-37; 33-55; 37-59; 59-81, 62-84, or 62-91 of SEQ ID NO: 1, and the antisense strand comprises at least 15 contiguous nucleotides from the corresponding nucleotide sequence of SEQ ID NO:5; and
wherein the sense strand, the antisense strand, or both the sense strand and the antisense strand comprises at least one modified nucleotide;
g) wherein said dsRNA comprises a sense strand and an antisense strand forming a double stranded region,
wherein the sense strand comprises at least 15 contiguous nucleotides differing by no more than three nucleotides from any one of the nucleotide sequence of nucleotides 5197-5219; 5213-5235; 5223-5245, 5226-5248; 5227-5249, 5228-5250, 5229-5251, 5230-5252, 5231-5253, 5233-5255; 5235-5256, 5241-5263; 5245-5267; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and the antisense strand comprises at least 15 contiguous nucleotides from the corresponding nucleotide sequence of SEQ ID NO: 16; and
wherein the sense strand, the antisense strand, or both the sense strand and the antisense strand comprises at least one modified nucleotide;
h) wherein said dsRNA comprises a sense strand and an antisense strand forming a double stranded region,
wherein the sense strand comprises at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and the antisense strand comprises at least 15 contiguous nucleotides from the corresponding nucleotide sequence of SEQ ID NO: 16; and
wherein the sense strand, the antisense strand, or both the sense strand and the antisense strand comprises at least one modified nucleotide;
i) wherein said dsRNA comprises a sense strand and an antisense strand forming a double stranded region,
wherein the sense strand comprises at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and the antisense strand comprises at least 15 contiguous nucleotides from the corresponding nucleotide sequence of SEQ ID NO:5; and
wherein the sense strand, the antisense strand, or both the sense strand and the antisense strand comprises at least one modified nucleotide;
j) wherein said dsRNA comprises a sense strand and an antisense strand forming a double stranded region,
wherein the sense strand comprises at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-MEI 47704194v.1 27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and the antisense strand comprises at least 15 contiguous nucleotides from the corresponding nucleotide sequence of SEQ ID NO: 14; and
wherein the sense strand, the antisense strand, or both the sense strand and the antisense strand comprises at least one modified nucleotide; or
k) wherein the dsRNA agent comprises a sense strand and an antisense strand forming a double stranded region,
wherein the antisense strand comprises at least 15 contiguous nucleotides differing by no more than 3 nucleotides from any one of the antisense nucleotide sequences in any one of Tables 8 and 9; and
wherein the sense strand, the antisense strand, or both the sense strand and the antisense strand comprises at least one modified nucleotide.

2-16. (canceled)

17. The dsRNA agent of claim 1, wherein the sense strand, the antisense strand, or both the sense strand and the antisense strand is conjugated to one or more lipophilic moieties.

18. The dsRNA agent of claim 17, wherein the lipophilic moiety is conjugated to one or more internal positions in the double stranded region of the dsRNA agent.

19.-23. (canceled)

24. The dsRNA agent of claim 1, wherein all of the nucleotides of the sense strand and all of the nucleotides of the antisense strand comprise a nucleotide modification are modified nucleotides.

25. The dsRNA agent of claim 24, wherein at least one of the nucleotide modifications is selected from the group consisting of a deoxy-nucleotide modification, a 3′-terminal deoxy-thymine (dT) nucleotide modification, a 2′—O-methyl nucleotide modification, a 2′-fluoro nucleotide modification, a 2′-deoxy nucleotide modification, a 2′—O-hexadecyl nucleotide modification, a 2′-phosphate nucleotide modification, a 2′-5′-linked ribonucleotide (3′-RNA) modification, a locked nucleotide modification, an unlocked nucleotide modification, a conformationally restricted nucleotide modification, a constrained ethyl nucleotide modification, an abasic nucleotide modification, an inverted abasic residue modification, a 2′-amino nucleotide modification, a 2′—O-allyl nucleotide modification, 2′-C-alkyl nucleotide modification, 2′-hydroxy nucleotide modification, a 2′-methoxyethyl nucleotide modification, a 2′—O-alkyl nucleotide modification, 2′,3′-seco nucleotide modification, a morpholino nucleotide modification, a phosphoramidate modification, a non-natural base comprising nucleotide modification, a tetrahydropyran nucleotide modification, a 1,5-anhydrohexitol nucleotide modification, a cyclohexenyl nucleotide modification, a nucleotide comprising a 5′-phosphorothioate group modification, a nucleotide comprising a 5′-methylphosphonate group modification, a nucleotide comprising a 5′ phosphate or 5′ phosphate mimic modification, a nucleotide comprising vinyl phosphonate modification, a nucleotide comprising glycol nucleic acid (GNA) modification, a nucleotide comprising a glycol nucleic acid S-Isomer (S-GNA) modification, a nucleotide comprising 2-hydroxymethyl-tetrahydrofuran-5-phosphate modification, a nucleotide comprising 2′-deoxythymidine-3′phosphate modification, a nucleotide comprising 2′-deoxyguanosine-3′-phosphate modification, and a terminal nucleotide linked to a cholesteryl derivative and a dodecanoic acid bisdecylamide group modification; and combinations thereof.

26-28. (canceled)

29. The dsRNA agent of claim 1, comprising at least one phosphorothioate internucleotide linkage.

30. (canceled)

31. The dsRNA agent of claim 1, wherein each strand is no more than 30 nucleotides in length.

32. (canceled)

33. (canceled)

34. The dsRNA agent of claim 1, wherein the double stranded region is 15-30 nucleotide pairs in length.

35-51. (canceled)

52. The dsRNA agent of claim 18, wherein the one or more lipophilic moieties are conjugated to one or more of the internal positions selected from the group consisting of positions 4-8 and 13-18 on the sense strand, and positions 6-10 and 15-18 on the antisense strand, counting from the 5′-end-5′end of each strand.

53-61. (canceled)

62. The dsRNA agent of claim 18, wherein the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne.

63-78. (canceled)

79. The dsRNA agent of claim 1, further comprising a phosphate or phosphate mimic at the 5′-end of the antisense strand.

80-82. (canceled)

83. A cell containing the dsRNA agent of claim 1.

84. A pharmaceutical composition for inhibiting expression of a C9orf72, comprising the dsRNA agent of claim 1.

85-90. (canceled)

91. A composition comprising two or more double stranded ribonucleic acid (dsRNA) agents for inhibiting expression of C9orf72,
wherein each dsRNA agent independently comprises a sense strand and an antisense strand forming a double stranded region,
wherein a first dsRNA agent targeting the antisense strand of C9orf72 is selected from the group consisting of
a) a dsRNA agent comprising a sense strand comprising at least 15 contiguous nucleotides differing by no more than 3 nucleotides from the nucleotide sequence of SEQ ID NO:17 and an antisense strand comprising a nucleotide sequence comprising at least 15 contiguous nucleotides differing by no more than 3 nucleotides from the corresponding portion of the nucleotide sequence of SEQ ID NO:18,
b) a dsRNA agent comprising a sense strand comprising at least 15 contiguous nucleotides differing by no more than 3 nucleotides from the nucleotide sequence of SEQ ID NO:19 and an antisense strand comprising a nucleotide sequence comprising at least 15 contiguous nucleotides differing by no more than 3 nucleotides from the corresponding portion of the nucleotide sequence of SEQ ID NO:20,
c) a dsRNA agent comprising an antisense comprising a nucleotide sequence selected from the group consisting of any of the antisense strand nucleotide sequences in any one of Tables 2, 3, 10A, 10C, 11, and 12;
d) a dsRNA agent comprising a sense strand comprising at least 15 contiguous nucleotides differing by no more than three nucleotides from nucleotides 27573296-27573318; 27573314-27573336; 27573319-27573341; 27573562-27573584; 27573585-27573607; 27573592-27573614; 27573599-27573621; 27573608-27573630; 27573616-27573638; 27573619-27573641; 27573622-27573644; 27573633-27573655; 27573690-27573712; or 27573717-27573739 of SEQ ID NO: 13; and
e) a dsRNA agent comprising a sense strand comprising at least 15 contiguous nucleotides differing by no more than three nucleotides from any one of the nucleotide sequence of nucleotides 27573296-27573584; 27573296-27573575; 27573301-27573338; 27573318-27573342; 27573555-27573583; 27573581-27573607; 27573584-27573607; 27573588-27573671; 27573588-27573666; 27573588-27573624; 27573592-27573624; 27573592-27573617; 27573598-27573624; 27573599-27573623; 27573606-27573655; 27573606-27573652; 27573606-27573647; 27573654-27573712; or 27573707-27573740 of SEQ ID NO: 13, and an antisense strand comprising at least 15 contiguous nucleotides from the corresponding nucleotide sequence of SEQ ID NO:14; and
wherein a second dsRNA agent targeting the sense strand of C9orf72 is selected from the group consisting of
a) a dsRNA agent comprising a sense strand comprising at least 15 contiguous nucleotides differing by no more than 3 nucleotides from the nucleotide sequence of SEQ ID NO:1 and an antisense strand comprising a nucleotide sequence comprising at least 15 contiguous nucleotides differing by no more than 3 nucleotides from the corresponding portion of the nucleotide sequence of SEQ ID NO:5,
b) a dsRNA agent comprising a sense strand comprising at least 15 contiguous nucleotides differing by no more than 3 nucleotides from the nucleotide sequence of SEQ ID NO:15 and an antisense strand comprising a nucleotide sequence comprising at least 15 contiguous nucleotides differing by no more than 3 nucleotides from the corresponding portion of the nucleotide sequence of SEQ ID NO:16,
c) a dsRNA agent comprising an antisense strand comprising at least 15 contiguous nucleotides differing by no more than 3 nucleotides from any one of the antisense nucleotide sequences in any one of Tables 5, 6, 10B, and 10D;
d) a dsRNA agent comprising a sense strand comprising at least 15 contiguous nucleotides differing by no more than three nucleotides from any one of the nucleotide sequence of nucleotides 1-23; 15-37; 33-55; 37-59; 59-81; 62-84, or 69-91 of SEQ ID NO: 1, and an antisense strand comprising at least 15 contiguous nucleotides from the corresponding nucleotide sequence of SEQ ID NO:5;
e) a dsRNA agent comprising a sense strand comprising at least 15 contiguous nucleotides differing by no more than three nucleotides from any one of the nucleotide sequence of nucleotides 5197-5219; 5213-5235; 5223-5245; 5226-5248; 5227-5249; 5228-5250, 5229-5251, 5230-5252, 5231-5253, 5233-5255; 5235-5256, 5241-5263; 5245-5267; 5233-5255; 5248-5270; 5539-5561; 5547-5569; 5917-5939; 5936-5958; 5954-5976; 6008-6030; 6021-6043; 6036-6058; 6043-6065; or 6048-6070 of SEQ ID NO: 15, and an antisense strand comprising at least 15 contiguous nucleotides from the corresponding nucleotide sequence of SEQ ID NO: 16;
f) a dsRNA agent comprising a sense strand comprising at least 15 contiguous nucleotides differing by no more than three nucleotides from any one of the nucleotide sequence of nucleotides 5015-5052; 5017-5040; 5032-5059; 5032-5055; 5033-5055; 5035-5059; 5036-5059; 5058-5087; 5059-5087; 5059-5084; 5064-5087; 5197-5222; 5213-5267; 5223-5252; 5229-5252; 5233-5263; 5516-5570; 5539-5565; 5539-5562; 5545-5570; 5545-5569; 5593-5616; 5883-5950; 5917-5950; 5919-5950; 5923-5950; 5934-5977; 5934-5957; 5938-5977; 5938-5965; 5938-5961; 5947-5977; 5947-5973; 5972-6001; 5973-5997; 6006-6029; 6011-6070; 6011-6039; 6011-6038; 6015-6038; 6019-6045; 6019-6042; 6033-6070; 6035-6065; 6035-6059; or 6040-6063 of SEQ ID NO: 15, and an antisense strand comprising at least 15 contiguous nucleotides from the corresponding nucleotide sequence of SEQ ID NO:16;
g) a dsRNA agent comprising a sense strand comprising at least 15 contiguous nucleotides differing by no more than three nucleotides from any one of the nucleotide sequence of nucleotides 15-52; 17-40; 32-59; 32-55; 35-59; 36-59; 58-87; 59-87; 59-84; or 64-87 of SEQ ID NO: 1, and an antisense strand comprising at least 15 contiguous nucleotides from the corresponding nucleotide sequence of SEQ ID NO:5; and
h) a dsRNA agent comprising an antisense strand comprising at least 15 contiguous nucleotides differing by no more than 3 nucleotides from any one of the antisense nucleotide sequences in any one of Tables 8 and 9; and
wherein the sense strand, the antisense strand, or both the sense strand and the antisense strand of the first dsRNA, the second dsRNA agent, or both the first and second dsRNA agent comprises at least one modified nucleotide.

92-97. (canceled)

98. The composition of claim 91, wherein
a) the first dsRNA agent comprises an antisense strand comprising a nucleotide sequence comprising at least 15 contiguous nucleotides differing by no more than 3 nucleotides from the antisense sequence of AD-1446213; and the second dsRNA agent comprises an antisense strand comprising a nucleotide sequence comprising at least 15 contiguous nucleotides differing by no more than 3 nucleotides from the antisense sequence of AD-1285238;
b) the first dsRNA agent comprises an antisense strand comprising a nucleotide sequence comprising at least 15 contiguous nucleotides differing by no more than 3 nucleotides from the antisense sequence of AD-1446213; and the second dsRNA agent comprises an antisense strand comprising a nucleotide sequence comprising at least 15 contiguous nucleotides differing by no more than 3 nucleotides from the antisense sequence of AD-1285234;
c) the first dsRNA agent comprises an antisense strand comprising a nucleotide sequence comprising at least 15 contiguous nucleotides differing by no more than 3 nucleotides from the antisense sequence of AD-1446246; and the second dsRNA agent comprises an antisense strand comprising a nucleotide sequence comprising at least 15 contiguous nucleotides differing by no more than 3 nucleotides from the antisense sequence of AD-1285238;
d) the first dsRNA agent comprises an antisense strand comprising a nucleotide sequence comprising at least 15 contiguous nucleotides differing by no more than 3 nucleotides from the antisense sequence of AD-1446246; and the second dsRNA agent comprises an antisense strand comprising a nucleotide sequence comprising at least 15 contiguous nucleotides differing by no more than 3 nucleotides from the antisense sequence of AD-1285234;
e) the first dsRNA agent comprises an antisense strand comprising a nucleotide sequence comprising at least 15 contiguous nucleotides differing by no more than 3 nucleotides from the antisense sequence of AD-1446268; and the second dsRNA agent comprises an antisense strand comprising a nucleotide sequence comprising at least 15 contiguous nucleotides differing by no more than 3 nucleotides from the antisense sequence of AD-1285238;
f) the first dsRNA agent comprises an antisense strand comprising a nucleotide sequence comprising at least 15 contiguous nucleotides differing by no more than 3 nucleotides from the antisense sequence of AD-1446268; and the second dsRNA agent comprises an antisense strand comprising a nucleotide sequence comprising at least 15 contiguous nucleotides differing by no more than 3 nucleotides from the antisense sequence of AD-1285234.

99. (canceled)

100. The composition of claim 91, wherein the first dsRNA, the second dsRNA agent, or both the first and second dsRNA agents is conjugated to one or more lipophilic moieties.

101. The composition of claim 91, wherein the lipophilic moiety is conjugated to one or more internal positions in the double stranded region of the first dsRNA agent, the second dsRNA agent or both the first and second dsRNA agent.

102-106. (canceled)

107. The composition of claim 91, wherein all of the nucleotides of the sense strand and all of the nucleotides of the antisense strand of the first dsRNA agent, the second dsRNA agent or both the first and second dsRNA agent are modified nucleotides.

108. The composition of claim 91, wherein at least one of the nucleotide modifications is selected from the group consisting of a deoxy-nucleotide modification, a 3′-terminal deoxy-thymine (dT) nucleotide modification, a 2′—O-methyl nucleotide modification, a 2′-fluoro nucleotide modification, a 2′-deoxy nucleotide modification, a 2′—O-hexadecyl nucleotide modification, a 2′-phosphate nucleotide modification, a 2′-5′-linked ribonucleotide (3′-RNA) modification, a locked nucleotide modification, an unlocked nucleotide modification, a conformationally restricted nucleotide modification, a constrained ethyl nucleotide modification, an abasic nucleotide modification, an inverted abasic residue modification, a 2′-amino nucleotide modification, a 2′—O-allyl nucleotide modification, 2′-C-alkyl nucleotide modification, 2′-hydroxy nucleotide modification, a 2′-methoxyethyl nucleotide modification, a 2′—O-alkyl nucleotide modification, 2′,3′-seco nucleotide modification, a morpholino nucleotide modification, a phosphoramidate modification, a non-natural base comprising nucleotide modification, a tetrahydropyran nucleotide modification, a 1,5-anhydrohexitol nucleotide modification, a cyclohexenyl nucleotide modification, a nucleotide comprising a 5′-phosphorothioate group modification, a nucleotide comprising a 5′-methylphosphonate group modification, a nucleotide comprising a 5′ phosphate or 5′ phosphate mimic modification, a nucleotide comprising vinyl phosphonate modification, a nucleotide comprising glycol nucleic acid (GNA) modification, a nucleotide comprising a glycol nucleic acid S-Isomer (S-GNA) modification, a nucleotide comprising 2-hydroxymethyl-tetrahydrofuran-5-phosphate modification, a nucleotide comprising 2′-deoxythymidine-3′phosphate modification, a nucleotide comprising 2′-deoxyguanosine-3′-phosphate modification, and a terminal nucleotide linked to a cholesteryl derivative and a dodecanoic acid bisdecylamide group modification; and combinations thereof.

109-111. (canceled)

112. The composition of claim 91, wherein the first dsRNA agent, the second dsRNA agent or both the first and second dsRNA agents comprise at least one phosphorothioate internucleotide linkage.

113. (canceled)

114. The composition of claim 91, wherein each strand of the first dsRNA agent, the second dsRNA agent or both the first and second dsRNA agents is no more than 30 nucleotides in length.

115. (canceled)

116. (canceled)

117. The composition of claim 91, wherein the double stranded region of the first dsRNA agent, the second dsRNA agent or both the first and second dsRNA agent is 15-30 nucleotide pairs in length.

118-135. (canceled)

136. The composition of claim 101, wherein the one or more lipophilic moieties are conjugated to one or more of the internal positions of the first dsRNA agent, the second dsRNA agent or both the first and second dsRNA agents selected from the group consisting of positions 4-8 and 13-18 on the sense strand, and positions 6-10 and 15-18 on the antisense strand, counting from the 5′-end of each strand.

137-145. (canceled)

146. The composition of claim 101, wherein the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne.

147-162. (canceled)

163. The composition of claim 91, wherein the first dsRNA agent, the second dsRNA agent or both the first and second dsRNA agents further comprises a phosphate or phosphate mimic at the 5′-end of the antisense strand.

164.-166. (canceled)

167. A cell containing the composition of claim 91.

168. (canceled)

169. (canceled)

170. A method of reducing the level of one or more C9orf72 RNA transcripts in a cell, the method comprising contacting the cell with any one or more of the dsRNA agents of claim 1, thereby inhibiting expression of C9orf72 in the cell.

171-180. (canceled)

181. A method of treating a subject having a disorder that would benefit from reduction in C9orf72 expression, comprising administering to the subject a therapeutically effective amount of any one or more of the dsRNA agents of claim 1, thereby treating the subject having the disorder that would benefit from reduction in C9orf72 expression.

182. (canceled)

183. The method of claim 181, wherein the disorder is a C9orf72-associated disorder.

184-197. (canceled)

198. A kit, a vial, or a syringe comprising any one or more of the dsRNA agents of claim 1.

199. (canceled)

200. (canceled)
